SEQUENCE LISTING (401 sequences)

SEQ ID NO: 1 - protein, 502 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 485 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 677 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 481 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 464 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 657 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - RNA, 10 nt, synthetic construct
  source 1..10: mol_type = other RNA
agcataaaag

SEQ ID NO: 8 - RNA, 15 nt, synthetic construct
  source 1..15: mol_type = other RNA
gaattcgctgccacc

SEQ ID NO: 9-10 - skipped (2 entries)

SEQ ID NO: 11 - RNA, 79 nt, Mus musculus
  source 1..79: mol_type = other RNA
agcataaaagtcacatttgcttctgacatagttgtgttgactcacaaccccagaaacagacatcgaattcgctgccacc

SEQ ID NO: 12 - RNA, 97 nt, unidentified
  source 1..97: mol_type = other RNA
agcataaaagctcggtcctgggagggtttaaaagccagggggccgtctcgacctcagtctgaccttccctcccggcagcagcgaattcgctgccacc

SEQ ID NO: 13 - RNA, 117 nt, unidentified
  source 1..117: mol_type = other RNA
agcataaaagggctgctccttgctatagaagacctgggacagaggactgctgtctgccctctctggtcaccctgcctagctagaggatctgtgaccccagccgaattcgctgccacc

SEQ ID NO: 14 - RNA, 66 nt, Homo sapiens
  source 1..66: mol_type = other RNA
agcataaaagctagcttttctcttctgtcaaccccacacgcctttggcacagaattcgctgccacc

SEQ ID NO: 15 - RNA, 65 nt, unidentified
  source 1..65: mol_type = other RNA
agcataaaagactagtattcttctggtccccacagactcagagagaacccgaattcgctgccacc

SEQ ID NO: 16 - RNA, 76 nt, synthetic construct
  source 1..76: mol_type = other RNA
agcataaaagactcactatttgttttcgcgcccagttgcaaaaagtgtcgtaattgactaagaattcgctgccacc

SEQ ID NO: 17 - RNA, 76 nt, synthetic construct
  source 1..76: mol_type = other RNA
agcataaaaggcttctagcaataggctataattcttcatcgtgttgtaacaagcgaactctgaattcgctgccacc

SEQ ID NO: 18 - RNA, 73 nt, synthetic construct
  source 1..73: mol_type = other RNA
agcataaaagaaataagagagaaaagaagagtaagaagaaatataagaccccggcgccgaattcgctgccacc

SEQ ID NO: 19 - RNA, 157 nt, Tobacco etch virus
  source 1..157: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccacc

SEQ ID NO: 20 - RNA, 113 nt, unidentified
  source 1..113: mol_type = other RNA
agcataaaagaactttcccccctcggcgccccaccggctcccgcgcgcctcccctcgcgcccgagcttcgagccaagcagcgtcctggggagcgcgtcgaattcgctgccacc

SEQ ID NO: 21 - RNA, 54 nt, Mus musculus
  source 1..54: mol_type = other RNA
tcacatttgcttctgacatagttgtgttgactcacaaccccagaaacagacatc

SEQ ID NO: 22 - RNA, 72 nt, unidentified
  source 1..72: mol_type = other RNA
ctcggtcctgggagggtttaaaagccagggggccgtctcgacctcagtctgaccttccctcccggcagcagc

SEQ ID NO: 23 - RNA, 92 nt, unidentified
  source 1..92: mol_type = other RNA
ggctgctccttgctatagaagacctgggacagaggactgctgtctgccctctctggtcaccctgcctagctagaggatctgtgaccccagcc

SEQ ID NO: 24 - RNA, 41 nt, Homo sapiens
  source 1..41: mol_type = other RNA
ctagcttttctcttctgtcaaccccacacgcctttggcaca

SEQ ID NO: 25 - RNA, 40 nt, unidentified
  source 1..40: mol_type = other RNA
actagtattcttctggtccccacagactcagagagaaccc

SEQ ID NO: 26 - RNA, 51 nt, synthetic construct
  source 1..51: mol_type = other RNA
actcactatttgttttcgcgcccagttgcaaaaagtgtcgtaattgactaa

SEQ ID NO: 27 - RNA, 51 nt, synthetic construct
  source 1..51: mol_type = other RNA
gcttctagcaataggctataattcttcatcgtgttgtaacaagcgaactct

SEQ ID NO: 28 - RNA, 48 nt, synthetic construct
  source 1..48: mol_type = other RNA
aaataagagagaaaagaagagtaagaagaaatataagaccccggcgcc

SEQ ID NO: 29 - RNA, 132 nt, Tobacco etch virus
  source 1..132: mol_type = other RNA
tctcaacacaacatatacaaaacaaacgaatctcaagcaatcaagcattctacttctattgcagcaatttaaatcatttcttttaaagcaaaagcaattttctgaaaattttcaccatttacgaacgatagc

SEQ ID NO: 30 - RNA, 88 nt, unidentified
  source 1..88: mol_type = other RNA
aactttcccccctcggcgccccaccggctcccgcgcgcctcccctcgcgcccgagcttcgagccaagcagcgtcctggggagcgcgtc

SEQ ID NO: 31 - RNA, 69 nt, unidentified
  source 1..69: mol_type = other RNA
aggatccgagactactgttatttgaccaatgaattgacttctaataaaatctagtggtgatgctgtaca

SEQ ID NO: 32 - RNA, 70 nt, unidentified
  source 1..70: mol_type = other RNA
aggatccagttcagacttcaaatagtggcaaataaaaagtgctatttgtgatggtttgcttctgtgtaca

SEQ ID NO: 33 - RNA, 126 nt, unidentified
  source 1..126: mol_type = other RNA
aggatccagttcagacttcaaatagtggcaaataaaaagtgctatttgtgatggtttgcttctggagactactgttatttgaccaatgaattgacttctaataaaatctagtggtgatgctgtaca

SEQ ID NO: 34 - RNA, 126 nt, unidentified
  source 1..126: mol_type = other RNA
aggatccgagactactgttatttgaccaatgaattgacttctaataaaatctagtggtgatgcagttcagacttcaaatagtggcaaataaaaagtgctatttgtgatggtttgcttctgtgtaca

SEQ ID NO: 35 - RNA, 124 nt, Homo sapiens
  source 1..124: mol_type = other RNA
aggatccgctggagcctcggtggccatgcttcttgccccttgggcctccccccagcccctcctccccttcctgcacccgtacccccgtggtctttgaataaagtctgagtgggcggcatgtaca

SEQ ID NO: 36 - RNA, 190 nt, unidentified
  source 1..190: mol_type = other RNA
aggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtaca

SEQ ID NO: 37 - RNA, 109 nt, unidentified
  source 1..109: mol_type = other RNA
aggatccgctgaccctttgacacccttcttgtcctcctctctggccccatccaggcaaccaaataaagtttgagtgtaccaggaacagaatcctggggcttgctgtaca

SEQ ID NO: 38 - RNA, 134 nt, unidentified
  source 1..134: mol_type = other RNA
aggatccgcttgcagaaaaagaaaaatgagctcaaaatttgctttgagagctacagggaattgctattactcctgtaccttctgctcaatttcctttcctcatctcaaataaatgccttgttacaagatgtaca

SEQ ID NO: 39 - RNA, 296 nt, unidentified
  source 1..296: mol_type = other RNA
aggatccctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggatgtaca

SEQ ID NO: 40 - RNA, 171 nt, Xenopus sp.
  source 1..171: mol_type = other RNA
aggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtaca

SEQ ID NO: 41 - RNA, 56 nt, unidentified
  source 1..56: mol_type = other RNA
gagactactgttatttgaccaatgaattgacttctaataaaatctagtggtgatgc

SEQ ID NO: 42 - RNA, 57 nt, unidentified
  source 1..57: mol_type = other RNA
agttcagacttcaaatagtggcaaataaaaagtgctatttgtgatggtttgcttctg

SEQ ID NO: 43 - RNA, 113 nt, unidentified
  source 1..113: mol_type = other RNA
agttcagacttcaaatagtggcaaataaaaagtgctatttgtgatggtttgcttctggagactactgttatttgaccaatgaattgacttctaataaaatctagtggtgatgc

SEQ ID NO: 44 - RNA, 113 nt, unidentified
  source 1..113: mol_type = other RNA
gagactactgttatttgaccaatgaattgacttctaataaaatctagtggtgatgcagttcagacttcaaatagtggcaaataaaaagtgctatttgtgatggtttgcttctg

SEQ ID NO: 45 - RNA, 111 nt, Homo sapiens
  source 1..111: mol_type = other RNA
gctggagcctcggtggccatgcttcttgccccttgggcctccccccagcccctcctccccttcctgcacccgtacccccgtggtctttgaataaagtctgagtgggcggca

SEQ ID NO: 46 - RNA, 177 nt, unidentified
  source 1..177: mol_type = other RNA
caaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggca

SEQ ID NO: 47 - RNA, 96 nt, unidentified
  source 1..96: mol_type = other RNA
gctgaccctttgacacccttcttgtcctcctctctggccccatccaggcaaccaaataaagtttgagtgtaccaggaacagaatcctggggcttgc

SEQ ID NO: 48 - RNA, 121 nt, unidentified
  source 1..121: mol_type = other RNA
gcttgcagaaaaagaaaaatgagctcaaaatttgctttgagagctacagggaattgctattactcctgtaccttctgctcaatttcctttcctcatctcaaataaatgccttgttacaaga

SEQ ID NO: 49 - RNA, 283 nt, unidentified
  source 1..283: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctgga

SEQ ID NO: 50 - RNA, 140 nt, Xenopus sp.
  source 1..140: mol_type = other RNA
ggttaccactaaaccagcctcaagaacacccgaatggagtctctaagctacataataccaacttacacttacaaaatgttgtcccccaaaatgtagccattcgtatctgctcctaataaaaagaaagtttcttcacattc

SEQ ID NO: 51 - RNA, 164 nt, Xenopus sp.
  source 1..164: mol_type = other RNA
ggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattc

SEQ ID NO: 52 - RNA, 1512 nt, synthetic construct
  source 1..1512: mol_type = other RNA
atggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcttcgtgcctgtgttcctgcctgccaagcctaccacaacacccgctcctagacctccaacaccagctccaacaatcgccagccagcctctgtctctgaggccagaagcttgtagacctgctgctggcggagccgtgcatacaagaggactggatttcgcctgcgacatctacatctgggctcctctggccggaacatgcggagtgctgctgctgagcctggtcatcaccctgtactgcaaccaccggaacagaagcaagcggagcaggctgctgcacagcgactacatgaacatgacccctagacggcccggacctaccagaaagcactaccagccttacgctcctcctcgggactttgccgcctatcggagcagagtgaagttcagcagatccgccgatgctcccgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataa

SEQ ID NO: 53 - RNA, 1512 nt, synthetic construct
  source 1..1512: mol_type = other RNA
atggccctgcccgttaccgccctgctcctgccgctggctctactgctgcacgccgctagacccgagatcgtgttaacgcagagccccgggacccttagcttgagcccgggagagagagccaccctgagctgcagagcttctcagagcgtgagcagcagctacctggcctggtatcagcagaagcccggccaagcccctagactgctgatctacggcgctagcagcagagccaccggcatccccgacagattctccggcagcggcagcggcaccgatttcaccctgaccatcagcagactggagcccgaggacttcgcggtgtactactgccaacaatacggcagcagcagattcaccttcggccccggcaccaaggtggacatcaaagggtccacctcgggcagcggcaagcctggaagcggagaaggctccaccaagggtcaagtgcagctggtgcagagcggcgccgaggtgaagaaacccggcagcagcgttaaggtgagctgcaaggacagcggcggcaccttcagcagctacgccatcagctgggtgagacaagcccccggccaaggcctggagtggatgggcggcatcatccccatcttcggcaccaccaactacgctcagcagttccaaggcagagtgaccatcaccgccgacgagagcacaagcaccgcctacatggagctgagcagcctgagatccgaggacaccgctgtctactattgcgcccgggaagccgtagccgccgattggctcgacccttggggccaaggcaccctggtgaccgtgagcagcttcgtgcccgtgtttctgcccgccaagccgaccaccacccccgcccctagacccccaacccccgcaccgacaatagcaagccaaccgctttcgctcagacctgaggcatgtaggcccgcggctgggggcgccgtgcacacaagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccctagaagacccggccccacaagaaagcactatcagccctacgccccccctagagacttcgccgcctacagaagcagagtgaagttcagcagaagcgccgacgcccccgcctatcagcaagggcagaatcagctttataacgagctgaaccttggcagaagagaggagtacgacgtgctggacaagagaagaggccgggaccccgagatgggcggcaagcctagaagaaagaacccccaagagggcctttacaacgagcttcagaaagacaagatggccgaggcctacagcgagatcggcatgaagggcgagagaagaagaggcaagggccacgacggcctgtaccaaggcctgagcaccgccaccaaggacacctacgacgccctgcacatgcaagccctgccccctagatgataa

SEQ ID NO: 54 - RNA, 1509 nt, synthetic construct
  source 1..1509: mol_type = other RNA
atggcgctgccagtaacggcccttctgcttcctctcgccctcctcttgcatgcggcacgcccggaaattgttcttacacaaagtcctggtacgctgtctcttagccccggagaaagagcgacgttgagttgtagagcaagccaaagtgtatctagctcctacctcgcctggtaccaacagaagcccggacaggcgccccgcctgctcatctacggtgcgagtagtagggccacgggcattcctgatcgattctccggtagtggatcaggtactgattttactcttactatctcacggcttgagccggaagattttgcagtgtattactgccagcaatatgggagcagcaggttcacgttcggacctgggacaaaagtcgacatcaaaggcagcacatccggtagcggaaagcccggttcaggggagggctctacgaagggccaggtccaactcgtgcaatccggggcggaggtcaagaagccaggctctagtgtaaaagtcagttgtaaagacagcggcggcacttttagcagttatgctatcagttgggtccgacaggctccgggccagggtttggagtggatgggaggtatcataccgatattcgggactacaaactacgcgcaacaatttcagggtcgcgtgacgatcaccgccgatgagtccacgagcacggcttatatggaactgagctcactccggtctgaagacaccgcggtttattattgcgcacgcgaagcagtcgcagctgactggctggacccctggggacagggtaccctggttacggtctctagcttcgtccccgtgtttctgcctgctaaaccgacgacaactcctgcacctcgcccacccacaccagcaccgacgattgcatcccaacctctgtcacttcgaccagaggcttgcaggccggccgctggtggtgcggttcacacgcggggattggacttcgcttgcgatatttacatctgggcgccgctcgcggggacgtgtggagtacttctcctgtcccttgtaatcactttgtactgcaaccatcgcaatcgcagcaaacgcagtagacttcttcactctgattacatgaacatgacgccgcgacgaccgggtccaacgcggaagcactatcaaccttatgcacccccccgagacttcgctgcttaccgaagtcgggtaaaatttagtagatctgctgacgctcctgcgtatcaacagggacagaaccaactgtataacgagctgaatcttggccgacgcgaggagtatgacgtactcgataagcgccggggtagagaccccgagatgggcggcaagcccagacggaagaacccacaagagggattgtataacgagttgcaaaaagacaaaatggccgaggcctactccgaaataggtatgaagggcgaacgccgccgaggtaaaggtcacgacggactttaccagggactttctacggcaacgaaggacacatacgacgcgcttcatatgcaggcacttccaccaaggtga

SEQ ID NO: 55 - RNA, 1512 nt, synthetic construct
  source 1..1512: mol_type = other RNA
atggcgctgccagtaacggcccttctgcttcctctcgccctcctcttgcatgcggcacgcccggaaattgttcttacacaaagtcctggtacgctgtctcttagccccggagaaagagcgacgttgagttgtagagcaagccaaagtgtatctagctcctacctcgcctggtaccaacagaagcccggacaggcgccccgcctgctcatctacggtgcgagtagtagggccacgggcattcctgatcgattctccggtagtggatcaggtactgattttactcttactatctcacggcttgagccggaagattttgcagtgtattactgccagcaatatgggagcagcaggttcacgttcggacctgggacaaaagtcgacatcaaaggcagcacatccggtagcggaaagcccggttcaggggagggctctacgaagggccaggtccaactcgtgcaatccggggcggaggtcaagaagccaggctctagtgtaaaagtcagttgtaaagacagcggcggcacttttagcagttatgctatcagttgggtccgacaggctccgggccagggtttggagtggatgggaggtatcataccgatattcgggactacaaactacgcgcaacaatttcagggtcgcgtgacgatcaccgccgatgagtccacgagcacggcttatatggaactgagctcactccggtctgaagacaccgcggtttattattgcgcacgcgaagcagtcgcagctgactggctggacccctggggacagggtaccctggttacggtctctagcttcgtccccgtgtttctgcctgctaaaccgacgacaactcctgcacctcgcccacccacaccagcaccgacgattgcatcccaacctctgtcacttcgaccagaggcttgcaggccggccgctggtggtgcggttcacacgcggggattggacttcgcttgcgatatttacatctgggcgccgctcgcggggacgtgtggagtacttctcctgtcccttgtaatcactttgtactgcaaccatcgcaatcgcagcaaacgcagtagacttcttcactctgattacatgaacatgacgccgcgacgaccgggtccaacgcggaagcactatcaaccttatgcacccccccgagacttcgctgcttaccgaagtcgggtaaaatttagtagatctgctgacgctcctgcgtatcaacagggacagaaccaactgtataacgagctgaatcttggccgacgcgaggagtatgacgtactcgataagcgccggggtagagaccccgagatgggcggcaagcccagacggaagaacccacaagagggattgtataacgagttgcaaaaagacaaaatggccgaggcctactccgaaataggtatgaagggcgaacgccgccgaggtaaaggtcacgacggactttaccagggactttctacggcaacgaaggacacatacgacgcgcttcatatgcaggcacttccaccaaggtgataa

SEQ ID NO: 56 - RNA, 1512 nt, synthetic construct
  source 1..1512: mol_type = other RNA
atggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataa

SEQ ID NO: 57 - RNA, 1512 nt, synthetic construct
  source 1..1512: mol_type = other RNA
atggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtgcagctggtgcagtctggggctgaggtgaagaagcctgggtcctcggtgaaggtctcctgcaaggattctggaggcaccttcagcagctatgctatcagctgggtgcgacaggcccctggacaagggcttgagtggatgggagggatcatccctatctttggtacaacaaactacgcacagcagttccagggcagagtcacgattaccgcggacgaatccacgagcacagcctacatggagctgagcagcctgagatctgaggacacggccgtgtattactgtgcgagagaggccgtggccgccgactggctggacccctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 58 - RNA, 1512 nt, synthetic construct
  source 1..1512: mol_type = other RNA
atggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 59 - RNA, 1512 nt, synthetic construct
  source 1..1512: mol_type = other RNA
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgataa

SEQ ID NO: 60 - RNA, 1461 nt, synthetic construct
  source 1..1461: mol_type = other RNA
atggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataa

SEQ ID NO: 61 - RNA, 1461 nt, synthetic construct
  source 1..1461: mol_type = other RNA
atggccctgcccgttaccgcactgcttctgccactggccctactactgcatgccgctagacccgagatagtgttaacgcagagccccggcacactgagcctgtcacccggcgagagagccaccctgagctgcagagcttctcagagcgtgagcagcagctacctggcctggtatcagcagaagcccggccaagcccctagactgctgatctacggcgctagcagcagagccaccggcatccccgacagattcagtgggagtggtagcggcaccgatttcaccctgaccatcagcagactggagcccgaggacttcgcggtgtactactgtcagcaatacggcagcagcagattcaccttcggccccggcaccaaggtcgacatcaaaggaagcacgtcgggttccggtaagcccggcagtggcgaaggatcgacaaagggccaagtgcagctggtgcagagcggcgccgaggtgaagaagcccggcagcagcgtgaaggtgagctgcaaggacagcggcggcaccttcagcagctacgccatcagctgggtgagacaagcccccggccaaggcctggagtggatgggcggcatcatccccatcttcggcaccaccaactacgctcagcagttccaaggcagagtgaccatcaccgccgacgagagcacaagcaccgcctacatggagctgagcagcctgagaagcgaggacaccgccgtgtactactgtgcccgggaagccgtggccgccgactggttagatccctggggccaaggcaccctggtgaccgtgagcagcatcgaggtgatgtacccccccccctacctggacaacgaaaagagcaacggcaccatcatccacgtgaagggcaagcacctgtgccctagccccctgttccccggcccaagcaaaccattctgggtgctggtggtggtgggcggcgtgctggcctgctacagcctgctggttacggtagctttcatcatcttctgggtgagaagcaagagaagcagactgctgcacagcgactacatgaacatgacccctagaagacccggccccacaagaaagcactatcagccctacgccccccctagagacttcgccgcctacagaagcagagtgaagttcagcagaagcgccgacgcccccgcctatcagcaagggcagaatcagctctacaacgagctgaacctgggcagaagagaggagtacgacgtgctggacaagagaagaggccgggaccccgagatgggcggcaagcctagaagaaagaacccccaagagggcctctacaacgaactgcaaaaggacaagatggccgaggcctacagcgagatcggcatgaagggcgagagaagaagaggcaagggccacgacggcctgtaccaaggcctgagcaccgccaccaaggacacctacgacgccctgcacatgcaagccctgccccctagatgataa

SEQ ID NO: 62 - RNA, 1458 nt, synthetic construct
  source 1..1458: mol_type = other RNA
atggccctgcctgtgacggctcttctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtga

SEQ ID NO: 63 - RNA, 1461 nt, synthetic construct
  source 1..1461: mol_type = other RNA
atggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataa

SEQ ID NO: 64 - RNA, 1464 nt, synthetic construct
  source 1..1464: mol_type = other RNA
atggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgatgataa

SEQ ID NO: 65 - RNA, 1461 nt, synthetic construct
  source 1..1461: mol_type = other RNA
atggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataa

SEQ ID NO: 66 - RNA, 1461 nt, synthetic construct
  source 1..1461: mol_type = other RNA
atggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtgcagctggtgcagtctggggctgaggtgaagaagcctgggtcctcggtgaaggtctcctgcaaggattctggaggcaccttcagcagctatgctatcagctgggtgcgacaggcccctggacaagggcttgagtggatgggagggatcatccctatctttggtacaacaaactacgcacagcagttccagggcagagtcacgattaccgcggacgaatccacgagcacagcctacatggagctgagcagcctgagatctgaggacacggccgtgtattactgtgcgagagaggccgtggccgccgactggctggacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 67 - RNA, 1461 nt, synthetic construct
  source 1..1461: mol_type = other RNA
atggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 68 - RNA, 1461 nt, synthetic construct
  source 1..1461: mol_type = other RNA
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcatcgaagtgatgtacccccctccctacctggacaacgagaagtccaacggcaccatcatccacgtgaagggcaagcacctgtgccccagccctctgtttcctggccctagcaagcccttctgggtgctggtggtcgtgggcggagtgctggcctgttacagcctgctcgtgacagtggccttcatcatcttttgggtgcgcagcaagcggagccggctgctgcacagcgactacatgaacatgacccccagacggccaggccccaccagaaagcactaccagccttacgcccctcccagagacttcgccgcctaccgcagcagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggacacgacggcctgtaccagggactgagcaccgccaccaaggacacctacgacgccctgcacatgcaggccctgccccccagatgataa

SEQ ID NO: 69 - RNA, 2037 nt, synthetic construct
  source 1..2037: mol_type = other RNA
atggagacagacactttgctcttgtgggtactgctgctctgggtccctggaagtaccggggatattgtcttgacacaaagcccggcgatattgagtgcctccccaggtgaaaaagttactatgacatgccgagcatcttccagtgttaattatatggactggtaccagaagaaacccgggtcttcaccaaaaccttggatctacgccactagtaatctggcaagtggagtgcctgcccgcttcagcggcagtggcagcggtacttcctactcacttactatcagtcgagtagaggcagaggacgctgcgacctactactgtcagcagtggtcctttaatcctcccacattcggggggggtacaaagcttgaaataaagggttccactagtgggggtggtagtggaggcggaagcggtggaggaggaagctctgaggtgcagctccaacaatctggcgcggaactggtaaagcccggagcctccgttaaaatgagctgcaaagcaagcggctacacatttacctcttataacatgcattgggttaaacagacccctggacaaggacttgagtggatcggtgcgatatatccggggaacggggacactagctataaccagaaatttaagggcaaagccacattgacagcggacaagagcagttctacagcgtatatgcaactctcatccttgacctcagaagacagcgccgactattattgcgcaaggagcaattattacggtagctcatactggttctttgacgtgtggggggctggaactacggtaacagttagtagcgagagcaaatacggaccaccatgccccccctgccctgccccggagttcgacggcggaccgagcgtcttccttttcccccccaagcctaaagacactttgatgatcagtcggacaccggaagttacatgcgtagtagtggatgtctctcaggaagaccccgaggtgcaatttaattggtacgtcgatggtgtcgaagtacataatgcgaagacgaaaccccgggaggaacagttccagagcacgtatcgggtagtgagtgtcttgactgtgctgcatcaagattggctgaatggtaaggagtataaatgtaaagtcagcaacaaaggtctcccgtcatctatagaaaaaacgatatccaaggctaagggccaaccgagagagccacaggtgtataccctgcccccttcccaggaggaaatgacgaaaaatcaggtctctctcacgtgtttggtgaaagggttttatccatctgatattgccgtggagtgggaatctaatggacagcctgagaacaattacaagacaacgccaccggttctcgattccgatggtagtttctttctctattcacgcttgacagtcgataagtcccgctggcaagaaggcaacgttttctcctgttctgtcatgcacgaggccctgcacaatcattacacgcagaagtccttgagtttgagtttgggtaagatgttctgggtccttgttgttgtaggcggggttctcgcctgttattctctgctcgtgaccgtggcgtttataatcttttgggttaaacgaggccgcaaaaagttgctctacatcttcaaacaaccgtttatgaggccggtgcagactacacaggaagaggatggctgctcttgcagattcccggaagaggaggaaggtgggtgcgagttgagggtgaaattttccaggtccgccgatgctccagcttaccagcaagggcagaaccaattgtataacgagctgaatcttgggcggagggaggagtatgacgtgcttgacaaacgccggggtcgcgacccagaaatgggaggtaagccacgacggaaaaacccacaagagggtctttataacgaacttcaaaaggacaaaatggcggaggcctatagtgaaataggtatgaagggggagcggagacggggtaagggtcatgatggactctatcaaggtcttagcacagcgactaaggatacctacgatgcgctccatatgcaagctctgcctccgaggtgataa

SEQ ID NO: 70 - RNA, 2037 nt, synthetic construct
  source 1..2037: mol_type = other RNA
atggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcgagagcaagtacggcccaccctgcccgccctgccccgccccagagttcgacggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttccagagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgaagcggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacggtgataa

SEQ ID NO: 71 - DNA, 1512 bp, synthetic construct
  source 1..1512: mol_type = other DNA
atggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcttcgtgcctgtgttcctgcctgccaagcctaccacaacacccgctcctagacctccaacaccagctccaacaatcgccagccagcctctgtctctgaggccagaagcttgtagacctgctgctggcggagccgtgcatacaagaggactggatttcgcctgcgacatctacatctgggctcctctggccggaacatgcggagtgctgctgctgagcctggtcatcaccctgtactgcaaccaccggaacagaagcaagcggagcaggctgctgcacagcgactacatgaacatgacccctagacggcccggacctaccagaaagcactaccagccttacgctcctcctcgggactttgccgcctatcggagcagagtgaagttcagcagatccgccgatgctcccgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataa

SEQ ID NO: 72 - DNA, 1512 bp, synthetic construct
  source 1..1512: mol_type = other DNA
atggccctgcccgttaccgccctgctcctgccgctggctctactgctgcacgccgctagacccgagatcgtgttaacgcagagccccgggacccttagcttgagcccgggagagagagccaccctgagctgcagagcttctcagagcgtgagcagcagctacctggcctggtatcagcagaagcccggccaagcccctagactgctgatctacggcgctagcagcagagccaccggcatccccgacagattctccggcagcggcagcggcaccgatttcaccctgaccatcagcagactggagcccgaggacttcgcggtgtactactgccaacaatacggcagcagcagattcaccttcggccccggcaccaaggtggacatcaaagggtccacctcgggcagcggcaagcctggaagcggagaaggctccaccaagggtcaagtgcagctggtgcagagcggcgccgaggtgaagaaacccggcagcagcgttaaggtgagctgcaaggacagcggcggcaccttcagcagctacgccatcagctgggtgagacaagcccccggccaaggcctggagtggatgggcggcatcatccccatcttcggcaccaccaactacgctcagcagttccaaggcagagtgaccatcaccgccgacgagagcacaagcaccgcctacatggagctgagcagcctgagatccgaggacaccgctgtctactattgcgcccgggaagccgtagccgccgattggctcgacccttggggccaaggcaccctggtgaccgtgagcagcttcgtgcccgtgtttctgcccgccaagccgaccaccacccccgcccctagacccccaacccccgcaccgacaatagcaagccaaccgctttcgctcagacctgaggcatgtaggcccgcggctgggggcgccgtgcacacaagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccctagaagacccggccccacaagaaagcactatcagccctacgccccccctagagacttcgccgcctacagaagcagagtgaagttcagcagaagcgccgacgcccccgcctatcagcaagggcagaatcagctttataacgagctgaaccttggcagaagagaggagtacgacgtgctggacaagagaagaggccgggaccccgagatgggcggcaagcctagaagaaagaacccccaagagggcctttacaacgagcttcagaaagacaagatggccgaggcctacagcgagatcggcatgaagggcgagagaagaagaggcaagggccacgacggcctgtaccaaggcctgagcaccgccaccaaggacacctacgacgccctgcacatgcaagccctgccccctagatgataa

SEQ ID NO: 73 - DNA, 1509 bp, synthetic construct
  source 1..1509: mol_type = other DNA
atggcgctgccagtaacggcccttctgcttcctctcgccctcctcttgcatgcggcacgcccggaaattgttcttacacaaagtcctggtacgctgtctcttagccccggagaaagagcgacgttgagttgtagagcaagccaaagtgtatctagctcctacctcgcctggtaccaacagaagcccggacaggcgccccgcctgctcatctacggtgcgagtagtagggccacgggcattcctgatcgattctccggtagtggatcaggtactgattttactcttactatctcacggcttgagccggaagattttgcagtgtattactgccagcaatatgggagcagcaggttcacgttcggacctgggacaaaagtcgacatcaaaggcagcacatccggtagcggaaagcccggttcaggggagggctctacgaagggccaggtccaactcgtgcaatccggggcggaggtcaagaagccaggctctagtgtaaaagtcagttgtaaagacagcggcggcacttttagcagttatgctatcagttgggtccgacaggctccgggccagggtttggagtggatgggaggtatcataccgatattcgggactacaaactacgcgcaacaatttcagggtcgcgtgacgatcaccgccgatgagtccacgagcacggcttatatggaactgagctcactccggtctgaagacaccgcggtttattattgcgcacgcgaagcagtcgcagctgactggctggacccctggggacagggtaccctggttacggtctctagcttcgtccccgtgtttctgcctgctaaaccgacgacaactcctgcacctcgcccacccacaccagcaccgacgattgcatcccaacctctgtcacttcgaccagaggcttgcaggccggccgctggtggtgcggttcacacgcggggattggacttcgcttgcgatatttacatctgggcgccgctcgcggggacgtgtggagtacttctcctgtcccttgtaatcactttgtactgcaaccatcgcaatcgcagcaaacgcagtagacttcttcactctgattacatgaacatgacgccgcgacgaccgggtccaacgcggaagcactatcaaccttatgcacccccccgagacttcgctgcttaccgaagtcgggtaaaatttagtagatctgctgacgctcctgcgtatcaacagggacagaaccaactgtataacgagctgaatcttggccgacgcgaggagtatgacgtactcgataagcgccggggtagagaccccgagatgggcggcaagcccagacggaagaacccacaagagggattgtataacgagttgcaaaaagacaaaatggccgaggcctactccgaaataggtatgaagggcgaacgccgccgaggtaaaggtcacgacggactttaccagggactttctacggcaacgaaggacacatacgacgcgcttcatatgcaggcacttccaccaaggtga

SEQ ID NO: 74 - protein, 489 aa, synthetic construct
MALPVTALLLPLALLLHAARPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 720 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 468 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 700 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - RNA, 1467 nt, synthetic construct
  source 1..1467: mol_type = other RNA
atggccttaccagtgaccgccttgctcctgccgctggccttgctgctccacgccgccaggccggatattgtgatgacccagactccacactcctcacctgtcacccttggacagccggcctccatatcctgcaggtctagtcaaagcctcgtatccagagatggaaacacctacttgagttggcttcagcagaggccaggccagcctccaagactcctaatttataagatttctaaccggttctctggggtcccaaacagattcagtggcagtggggcagggacagatttcacactgaaaatcagcagggtgaaagctgaggatgtcggggtttattactgcatgcaagctacacagtttcccctcaccttcggccaagggacacgactggagattaaaggcggcggaggatctggcggaggtggtagtggcggaggcggatctgaggtgcagttggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagggcgatttttggagtggttatgggggtatggacgtctggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 79 - RNA, 1467 nt, synthetic construct
  source 1..1467: mol_type = other RNA
atggccctgcccgtgaccgccctcctgctgcccctggccctgctcctgcacgccgcccggcccgacatcgtgatgacccagaccccacacagcagccccgtgaccctgggccagcccgccagcatcagctgccggagcagccagagcctggtgagccgggacggcaacacctacctgagctggctgcagcagcggcccggccagccaccccggctgctgatctacaagatcagcaaccggttcagcggcgtgcccaacaggttcagcggcagcggcgccggcaccgacttcaccctgaagatcagccgggtgaaggccgaggacgtgggcgtgtactactgcatgcaggccacccagttcccactgaccttcggccagggcacccggctggagatcaagggcggaggcggcagcggaggcggcgggagcggcggcggaggcagcgaggtgcagctggtgcagagcggcgccgaggtgaagaagcccggcgagagcctgaaaatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgacttctggagcggctacggcggcatggacgtctggggccagggcacaaccgtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccacgggacttcgccgcctaccggagcagggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccacgg

SEQ ID NO: 80 - RNA, 1467 nt, synthetic construct
  source 1..1467: mol_type = other RNA
atggctctgccggtcactgcattgttgctgccactcgctctgcttctgcacgcagcccgcccagacatagttatgacgcaaacaccccatagttctccagtgacgttgggccaacccgccagtatttcttgtagatcttctcaatctctggtatcacgagatggaaacacttacttgtcatggctccagcaaagacccggtcagcctcctcgactgctcatatataaaatatcaaatcgattcagcggtgtgccgaaccggttttcaggaagtggcgccgggacagatttcactcttaagatttccagggtgaaggcagaggacgttggagtttattattgtatgcaagctacacagtttccacttacttttggacaaggtacgcggctggaaatcaagggaggaggcggttcaggtggaggtggcagcggaggtggtggcagcgaggttcagcttgttcaatcaggggcagaggttaagaaacctggggaatctttgaaaatctcttgcaaagggtctggttatagctttacaagctattggattgggtgggtaagacagatgccggggaaaggactggaatggatgggtataatataccccggagattccgatactaggtatagtcctagcttccagggccaagtgacgatttcagcagacaaatccatctctacagcatatctgcagtggagttctcttaaggcctcagacaccgccatgtattactgcgcaagacaaggtgatttctggtccggttacggtggaatggatgtttggggccaagggactaccgtgacggtctcaagtattgaggttatgtatccacctccgtacctcgacaatgagaaatctaacggcacaatcatccatgtaaaagggaagcacctgtgtccctctcctttgttcccaggtccttctaagcctttctgggtgctcgtggtggttggaggtgtgctggcctgctactcacttttggttaccgtcgcatttattatattctgggtacggtctaaaagatctcggctgttgcacagtgattatatgaatatgaccccgagacggccgggcccaactaggaaacactaccaaccgtatgcccctcccagggactttgcggcctatcggtcccgcgttaagtttagccgcagtgcggacgcaccggcataccaacaaggtcaaaaccaactctacaatgaattgaacctcggcaggcgagaggaatacgatgttctcgataaaaggcggggtagagatcctgaaatggggggcaaacccagaaggaaaaatccccaagaaggtctctataatgagttgcaaaaagataagatggctgaggcgtactccgaaatcggaatgaagggtgagcggaggcgcggcaaaggccacgatggtctttatcagggcttgagtacagcaacgaaggatacgtacgatgctctccatatgcaggcacttcctcccagg

SEQ ID NO: 81 - RNA, 2160 nt, synthetic construct
  source 1..2160: mol_type = other RNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactggacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaaaagcctctccctgtctctgggtaaaatgttttgggtgctggtggtggttggtggagtcctggcttgctatagcttgctagtaacagtggcctttattattttctgggtgaggagtaagaggagcaggggcggccacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 82 - RNA, 2160 nt, synthetic construct
  source 1..2160: mol_type = other RNA
atggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcctggacgagagcaagtacggcccaccctgcccgccctgccccgccccagagttcctgggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgcggagcaagcggagccggggcggccacagcgactacatgaacatgaccccacggcggcccggccccacccggaagcactaccagccctacgccccaccacgggacttcgccgcctaccggagcaagaggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacgg

SEQ ID NO: 83 - RNA, 2160 nt, synthetic construct
  source 1..2160: mol_type = other RNA
atggagaccgacaccctgctactctgggtcttgctgctgtgggtcccgggttcgaccggtgatatcgtgctaacccagagcccggccatcctgtcagcaagtcccggggaaaaggtcactatgacctgcagggcttcctcctctgtcaactacatggattggtaccagaagaagcccgggtcctcccctaagccgtggatctacgcgacgagcaacctggcttctggagttcccgcccgcttcagcggctctggctcgggcacatcgtactccttaaccattagccgcgtcgaggctgaggatgctgctacctattactgtcagcagtggtctttcaaccctcccaccttcggcggcggcaccaagctggagatcaagggctccacaagcggaggaggatcgggcggtggcagcggtggcggcggctcctcggaggtgcagctgcagcagtcgggcgcggagctggtgaagccaggggcctccgtgaagatgtcatgcaaggcctccggctacacgttcaccagctacaacatgcattgggtgaagcagactcctggtcagggcctggagtggattggcgccatttacccaggcaatggggacaccagctacaaccagaaatttaaaggaaaggccactttgactgctgacaagagcagttccaccgcctacatgcagctgtcctcgcttacgtctgaagattctgcggactactactgtgctcgctctaattattacggcagcagctattggttcttcgacgtgtggggcgctggtacaactgtcactgtaagctctctcgacgagagcaagtacggtcccccatgcccaccctgtcctgcacccgagttcctgggtgggccctcagtgttcctgttcccccccaagcccaaggacacactcatgatttcgcgaactcctgaggtgacctgcgtggtcgtcgatgtgagccaggaggaccctgaggtccagttcaactggtatgtggacggggtggaggtgcacaacgcgaaaacaaagcctcgggaggaacagttcaacagtacgtaccgcgtggtatcagtgctgaccgttctccaccaggactggctgaacggtaaagagtacaagtgcaaggtgtccaacaagggtctgccctcctctatagagaagaccatctccaaagccaaggggcagccacgtgaacctcaggtgtacacccttcccccgtcacaggaggagatgaccaagaaccaggtttctctcacctgcctggtgaagggcttctacccctctgacatcgccgtggagtgggagtcgaatggtcagccggagaacaactacaaaactactccgccggtgctggacagcgacggctccttcttcctgtactcccgtctgaccgtggacaagtcccgctggcaagagggcaacgtgttctcgtgttctgtaatgcatgaggccttgcacaaccactacacccagaaatccctttctctgagtctgggcaaaatgttttgggtcctggtcgtggttggcggcgtgctggcttgttactccctcttggtgaccgtggcgttcatcatcttttgggtgcgctccaagcgcagcaggggaggccactccgactacatgaacatgacgccgcgcaggcccggcccaacccgcaagcactatcagccctatgcgccgcctcgggactttgccgcgtacaggagtaagcgcggccgcaagaagctgctgtacatcttcaagcagccgtttatgagaccggtgcagacgacccaggaggaggacgggtgctcttgtcgctttcccgaggaagaggagggcgggtgcgaacttcgtgtgaagttttcacgttcagcggacgctcctgcctaccaacagggacagaatcaattatacaacgagttgaacctgggacgacgcgaggagtacgatgtattggataaacgccggggccgcgaccccgagatgggcggtaaacctcgtcgcaagaatccacaggagggcctctacaacgagctgcagaaggacaagatggccgaggcctactccgagatcggtatgaagggcgagcgccgtcgcggaaaagggcatgacgggctttatcaggggttgagcaccgccaccaaagacacttatgacgcactgcacatgcaggccctcccccctaga

SEQ ID NO: 84 - protein, 1400 aa, synthetic construct
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 85 - protein, 23 aa, synthetic construct
MDYKDHDGDYKDHDIDYKDDDDK

SEQ ID NO: 86 - RNA, 4200 nt, synthetic construct
  source 1..4200: mol_type = other RNA
atggcccccaagaaaaagagaaaggtgggcattcacggcgtgcctgccgccgacaagaagtacagcatcggactggatatcggcaccaacagcgtgggctgggccgtgatcaccgacgagtacaaagtccctagcaaaaaattcaaggtgctgggcaacaccgaccggcactccatcaagaagaacctgatcggggctctgctttttgacagcggagaaaccgctgaggccacaagactgaaaagaaccgccagaaggaggtacacccgaagaaagaacagaatttgttacctgcaggagatcttttctaacgaaatggccaaggttgacgacagcttcttccacaggctggaagaatccttcctggtggaggaagataaaaagcacgagagacaccctatcttcggcaatatcgtggacgaggtcgcctaccacgagaagtaccccaccatctaccacctgagaaagaagctggtggattctacagataaggccgacctgagactcatctacctggccctggcccatatgatcaagttcagaggccacttcttaatcgaaggcgacctgaaccctgataacagcgacgtggataagctgttcatccagctggttcagacctacaaccagctatttgaggaaaaccctatcaacgcctctggcgtggacgcaaaagccatcctctccgcccggctgtccaagagcagaagactggaaaacctgattgctcagctgcctggcgaaaagaaaaacggcctgttcgggaacctgatcgctctgagcctgggcctgacccctaacttcaagagcaactttgatcttgctgaggacgccaagctgcagctgagcaaggatacctacgacgacgacctggataatctgctggcccaaatcggagatcagtacgccgatctcttcctggctgctaagaatctgagcgatgcaatcctgctgtctgacatcctgcgggtgaacaccgagatcacgaaggctccactgagcgccagtatgatcaagagatacgatgaacaccatcaggacctgaccctgctgaaggctctggtgcggcagcagctgccagaaaagtacaaggagattttcttcgaccagagcaaaaatggctatgccggctacatcgacggcggcgccagccaggaggagttctacaagttcatcaagcctatcctggagaagatggacggcaccgaggagctgctcgtgaagctgaacagagaggacctgctgcggaagcagcggaccttcgacaatggatctatcccccaccagatccacctgggcgagctgcatgccattctgagacggcaagaggatttttatcctttcctgaaagataacagagagaagatcgagaaaatcctgaccttcagaatcccttactacgtgggtcctctggcccgggggaacagccggttcgcctggatgaccagaaagagcgaggagacaataacaccttggaacttcgaagaggtggtggacaaaggggccagcgcccagagcttcatcgagcggatgacaaacttcgacaagaacctgcccaacgagaaggtgctgccaaagcacagcctgctgtatgaatacttcacagtgtacaacgaactgacaaaggtgaagtatgtgaccgagggaatgagaaagcccgcattcctgagcggcgaacaaaagaaggccatcgtggacctgctgtttaagaccaacagaaaggttaccgtgaagcagctgaaggaagattatttcaagaagatcgagtgcttcgattctgtggaaatcagcggcgtcgaagatagattcaacgccagcctgggcacctaccacgatctgctgaagatcatcaaggacaaagacttcctggacaacgaggagaacgaagacatcctggaagatatcgtgctgaccctgaccctgttcgaggaccgggaaatgatcgaggagcggctgaagacatacgcccacctgtttgatgacaaagtgatgaaacagctgaaaagacggagatacacaggctggggccgactgagcagaaagctgatcaacggaatccgggataagcagagcggcaagacaatcctggacttcctgaagtccgatggtttcgctaaccggaacttcatgcagttgatccacgacgacagcctgacctttaaggaagacattcagaaggcccaagtgtctggccagggcgacagtctgcatgagcacatcgccaacctggctggcagccctgctatcaagaagggcatcctgcagacagtgaaagtggtggacgagctggtgaaggttatgggccggcacaagccagagaatatcgtgatcgagatggcccgggagaatcagacaacccaaaagggccagaaaaactccagagaaagaatgaagagaatcgaggagggcatcaaggagctcggttctcagatcctgaaggagcaccccgtggaaaacacacagctgcagaacgagaagctgtacctgtactacctgcaaaatggcagagacatgtacgtagaccaggagctggatatcaatagactatctgattacgacgtggaccacatcgtgccccagagcttcctcaaagacgatagcatcgacaacaaggtgctgaccagaagcgacaagaaccggggcaagtctgataacgtgcccagcgaggaggtggtcaagaagatgaaaaattactggcggcaactgctgaatgctaagctgatcacccagagaaaattcgacaatctgacaaaggctgagagaggcggactgtctgagctggacaaggccggcttcatcaagagacagctggtggaaacccggcagatcaccaagcacgtggcccagatcctggacagccggatgaacaccaagtacgatgaaaacgacaagctgatcagagaggtcaaggttatcaccctgaaaagcaagctagtgtccgacttcagaaaagattttcagttctacaaggtgcgggaaattaacaactaccaccacgctcacgatgcttacctgaatgcagtggtgggcacagccctgatcaagaaataccccaagctggaatctgaatttgtgtacggcgattacaaggtgtacgacgtgcgcaaaatgatcgccaagagcgaacaggaaatcggcaaagccaccgccaaatatttcttttatagcaacatcatgaattttttcaaaaccgaaatcacactggccaacggcgagattagaaagcgccccctaatcgagacaaacggagaaacaggcgagatcgtttgggacaaaggcagagacttcgccaccgtgcggaaagtgttgagcatgcctcaggtgaacatcgtgaaaaagaccgaggtgcaaaccggaggcttcagcaaggagtccatcttgcctaagagaaactccgacaagctgatcgccagaaagaaggattgggaccccaagaaatacggcggcttcgattctcctacagtggcctattctgtgctggtggtcgccaaggtggaaaagggcaagtctaaaaagttgaagtccgtgaaggaactgctgggcatcacaatcatggaacggagcagctttgaaaaaaatcccatcgacttcctggaggccaaaggatataaggaggtgaagaaggacctgatcatcaaactgcctaagtacagcctgttcgaactcgaaaacggcagaaagagaatgctggcaagcgccggcgagctgcagaaaggaaacgagctggccctaccgagcaagtatgtgaacttcctgtacctggccagccactacgagaagcttaagggcagccctgaggataatgaacagaagcaactctttgtggaacaacacaaacactacctggacgaaatcatcgagcagataagcgagttcagtaagcgtgtgatcctggctgatgccaacctggacaaggtgctgagcgcctacaacaagcatagagataagcctatcagagaacaggccgaaaacatcatccacctgttcaccctgaccaatttgggcgcccctgccgccttcaagtacttcgacaccacaatcgatagaaaacgatacaccagcacaaaagaggtgctggatgccacactgattcaccagagcatcaccggtctgtacgagacacgtatcgacctgagccagctgggaggagacaaaagacctgccgccaccaagaaggccggccaagccaagaagaagaag

SEQ ID NO: 87 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 88 - protein, 249 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 89 - protein, 419 aa, synthetic construct
MRGLLVLSVLLGAVFGKEDFVGHQVLRISVADEAQVQKVKELEDLEHLQLDFWRGPAHPGSPIDVRVPFPSIQAVKIFLESHGISYETMIEDVQSLLDEEQEQMFAFRSRARSTDTFNYATYHTLEEIYDFLDLLVAENPHLVSKIQIGNTYEGRPIYVLKFSTGGSKRPAIWIDTGIHSREWVTQASGVWFAKKITQDYGQDAAFTAILDTLDIFLEIVTNPDGFAFTHSTNRMWRKTRSHTAGSLCIGVDPNRNWDAGFGLSGASSNPCSETYHGKFANSEVEVKSIVDFVKDHGNIKAFISIHSYSQLLMYPYGYKTEPVPDQDELDQLSKAAVTALASLYGTKFNYGSIIKAIYQASGSTIDWTYSQGIKYSFTFELRDTGRYGFLLPASQIIPTAKETWLALLTIMEHTLNHPY

SEQ ID NO: 90 - RNA, 1506 nt, synthetic construct
  source 1..1506: mol_type = other RNA
atggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcttcgtgcctgtgttcctgcctgccaagcctaccacaacacccgctcctagacctccaacaccagctccaacaatcgccagccagcctctgtctctgaggccagaagcttgtagacctgctgctggcggagccgtgcatacaagaggactggatttcgcctgcgacatctacatctgggctcctctggccggaacatgcggagtgctgctgctgagcctggtcatcaccctgtactgcaaccaccggaacagaagcaagcggagcaggctgctgcacagcgactacatgaacatgacccctagacggcccggacctaccagaaagcactaccagccttacgctcctcctcgggactttgccgcctatcggagcagagtgaagttcagcagatccgccgatgctcccgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 91 - RNA, 1506 nt, synthetic construct
  source 1..1506: mol_type = other RNA
atggccctgcccgttaccgccctgctcctgccgctggctctactgctgcacgccgctagacccgagatcgtgttaacgcagagccccgggacccttagcttgagcccgggagagagagccaccctgagctgcagagcttctcagagcgtgagcagcagctacctggcctggtatcagcagaagcccggccaagcccctagactgctgatctacggcgctagcagcagagccaccggcatccccgacagattctccggcagcggcagcggcaccgatttcaccctgaccatcagcagactggagcccgaggacttcgcggtgtactactgccaacaatacggcagcagcagattcaccttcggccccggcaccaaggtggacatcaaagggtccacctcgggcagcggcaagcctggaagcggagaaggctccaccaagggtcaagtgcagctggtgcagagcggcgccgaggtgaagaaacccggcagcagcgttaaggtgagctgcaaggacagcggcggcaccttcagcagctacgccatcagctgggtgagacaagcccccggccaaggcctggagtggatgggcggcatcatccccatcttcggcaccaccaactacgctcagcagttccaaggcagagtgaccatcaccgccgacgagagcacaagcaccgcctacatggagctgagcagcctgagatccgaggacaccgctgtctactattgcgcccgggaagccgtagccgccgattggctcgacccttggggccaaggcaccctggtgaccgtgagcagcttcgtgcccgtgtttctgcccgccaagccgaccaccacccccgcccctagacccccaacccccgcaccgacaatagcaagccaaccgctttcgctcagacctgaggcatgtaggcccgcggctgggggcgccgtgcacacaagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccctagaagacccggccccacaagaaagcactatcagccctacgccccccctagagacttcgccgcctacagaagcagagtgaagttcagcagaagcgccgacgcccccgcctatcagcaagggcagaatcagctttataacgagctgaaccttggcagaagagaggagtacgacgtgctggacaagagaagaggccgggaccccgagatgggcggcaagcctagaagaaagaacccccaagagggcctttacaacgagcttcagaaagacaagatggccgaggcctacagcgagatcggcatgaagggcgagagaagaagaggcaagggccacgacggcctgtaccaaggcctgagcaccgccaccaaggacacctacgacgccctgcacatgcaagccctgccccctaga

SEQ ID NO: 92 - RNA, 1506 nt, synthetic construct
  source 1..1506: mol_type = other RNA
atggcgctgccagtaacggcccttctgcttcctctcgccctcctcttgcatgcggcacgcccggaaattgttcttacacaaagtcctggtacgctgtctcttagccccggagaaagagcgacgttgagttgtagagcaagccaaagtgtatctagctcctacctcgcctggtaccaacagaagcccggacaggcgccccgcctgctcatctacggtgcgagtagtagggccacgggcattcctgatcgattctccggtagtggatcaggtactgattttactcttactatctcacggcttgagccggaagattttgcagtgtattactgccagcaatatgggagcagcaggttcacgttcggacctgggacaaaagtcgacatcaaaggcagcacatccggtagcggaaagcccggttcaggggagggctctacgaagggccaggtccaactcgtgcaatccggggcggaggtcaagaagccaggctctagtgtaaaagtcagttgtaaagacagcggcggcacttttagcagttatgctatcagttgggtccgacaggctccgggccagggtttggagtggatgggaggtatcataccgatattcgggactacaaactacgcgcaacaatttcagggtcgcgtgacgatcaccgccgatgagtccacgagcacggcttatatggaactgagctcactccggtctgaagacaccgcggtttattattgcgcacgcgaagcagtcgcagctgactggctggacccctggggacagggtaccctggttacggtctctagcttcgtccccgtgtttctgcctgctaaaccgacgacaactcctgcacctcgcccacccacaccagcaccgacgattgcatcccaacctctgtcacttcgaccagaggcttgcaggccggccgctggtggtgcggttcacacgcggggattggacttcgcttgcgatatttacatctgggcgccgctcgcggggacgtgtggagtacttctcctgtcccttgtaatcactttgtactgcaaccatcgcaatcgcagcaaacgcagtagacttcttcactctgattacatgaacatgacgccgcgacgaccgggtccaacgcggaagcactatcaaccttatgcacccccccgagacttcgctgcttaccgaagtcgggtaaaatttagtagatctgctgacgctcctgcgtatcaacagggacagaaccaactgtataacgagctgaatcttggccgacgcgaggagtatgacgtactcgataagcgccggggtagagaccccgagatgggcggcaagcccagacggaagaacccacaagagggattgtataacgagttgcaaaaagacaaaatggccgaggcctactccgaaataggtatgaagggcgaacgccgccgaggtaaaggtcacgacggactttaccagggactttctacggcaacgaaggacacatacgacgcgcttcatatgcaggcacttccaccaagg

SEQ ID NO: 93 - RNA, 1506 nt, synthetic construct
  source 1..1506: mol_type = other RNA
atggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccgg

SEQ ID NO: 94 - RNA, 1506 nt, synthetic construct
  source 1..1506: mol_type = other RNA
atggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtgcagctggtgcagtctggggctgaggtgaagaagcctgggtcctcggtgaaggtctcctgcaaggattctggaggcaccttcagcagctatgctatcagctgggtgcgacaggcccctggacaagggcttgagtggatgggagggatcatccctatctttggtacaacaaactacgcacagcagttccagggcagagtcacgattaccgcggacgaatccacgagcacagcctacatggagctgagcagcctgagatctgaggacacggccgtgtattactgtgcgagagaggccgtggccgccgactggctggacccctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 95 - RNA, 1506 nt, synthetic construct
  source 1..1506: mol_type = other RNA
atggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 96 - RNA, 1506 nt, synthetic construct
  source 1..1506: mol_type = other RNA
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 97 - RNA, 1455 nt, synthetic construct
  source 1..1455: mol_type = other RNA
atggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 98 - RNA, 1455 nt, synthetic construct
  source 1..1455: mol_type = other RNA
atggccctgcccgttaccgcactgcttctgccactggccctactactgcatgccgctagacccgagatagtgttaacgcagagccccggcacactgagcctgtcacccggcgagagagccaccctgagctgcagagcttctcagagcgtgagcagcagctacctggcctggtatcagcagaagcccggccaagcccctagactgctgatctacggcgctagcagcagagccaccggcatccccgacagattcagtgggagtggtagcggcaccgatttcaccctgaccatcagcagactggagcccgaggacttcgcggtgtactactgtcagcaatacggcagcagcagattcaccttcggccccggcaccaaggtcgacatcaaaggaagcacgtcgggttccggtaagcccggcagtggcgaaggatcgacaaagggccaagtgcagctggtgcagagcggcgccgaggtgaagaagcccggcagcagcgtgaaggtgagctgcaaggacagcggcggcaccttcagcagctacgccatcagctgggtgagacaagcccccggccaaggcctggagtggatgggcggcatcatccccatcttcggcaccaccaactacgctcagcagttccaaggcagagtgaccatcaccgccgacgagagcacaagcaccgcctacatggagctgagcagcctgagaagcgaggacaccgccgtgtactactgtgcccgggaagccgtggccgccgactggttagatccctggggccaaggcaccctggtgaccgtgagcagcatcgaggtgatgtacccccccccctacctggacaacgaaaagagcaacggcaccatcatccacgtgaagggcaagcacctgtgccctagccccctgttccccggcccaagcaaaccattctgggtgctggtggtggtgggcggcgtgctggcctgctacagcctgctggttacggtagctttcatcatcttctgggtgagaagcaagagaagcagactgctgcacagcgactacatgaacatgacccctagaagacccggccccacaagaaagcactatcagccctacgccccccctagagacttcgccgcctacagaagcagagtgaagttcagcagaagcgccgacgcccccgcctatcagcaagggcagaatcagctctacaacgagctgaacctgggcagaagagaggagtacgacgtgctggacaagagaagaggccgggaccccgagatgggcggcaagcctagaagaaagaacccccaagagggcctctacaacgaactgcaaaaggacaagatggccgaggcctacagcgagatcggcatgaagggcgagagaagaagaggcaagggccacgacggcctgtaccaaggcctgagcaccgccaccaaggacacctacgacgccctgcacatgcaagccctgccccctaga

SEQ ID NO: 99 - RNA, 1455 nt, synthetic construct
  source 1..1455: mol_type = other RNA
atggccctgcctgtgacggctcttctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacgg

SEQ ID NO: 100 - RNA, 1455 nt, synthetic construct
  source 1..1455: mol_type = other RNA
atggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacgg

SEQ ID NO: 101 - RNA, 1455 nt, synthetic construct
  source 1..1455: mol_type = other RNA
atggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccgg

SEQ ID NO: 102 - RNA, 1455 nt, synthetic construct
  source 1..1455: mol_type = other RNA
atggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtgcagctggtgcagtctggggctgaggtgaagaagcctgggtcctcggtgaaggtctcctgcaaggattctggaggcaccttcagcagctatgctatcagctgggtgcgacaggcccctggacaagggcttgagtggatgggagggatcatccctatctttggtacaacaaactacgcacagcagttccagggcagagtcacgattaccgcggacgaatccacgagcacagcctacatggagctgagcagcctgagatctgaggacacggccgtgtattactgtgcgagagaggccgtggccgccgactggctggacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 103 - RNA, 1455 nt, synthetic construct
  source 1..1455: mol_type = other RNA
atggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 104 - RNA, 1455 nt, synthetic construct
  source 1..1455: mol_type = other RNA
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcatcgaagtgatgtacccccctccctacctggacaacgagaagtccaacggcaccatcatccacgtgaagggcaagcacctgtgccccagccctctgtttcctggccctagcaagcccttctgggtgctggtggtcgtgggcggagtgctggcctgttacagcctgctcgtgacagtggccttcatcatcttttgggtgcgcagcaagcggagccggctgctgcacagcgactacatgaacatgacccccagacggccaggccccaccagaaagcactaccagccttacgcccctcccagagacttcgccgcctaccgcagcagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggacacgacggcctgtaccagggactgagcaccgccaccaaggacacctacgacgccctgcacatgcaggccctgccccccaga

SEQ ID NO: 105 - RNA, 2031 nt, synthetic construct
  source 1..2031: mol_type = other RNA
atggagacagacactttgctcttgtgggtactgctgctctgggtccctggaagtaccggggatattgtcttgacacaaagcccggcgatattgagtgcctccccaggtgaaaaagttactatgacatgccgagcatcttccagtgttaattatatggactggtaccagaagaaacccgggtcttcaccaaaaccttggatctacgccactagtaatctggcaagtggagtgcctgcccgcttcagcggcagtggcagcggtacttcctactcacttactatcagtcgagtagaggcagaggacgctgcgacctactactgtcagcagtggtcctttaatcctcccacattcggggggggtacaaagcttgaaataaagggttccactagtgggggtggtagtggaggcggaagcggtggaggaggaagctctgaggtgcagctccaacaatctggcgcggaactggtaaagcccggagcctccgttaaaatgagctgcaaagcaagcggctacacatttacctcttataacatgcattgggttaaacagacccctggacaaggacttgagtggatcggtgcgatatatccggggaacggggacactagctataaccagaaatttaagggcaaagccacattgacagcggacaagagcagttctacagcgtatatgcaactctcatccttgacctcagaagacagcgccgactattattgcgcaaggagcaattattacggtagctcatactggttctttgacgtgtggggggctggaactacggtaacagttagtagcgagagcaaatacggaccaccatgccccccctgccctgccccggagttcgacggcggaccgagcgtcttccttttcccccccaagcctaaagacactttgatgatcagtcggacaccggaagttacatgcgtagtagtggatgtctctcaggaagaccccgaggtgcaatttaattggtacgtcgatggtgtcgaagtacataatgcgaagacgaaaccccgggaggaacagttccagagcacgtatcgggtagtgagtgtcttgactgtgctgcatcaagattggctgaatggtaaggagtataaatgtaaagtcagcaacaaaggtctcccgtcatctatagaaaaaacgatatccaaggctaagggccaaccgagagagccacaggtgtataccctgcccccttcccaggaggaaatgacgaaaaatcaggtctctctcacgtgtttggtgaaagggttttatccatctgatattgccgtggagtgggaatctaatggacagcctgagaacaattacaagacaacgccaccggttctcgattccgatggtagtttctttctctattcacgcttgacagtcgataagtcccgctggcaagaaggcaacgttttctcctgttctgtcatgcacgaggccctgcacaatcattacacgcagaagtccttgagtttgagtttgggtaagatgttctgggtccttgttgttgtaggcggggttctcgcctgttattctctgctcgtgaccgtggcgtttataatcttttgggttaaacgaggccgcaaaaagttgctctacatcttcaaacaaccgtttatgaggccggtgcagactacacaggaagaggatggctgctcttgcagattcccggaagaggaggaaggtgggtgcgagttgagggtgaaattttccaggtccgccgatgctccagcttaccagcaagggcagaaccaattgtataacgagctgaatcttgggcggagggaggagtatgacgtgcttgacaaacgccggggtcgcgacccagaaatgggaggtaagccacgacggaaaaacccacaagagggtctttataacgaacttcaaaaggacaaaatggcggaggcctatagtgaaataggtatgaagggggagcggagacggggtaagggtcatgatggactctatcaaggtcttagcacagcgactaaggatacctacgatgcgctccatatgcaagctctgcctccgagg

SEQ ID NO: 106 - RNA, 2031 nt, synthetic construct
  source 1..2031: mol_type = other RNA
atggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcgagagcaagtacggcccaccctgcccgccctgccccgccccagagttcgacggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttccagagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgaagcggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacgg

SEQ ID NO: 107 - protein, 21 aa, synthetic construct
MALPVTALLLPLALLLHAARP

SEQ ID NO: 108 - protein, 20 aa, synthetic construct
METDTLLLWVLLLWVPGSTG

SEQ ID NO: 109 - protein, 22 aa, synthetic construct
MLLLVTSLLLCELPHPAFLLIP

SEQ ID NO: 110 - protein, 94 aa, synthetic construct
MRTLAILAAILLVALQAQAEPLQARADEVAAAPEQIAADIPEVVVSLAWDESLAPKHPGSRKNMDCYCRIPACIAGERRYGTCIYQGRLWAFCC

SEQ ID NO: 111 - RNA, 1926 nt, synthetic construct
  source 1..1926: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggcgctgccagtaacggcccttctgcttcctctcgccctcctcttgcatgcggcacgcccggaaattgttcttacacaaagtcctggtacgctgtctcttagccccggagaaagagcgacgttgagttgtagagcaagccaaagtgtatctagctcctacctcgcctggtaccaacagaagcccggacaggcgccccgcctgctcatctacggtgcgagtagtagggccacgggcattcctgatcgattctccggtagtggatcaggtactgattttactcttactatctcacggcttgagccggaagattttgcagtgtattactgccagcaatatgggagcagcaggttcacgttcggacctgggacaaaagtcgacatcaaaggcagcacatccggtagcggaaagcccggttcaggggagggctctacgaagggccaggtccaactcgtgcaatccggggcggaggtcaagaagccaggctctagtgtaaaagtcagttgtaaagacagcggcggcacttttagcagttatgctatcagttgggtccgacaggctccgggccagggtttggagtggatgggaggtatcataccgatattcgggactacaaactacgcgcaacaatttcagggtcgcgtgacgatcaccgccgatgagtccacgagcacggcttatatggaactgagctcactccggtctgaagacaccgcggtttattattgcgcacgcgaagcagtcgcagctgactggctggacccctggggacagggtaccctggttacggtctctagcttcgtccccgtgtttctgcctgctaaaccgacgacaactcctgcacctcgcccacccacaccagcaccgacgattgcatcccaacctctgtcacttcgaccagaggcttgcaggccggccgctggtggtgcggttcacacgcggggattggacttcgcttgcgatatttacatctgggcgccgctcgcggggacgtgtggagtacttctcctgtcccttgtaatcactttgtactgcaaccatcgcaatcgcagcaaacgcagtagacttcttcactctgattacatgaacatgacgccgcgacgaccgggtccaacgcggaagcactatcaaccttatgcacccccccgagacttcgctgcttaccgaagtcgggtaaaatttagtagatctgctgacgctcctgcgtatcaacagggacagaaccaactgtataacgagctgaatcttggccgacgcgaggagtatgacgtactcgataagcgccggggtagagaccccgagatgggcggcaagcccagacggaagaacccacaagagggattgtataacgagttgcaaaaagacaaaatggccgaggcctactccgaaataggtatgaagggcgaacgccgccgaggtaaaggtcacgacggactttaccagggactttctacggcaacgaaggacacatacgacgcgcttcatatgcaggcacttccaccaaggtgaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 112 - RNA, 1875 nt, synthetic construct
  source 1..1875: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcctgtgacggctcttctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 113 - RNA, 1770 nt, synthetic construct
  source 1..1770: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgagactactgttatttgaccaatgaattgacttctaataaaatctagtggtgatgctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 114 - RNA, 1771 nt, synthetic construct
  source 1..1771: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 115 - RNA, 1771 nt, synthetic construct; identical to SEQ ID NO: 114
  source 1..1771: mol_type = other RNA

SEQ ID NO: 116 - RNA, 1828 nt, synthetic construct
  source 1..1828: mol_type = other RNA
agcataaaagctcggtcctgggagggtttaaaagccagggggccgtctcgacctcagtctgaccttccctcccggcagcagcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctgaccctttgacacccttcttgtcctcctctctggccccatccaggcaaccaaataaagtttgagtgtaccaggaacagaatcctggggcttgctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - RNA, 1873 nt, synthetic construct
  source 1..1873: mol_type = other RNA
agcataaaagggctgctccttgctatagaagacctgggacagaggactgctgtctgccctctctggtcaccctgcctagctagaggatctgtgaccccagccgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgcttgcagaaaaagaaaaatgagctcaaaatttgctttgagagctacagggaattgctattactcctgtaccttctgctcaatttcctttcctcatctcaaataaatgccttgttacaagatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 118 - RNA, 1859 nt, synthetic construct
  source 1..1859: mol_type = other RNA
agcataaaagaactttcccccctcggcgccccaccggctcccgcgcgcctcccctcgcgcccgagcttcgagccaagcagcgtcctggggagcgcgtcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 119 - RNA, 1819 nt, synthetic construct
  source 1..1819: mol_type = other RNA
agcataaaagaaataagagagaaaagaagagtaagaagaaatataagaccccggcgccgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 120 - RNA, 1983 nt, synthetic construct
  source 1..1983: mol_type = other RNA
agcataaaagactagtattcttctggtccccacagactcagagagaacccgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 121 - RNA, 1825 nt, synthetic construct
  source 1..1825: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 122 - RNA, 1812 nt, synthetic construct
  source 1..1812: mol_type = other RNA
agcataaaagctagcttttctcttctgtcaaccccacacgcctttggcacagaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 123 - RNA, 1891 nt, synthetic construct
  source 1..1891: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 124 - RNA, 1950 nt, synthetic construct
  source 1..1950: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtgcagctggtgcagtctggggctgaggtgaagaagcctgggtcctcggtgaaggtctcctgcaaggattctggaggcaccttcagcagctatgctatcagctgggtgcgacaggcccctggacaagggcttgagtggatgggagggatcatccctatctttggtacaacaaactacgcacagcagttccagggcagagtcacgattaccgcggacgaatccacgagcacagcctacatggagctgagcagcctgagatctgaggacacggccgtgtattactgtgcgagagaggccgtggccgccgactggctggacccctggggccagggaaccctggtcaccgtctcctcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 125 - RNA, 1950 nt, synthetic construct
  source 1..1950: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 126 - RNA, 1950 nt, synthetic construct
  source 1..1950: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcccgttaccgccctgctcctgccgctggctctactgctgcacgccgctagacccgagatcgtgttaacgcagagccccgggacccttagcttgagcccgggagagagagccaccctgagctgcagagcttctcagagcgtgagcagcagctacctggcctggtatcagcagaagcccggccaagcccctagactgctgatctacggcgctagcagcagagccaccggcatccccgacagattctccggcagcggcagcggcaccgatttcaccctgaccatcagcagactggagcccgaggacttcgcggtgtactactgccaacaatacggcagcagcagattcaccttcggccccggcaccaaggtggacatcaaagggtccacctcgggcagcggcaagcctggaagcggagaaggctccaccaagggtcaagtgcagctggtgcagagcggcgccgaggtgaagaaacccggcagcagcgttaaggtgagctgcaaggacagcggcggcaccttcagcagctacgccatcagctgggtgagacaagcccccggccaaggcctggagtggatgggcggcatcatccccatcttcggcaccaccaactacgctcagcagttccaaggcagagtgaccatcaccgccgacgagagcacaagcaccgcctacatggagctgagcagcctgagatccgaggacaccgctgtctactattgcgcccgggaagccgtagccgccgattggctcgacccttggggccaaggcaccctggtgaccgtgagcagcttcgtgcccgtgtttctgcccgccaagccgaccaccacccccgcccctagacccccaacccccgcaccgacaatagcaagccaaccgctttcgctcagacctgaggcatgtaggcccgcggctgggggcgccgtgcacacaagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccctagaagacccggccccacaagaaagcactatcagccctacgccccccctagagacttcgccgcctacagaagcagagtgaagttcagcagaagcgccgacgcccccgcctatcagcaagggcagaatcagctttataacgagctgaaccttggcagaagagaggagtacgacgtgctggacaagagaagaggccgggaccccgagatgggcggcaagcctagaagaaagaacccccaagagggcctttacaacgagcttcagaaagacaagatggccgaggcctacagcgagatcggcatgaagggcgagagaagaagaggcaagggccacgacggcctgtaccaaggcctgagcaccgccaccaaggacacctacgacgccctgcacatgcaagccctgccccctagatgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 127 - RNA, 1950 nt, synthetic construct
  source 1..1950: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcttcgtgcctgtgttcctgcctgccaagcctaccacaacacccgctcctagacctccaacaccagctccaacaatcgccagccagcctctgtctctgaggccagaagcttgtagacctgctgctggcggagccgtgcatacaagaggactggatttcgcctgcgacatctacatctgggctcctctggccggaacatgcggagtgctgctgctgagcctggtcatcaccctgtactgcaaccaccggaacagaagcaagcggagcaggctgctgcacagcgactacatgaacatgacccctagacggcccggacctaccagaaagcactaccagccttacgctcctcctcgggactttgccgcctatcggagcagagtgaagttcagcagatccgccgatgctcccgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 128 - RNA, 1774 nt, synthetic construct
  source 1..1774: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 129 - RNA, 1761 nt, synthetic construct
  source 1..1761: mol_type = other RNA
agcataaaagctagcttttctcttctgtcaaccccacacgcctttggcacagaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 130 - RNA, 1840 nt, synthetic construct
  source 1..1840: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 131 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtgcagctggtgcagtctggggctgaggtgaagaagcctgggtcctcggtgaaggtctcctgcaaggattctggaggcaccttcagcagctatgctatcagctgggtgcgacaggcccctggacaagggcttgagtggatgggagggatcatccctatctttggtacaacaaactacgcacagcagttccagggcagagtcacgattaccgcggacgaatccacgagcacagcctacatggagctgagcagcctgagatctgaggacacggccgtgtattactgtgcgagagaggccgtggccgccgactggctggacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 132 - RNA, 1950 nt, synthetic construct
  source 1..1950: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 133 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 134 - RNA, 1950 nt, synthetic construct
  source 1..1950: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggcgctgccagtaacggcccttctgcttcctctcgccctcctcttgcatgcggcacgcccggaaattgttcttacacaaagtcctggtacgctgtctcttagccccggagaaagagcgacgttgagttgtagagcaagccaaagtgtatctagctcctacctcgcctggtaccaacagaagcccggacaggcgccccgcctgctcatctacggtgcgagtagtagggccacgggcattcctgatcgattctccggtagtggatcaggtactgattttactcttactatctcacggcttgagccggaagattttgcagtgtattactgccagcaatatgggagcagcaggttcacgttcggacctgggacaaaagtcgacatcaaaggcagcacatccggtagcggaaagcccggttcaggggagggctctacgaagggccaggtccaactcgtgcaatccggggcggaggtcaagaagccaggctctagtgtaaaagtcagttgtaaagacagcggcggcacttttagcagttatgctatcagttgggtccgacaggctccgggccagggtttggagtggatgggaggtatcataccgatattcgggactacaaactacgcgcaacaatttcagggtcgcgtgacgatcaccgccgatgagtccacgagcacggcttatatggaactgagctcactccggtctgaagacaccgcggtttattattgcgcacgcgaagcagtcgcagctgactggctggacccctggggacagggtaccctggttacggtctctagcttcgtccccgtgtttctgcctgctaaaccgacgacaactcctgcacctcgcccacccacaccagcaccgacgattgcatcccaacctctgtcacttcgaccagaggcttgcaggccggccgctggtggtgcggttcacacgcggggattggacttcgcttgcgatatttacatctgggcgccgctcgcggggacgtgtggagtacttctcctgtcccttgtaatcactttgtactgcaaccatcgcaatcgcagcaaacgcagtagacttcttcactctgattacatgaacatgacgccgcgacgaccgggtccaacgcggaagcactatcaaccttatgcacccccccgagacttcgctgcttaccgaagtcgggtaaaatttagtagatctgctgacgctcctgcgtatcaacagggacagaaccaactgtataacgagctgaatcttggccgacgcgaggagtatgacgtactcgataagcgccggggtagagaccccgagatgggcggcaagcccagacggaagaacccacaagagggattgtataacgagttgcaaaaagacaaaatggccgaggcctactccgaaataggtatgaagggcgaacgccgccgaggtaaaggtcacgacggactttaccagggactttctacggcaacgaaggacacatacgacgcgcttcatatgcaggcacttccaccaaggtgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 135 - RNA, 1902 nt, synthetic construct
  source 1..1902: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgatgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 136 - RNA, 1822 nt, synthetic construct
  source 1..1822: mol_type = other RNA
agcataaaagactcactatttgttttcgcgcccagttgcaaaaagtgtcgtaattgactaagaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 137 - RNA, 1822 nt, synthetic construct
  source 1..1822: mol_type = other RNA
agcataaaaggcttctagcaataggctataattcttcatcgtgttgtaacaagcgaactctgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcttcgtgcccgtgttcctgcccgccaagcccaccaccacaccagccccacggccacccaccccagccccaaccatcgccagccagcccctgagcctgcggcccgaggcctgccggcccgccgccggcggcgccgtgcacacccggggcctggacttcgcctgcgacatctacatctgggccccactggccggcacctgcggcgtgctcctgctgagcctggtgatcaccctgtactgcaaccaccggaaccggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 138 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 139 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 140 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcccgttaccgcactgcttctgccactggccctactactgcatgccgctagacccgagatagtgttaacgcagagccccggcacactgagcctgtcacccggcgagagagccaccctgagctgcagagcttctcagagcgtgagcagcagctacctggcctggtatcagcagaagcccggccaagcccctagactgctgatctacggcgctagcagcagagccaccggcatccccgacagattcagtgggagtggtagcggcaccgatttcaccctgaccatcagcagactggagcccgaggacttcgcggtgtactactgtcagcaatacggcagcagcagattcaccttcggccccggcaccaaggtcgacatcaaaggaagcacgtcgggttccggtaagcccggcagtggcgaaggatcgacaaagggccaagtgcagctggtgcagagcggcgccgaggtgaagaagcccggcagcagcgtgaaggtgagctgcaaggacagcggcggcaccttcagcagctacgccatcagctgggtgagacaagcccccggccaaggcctggagtggatgggcggcatcatccccatcttcggcaccaccaactacgctcagcagttccaaggcagagtgaccatcaccgccgacgagagcacaagcaccgcctacatggagctgagcagcctgagaagcgaggacaccgccgtgtactactgtgcccgggaagccgtggccgccgactggttagatccctggggccaaggcaccctggtgaccgtgagcagcatcgaggtgatgtacccccccccctacctggacaacgaaaagagcaacggcaccatcatccacgtgaagggcaagcacctgtgccctagccccctgttccccggcccaagcaaaccattctgggtgctggtggtggtgggcggcgtgctggcctgctacagcctgctggttacggtagctttcatcatcttctgggtgagaagcaagagaagcagactgctgcacagcgactacatgaacatgacccctagaagacccggccccacaagaaagcactatcagccctacgccccccctagagacttcgccgcctacagaagcagagtgaagttcagcagaagcgccgacgcccccgcctatcagcaagggcagaatcagctctacaacgagctgaacctgggcagaagagaggagtacgacgtgctggacaagagaagaggccgggaccccgagatgggcggcaagcctagaagaaagaacccccaagagggcctctacaacgaactgcaaaaggacaagatggccgaggcctacagcgagatcggcatgaagggcgagagaagaagaggcaagggccacgacggcctgtaccaaggcctgagcaccgccaccaaggacacctacgacgccctgcacatgcaagccctgccccctagatgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 141 - RNA, 1774 nt, synthetic construct
  source 1..1774: mol_type = other RNA
agcataaaagtcacatttgcttctgacatagttgtgttgactcacaaccccagaaacagacatcgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 142 - RNA, 1774 nt, synthetic construct
  source 1..1774: mol_type = other RNA
agcataaaagtcacatttgcttctgacatagttgtgttgactcacaaccccagaaacagacatcgaattcgctgccaccatggccctgcccgttaccgcactgcttctgccactggccctactactgcatgccgctagacccgagatagtgttaacgcagagccccggcacactgagcctgtcacccggcgagagagccaccctgagctgcagagcttctcagagcgtgagcagcagctacctggcctggtatcagcagaagcccggccaagcccctagactgctgatctacggcgctagcagcagagccaccggcatccccgacagattcagtgggagtggtagcggcaccgatttcaccctgaccatcagcagactggagcccgaggacttcgcggtgtactactgtcagcaatacggcagcagcagattcaccttcggccccggcaccaaggtcgacatcaaaggaagcacgtcgggttccggtaagcccggcagtggcgaaggatcgacaaagggccaagtgcagctggtgcagagcggcgccgaggtgaagaagcccggcagcagcgtgaaggtgagctgcaaggacagcggcggcaccttcagcagctacgccatcagctgggtgagacaagcccccggccaaggcctggagtggatgggcggcatcatccccatcttcggcaccaccaactacgctcagcagttccaaggcagagtgaccatcaccgccgacgagagcacaagcaccgcctacatggagctgagcagcctgagaagcgaggacaccgccgtgtactactgtgcccgggaagccgtggccgccgactggttagatccctggggccaaggcaccctggtgaccgtgagcagcatcgaggtgatgtacccccccccctacctggacaacgaaaagagcaacggcaccatcatccacgtgaagggcaagcacctgtgccctagccccctgttccccggcccaagcaaaccattctgggtgctggtggtggtgggcggcgtgctggcctgctacagcctgctggttacggtagctttcatcatcttctgggtgagaagcaagagaagcagactgctgcacagcgactacatgaacatgacccctagaagacccggccccacaagaaagcactatcagccctacgccccccctagagacttcgccgcctacagaagcagagtgaagttcagcagaagcgccgacgcccccgcctatcagcaagggcagaatcagctctacaacgagctgaacctgggcagaagagaggagtacgacgtgctggacaagagaagaggccgggaccccgagatgggcggcaagcctagaagaaagaacccccaagagggcctctacaacgaactgcaaaaggacaagatggccgaggcctacagcgagatcggcatgaagggcgagagaagaagaggcaagggccacgacggcctgtaccaaggcctgagcaccgccaccaaggacacctacgacgccctgcacatgcaagccctgccccctagatgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 143 - RNA, 1768 nt, synthetic construct
  source 1..1768: mol_type = other RNA
agcataaaagaaataagagagaaaagaagagtaagaagaaatataagaccccggcgccgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 144 - RNA, 1932 nt, synthetic construct
  source 1..1932: mol_type = other RNA
agcataaaagactagtattcttctggtccccacagactcagagagaacccgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 145 - RNA, 1822 nt, synthetic construct
  source 1..1822: mol_type = other RNA
agcataaaagggctgctccttgctatagaagacctgggacagaggactgctgtctgccctctctggtcaccctgcctagctagaggatctgtgaccccagccgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgcttgcagaaaaagaaaaatgagctcaaaatttgctttgagagctacagggaattgctattactcctgtaccttctgctcaatttcctttcctcatctcaaataaatgccttgttacaagatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 146 - RNA, 1777 nt, synthetic construct
  source 1..1777: mol_type = other RNA
agcataaaagctcggtcctgggagggtttaaaagccagggggccgtctcgacctcagtctgaccttccctcccggcagcagcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctgaccctttgacacccttcttgtcctcctctctggccccatccaggcaaccaaataaagtttgagtgtaccaggaacagaatcctggggcttgctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 147 - RNA, 1808 nt, synthetic construct
  source 1..1808: mol_type = other RNA
agcataaaagaactttcccccctcggcgccccaccggctcccgcgcgcctcccctcgcgcccgagcttcgagccaagcagcgtcctggggagcgcgtcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 148 - RNA, 1771 nt, synthetic construct
  source 1..1771: mol_type = other RNA
agcataaaagactcactatttgttttcgcgcccagttgcaaaaagtgtcgtaattgactaagaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 149 - RNA, 1771 nt, synthetic construct
  source 1..1771: mol_type = other RNA
agcataaaaggcttctagcaataggctataattcttcatcgtgttgtaacaagcgaactctgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 150 - RNA, 1720 nt, synthetic construct
  source 1..1720: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 151 - RNA, 1776 nt, synthetic construct
  source 1..1776: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 152 - RNA, 1822 nt, synthetic construct
  source 1..1822: mol_type = other RNA
agcataaaagggctgctccttgctatagaagacctgggacagaggactgctgtctgccctctctggtcaccctgcctagctagaggatctgtgaccccagccgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccgcttgcagaaaaagaaaaatgagctcaaaatttgctttgagagctacagggaattgctattactcctgtaccttctgctcaatttcctttcctcatctcaaataaatgccttgttacaagatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 153 - RNA, 1777 nt, synthetic construct
  source 1..1777: mol_type = other RNA
agcataaaagctcggtcctgggagggtttaaaagccagggggccgtctcgacctcagtctgaccttccctcccggcagcagcgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccgctgaccctttgacacccttcttgtcctcctctctggccccatccaggcaaccaaataaagtttgagtgtaccaggaacagaatcctggggcttgctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 154 - RNA, 1808 nt, synthetic construct
  source 1..1808: mol_type = other RNA
agcataaaagaactttcccccctcggcgccccaccggctcccgcgcgcctcccctcgcgcccgagcttcgagccaagcagcgtcctggggagcgcgtcgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 155 - RNA, 1771 nt, synthetic construct
  source 1..1771: mol_type = other RNA
agcataaaagactcactatttgttttcgcgcccagttgcaaaaagtgtcgtaattgactaagaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 156 - RNA, 1771 nt, synthetic construct
  source 1..1771: mol_type = other RNA
agcataaaaggcttctagcaataggctataattcttcatcgtgttgtaacaagcgaactctgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 157 - RNA, 1720 nt, synthetic construct
  source 1..1720: mol_type = other RNA
agcataaaagtcacatttgcttctgacatagttgtgttgactcacaaccccagaaacagacatcgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 158 - RNA, 1776 nt, synthetic construct
  source 1..1776: mol_type = other RNA
agcataaaagtcacatttgcttctgacatagttgtgttgactcacaaccccagaaacagacatcgaattcgctgccaccatggctctgcctgtgacagctctgctgctgcctctggctctgcttctgcatgccgccagacctgagatcgtgctgactcagtctcccggcacactgtcactgtctccaggcgaaagagccacactgagctgtagagccagccagagcgtgtccagctcttacctggcttggtatcagcagaagcccggacaggctcccagactgctgatctatggcgcctctagcagagctacaggcatccccgatagattcagcggcagcggctctggcaccgatttcaccctgaccatcagcagactggaacccgaggacttcgccgtgtactactgtcagcagtacggcagcagccggttcacatttggccctggcacaaaggtggacatcaagggcagcacatctggcagcggcaaacctggatctggcgagggctctacaaaaggccaggtgcagctggttcagtctggcgccgaagtgaagaaacctggcagcagcgtgaaggtgtcctgcaaggatagcggcggcacctttagcagctacgccatctcttgggtccgacaggcacctggacaaggcctggaatggatgggcggcatcatccctatcttcggcaccaccaattacgcccagcagttccagggcagagtgaccatcacagccgatgagagcaccagcaccgcctacatggaactgagcagcctgagaagcgaggacacagccgtgtattattgcgccagagaagccgtggccgccgattggcttgatccttggggacagggaaccctggtcaccgtgtctagcatcgaagtgatgtaccctccaccttacctcgacaacgagaagtccaacggcaccatcatccacgtgaagggcaagcacctgtgtccttctccactgttccccggacctagcaagcctttctgggtgctcgttgttgttggcggcgtgctggcctgttactctctgctggttaccgtggccttcatcatcttttgggtccgcagcaagcggagcagactcctgcacagcgactacatgaacatgacccctagacggcccggaccaaccagaaagcactaccagccttacgctcctcctcgggatttcgccgcctacagatccagagtgaagttcagcagatccgccgacgctcctgcctatcagcagggacagaatcagctgtataacgagctgaacctggggcgcagagaagagtacgacgtgctggacaagagaagaggcagggaccctgagatgggcggaaagcccagacggaagaatcctcaagagggcctgtataatgaactgcagaaagacaagatggccgaggcctacagcgagatcggaatgaagggcgaacgcagaagaggaaagggccacgacggactgtatcagggcctgagcacagccaccaaggacacctatgatgccctgcacatgcaggccctgcctccaagatgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 159 - RNA, 1774 nt, synthetic construct
  source 1..1774: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 160 - RNA, 1822 nt, synthetic construct
  source 1..1822: mol_type = other RNA
agcataaaagggctgctccttgctatagaagacctgggacagaggactgctgtctgccctctctggtcaccctgcctagctagaggatctgtgaccccagccgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgcttgcagaaaaagaaaaatgagctcaaaatttgctttgagagctacagggaattgctattactcctgtaccttctgctcaatttcctttcctcatctcaaataaatgccttgttacaagatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 161 - RNA, 1777 nt, synthetic construct
  source 1..1777: mol_type = other RNA
agcataaaagctcggtcctgggagggtttaaaagccagggggccgtctcgacctcagtctgaccttccctcccggcagcagcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgctgaccctttgacacccttcttgtcctcctctctggccccatccaggcaaccaaataaagtttgagtgtaccaggaacagaatcctggggcttgctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 162 - RNA, 1808 nt, synthetic construct
  source 1..1808: mol_type = other RNA
agcataaaagaactttcccccctcggcgccccaccggctcccgcgcgcctcccctcgcgcccgagcttcgagccaagcagcgtcctggggagcgcgtcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 163 - RNA, 1771 nt, synthetic construct
  source 1..1771: mol_type = other RNA
agcataaaagactcactatttgttttcgcgcccagttgcaaaaagtgtcgtaattgactaagaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 164 - RNA, 1771 nt, synthetic construct
  source 1..1771: mol_type = other RNA
agcataaaaggcttctagcaataggctataattcttcatcgtgttgtaacaagcgaactctgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 165 - RNA, 1720 nt, synthetic construct
  source 1..1720: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 166 - RNA, 1776 nt, synthetic construct
  source 1..1776: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacgattcactttcggccctgggaccaaagtggatatcaaaggcagcacctccggcagcggcaagcctggctctggcgagggctctaccaagggccaggtccagctggtgcagtctggggctgaggtgaagaagcctgggtcctcggtgaaggtctcctgcaaggactctggaggcaccttcagcagctatgctatcagctgggtgcgacaggcccctggacaaggacttgagtggatgggagggatcatccctatctttggtacaacaaactacgcacagcagttccagggcagagtcacgattaccgcggacgaatccacgagcacagcctacatggagctgagcagtctgagatctgaggacacggccgtgtattactgtgcgagagaagcagtagctgcggactggttagacccctggggccagggaaccctggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 167 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcatcgaagtgatgtacccccctccctacctggacaacgagaagtccaacggcaccatcatccacgtgaagggcaagcacctgtgccccagccctctgtttcctggccctagcaagcccttctgggtgctggtggtcgtgggcggagtgctggcctgttacagcctgctcgtgacagtggccttcatcatcttttgggtgcgcagcaagcggagccggctgctgcacagcgactacatgaacatgacccccagacggccaggccccaccagaaagcactaccagccttacgcccctcccagagacttcgccgcctaccgcagcagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggacacgacggcctgtaccagggactgagcaccgccaccaaggacacctacgacgccctgcacatgcaggccctgccccccagatgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 168 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 169 - RNA, 1774 nt, synthetic construct
  source 1..1774: mol_type = other RNA
agcataaaagtcacatttgcttctgacatagttgtgttgactcacaaccccagaaacagacatcgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 170 - RNA, 1822 nt, synthetic construct
  source 1..1822: mol_type = other RNA
agcataaaagggctgctccttgctatagaagacctgggacagaggactgctgtctgccctctctggtcaccctgcctagctagaggatctgtgaccccagccgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccgcttgcagaaaaagaaaaatgagctcaaaatttgctttgagagctacagggaattgctattactcctgtaccttctgctcaatttcctttcctcatctcaaataaatgccttgttacaagatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 171 - RNA, 1777 nt, synthetic construct
  source 1..1777: mol_type = other RNA
agcataaaagctcggtcctgggagggtttaaaagccagggggccgtctcgacctcagtctgaccttccctcccggcagcagcgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccgctgaccctttgacacccttcttgtcctcctctctggccccatccaggcaaccaaataaagtttgagtgtaccaggaacagaatcctggggcttgctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 172 - RNA, 1808 nt, synthetic construct
  source 1..1808: mol_type = other RNA
agcataaaagaactttcccccctcggcgccccaccggctcccgcgcgcctcccctcgcgcccgagcttcgagccaagcagcgtcctggggagcgcgtcgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 173 - RNA, 1771 nt, synthetic construct
  source 1..1771: mol_type = other RNA
agcataaaagactcactatttgttttcgcgcccagttgcaaaaagtgtcgtaattgactaagaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 174 - RNA, 1771 nt, synthetic construct
  source 1..1771: mol_type = other RNA
agcataaaaggcttctagcaataggctataattcttcatcgtgttgtaacaagcgaactctgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 175 - RNA, 1720 nt, synthetic construct
  source 1..1720: mol_type = other RNA
agcataaaagtcacatttgcttctgacatagttgtgttgactcacaaccccagaaacagacatcgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 176 - RNA, 1719 nt, synthetic construct
  source 1..1719: mol_type = other RNA
agcataaaagtcacatttgcttctgacatagttgtgttgactcacaaccccagaaacagacatcgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccgagactactgttatttgaccaatgaattgacttctaataaaatctagtggtgatgctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 177 - RNA, 2476 nt, synthetic construct
  source 1..2476: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggagacagacactttgctcttgtgggtactgctgctctgggtccctggaagtaccggggatattgtcttgacacaaagcccggcgatattgagtgcctccccaggtgaaaaagttactatgacatgccgagcatcttccagtgttaattatatggactggtaccagaagaaacccgggtcttcaccaaaaccttggatctacgccactagtaatctggcaagtggagtgcctgcccgcttcagcggcagtggcagcggtacttcctactcacttactatcagtcgagtagaggcagaggacgctgcgacctactactgtcagcagtggtcctttaatcctcccacattcggggggggtacaaagcttgaaataaagggttccactagtgggggtggtagtggaggcggaagcggtggaggaggaagctctgaggtgcagctccaacaatctggcgcggaactggtaaagcccggagcctccgttaaaatgagctgcaaagcaagcggctacacatttacctcttataacatgcattgggttaaacagacccctggacaaggacttgagtggatcggtgcgatatatccggggaacggggacactagctataaccagaaatttaagggcaaagccacattgacagcggacaagagcagttctacagcgtatatgcaactctcatccttgacctcagaagacagcgccgactattattgcgcaaggagcaattattacggtagctcatactggttctttgacgtgtggggggctggaactacggtaacagttagtagcgagagcaaatacggaccaccatgccccccctgccctgccccggagttcgacggcggaccgagcgtcttccttttcccccccaagcctaaagacactttgatgatcagtcggacaccggaagttacatgcgtagtagtggatgtctctcaggaagaccccgaggtgcaatttaattggtacgtcgatggtgtcgaagtacataatgcgaagacgaaaccccgggaggaacagttccagagcacgtatcgggtagtgagtgtcttgactgtgctgcatcaagattggctgaatggtaaggagtataaatgtaaagtcagcaacaaaggtctcccgtcatctatagaaaaaacgatatccaaggctaagggccaaccgagagagccacaggtgtataccctgcccccttcccaggaggaaatgacgaaaaatcaggtctctctcacgtgtttggtgaaagggttttatccatctgatattgccgtggagtgggaatctaatggacagcctgagaacaattacaagacaacgccaccggttctcgattccgatggtagtttctttctctattcacgcttgacagtcgataagtcccgctggcaagaaggcaacgttttctcctgttctgtcatgcacgaggccctgcacaatcattacacgcagaagtccttgagtttgagtttgggtaagatgttctgggtccttgttgttgtaggcggggttctcgcctgttattctctgctcgtgaccgtggcgtttataatcttttgggttaaacgaggccgcaaaaagttgctctacatcttcaaacaaccgtttatgaggccggtgcagactacacaggaagaggatggctgctcttgcagattcccggaagaggaggaaggtgggtgcgagttgagggtgaaattttccaggtccgccgatgctccagcttaccagcaagggcagaaccaattgtataacgagctgaatcttgggcggagggaggagtatgacgtgcttgacaaacgccggggtcgcgacccagaaatgggaggtaagccacgacggaaaaacccacaagagggtctttataacgaacttcaaaaggacaaaatggcggaggcctatagtgaaataggtatgaagggggagcggagacggggtaagggtcatgatggactctatcaaggtcttagcacagcgactaaggatacctacgatgcgctccatatgcaagctctgcctccgaggtgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 178 - RNA, 2352 nt, synthetic construct
  source 1..2352: mol_type = other RNA
agcataaaagtcacatttgcttctgacatagttgtgttgactcacaaccccagaaacagacatcgaattcgctgccaccatggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcgagagcaagtacggcccaccctgcccgccctgccccgccccagagttcgacggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttccagagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgaagcggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 179 - RNA, 1128 nt, synthetic construct
  source 1..1128: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggtgtccaagggcgaggaagataatatggccatcatcaaggagttcatgaggttcaaggtgcacatggaaggcagcgttaacggccacgagttcgagatcgagggcgaaggagaaggaagaccctacgagggaacacagaccgctaaactgaaagtgacaaagggaggccctctgccattcgcctgggacatcctgtcccctcagttcatgtacggcagcaaggcctacgtcaagcatcctgccgacatccctgactacctgaagctgagctttcccgagggcttcaagtgggaacgggtgatgaacttcgaggacggcggcgtggtgaccgtgacacaagacagcagcctgcaggatggagagtttatttataaggtgaagctgagaggcaccaacttccccagcgacggccctgtgatgcagaaaaagaccatgggctgggaggcctctagcgagcggatgtatcctgaagatggcgccctgaagggcgagatcaagcagagactgaaactgaaggacggcggccactacgacgccgaggtgaaaaccacctacaaggccaagaagcctgtgcagctgcccggcgcttacaacgtgaatatcaagctcgatatcaccagccacaacgaggattacaccatcgtggaacagtacgagagagccgaaggcagacactctacaggaggcatggacgagctgtacaagtgaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 180 - protein, 121 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 181 - protein, 10 aa, synthetic construct
RYTFTDYNLH

SEQ ID NO: 182 - protein, 10 aa, synthetic construct
FIYPYNGGTG

SEQ ID NO: 183 - protein, 12 aa, synthetic construct
DHRYNEGVSFDY

SEQ ID NO: 184 - protein, 111 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIK

SEQ ID NO: 185 - protein, 15 aa, synthetic construct
RASESVDGFGNSFMN

SEQ ID NO: 186 - protein, 7 aa, synthetic construct
LASNLES

SEQ ID NO: 187 - protein, 9 aa, synthetic construct
QQNNEDPYT

SEQ ID NO: 188 - protein, 109 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWGQGTTVTVSS

SEQ ID NO: 189 - protein, 121 aa, synthetic construct
QVQLVQSGAEVKKPGASVKVSCKASRYTFTDYNLHWVRQAPGQGLEWMGFIYPYNGGTGYAQKFQGRVTMTRDTSTSTVYMELSSLRSEDTAVYYCARDHRYNEGVSFDYWGQGTTVTVSS

SEQ ID NO: 190 - protein, 121 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSS

SEQ ID NO: 191 - protein, 121 aa, synthetic construct
QVQLVQSGAEVKKPGASVKISCKASRYTFTDYNLHWVRQAPGQGLEWIGFIYPYNGGTGYAQKFQGRATLTVDTSTSTAYMELSSLRSEDTAVYYCARDHRYNEGVSFDYWGQGTTLTVSS

SEQ ID NO: 192 - protein, 121 aa, synthetic construct
QVQLVQSGAEVKKPGASVKVSCKASRYTFTDYNLHWVRQAPGQGLEWMGFIYPYNGGTGYAQKFQGRVTMTVDYSTSTAYMELSSLRSEDTAVYYCARDHRYNEGVSFDYWGQGTTVTVSS

SEQ ID NO: 193 - protein, 121 aa, synthetic construct
QVQLVQSGAEVKKPGASVKISCKASRYTFTDYNLHWVRQAPGQGLEWIGFIYPYNGGTGYAQKFQGRATLTVDYSTSTAYMELSSLRSEDTAVYYCARDHRYNEGVSFDYWGQGTTLTVSS

SEQ ID NO: 194 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPFGQGTKLEIK

SEQ ID NO: 195 - protein, 111 aa, synthetic construct
DIQMTQSPSSLSASVGDRVTITCRASESVDGFGNSFMNWYQQKPGKAPKLLIYLASNLESGVPSRFSGSGSGTDFTLTISSLQPEDFATYYCQQNNEDPYTFGQGTKLEIK

SEQ ID NO: 196 - protein, 111 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIK

SEQ ID NO: 197 - protein, 111 aa, synthetic construct
DIQLTQSPSSLSASVGDRATITCRASESVDGFGNSFMNWYQQKPGKAPKLLIYLASNLESGVPSRFSGSGSRTDFTLTISSVQPEDFATYYCQQNNEDPYTFGQGTKLEIK

SEQ ID NO: 198 - protein, 118 aa, synthetic construct [sequence omitted]

SEQ ID NO: 199 - protein, 107 aa, synthetic construct
DVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIK

SEQ ID NO: 200 - protein, 118 aa, synthetic construct
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSS

SEQ ID NO: 201 - protein, 107 aa, synthetic construct
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELK

SEQ ID NO: 202 - protein, 121 aa, synthetic construct
QVQLVQSGAEVKKPGASVKVSCKASRYTFTDYNLHWVRQAPGQGLEWMGFIYPYSGGTGYAQKFQGRVTMTVDTSTSTAYMELSSLRSEDTAVYYCARDHRYNEGVSFDYWGQGTTVTVSS

SEQ ID NO: 203 - protein, 121 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSS

SEQ ID NO: 204 - protein, 121 aa, unidentified
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSS

SEQ ID NO: 205 - protein, 121 aa, synthetic construct
QVQLVQSGAEVKKPGASVKVSCKASRYTFTDYNLHWVRQAPGQGLEWMGFIYPYDGGTGYAQKFQGRVTMTVDTSTSTAYMELSSLRSEDTAVYYCARDHRYNEGVSFDYWGQGTTVTVSS

SEQ ID NO: 206 - protein, 108 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWGQGTLVTVS

SEQ ID NO: 207 - protein, 102 aa, synthetic construct
QVQLVQSGAEVKKPGASVKVSCKASRYTFTDYNLHWVRQAPGQGLEWMGFIYPYNGGTNYAQKFQGRVTITADTSTSTAYMELSSLRSEDTAVYYCARDHRY

SEQ ID NO: 208 - protein, 121 aa, synthetic construct
QVQLVQSGAEVKKPGASVKVSCKASRYTFTDYNLHWVRQAPGQGLEWIGFIYPYNGGTGYAQKFQGRVTITADTSTSTAYMELSSLRSEDTAVYYCARDHRYNEGVSFDYWGQGTLVTVSS

SEQ ID NO: 209 - protein, 121 aa, synthetic construct
QVQLVQSGAEVKKPGASVKVSCKASRYTFTDYNLHWVRQAPGQGLEWIGFIYPYNGGTGYAQKFKGRVTITADTSTSTAYMELSSLRSEDTAVYYCARDHRYNEGVSFDYWGQGTLVTVSS

SEQ ID NO: 210 - protein, 121 aa, synthetic construct
QVQLVQSGAEVKKPGASVKVSCKASRYTFTDYNLHWVRQAPGQGLEWMGFIYPYNGGTGYAQKFQGRVTLTVDYSTSTAYMELSSLRSEDTAVYYCARDHRYNEGVSFDYWGQGTLVTVSS

SEQ ID NO: 211 - protein, 121 aa, synthetic construct
QVQLVQSGAEVKKPGASVKVSCKASRYTFTDYNLHWVRQAPGQGLEWIGFIYPYNGGTGYAQKFQGRVTLTVDYSTSTAYMELSSLRSEDTAVYYCARDHRYNEGVSFDYWGQGTLVTVSS

SEQ ID NO: 212 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 213 - protein, 111 aa, synthetic construct
EIVLTQSPGTLSLSPGERATLSCRASESVDGFGNSFMNWYQQKPGQAPRLLIYLASNLESGIPARFSGSGSGTDFTLTISRLEPEDFAVYYCQQNNEDPYTFGQGTKVEIK

SEQ ID NO: 214 - protein, 111 aa, synthetic construct
EIVLTQSPGTLSLSPGERATLSCRASESVDGFGNSFMNWYQQKPGQAPRLLIYLASNLESGVPARFSGSGSGTDFTLTISRLEPEDFAVYYCQQNNEDPYTFGQGTKVEIK

SEQ ID NO: 215 - protein, 111 aa, synthetic construct
EIVLTQSPGTLSLSPGERATLSCRASESVDGFGNSFMNWYQQKPGQAPRLLIYLASNLESGVPARFSGSGSRTDFTLTISRLEPEDFAVYYCQQNNEDPYTFGQGTKVEIK

SEQ ID NO: 216 - protein, 107 aa, Homo sapiens [sequence omitted]

SEQ ID NO: 217 - protein, 330 aa, Homo sapiens [sequence omitted]

SEQ ID NO: 218 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 330 aa, synthetic construct [sequence omitted]

SEQ ID NO: 220 - protein, 10 aa, synthetic construct
  VARIANT 8: note = X is N, S, Q, or A
RYTFTDYXLH

SEQ ID NO: 221 - protein, 10 aa, synthetic construct
  VARIANT 6: note = X is N, S, Q, or A
FIYPYXGGTG

SEQ ID NO: 222 - protein, 10 aa, synthetic construct
  VARIANT 6: note = X is N, Q, D, S, or A
FIYPYXGGTG

SEQ ID NO: 223 - protein, 12 aa, synthetic construct
  VARIANT 5: note = X is N, S, Q, or A
DHRYXEGVSFDY

SEQ ID NO: 224 - protein, 15 aa, synthetic construct
  VARIANT 7: note = X is D, E, S, or A
  VARIANT 11: note = X is N, S, Q, or A
RASESVXGFGXSFMN

SEQ ID NO: 225 - protein, 7 aa, synthetic construct
  VARIANT 4: note = X is N, Q, D, S, or A
LASXLES

SEQ ID NO: 226 - protein, 9 aa, synthetic construct
  VARIANT 3: note = X is N, Q, D, S, or A
  VARIANT 4: note = X is N, Q, D, S, or A
  VARIANT 6: note = X is D, E, S, or A
QQXXEXPYT

SEQ ID NO: 227 - protein, 55 aa, unidentified
KNRKAKAKPVTRGAGAGGRQRGQNKERPPPVPNPDYEPIRKGQRDLYSGLNQRRI

SEQ ID NO: 228 - protein, 25 aa, unidentified
KTNIRSSTRDWKDHKFKWRKDPQDK

SEQ ID NO: 229 - protein, 18 aa, synthetic construct
GSTSGGGSGGGSGGGGSS

SEQ ID NO: 230 - protein, 45 aa, unidentified
TTTPAPRPPTPAPTIASQPLSLRPEACRPAAGGAVHTRGLDFACD

SEQ ID NO: 231 - protein, 39 aa, unidentified
IEVMYPPPYLDNEKSNGTIIHVKGKHLCPSPLFPGPSKP

SEQ ID NO: 232 - protein, 12 aa, unidentified
ESKYGPPCPPCP

SEQ ID NO: 233 - protein, 5 aa, unidentified
  REPEAT 1..5: note = Any number of repeats
GGGGS

SEQ ID NO: 234 - protein, 229 aa, unidentified
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 235 - protein, 24 aa, unidentified
IYIWAPLAGTCGVLLLSLVITLYC

SEQ ID NO: 236 - protein, 27 aa, unidentified
FWVLVVVGGVLACYSLLVTVAFIIFWV

SEQ ID NO: 237 - protein, 42 aa, unidentified
KRGRKKLLYIFKQPFMRPVQTTQEEDGCSCRFPEEEEGGCEL

SEQ ID NO: 238 - protein, 41 aa, unidentified
RSKRSRLLHSDYMNMTPRRPGPTRKHYQPYAPPRDFAAYRS

SEQ ID NO: 239 - protein, 112 aa, unidentified
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 240 - protein, 112 aa, unidentified
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 241 - protein, 245 aa, unidentified
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 242 - protein, 107 aa, unidentified
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 243 - protein, 6 aa, unidentified
QDISKY

SEQ ID NO: 244 - skipped

SEQ ID NO: 245 - protein, 9 aa, unidentified
QQGNTLPYT

SEQ ID NO: 246 - protein, 18 aa, synthetic construct
GSTSGSGKPGSGEGSTKG

SEQ ID NO: 247 - protein, 120 aa, unidentified
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 248 - protein, 8 aa, unidentified
GVSLPDYG

SEQ ID NO: 249 - protein, 7 aa, unidentified
IWGSETT

SEQ ID NO: 250 - protein, 14 aa, unidentified
AKHYYYGGSYAMDY

SEQ ID NO: 251 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 252 - protein, 15 aa, synthetic construct
GGGGSGGGGSGGGGS

SEQ ID NO: 253 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 254 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 255 - DNA, 1383 bp, synthetic construct
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 256 - protein, 461 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 257 - DNA, 1467 bp, synthetic construct
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 258 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 259 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 260 - protein, 55 aa, synthetic construct
FVPVFLPAKPTTTPAPRPPTPAPTIASQPLSLRPEACRPAAGGAVHTRGLDFACD

SEQ ID NO: 261 - protein, 28 aa, synthetic construct
IYIWAPLAGTCGVLLLSLVITLYCNHRN

SEQ ID NO: 262 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 263 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 264 - protein, 10 aa, synthetic construct
RASSSVNYMD

SEQ ID NO: 265 - protein, 7 aa, synthetic construct
ATSNLAS

SEQ ID NO: 266 - protein, 13 aa, synthetic construct
SNYYGSSYWFFDV

SEQ ID NO: 267 - protein, 9 aa, synthetic construct
QQWSFNPPT

SEQ ID NO: 268 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 269 - protein, 5 aa, synthetic construct
SYNMH

SEQ ID NO: 270 - protein, 17 aa, synthetic construct
AIYPGNGDTSYNQKFKG

SEQ ID NO: 271 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 272 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 273 - protein, 15 aa, synthetic construct
RASESVSVIGAHLIH

SEQ ID NO: 274 - protein, 7 aa, synthetic construct
LASNLET

SEQ ID NO: 275 - protein, 9 aa, synthetic construct
LQSRIFPRT

SEQ ID NO: 276 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 277 - protein, 5 aa, synthetic construct
DYSIN

SEQ ID NO: 278 - protein, 17 aa, synthetic construct
WINTETREPAYAYDFRG

SEQ ID NO: 279 - protein, 8 aa, synthetic construct
DYSYAMDY

SEQ ID NO: 280 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 281 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 282 - protein, 15 aa, synthetic construct
RASESVTILGSHLIY

SEQ ID NO: 283 - protein, 7 aa, synthetic construct
LASNVQT

SEQ ID NO: 284 - protein, 9 aa, synthetic construct
LQSRTIPRT

SEQ ID NO: 285 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 286 - protein, 5 aa, synthetic construct
HYSMN

SEQ ID NO: 287 - protein, 17 aa, synthetic construct
RINTESGVPIYADDFKG

SEQ ID NO: 288 - protein, 8 aa, synthetic construct
DYLYSLDF

SEQ ID NO: 289 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 290 - protein, 8 aa, synthetic construct
GFTFSSYA

SEQ ID NO: 291 - protein, 8 aa, synthetic construct
ISGSGDYI

SEQ ID NO: 292 - protein, 14 aa, synthetic construct
AKEGTGANSSLADY

SEQ ID NO: 293 - protein, 243 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 294 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 295 - protein, 6 aa, synthetic construct
QSISSY

SEQ ID NO: 296 - skipped

SEQ ID NO: 297 - protein, 8 aa, synthetic construct
QQKYDLLT

SEQ ID NO: 298 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 299 - protein, 10 aa, synthetic construct
GGSISSSSYY

SEQ ID NO: 300 - protein, 7 aa, synthetic construct
ISYSGST

SEQ ID NO: 301 - protein, 11 aa, synthetic construct
ARDRGDTILDV

SEQ ID NO: 302 - protein, 23 aa, synthetic construct
MALPVTALLLPLALLLHAARPGS

SEQ ID NO: 303 - protein, 243 aa, synthetic construct
QVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSSGGGGSGGGGSGGGGSQIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIK

SEQ ID NO: 304 - protein, 47 aa, synthetic construct
SGTTTPAPRPPTPAPTIASQPLSLRPEACRPAAGGAVHTRGLDFACD

SEQ ID NO: 305 - protein, 243 aa, synthetic construct
QIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIKGGGGSGGGGSGGGGSQVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSS

SEQ ID NO: 306 - RNA, 4689 nt, synthetic construct
  source 1..4689: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggactacaaagaccatgacggcgactacaaggaccacgacatcgactacaaggacgacgatgataaaatggcccccaagaaaaagagaaaggtgggcattcacggcgtgcctgccgccgacaagaagtacagcatcggactggatatcggcaccaacagcgtgggctgggccgtgatcaccgacgagtacaaagtccctagcaaaaaattcaaggtgctgggcaacaccgaccggcactccatcaagaagaacctgatcggggctctgctttttgacagcggagaaaccgctgaggccacaagactgaaaagaaccgccagaaggaggtacacccgaagaaagaacagaatttgttacctgcaggagatcttttctaacgaaatggccaaggttgacgacagcttcttccacaggctggaagaatccttcctggtggaggaagataaaaagcacgagagacaccctatcttcggcaatatcgtggacgaggtcgcctaccacgagaagtaccccaccatctaccacctgagaaagaagctggtggattctacagataaggccgacctgagactcatctacctggccctggcccatatgatcaagttcagaggccacttcttaatcgaaggcgacctgaaccctgataacagcgacgtggataagctgttcatccagctggttcagacctacaaccagctatttgaggaaaaccctatcaacgcctctggcgtggacgcaaaagccatcctctccgcccggctgtccaagagcagaagactggaaaacctgattgctcagctgcctggcgaaaagaaaaacggcctgttcgggaacctgatcgctctgagcctgggcctgacccctaacttcaagagcaactttgatcttgctgaggacgccaagctgcagctgagcaaggatacctacgacgacgacctggataatctgctggcccaaatcggagatcagtacgccgatctcttcctggctgctaagaatctgagcgatgcaatcctgctgtctgacatcctgcgggtgaacaccgagatcacgaaggctccactgagcgccagtatgatcaagagatacgatgaacaccatcaggacctgaccctgctgaaggctctggtgcggcagcagctgccagaaaagtacaaggagattttcttcgaccagagcaaaaatggctatgccggctacatcgacggcggcgccagccaggaggagttctacaagttcatcaagcctatcctggagaagatggacggcaccgaggagctgctcgtgaagctgaacagagaggacctgctgcggaagcagcggaccttcgacaatggatctatcccccaccagatccacctgggcgagctgcatgccattctgagacggcaagaggatttttatcctttcctgaaagataacagagagaagatcgagaaaatcctgaccttcagaatcccttactacgtgggtcctctggcccgggggaacagccggttcgcctggatgaccagaaagagcgaggagacaataacaccttggaacttcgaagaggtggtggacaaaggggccagcgcccagagcttcatcgagcggatgacaaacttcgacaagaacctgcccaacgagaaggtgctgccaaagcacagcctgctgtatgaatacttcacagtgtacaacgaactgacaaaggtgaagtatgtgaccgagggaatgagaaagcccgcattcctgagcggcgaacaaaagaaggccatcgtggacctgctgtttaagaccaacagaaaggttaccgtgaagcagctgaaggaagattatttcaagaagatcgagtgcttcgattctgtggaaatcagcggcgtcgaagatagattcaacgccagcctgggcacctaccacgatctgctgaagatcatcaaggacaaagacttcctggacaacgaggagaacgaagacatcctggaagatatcgtgctgaccctgaccctgttcgaggaccgggaaatgatcgaagagcggctgaagacatacgcccacctgtttgatgacaaagtgatgaaacagctgaaaagacggagatacacaggctggggccgactgagcagaaagctgatcaacggaatccgggataagcagagcggcaagacaatcctggacttcctgaagtccgatggtttcgctaaccggaacttcatgcagttgatccacgacgacagcctgacctttaaggaagacattcagaaggcccaagtgtctggccagggcgacagtctgcatgagcacatcgccaacctggctggcagccctgctatcaagaagggcatcctgcagacagtgaaagtggtggacgagctggtgaaggttatgggccggcacaagccagagaatatcgtgatcgagatggcccgggagaatcagacaacccaaaagggccagaaaaactccagagaaagaatgaagagaatcgaggagggcatcaaggagctcggttctcagatcctgaaggagcaccccgtggaaaacacacagctgcagaacgagaagctgtacctgtactacctgcaaaatggcagagacatgtacgtagaccaggagctggatatcaatagactatctgattacgacgtggaccacatcgtgccccagagcttcctcaaagacgatagcatcgacaacaaggtgctgaccagaagcgacaagaaccggggcaagtctgataacgtgcccagcgaggaggtggtcaagaagatgaaaaattactggcggcaactgctgaatgctaagctgatcacccagagaaaattcgacaatctgacaaaggctgagagaggcggactgtctgagctggacaaggccggcttcatcaagagacagctggtggaaacccggcagatcaccaagcacgtggcccagatcctggacagccggatgaacaccaagtacgatgaaaacgacaagctgatcagagaggtcaaggttatcaccctgaagagcaagctagtgtccgacttcagaaaagattttcagttctacaaggtgcgggaaattaacaactaccaccacgctcacgatgcttacctgaatgcagtggtgggcacagccctgatcaagaaataccccaagctggaatctgaatttgtgtacggcgattacaaggtgtacgacgtgcgcaaaatgatcgccaagagcgaacaggaaatcggcaaagccaccgccaaatatttcttttatagcaacatcatgaattttttcaaaaccgaaatcacactggccaacggcgagattagaaagcgccccctaatcgagacaaacggagaaacaggcgagatcgtttgggacaaaggcagagacttcgccaccgtgcggaaagtgttgagcatgcctcaggtgaacatcgtgaaaaagaccgaggtgcaaaccggaggcttcagcaaggagtccatcttgcctaagagaaactccgacaagctgatcgccagaaagaaggattgggaccccaagaaatacggcggcttcgattctcctacagtggcctattctgtgctggtggtcgccaaggtggaaaagggcaagtctaaaaagttgaagtccgtgaaggaactgctgggcatcacaatcatggaacggagcagctttgaaaaaaatcccatcgacttcctggaggccaaaggatataaggaggtgaagaaggacctgatcatcaaactgcctaagtacagcctgttcgaactcgaaaacggcagaaagagaatgctggcaagcgccggcgagctgcagaaaggaaacgagctggccctaccgagcaagtatgtgaacttcctgtacctggccagccactacgagaagcttaagggcagccctgaggataatgaacagaagcaactctttgtggaacaacacaaacactacctggacgaaatcatcgagcagataagcgagttcagtaagcgtgtgatcctggctgatgccaacctggacaaggtgctgagcgcctacaacaagcatagagataagcctatcagagaacaggccgaaaacatcatccacctgttcaccctgaccaatttgggcgcccctgccgccttcaagtacttcgacaccacaatcgatagaaaacgatacaccagcacaaaagaggtgctggatgccacactgattcaccagagcatcaccggtctgtacgagacacgtatcgacctgagccagctgggaggagacaaaagacctgccgccaccaagaaggccggccaagccaagaagaagaagtgaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 307 - RNA, 4520 nt, synthetic construct
  source 1..4520: mol_type = other RNA
agcataaaagtcacatttgcttctgacatagttgtgttgactcacaaccccagaaacagacatcgaattcgctgccaccatggcccccaagaaaaagagaaaggtgggcattcacggcgtgcctgccgccgacaagaagtacagcatcggactggatatcggcaccaacagcgtgggctgggccgtgatcaccgacgagtacaaagtccctagcaaaaaattcaaggtgctgggcaacaccgaccggcactccatcaagaagaacctgatcggggctctgctttttgacagcggagaaaccgctgaggccacaagactgaaaagaaccgccagaaggaggtacacccgaagaaagaacagaatttgttacctgcaggagatcttttctaacgaaatggccaaggttgacgacagcttcttccacaggctggaagaatccttcctggtggaggaagataaaaagcacgagagacaccctatcttcggcaatatcgtggacgaggtcgcctaccacgagaagtaccccaccatctaccacctgagaaagaagctggtggattctacagataaggccgacctgagactcatctacctggccctggcccatatgatcaagttcagaggccacttcttaatcgaaggcgacctgaaccctgataacagcgacgtggataagctgttcatccagctggttcagacctacaaccagctatttgaggaaaaccctatcaacgcctctggcgtggacgcaaaagccatcctctccgcccggctgtccaagagcagaagactggaaaacctgattgctcagctgcctggcgaaaagaaaaacggcctgttcgggaacctgatcgctctgagcctgggcctgacccctaacttcaagagcaactttgatcttgctgaggacgccaagctgcagctgagcaaggatacctacgacgacgacctggataatctgctggcccaaatcggagatcagtacgccgatctcttcctggctgctaagaatctgagcgatgcaatcctgctgtctgacatcctgcgggtgaacaccgagatcacgaaggctccactgagcgccagtatgatcaagagatacgatgaacaccatcaggacctgaccctgctgaaggctctggtgcggcagcagctgccagaaaagtacaaggagattttcttcgaccagagcaaaaatggctatgccggctacatcgacggcggcgccagccaggaggagttctacaagttcatcaagcctatcctggagaagatggacggcaccgaggagctgctcgtgaagctgaacagagaggacctgctgcggaagcagcggaccttcgacaatggatctatcccccaccagatccacctgggcgagctgcatgccattctgagacggcaagaggatttttatcctttcctgaaagataacagagagaagatcgagaaaatcctgaccttcagaatcccttactacgtgggtcctctggcccgggggaacagccggttcgcctggatgaccagaaagagcgaggagacaataacaccttggaacttcgaagaggtggtggacaaaggggccagcgcccagagcttcatcgagcggatgacaaacttcgacaagaacctgcccaacgagaaggtgctgccaaagcacagcctgctgtatgaatacttcacagtgtacaacgaactgacaaaggtgaagtatgtgaccgagggaatgagaaagcccgcattcctgagcggcgaacaaaagaaggccatcgtggacctgctgtttaagaccaacagaaaggttaccgtgaagcagctgaaggaagattatttcaagaagatcgagtgcttcgattctgtggaaatcagcggcgtcgaagatagattcaacgccagcctgggcacctaccacgatctgctgaagatcatcaaggacaaagacttcctggacaacgaggagaacgaagacatcctggaagatatcgtgctgaccctgaccctgttcgaggaccgggaaatgatcgaggagcggctgaagacatacgcccacctgtttgatgacaaagtgatgaaacagctgaaaagacggagatacacaggctggggccgactgagcagaaagctgatcaacggaatccgggataagcagagcggcaagacaatcctggacttcctgaagtccgatggtttcgctaaccggaacttcatgcagttgatccacgacgacagcctgacctttaaggaagacattcagaaggcccaagtgtctggccagggcgacagtctgcatgagcacatcgccaacctggctggcagccctgctatcaagaagggcatcctgcagacagtgaaagtggtggacgagctggtgaaggttatgggccggcacaagccagagaatatcgtgatcgagatggcccgggagaatcagacaacccaaaagggccagaaaaactccagagaaagaatgaagagaatcgaggagggcatcaaggagctcggttctcagatcctgaaggagcaccccgtggaaaacacacagctgcagaacgagaagctgtacctgtactacctgcaaaatggcagagacatgtacgtagaccaggagctggatatcaatagactatctgattacgacgtggaccacatcgtgccccagagcttcctcaaagacgatagcatcgacaacaaggtgctgaccagaagcgacaagaaccggggcaagtctgataacgtgcccagcgaggaggtggtcaagaagatgaaaaattactggcggcaactgctgaatgctaagctgatcacccagagaaaattcgacaatctgacaaaggctgagagaggcggactgtctgagctggacaaggccggcttcatcaagagacagctggtggaaacccggcagatcaccaagcacgtggcccagatcctggacagccggatgaacaccaagtacgatgaaaacgacaagctgatcagagaggtcaaggttatcaccctgaaaagcaagctagtgtccgacttcagaaaagattttcagttctacaaggtgcgggaaattaacaactaccaccacgctcacgatgcttacctgaatgcagtggtgggcacagccctgatcaagaaataccccaagctggaatctgaatttgtgtacggcgattacaaggtgtacgacgtgcgcaaaatgatcgccaagagcgaacaggaaatcggcaaagccaccgccaaatatttcttttatagcaacatcatgaattttttcaaaaccgaaatcacactggccaacggcgagattagaaagcgccccctaatcgagacaaacggagaaacaggcgagatcgtttgggacaaaggcagagacttcgccaccgtgcggaaagtgttgagcatgcctcaggtgaacatcgtgaaaaagaccgaggtgcaaaccggaggcttcagcaaggagtccatcttgcctaagagaaactccgacaagctgatcgccagaaagaaggattgggaccccaagaaatacggcggcttcgattctcctacagtggcctattctgtgctggtggtcgccaaggtggaaaagggcaagtctaaaaagttgaagtccgtgaaggaactgctgggcatcacaatcatggaacggagcagctttgaaaaaaatcccatcgacttcctggaggccaaaggatataaggaggtgaagaaggacctgatcatcaaactgcctaagtacagcctgttcgaactcgaaaacggcagaaagagaatgctggcaagcgccggcgagctgcagaaaggaaacgagctggccctaccgagcaagtatgtgaacttcctgtacctggccagccactacgagaagcttaagggcagccctgaggataatgaacagaagcaactctttgtggaacaacacaaacactacctggacgaaatcatcgagcagataagcgagttcagtaagcgtgtgatcctggctgatgccaacctggacaaggtgctgagcgcctacaacaagcatagagataagcctatcagagaacaggccgaaaacatcatccacctgttcaccctgaccaatttgggcgcccctgccgccttcaagtacttcgacaccacaatcgatagaaaacgatacaccagcacaaaagaggtgctggatgccacactgattcaccagagcatcaccggtctgtacgagacacgtatcgacctgagccagctgggaggagacaaaagacctgccgccaccaagaaggccggccaagccaagaagaagaagtgatgaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 308 - RNA, 4518 nt, synthetic construct
  source 1..4518: mol_type = other RNA
agcataaaagtcacatttgcttctgacatagttgtgttgactcacaaccccagaaacagacatcgaattcgctgccaccatggcccccaagaaaaagagaaaggtgggcattcacggcgtgcctgccgccgacaagaagtacagcatcggactggatatcggcaccaacagcgtgggctgggccgtgatcaccgacgagtacaaagtccctagcaaaaaattcaaggtgctgggcaacaccgaccggcactccatcaagaagaacctgatcggggctctgctttttgacagcggagaaaccgctgaggccacaagactgaaaagaaccgccagaaggaggtacacccgaagaaagaacagaatttgttacctgcaggagatcttttctaacgaaatggccaaggttgacgacagcttcttccacaggctggaagaatccttcctggtggaggaagataaaaagcacgagagacaccctatcttcggcaatatcgtggacgaggtcgcctaccacgagaagtaccccaccatctaccacctgagaaagaagctggtggattctacagataaggccgacctgagactcatctacctggccctggcccatatgatcaagttcagaggccacttcttaatcgaaggcgacctgaaccctgataacagcgacgtggataagctgttcatccagctggttcagacctacaaccagctatttgaggaaaaccctatcaacgcctctggcgtggacgcaaaagccatcctctccgcccggctgtccaagagcagaagactggaaaacctgattgctcagctgcctggcgaaaagaaaaacggcctgttcgggaacctgatcgctctgagcctgggcctgacccctaacttcaagagcaactttgatcttgctgaggacgccaagctgcagctgagcaaggatacctacgacgacgacctggataatctgctggcccaaatcggagatcagtacgccgatctcttcctggctgctaagaatctgagcgatgcaatcctgctgtctgacatcctgcgggtgaacaccgagatcacgaaggctccactgagcgccagtatgatcaagagatacgatgaacaccatcaggacctgaccctgctgaaggctctggtgcggcagcagctgccagaaaagtacaaggagattttcttcgaccagagcaaaaatggctatgccggctacatcgacggcggcgccagccaggaggagttctacaagttcatcaagcctatcctggagaagatggacggcaccgaggagctgctcgtgaagctgaacagagaggacctgctgcggaagcagcggaccttcgacaatggatctatcccccaccagatccacctgggcgagctgcatgccattctgagacggcaagaggatttttatcctttcctgaaagataacagagagaagatcgagaaaatcctgaccttcagaatcccttactacgtgggtcctctggcccgggggaacagccggttcgcctggatgaccagaaagagcgaggagacaataacaccttggaacttcgaagaggtggtggacaaaggggccagcgcccagagcttcatcgagcggatgacaaacttcgacaagaacctgcccaacgagaaggtgctgccaaagcacagcctgctgtatgaatacttcacagtgtacaacgaactgacaaaggtgaagtatgtgaccgagggaatgagaaagcccgcattcctgagcggcgaacaaaagaaggccatcgtggacctgctgtttaagaccaacagaaaggttaccgtgaagcagctgaaggaagattatttcaagaagatcgagtgcttcgattctgtggaaatcagcggcgtcgaagatagattcaacgccagcctgggcacctaccacgatctgctgaagatcatcaaggacaaagacttcctggacaacgaggagaacgaagacatcctggaagatatcgtgctgaccctgaccctgttcgaggaccgggaaatgatcgaggagcggctgaagacatacgcccacctgtttgatgacaaagtgatgaaacagctgaaaagacggagatacacaggctggggccgactgagcagaaagctgatcaacggaatccgggataagcagagcggcaagacaatcctggacttcctgaagtccgatggtttcgctaaccggaacttcatgcagttgatccacgacgacagcctgacctttaaggaagacattcagaaggcccaagtgtctggccagggcgacagtctgcatgagcacatcgccaacctggctggcagccctgctatcaagaagggcatcctgcagacagtgaaagtggtggacgagctggtgaaggttatgggccggcacaagccagagaatatcgtgatcgagatggcccgggagaatcagacaacccaaaagggccagaaaaactccagagaaagaatgaagagaatcgaggagggcatcaaggagctcggttctcagatcctgaaggagcaccccgtggaaaacacacagctgcagaacgagaagctgtacctgtactacctgcaaaatggcagagacatgtacgtagaccaggagctggatatcaatagactatctgattacgacgtggaccacatcgtgccccagagcttcctcaaagacgatagcatcgacaacaaggtgctgaccagaagcgacaagaaccggggcaagtctgataacgtgcccagcgaggaggtggtcaagaagatgaaaaattactggcggcaactgctgaatgctaagctgatcacccagagaaaattcgacaatctgacaaaggctgagagaggcggactgtctgagctggacaaggccggcttcatcaagagacagctggtggaaacccggcagatcaccaagcacgtggcccagatcctggacagccggatgaacaccaagtacgatgaaaacgacaagctgatcagagaggtcaaggttatcaccctgaaaagcaagctagtgtccgacttcagaaaagattttcagttctacaaggtgcgggaaattaacaactaccaccacgctcacgatgcttacctgaatgcagtggtgggcacagccctgatcaagaaataccccaagctggaatctgaatttgtgtacggcgattacaaggtgtacgacgtgcgcaaaatgatcgccaagagcgaacaggaaatcggcaaagccaccgccaaatatttcttttatagcaacatcatgaattttttcaaaaccgaaatcacactggccaacggcgagattagaaagcgccccctaatcgagacaaacggagaaacaggcgagatcgtttgggacaaaggcagagacttcgccaccgtgcggaaagtgttgagcatgcctcaggtgaacatcgtgaaaaagaccgaggtgcaaaccggaggcttcagcaaggagtccatcttgcctaagagaaactccgacaagctgatcgccagaaagaaggattgggaccccaagaaatacggcggcttcgattctcctacagtggcctattctgtgctggtggtcgccaaggtggaaaagggcaagtctaaaaagttgaagtccgtgaaggaactgctgggcatcacaatcatggaacggagcagctttgaaaaaaatcccatcgacttcctggaggccaaaggatataaggaggtgaagaaggacctgatcatcaaactgcctaagtacagcctgttcgaactcgaaaacggcagaaagagaatgctggcaagcgccggcgagctgcagaaaggaaacgagctggccctaccgagcaagtatgtgaacttcctgtacctggccagccactacgagaagcttaagggcagccctgaggataatgaacagaagcaactctttgtggaacaacacaaacactacctggacgaaatcatcgagcagataagcgagttcagtaagcgtgtgatcctggctgatgccaacctggacaaggtgctgagcgcctacaacaagcatagagataagcctatcagagaacaggccgaaaacatcatccacctgttcaccctgaccaatttgggcgcccctgccgccttcaagtacttcgacaccacaatcgatagaaaacgatacaccagcacaaaagaggtgctggatgccacactgattcaccagagcatcaccggtctgtacgagacacgtatcgacctgagccagctgggaggagacaaaagacctgccgccaccaagaaggccggccaagccaagaagaagaagtgatgaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 309 - RNA, 4520 nt, synthetic construct
  source 1..4520: mol_type = other RNA
agcataaaagtcacatttgcttctgacatagttgtgttgactcacaaccccagaaacagacatcgaattcgctgccaccatggcccctaagaagaagcggaaagtcggcatccacggcgtgccagccgccgataagaagtatagcatcggcctggacatcggcaccaactctgttggatgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcagaatcccctactacgtgggccctctggccaggggaaatagcagatttgcctggatgacccggaagtccgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtataacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaacagaaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaagcacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccccaagaagtacggcggcttcgattctcctaccgtggcctactctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggcaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagctcggcggcgacaaaagacctgccgccacaaagaaagccggacaggccaaaaagaagaagtgatgaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 310 - RNA, 4518 nt, synthetic construct
  source 1..4518: mol_type = other RNA
agcataaaagtcacatttgcttctgacatagttgtgttgactcacaaccccagaaacagacatcgaattcgctgccaccatggcccctaagaagaagcggaaagtcggcatccacggcgtgccagccgccgataagaagtatagcatcggcctggacatcggcaccaactctgttggatgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcagaatcccctactacgtgggccctctggccaggggaaatagcagatttgcctggatgacccggaagtccgaggaaaccatcacaccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtataacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaacagaaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccagaacggccgggatatgtacgtggaccaagagctggacatcaaccggctgtccgactacgatgtggaccatatcgtgccccagtcttttctgaaggacgactccatcgacaacaaggtcctgaccagaagcgacaagaaccggggcaagagcgataacgtgccctccgaagaggtggtcaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaagcacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtccattctgcccaagagaaacagcgataagctgatcgcccggaagaaggactgggaccccaagaagtacggcggcttcgattctcctaccgtggcctactctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggcaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagctcggcggcgacaaaagacctgccgccacaaagaaagccggacaggccaaaaagaagaagtgatgaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 311 - RNA, 4520 nt, synthetic construct
  source 1..4520: mol_type = other RNA
agcataaaagtcacatttgcttctgacatagttgtgttgactcacaaccccagaaacagacatcgaattcgctgccaccatggcccctaagaagaaacggaaggtgggcatccacggcgtgcctgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagacagccgaggccacccggctgaagcggaccgcccggcggaggtacacccggcggaagaaccggatctgctacctgcaggagatcttcagcaacgagatggccaaggtggacgacagcttcttccaccggctggaggagagcttcctggtggaggaagacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaatctgatcgcccagctgcccggcgaaaagaagaacggcctgttcggcaacctgatcgccctgagcctgggcctgacccctaacttcaagagcaacttcgacctggccgaggacgccaagctgcagctgagcaaggacacctacgatgacgacctggacaacctgctggcccagatcggcgaccagtacgccgacctgttcctggccgccaagaacctgagcgacgccatcctgctgagcgacatcctgcgggtgaacaccgagatcaccaaggcccctctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatccctcaccagatccacctgggcgagctgcacgccatcctgaggcggcaggaggacttctaccccttcctgaaggacaaccgggagaagatcgagaagatcctgaccttcagaatcccctactacgtgggccctctggcccggggcaacagccggttcgcctggatgaccagaaagagcgaggagacaatcaccccttggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtataacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatctccggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaaacagctgaagcggaggaggtacaccggctggggccggctgagccggaagctgatcaacggcatccgggacaagcagagcggcaagaccatcctggacttcctgaagtccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggatatgtacgtggaccaggagctggacatcaaccggctgagcgactacgacgtggaccacatcgtgccccagagctttctgaaggacgacagcatcgacaacaaggtgctgacccggagcgacaagaaccggggcaagagcgacaacgtgccctctgaggaagtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacacggcagatcaccaagcacgtggcccagatcctggacagccggatgaacaccaagtacgacgagaacgacaagctgatccgggaggtgaaagtgatcaccctgaagtccaagctggtgagcgacttccggaaggacttccagttctacaaggtgcgggagatcaacaactaccaccacgcccacgacgcctacctgaacgccgtggtgggcaccgccctgatcaagaagtaccctaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcacactggccaacggcgagatccggaagcggcccctgatcgagacaaacggcgagacaggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgataagctgatcgcccggaagaaggactgggaccccaagaagtacggcggcttcgacagccccaccgtggcctacagcgtgctggtcgtggccaaagtggagaagggcaagagcaagaagctgaagtccgtgaaagagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgtccgcctacaacaagcaccgggacaagcccatccgggagcaggccgagaacatcatccacctgttcaccctgaccaacctgggcgcccctgccgccttcaagtacttcgacaccaccatcgaccggaagcggtacaccagcaccaaggaggtgctggacgccaccctgatccaccagagcatcaccggcctgtacgagacaaggatcgacctgagccagctgggcggcgacaagcggcccgccgccaccaagaaggccggccaggccaagaagaaaaagtgatgaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 312 - RNA, 4518 nt, synthetic construct
  source 1..4518: mol_type = other RNA
agcataaaagtcacatttgcttctgacatagttgtgttgactcacaaccccagaaacagacatcgaattcgctgccaccatggcccctaagaagaaacggaaggtgggcatccacggcgtgcctgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagacagccgaggccacccggctgaagcggaccgcccggcggaggtacacccggcggaagaaccggatctgctacctgcaggagatcttcagcaacgagatggccaaggtggacgacagcttcttccaccggctggaggagagcttcctggtggaggaagacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaatctgatcgcccagctgcccggcgaaaagaagaacggcctgttcggcaacctgatcgccctgagcctgggcctgacccctaacttcaagagcaacttcgacctggccgaggacgccaagctgcagctgagcaaggacacctacgatgacgacctggacaacctgctggcccagatcggcgaccagtacgccgacctgttcctggccgccaagaacctgagcgacgccatcctgctgagcgacatcctgcgggtgaacaccgagatcaccaaggcccctctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatccctcaccagatccacctgggcgagctgcacgccatcctgaggcggcaggaggacttctaccccttcctgaaggacaaccgggagaagatcgagaagatcctgaccttcagaatcccctactacgtgggccctctggcccggggcaacagccggttcgcctggatgaccagaaagagcgaggagacaatcaccccttggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtataacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatctccggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaaacagctgaagcggaggaggtacaccggctggggccggctgagccggaagctgatcaacggcatccgggacaagcagagcggcaagaccatcctggacttcctgaagtccgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggatatgtacgtggaccaggagctggacatcaaccggctgagcgactacgacgtggaccacatcgtgccccagagctttctgaaggacgacagcatcgacaacaaggtgctgacccggagcgacaagaaccggggcaagagcgacaacgtgccctctgaggaagtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacacggcagatcaccaagcacgtggcccagatcctggacagccggatgaacaccaagtacgacgagaacgacaagctgatccgggaggtgaaagtgatcaccctgaagtccaagctggtgagcgacttccggaaggacttccagttctacaaggtgcgggagatcaacaactaccaccacgcccacgacgcctacctgaacgccgtggtgggcaccgccctgatcaagaagtaccctaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcacactggccaacggcgagatccggaagcggcccctgatcgagacaaacggcgagacaggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgataagctgatcgcccggaagaaggactgggaccccaagaagtacggcggcttcgacagccccaccgtggcctacagcgtgctggtcgtggccaaagtggagaagggcaagagcaagaagctgaagtccgtgaaagagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgtccgcctacaacaagcaccgggacaagcccatccgggagcaggccgagaacatcatccacctgttcaccctgaccaacctgggcgcccctgccgccttcaagtacttcgacaccaccatcgaccggaagcggtacaccagcaccaaggaggtgctggacgccaccctgatccaccagagcatcaccggcctgtacgagacaaggatcgacctgagccagctgggcggcgacaagcggcccgccgccaccaagaaggccggccaggccaagaagaaaaagtgatgaggatccgctggagcctcggtggccatgcttcttgccccttgggcctccccccagcccctcctccccttcctgcacccgtacccccgtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 313 - RNA, 17 nt, synthetic construct
  source 1..17: mol_type = other RNA
  modified_base 1: mod_base = gm
  modified_base 2: mod_base = um
  modified_base 3: mod_base = cm
  modified_base 15: mod_base = OTHER; note = 2'-O-methyladenosine
  modified_base 16: mod_base = cm
  modified_base 17: mod_base = OTHER; note = 2'-O-methyladenosine
  modified_base 1..3: mod_base = OTHER; note = 3' phosphorothioate bonds
  modified_base 16^17: mod_base = OTHER; note = 3' phosphorothioate bond
gtctctcagctggtaca

SEQ ID NO: 314 - RNA, 20 nt, synthetic construct
  source 1..20: mol_type = other RNA
  modified_base 1: mod_base = OTHER; note = 2'-O-methyladenosine
  modified_base 2: mod_base = cm
  modified_base 3: mod_base = um
  modified_base 18: mod_base = um
  modified_base 19: mod_base = cm
  modified_base 20: mod_base = cm
  modified_base 1..3: mod_base = OTHER; note = 3' phosphorothioate bonds
  modified_base 19^20: mod_base = OTHER; note = 3' phosphorothioate bond
actcacgctggatagcctcc

SEQ ID NO: 315 - RNA, 2604 nt, synthetic construct
  source 1..2604: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggagaccgacaccctgctactctgggtcttgctgctgtgggtcccgggttcgaccggtgatatcgtgctaacccagagcccggccatcctgtcagcaagtcccggggaaaaggtcactatgacctgcagggcttcctcctctgtcaactacatggattggtaccagaagaagcccgggtcctcccctaagccgtggatctacgcgacgagcaacctggcttctggagttcccgcccgcttcagcggctctggctcgggcacatcgtactccttaaccattagccgcgtcgaggctgaggatgctgctacctattactgtcagcagtggtctttcaaccctcccaccttcggcggcggcaccaagctggagatcaagggctccacaagcggaggaggatcgggcggtggcagcggtggcggcggctcctcggaggtgcagctgcagcagtcgggcgcggagctggtgaagccaggggcctccgtgaagatgtcatgcaaggcctccggctacacgttcaccagctacaacatgcattgggtgaagcagactcctggtcagggcctggagtggattggcgccatttacccaggcaatggggacaccagctacaaccagaaatttaaaggaaaggccactttgactgctgacaagagcagttccaccgcctacatgcagctgtcctcgcttacgtctgaagattctgcggactactactgtgctcgctctaattattacggcagcagctattggttcttcgacgtgtggggcgctggtacaactgtcactgtaagctctctcgacgagagcaagtacggtcccccatgcccaccctgtcctgcacccgagttcctgggtgggccctcagtgttcctgttcccccccaagcccaaggacacactcatgatttcgcgaactcctgaggtgacctgcgtggtcgtcgatgtgagccaggaggaccctgaggtccagttcaactggtatgtggacggggtggaggtgcacaacgcgaaaacaaagcctcgggaggaacagttcaacagtacgtaccgcgtggtatcagtgctgaccgttctccaccaggactggctgaacggtaaagagtacaagtgcaaggtgtccaacaagggtctgccctcctctatagagaagaccatctccaaagccaaggggcagccacgtgaacctcaggtgtacacccttcccccgtcacaggaggagatgaccaagaaccaggtttctctcacctgcctggtgaagggcttctacccctctgacatcgccgtggagtgggagtcgaatggtcagccggagaacaactacaaaactactccgccggtgctggacagcgacggctccttcttcctgtactcccgtctgaccgtggacaagtcccgctggcaagagggcaacgtgttctcgtgttctgtaatgcatgaggccttgcacaaccactacacccagaaatccctttctctgagtctgggcaaaatgttttgggtcctggtcgtggttggcggcgtgctggcttgttactccctcttggtgaccgtggcgttcatcatcttttgggtgcgctccaagcgcagcaggggaggccactccgactacatgaacatgacgccgcgcaggcccggcccaacccgcaagcactatcagccctatgcgccgcctcgggactttgccgcgtacaggagtaagcgcggccgcaagaagctgctgtacatcttcaagcagccgtttatgagaccggtgcagacgacccaggaggaggacgggtgctcttgtcgctttcccgaggaagaggagggcgggtgcgaacttcgtgtgaagttttcacgttcagcggacgctcctgcctaccaacagggacagaatcaattatacaacgagttgaacctgggacgacgcgaggagtacgatgtattggataaacgccggggccgcgaccccgagatgggcggtaaacctcgtcgcaagaatccacaggagggcctctacaacgagctgcagaaggacaagatggccgaggcctactccgagatcggtatgaagggcgagcgccgtcgcggaaaagggcatgacgggctttatcaggggttgagcaccgccaccaaagacacttatgacgcactgcacatgcaggccctcccccctagatgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 316 - RNA, 2481 nt, synthetic construct
  source 1..2481: mol_type = other RNA
agcataaaagtcacatttgcttctgacatagttgtgttgactcacaaccccagaaacagacatcgaattcgctgccaccatggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcctggacgagagcaagtacggcccaccctgcccgccctgccccgccccagagttcctgggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgcggagcaagcggagccggggcggccacagcgactacatgaacatgaccccacggcggcccggccccacccggaagcactaccagccctacgccccaccacgggacttcgccgcctaccggagcaagaggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 317 - RNA, 2545 nt, synthetic construct
  source 1..2545: mol_type = other RNA
agcataaaagtcacatttgcttctgacatagttgtgttgactcacaaccccagaaacagacatcgaattcgctgccaccatggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcctggacgagagcaagtacggcccaccctgcccgccctgccccgccccagagttcctgggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgcggagcaagcggagccggggcggccacagcgactacatgaacatgaccccacggcggcccggccccacccggaagcactaccagccctacgccccaccacgggacttcgccgcctaccggagcaagaggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacggtgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 318 - RNA, 2481 nt, synthetic construct
  source 1..2481: mol_type = other RNA
agcataaaagtcacatttgcttctgacatagttgtgttgactcacaaccccagaaacagacatcgaattcgctgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactggacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaaaagcctctccctgtctctgggtaaaatgttttgggtgctggtggtggttggtggagtcctggcttgctatagcttgctagtaacagtggcctttattattttctgggtgaggagtaagaggagcaggggcggccacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 319 - RNA, 2545 nt, synthetic construct
  source 1..2545: mol_type = other RNA
agcataaaagtcacatttgcttctgacatagttgtgttgactcacaaccccagaaacagacatcgaattcgctgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactggacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaaaagcctctccctgtctctgggtaaaatgttttgggtgctggtggtggttggtggagtcctggcttgctatagcttgctagtaacagtggcctttattattttctgggtgaggagtaagaggagcaggggcggccacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 320 - RNA, 1911 nt, synthetic construct
  source 1..1911: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggctctgccggtcactgcattgttgctgccactcgctctgcttctgcacgcagcccgcccagacatagttatgacgcaaacaccccatagttctccagtgacgttgggccaacccgccagtatttcttgtagatcttctcaatctctggtatcacgagatggaaacacttacttgtcatggctccagcaaagacccggtcagcctcctcgactgctcatatataaaatatcaaatcgattcagcggtgtgccgaaccggttttcaggaagtggcgccgggacagatttcactcttaagatttccagggtgaaggcagaggacgttggagtttattattgtatgcaagctacacagtttccacttacttttggacaaggtacgcggctggaaatcaagggaggaggcggttcaggtggaggtggcagcggaggtggtggcagcgaggttcagcttgttcaatcaggggcagaggttaagaaacctggggaatctttgaaaatctcttgcaaagggtctggttatagctttacaagctattggattgggtgggtaagacagatgccggggaaaggactggaatggatgggtataatataccccggagattccgatactaggtatagtcctagcttccagggccaagtgacgatttcagcagacaaatccatctctacagcatatctgcagtggagttctcttaaggcctcagacaccgccatgtattactgcgcaagacaaggtgatttctggtccggttacggtggaatggatgtttggggccaagggactaccgtgacggtctcaagtattgaggttatgtatccacctccgtacctcgacaatgagaaatctaacggcacaatcatccatgtaaaagggaagcacctgtgtccctctcctttgttcccaggtccttctaagcctttctgggtgctcgtggtggttggaggtgtgctggcctgctactcacttttggttaccgtcgcatttattatattctgggtacggtctaaaagatctcggctgttgcacagtgattatatgaatatgaccccgagacggccgggcccaactaggaaacactaccaaccgtatgcccctcccagggactttgcggcctatcggtcccgcgttaagtttagccgcagtgcggacgcaccggcataccaacaaggtcaaaaccaactctacaatgaattgaacctcggcaggcgagaggaatacgatgttctcgataaaaggcggggtagagatcctgaaatggggggcaaacccagaaggaaaaatccccaagaaggtctctataatgagttgcaaaaagataagatggctgaggcgtactccgaaatcggaatgaagggtgagcggaggcgcggcaaaggccacgatggtctttatcagggcttgagtacagcaacgaaggatacgtacgatgctctccatatgcaggcacttcctcccaggtgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 321 - RNA, 1788 nt, synthetic construct
  source 1..1788: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgacatcgtgatgacccagaccccacacagcagccccgtgaccctgggccagcccgccagcatcagctgccggagcagccagagcctggtgagccgggacggcaacacctacctgagctggctgcagcagcggcccggccagccaccccggctgctgatctacaagatcagcaaccggttcagcggcgtgcccaacaggttcagcggcagcggcgccggcaccgacttcaccctgaagatcagccgggtgaaggccgaggacgtgggcgtgtactactgcatgcaggccacccagttcccactgaccttcggccagggcacccggctggagatcaagggcggaggcggcagcggaggcggcgggagcggcggcggaggcagcgaggtgcagctggtgcagagcggcgccgaggtgaagaagcccggcgagagcctgaaaatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgacttctggagcggctacggcggcatggacgtctggggccagggcacaaccgtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccacgggacttcgccgcctaccggagcagggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccacggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 322 - RNA, 1852 nt, synthetic construct
  source 1..1852: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgacatcgtgatgacccagaccccacacagcagccccgtgaccctgggccagcccgccagcatcagctgccggagcagccagagcctggtgagccgggacggcaacacctacctgagctggctgcagcagcggcccggccagccaccccggctgctgatctacaagatcagcaaccggttcagcggcgtgcccaacaggttcagcggcagcggcgccggcaccgacttcaccctgaagatcagccgggtgaaggccgaggacgtgggcgtgtactactgcatgcaggccacccagttcccactgaccttcggccagggcacccggctggagatcaagggcggaggcggcagcggaggcggcgggagcggcggcggaggcagcgaggtgcagctggtgcagagcggcgccgaggtgaagaagcccggcgagagcctgaaaatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgacttctggagcggctacggcggcatggacgtctggggccagggcacaaccgtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccacgggacttcgccgcctaccggagcagggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccacggtgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 323 - RNA, 1788 nt, synthetic construct
  source 1..1788: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggatattgtgatgacccagactccacactcctcacctgtcacccttggacagccggcctccatatcctgcaggtctagtcaaagcctcgtatccagagatggaaacacctacttgagttggcttcagcagaggccaggccagcctccaagactcctaatttataagatttctaaccggttctctggggtcccaaacagattcagtggcagtggggcagggacagatttcacactgaaaatcagcagggtgaaagctgaggatgtcggggtttattactgcatgcaagctacacagtttcccctcaccttcggccaagggacacgactggagattaaaggcggcggaggatctggcggaggtggtagtggcggaggcggatctgaggtgcagttggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagggcgatttttggagtggttatgggggtatggacgtctggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 324 - RNA, 1852 nt, synthetic construct
  source 1..1852: mol_type = other RNA
agcataaaagtcacatttgcttctgacatagttgtgttgactcacaaccccagaaacagacatcgaattcgctgccaccatggccttaccagtgaccgccttgctcctgccgctggccttgctgctccacgccgccaggccggatattgtgatgacccagactccacactcctcacctgtcacccttggacagccggcctccatatcctgcaggtctagtcaaagcctcgtatccagagatggaaacacctacttgagttggcttcagcagaggccaggccagcctccaagactcctaatttataagatttctaaccggttctctggggtcccaaacagattcagtggcagtggggcagggacagatttcacactgaaaatcagcagggtgaaagctgaggatgtcggggtttattactgcatgcaagctacacagtttcccctcaccttcggccaagggacacgactggagattaaaggcggcggaggatctggcggaggtggtagtggcggaggcggatctgaggtgcagttggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagggcgatttttggagtggttatgggggtatggacgtctggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataaaggatcccaaacaccattgtcacactccagctggagcctcggtggccatgcttcttgccccttgggcccaaacaccattgtcacactccatccccccagcccctcctccccttcctgcacccgtaccccccaaacaccattgtcacactccagtggtctttgaataaagtctgagtgggcggcatgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 325 - RNA, 1473 nt, synthetic construct
  source 1..1473: mol_type = other RNA
atggccttaccagtgaccgccttgctcctgccgctggccttgctgctccacgccgccaggccggatattgtgatgacccagactccacactcctcacctgtcacccttggacagccggcctccatatcctgcaggtctagtcaaagcctcgtatccagagatggaaacacctacttgagttggcttcagcagaggccaggccagcctccaagactcctaatttataagatttctaaccggttctctggggtcccaaacagattcagtggcagtggggcagggacagatttcacactgaaaatcagcagggtgaaagctgaggatgtcggggtttattactgcatgcaagctacacagtttcccctcaccttcggccaagggacacgactggagattaaaggcggcggaggatctggcggaggtggtagtggcggaggcggatctgaggtgcagttggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacagggcgatttttggagtggttatgggggtatggacgtctggggccaagggaccacggtcaccgtctcctcaattgaagttatgtatcctcctccttacctagacaatgaaaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 326 - RNA, 1473 nt, synthetic construct
  source 1..1473: mol_type = other RNA
atggccctgcccgtgaccgccctcctgctgcccctggccctgctcctgcacgccgcccggcccgacatcgtgatgacccagaccccacacagcagccccgtgaccctgggccagcccgccagcatcagctgccggagcagccagagcctggtgagccgggacggcaacacctacctgagctggctgcagcagcggcccggccagccaccccggctgctgatctacaagatcagcaaccggttcagcggcgtgcccaacaggttcagcggcagcggcgccggcaccgacttcaccctgaagatcagccgggtgaaggccgaggacgtgggcgtgtactactgcatgcaggccacccagttcccactgaccttcggccagggcacccggctggagatcaagggcggaggcggcagcggaggcggcgggagcggcggcggaggcagcgaggtgcagctggtgcagagcggcgccgaggtgaagaagcccggcgagagcctgaaaatcagctgcaagggcagcggctacagcttcaccagctactggatcggctgggtgcggcagatgcccggcaagggcctggagtggatgggcatcatctaccccggcgacagcgacacccggtacagccccagcttccagggccaggtgaccatcagcgccgacaagagcatcagcaccgcctacctgcagtggagcagcctgaaggccagcgacaccgccatgtactactgcgcccggcagggcgacttctggagcggctacggcggcatggacgtctggggccagggcacaaccgtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccacgggacttcgccgcctaccggagcagggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccacggtgataa

SEQ ID NO: 327 - RNA, 1473 nt, synthetic construct
  source 1..1473: mol_type = other RNA
atggctctgccggtcactgcattgttgctgccactcgctctgcttctgcacgcagcccgcccagacatagttatgacgcaaacaccccatagttctccagtgacgttgggccaacccgccagtatttcttgtagatcttctcaatctctggtatcacgagatggaaacacttacttgtcatggctccagcaaagacccggtcagcctcctcgactgctcatatataaaatatcaaatcgattcagcggtgtgccgaaccggttttcaggaagtggcgccgggacagatttcactcttaagatttccagggtgaaggcagaggacgttggagtttattattgtatgcaagctacacagtttccacttacttttggacaaggtacgcggctggaaatcaagggaggaggcggttcaggtggaggtggcagcggaggtggtggcagcgaggttcagcttgttcaatcaggggcagaggttaagaaacctggggaatctttgaaaatctcttgcaaagggtctggttatagctttacaagctattggattgggtgggtaagacagatgccggggaaaggactggaatggatgggtataatataccccggagattccgatactaggtatagtcctagcttccagggccaagtgacgatttcagcagacaaatccatctctacagcatatctgcagtggagttctcttaaggcctcagacaccgccatgtattactgcgcaagacaaggtgatttctggtccggttacggtggaatggatgtttggggccaagggactaccgtgacggtctcaagtattgaggttatgtatccacctccgtacctcgacaatgagaaatctaacggcacaatcatccatgtaaaagggaagcacctgtgtccctctcctttgttcccaggtccttctaagcctttctgggtgctcgtggtggttggaggtgtgctggcctgctactcacttttggttaccgtcgcatttattatattctgggtacggtctaaaagatctcggctgttgcacagtgattatatgaatatgaccccgagacggccgggcccaactaggaaacactaccaaccgtatgcccctcccagggactttgcggcctatcggtcccgcgttaagtttagccgcagtgcggacgcaccggcataccaacaaggtcaaaaccaactctacaatgaattgaacctcggcaggcgagaggaatacgatgttctcgataaaaggcggggtagagatcctgaaatggggggcaaacccagaaggaaaaatccccaagaaggtctctataatgagttgcaaaaagataagatggctgaggcgtactccgaaatcggaatgaagggtgagcggaggcgcggcaaaggccacgatggtctttatcagggcttgagtacagcaacgaaggatacgtacgatgctctccatatgcaggcacttcctcccaggtgataa

SEQ ID NO: 328 - RNA, 2166 nt, synthetic construct
  source 1..2166: mol_type = other RNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcactggacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaaaagcctctccctgtctctgggtaaaatgttttgggtgctggtggtggttggtggagtcctggcttgctatagcttgctagtaacagtggcctttattattttctgggtgaggagtaagaggagcaggggcggccacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 329 - RNA, 2166 nt, synthetic construct
  source 1..2166: mol_type = other RNA
atggagacagacaccctcctgctgtgggtgctgctcctgtgggtgcccggcagcaccggcgacatcgtgctgacccagagccccgccatcctgagcgccagccccggcgagaaggtgaccatgacctgccgggcctccagcagcgtgaactacatggactggtaccagaagaagcccggcagcagccccaagccctggatctacgccaccagcaacctggccagcggcgtgcccgcccggttcagcgggagcggcagcggcaccagctacagcctgaccatcagccgggtggaggccgaggacgccgccacctactactgccagcagtggagcttcaacccacccaccttcggaggcggcaccaagctggagatcaagggcagcaccagcggaggcggcagcggcggagggagcggaggcggaggcagcagcgaggtgcagctgcagcagagcggcgccgagctggtgaagcccggcgccagcgtgaagatgagctgcaaggccagcggctacaccttcaccagctacaacatgcactgggtgaagcagaccccaggccagggcctggagtggatcggcgccatctaccccggcaacggcgacacaagctacaaccagaagttcaagggcaaggccaccctgaccgccgacaagtccagcagcaccgcctacatgcagctgagcagcctgaccagcgaggacagcgccgattactactgcgcccggagcaactactacggcagcagctactggttcttcgacgtctggggcgccggcaccaccgtgaccgtgagcagcctggacgagagcaagtacggcccaccctgcccgccctgccccgccccagagttcctgggcggccccagcgtgttcctgttcccacccaagcccaaggacaccctgatgatcagccggaccccagaggtgacctgcgtcgtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtctacaccctgccacccagccaggaggagatgaccaagaaccaggtgagcctgacctgcctggtgaagggcttctaccccagcgacatcgccgtggagtgggagagcaacggccagcccgagaacaactacaagaccaccccaccagtgctggacagcgacggcagcttcttcctctacagccggctgaccgtggacaagagccggtggcaggagggcaacgtcttcagctgcagcgtgatgcacgaggccctgcacaaccactacacccagaaaagcctgagcctcagcctgggcaagatgttctgggtgctggtcgtggtgggcggcgtgctggcctgctacagcctgctggtgaccgtggccttcatcatcttctgggtgcggagcaagcggagccggggcggccacagcgactacatgaacatgaccccacggcggcccggccccacccggaagcactaccagccctacgccccaccacgggacttcgccgcctaccggagcaagaggggccggaagaagctgctctacatcttcaagcagcccttcatgcggcccgtgcagaccacccaggaggaagacggctgcagctgccggttccccgaggaggaagagggcggctgcgagctgcgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctatacaacgaactgcagaaggacaagatggccgaggcctacagcgagatcggcatgaagggcgagcggaggaggggcaagggccacgacggcctgtaccagggcctgagcaccgccaccaaggacacctacgacgccctgcacatgcaggccctgccaccacggtgataa

SEQ ID NO: 330 - RNA, 2166 nt, synthetic construct
  source 1..2166: mol_type = other RNA
atggagaccgacaccctgctactctgggtcttgctgctgtgggtcccgggttcgaccggtgatatcgtgctaacccagagcccggccatcctgtcagcaagtcccggggaaaaggtcactatgacctgcagggcttcctcctctgtcaactacatggattggtaccagaagaagcccgggtcctcccctaagccgtggatctacgcgacgagcaacctggcttctggagttcccgcccgcttcagcggctctggctcgggcacatcgtactccttaaccattagccgcgtcgaggctgaggatgctgctacctattactgtcagcagtggtctttcaaccctcccaccttcggcggcggcaccaagctggagatcaagggctccacaagcggaggaggatcgggcggtggcagcggtggcggcggctcctcggaggtgcagctgcagcagtcgggcgcggagctggtgaagccaggggcctccgtgaagatgtcatgcaaggcctccggctacacgttcaccagctacaacatgcattgggtgaagcagactcctggtcagggcctggagtggattggcgccatttacccaggcaatggggacaccagctacaaccagaaatttaaaggaaaggccactttgactgctgacaagagcagttccaccgcctacatgcagctgtcctcgcttacgtctgaagattctgcggactactactgtgctcgctctaattattacggcagcagctattggttcttcgacgtgtggggcgctggtacaactgtcactgtaagctctctcgacgagagcaagtacggtcccccatgcccaccctgtcctgcacccgagttcctgggtgggccctcagtgttcctgttcccccccaagcccaaggacacactcatgatttcgcgaactcctgaggtgacctgcgtggtcgtcgatgtgagccaggaggaccctgaggtccagttcaactggtatgtggacggggtggaggtgcacaacgcgaaaacaaagcctcgggaggaacagttcaacagtacgtaccgcgtggtatcagtgctgaccgttctccaccaggactggctgaacggtaaagagtacaagtgcaaggtgtccaacaagggtctgccctcctctatagagaagaccatctccaaagccaaggggcagccacgtgaacctcaggtgtacacccttcccccgtcacaggaggagatgaccaagaaccaggtttctctcacctgcctggtgaagggcttctacccctctgacatcgccgtggagtgggagtcgaatggtcagccggagaacaactacaaaactactccgccggtgctggacagcgacggctccttcttcctgtactcccgtctgaccgtggacaagtcccgctggcaagagggcaacgtgttctcgtgttctgtaatgcatgaggccttgcacaaccactacacccagaaatccctttctctgagtctgggcaaaatgttttgggtcctggtcgtggttggcggcgtgctggcttgttactccctcttggtgaccgtggcgttcatcatcttttgggtgcgctccaagcgcagcaggggaggccactccgactacatgaacatgacgccgcgcaggcccggcccaacccgcaagcactatcagccctatgcgccgcctcgggactttgccgcgtacaggagtaagcgcggccgcaagaagctgctgtacatcttcaagcagccgtttatgagaccggtgcagacgacccaggaggaggacgggtgctcttgtcgctttcccgaggaagaggagggcgggtgcgaacttcgtgtgaagttttcacgttcagcggacgctcctgcctaccaacagggacagaatcaattatacaacgagttgaacctgggacgacgcgaggagtacgatgtattggataaacgccggggccgcgaccccgagatgggcggtaaacctcgtcgcaagaatccacaggagggcctctacaacgagctgcagaaggacaagatggccgaggcctactccgagatcggtatgaagggcgagcgccgtcgcggaaaagggcatgacgggctttatcaggggttgagcaccgccaccaaagacacttatgacgcactgcacatgcaggccctcccccctagatgataa

SEQ ID NO: 331 - protein, 112 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIK

SEQ ID NO: 332 - protein, 16 aa, synthetic construct
RSSQSLVSRDGNTYLS

SEQ ID NO: 333 - protein, 7 aa, synthetic construct
KISNRFS

SEQ ID NO: 334 - protein, 9 aa, synthetic construct
MQATQFPLT

SEQ ID NO: 335 - protein, 127 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSSYWIG

SEQ ID NO: 336 - protein, 17 aa, synthetic construct
IIYPGDSDTRYSPSFQG

SEQ ID NO: 337 - protein, 13 aa, synthetic construct
QGDFWSGYGGMDV

SEQ ID NO: 338 - protein, 121 aa, synthetic construct
  VARIANT 33: note = X is N, Q, S, or A
  VARIANT 55: note = X is N, Q, S, or A
  VARIANT 103: note = X is N, Q, S, or A
QVQLVQSGAEVKKPGASVKVSCKASRYTFTDYXLHWVRQAPGQGLEWMGFIYPYXGGTGYAQKFQGRVTMTRDTSTSTVYMELSSLRSEDTAVYYCARDHRYXEGVSFDYWGQGTTVTVSS

SEQ ID NO: 339 - protein, 121 aa, synthetic construct; identical to SEQ ID NO: 338
  VARIANT 33: note = X is N, S, Q, or A
  VARIANT 55: note = X is N, Q, S, D, or A
  VARIANT 103: note = X is N, Q, S, or A

SEQ ID NO: 340 - protein, 111 aa, synthetic construct
  VARIANT 30: note = X is D, E, S, or A
  VARIANT 34: note = X is N, Q, S, or A
  VARIANT 57: note = X is N, Q, S, D, or A
  VARIANT 95: note = X is N, Q, S, D, or A
DIQMTQSPSSLSASVGDRVTITCRASESVXGFGXSFMNWYQQKPGKAPKLLIYLASXLESGVPSRFSGSGSGTDFTLTISSLQPEDFATYYCQQXXEXPYTFGQGTKLEIK

SEQ ID NO: 341 - protein, 120 aa, synthetic construct
  VARIANT 33: note = X is N, Q, S, or A
  VARIANT 55: note = X is N, Q, S, or A
  VARIANT 103: note = X is N, Q, S, or A
QVQLVQSGAEVKKPGASVKVSCKASRYTFTDYXLHWVRQAPGQGLEWMGFIYPYXGGTGYAQKFQGRVTMTRDTSTSTVYMELSSLRSEDTAVYYCARDHRYXEGVSFDYWGQGTLVTVS

SEQ ID NO: 342 - protein, 120 aa, synthetic construct; identical to SEQ ID NO: 341
  VARIANT 33: note = X is N, Q, S, or A
  VARIANT 55: note = X is N, Q, S, D, or A
  VARIANT 103: note = X is N, Q, S, or A

SEQ ID NO: 343 - protein, 111 aa, synthetic construct
  VARIANT 30: note = X is D, E, S, or A
  VARIANT 34: note = X is N, Q, S, or A
  VARIANT 57: note = X is N, Q, S, D, or A
  VARIANT 95: note = X is N, Q, S, D, or A
  VARIANT 96: note = X is N, Q, S, D, or A
  VARIANT 98: note = X is D, E, S, or A
EIVLTQSPGTLSLSPGERATLSCRASESVXGFGXSFMNWYQQKPGQAPRLLIYLASXLESGIPDRFSGSGSGTDFTLTISRLEPEDFAVYYCQQXXEXPYTFGQGTKVEIK

SEQ ID NO: 344 - protein, 10 aa, synthetic construct
FIYPYSGGTG

SEQ ID NO: 345 - protein, 10 aa, synthetic construct
FIYPYQGGTG

SEQ ID NO: 346 - protein, 10 aa, synthetic construct
FIYPYAGGTG

SEQ ID NO: 347 - protein, 450 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 348 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 5 aa, synthetic construct
LPXTG

SEQ ID NO: 350 - protein, 111 aa, synthetic construct
DIQLTQSPSSLSASVGDRATITCRASESVEGFGNSFMNWYQQKPGKAPKLLIYLASNLESGVPSRFSGSGSGTDFTLTISSVQPEDFATYYCQQNNEDPYTFGQGTKLEIK

SEQ ID NO: 351 - protein, 111 aa, synthetic construct
DIQLTQSPSSLSASVGDRATITCRASESVSGFGNSFMNWYQQKPGKAPKLLIYLASNLESGVPSRFSGSGSGTDFTLTISSVQPEDFATYYCQQNNEDPYTFGQGTKLEIK

SEQ ID NO: 352 - protein, 111 aa, synthetic construct
DIQLTQSPSSLSASVGDRATITCRASESVAGFGNSFMNWYQQKPGKAPKLLIYLASNLESGVPSRFSGSGSGTDFTLTISSVQPEDFATYYCQQNNEDPYTFGQGTKLEIK

SEQ ID NO: 353 - protein, 121 aa, synthetic construct
QVQLVQSGAEVKKPGASVKVSCKASRYTFTDYNLHWVRQAPGQGLEWMGFIYPYSGGTGYAQKFQGRVTMTVDYSTSTAYMELSSLRSEDTAVYYCARDHRYNEGVSFDYWGQGTTVTVSS

SEQ ID NO: 354 - protein, 121 aa, synthetic construct
QVQLVQSGAEVKKPGASVKVSCKASRYTFTDYNLHWVRQAPGQGLEWMGFIYPYQGGTGYAQKFQGRVTMTVDYSTSTAYMELSSLRSEDTAVYYCARDHRYNEGVSFDYWGQGTTVTVSS

SEQ ID NO: 355 - protein, 121 aa, synthetic construct
QVQLVQSGAEVKKPGASVKVSCKASRYTFTDYNLHWVRQAPGQGLEWMGFIYPYAGGTGYAQKFQGRVTMTVDYSTSTAYMELSSLRSEDTAVYYCARDHRYNEGVSFDYWGQGTTVTVSS

SEQ ID NO: 356 - protein, 450 aa, synthetic construct
QVQLVQSGAEVKKPGASVKISCKASRYTFTDYNLHWVRQAPGQGLEWIGFIYPYNGGTGYAQKFQGRATLTVDTSTSTAYMELSSLRSEDTAVYYCARDHRYNEGVSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 357 - protein, 450 aa, synthetic construct
QVQLVQSGAEVKKPGASVKISCKASRYTFTDYNLHWVRQAPGQGLEWIGFIYPYNGGTGYAQKFQGRATLTVDYSTSTAYMELSSLRSEDTAVYYCARDHRYNEGVSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 358 - protein, 218 aa, synthetic construct
DIQLTQSPSSLSASVGDRATITCRASESVDGFGNSFMNWYQQKPGKAPKLLIYLASNLESGVPSRFSGSGSRTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 359 - protein, 450 aa, synthetic construct
QVQLVQSGAEVKKPGASVKVSCKASRYTFTDYNLHWVRQAPGQGLEWMGFIYPYSGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 360 - protein, 450 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 361 - protein, 450 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 362 - protein, 450 aa, synthetic construct
QVQLVQSGAEVKKPGASVKVSCKASRYTFTDYNLHWVRQAPGQGLEWMGFIYPYNGGTGYAQKFQGRVTMTVDYSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 363 - protein, 119 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLG

SEQ ID NO: 364 - protein, 218 aa, synthetic construct; identical to SEQ ID NO: 348

SEQ ID NO: 365 - protein, 240 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLG

SEQ ID NO: 366 - protein, 116 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLG

SEQ ID NO: 367 - protein, 237 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLG

SEQ ID NO: 368 - protein, 115 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAP

SEQ ID NO: 369 - protein, 236 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAP

SEQ ID NO: 370 - protein, 112 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAP

SEQ ID NO: 371 - protein, 233 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAP

SEQ ID NO: 372 - protein, 111 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 373 - protein, 232 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 374 - protein, 108 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCAA

SEQ ID NO: 375 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCAA

SEQ ID NO: 376 - protein, 107 aa, synthetic construct
RTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 377 - protein, 111 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 378 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 379 - protein, 232 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 380 - protein, 108 aa, synthetic construct
ASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPCAA

SEQ ID NO: 381 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPCAA

SEQ ID NO: 382 - protein, 110 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 383 - protein, 231 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 384 - protein, 108 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 385 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 386 - protein, 108 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHT

SEQ ID NO: 387 - protein, 107 aa, synthetic construct
RTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGEC

SEQ ID NO: 388 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGEC

SEQ ID NO: 389 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHT

SEQ ID NO: 390 - protein, 106 aa, synthetic construct
ASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPC

SEQ ID NO: 391 - protein, 227 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPC

SEQ ID NO: 392 - protein, 232 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 393 - protein, 232 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 394 - protein, 218 aa, synthetic construct
DIQLTQSPSSLSASVGDRATITCRASESVSGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 395 - protein, 231 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 396 - protein, 231 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 397 - protein, 229 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 398 - protein, 229 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 399 - protein, 218 aa, synthetic construct
DIQLTQSPSSLSASVGDRATITCRASESVSGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGEC

SEQ ID NO: 400 - protein, 229 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHT

SEQ ID NO: 401 - protein, 229 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHT

(19) United States
(12) Patent Application Publication
(10) Pub. No.: US 2025/0144234 A1
(43) Pub. Date: May 8, 2025

(54) RNA for In vivo Transfection with Increased Expression

(71) Applicant: Capstan Therapeutics Inc., San Diego, CA (US)

(72) Inventors: Daiki Matsuda, San Diego, CA (US); Makan Khoshnejad, Gladwyne, PA (US); Yan Zhang, Poway, CA (US); Duy Nguyen, San Diego, CA (US); Qian-Chen Yong, San Diego, CA (US); Haig Aghajanian, Lafayette Hill, PA (US); Adrian Ion Bot, Beverly Hills, CA (US); Priya Prakash Karmali, San Diego, CA (US); Stuart Aaron Sievers, Van Nuys, CA (US)

(21) Appl. No.: 18/934,237

(22) Filed: Nov. 1, 2024

Related U.S. Application Data
(60) Provisional application No. 63/595,753, filed on Nov. 2, 2023.
(60) Provisional application No. 63/611,092, filed on Dec. 15, 2023.
(60) Provisional application No. 63/654,928, filed on May 31, 2024.
(60) Provisional application No. 63/708,529, filed on Oct. 17, 2024.

(51) Int. Cl. A61K 48/00; A61K 47/68; A61K 47/69; C07K 14/725; C07K 16/28; C07K 16/40

(52) U.S. Cl. CPC A61K 48/0033; A61K 47/6849; A61K 47/6929; C07K 14/7051; C07K 16/2803; C07K 16/2878; C07K 16/2887; C07K 16/40; C07K 2317/53; C07K 2317/622; C07K 2317/73; C07K 2319/02; C07K 2319/03

(57) ABSTRACT

This disclosure provides improved RNA molecules, including mRNA molecules that can be produced by in vitro transcription and are suitable for in vivo transfection using an appropriate delivery vehicle, such as a lipid nanoparticle (LNP) or targeted lipid nanoparticle (tLNP). The improved RNA include particular combinations of 5′ untranslated region (UTR) and 3′ UTR, particular 3′ UTRs, or particular open reading frame sequences. Also provided herein are compositions of the LNP, or tLNP with an antibody as a targeting moiety, such as anti-CD8 antibodies that are used as targeting moiety.

### CROSS-REFERENCE TO RELATED APPLICATIONS

This application claims priority U.S. provisional application No. 63/595,753, filed Nov. 2, 2023; U.S. provisional application No. 63/611,092, filed Dec. 15, 2023; U.S. provisional application No. 63/654,928, filed May 31, 2024; and U.S. provisional application No. 63/708,529, filed Oct. 17, 2024; the disclosures of which are expressly incorporated by reference herein.

### REFERENCE TO AN ELECTRONIC SEQUENCE LISTING

The instant application contains a Sequence Listing that has been submitted electronically and is hereby incorporated by reference in its entirety. The Sequence Listing was created on Oct. 28, 2024, is named “23-1871-WO_SequenceListing.xml”, and is 669,450 bytes in size.

### BACKGROUND

Reprogramming of cells has generally relied on permanent genetic modification, that is, alteration of genomic DNA. For gene therapy and cell therapy this has most often been accomplished through the use of viral vectors such as retrovirus- or adeno-associated virus-based ones whether the modification took place in vivo or ex vivo. The development of lipid nanoparticle (LNP)-delivered mRNA technology, most prominently represented by the two mRNA-based SARS-CoV-2 vaccines that have received regulatory approval and entered the market, has stimulated interest in adapting this technology to transient, in vivo reprogramming of cells for gene and cell therapy. While vaccination can be accomplished with brief low-level expression of an immunogenic protein, cell and gene therapy uses may require more robust expression. Thus, there exists a need for mRNAs achieving elevated levels and/or extended duration of expression of the encoded protein upon transfection in vivo.

### BRIEF DESCRIPTION OF DRAWINGS

FIG. 1 illustrates a schematic diagram of CAR1, CAR2, CAR7, CAR22, and CAR25 protein with signal peptide. CAR1 and CAR2 are embodiments of anti-CD19 CAR, and CAR25, CAR7, and CAR22 are embodiments of anti-CD20 CAR. CAR1 has the following domains: CD8a signal peptide (SP), anti-CD19 scFv clone 47G4 (variable light chain, VL; linker, L; variable heavy chain, VH), CD8a hinge, CD8a transmembrane domain (TM), CD28 costimulatory domain (co-stim), and CD3ζ (signaling domain (stim). CAR2 has the following domains: CD8a SP, 47G4 scFv, CD28 hinge, CD28 TM, CD28 (co-stim), and CD3ζ (stim). CAR25 has the following domains: IgG-kappa signal peptide (IgK sp), Leu16 anti-CD20 scFv (Leu16), IgG4 hinge, CD28 TM, 4-1BB co-stimulatory domain (co-stim), and CD3ζ (stim). CAR7 has the following domains: CD8a SP, 2.1.2 hybridoma anti-CD20 scFv (2.1.2), CD28 hinge, TM, and co-stimulatory domain (co-stim), and CD3ζ (stim). CAR22 has the following domains: IgK SP, Leu16 anti-CD20 scFv (Leu16), IgG4 constant domain hinge, CD28 TM, 4-11BB co-stimulatory domain (co-stim), and CD3ζ (stim).

FIG. 2A sets forth the amino acid sequences of the CAR constructs with signal peptide (SP) disclosed herein.

FIG. 2B shows the amino acid sequence of the mature CAR constructs without signal peptide (SP).

FIG. 3A illustrates activated T cells and expanded T cells.

FIG. 3B illustrates time course of CAR expression in expanded human T cells from Donor 1 (left panels) and Donor 2 (right panels) transfected with one of 20 CAR1 mRNA constructs (upper panels) or one of 46 CAR2 mRNA constructs (lower panels) formulated with CD5-targeted tLNP at mRNA concentration of 0.6 μg (see FIG. 3C for brief description of each construct). CAR expression from each construct in triplicates is shown as CAR molecules (molecules of equivalent fluorescence) per cell of total T cells (pan T cells) at time points of 6, 24, and 48 hours post transfection. Solid black lines depict the results for the mRNA constructs selected for screening described in Example 5 (see FIG. 2A and FIG. 2B for construct information). Dotted black lines depict the results for the base constructs for CAR1 (RM_61117 and RM_61362) and CAR2 (RM_61118, RM_61512 and RM_61363), and the results for the remaining constructs are depicted with solid grey lines.

FIG. 3C shows UTR pair and ORF of mRNA sequences tested in FIG. 3B.

FIG. 3D through FIG. 3F illustrates CAR1 expression in expanded human T cells from Donor 1 at 6 hours, 24 hours, and 48 hours post transfection respectively. Horizontal dotted lines in FIG. 3E indicate CAR expression levels that are two and four-fold over that from CAR1 base construct RM_61362.

FIG. 3G through FIG. 3I illustrates CAR1 expression in expanded human T cells from Donor 2 at 6 hours, 24 hours, and 48 hours post transfection respectively. Horizontal dotted lines in FIG. 3H indicate the CAR expression levels that are two and four-fold over that from CAR1 base construct RM_61362.

FIG. 3J through FIG. 3L illustrates CAR2 expression in expanded human T cells from Donor 1 at 6 hours, 24 hours, and 48 hours post transfection respectively. Horizontal dotted lines in FIG. 3K indicate the CAR expression levels that are two and four-fold over that from CAR1 base construct RM_61512.

FIG. 3M through FIG. 3O illustrates CAR2 expression in expanded human T cells from Donor 2 at 6 hours, 24 hours, and 48 hours post transfection respectively. Horizontal dotted lines in FIG. 3N indicate the CAR expression levels that are two and four-fold over that from CAR1 base construct RM_61512.

FIG. 4A illustrates an idealized CAR expression profile evaluated via three methods: 1) CAR molecules per T cell at 48 hours post transfection; 2) peak or max CAR expression (molecules per T cell); and 3) area under the curve, AUC of CAR expression (calculated by GraphPad Prism using expression at the time point, 0, 6, 24, 48 hours in the actual data presented in FIG. 4B and FIG. 4C).

FIG. 4B and FIG. 4C are scatterplot comparisons of the three evaluation methods on CAR expression following transfection via tLNPs encapsulating CAR1 mRNAs and CAR2 mRNAs, respectively. In the three boxes at upper right in the panels, Pearson correlation is shown for each pair of comparison, and displayed proportionally with grey shading in a circle, which size corresponds to −log 10 of p-value.

FIG. 4D through FIG. 4F illustrate high correlation of CAR expression from mRNA constructs in Pan T, CD4+, and CD8+ T cells. Scatterplot comparison of CAR expression in Pan T, CD4+, and CD8+ T cells from CAR1 mRNAs (top panel) and CAR2 mRNAs (bottom panel) is shown. CAR expressions from mRNA constructs via tLNP transfection of expanded T cells from human Donor 1 were evaluated via three methods: 1) CAR molecules per T cell at 48 hours post transfection (FIG. 4D); 2) peak or max CAR expression (FIG. 4E); and 3) area under the curve, AUC of CAR expression (FIG. 4F). Pearson correlation is shown for each pair of comparison, and displayed proportionally with grey shading in a circle, which size corresponds to −log 10 of p-value.

FIG. 5A illustrates a time course of CAR1 (top) and CAR2 (bottom) expression in freshly activated human T cells of Donor 3 (left panels) and Donor 4 (right panels). Freshly activated T cells were transfected with 14 CAR1 mRNA constructs (upper panels) or 24 CAR2 mRNA constructs (lower panels) formulated with CD5-targeted tLNP. CAR expression from each construct in triplicates is shown as CAR molecules (molecules of equivalent soluble fluorochrome—MESF) per cell of total T cells (pan T cells) at time points of 24, 48, and 72 hours post transfection. Results for the mRNA constructs selected for screening described in Example 5 are depicted with solid black lines. The results for the base constructs for CAR1 (RM_61117 and RM_61362) and CAR2 (RM_61118 and RM_61512), are depicted with dotted black lines and for the remaining constructs with solid grey lines. Transfection of each test article was performed at least in triplicate.

FIG. 5B through FIG. 5D illustrate CAR1 expression from the 14 constructs in activated human T cells from Donor 3 at 24 hours, 48 hours, and 72 hours post transfection respectively. Horizontal dotted lines in FIG. 5B indicate the CAR expression levels that are two and three-fold over that from CAR1 base construct RM_61362 (see FIG. 3C for brief description of each construct).

FIG. 5E through FIG. 5G illustrate CAR1 expression from the 14 constructs in activated human T cell from Donor 4 at 24 hours, 48 hours, and 72 hours post transfection respectively. Horizontal dotted lines in FIG. 5E indicate the CAR expression levels that are two and three-fold over that from CAR1 base construct RM_61362 (see FIG. 3C for brief description of each construct).

FIG. 5H through FIG. 5J illustrate CAR2 expression from the 24 constructs in activated human T cell from Donor 3 at 24 hours, 48 hours, and 72 hours post transfection respectively. Horizontal dotted lines in FIG. 5H indicate the CAR expression levels that are two and four-fold over that from CAR2 base construct RM_61512 (see FIG. 3C for brief description of each construct).

FIG. 5K through FIG. 5M illustrate CAR2 expression from the 24 constructs in activated human T cell from Donor 4 at 24 hours, 48 hours, and 72 hours post transfection respectively. Horizontal dotted lines in FIG. 5K indicate the CAR expression levels that are two and four-fold over that from CAR2 base construct RM_61512 (see FIG. 3C for brief description of each construct).

FIG. 6 illustrates in vitro CAR function (Nalm6 killing activity) and CAR expression from mRNA constructs on freshly activated human T cells. Freshly activated T cells from Donor 4 were transfected with CAR1 and CAR2 mRNA formulated with CD5-targeting tLNP (see FIG. 3C for brief description of each construct). The graph shows the CAR expression in CAR molecules (molecules of equivalent soluble fluorochrome—MESF per cell on total T cells from CAR1 mRNA (triangles) and CAR2 mRNA (circles) at 48 hours post transfection and GFP signals from Nalm 6 cells after 48 hours of post co-culturing. The data points from base constructs are shown as open triangles for CAR1 base constructs RM_61117 and RM_61362, or filled circles for CAR2 base constructs RM_61118 and RM_61512. Those from the improved constructs are shown as filled triangles for CAR1 mRNA and open circles for CAR2 mRNA. Data point of untransfected cells is shown as an asterisk.

FIG. 7 illustrates comparison of the effect of codon optimization methods on CAR expression of CAR1 and CAR2 mRNA having a UTR pair of UTR_1-UTR_11 (TEV 5′ UTR and XBG 3′ UTR). Up to seven different methods of codon optimization were applied to CAR1 and CAR2 open reading frame (ORF) to provide optimized CAR mRNA constructs. Each data point of CAR1 (open circle) and CAR2 (filled circle) represents the CAR expression in AUC of CAR expression on total T cells with Doners 1 to 4. Transfection of each test article was performed at least in triplicate.

FIG. 8A illustrates comparisons of UTRs and codon optimization methods on CAR expression of CAR1 and CAR2 mRNA in expanded T cells of Donor 1 and 2. The effect of up to fifteen 5′-3′ UTR pairs on CAR expression were compared for each of the seven different ORF coding sequences of CAR1 (open circle) and CAR2 CAR (filled circle). CAR expression levels were shown as average rank order from normalized rank sum calculated from three different CAR expression criteria: CAR molecules per cell of total T cells, peak expression of CAR molecules per cell of total T cells and AUC of CAR expression on total T cells. The line graph that connects the average rank scores of UTR pairs was shown to improve the visibility and comparison of the UTR effect within the same codon optimization method. Transfection of each test article was performed at least in triplicate.

FIG. 8B shows 5′ UTR sequences used for mRNA screening and their core sequences.

FIG. 8C shows 3′ UTR sequences used for mRNA screening and their core sequences.

FIG. 9A and FIG. 9B show CAR1 and CAR2 expression as mean rank sum scores, respectively, of screening in Examples 2 and 3 shown in FIG. 5A through FIG. 8A.

FIG. 10 is construct/test article list with data plotted in FIG. 6. These constructs emerged from screening in Examples 2 and 3 shown in FIG. 5A through FIG. 8A.

FIG. 11A through FIG. 11C illustrate CAR expression from 3 CAR1 & 11 CAR2 improved mRNAs via transfection of human expanded, freshly activated, and rested T cells, respectively, with CD8-targeted tLNP, respectively, at 24 hours post transfection.

FIG. 12 illustrates CAR expression on T cells in blood (Day 8) in NCG-PBL (Donor 7) mice—24 hours post 3rd treatment.

FIG. 13 illustrates Nalm6-tumor burden monitoring in NCG-PBL (Donor 7) mice after treatment with improved CAR2 mRNA formulated in CD8-targeted LNP (screening described in Example 5). The Y axis shows luminescence from luciferin-luciferase reaction in photons per second. Each line represents data from an individual mouse.

FIG. 14 illustrates CAR expression on T cells in blood in NHP—0, 8, 24 hours post dose of CD5-targeted tLNP with base (RM_61512) or improved (RM_61461) CAR2 mRNA or PBS.

FIG. 15 illustrates CAR25 (anti-CD20 CAR) expression level in expanded human T cells of Donor 7 and Donor 5 transfected with CD5-targeted LNP comprising CAR25 mRNA (CD20 CAR).

FIG. 16A through FIG. 16C present flow cytometry data for human PBMC from two donors (A and B) transfected in vitro with CD8-targeted tLNPs encapsulating improved mRNAs encoding an anti-CD19 CAR2 (tLNP-98219) or an anti-CD20 CAR (tLNP-982520), or mCherry mRNA, at doses of 0.2, 0.6, or 2 μg mRNA. FIG. 16A depicts CAR expression level (molecules per cell based on molecular equivalents of soluble fluorochrome (MESF)) in CD8⁺ cells 24 hours post transfection. FIG. 16B depicts expression level (as median fluorescence intensity) of the activation marker CD69 in both CD8⁺ and CD4⁺ cells 24 hours post transfection. FIG. 16C depicts the number of B cells (normalized to counting beads) 72 hours post transfection.

FIG. 17A through FIG. 17C present flow cytometry data for spleen cells from NOD scid gamma (NSG) immunodeficient mice engrafted with human PBMCs and transfected in vivo with CD8-targeted tLNPs encapsulating improved mRNAs encoding an anti-CD19 CAR2 (tLNP-98219) or mCherry mRNA, at doses of 10 or 30 μg mRNA per animal. FIG. 17A depicts the percentage of CD3⁺, CD4⁺, or CD8⁺ splenic T cells that express the CAR. FIG. 17B depicts the level of expression in the engineered CD3+ or CD8⁺ splenic T cells as the number of CAR molecules per cells (based on MESF). FIG. 17C depicts the percentage of B cells in human CD45⁺ spleen cells in the mice transfected to express mCherry or CAR2 in their CD8⁺ T cells.

FIG. 18A through FIG. 18D present flow cytometry data from NHP administered CD2-, CD5-, or CD8-targeted tLNP encapsulating mRNA encoding either CAR2 (an anti-CD19 CAR) or CAR25 (an anti-CD20 CAR). FIG. 18A depicts transfection efficiency as the percentage of CAR⁺ cells of various cell types (CD8⁺ T cells, CD8⁻CD4⁺ T cells) expressing the relevant CAR for blood samples at various time points in the study and for necropsy tissue samples. FIG. 18B depicts the amount of B cells present in blood samples at various time points in the study as the number of cells per μL (upper panels) and as the percentage of lymphocytes (lower panels) for the animals that received tLNPs encapsulating mRNA encoding the anti-CD20 CAR for tLNP targeted to either CD8, CD2, or CD5. FIG. 18C and FIG. 18D depicts the proportion and number, respectively, of B cells present in tissue samples at study termination as the percentage of lymphocytes in spleen, liver, lymph node, and bone marrow (FIG. 18C) and as the number of B cells per gram of tissue in spleen and liver (FIG. 18D) for the animals that received tLNPs encapsulating mRNA encoding the anti-CD20 CAR for tLNP targeted to either CD8, CD2, or CD5.

FIG. 19A shows the effectiveness of tLNP-encapsulating mRNA constructs encoding spCAS9 in knocking out expression of CD3 in T cell receptor (TCR) α chain and β2-microglobulin in T cells. In the upper two panels the data has been normalized to the base construct RM_61101 to show the fold change in % knock-out. The lower panel reports the structural elements present in each of the tested mRNAs.

FIG. 19B shows the effectiveness of mRNA constructs encoding spCAS9 in knocking out expression of β2-microglobulin in CD34+ hematopoietic stem and progenitor cells (HPSCs) from Plexifor-mobilized peripheral blood (mPB). The data has been normalized to the base construct RM_61101 to show the fold change in % knock-out.

FIG. 20 shows expression of CAR, as CAR molecules per cell based on MESF, on CD8+ T cells transfected with CD8-targeted tLNP encapsulating mRNA constructs encoding anti-CD20 CAR7 or CAR22, in the upper panel. The lower panel reports the structural elements present in each of the tested mRNAs.

FIGS. 21A-21D depicts various measures of activity of human PBMC transfected in vitro with CD8-targeted tLNP encapsulating mRNA constructs encoding anti-CD20 CAR7 or CAR22. CD8+ T cells were transfected and expressing anti-CD20 CAR payload and were cytotoxic against B cells present in the PBMC. Sufficient tLNP to provide 0.2, 0.6, or 2 μg of mRNA was used in the individual transfection reactions. FIG. 21A shows killing of B cells in the culture. FIG. 21B shows increase in T cell activation by measurement of CD69 expression level in the transfected PBMC. Flow cytometry was conducted 24 hours post-transfection. FIG. 21C shows increase in T cell activation by measurement of CD25 expression level in the transfected PBMC. Flow cytometry was conducted at 24 hours post-transfection. FIG. 21D shows increased cytokine TNFα release from transfected T cells in the PBMC. Supernatants were collected at 48 hours post-transfection and assayed for TNFα. Each bar represents a single data point.

FIG. 22A and FIG. 22B show percentages and numbers of B cells in the spleen of NSG immunodeficient mice engrafted with human PBMCs at the indicated time after administration of tLNPs. FIG. 22A shows B cells in spleen as a percentage of human immune cells (CD45+) in mice treated with CD8-targeted tLNP encapsulating either mCherry mRNA or an anti-CD19 CAR mRNA (RM_61461). FIG. 22B shows total B cell counts per microliter of splenic single cell suspension (calculated using counting beads in the same samples). Each dot represents one animal, bar height is mean, and wickers indicate standard deviation. Samples with low viability (less than 5% live cells in the single suspension) were excluded from the analysis.

FIG. 23A through FIG. 23E show mCherry and CAR expression in CD8+ T cells in NSG immunodeficient mice engrafted with human PBMCs at the indicated time after administration of tLNPs. FIG. 23A shows frequency of mCherry+ CD8+ and CD4+ T cells in mice treated with CD8-targeted tLNP encapsulating mCherry mRNA. FIG. 23B shows mCherry median fluorescence intensity (MFI) in the mCherry+ CD8+ T cells (n=5 per group). FIG. 23C shows frequency of CAR expression in CD8+ and CD4+ T cells in mice treated with CD8-targeted tLNP encapsulating anti-CD19 CAR mRNA (RM_61461). FIG. 23D shows CAR median fluorescence intensity (MFI) in the CAR+ CD8+ T cells (n=10 per group). FIG. 23E shows quantification of CAR molecules per CAR+ CD8+ T cell based on MESF (n=10/group). All results were obtained from spleen samples. Each dot represents one animal, bar height is mean, and wickers indicate standard deviation. Mice with less than 5% live human CD45+ cells in the spleen were excluded from this analysis.

FIG. 24A and FIG. 24B show B cell frequency and CAR expression after treatment of NCG immunodeficient mice engrafted with human PBMCs with CD8-targeted tLNP encapsulating anti-CD19 CAR mRNA (RM_61461) compared to controls. FIG. 24A shows percentage of B cells in spleen (upper panel) and blood (lower panel) after treatment with CD8-targeted tLNP encapsulating mCherry mRNA (7.5 μg), anti-CD19 CAR mRNA (RM_61461) (indicated doses), or PBS control. Each dot represents one animal. Frequency of B cells was measured by the % of B cells in human CD45+ cells. FIG. 24B shows CAR expression in the blood and spleen in the different dosage and treatment groups. N=5 mice in the mCherry mRNA-treated group and 10 mice in each of the anti-CD19 CAR mRNA (RM_61461)- and PBS-treated groups.

FIG. 25A and FIG. 25B show evaluation of B cell depletion and recovery time course across treatments (with CD8-targeted tLNPs encapsulating mRNA encoding anti-CD19 CAR, human anti-CD20 CAR, or mCherry), dose regimens, and time points. The 0 days post treatment data are from pre-treatment samples. FIG. 25A shows longitudinal measure of B cell frequency within human CD45+ cells in the blood of NSG mice engrafted with human CD34+ cells. Each line represents an individual animal in the various dose regimens (daily, every 2 days, and every 3 days). FIG. 25B shows frequency of B cells at the terminal time point (Day 21 post final administration) for each dose regimen.

FIG. 26 shows B cell composition based on CD19 and CD20 expression. Each bar graph for the blood, spleen, and bone marrow presents the composition of the human CD45+CD3− fraction on each tissue, composed by four subsets based on CD19 and CD20 expression: CD19+CD20− B cells, CD19+CD20+ B cells, CD19-CD20+ B cells, and non-B cells (CD19−CD20−).

FIG. 27A and FIG. 27B show the frequency of B cell subsets for each dose regimen in spleen (FIG. 27A) and bone marrow (FIG. 27B) 21 days after treatment. The percentage of each subset was calculated based on a parent population of CD19⁺ B cells.

FIG. 28 shows bioluminescence images of NSG mice at the indicated time point. The NCG mice were engrafted with 1×10⁶ human PBMCs on day −18 and inoculated with 5×10⁵ Nalm6-luciferase tumor cells on day −8. Mice were bled and staged on day −1 based on tumor burden and human CD45+ cell engraftment.

### DETAILED DESCRIPTION

This disclosure provides mRNA molecules having structures conferring an increased level and/or duration of expression of the encoded polypeptide by inclusion of particular UTRs and UTR combinations and/or modification of the coding sequence for the encoded polypeptide, as well as other nucleic acid molecules comprising similar combinations of sequence elements. These mRNAs are particularly suited for in vivo transfection when delivered as a payload encapsulated in LNPs or tLNPs and this disclosure further provides formulations and pharmaceutical compositions comprising such LNPs or tLNPs, as well as methods of making and using them.

While this disclosure is capable of being embodied in various forms, the descriptions below of several embodiments are made with the understanding that this disclosure is to be considered as an exemplification of the invention and is not intended to limit the invention to the specific embodiments illustrated.

Headings are provided for convenience only and are not to be construed to limit the disclosure or claimed subject matter in any manner. Embodiments illustrated under any heading can be combined with embodiments illustrated under any other heading.

To the extent any materials incorporated herein by reference conflict with this disclosure, this disclosure controls.

Prior to setting forth this disclosure in more detail, it can be helpful to provide abbreviations and definitions of certain terms to be used herein. Additional definitions are set forth throughout this disclosure.

### (1) Definitions

As used in the specification and claims, the singular form “a,” “an,” and “the” includes plural references unless the context clearly dictates otherwise. It should be understood that the terms “a” and “an” as used herein refer to “one or more” of the enumerated components.

The use of the alternative (e.g., “or”) should be understood to mean either one, both, or any combination thereof of the alternatives.

The term “about” as used herein in the context of a number refers to a range centered on that number and spanning 10% less than that number and 10% more than that number. The term “about” used in the context of a range refers to an extended range spanning 10% less than that the lowest number listed in the range and 10% more than the greatest number listed in the range.

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

Unless the context requires otherwise, throughout this specification and the accompanying claims, the word “comprise” and variations thereof, such as, “comprises” and “comprising” are to be construed in an open, inclusive sense, that is, as “including, but not limited to.” As used herein, the terms “include” and “comprise” are used synonymously.

The phrase “at least one of” when followed by a list of items or elements refers to an open-ended set of one or more of the elements in the list, which can, but does not necessarily, include more than one of the elements.

“Derivative,” as used herein, refers to a chemically or biologically modified version of a compound that is structurally similar to a parent compound and (actually or theoretically) derivable from that parent compound. Generally, a “derivative” differs from an “analogue” in that a parent compound can be the starting material to generate a “derivative,” whereas the parent compound is not necessarily be used as the starting material to generate an “analogue.” A derivative may have different chemical or physical properties than the parent compound. For example, a derivative may be more hydrophilic or hydrophobic, or it may have altered reactivity as compared to the parent compound. Although a derivative can be obtained by physical (for example, biological or chemical) modification of the parent compound, a derivative can also be conceptually derived, for example, as when a protein sequence is designed based on one or more known sequences, an encoding nucleic acid is constructed, and the derived protein obtained by expression of the encoding nucleic acid.

As used herein, “lipid nanoparticle” (LNP) means a solid particle, as distinct from a liposome having an aqueous lumen. The core of an LNP, like the lumen of a liposome, is surrounded by a layer of lipid that can be, but is not necessarily, a continuous lipid monolayer, a bilayer, or multi-layer having three or more lipid layers.

“Artificial sequence,” or “synthetic sequence” as used herein, refers to an amino acid or nucleotide sequence that is devised to serve a specific purpose and that is not derived from a particular sequence existing in nature. The purpose of such sequences can include linkers, spacers, restrictions sites, and untranslated regions, among others.

As used herein “transfection” or “transfecting” refers to the introduction of nucleic acids into cells by non-viral methods. Transfection can be mediated by calcium phosphate, cationic polymers, magnetic beads, electroporation and lipid-based reagents. In particular embodiments disclosed herein transfection is mediated by solid lipid nanoparticles (LNP) including targeted LNP (tLNP). The term transfection is used in distinction to transduction—transfer of genetic material from cell to cell or virus to cell—and transformation—the uptake of extracellular genetic material by the natural processes of a cell. As used herein, phrases such as “delivering a nucleic acid into a cell” are synonymous with transfection.

“Reprogramming,” as used herein with respect to immune cells, refers to changing the functionality of an immune cell with respect to antigenic specificity by causing expression of an exogenous T cell receptor (TCR), a chimeric antigen receptor (CAR), or an immune cell engager (collectively termed “reprogramming agents”). Generally, T lymphocytes and natural killer (NK) cells can be reprogrammed with a TCR, a CAR, or an immune cell engager while only a CAR or an immune cell engager is used in reprogramming monocytes. In the case of an immune cell engager, the immune cells engaged and redirected against the target cell antigen of the immune cell engager are reprogrammed cells whether or not they express the reprogramming agent. Reprogramming can be transient or durable depending on the nature of the engineering agent.

“Engineering agent,” as used herein, refers to agents that confer the expression of a reprogramming agent by an immune cell, particularly a non-B lymphocyte or monocyte. Engineering agents can include nucleic acids, including mRNA that encode the reprogramming agent. Engineering agents can also include nucleic acids that are or encode components of gene editing systems such as RNA-guided nucleases, guide RNA, and nucleic acid templates for knocking-in a reprogramming agent or knocking-out an endogenous antigen receptor. Gene editing systems comprise base-editors, prime-editors or gene-writers. RNA-guided nucleases include CRISPR nucleases such as Cas9, Cas12, Cas13, Cas3, CasMINI, Cas7-11, and CasX. For transient expression of a reprogramming agent, such as a CAR, an mRNA encoding the reprogramming agent can be used as the engineering agent. For durable expression of the reprogramming agent, such as an exogenous, modified, or corrected gene (and its gene product), the engineering agent can comprise mRNA-encoded RNA-directed nucleases, guide RNAs, nucleic acid templates and other components of gene/genome editing systems.

Examples of gene editing components that are encoded by a nucleic acid molecule include an mRNA encoding an RNA-guided nuclease, a gene or base editing protein, a prime editing protein, a Gene Writer protein (e.g., a modified or modularized non-long terminal repeat (LTR) retrotransposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-MLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Other components include a guide RNA (gRNA), a single guide RNA (sgRNA), a prime editing guide RNA (pegRNA), a clustered regularly interspaced short palindromic repeat (CRISPR) RNA (crRNA), a trans-activating clustered regularly interspaced short palindromic repeat (CRISPR) RNA (tracrRNA), or a DNA molecule to be inserted or serve as a template for double-strand break (DSB) repair at a specific genomic locus. Genome-, gene-, and base-editing technology are reviewed in Anzalone et al., Nature Biotechnology 38:824-844, 2020, Sakuma, Gene and Genome Editing 3-4:100017, 2022, and Zhou et al., MedComm 3(3):e155, 2022, each of which is incorporated by reference for all that they teach about the components and uses of this technology to the extent that it does not conflict with the present disclosure.

“Conditioning agent,” as used herein, refers to a biological response modifier (BRM) that enhances the efficiency of engineering an immune cell, expands the number of immune cells available to be engineered or the number of engineered cells in a target tissue (for example, a tumor, fibrotic tissue, or tissue undergoing autoimmune attack), promotes activity of the engineered cell in a target tissue, or broadens the range of operative mechanisms contributing to a therapeutic immune reaction. A conditioning agent may be provided by delivering an encoding nucleic acid in a tLNP. Exemplary BRMs include cytokines, such as IL-7, IL-15, or IL-18.

The term “immune cell,” as used herein, can refer to any cell of the immune system. However, particular aspects can exclude polymorphonuclear leukocytes and/or B cells, or be limited to non-B lymphocytes such as T cell and/or NK cells, or to monocytes such as dendritic cells and/or macrophages in their various forms.

The term “nucleic acid” or “nucleic acid molecule,” as used herein, refers to either an RNA or DNA molecule, especially those encoding an expressible polypeptide, where context does not dictate otherwise. Description of the disclosed embodiments focuses primarily on improved mRNA molecules having the structure of a canonical mRNA. However, polypeptides can also be encoded in and expressed from circular and self-amplifying (also known as self-replicating) RNA molecules. Accordingly, the sequence of any of the herein disclosed linear mRNA molecules can be incorporated into a circular or self-amplifying/self-replicating RNA molecule. Similarly, each of these RNA molecules can be encoded as a DNA molecule. Each of the disclosed nucleic acid sequences, RNA or DNA, should be understood to disclose the corresponding DNA or RNA sequence, respectively.

The term “base construct”, as used herein, refers to mRNA constructs comprising a 5′ UTR, an ORF containing a codon optimized sequence encoding a protein, and a 3′ UTR, as could commonly be constructed for in vivo transfection (see, for example, Karikó et al., Mol. Ther, 20(5):948-953, 2012). Furthermore, expression of the mRNA base constructs is used as base level standard to which expression of other improved mRNA constructs are compared.

As used herein, “antibody” refers to a protein comprising an immunoglobulin domain having hypervariable regions determining the specificity with which the antibody binds antigen, termed complementarity determining regions (CDRs). The term antibody can thus refer to whole antibodies (also referred to as intact or full-length antibodies) as well as antibody fragments and constructs comprising an antigen binding portion of a whole antibody. While the canonical natural antibody has a pair of heavy and light chains, camelids (from camels, alpacas, llamas, and the like) produce antibodies with both the canonical structure and antibodies comprising only heavy chains. The variable region of the camelid heavy chain-only antibody has a distinct structure with a lengthened CDR3 referred to as VHH or, when produced as a fragment, a nanobody. Antigen binding fragments and constructs of antibodies include F(ab)₂, F(ab′), F(ab′)₂, F(ab), minibodies, Fv, single-chain Fv (scFv), diabodies, and VH. Such elements can be combined to produce bi- and multi-specific reagents, such as BiTEs (bispecific T-cell engagers). The term “monoclonal antibody” arose out of hybridoma technology but is now used to refer to any singular molecular species of antibody regardless of how it was originated or produced. Antibodies can be obtained through immunization, selection from a naïve or immunized library (for example, by phage display), alteration of an isolated antibody-encoding sequence, or any combination thereof. Numerous antibodies that can be used as binding moieties are known in the art. An excellent source of information about antibodies for an International Non-proprietary Name (INN) has been proposed or recommended, including sequence information, is Wilkinson & Hale, 2022, MAbs 14(1):2123299, including its Supplementary Tables, which is incorporated by reference herein for all that it teaches about individual antibodies and the various antibody formats that can be constructed. U.S. Pat. No. 11,326,182 and especially its Table 9 entitled “Cancer, Inflammation and Immune System Antibodies,” is a source of sequence and other information for a wide range of antibodies including many that do not have an INN and is incorporated herein by reference for all that it teaches about individual antibodies.

An antibody or other binding moiety (or a fusion protein thereof) “specifically binds” to a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains (or fusion proteins thereof) can be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 107 M⁻¹, up to 10⁶ M⁻¹, up to 10⁵ M⁻¹. Alternatively, affinity can be defined as an equilibrium dissociation constant (K_{D}) of a particular binding interaction with units of Molarity (M) (e.g., 10⁻⁵ M to 10⁻¹³ M). Affinities of binding domain polypeptides and fusion proteins according to this disclosure can be readily determined using conventional techniques (see, e.g., Scatchard et al., 1949, Ann. N.Y. Acad. Sci. 51:660; and U.S. Pat. Nos. 5,283,173; 5,468,614, or the equivalent).

As used herein, a “binder”, “binding moiety” or “targeting moiety” refers to a protein, polypeptide, oligopeptide or peptide, carbohydrate, nucleic acid, or combinations thereof capable of specifically binding to a target or multiple targets. “Targeting moiety” is frequently used herein to refer to a binding moiety for a targeted lipid nanoparticle that can mediate specific binding to a cell surface protein to promote preferential interaction of the targeted lipid nanoparticle with cells expressing that protein. A binding domain includes any naturally occurring, synthetic, semi-synthetic, or recombinantly produced binding partner for a biological molecule or another target of interest. Exemplary binding moieties of this disclosure include an antibody or antigen binding domain thereof, a Fab′, F(ab′)₂, Fab, Fv, rlgG, scFv, hcAb (heavy chain antibody), a single domain antibody, VHH, VNAR, sdAb, nanobody, receptor ectodomain or ligand-binding portions thereof, or ligand (e.g., cytokines, chemokines, saccharides, or glycoconjugates). A “Fab” region (fragment antigen-binding region) is the part of an antibody that binds to antigens and includes the variable region and first heavy chain domain (CH1) of the heavy chain linked to the light chain via an inter-chain disulfide bond. In other embodiments, a binding moiety comprises a ligand-binding domain of a receptor or a receptor ligand. In some embodiments, a binding moiety can have more than one specificity including, for example, bispecific or multispecific binders. A variety of assays are known for identifying binding moieties of this disclosure that specifically bind a particular target, including Western blot, ELISA, biolayer interferometry, and surface plasmon resonance. A binding moiety, such as a binding moiety comprising immunoglobulin light and heavy chain variable domains (e.g., scFv), can be incorporated into a variety of protein scaffolds or structures as described herein, such as an antibody or an antigen binding fragment thereof, a scFv-Fc fusion protein, or a fusion protein comprising two or more of such immunoglobulin binding domains.

As used throughout this disclosure, “identical” or “identity” refer to the similarity between a DNA, RNA, nucleotide, amino acid, or protein sequence to another DNA, RNA, nucleotide, amino acid, or protein sequence, respectively. Identity can be expressed in terms of a percentage of sequence identity of a first sequence to a second sequence. Percent (%) sequence identity with respect to a reference RNA sequence can be the percentage of RNA nucleotides in a candidate sequence that are identical with the RNA nucleotides in the reference RNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity. As used throughout this disclosure, the percent sequence identity values is generated using the NCBI BLAST 2.0 software as defined by Altschul et al., “Gapped BLAST and PSI-BLAST: a new generation of protein database search programs,” Nucleic Acids Res. 2007, 25, 3389-3402, with the parameters set to default values.

### (II) RNA

Certain aspects of this disclosure relate to improved RNA molecules, such as mRNA molecules that can be produced by in vitro transcription and are suitable for in vivo transfection using an appropriate delivery vehicle, such as a lipid nanoparticle (LNP) or targeted lipid nanoparticle (tLNP) as described in further detail herein. In various aspects, RNA molecules of this disclosure comprise particular 5′ untranslated regions (UTRs), particular 3′ UTRs, a particular open reading frame sequence, or any combinations thereof. Combinations of these RNA components have been selected to achieve greater protein expression levels in vitro and in vivo.

A eukaryotic mRNA typically has the following structural elements in 5′ to 3′ order: a 5′ cap, a 5′ UTR, an open reading frame (ORF) beginning with an initiator codon, one or more termination codons, a 3′ UTR, and a poly(A) tail. Efficiently translatable ORFs typically comprise at least about 80 codons (Aspden et al., 2014, eLife 3: e03528).

In certain aspects, this disclosure provides a method of tLNP-based screening for mRNA encoding a protein, for example, a CAR, with an improved level of expression, comprising:
a. formulating tLNP compositions comprising mRNA payloads having various combinations of UTRs and/or ORFs;
b. transfecting mammalian cells with the tLNP compositions; and
c. determining mRNAs having an elevated level of expression compared to the wild-type or parent RNA molecule.

mRNA according to the herein disclosed embodiments can be obtained by in vitro transcription. In particular embodiments, in vitro RNA transcripts are synthesized using a T7 RNA polymerase to generate RNA having one or more or all uridines substituted with a non-natural nucleotide, such as N¹-methyl pseudouridine (N1Mψ), a cap (provided by for example, a Cap-AG reagent), a poly(A) tail of approximately 90-110 nucleotides was present in the template plasmid. In some embodiments, RNA molecules generated by in vitro transcription are encapsulated in a LNP or tLNP, which LNPs or tLNPs can be used to transfect cells in vitro, in vivo, extracorporeally, or ex vivo.

(II)(a) Capped mRNA

Eukaryotic mRNAs have a 7-methylguanosine (m⁷G) cap added to their 5′ end through a 5′ to 5′ triphosphate bridge (Cap0). Additionally, the 2′ hydroxyl of the ribose of the first one or two templated nucleotides of the mRNA can be methylated (Cap1 and Cap2, respectively). Capping is a post-transcriptional event in vivo but can be either post- or co-transcriptional in vitro. In co-transcriptional capping an oligonucleotide comprising m⁷G and the first one, two, or more templated nucleotides, which hybridize to the template, is used as a primer. Two or more templated nucleotides that are not the same can be included to assure unidirectional transcription. Cap primers with a variety of templated nucleotides and with additional modifications are commercially available and/or described in the literature. Embodiments of the mRNAs provided herein can incorporate any of these cap structures or primers. Particular instances incorporate the primer m⁷G(5′)ppp(5′)(2′OMeA)pG (Cap AG). Cap AG is well-suited for capping of m RNA encoded by templates using a promoter resembling the T7 ϕ2.5 promoter in having the first two templated nucleotides of the mRNA have the sequence AG. Alternatives to Cap AG include m⁷G(UNA)(5′)ppp(5′)(2′OMeA)pG (with an “unlocked” structure for the first cap nucleoside, having no C—C bond between the carbons corresponding to the 2′ and 3′ positions) and m⁷G(5′)(ENE)ppp(5′)(2′OMeA)pG (with a double-bonded C replacing the 5′ O between the first cap nucleoside and the triphosphate) (Cap4011 and Cap5011, respectively, Areterna, Gaithersburg, MD). A further alternative is m^{7(LNA)}G(5′)ppp(5′)(2′OMeA)pG (Cap AG) (with an “locked” structure for the first cap nucleoside) (ThermoFisher PPT1647 0522).

In certain embodiments of the above aspects, an mRNA of this disclosure further comprises a co-transcriptional capping structure. Examples of the co-transcriptional capping structures includes without limitation, Cap0, ARCA (Anti-Reverse Cap Analog, a Cap0 mimic), Cap1, or Cap2 (Muttach et al., 2017, J Org Chem. 13: 2819-2832).

A template DNA molecule encoding the mRNA will further comprise a promoter upstream of the coding sequence appropriate to the DNA polymerase to be used for in vitro transcription. In vitro transcription is typically carried out using a phage DNA polymerase, such as T7, T3, or SP6 polymerase.

(II)(b) Poly(A) Tail of mRNA

The poly(A) tails of mRNAs produced in living cells are enzymatically added at a poly(A) addition site in the 3′ UTR following transcription and are generally around 200 residues in length. For in vitro transcribed mRNA the necessary enzymes can be added, but alternatively a poly(A) sequence can be encoded in the DNA template. Such poly(A) tails are often in the range of 90-110 nucleotides, but there are numerous examples in the scientific and patent literature advocating various modifications of length or sequence. Poly(A) tails in a population of mRNA molecules will be heterogenous in length even when templated due to slippage during replication of the template or during transcription.

(II)(c) Untranslated Regions (UTRs) of mRNA

The untranslated regions flanking the coding sequence of an mRNA are known to influence expression. For 5′ UTRs, it is known that excessive or insufficient length, high GC content—contributing to secondary structure formation—and a large number of AUG codons will negatively impact protein expression. Similarly, excessive 3′ UTR length can negatively impact protein expression. But beyond the avoidance of such features, how to choose or alter UTRs to improve protein expression and how 5′ and 3′ UTRs interact are not well understood. It is also known that the inclusion of microRNA (miRNA) binding sites in UTRs will alter expression in tissues that express the cognate miRNA, usually negatively. However, knowledge of which miRNAs are expressed in which tissues and what sequences they can functionally bind to is incomplete. Nonetheless, for known miRNAs with at least partially known expression profiles, it is possible to engineer a binding site into a UTR to suppress expression in a tissue in which expression is not desired (a non-target tissue) thereby conferring a degree of tissue specificity on expression. This miRNA-mediated specificity can augment specificity obtained from targeted delivery of an mRNA, such as with a tLNP or tropic LNP.

Untranslated regions (UTRs) and codon usages have been shown to modulate mRNA stability and translation activity. Various combinations of UTRs and codon usage in mRNA encoding a CAR were tested to screen mRNAs having an improved level of expression after transfected into a mammalian cell via tLNP delivery.

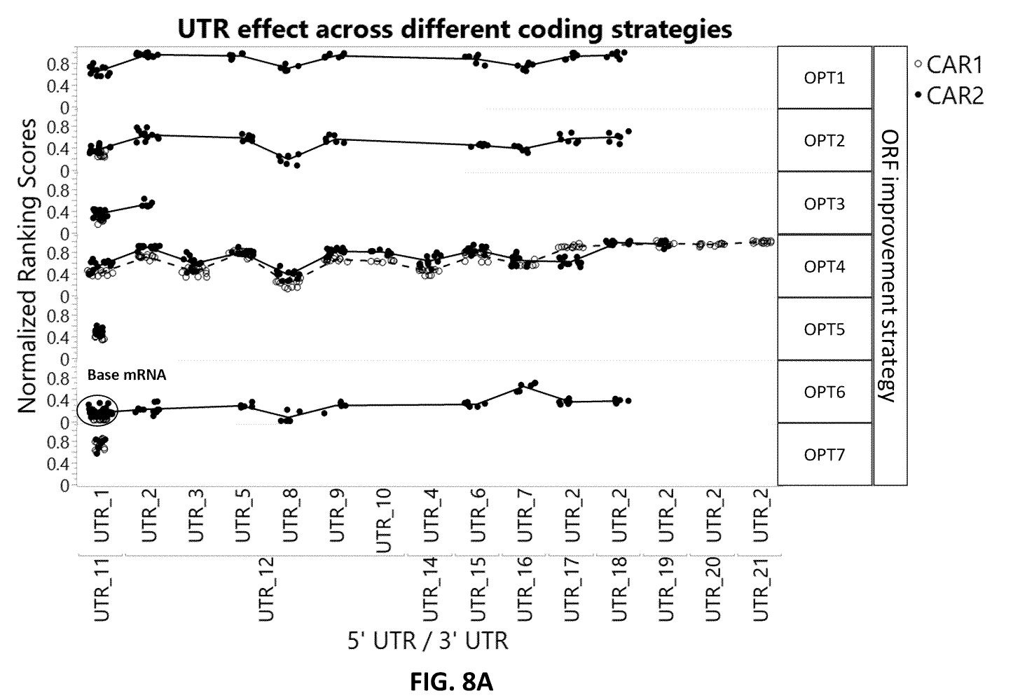

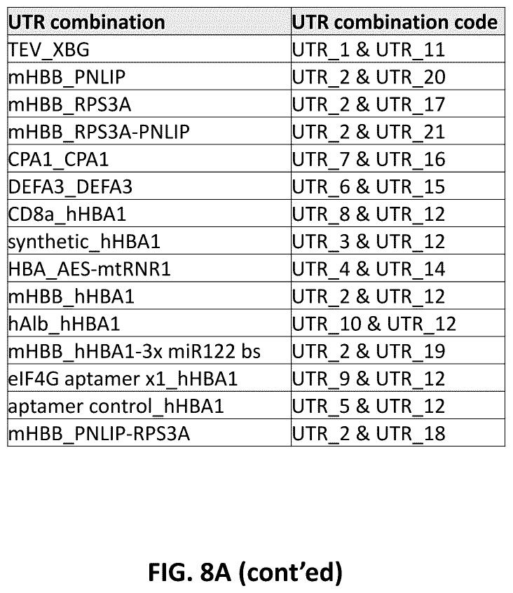

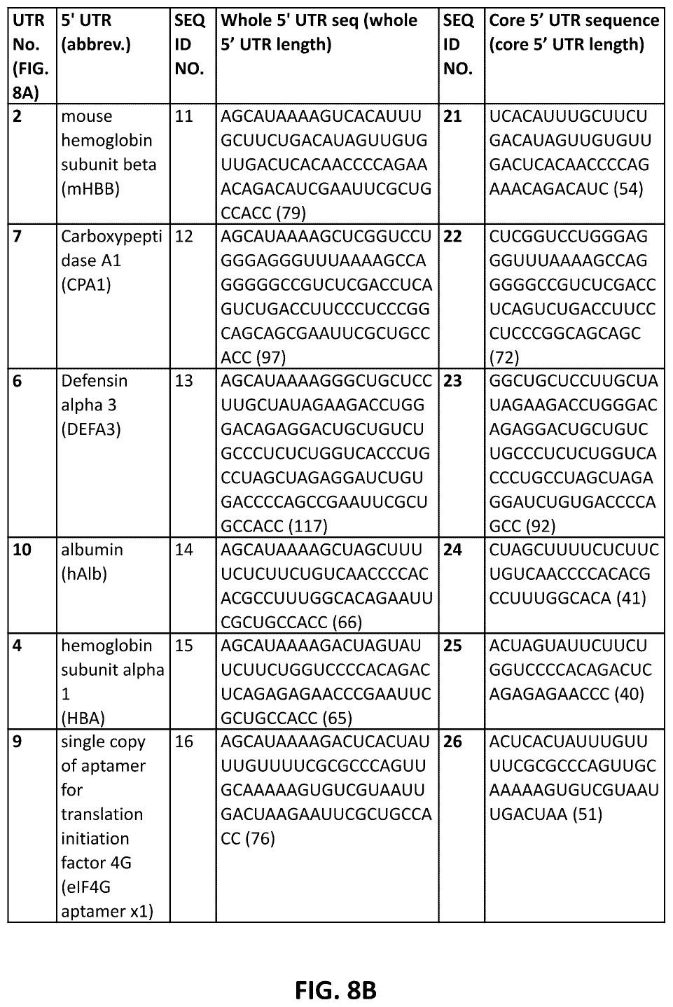

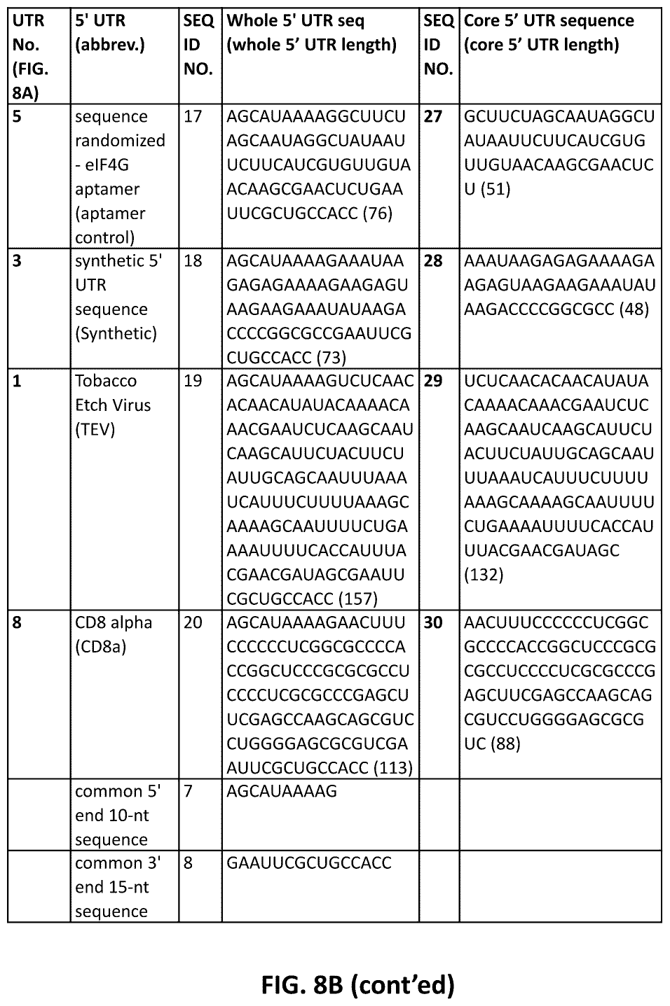

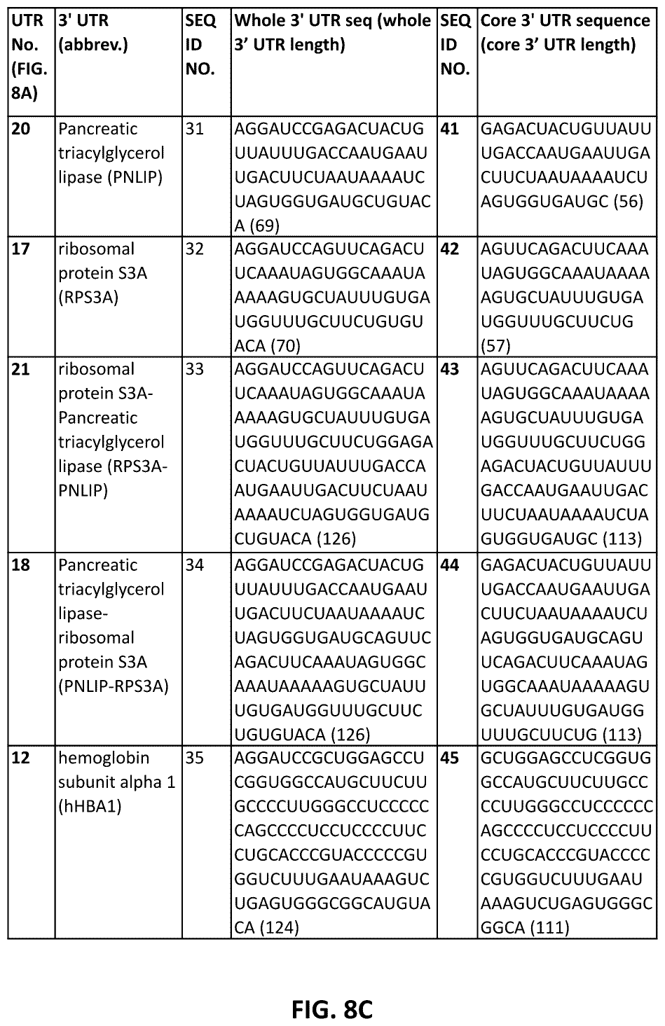

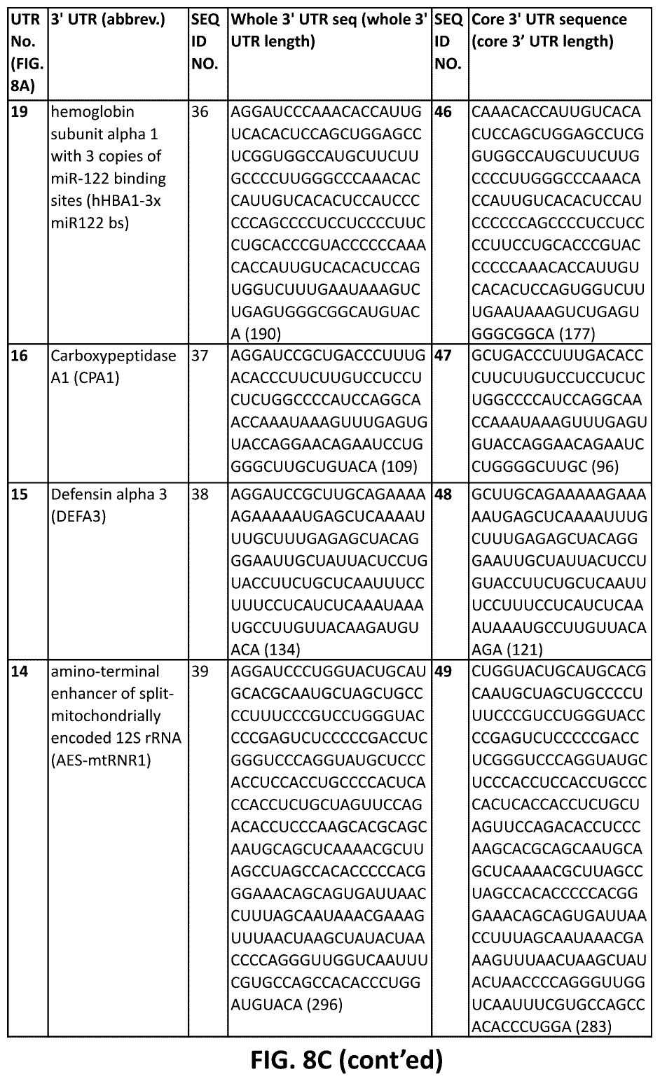

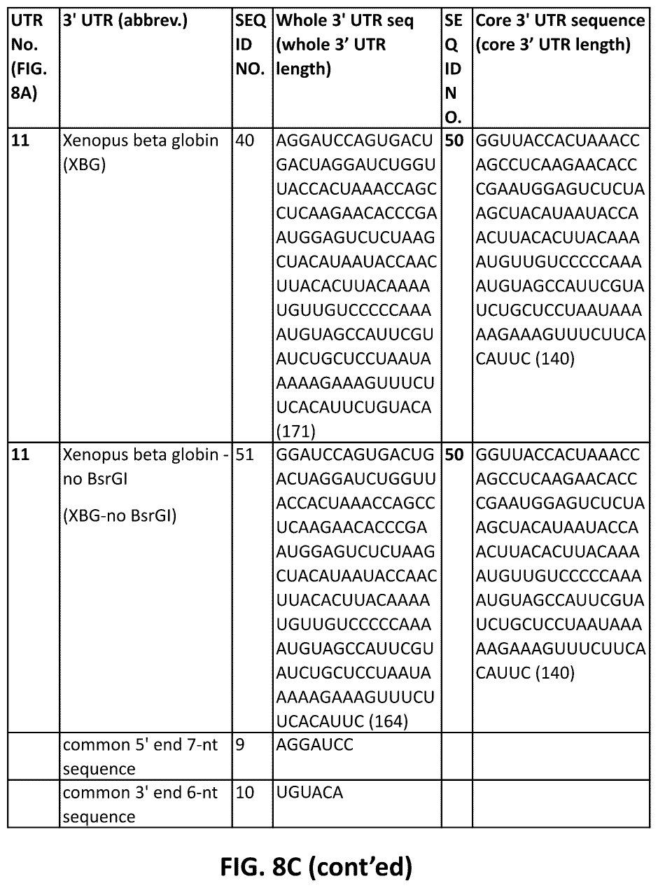

The 5′ UTRs used in the disclosed mRNAs comprise a unique core sequence flanked by a shared sequences at the 5′ and 3′ ends of the 5′ UTR. The shared 5′ end of the disclosed mRNAs is a ten-nucleotide sequence, AGCAUAAAAG (SEQ ID NO: 7), the initial AG conferring compatibility with the co-translational capping reagent m7G(5′)ppp(5′)(2′OMeA)pG. The shared 3′ end of the 5′ UTRs of the disclosed mRNAs is a 15-nucleotide sequence, GAAUUCGCUGCCACC (SEQ ID NO: 8), providing a Kozak sequence at its 3′ end. The unique core sequences of the 5′ UTRs, contributing to the differences in protein expression from the various mRNAs, are from the 5′ UTRs of carboxypeptidase A1 (CPA1) (SEQ ID NO: 22), defensin alpha 3 (DEF3A) (SEQ ID NO: 23), CD8 alpha (CD8a) (SEQ ID NO: 30), mouse hemoglobin subunit beta (mHBB) (SEQ ID NO: 21), a synthetic sequence (SEQ ID NO: 28), hemoglobin subunit alpha 1 (HBA) (SEQ ID NO: 25), tobacco etch virus (TEV) (SEQ ID NO: 29), a single copy of an aptamer for eukaryotic translation initiation factor 4G (eIF4G aptamer ×1) (SEQ ID NO: 26), a randomized sequence of the eIF4G aptamer (aptamer control) (SEQ ID NO: 27), and human albumin (hAlb) (SEQ ID NO: 24). UTRs comprising these core sequences are referred to by these names throughout this disclosure. All of these sequences are presented in FIG. 8B.

The 3′ UTRs used in the disclosed mRNAs comprise a unique core sequence flanked by shared sequences at the 5′ and 3′ ends of the 3′ UTR. The shared 5′ end of the 3′ UTRs of the disclosed mRNAs is a seven-nucleotide sequence, AGGAUCC (SEQ ID NO: 9). The shared 3′ end of the 3′ UTRs of the disclosed mRNAs is a six-nucleotide sequence, UGUACA (SEQ ID NO: 10). The Xenopus beta globin 3′ UTR is an exception having additional vector sequence in between the shared 5′ sequence and core sequence and in some instances is missing the initial A of the shared segment. The unique core sequences of the 3′ UTRs, contributing to the differences in protein expression from the various mRNAs, are from the 3′ UTRs of pancreatic triacylglycerol lipase (PNLIP) (SEQ ID NO: 41), ribosomal protein S3A (RPS3A) (SEQ ID NO: 42), carboxypeptidase A1 (CPA1) (SEQ ID NO: 47), defensin alpha 3 (DEFA3) (SEQ ID NO: 48), human hemoglobin subunit alpha 1 (hHBA1) (SEQ ID NO: 45), amino-terminal enhancer of split-mitochondrially encoded 12S rRNA (AES-mtRNR1) (SEQ ID NO: 49), human hemoglobin subunit alpha 1 with 3 copies of miR-122 binding sites (hHBA1-3× miR122 bs) (SEQ ID NO: 46), Xenopus beta globin (XBG) (SEQ ID NO: 50), and tandem arrays in either order of pancreatic triacylglycerol lipase and ribosomal protein S3A 3′ UTRs (PNLIP-RPS3A and RPS3A-PNLIP) (SEQ ID NOs: 44 and 43, respectively). UTRs comprising these core sequences are referred to by these names throughout this disclosure. All of these sequences are presented in FIG. 8C.

The sequences adjoining the UTR core sequences can be altered without disrupting the beneficial effect of the UTR core sequences. In altering these adjoining sequences compatibility with the capping system being used should be maintained as should the functionality of the Kozak sequence. The UTR-adjoining sequences should not form secondary structure within these adjoining sequences or with the core sequence or other adjoining sequence. This can be facilitated by using AU-rich sequences and avoiding complementarity with other portions of the UTR. Additionally, the 5′ UTR adjoining sequences should not contain a start (AUG) codon. Accordingly, some embodiments specify only the core sequences of the UTRs while other embodiments comprise whole UTR sequences, as disclosed herein.

In addition to linear mRNA, a gene of interest (GOI) can be expressed from other RNA modalities, including but not limited to circular RNAs and self-amplifying RNA (also known as self-replicating RNA or RNA replicon).

Circular RNA (circRNA) is an RNA molecule without RNA termini, and it does not contain a 5′-cap that is usually present in a linear synthetic mRNA molecule. Instead, circRNA needs at least one sequence moiety that recruits ribosomes where such moiety includes an internal ribosome entry site(s). The sequence of a gene of interest (GOI) is typically inserted downstream of the ribosome recruitment site to ensure efficient gene expression. Depending on the particular internal ribosome entry site used, the 5′ UTR can be wholly retained, just the Kozak sequence retained or be completely replaced by the internal ribosome entry site, Cell-free synthesis of circular RNA is initiated with in vitro transcription to produce a linear RNA followed by a variety of circularization reactions that involve: 1) a ribozyme, such as a self-splicing group I/II intron, 2) a protein enzyme like T4 DNA ligase or RNA ligase 1/2, with or without a split oligonucleotide molecule bridging the RNA termini, or 3) Non-enzymatic ligation, such as click chemistry (reviewed in Obi and Chen, The design and synthesis of circular RNAs. Methods. 2021 December; 196:85-103. doi: 10.1016/j.ymeth.2021.02.020. Epub 2021 Mar. 2. PMID: 33662562; PMCID: PMC8670866, which is incorporated by reference for all that it teaches about the design of circular RNA that is not inconsistent with this disclosure). Due to the circular nature of the final RNA molecule, there is a degree of flexibility on the placement of the 3′ elements and the circularization elements relative to the GOI in the linear precursor RNA.

Self-amplifying RNA (saRNA), on the other hand, is generated as a linear synthetic RNA molecule with a viral origin chassis that enables its replication within cells. The self-replication machinery encoded in the RNA backbone can be derived from a wide range of single-stranded positive or negative-sense RNA viruses. For instance, a genomic RNA backbone of alphavirus, a positive-stranded RNA virus, is commonly used as an saRNA platform to express an GOI, often for a vaccine application (reviewed in Bloom et al., Self-amplifying RNA vaccines for infectious diseases. Gene Ther. 2021 April; 28(3-4):117-129. doi: 10.1038/s41434-020-00204-y. Epub 2020 Oct. 22. PMID: 33093657; PMCID: PMC7580817, which is incorporated by reference for all that it teaches about the design of saRNA that is not inconsistent with this disclosure). In a typical alphavirus saRNA, the viral structural genes originally present in the alphavirus genome are usually removed and replaced by the GOI(s). Consequently, due to the absence of functional virion formation, the RNA replication remains confined within the cells where it is originally delivered. The non-structural replicase genes, including RNA-dependent RNA polymerase gene, in the alphavirus saRNA are directly expressed from the synthetic genomic transcripts to initiate the replication cycle in cells. In contrast, while encoded in the genomic transcript, a GOI(s) in alphavirus saRNA system is expressed from a subgenomic RNA, which is produced in abundance during RNA replication in cells. In a typical arrangement the viral nonstructural genes and subgenomic promoter (in that order) would be inserted between the 5′ UTR and the GOI of the linear mRNA. Alternatively, the 5′ UTR of the linear mRNA can largely replace the subgenomic promoter. The 3′ UTR of the linear mRNA can be retained but the viral promoter for minus strand synthesis must also be present. The viral non-structural genes can contain essential RNA structures that need to be retained by any codon optimization.

In certain aspects, this disclosure provides an mRNA having a sequence comprising in 5′ to 3′ order, a 5′ untranslated region (UTR), an open reading frame (ORF) terminated by at least one stop codon, a 3′ UTR, and a poly(A) sequence wherein the 5′ UTR and 3′ UTR each comprise a sequence, or a variant thereof having ≥95% sequence identity to the UTR sequence, selected from one of the following pairs:
a) mouse β-globin (mHBB) 5′ UTR (SEQ ID NO: 21) and pancreatic triacylglycerol lipase (PNLIP) 3′ UTR (SEQ ID NO: 41), ribosomal protein S3A (RPS3A) 3′ UTR (SEQ ID NO: 42), RPS3A-PNLIP tandem 3′ UTR (SEQ ID NO: 43), PNLIP-RPS3A tandem 3′ UTR (SEQ ID NO: 44), human hemoglobin subunit α1 (hHBA1) 3′ UTR (SEQ ID NO: 45) or human hemoglobin subunit α1 with 3 miRNA122 binding sites (hHBA1-3× miR122 bs) 3′ UTR (SEQ ID NO: 46);
b) carboxypeptidase A1 (CPA1) 5′ UTR (SEQ ID NO: 22) and CPA1 3′ UTR (SEQ ID NO: 47) wherein the ORF does not encode CPA1 or a protein having ≥60% sequence identity with CPA1;
c) defensin alpha 3 (DEFA3) 5′ UTR (SEQ ID NO: 23) and DEFA3 3′ UTR (SEQ ID NO: 48) wherein the ORF does not encode DEFA3 or a protein having ≥60% sequence identity with DEFA3;
d) human albumin (hAlb) 5′ UTR (SEQ ID NO: 24) and hHBA1 3′ UTR (SEQ ID NO: 45);
e) eIF4G aptamer ×1 (SEQ ID NO: 26) and hHBA1 3′ UTR (SEQ ID NO: 45); or
f) aptamer control (SEQ ID NO: 27) and hHBA1 3′ UTR (SEQ ID NO: 45);
   or a nucleic acid molecule comprising a DNA sequence encoding said mRNA.
   In some embodiments, the 5′ UTR of an mRNA comprising a 5′ UTR core sequence as disclosed herein further comprises SEQ ID NO: 7 at the 5′ end of the 5′ UTR and/or SEQ ID NO: 8 at the 3′ end of the 5′ UTR. In some embodiments, the 3′ UTR of an mRNA comprising a 3′ UTR core sequence as herein disclosed further comprises SEQ ID NO: 9 at the 5′ end of the 3′ UTR and/or SEQ ID NO: 10 at the 3′ end of the 3′ UTR.

In certain aspects, this disclosure provides an mRNA having a sequence comprising in 5′ to 3′ order, a 5′ untranslated region (UTR), an open reading frame (ORF) terminated by at least one stop codon, a 3′ UTR, and a poly(A) sequence wherein:
the ORF encodes a CAR; and
the 5′ UTR and 3′ UTR each comprise a sequence, or a variant thereof having ≥95% sequence identity to the UTR sequence, selected from one of the following pairs:
a) mouse β-globin (mHBB) 5′ UTR (SEQ ID NO: 21) and pancreatic triacylglycerol lipase (PNLIP) 3′ UTR (SEQ ID NO: 41), ribosomal protein S3A (RPS3A) 3′ UTR (SEQ ID NO: 42), RPS3A-PNLIP tandem 3′ UTR (SEQ ID NO: 43), PNLIP-RPS3A tandem 3′ UTR (SEQ ID NO: 44), human hemoglobin subunit α1 (hHBA1) 3′ UTR (SEQ ID NO: 45) or human hemoglobin subunit α1 with 3 miRNA122 binding sites (hHBA1-3× miR122 bs) 3′ UTR (SEQ ID NO: 46);
b) carboxypeptidase A1 (CPA1) 5′ UTR (SEQ ID NO: 22) and CPA1 3′ UTR (SEQ ID NO: 47) wherein the ORF does not encode CPA1 or a protein having ≥60% sequence identity with CPA1;
c) defensin alpha 3 (DEFA3) 5′ UTR (SEQ ID NO: 23) and DEFA3 3′ UTR (SEQ ID NO: 48) wherein the ORF does not encode DEFA3 or a protein having ≥60% sequence identity with DEFA3;
d) human albumin (hAlb) 5′ UTR (SEQ ID NO: 24) and hHBA1 3′ UTR (SEQ ID NO: 45);
e) hemoglobin subunit alpha 1 (HBA) 5′ UTR (SEQ ID NO: 25) and amino-terminal enhancer of split and mitochondrially encoded 12S rRNA(AES-mtRNR1) 3′ UTR (SEQ ID NO: 49);
f) eIF4G aptamer ×1 (SEQ ID NO: 26) and hHBA1 3′ UTR (SEQ ID NO: 45);
g) aptamer control (SEQ ID NO: 27) and hHBA1 3′ UTR (SEQ ID NO: 45); or
h) a synthetic 5′ UTR (SEQ ID NO: 28) and hHBA1 3′ UTR (SEQ ID NO: 45);
   or a nucleic acid molecule comprising a DNA sequence encoding said mRNA.

In some embodiments, the 5′ UTR of an mRNA comprising a 5′ UTR core sequence as disclosed herein further comprises SEQ ID NO: 7 at the 5′ end of the 5′ UTR and/or SEQ ID NO: 8 at the 3′ end of the 5′ UTR. In some embodiments, the 3′ UTR of an mRNA comprising a 3′ UTR core sequence as herein disclosed further comprises SEQ ID NO: 9 at the 5′ end of the 3′ UTR and/or SEQ ID NO: 10 at the 3′ end of the 3′ UTR.

In certain aspects, this disclosure provides a nucleic acid molecule, an mRNA or a nucleic acid molecule encoding the mRNA having a sequence comprising in 5′ to 3′ order, a 5′ untranslated region (UTR), an open reading frame (ORF) terminating with at least one stop codon, a 3′ UTR, and a poly(A) sequence, wherein the 5′ UTR and 3′ UTR each comprise a sequence, or a variant thereof having ≥95% sequence identity to the UTR sequence, selected from one of the following pairs:
a) mouse β-globin (mHBB) 5′ UTR (SEQ ID NO: 11) and pancreatic triacylglycerol lipase (PNLIP) 3′ UTR (SEQ ID NO: 31), ribosomal protein S3A (RPS3A) 3′ UTR (SEQ ID NO: 32), RPS3A-PNLIP tandem 3′ UTR (SEQ ID NO: 33), PNLIP-RPS3A tandem 3′ UTR (SEQ ID NO: 34), or human hemoglobin subunit α1 with 3 miRNA122 binding sites (hHBA1-3× miR122 bs) 3′ UTR (SEQ ID NO: 36);
b) carboxypeptidase A1 (CPA1) 5′ UTR (SEQ ID NO: 12) and CPA1 3′ UTR (SEQ ID NO: 37);
c) defensin alpha 3 (DEFA3) 5′ UTR (SEQ ID NO: 13) and DEFA3 3′ UTR (SEQ ID NO: 38);
d) human albumin (hAlb) 5′ UTR (SEQ ID NO: 14) and hHBA1 3′ UTR (SEQ ID NO: 35);
e) hemoglobin subunit alpha 1 (HBA) 5′ UTR (SEQ ID NO: 15) and amino-terminal enhancer of split and mitochondrially encoded 12S rRNA (AES-mtRNR1) 3′ UTR (SEQ ID NO: 39);
f) eIF4G aptamer ×1 5′ UTR (SEQ ID NO: 16) and hHBA1 3′ UTR (SEQ ID NO: 35);
g) aptamer control 5′ UTR (SEQ ID NO: 17) and hHBA1 3′ UTR (SEQ ID NO: 35); or
h) a synthetic 5′ UTR (SEQ ID NO: 18) and hHBA1 3′ UTR (SEQ ID NO: 35);
or a nucleic acid molecule comprising a DNA sequence encoding said mRNA.

In certain aspects, this disclosure provides an mRNA having a sequence comprising in 5′ to 3′ order, a 5′ untranslated region (UTR), an open reading frame (ORF) terminated by at least one stop codon, a 3′ UTR, and a poly(A) sequence wherein:
the ORF encodes a CAR; and
the 5′ UTR and 3′ UTR each comprise a sequence, or a variant thereof having ≥95% sequence identity to the UTR sequence, selected from one of the following pairs:
a) mouse β-globin (mHBB) 5′ UTR (SEQ ID NO: 11) and pancreatic triacylglycerol lipase (PNLIP) 3′ UTR (SEQ ID NO: 31), ribosomal protein S3A (RPS3A) 3′ UTR (SEQ ID NO: 32), RPS3A-PNLIP tandem 3′ UTR (SEQ ID NO: 33), PNLIP-RPS3A tandem 3′ UTR (SEQ ID NO: 34), or human hemoglobin subunit α1 with 3 miRNA122 binding sites (hHBA1-3× miR122 bs) 3′ UTR (SEQ ID NO: 36);
b) carboxypeptidase A1 (CPA1) 5′ UTR (SEQ ID NO: 12) and CPA1 3′ UTR (SEQ ID NO: 37) wherein the ORF does not encode CPA1 or a protein having ≥60% sequence identity with CPA1;
c) defensin alpha 3 (DEFA3) 5′ UTR (SEQ ID NO: 13) and DEFA3 3′ UTR (SEQ ID NO: 38) wherein the ORF does not encode DEFA3 or a protein having ≥60% sequence identity with DEFA3;
d) human albumin (hAlb) 5′ UTR (SEQ ID NO: 14) and hHBA1 3′ UTR (SEQ ID NO: 35);
e) hemoglobin subunit alpha 1 (HBA) 5′ UTR (SEQ ID NO: 15) and amino-terminal enhancer of split and mitochondrially encoded 12S rRNA(AES-mtRNR1) 3′ UTR (SEQ ID NO: 39);
f) eIF4G aptamer ×1 5′ UTR (SEQ ID NO: 16) and hHBA1 3′ UTR (SEQ ID NO: 35);
g) aptamer control 5′ UTR (SEQ ID NO: 17) and hHBA1 3′ UTR (SEQ ID NO: 35); or
h) a synthetic 5′ UTR (SEQ ID NO: 18) and hHBA1 3′ UTR (SEQ ID NO: 35); or a nucleic acid molecule comprising a DNA sequence encoding said mRNA.

In certain embodiments comprising CPA1 UTRs, the ORF does not encode CPA1 or a protein having ≥60% sequence identity with CPA1, wherein the CPA sequence is Genbank KAI4015818.1 (SEQ ID NO: 89). In certain embodiments comprising DEFA3 UTRs, the ORF does not encode DEFA3 or a protein having ≥60% sequence identity with DEFA3, wherein the DEFA3 sequence is Genbank AAI19707.1 (SEQ ID NO: 110). In certain embodiments the UTRs are heterologous to the encoded polypeptide. In certain embodiments, the ORF encodes a CAR. In other embodiments, the ORF encodes an immune cell engager such as a BiTE (Bispecific T cell Engager). In some embodiments, the ORF encodes only human or artificial sequences. With respect to these and other nucleic acid aspects, in some embodiments, the mRNA is suitable for expression in a mammalian cell. In some embodiments, the mammalian cell is an immune cell or a hematopoietic stem cell. In some embodiments, any of these cells is a human cell. These mRNAs constitute means for improved expression of a polypeptide, means for improved expression of a polypeptide in an immune cell, means for expression of a polypeptide in a T cell, and the like. Some embodiments are limited to a subset of one or more of the UTR pairings disclosed herein. Other embodiments specifically exclude one or more of these UTR pairings. These mRNAs constitute means for improved expression of a CAR, means for improved expression of a CAR in an immune cell, means for expression of a CAR in a T cell, and the like. Some embodiments are limited to a subset of one or more of these UTR pairings. Other embodiments specifically exclude one or more of these UTR pairings.

In certain embodiments, the CAR is an anti-CD19 CAR (e.g., without limitation, CAR1 or CAR2) or an anti-CD20 CAR (e.g., without limitation, CAR7, CAR22, or CAR25).

In certain aspects, this disclosure provides an mRNA having a sequence comprising in 5′ to 3′ order, a 5′ untranslated region (UTR), an open reading frame (ORF) terminated by at least one stop codon, a 3′ UTR, and a poly(A) sequence wherein: the 3′ UTR is pancreatic triacylglycerol lipase-ribosomal protein S3A (PNLIP-RPS3A) (SEQ ID NO: 44 or SEQ ID NO: 34) or ribosomal protein S3A-pancreatic triacylglycerol lipase (RPS3A-PNLIP) (SEQ ID NO: 43 or SEQ ID NO: 33).

In certain embodiments, mHBB as a 5′ UTR in combination with tandem format (PNLIP-RPS3A/RPS3A-PNLIP) or individual 3′ UTR of PNLIP or RPS3A, and hHBA1-3× miR122 bs 3′ UTR are the UTR pairings providing the greatest increase in expression of an ORF encoding a CAR.

In certain aspects, this disclosure provides an mRNA having a sequence comprising in 5′ to 3′ order, a 5′ UTR core sequence, an ORF terminated by at least one stop codon, a 3′ UTR, and a poly(A) sequence. In particular embodiments, the 5′ UTR core sequence is carboxypeptidase A1 (CPA1) (SEQ ID NO: 22), defensin alpha 3 (DEFA3) (SEQ ID NO: 23), mouse hemoglobin subunit beta (mHBB) (SEQ ID NO: 21), synthetic (SEQ ID NO: 28), human hemoglobin subunit beta (HBA) (SEQ ID NO: 25), human albumin (hAlb) (SEQ ID NO: 24), aptamer control (SEQ ID NO: 27), or eIF4G aptamer ×1 (SEQ ID NO: 26). In some embodiments, the 5′ UTR of an mRNA comprising a 5′ UTR core sequence as disclosed herein further comprises SEQ ID NO: 7 at the 5′ end of the 5′ UTR and/or SEQ ID NO: 8 at the 3′ end of the 5′ UTR. Some embodiments specifically include a subset of these 5′ UTR core sequences while other embodiments specifically exclude a subset of the herein disclosed 5′ UTR core sequences. Some embodiments further specifically include an ORF encoding a particular protein or type of protein as disclosed herein. In some embodiments, the 3′ UTR is not derived from the same gene as the 5′ UTR. Some embodiments further specifically include a 3′ UTR disclosed herein.

In certain aspects, this disclosure provides an mRNA having a sequence comprising in 5′ to 3′ order, a 5′ UTR, an ORF terminated by at least one stop codon, a 3′ UTR core sequence, and a poly(A) sequence wherein: the 3′ UTR core sequence comprises pancreatic triacylglycerol lipase (PNLIP) (SEQ ID NO: 41), ribosomal protein S3A (RPS3A) (SEQ ID NO: 42), pancreatic triacylglycerol lipase-ribosomal protein S3A (PNLIP-RPS3A) (SEQ ID NO: 44), ribosomal protein S3A-pancreatic triacylglycerol lipase (RPS3A-PNLIP) (SEQ ID NO: 43), amino-terminal enhancer of split and mitochondrially encoded 12S rRNA mouse hemoglobin subunit beta (AES-mtRNR1) (SEQ ID NO: 49), carboxypeptidase A1 (CPA1) (SEQ ID NO: 47), defensin alpha 3 (DEFA3) (SEQ ID NO: 48), human hemoglobin subunit alpha 1 (hHBA1) (SEQ ID NO: 45), or human hemoglobin subunit alpha 1 with 3 copies of miR-122 binding sites (hHBA1-3× miR122 bs) (SEQ ID NO: 46). In some embodiments, the 3′ UTR of an mRNA comprising a 3′ UTR core sequence as herein disclosed further comprises SEQ ID NO: 9 at the 5′ end of the 3′ UTR and/or SEQ ID NO: 10 at the 3′ end of the 3′ UTR. Some embodiments specifically include a subset of these 3′ UTR core sequences while other embodiments specifically exclude a subset of the herein disclosed 3′ UTR core sequences. Some embodiments further specifically include an ORF encoding a particular protein or type of protein as disclosed herein. In some embodiments, the 5′ UTR is not derived from the same gene as the 3′ UTR. Some embodiments further specifically include a 5′ UTR disclosed herein.

In certain aspects, this disclosure provides an mRNA having a sequence comprising in 5′ to 3′ order, a 5′ UTR, an ORF terminated by at least one stop codon, a 3′ UTR, and a poly(A) sequence wherein: the 5′ UTR comprises carboxypeptidase A1 (CPA1) (SEQ ID NO: 12), defensin alpha 3 (DEFA3) (SEQ ID NO: 13), mouse hemoglobin subunit beta (mHBB) (SEQ ID NO: 11), synthetic (SEQ ID NO: 18), human hemoglobin subunit beta (HBA) (SEQ ID NO: 15), human albumin (hAlb) (SEQ ID NO: 14), aptamer control (SEQ ID NO: 17), or eIF4G aptamer ×1 (SEQ ID NO: 16). Some embodiments specifically include a subset of these 5′ UTR sequences while other embodiments specifically exclude a subset of these 5′ UTR sequences. Some embodiments further specifically include an ORF encoding a particular protein or type of protein as disclosed herein. In some embodiments, the 3′ UTR is not derived from the same gene as the 5′ UTR. Some embodiments further specifically include a 3′ UTR disclosed herein.

In certain aspects, this disclosure provides an mRNA having a sequence comprising in 5′ to 3′ order, a 5′ untranslated region (UTR), an open reading frame (ORF) terminated by at least one stop codon, a 3′ UTR, and a poly(A) sequence wherein: the 3′ UTR comprises pancreatic triacylglycerol lipase (PNLIP) (SEQ ID NO: 31), ribosomal protein S3A (RPS3A) (SEQ ID NO: 32), pancreatic triacylglycerol lipase-ribosomal protein S3A (PNLIP-RPS3A) (SEQ ID NO: 34), ribosomal protein S3A-pancreatic triacylglycerol lipase (RPS3A-PNLIP) (SEQ ID NO: 33), amino-terminal enhancer of split and mitochondrially encoded 12S rRNA mouse hemoglobin subunit beta (AES-mtRNR1) (SEQ ID NO: 39), carboxypeptidase A1 (CPA1) (SEQ ID NO: 37), defensin alpha 3 (DEFA3) (SEQ ID NO: 38), human hemoglobin subunit alpha 1 (hHBA1) (SEQ ID NO: 35), or human hemoglobin subunit alpha 1 with 3 copies of miR-122 binding sites (hHBA1-3× miR122 bs) (SEQ ID NO: 36). Some embodiments specifically include a subset of these 3′ UTR sequences while other embodiments specifically exclude a subset of these 3′ UTR sequences. Some embodiments further specifically include an ORF encoding a particular protein or type of protein as disclosed herein. In some embodiments, the 5′ UTR is not derived from the same gene as the 3′ UTR. Some embodiments further specifically include a 5′ UTR disclosed herein.

The 5′ UTR aptamer sequence eIF4G aptamer ×1 is originally disclosed in the PCT Application No. PCT/EP2018/078794, and the sequence and its synthesis are incorporated by reference. The 5′ UTR synthetic sequence is disclosed in U.S. Pat. No. 10,881,730, and the sequence and its synthesis are incorporated by reference. The 3′ UTR aptamer sequence hHBA1 3× miR122 bs is from U.S. patent application Ser. No. 14/903,869, and the sequence and its synthesis are incorporated by reference. The 3′ UTR AES-mtRNR1 is previously described in Orlandini von Niessen, A. G. et al., 2019, Mol. Ther. 27, 824-836 and the sequence and its synthesis are incorporated by reference.

### (II)(d) Opening Reading Frame (ORF)

The term “open reading frame” (ORF) refers to a nucleic acid sequence that begins with a start codon and is immediately followed by a stop codon. ORF can encode any protein. For engineered mRNAs, the codons can be those naturally found in mRNA encoding a particular protein or segment of a protein, the codons can be derived from back-translation of the amino acid sequence of a protein without reference to a natural sequence, and in either case these sequences can be subjected to various codon optimizations to favor particular subsets of synonymous codons, adjust GC content, avoid detrimental secondary structures, reduce the occurrence of ribosomal slippery sites (Mulroney et al., 2023, Nature 625: 189-194), and the like, in order to increase the level of translation in a particular species or type of cell. Especially when an mRNA is to be administered to a mammal, it can be advantageous to incorporate one or more modified nucleotides that, while maintaining coding function, increase translation and avoid provoking an innate immune response. In some embodiments, the replaced nucleoside is uridine. In certain embodiments all of the uridines are substituted with a modified nucleoside. In some embodiments, the modified nucleoside is pseudouridine (ψ), N¹-methylpseudouridine (N1 Mψ), 5-methoxyuridine (5moU), 5-methylcytidine, 5-methyluridine, N⁶-methyladenosine, 2′-O-methyluridine, or 2-thiouridine, or combinations thereof. Further disclosure of modified nucleosides and their use can be found in U.S. Pat. No. 8,278,036 which is incorporated herein by reference for those teachings. Natural protein-coding open reading frames are immediately followed by a single stop codon however, it is not uncommon for the open reading frame of engineered mRNAs to be followed by multiple in-frame stop codons.

### (II)(d)(1) Codon Optimization Methods

In certain embodiments, the ORF has been subject to codon optimization (which accommodates variations in tRNA populations for amino acids having multiple alternative codons) according to methods known in the art. Codon optimization refers to approaches designed to improve the codon composition of a recombinant gene based on various criteria without altering the amino acid sequence. This is possible because most amino acids are encoded by more than one codon. Most codon optimization methods avoid the use of rare codons and take into consideration other mRNA features that can inhibit expression such as mRNA instability elements, nucleotide context of the initiation codon, mRNA secondary structures, sequence repeats, nucleotide composition, internal ribosome entry sites, promoter sequences, and putative splice donor and acceptor sites (Mauro et al., 2014, Trends Mol Med. 20(11): 604-613; Gao et al., 2004, Biotechnology progress. 20:443-448; Raab et al., 2010, Systems and synthetic biology 4:215-225; Gaspar et al., 2012, Bioinformatics 28:2683-2684; Fath et al., 2011, PloS one 6:e17596). Many codon optimization methods randomly choose codons from a selection of codons that are generated by an algorithm based on selected key factors of gene transcription and translation (algorithmically equivalent codons). Note that because of this randomization of the algorithmically equivalent codons, multiple optimization attempts of the same sequence will generate different, algorithmically equivalent results.

Codon optimization methods described herein include a maximized codon adaptation index for human (maxiCAI) (OPT4, see Table 1) which is a “one amino acid-one codon” method as described in Pesole et al., 1988, NAR 16:1715-1728. See also Sharp & Li, 1987 NAR 15:1281-1295. As disclosed herein, human codon usage (referring to the non-random use of codons in mRNAs, quantified using various surveys) was taken from the Kazusa codon usage database (found on the website kazusa.or.jp/codon/). An online CAI calculator can be found at the website biologicscorp.com/tools/CAICalculator/. Additionally, other codon optimization methods include those provided by GeneArt, Genewiz, GenScript, and IDT. The GeneArt GeneOptimizer algorithm (OPT2, see Table 1) can be accessed through the website thermofisher.com/us/en/home/life-science/cloning/gene-synthesis/geneoptimizer.html. As described there, the algorithm takes into account GC content, consensus and cryptic splice sites, Shine-Dalgarno sequences, TATA boxes, termination signals and artificial recombination sites, all of which can affect transcription, RNA instability motifs, ribosomal entry sites, and repetitive sequences. All of which can also affect the stability and functionality of the mRNA, and codon usage, premature polyadenylation sites, ribosomal entry sites, and secondary structures, all of which can affect translation.

The Genewiz codon optimization tool (OPT3, see Table 1) can be accessed through the website genewiz.com/Public/Services/Gene-Synthesis/Codon-Optimization. This algorithm replaces low-frequency codons, avoids inverted repeats in the sequence that might form secondary structures inhibiting translation, and depletes CpG dinucleotides (as described in the Tech Note Enhancing Protein Expression by Leveraging Codon Optimization published by Azenta Life Sciences).

The GenScript GenSmart™ codon optimization tool (OPT8, see Table 1) can be accessed through https://www.genscript.com/gensmart-free-gene-codon-optimization.html. As described there, the tool is an implementation of the algorithm disclosed in WO2020021917 which takes into consideration over 200 factors influencing gene expression based on both population genetics and immunology theories including GC content, codon usage and content index, RNase splicing sites, and cis-acting mRNA destabilizing motifs.

The IDT codon optimization tool (OPT6) can be accessed through the websiteidtdna.com/pages/tools/codon-optimization-tool. As described there, the algorithm lowers complexity and minimizes secondary structures along with avoiding rare codons and rebalancing codon usage in accord with the species the protein will be expressed in.

Other codon optimization strategies such as CAR1 and CAR2 native versions (OPT7 and OPT1) are based on published sequences of CAR1 and CAR2 with limited manual codon selections primarily in the scFv portion of the CAR. The NIH method (OPT5, see Table 1) is based on Genbank nucleotide sequences, MN698642 and MN702882 for CAR1 and CAR2, respectively, which appear to be codon optimized.

Table 1 summarizes the codon optimization methods and the corresponding OPT codes mentioned throughout the application. The protein encoded by the ORF will be followed by the optimization code given in Table 1, for example, CAR1 sequence optimized by GeneArt will be called CAR1-OPT2

TABLE 1
Summary of ORF improvement strategies.

| OPT | Improvement | |
|---|---|---|
| code | Strategy | Note on ORF improvement strategies |
| OPT0 | N/A | No codon optimization |
| OPT1 | Native ver2 | Manual codon selections from known Genbank |
| | | sequences |
| OPT2 | GeneART | GeneArt's GeneOptimizer Algorithm |
| OPT3 | Genewiz | Genewiz algorithm |
| OPT4 | MaxiCAI | Algorithm based on Pesole et al., 1988, |
| | | NAR 16: 1715-1728. See also Sharp & Li, |
| | | 1987, NAR 15: 1281-1295. |
| OPT5 | NIH | Genbank nucleotide sequences, MN698642 and |
| | | MN702882 for CAR1 and CAR2, respectively, |
| | | which appear to be codon optimized. |
| OPT6 | IDT | IDT algorithm |
| OPT7 | Native ver1 | Manual codon selections from known Genbank |
| | | sequences |
| OPT8 | GenScript | GenScript's GenSmart Algorithm for human |

In certain embodiments, maxiCAI (OPT4), and native ver1 and ver2 (OPT7 and OPT1, respectively) are among the best performing mRNAs.

### (II)(d)(2) CAR Constructs

In some embodiments of the above aspects of the invention disclosed herein, an improved mRNA of this disclosure comprising an ORF encodes a chimeric antigen receptor (CAR). The receptors are chimeric because they combine into a single receptor both an antigen-binding domain of an antibody and one or more T cell activating domains of receptors expressed by T cells. In some embodiments, a CAR of this disclosure is multispecific, for example, bispecific, comprising multiple antigen binding moieties each specific for a separate antigen. In some embodiments, a CAR can comprise an extracellular binding domain that specifically binds a target antigen, a transmembrane domain, and one or more intracellular signaling domains. In some embodiments, a CAR can further comprise one or more additional elements, including one or more signal peptides, one or more extracellular hinge domains, or one or more intracellular costimulatory domains. Domains can be directly adjacent to one another, or there can be one or more amino acids linking the domains. The signal peptide can be derived from an antibody, a TCR, CD8 or other type 1 membrane protein, preferably a protein expressed in a T cell or other immune cell. The transmembrane domain can be one associated with any of the potential intracellular domains or from another type 1 membrane protein, such as TCR alpha, beta, or zeta chain, CD3 epsilon, CD4, CD8, or CD28, amongst other possibilities known in the art. The transmembrane domain can further comprise a hinge domain located between the extracellular binding domain and the hydrophobic membrane-spanning region of the transmembrane domain. In some but not all embodiments, the hinge domain and transmembrane domain are contiguous sequences in the same source protein. In some instances, the hinge and membrane-spanning domains are derived from CD28. In other instances, the hinge and membrane-spanning domains are derived from CD8a. In still other instances, the hinge domain is derived from an immunoglobulin, for example, the hinge domain of IgG4. The intracellular signaling domain can be derived from the CD3 zeta chain, DAP10, DAP12, FcγRIII, FcsRI, or an immunoreceptor tyrosine-based activation motif (ITAM) bearing cytoplasmic domain, amongst other possibilities known in the art. The intracellular costimulatory domain can be derived from CD27, CD28, 4-1BB, OX40, or ICOS, amongst other possibilities known in the art.

There are five generations of CARs that are commonly recognized. “First generation” CARs for use in the invention comprise an antigen binding domain, for example, a single-chain variable fragment (scFv) or VHH, fused to a transmembrane domain, which is fused to a cytoplasmic/intracellular domain of the T cell receptor chain. “First generation” CARs typically have the intracellular signaling (or activation) domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation. Use of a CD3ζ (intracellular signaling domain in which one or two of the three ITAM motifs has been disrupted can modulate the balance of effector and memory programs (Feucht et al., 2019 Nat Med 25(1):82-88). The intracellular signaling domains of CD3ε or the low affinity receptor for IgG, FcγRIIIA (CD16A) can be used as alternatives to CD3ζ. In some embodiments, the intracellular signaling domain of CD3ε comprises the sequence KNRKAKAKPVTRGAGAGGRQRGQNKERPPPVPNPDYEPIRKGQRDLYSGLNQRRI (SEQ ID NO: 227). In some embodiments, the intracellular signaling domain of FcγRIIIA (CD16A) comprises the sequence of FcγRIIIA: KTNIRSSTRDWKDHKFKWRKDPQDK (SEQ ID NO: 228). In some embodiments, these intracellular signaling domains constitute means for signaling or means for activation.

“Second-generation” CARs for use in the invention comprise an antigen binding domain, for example, a scFv or VHH, fused to a transmembrane domain, which is fused to an intracellular signaling domain capable of activating T cells and a co-stimulatory domain designed to augment T cell potency and persistence (Sadelain et al., 2013, Cancer Discov. 3:388-398). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1 BB, ICOS, OX40, CD27, and the like, in the cytoplasmic tail of the CAR to provide additional signals to the cell. “Second generation” CARs provide both co-stimulation, for example, by CD28 or 4-1 BB domains, and activation, for example, by a CD3ζ signaling domain. Preclinical studies have indicated that “Second Generation” CARs can improve the anti-tumor activity of CAR-T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., 2012, Oncoimmunol. 1(9):1577-1583). In some embodiments, these costimulatory domains constitute means for costimulation.

“Third generation” CARs provide multiple co-stimulation, for example, by comprising both CD28 and 4-1 BB domains, and activation, for example, by comprising a CD3ζ activation domain.

“Fourth generation” CARs provide co-stimulation, for example, by CD28 or 4-1 BB domains, and activation, for example, by a CD3ζ signaling domain in addition to a constitutive or inducible chemokine component.

“Fifth generation” CARs provide co-stimulation, for example, by CD28 or 4-1 BB domains, and activation, for example, by a CD3ζ signaling domain, a constitutive or inducible chemokine component, and an intracellular domain of a cytokine receptor, for example, IL-2Rβ.

Further variations on the basic CAR structure and sources for the various domains are described in Zabel et al., Immunol Lett 2019 212:53-69 which is incorporated by reference for all that it teaches about CAR structure and functional domains thereof to the extent it is consistent with this disclosure.

In some embodiments, a nucleic acid encoding a CAR refers to one or more nucleic acid species encoding one or more CARs; for example, a single or multiple species of nucleic acid encoding a single CAR species, or multiple species of nucleic acid encoding multiple CAR species. In some instances, these multiple CAR species have a same specificity while in other instances they have multiple specificities. In some embodiments, a CAR of this disclosure is multispecific, for example, bispecific, comprising multiple antigen binding moieties each specific for separate antigens.

### (II)(d)(2)(2a) Signal Peptide

In certain embodiments, the CAR can comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides

| SEQ | | |
|---|---|---|
| ID NO: | Sequence | Description |
| 107 | MALPVTALLLPLALLLHAARP | CD8α signal |
| | | peptide |
| 108 | METDTLLLWVLLLWVPGSTG | IgK signal |
| | | peptide |
| 109 | MLLLVTSLLLCELPHPAFLLIP | GMCSFR-α |
| | | (CSF2RA) signal |
| | | peptide |

### (II)(d)(2)(2b) Extracellular Binding Domain

A CAR comprises an extracellular binding domain, also referred to as a binder or binding moiety. In certain embodiments, the extracellular binding domain can comprise one or more antibodies specific to one target antigen or multiple target antigens. The antibody can be an antibody fragment, for example, an scFv, or a single-domain antibody fragment, for example, a VHH. In certain embodiments, the scFv can comprise a heavy chain variable region (V_{H}) and a light chain variable region (V_{L}) of an antibody connected by a linker. The V_{H} and the V_{L} can be connected in either order, i.e., V_{H}-linker-V_{L} or V_{L}-linker-V_{H}. Non-limiting examples of linkers include Whitlow linker, (G₄S)n (SEQ ID NO: 233, n can be a positive integer, e.g., 1, 2, 3, 4, 5, 6, etc.) linker, and variants thereof. In certain embodiments, the antigen can be an antigen that is exclusively or preferentially expressed on tumor cells, or an antigen that is characteristic of an autoimmune or inflammatory disease.

Exemplary target antigens against which a CAR, TCR, or ICE can have specificity include, but are not limited to, B cell maturation agent (BCMA)^{†‡}, CA9^{†‡}, CD4^{†‡}, CD5^{†‡}, CD19*^{†‡}, CD20 (MS4A1)*^{†‡}, CD22*^{†‡}, FCRL5^{†‡}, GPRC5D^{†‡}, CD23*^{†‡}, CD30 (TNFRSF8)*^{†‡}, CD33*^{†‡}, CD38*^{†‡}, CD44*^{‡}, CD70*^{†‡}, CD133^{‡}, CD174, CD274 (PD-L1)*^{†‡}, CD276 (B7-H3)^{†‡}, CEACAM5*^{†‡}, CLL1^{‡}, CSPG4*^{‡}, Kappa*, Lambda*, NCAM1 (CD56)*^{‡}, PD-1 (CD279)^{†‡}, ROR1^{†‡}, CD138 (SDC1)*^{‡}, CD319 (SLAMF7)*^{†‡}, CD248 (TEM1)^{‡}, ULBP1, and ULBP2 (associated with leukemias); CD319 (SLAMF7)*^{†‡}, CD38*^{†‡}, CD138^{†‡}, GPRC5D^{†‡}, CD267 (TACI)^{‡}, and BCMA^{†‡}, (associated with myelomas); and Claudin 6 (CLDN6), Claudin 18.2 (CLDN18.2), GD2*^{†‡}, HER2*^{†‡}, EGFR*^{†‡}, EGFRvIII*, CD276 (B7H3)^{†‡}, PSMA*^{†‡}, PSCA^{‡}, CAIX (CA9)^{†‡}, CD171 (L1-CAM)*^{‡}, CEA*^{‡}, CSPG4*^{‡}, DLL3, EPHA2*‡F, FAP*^{†‡}, LRRC15^{†‡}, FOLR1*^{†‡}, IL-13Rα^{†‡}, Mesothelin (MSLN)*^{†‡}, MUC1*^{†‡}, MUC16*^{†‡}, EPCAM*^{†‡}, ERBB2*^{‡}, FOLH1, GPC3*^{†‡}, GPNMB*^{‡}, IL1RAP^{†‡}, IL3RA*^{‡}, IL13RA2 (IL13Rα2)*^{‡}, KDR (VEGFR2)*^{‡}, CD171 (L1CAM)*^{‡}, MET*‡F, TROP2*^{†‡}, and ROR1^{†‡} (associated with solid tumors). Antigens associated with B cell leukemias can also be useful for B cell depletion in non-oncologic applications, however, CD19 (present on pro-B cells, pre-B cells, immature, naïve, germinal center, and memory B cells, and short-lived plasmablasts (sometime referred to as short-lived plasma cells)) and BCMA (present on memory B cells, short-lived plasmablasts, and long-lived plasma cells) are of particularly interest. (* indicates that exemplary antibodies with the indicated specificity from which a binding moiety could be derived can be found in U.S. Pat. No. 11,326,182B2 Table 9 or 10. ^{†} indicates that exemplary antibodies with the indicated specificity from which a binding moiety could be derived can be found in Wilkinson & Hale, 2022. Both references cited and incorporated by reference above. ^{‡} indicates that exemplary antibodies with the indicated specificity from which a binding moiety could be derived can be found in the Therapeutic Antibody Database (TABS) at tabs.craic.com). Other suitable antibodies can be found in Appendix A. Many of these target antigens are themselves receptors that could bind to their ligand if expressed on an immune cell. Accordingly, in some embodiments, the extracellular binding domain of the CAR comprises a ligand of a receptor expressed on the target cell. In still further embodiments, the extracellular binding domain of the CAR comprises a ligand binding domain of a receptor for a ligand expressed on the target cell. In any of these embodiments, the extracellular binding domain of the CAR can be codon-optimized for expression in a host cell or have variant sequences to increase functions of the extracellular binding domain. The advantages of the aspects and embodiments disclosed herein are independent of the specificity of the binding moiety. As such, the disclosed aspects and embodiments are generally agnostic to binding specificity. In certain embodiments, a particular binding specificity can be required. A more extensive discussion of antibodies recognizing many of the individual antigens listed above can be found in WIPO Publication WO2024040195A1 and U.S. patent application Ser. No. 18/731,223 which are each incorporated by reference for all that they teach about antibodies and related molecules that can be used to provide binding moieties recognizing target antigens.

### (II)(d)(2)(2c) Hinge Domain

In certain embodiments, the CAR can comprise a hinge domain, also referred to as a spacer. The terms “hinge” and “spacer” can be used interchangeably in this disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ | | |
|---|---|---|
| ID NO: | Sequence | Description |
| 230 | TTTPAPRPPTPAPTIASQPLSLRPEACRPA | CD8α hinge |
| | AGGAVHTRGLDFACD | domain |
| 231 | IEVMYPPPYLDNEKSNGTIIHVKGKHLCPS | CD28 hinge |
| | PLFPGPSKP | domain |
| 232 | ESKYGPPCPPCP | IgG4 hinge |
| | | domain |
| 234 | ESKYGPPCPPCPAPEFLGGPSVFLFPPKP | IgG4 hinge- |
| | KDTLMISRTPEVTCVVVDVSQEDPEVQFN | CH2-CH3 |
| | WYVDGVEVHNAKTKPREEQFNSTYRVVS | domain |
| | VLTVLHQDWLNGKEYKCKVSNKGLPSSIE | |
| | KTISKAKGQPREPQVYTLPPSQEEMTKNQ | |
| | VSLTCLVKGFYPSDIAVEWESNGQPENNY | |
| | KTTPPVLDSDGSFFLYSRLTVDKSRWQEG | |
| | NVFSCSVMHEALHNHYTQKSLSLSLGK | |

### (II)(d)(2)(2d) Transmembrane Domain

In certain embodiments, the CAR can comprise a transmembrane domain. In other embodiments, the transmembrane domain can comprise a transmembrane region of CD3ζ, CD3ε, CD3γ, CD3δ, CD4, CD5, CD8α, CD8β, CD9, CD16, CD22, CD28, CD32, CD33, CD34, CD37, CD40, CD45, CD64, CD80, CD86, OX40/CD134, 4-1BB/CD137, CD40L/CD154, FAS, FcεRIγ, FGFR2B, TCRα, TCRβ, or VEGFR2, or a functional variant thereof, including the human versions of each of these sequences. Table 4 provides the amino acid sequences of a few exemplary transmembrane domains.

TABLE 4
Exemplary sequences of transmembrane domains

| SEQ | | |
|---|---|---|
| ID NO: | Sequence | Description |
| 235 | IYIWAPLAGTCGVLLLSLVITLYC | CD8α |
| | | transmembrane |
| | | domain |
| 236 | FWVLVVVGGVLACYSLLVTVAFII | CD28 |
| | FWV | transmembrane |
| | | domain |

### (II)(d)(2)(2e) Intracellular Domain

In certain embodiments, the CAR can comprise an intracellular signaling domain. The various generations of CARs have including an intracellular domain that provides an activating or stimulatory function, such as from CD3ζ, CD3ε, or CD16A. The 2^{nd} and 3^{rd} generation CARs added one or more intracellular domains, respectively, to provide co-stimulatory function, such as from CD28 or 4-1 BB among many others. In certain embodiments, the intracellular signaling domain can comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFRSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4/CD134, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), a ligand that specifically binds with CD83, and a functional variant thereof including the human versions of each of these domains. In some embodiments, the intracellular signaling domain comprises one or more signaling domains selected from a CD3ζ domain, an ITAM, a CD28 domain, 4-1 BB domain, or a functional variant thereof. Table 5 provides amino acid sequences for a few exemplary intracellular signaling domains. 4-1 BB, also known as CD137, transmits a potent costimulatory signal to T cells, promoting differentiation and enhancing long-term survival of T lymphocytes. CD28 is another co-stimulatory molecule on T cells. CD3 zeta (ζ) associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In certain embodiments, as in the case of tisagenlecleucel as described below, the CD3ζ signaling domain of SEQ ID NO: 239 can have a mutation, e.g., a glutamine (Q) to lysine (K) mutation, at amino acid position 14 (see SEQ ID NO: 240).

TABLE 5
Exemplary sequences of
intracellular signaling domains

| SEQ | | |
|---|---|---|
| ID NO: | Sequence | Description |
| 237 | KRGRKKLLYIFKQPFMRPVQTTQEE | 4-1BB signaling |
| | DGCSCRFPEEEEGGCEL | domain |
| 238 | RSKRSRLLHSDYMNMTPRRPGPTR | CD28 signaling |
| | KHYQPYAPPRDFAAYRS | domain |
| 239 | RVKFSRSADAPAYQQGQNQLYNEL | CD3ζ signaling |
| | NLGRREEYDVLDKRRGRDPEMGGK | domain |
| | PRRKNPQEGLYNELQKDKMAEAYS | |
| | EIGMKGERRRGKGHDGLYQGLSTA | |
| | TKDTYDALHMQALPPR | |
| 240 | RVKFSRSADAPAYKQGQNQLYNEL | CD3ζ signaling |
| | NLGRREEYDVLDKRRGRDPEMGGK | domain (with |
| | PRRKNPQEGLYNELQKDKMAEAYS | Q to K mutation |
| | EIGMKGERRRGKGHDGLYQGLSTA | at position 14) |
| | TKDTYDALHMQALPPR | |

### (III)(d)(2)(20 Exemplary CAR Constructs

In certain embodiments, CARs are used to treat a disease or condition associated with a target cell that expresses the antigen targeted by the CAR as described in the uses and methods of treatment disclosed herein. For example, in some embodiments, an anti-CD19 or anti-CD20 or anti-BCMA CAR can be used to target and treat B cell malignancies or B cell-mediated autoimmune conditions or diseases. In other embodiments, an anti-FAP CAR can be used to target and treat solid tumors or fibrosis (e.g., cardiac fibrosis, cancer-associated fibroblasts). Examples of CARs that can be used in accordance with the embodiments described herein include to those disclosed in U.S. Pat. No. 7,446,190 (anti-CD19), U.S. Pat. No. 10,287,350 (anti-CD19), US2021/0363245 (anti-CD19 and anti-CD20), US2023/0167184 (anti-BCMA), US2023/0277589 (anti-BCMA), U.S. Pat. No. 10,543,263 (anti-CD22), U.S. Pat. No. 10,426,797 (anti-CD33), U.S. Pat. No. 10,844,128 (anti-CD123), U.S. Pat. No. 10,428,141 (anti-ROR1), and US2021/0087295 (anti-FAP), each of which is incorporated by reference for all that it teaches about CAR structure and function generically and with respect to the CAR's antigenic specificity and target indications to the extent that it is not inconsistent with this disclosure.

An mRNA disclosed herein encoding a CAR includes both the mature CAR and a signal peptide. In certain embodiments, a mature CAR minimally comprises an antigen binding domain, a transmembrane domain, and an intracellular signaling domain. In some embodiments, a CAR further comprises one or more co-stimulatory domains in the intracellular portion of the CAR. In some embodiments, a CAR further comprises an extracellular hinge or extension domain between the transmembrane domain and the antigen binding domain; this domain can be derived from the same protein as the transmembrane domain. In some embodiments, a CAR can comprise multiple antigen binding domains. In certain embodiments of the mRNA disclosed herein, the CAR is an anti-CD19 CAR, an anti-CD20 CAR, an anti-BCMA CAR, or an anti-FAP CAR.

In certain embodiments comprising an anti-CD19 CAR, the anti-CD19 CAR comprises an anti-CD19 binding domain. Some embodiments of an anti-CD19 CAR comprising an anti-CD19 binding domain further comprise a CD28 hinge, transmembrane, and co-stimulatory domains, and a CD3ζ signaling domain. Some embodiments of an anti-CD19 CAR comprising an anti-CD19 binding domain further comprise a hinge and transmembrane domain from CD8a, a CD28 costimulatory domain, and a CD3 ζ-chain signaling domain. In certain embodiments, an anti-CD19 binding domain comprises a 47G4 scFv. Examples of an anti-CD19 CAR include, without limitation, CAR1 (SEQ ID NO: 4, or with a signal peptide, SEQ ID NO: 1) and CAR2 (SEQ ID NO: 5, or with a signal peptide, SEQ ID NO: 2). In certain embodiments, a CAR-T cell comprising an anti-CD19 CAR comprising CD28 hinge, transmembrane, and co-stimulatory domains exhibits more target cell killing than a CAR-T cell comprising an anti-CD19 CAR comprising CD8α hinge and transmembrane domains, and a CD28 co-stimulatory domain.

Examples of anti-CD19 CARs include those incorporating a CD19 binding moiety derived from the mouse antibody FMC63. FMC63 and the derived scFv have been described in Nicholson et al., 1997, Mol. Immun. 34(16-17):1157-1165 and PCT Application Publication Nos. WO 2018/213337 and WO 2015/187528, the entire contents of each of which are incorporated by reference herein for all that they teach about anti-CD19 CARs and their use.

TABLE 6
Exemplary sequences of
anti-CD19 scFv and components

| SEQ | | |
|---|---|---|
| ID NO: | Amino Acid Sequence | Description |
| 241 | DIQMTQTTSSLSASLGDRVTISCRA | Anti-CD19 FMC63 scFv |
| | SQDISKYLNWYQQKPDGTVKLLIYH | entire sequence, with |
| | TSRLHSGVPSRFSGSGSGTDYSLTI | Whitlow linker |
| | SNLEQEDIATYFCQQGNTLPYTFGG | |
| | GTKLEITGSTSGSGKPGSGEGSTK | |
| | GEVKLQESGPGLVAPSQSLSVTCT | |
| | VSGVSLPDYGVSWIRQPPRKGLEW | |
| | LGVIWGSETTYYNSALKSRLTIIKDN | |
| | SKSQVFLKMNSLQTDDTAIYYCAKH | |
| | YYYGGSYAMDYWGQGTSVTVSS | |
| 242 | DIQMTQTTSSLSASLGDRVTISCRA | Anti-CD19 FMC63 scFv |
| | SQDISKYLNWYQQKPDGTVKLLIYH | light chain variable |
| | TSRLHSGVPSRFSGSGSGTDYSLTI | region |
| | SNLEQEDIATYFCQQGNTLPYTFGG | |
| | GTKLEIT | |
| 243 | QDISKY | Anti-CD19 FMC63 scFv |
| | | light chain CDR1 |
| 244 | HTS | Anti-CD19 FMC63 scFv |
| | | light chain CDR2 |
| 245 | QQGNTLPYT | Anti-CD19 FMC63 scFv |
| | | light chain CDR3 |
| 246 | GSTSGSGKPGSGEGSTKG | Whitlow linker |
| 247 | EVKLQESGPGLVAPSQSLSVTCTV | Anti-CD19 FMC63 scFv |
| | SGVSLPDYGVSWIRQPPRKGLEWL | heavy chain variable |
| | GVIWGSETTYYNSALKSRLTIIKDNS | region |
| | KSQVFLKMNSLQTDDTAIYYCAKHY | |
| | YYGGSYAMDYWGQGTSVTVSS | |
| 248 | GVSLPDYG | Anti-CD19 FMC63 scFv |
| | | heavy chain CDR1 |
| 249 | IWGSETT | Anti-CD19 FMC63 scFv |
| | | heavy chain CDR2 |
| 250 | AKHYYYGGSYAMDY | Anti-CD19 FMC63 scFv |
| | | heavy chain CDR3 |
| 251 | DIQMTQTTSSLSASLGDRVTISCRA | Anti-CD19 FMC63 scFv |
| | SQDISKYLNWYQQKPDGTVKLLIYH | entire sequence, with |
| | TSRLHSGVPSRFSGSGSGTDYSLTI | 3xG₄S linker |
| | SNLEQEDIATYFCQQGNTLPYTFGG | |
| | GTKLEITGGGGSGGGGSGGGGSE | |
| | VKLQESGPGLVAPSQSLSVTCTVS | |
| | GVSLPDYGVSWIRQPPRKGLEWLG | |
| | VIWGSETTYYNSALKSRLTIIKDNSK | |
| | SQVFLKMNSLQTDDTAIYYCAKHYY | |
| | YGGSYAMDYWGQGTSVTVSS | |
| 252 | GGGGSGGGGSGGGGS | 3xG₄S linker |

In some instances, the anti-CD19 CAR is the CAR found in tisagenlecleucel (Vairy et al., 2018, Drug Des Devel Ther. 12: 3885-3898), lisocabtagene maraleucel, or axicabtagene ciloleucel and brexucabtagene autoleucel (Cappell et al., 2023, Nat Rev Clin Oncol 20: 359-371) which use the same CAR. The entire contents of each of foregoing references in this paragraph are incorporated by reference for all that they teach about the design, structure, and activity of anti-CD19 CARs.

TABLE 7
Exemplary sequences of CD19 CARs*

| SEQ | | |
|---|---|---|
| ID NO: | Sequence | Description |
| 253 | atggccttaccagtgaccgccttgctcctgccgctggccttg | Tisagenlecleucel |
| | ctgctccacgccgccaggccggacatccagatgacacag | CD19 CAR |
| | actacatcctccctgtctgcctctctgggagacagagtcacc | nucleotide |
| | atcagttgcagggcaagtcaggacattagtaaatatttaaa | sequence |
| | ttggtatcagcagaaaccagatggaactgttaaactcctga | |
| | tctaccatacatcaagattacactcaggagtcccatcaagg | |
| | ttcagtggcagtgggtctggaacagattattctctcaccatta | |
| | gcaacctggagcaagaagatattgccacttacttttgccaa | |
| | cagggtaatacgcttccgtacacgttcggaggggggacc | |
| | aagctggagatcacaggtggcggtggctcgggcggtggt | |
| | gggtcgggtggcggcggatctgaggtgaaactgcaggag | |
| | tcaggacctggcctggtggcgccctcacagagcctgtccg | |
| | tcacatgcactgtctcaggggtctcattacccgactatggtgt | |
| | aagctggattcgccagcctccacgaaagggtctggagtg | |
| | gctgggagtaatatggggtagtgaaaccacatactataatt | |
| | cagctctcaaatccagactgaccatcatcaaggacaactc | |
| | caagagccaagttttcttaaaaatgaacagtctgcaaactg | |
| | atgacacagccatttactactgtgccaaacattattactacg | |
| | gtggtagctatgctatggactactggggccaaggaacctc | |
| | agtcaccgtctcctcaaccacgacgccagcgccgcgacc | |
| | accaacaccggcgcccaccatcgcgtcgcagcccctgtc | |
| | cctgcgcccagaggcgtgccggccagcggcggggggc | |
| | gcagtgcacacgagggggctggacttcgcctgtgatatct | |
| | acatctgggcgcccttggccgggacttgtggggtccttctcc | |
| | tgtcactggttatcaccctttactgcaaacggggcagaaag | |
| | aaactcctgtatatattcaaacaaccatttatgagaccagta | |
| | caaactactcaagaggaagatggctgtagctgccgatttc | |
| | cagaagaagaagaaggaggatgtgaactgagagtgaa | |
| | gttcagcaggagcgcagacgcccccgcgtacaagcagg | |
| | gccagaaccagctctataacgagctcaatctaggacgaa | |
| | gagaggagtacgatgttttggacaagagacgtggccggg | |
| | accctgagatggggggaaagccgagaaggaagaaccc | |
| | tcaggaaggcctgtacaatgaactgcagaaagataagat | |
| | ggcggaggcctacagtgagattgggatgaaaggcgagc | |
| | gccggaggggcaaggggcacgatggcctttaccagggt | |
| | ctcagtacagccaccaaggacacctacgacgcccttcac | |
| | atgcaggccctgccccctcgc | |
| 254 | MALPVTALLLPLALLLHAARPDIQMTQTTS | Tisagenlecleucel |
| | SLSASLGDRVTISCRASQDISKYLNWYQQ | CD19 CAR |
| | KPDGTVKLLIYHTSRLHSGVPSRFSGSGS | amino acid |
| | GTDYSLTISNLEQEDIATYFCQQGNTLPYT | sequence |
| | FGGGTKLEITGGGGSGGGGSGGGGSEVK | |
| | LQESGPGLVAPSQSLSVTCTVSGVSLPDY | |
| | GVSWIRQPPRKGLEWLGVIWGSETTYYN | |
| | SALKSRLTIIKDNSKSQVFLKMNSLQTDDT | |
| | AIYYCAKHYYYGGSYAMDYWGQGTSVTV | |
| | SSTTTPAPRPPTPAPTIASQPLSLRPEACR | |
| | PAAGGAVHTRGLDFACDIYIWAPLAGTCG | |
| | VLLLSLVITLYCKRGRKKLLYIFKQPFMRPV | |
| | QTTQEEDGCSCRFPEEEEGGCELRVKFS | |
| | RSADAPAYKQGQNQLYNELNLGRREEYD | |
| | VLDKRRGRDPEMGGKPRRKNPQEGLYNE | |
| | LQKDKMAEAYSEIGMKGERRRGKGHDGL | |
| | YQGLSTATKDTYDALHMQALPPR | |
| 255 | atgctgctgctggtgaccagcctgctgctgtgcgagctgcc | Lisocabtagene |
| | ccaccccgcctttctgctgatccccgacatccagatgaccc | maraleucel CD19 |
| | agaccacctccagcctgagcgccagcctgggcgaccgg | CAR nucleotide |
| | gtgaccatcagctgccgggccagccaggacatcagcaa | sequence |
| | gtacctgaactggtatcagcagaagcccgacggcaccgt | |
| | caagctgctgatctaccacaccagccggctgcacagcgg | |
| | cgtgcccagccggtttagcggcagcggctccggcaccga | |
| | ctacagcctgaccatctccaacctggaacaggaagatatc | |
| | gccacctacttttgccagcagggcaacacactgccctaca | |
| | cctttggcggcggaacaaagctggaaatcaccggcagca | |
| | cctccggcagcggcaagcctggcagcggcgagggcag | |
| | caccaagggcgaggtgaagctgcaggaaagcggccct | |
| | ggcctggtggcccccagccagagcctgagcgtgacctgc | |
| | accgtgagcggcgtgagcctgcccgactacggcgtgagc | |
| | tggatccggcagccccccaggaagggcctggaatggctg | |
| | ggcgtgatctggggcagcgagaccacctactacaacagc | |
| | gccctgaagagccggctgaccatcatcaaggacaacag | |
| | caagagccaggtgttcctgaagatgaacagcctgcagac | |
| | cgacgacaccgccatctactactgcgccaagcactactac | |
| | tacggcggcagctacgccatggactactggggccagggc | |
| | accagcgtgaccgtgagcagcgaatctaagtacggaccg | |
| | ccctgccccccttgccctatgttctgggtgctggtggtggtcg | |
| | gaggcgtgctggcctgctacagcctgctggtcaccgtggc | |
| | cttcatcatcttttgggtgaaacggggcagaaagaaactcc | |
| | tgtatatattcaaacaaccatttatgagaccagtacaaacta | |
| | ctcaagaggaagatggctgtagctgccgatttccagaaga | |
| | agaagaaggaggatgtgaactgcgggtgaagttcagca | |
| | gaagcgccgacgcccctgcctaccagcagggccagaat | |
| | cagctgtacaacgagctgaacctgggcagaagggaaga | |
| | gtacgacgtcctggataagcggagaggccgggaccctg | |
| | agatgggcggcaagcctcggcggaagaacccccagga | |
| | aggcctgtataacgaactgcagaaagacaagatggccg | |
| | aggcctacagcgagatcggcatgaagggcgagcggag | |
| | gcggggcaagggccacgacggcctgtatcagggcctgtc | |
| | caccgccaccaaggatacctacgacgccctgcacatgca | |
| | ggccctgcccccaagg | |
| 256 | MLLLVTSLLLCELPHPAFLLIPDIQMTQTTS | Lisocabtagene |
| | SLSASLGDRVTISCRASQDISKYLNWYQQ | maraleucel CD19 |
| | KPDGTVKLLIYHTSRLHSGVPSRFSGSGS | CAR amino acid |
| | GTDYSLTISNLEQEDIATYFCQQGNTLPYT | sequence |
| | FGGGTKLEITGSTSGSGKPGSGEGSTKGE | |
| | VKLQESGPGLVAPSQSLSVTCTVSGVSLP | |
| | DYGVSWIRQPPRKGLEWLGVIWGSETTY | |
| | YNSALKSRLTIIKDNSKSQVFLKMNSLQTD | |
| | DTAIYYCAKHYYYGGSYAMDYWGQGTSV | |
| | TVSSESKYGPPCPPCPMFWVLVVVGGVL | |
| | ACYSLLVTVAFIIFWVKRGRKKLLYIFKQPF | |
| | MRPVQTTQEEDGCSCRFPEEEEGGCELR | |
| | VKFSRSADAPAYQQGQNQLYNELNLGRR | |
| | EEYDVLDKRRGRDPEMGGKPRRKNPQE | |
| | GLYNELQKDKMAEAYSEIGMKGERRRGK | |
| | GHDGLYQGLSTATKDTYDALHMQALPPR | |
| 257 | atgcttctcctggtgacaagccttctgctctgtgagttaccac | Axicabtagene |
| | acccagcattcctcctgatcccagacatccagatgacaca | ciloleucel CD19 |
| | gactacatcctccctgtctgcctctctgggagacagagtca | CAR nucleotide |
| | ccatcagttgcagggcaagtcaggacattagtaaatattta | sequence |
| | aattggtatcagcagaaaccagatggaactgttaaactcct | |
| | gatctaccatacatcaagattacactcaggagtcccatcaa | |
| | ggttcagtggcagtgggtctggaacagattattctctcacca | |
| | ttagcaacctggagcaagaagatattgccacttacttttgcc | |
| | aacagggtaatacgcttccgtacacgttcggagggggga | |
| | ctaagttggaaataacaggctccacctctggatccggcaa | |
| | gcccggatctggcgagggatccaccaagggcgaggtga | |
| | aactgcaggagtcaggacctggcctggtggcgccctcac | |
| | agagcctgtccgtcacatgcactgtctcaggggtctcattac | |
| | ccgactatggtgtaagctggattcgccagcctccacgaaa | |
| | gggtctggagtggctgggagtaatatggggtagtgaaacc | |
| | acatactataattcagctctcaaatccagactgaccatcatc | |
| | aaggacaactccaagagccaagttttcttaaaaatgaaca | |
| | gtctgcaaactgatgacacagccatttactactgtgccaaa | |
| | cattattactacggtggtagctatgctatggactactggggtc | |
| | aaggaacctcagtcaccgtctcctcagcggccgcaattga | |
| | agttatgtatcctcctccttacctagacaatgagaagagca | |
| | atggaaccattatccatgtgaaagggaaacacctttgtcca | |
| | agtcccctatttcccggaccttctaagcccttttgggtgctggt | |
| | ggtggttgggggagtcctggcttgctatagcttgctagtaac | |
| | agtggcctttattattttctgggtgaggagtaagaggagcag | |
| | gctcctgcacagtgactacatgaacatgactccccgccgc | |
| | cccgggcccacccgcaagcattaccagccctatgcccca | |
| | ccacgcgacttcgcagcctatcgctccagagtgaagttca | |
| | gcaggagcgcagacgcccccgcgtaccagcagggcca | |
| | gaaccagctctataacgagctcaatctaggacgaagaga | |
| | ggagtacgatgttttggacaagagacgtggccgggaccct | |
| | gagatggggggaaagccgagaaggaagaaccctcagg | |
| | aaggcctgtacaatgaactgcagaaagataagatggcgg | |
| | aggcctacagtgagattgggatgaaaggcgagcgccgg | |
| | aggggcaaggggcacgatggcctttaccagggtctcagt | |
| | acagccaccaaggacacctacgacgcccttcacatgcag | |
| | gccctgccccctcgc | |
| 258 | MLLLVTSLLLCELPHPAFLLIPDIQMTQTTS | Axicabtagene |
| | SLSASLGDRVTISCRASQDISKYLNWYQQ | ciloleucel CD19 |
| | KPDGTVKLLIYHTSRLHSGVPSRFSGSGS | CAR amino acid |
| | GTDYSLTISNLEQEDIATYFCQQGNTLPYT | sequence |
| | FGGGTKLEITGSTSGSGKPGSGEGSTKGE | |
| | VKLQESGPGLVAPSQSLSVTCTVSGVSLP | |
| | DYGVSWIRQPPRKGLEWLGVIWGSETTY | |
| | YNSALKSRLTIIKDNSKSQVFLKMNSLQTD | |
| | DTAIYYCAKHYYYGGSYAMDYWGQGTSV | |
| | TVSSAAAIEVMYPPPYLDNEKSNGTIIHVK | |
| | GKHLCPSPLFPGPSKPFWVLVVVGGVLAC | |
| | YSLLVTVAFIIFWVRSKRSRLLHSDYMNMT | |
| | PRRPGPTRKHYQPYAPPRDFAAYRSRVK | |
| | FSRSADAPAYQQGQNQLYNELNLGRREE | |
| | YDVLDKRRGRDPEMGGKPRRKNPQEGLY | |
| | NELQKDKMAEAYSEIGMKGERRRGKGHD | |
| | GLYQGLSTATKDTYDALHMQALPPR | |

*Nucleotide sequences are presented here (and throughout) as DNA sequences; t should be understood as u for corresponding RNA sequences.

TABLE 8
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB signaling domain | 997-1122 | 333-374 |
| CD3ζ signaling domain | 1123-1458 | 375-486 |

TABLE 9
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB signaling domain | 922-1047 | 308-349 |
| CD3ζ signaling domain | 1048-1383 | 350-461 |

TABLE 10
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 signaling domain | 1009-1131 | 337-377 |
| CD3ζ signaling domain | 1132-1467 | 378-489 |

CAR based on 47G4 are disclosed in U.S. Pat. No. 10,287,350 which is incorporated by reference herein for all that it teaches about anti-CD19 CARs and their use. In some embodiments, the extracellular binding domain of the CD19 CAR is derived from an antibody specific to CD19, including, for example, SJ25C1 (Bejcek et al., 1995, Cancer Res. 55:2346-2351), HD37 (Pezutto et al., 1987, J. Immunol. 138(9):2793-2799), 4G7 (Meeker et al., 1984, Hybridoma 3:305-320), B43 (Bejcek et al., 1995 Cancer Res 55(11):2346-2351), BLY3 (Bejcek et al., 1995 Cancer Res 55(11):2346-2351), B4 (Freedman et al., 1987, Blood 70:418-427), B4 HB12b (Kansas & Tedder, 1991, J. Immunol. 147:4094-4102; Yazawa et al., 2005, Proc. Natl. Acad. Sci. USA 102:15178-15183; Herbst et al., J. Pharmacol. Exp. Ther. 335:213-222 (2010)), BU12 (Callard et al., 1992, J. Immunology, 148(10): 2983-2987), and CLB-CD19 (De Rie, 1989, Cell. Immunol. 118:368-381). In any of these embodiments, the extracellular binding domain of the CD19 CAR can comprise the V_{H}, the V_{L}, and/or one or more CDRs of any of the antibodies.

TABLE 11
Exemplary sequences of 47G4-based anti-CD19 CAR

| SEQ | | |
|---|---|---|
| ID | | |
| NO: | Sequence | Description |
| 107 | MALPVTALLLPLALLLHAARP | CD8 signal |
| | | peptide |
| 259 | EIVLTQSPGTLSLSPGERATLSCRASQSVS | CD19 antibody |
| | SSYLAWYQQKPGQAPRLLIYGASSRATGI | (47G4 scFv) |
| | PDRFSGSGSGTDFTLTISRLEPEDFAVYY | |
| | CQQYGSSRFTFGPGTKVDIKGSTSGSGK | |
| | PGSGEGSTKGQVQLVQSGAEVKKPGSSV | |
| | KVSCKDSGGTFSSYAISWVRQAPGQGLE | |
| | WMGGIIPIFGTTNYAQQFQGRVTITADEST | |
| | STAYMELSSLRSEDTAVYYCAREAVAAD | |
| | WLDPWGQGTLVTVSS | |
| 231 | IEVMYPPPYLDNEKSNGTIIHVKGKHLCPS | CD28 hinge |
| | PLFPGPSKP | |
| 236 | FWVLVVVGGVLACYSLLVTVAFIIFWV | CD28 |
| | | transmembrane |
| | | domain |
| 238 | RSKRSRLLHSDYMNMTPRRPGPTRKHYQ | CD28 |
| | PYAPPRDFAAYRS | cytoplasmic |
| | | (co-stim) |
| 239 | RVKFSRSADAPAYQQGQNQLYNELNLGR | CD3ζ (stim) |
| | REEYDVLDKRRGRDPEMGGKPRRKNPQ | |
| | EGLYNELQKDKMAEAYSEIGMKGERRRG | |
| | KGHDGLYQGLSTATKDTYDALHMQALPP | |
| | R | |
| 260 | FVPVFLPAKPTTTPAPRPPTPAPTIASQPL | CD8 hinge |
| | SLRPEACRPAAGGAVHTRGLDFACD | |
| 261 | IYIWAPLAGTCGVLLLSLVITLYCNHRN | CD8 |
| | | transmembrane |
| | | domain |

CD20 is an antigen found on the surface of B cells as early as the pro-B phase and progressively at increasing levels until B cell maturity, as well as on the cells of most B-cell neoplasms. CD20 positive cells are also sometimes found in cases of Hodgkin's disease, myeloma, and thymoma. Examples of anti-CD20 CARs include those incorporating a CD20 binding moiety derived from an antibody specific to CD20, including, for example, MB-106 (Fred Hutchinson Cancer Research Center, see Shadman et al., 2019, Blood 134(Suppl. 1):3235), UCART20 (Cellectis, www.cellbiomedgroup.com), or C-CAR066 (Cellular Biomedicine Group, see Liang et al., 2021, J. Clin. Oncol. 39(15) suppl:2508). In some embodiments, the extracellular binding domain of the anti-CD20 CAR is derived from an antibody specific to CD20, including, for example, Leu16, 2.1.2, IF5, 1.5.3, rituximab, obinutuzumab, ibritumomab, ofatumumab, tositumumab, odronextamab, veltuzumab, ublituximab, and ocrelizumab. In some embodiments, the extracellular binding domain of the anti-CD20 CAR comprises an scFv derived from the Leu16 monoclonal antibody, which comprises the heavy chain variable region (V_{H}) and the light chain variable region (V_{L}) of Leu16 connected by a linker (See Wu et al., 2001, Protein Engineering. 14(12):1025-1033), such as CAR22 and CAR25 described herein. In some embodiments, the extracellular binding domain of the anti-CD20 CAR comprises an scFv derived from the monoclonal antibody, 2.1.2 (WO2006130458A2), which comprises the heavy chain variable region (V_{H}) and the light chain variable region (V_{L}) of 2.1.2 connected by a linker, such as CAR7 described herein. The entire contents of each of foregoing references in this paragraph are incorporated by reference for all that they teach about the design, structure, and activity of anti-CD20 CARs.

In certain embodiments comprising an anti-CD20 CAR, the anti-CD20 CAR comprises a Leu16 scFv. In some embodiments, the anti-CD20 CAR comprising a Leu16 scFv further comprises an IgG4 hinge, CD28 transmembrane domain, 4-1 BB costimulation, and a CD3ζ signaling domain. Examples of such an anti-CD20 CAR include, without limitation, CAR25 (SEQ ID NO: 6, or with a signal peptide, SEQ ID NO: 3). In some embodiments, the anti-CD20 CAR comprising a Leu16 scFv further comprises an IgG4 hinge, CD28 transmembrane and costimulation domains, 4-1BB costimulation, and a CD3ζ signaling domain. Examples of such an anti-CD20 CAR include CAR22 (SEQ ID NO: 77, or with a signal peptide, SEQ ID NO: 75).

TABLE 12
Exemplary sequences of anti-CD20 scFv and components

| SEQ ID NO: | Amino Acid Sequence | Description |
|---|---|---|
| 262 | DIVLTQSPAILSASPGEKVTMTCRA | Anti-CD20 Leu16 scFv |
| | SSSVNYMDWYQKKPGSSPKPWIYA | entire sequence, with |
| | TSNLASGVPARFSGSGSGTSYSLTI | Whitlow linker |
| | SRVEAEDAATYYCQQWSFNPPTFG | |
| | GGTKLEIKGSTSGSGKPGSGEGST | |
| | KGEVQLQQSGAELVKPGASVKMSC | |
| | KASGYTFTSYNMHWVKQTPGQGL | |
| | EWIGAIYPGNGDTSYNQKFKGKATL | |
| | TADKSSSTAYMQLSSLTSEDSADY | |
| | YCARSNYYGSSYWFFDVWGAGTT | |
| | VTVSS | |
| 263 | DIVLTQSPAILSASPGEKVTMTCRA | Anti-CD20 Leu16 scFv |
| | SSSVNYMDWYQKKPGSSPKPWIYA | light chain variable |
| | TSNLASGVPARFSGSGSGTSYSLTI | region |
| | SRVEAEDAATYYCQQWSFNPPTFG | |
| | GGTKLEIK | |
| 264 | RASSSVNYMD | Anti-CD20 Leu16 scFv |
| | | light chain CDR1 |
| 265 | ATSNLAS | Anti-CD20 Leu16 scFv |
| | | light chain CDR2 |
| 267 | QQWSFNPPT | Anti-CD20 Leu16 scFv |
| | | light chain CDR3 |
| 268 | EVQLQQSGAELVKPGASVKMSCKA | Anti-CD20 Leu16 scFv |
| | SGYTFTSYNMHWVKQTPGQGLEWI | heavy chain |
| | GAIYPGNGDTSYNQKFKGKATLTA | |
| | DKSSSTAYMQLSSLTSEDSADYYC | |
| | ARSNYYGSSYWFFDVWGAGTTVT | |
| | VSS | |
| 269 | SYNMH | Anti-CD20 Leu16 scFv |
| | | heavy chain CDR1 |
| 270 | AIYPGNGDTSYNQKFKG | Anti-CD20 Leu16 scFv |
| | | heavy chain CDR2 |
| 266 | SNYYGSSYWFFDV | Anti-CD20 Leu16 scFv |
| | | heavy chain CDR3 |
| 87 | DIVLTQSPAILSASPGEKVTMTCRA | Anti-CD20 Leu16 scFv |
| | SSSVNYMDWYQKKPGSSPKPWIYA | entire sequence, with |
| | TSNLASGVPARFSGSGSGTSYSLTI | (G₃S)₂-(G₄S) linker |
| | SRVEAEDAATYYCQQWSFNPPTFG | |
| | GGTKLEIKGSTSGGGSGGGSGGG | |
| | GSSEVQLQQSGAELVKPGASVKMS | |
| | CKASGYTFTSYNMHWVKQTPGQG | |
| | LEWIGAIYPGNGDTSYNQKFKGKAT | |
| | LTADKSSSTAYMQLSSLTSEDSADY | |
| | YCARSNYYGSSYWFFDVWGAGTT | |
| | VTVSS | |
| 229 | GSTSGGGSGGGSGGGGSS | (G₃S)₂-(G₄S) linker |
| 88 | DIVMTQTPHSSPVTLGQPASISCRS | Anti-CD20 2.1.2 scFv |
| | SQSLVSRDGNTYLSWLQQRPGQP | entire sequence, with |
| | PRLLIYKISNRFSGVPNRFSGSGAG | (G₄S)₃ linker |
| | TDFTLKISRVKAEDVGVYYCMQATQ | |
| | FPLTFGQGTRLEIKGGGGSGGGGS | |
| | GGGGSEVQLVQSGAEVKKPGESLK | |
| | ISCKGSGYSFTSYWIGWVRQMPGK | |
| | GLEWMGIIYPGDSDTRYSPSFQGQ | |
| | VTISADKSISTAYLQWSSLKASDTA | |
| | MYYCARQGDFWSGYGGMDVWGQ | |
| | GTTVTVSS | |
| 331 | DIVMTQTPHSSPVTLGQPASISCRS | Anti-CD20 2.1.2 scFv |
| | SQSLVSRDGNTYLSWLQQRPGQP | light chain variable |
| | PRLLIYKISNRFSGVPNRFSGSGAG | region |
| | TDFTLKISRVKAEDVGVYYCMQATQ | |
| | FPLTFGQGTRLEIK | |
| 332 | RSSQSLVSRDGNTYLS | Anti-CD20 2.1.2 scFv |
| | | light chain CDR1 |
| 333 | KISNRFS | Anti-CD20 2.1.2 scFv |
| | | light chain CDR2 |
| 334 | MQATQFPLT | Anti-CD20 2.1.2 scFv |
| | | light chain CDR3 |
| 252 | GGGGSGGGGSGGGGS | (G₄S)₃ or 3x G₄S linker |
| 335 | EVQLVQSGAEVKKPGESLKISCKGS | Anti-CD20 2.1.2 scFv |
| | GYSFTSYWIGWVRQMPGKGLEWM | heavy chain variable |
| | GIIYPGDSDTRYSPSFQGQVTISAD | region |
| | KSISTAYLQWSSLKASDTAMYYCAR | |
| | QGDFWSGYGGMDVWGQGTTVTV | |
| | SS | |
| | SYWIG | Anti-CD20 2.1.2 scFv |
| | | heavy chain CDR1 |
| 336 | IIYPGDSDTRYSPSFQG | Anti-CD20 2.1.2 scFv |
| | | heavy chain CDR2 |
| 337 | QGDFWSGYGGMDV | Anti-CD20 2.1.2 scFv |
| | | heavy chain CDR3 |

In certain embodiments comprising an anti-CD20 CAR, the anti-CD20 CAR comprises a 2.1.2 scFv. In some embodiments, the anti-CD20 CAR comprising a 2.1.2 scFv further comprises CD28 hinge, transmembrane, and costimulation domains and a CD3ζ signaling domain. Examples of such an anti-CD20 CAR include, without limitation, CAR7 (SEQ ID NO: 76, or with a signal peptide, SEQ ID NO: 74).

In certain embodiments, the improved mRNA encodes an anti-BCMA chimeric antigen receptor (CAR). BCMA is a tumor necrosis family receptor (TNFR) member expressed on cells of the B cell lineage, with the highest expression on terminally differentiated B cells or mature B lymphocytes. BCMA is involved in mediating survival of plasma cells for maintaining long-term humoral immunity. Expression of BCMA has been recently linked to a number of cancers, such as multiple myeloma, Hodgkin's and non-Hodgkin's lymphoma, various leukemias, and glioblastoma. Examples of anti-BCMA CARs include those incorporating a BCMA binding moiety derived from C11D5.3, a mouse monoclonal antibody as described in Carpenter et al., 2013, Clin. Cancer Res. 19(8):2048-2060. See also PCT Application Publication No. WO 2010/104949. In some embodiments, the extracellular binding domain of the BCMA CAR comprises a scFv derived from another mouse monoclonal antibody, C12A3.2, as described in Carpenter et al., 2013, Clin. Cancer Res. 19(8):2048-2060 and PCT Application Publication No. WO2010104949. In some embodiments, the extracellular binding domain of the BCMA CAR comprises an scFv derived from a mouse monoclonal antibody with high specificity to human BCMA, referred to as BB2121 in Friedman et al., 2018, Hum. Gene Ther. 29(5):585-601. See also, PCT Application Publication No. WO2012163805. In some embodiments, the extracellular binding domain of the BCMA CAR comprises single variable fragments of two heavy chains (VHH) that can bind to two epitopes of BCMA as described in Zhao et al., 2018, J. Hematol. Oncol. 11(1):141, also referred to as LCAR-B38M. See also, PCT Application Publication No. WO 2018/028647. In some embodiments, the extracellular binding domain of the BCMA CAR comprises a fully human heavy-chain variable domain (FHVH) as described in Lam et al., 2020, Nat. Commun. 11(1):283, also referred to as FHVH33. See also, PCT Application Publication No. WO 2019/006072. In some embodiments, the extracellular binding domain of the BCMA CAR comprises a scFv derived from CT103A (or CAR0085) as described in U.S. Pat. No. 11,026,975 B2. Further anti-BCMA CARs are disclosed in U.S. Patent Application Publication Nos. 2020/0246381 and 2020/0339699. The entire contents of each of foregoing references in this paragraph are incorporated by reference for all that they teach about the design, structure, and activity of anti-BCMA CARs.

TABLE 13
Exemplary sequences of anti-BCMA binder and components

| SEQ ID NO: | Amino Acid Sequence | Description |
|---|---|---|
| 271 | DIVLTQSPASLAMSLGKRATISCRA | Anti-BCMA C11D5.3 |
| | SESVSVIGAHLIHWYQQKPGQPPKL | scFv entire sequence, |
| | LIYLASNLETGVPARFSGSGSGTDF | with Whitlow linker |
| | TLTIDPVEEDDVAIYSCLQSRIFPR | |
| | TFGGGTKLEIKGSTSGSGKPGSGEG | |
| | STKGQIQLVQSGPELKKPGETVKIS | |
| | CKASGYTFTDYSINWVKRAPGKGLK | |
| | WMGWINTETREPAYAYDFRGRFAFS | |
| | LETSASTAYLQINNLKYEDTATYFC | |
| | ALDYSYAMDYWGQGTSVTVSS | |
| 272 | DIVLTQSPASLAMSLGKRATISCRA | Anti-BCMA C11D5.3 |
| | SESVSVIGAHLIHWYQQKPGQPPKL | scFv light chain |
| | LIYLASNLETGVPARFSGSGSGTDF | variable region |
| | TLTIDPVEEDDVAIYSCLQSRIFPR | |
| | TFGGGTKLEIK | |
| 273 | RASESVSVIGAHLIH | Anti-BCMA C11D5.3 |
| | | scFv light chain CDR1 |
| 274 | LASNLET | Anti-BCMA C11D5.3 |
| | | scFv light chain CDR2 |
| 275 | LQSRIFPRT | Anti-BCMA C11D5.3 |
| | | scFv light chain CDR3 |
| 276 | QIQLVQSGPELKKPGETVKISCKAS | Anti-BCMA C11D5.3 |
| | GYTFTDYSINWVKRAPGKGLKWMGW | scFv heavy chain |
| | INTETREPAYAYDFRGRFAFSLETS | variable region |
| | ASTAYLQINNLKYEDTATYFCALDY | |
| | SYAMDYWGQGTSVTVSS | |
| 277 | DYSIN | Anti-BCMA C11D5.3 |
| | | scFv heavy chain |
| | | CDR1 |
| 278 | WINTETREPAYAYDFRG | Anti-BCMA C11D5.3 |
| | | scFv heavy chain |
| | | CDR2 |
| 279 | DYSYAMDY | Anti-BCMA C11D5.3 |
| | | scFv heavy chain |
| | | CDR3 |
| 280 | DIVLTQSPPSLAMSLGKRATISCRA | Anti-BCMA C12A3.2 |
| | SESVTILGSHLIYWYQQKPGQPPTL | scFv entire sequence, |
| | LIQLASNVQTGVPARFSGSGSRTDF | with Whitlow linker |
| | TLTIDPVEEDDVAVYYCLQSRTIPR | |
| | TFGGGTKLEIKGSTSGSGKPGSGEG | |
| | STKGQIQLVQSGPELKKPGETVKIS | |
| | CKASGYTFRHYSMNWVKQAPGKGLK | |
| | WMGRINTESGVPIYADDFKGRFAFS | |
| | VETSASTAYLVINNLKDEDTASYFC | |
| | SNDYLYSLDFWGQGTALTVSS | |
| 281 | DIVLTQSPPSLAMSLGKRATISCRA | Anti-BCMA C12A3.2 |
| | SESVTILGSHLIYWYQQKPGQPPTL | scFv light chain |
| | LIQLASNVQTGVPARFSGSGSRTDF | variable region |
| | TLTIDPVEEDDVAVYYCLQSRTIPR | |
| | TFGGGTKLEIK | |
| 282 | RASESVTILGSHLIY | Anti-BCMA C12A3.2 |
| | | scFv light chain CDR1 |
| 283 | LASNVQT | Anti-BCMA C12A3.2 |
| | | scFv light chain CDR2 |
| 284 | LQSRTIPRT | Anti-BCMA C12A3.2 |
| | | scFv light chain CDR3 |
| 285 | QIQLVQSGPELKKPGETVKISCKAS | Anti-BCMA C12A3.2 |
| | GYTFRHYSMNWVKQAPGKGLKWMGR | scFv heavy chain |
| | INTESGVPIYADDFKGRFAFSVETS | variable region |
| | ASTAYLVINNLKDEDTASYFCSNDY | |
| | LYSLDFWGQGTALTVSS | |
| 286 | HYSMN | Anti-BCMA C12A3.2 |
| | | scFv heavy chain |
| | | CDR1 |
| 287 | RINTESGVPIYADDFKG | Anti-BCMA C12A3.2 |
| | | scFv heavy chain |
| | | CDR2 |
| 288 | DYLYSLDF | Anti-BCMA C12A3.2 |
| | | scFv heavy chain |
| | | CDR3 |
| 289 | EVQLLESGGGLVQPGGSLRLSCAAS | Anti-BCMA FHVH33 |
| | GFTFSSYAMSWVRQAPGKGLEWVSS | entire sequence |
| | ISGSGDYIYYADSVKGRFTISRDIS | |
| | KNTLYLQMNSLRAEDTAVYYCAKEG | |
| | TGANSSLADYRGQGTLVTVSS | |
| 290 | GFTFSSYA | Anti-BCMA FHVH33 |
| | | CDR1 |
| 291 | ISGSGDYI | Anti-BCMA FHVH33 |
| | | CDR2 |
| 292 | AKEGTGANSSLADY | Anti-BCMA FHVH33 |
| | | CDR3 |
| 293 | DIQMTQSPSSLSASVGDRVTITCRA | Anti-BCMA CT103A |
| | SQSISSYLNWYQQKPGKAPKLLIYA | scFv entire sequence, |
| | ASSLQSGVPSRFSGSGSGTDFTLTI | with Whitlow linker |
| | SSLQPEDFATYYCQQKYDLLTFGGG | |
| | TKVEIKGSTSGSGKPGSGEGSTKGQ | |
| | LQLQESGPGLVKPSETLSLTCTVSG | |
| | GSISSSSYYWGWIRQPPGKGLEWIG | |
| | SISYSGSTYYNPSLKSRVTISVDTS | |
| | KNQFSLKLSSVTAADTAVYYCARDR | |
| | GDTILDVWGQGTMVTVSS | |
| 294 | DIQMTQSPSSLSASVGDRVTITCRA | Anti-BCMA CT103A |
| | SQSISSYLNWYQQKPGKAPKLLIYA | scFv light chain |
| | ASSLQSGVPSRFSGSGSGTDFTLTI | variable region |
| | SSLQPEDFATYYCQQKYDLLTFGGG | |
| | TKVEIK | |
| 295 | QSISSY | Anti-BCMA CT103A |
| | | scFv light chain CDR1 |
| 296 | AAS | Anti-BCMA CT103A |
| | | scFv light chain CDR2 |
| 297 | QQKYDLLT | Anti-BCMA CT103A |
| | | scFv light chain CDR3 |
| 298 | QLQLQESGPGLVKPSETLSLTCTVS | Anti-BCMA CT103A |
| | GGSISSSSYYWGWIRQPPGKGLEWI | scFv heavy chain |
| | GSISYSGSTYYNPSLKSRVTISVDT | variable region |
| | SKNQFSLKLSSVTAADTAVYYCARD | |
| | RGDTILDVWGQGTMVTVSS | |
| 299 | GGSISSSSYY | Anti-BCMA CT103A |
| | | scFv heavy chain |
| | | CDR1 |
| 300 | ISYSGST | Anti-BCMA CT103A |
| | | scFv heavy chain |
| | | CDR2 |
| 301 | ARDRGDTILDV | Anti-BCMA CT103A |
| | | scFv heavy chain |
| | | CDR3 |

In certain embodiments comprising an anti-FAP CAR, the anti-FAP CAR comprises as scFv based on the antibody 4G5 (see WO2021/061708 and WO2021/061778). In some embodiments comprising an anti-FAP CAR comprising an scFv based on the antibody 4G5 further comprises a hinge and transmembrane from CD8, a 4-1 BB co-stimulatory domain, and a CD3ζ signaling domain. Examples of an anti-FAP CARs include CARs disclosed in WO2021/061778.

TABLE 14
Exemplary sequences of anti-FAP 4G5-based CARs

| SEQ ID NO: | Amino Acid Sequence | Description |
|---|---|---|
| 302 | MALPVTALLLPLALLLHAARPGS | Signal peptide |
| 303 | QVQLQQPGAELVKPGASVKLSCKASG | 4G5 scFv VH N- |
| | YTITSYSLHWVKQRPGQGLEWIGEIN | terminal |
| | PANGDHNFSEKFEIKATLTVDSSSNT | |
| | AFMQLSRLTSEDSAVYYCTRLDDSRF | |
| | HWYFDVWGAGTTVTVSSGGGGSGGGG | |
| | SGGGGSQIVLTQSPALMSASPGEKVT | |
| | MTCTASSSVSYMYWYQQKPRSSPKPW | |
| | IFLTSNLASGVPARFSGRGSGTSFSL | |
| | TISSMEAEDAATYYCQQWSGYPPITF | |
| | GSGTKLEIK | |
| 304 | SGTTTPAPRPPTPAPTIASQPLSLRP | CD8α hinge |
| | EACRPAAGGAVHTRGLDFACD | (preceded by |
| | | SG spacer |
| 235 | IYIWAPLAGTCGVLLLSLVITLYC | CD8α |
| | | transmembrane |
| | | domain |
| 237 | KRGRKKLLYIFKQPFMRPVQTTQEED | 4-1BB co- |
| | GCSCRFPEEEEGGCEL | stimulatory |
| | | domain |
| 240 | RVKFSRSADAPAYKQGQNQLYNELNL | Cd3ζ signaling |
| | GRREEYDVLDKRRGRDPEMGGKPRRK | domain |
| | NPQEGLYNELQKDKMAEAYSEIGMKG | |
| | ERRRGKGHDGLYQGLSTATKDTYDAL | |
| | HMQALPPR | |
| 305 | QIVLTQSPALMSASPGEKVTMTCTAS | 4G5 scFv VL N- |
| | SSVSYMYWYQQKPRSSPKPWIFLTSN | terminal |
| | LASGVPARFSGRGSGTSFSLTISSME | |
| | AEDAATYYCQQWSGYPPITFGSGTKL | |
| | EIKGGGGSGGGGSGGGGSQVQLQQPG | |
| | AELVKPGASVKLSCKASGYTITSYSL | |
| | HWVKQRPGQGLEWIGEINPANGDHNF | |
| | SEKFEIKATLTVDSSSNTAFMQLSRL | |
| | TSEDSAVYYCTRLDDSRFHWYFDVWG | |
| | AGTTVTVSS | |

In some embodiments, the mRNA encoding an anti-GPRC5D chimeric antigen receptor (CAR). GPRC5D is a G protein-coupled receptor without known ligands and of unclear function in human tissue. However, this receptor is expressed in myeloma cell lines and in bone marrow plasma cells from patients with multiple myeloma. GPRC5D has been identified as an immunotherapeutic target in multiple myeloma and Hodgkin lymphomas. Examples of anti-GPRC5D CARs include those incorporating a GPRC5D binding moiety such as MCARH109 (Mailankody et al., N Engl J Med. 387(13): 1196-1206 (2022)), BMS-986393, or OriCAR-017 (Rodriguez-Otero et al., Blood Cancer J. 14(1): 24 (2024)). Examples of anti-GPRC5D CARs include those incorporating a GPRC5D binding moiety derived from an antibody specific to GPRC5D, for example, talquetamab (Pillarisetti et al., Blood 135-1232-43 (2020)), or forimtamig. In some embodiments, the extracellular binding domain of the anti-GPRC5D CAR comprises an scFv derived from a 6D9 Mouse antibody with specificity to human GPRC5D (see creative-biolabs.com/car-t/anti-gprc5d-6d9-h-41 bb-cd3-car-pcdcar1-26380.htm). In some embodiments, the extracellular binding domain of the GPRC5D CAR comprises an scFv of anti-GPRC5D antibody linked to 4-1 BB or CD28 costimulatory domain and CD3ζ signaling domain as described in Mailankody et al., N Engl J Med. 387(13): 1196-1206 (2022); creative-biolabs.com/car-t/anti-gprc5d-6d9-h-41bb-cd3-car-pcdcar1-26380.htm; and Rodriguez-Otero et al., Blood Cancer J. 14(1): 24 (2024). The entire contents of each of foregoing references in this paragraph are incorporated by reference for all that they teach about the design, structure, and activity of anti-GPRC5D CARs and anti-GPRC5D antibodies that can provide an antigen binding domain for a CAR or immune cell engager, and each example constitutes a means for binding GPRC5D. In any of the aforementioned tLNP embodiments, certain embodiments include tLNPs encapsulating a GPRC5D CAR payload encoded by RNA and having a T cell targeting moiety, such as an anti-CD8 antibody.

In some embodiments, the mRNA encoding an anti-FCRL5 chimeric antigen receptor (CAR). FCRL5 (Fc receptor-like 5), also known as FCRH5, BXMAS1, CD307, CD307E, and IRTA2, is a protein marker expressed on the surface of plasma cells in patients with multiple myeloma. Furthermore, contact with FCRL5 stimulates B-cell proliferation; thus, FCRL5 has been identified as an immunotherapeutic target for this disease. Examples of anti-FCRL5 CARS include those incorporating an FCRL5 binding moiety, such as those described in WO2016090337, WO2017096120, WO2022263855, and WO2024047558. In some embodiments, the extracellular binding domain of the anti-FCRL5 CAR comprises an scFv with specificity to FCRL5, such as ET200-31, ET200-39, ET200-69, ET200-104, ET200-105, ET200-109, or ET200-117. In some embodiments, the extracellular binding domain of the anti-FCRL5 CAR comprises an scFv derived from a mouse antibody with specificity to human FCRL5. Such antibodies include 7D11, F25, F56, and F119, as described in Polson et al., Int. Immunol., 18(9): 1363-1373 (2006); Franco et al., J. Immunol. 190(11): 5739-5746 (2013); Ise et al., Clin. Cancer Res. 11(1): 87-96 (2005); and Ise et al., Clin. Chem. Lab. Med. 44(5): 594-602 (2006), all of which are incorporated by reference herein. In some embodiments, the extracellular binding domain of the anti-FCRL5 CAR comprises a binding moiety derived from the antigen binding domain of an anti-FCRL5 antibody or nanobody, including cevostamab, 2A10H7, 307307, 2A10D6, 13G9, 10A8, 509f6, EPR27365-87, EPR26948-19, or EPR26948-67, or as disclosed in WO2016090337, WO2017096120, WO2022263855, or WO2024047558. In some embodiments, the extracellular binding domain of the anti-FCRL5 CAR comprises a binding moiety derived from an antibody-drug conjugate targeting FCRL5, such as those described in Elkins et al., Mol. Cancer Ther. 11(10): 2222-2232 (2012). In some embodiments, the extracellular binding domain of the anti-FCRL5 CAR is linked to a costimulatory domain, such as a 4-11BB or CD28 costimulatory domain, and a signaling domain, such as a CD3ζ signaling domain. The entire contents of each of the foregoing references in this paragraph are incorporated by reference for all that they teach about the design, structure, properties, and activity of anti-FCRL5 CARs and anti-FCRL5 antibodies that can provide an antigen binding domain for a CAR or immune cell engager. Each example constitutes a means for binding FCRL5. In any of the aforementioned tLNP embodiments, certain embodiments include tLNPs encapsulating a FCRL5 CAR payload encoded by RNA and having a T cell targeting moiety, such as an anti-CD8 antibody.

Each of the CARs with specificity for a particular antigen described herein constitute means for antigen recognition with respect to that antigen and collectively all of the CARs described herein constitute means for antigen recognition. The function can be alternatively stated as antigen recognition by an immune cell or antigen recognition by a T cell and the like.

In certain embodiments, on ORF can encode a gene-editing nuclease such as one encoding an RNA-guided nuclease, a gene or base editing protein, a prime editing protein, a Gene Writer protein (e.g., a modified or modularized non-long terminal repeat (LTR) retrotransposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-MLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Genome-, gene-, and base-editing technology are reviewed in Anzalone et al., Nature Biotechnology 38:824-844, 2020, Sakuma, Gene and Genome Editing 3-4:100017, 2022, and Zhou et al., MedComm 3(3):e155, 2022, each of which is incorporated by reference for all that they teach about the components and uses of this technology to the extent that it does not conflict with the present disclosure.

In certain embodiments of any of the above aspects, the poly(A) sequence can have at least about 80 adenosine residues to about 130 or more adenosine residues. In some embodiments the poly(A) sequence has about 80 adenosine residues. In certain embodiments, the poly(A) sequence has about 90 adenosine residues. In certain embodiments, the poly(A) sequence has about 100 adenosine residues. In certain embodiments, the poly(A) sequence has about 110 adenosine residues. In certain embodiments, the poly(A) sequence has about 130 adenosine residues.

(II)(d)(3) CAR Constructs Used in the mRNA Screening

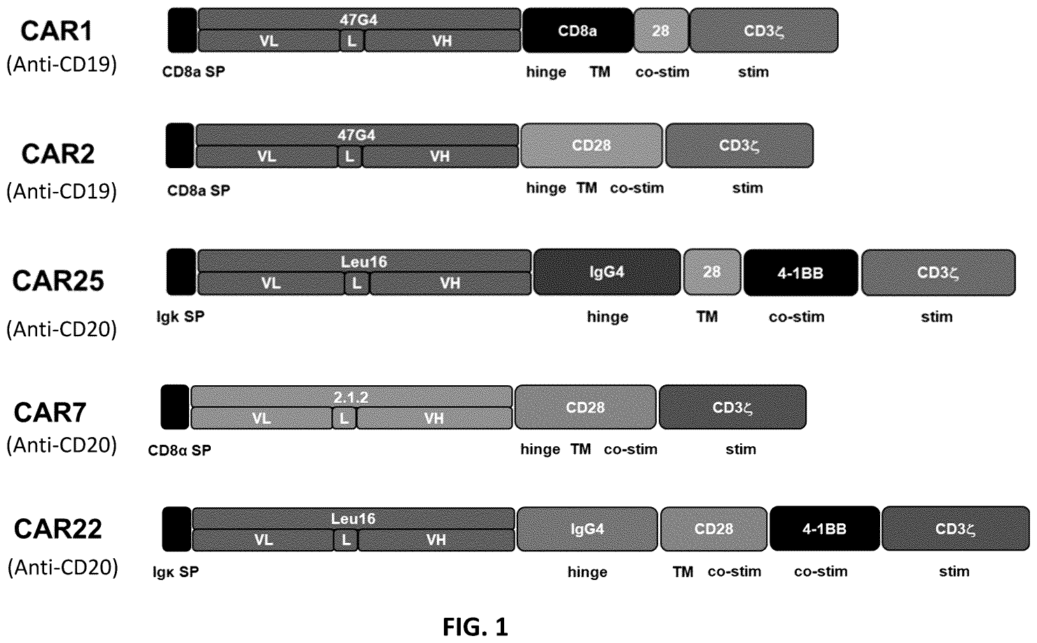

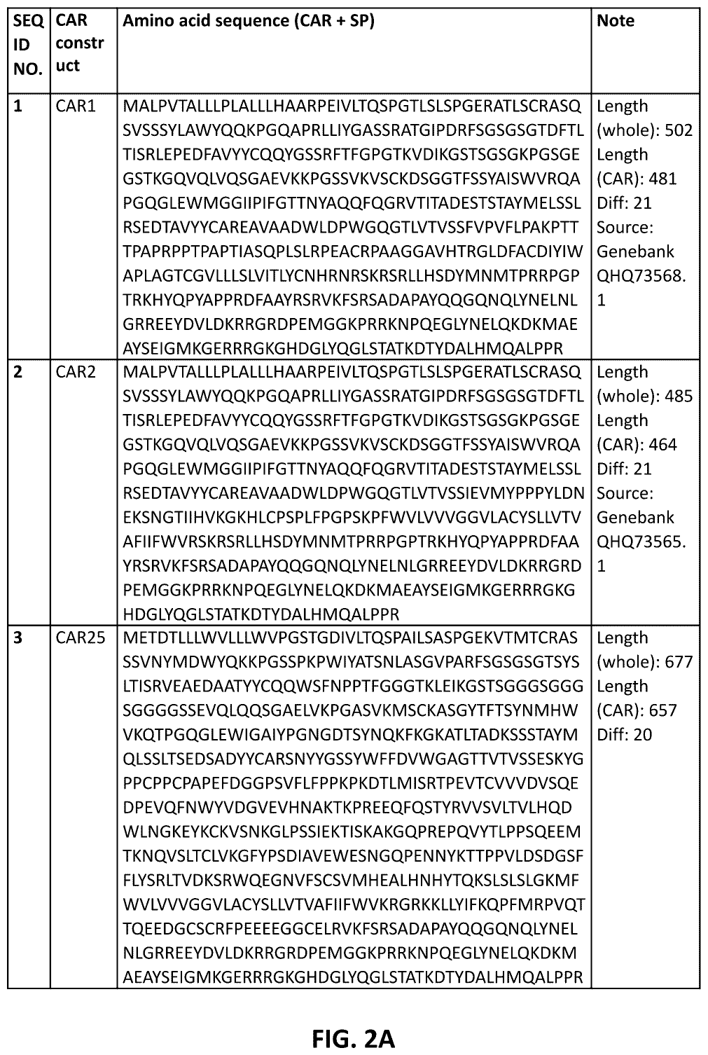

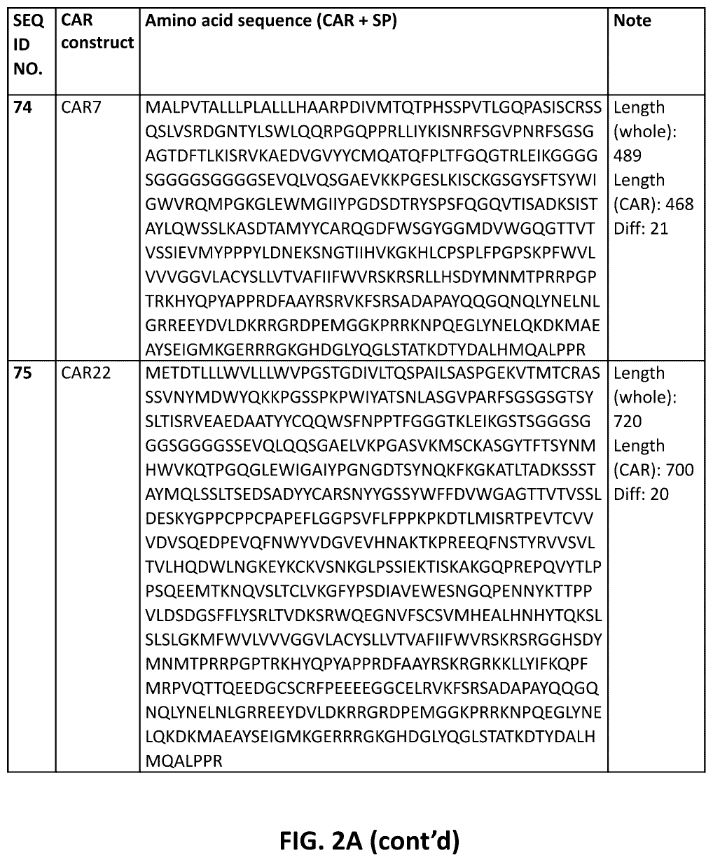

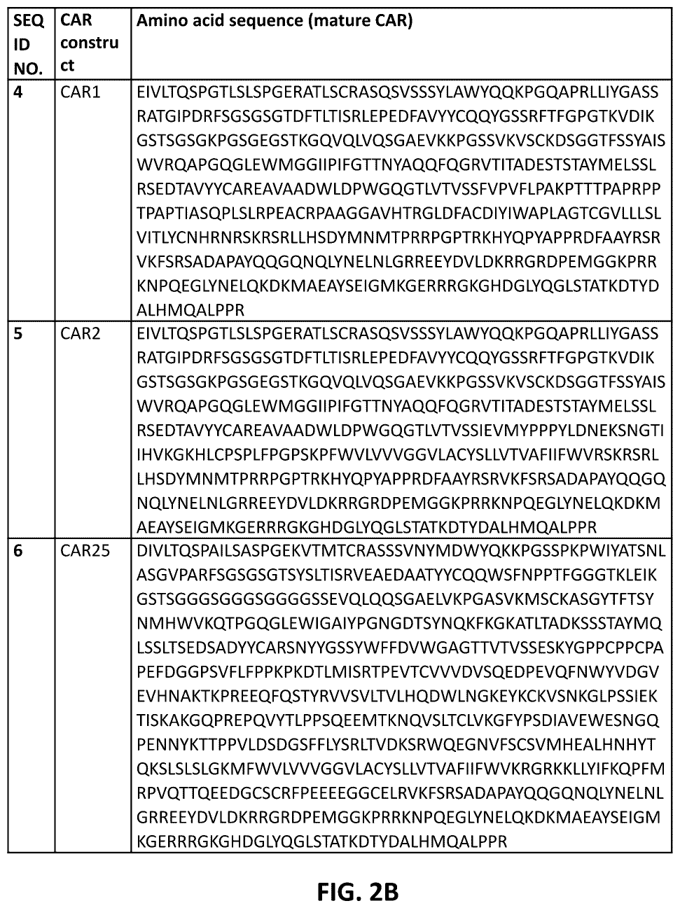

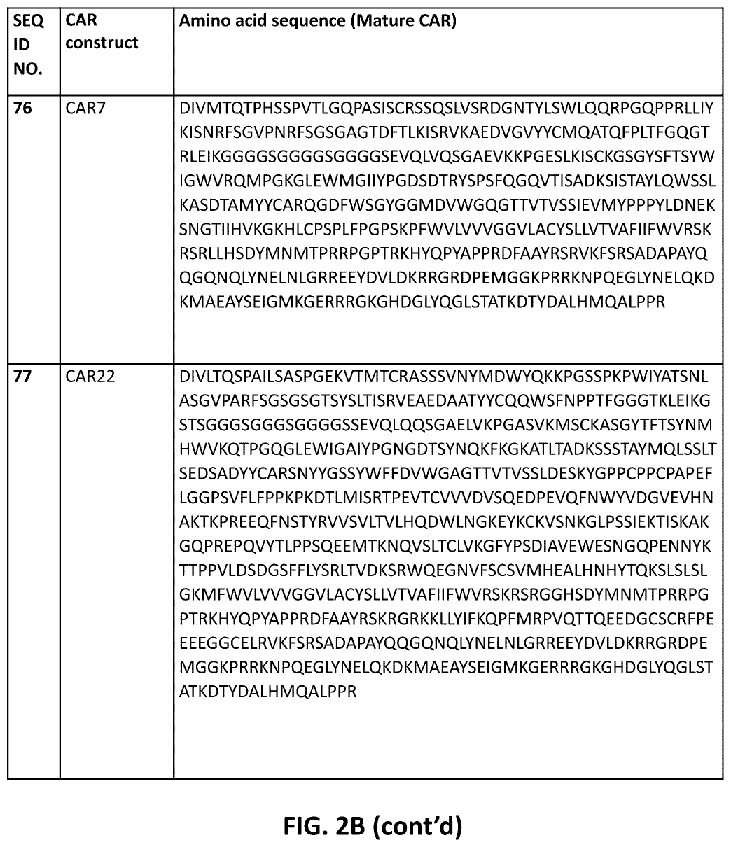

In certain embodiments, two CAR configurations are used for anti-CD19 CAR: CAR1 and CAR2. CAR1 mRNAs encode an amino acid sequence (SEQ ID: 1, FIG. 2A) comprising the following domains in N- to C-terminal order: CD8a signal peptide (SP), anti-CD19 scFv derived from mAb 47G4 (light chain variable domain, VL; linker, L; heavy chain variable domain, VH; 47G4 is disclosed in US2010/0104509), CD8a hinge, CD8a transmembrane domain (TM), CD28 costimulatory domain (co-stim), and CD3ζ signaling domain (stim) (FIG. 1). The CAR1 amino acid sequence is disclosed in U.S. Pat. No. 10,287,350 (WO2015/187528) as SEQ ID NO: 1, from which the CAR1 amino acid sequence and its synthesis are incorporated herein by reference. The amino acid sequence of the mature CAR1 protein (i.e., without a signal peptide) is provided as SEQ ID NO: 4 (FIG. 2B). The incorporation of the CD8α hinge and transmembrane domains in CAR1 helps reduce cytokine release syndrome (cytokine storm) in comparison to similar anti-CD19 CAR molecules that instead incorporate a CD28 hinge and transmembrane domain when CAR-T cells are generated ex vivo and adoptively transferred.

In certain embodiments, the CAR2 mRNAs that are used encode an amino acid sequence (SEQ ID: 2, FIG. 2A) comprising the following domains in N- to C-terminal order: CD8a signal peptide (SP), anti-CD19 scFv derived from mAb 47G4 (light chain variable domain, VL; linker, L; heavy chain variable domain, VH), CD28 hinge, CD28 transmembrane (TM), CD28 co-stimulatory domain (co-stim), and CD3ζ signaling domain (stim) as shown in FIG. 1. The amino acid sequence of the mature CAR2 protein (i.e., without a signal peptide) is provided as SEQ ID NO: 5 (FIG. 2B). Combining the 47G4 scFv with the CD28 hinge and transmembrane domains provides CAR2 an advantage for transient in vivo transfection as opposed to the traditional CAR-T cell comprising an integrated DNA sequence encoding CAR. CAR2 is expressed at a higher level than CAR1 from mRNAs using the same UTRs and codon optimization method and the T cells expressing CAR2 eliminate more CD19⁺ cells.

In certain embodiments, two CAR configurations are used for anti-CD20 CAR: CAR25, CAR7, and CAR22. The CAR25 mRNA encodes an amino acid sequence (SEQ ID: 4, FIG. 2A) comprising the following domains in N- to C-terminal order: Ig-kappa signal peptide (IgK SP), anti-CD20 scFv derived from the Leu16 mAb (light chain variable domain, VL; linker, L; heavy chain variable domain, VH), IgG4 hinge, CD28 transmembrane domain (TM), 4-1 BB co-stimulatory domain (co-stim), and CD3 ζ signaling domain (stim) as shown in FIG. 1. The amino acid sequence of the mature CAR25 protein (i.e., without a signal peptide) is provided as SEQ ID NO: 6 (FIG. 2B).

In certain embodiments, CAR7 mRNA encodes an amino acid sequence (SEQ ID: 74, FIG. 2A) comprising the following domains in N- to C-terminal order: CD8α signal peptide (SP), anti-CD20 scFv derived from 2.1.2 hybridoma (light chain variable domain, VL; and heavy chain variable domain, VH) and (G₄S)₃ linker (GGGGSGGGGSGGGGS—SEQ ID NO: 252), CD28 hinge, transmembrane domain (TM), and co-stimulatory domain (co-stim), and CD3ζ signaling domain (stim) as shown in FIG. 1. The amino acid sequence of the mature CAR7 protein (i.e., without a signal peptide) is provided as SEQ ID NO: 76 (FIG. 2B).

In certain embodiments, CAR22 mRNA encodes an amino acid sequence (SEQ ID: 75, FIG. 2A) comprising the following domains in N- to C-terminal order: mouse Ig-kappa signal peptide (IgK SP), anti-CD20 scFv derived from the Leu16 mAb (light chain variable domain; heavy chain variable domain, VH) and (G₃S)₂-G₄S linker (GSTSGGGSGGGSGGGGSS—SEQ ID NO: 229), IgG4 hinge, CD28 transmembrane domain (TM) and co-stimulatory domain (co-stim), 4-1BB co-stimulatory domain (co-stim), and CD3ζ signaling domain (stim) as shown in FIG. 1. The amino acid sequence of the mature CAR22 protein (i.e., without a signal peptide) is provided as SEQ ID NO: 77 (FIG. 2B).

In certain embodiments, the ORFs encoding CAR1, CAR2, CAR7, CAR22, and CAR25 are codon optimized according to the strategies (OPT1-OPT8) described in section (II)(d)(1) above. Note that the SEQ ID NOs below list the sequences without the stop codons. In certain embodiments, the ORF is:
CAR1-OPT2 (SEQ ID NO: 90), CAR1-OPT3 (SEQ ID NO: 91), CAR1-OPT6 (SEQ ID NO: 92), CAR1-OPT4 (SEQ ID NO: 93), CAR1-OPT7 (SEQ ID NO: 94), CAR1-OPT1 (SEQ ID NO: 95), CAR1-OPT5 (SEQ ID NO: 96), or a sequence having at least 95, 96, 97, 98, or 99% sequence identity thereto;
CAR2-OPT2 (SEQ ID NO: 97), CAR2-OPT3 (SEQ ID NO: 98), CAR2-OPT6 version 1 (SEQ ID NO: 99), CAR2-OPT6 version 2 (SEQ ID NO: 100), CAR2-OPT4 (SEQ ID NO: 101), CAR2-OPT7 (SEQ ID NO: 102), CAR2-OPT1 (SEQ ID NO: 103), or CAR2-OPT5 (SEQ ID NO: 104), or a sequence having at least 95, 96, 97, 98, or 99% sequence identity thereto;
CAR25-OPT6 (SEQ ID NO: 105), or CAR25-OPT4 (SEQ ID NO: 106), or a sequence having at least 95, 96, 97, 98, or 99% sequence identity thereto;
CAR7-OPT0 (SEQ ID NO: 78), CAR7-OPT4 (SEQ ID NO: 79), or CAR7-OPT6 (SEQ ID NO: 80), or a sequence having at least 95, 96, 97, 98, or 99% sequence identity thereto; or
CAR22-OPT0 (SEQ ID NO: 81), CAR22-OPT4 (SEQ ID NO: 82), or CAR22-OPT8 (SEQ ID NO: 83), or a sequence having at least 95, 96, 97, 98, or 99% sequence identity thereto.
   (II)(e) Improved mRNA Constructs Containing a 5′ UTR, ORF, and 3′ UTR

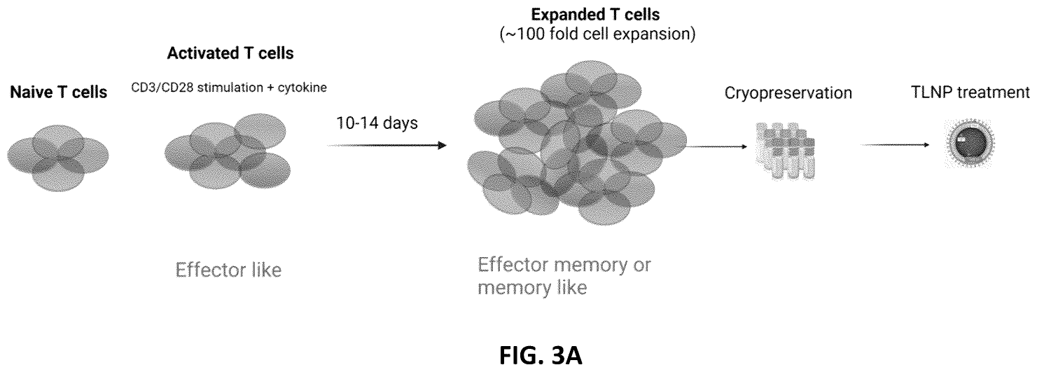

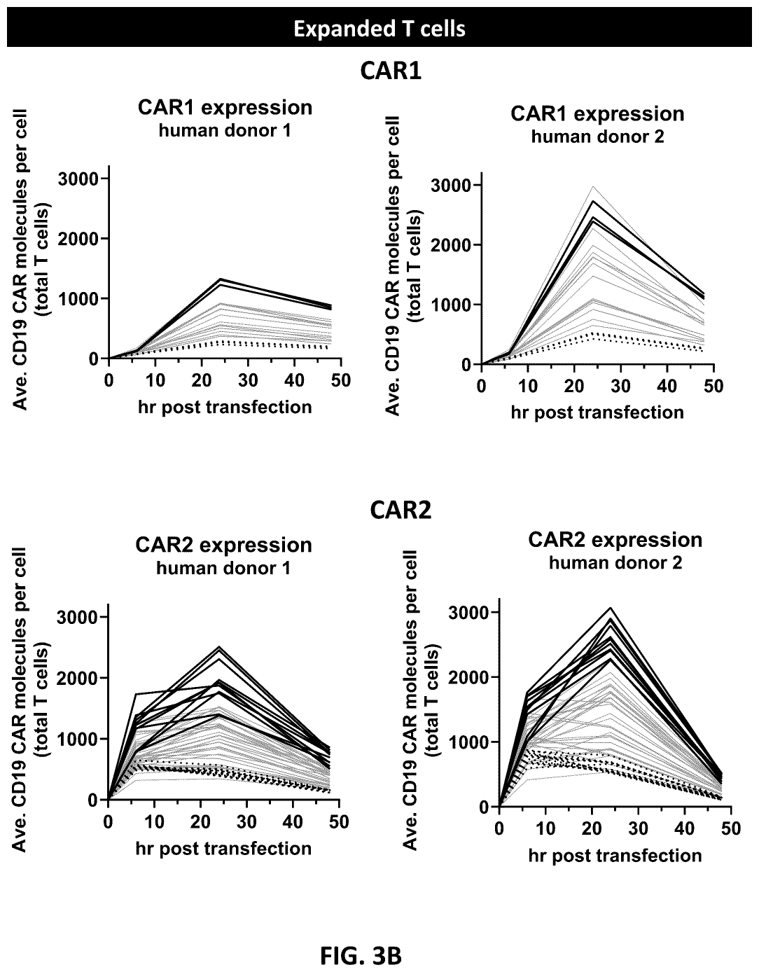

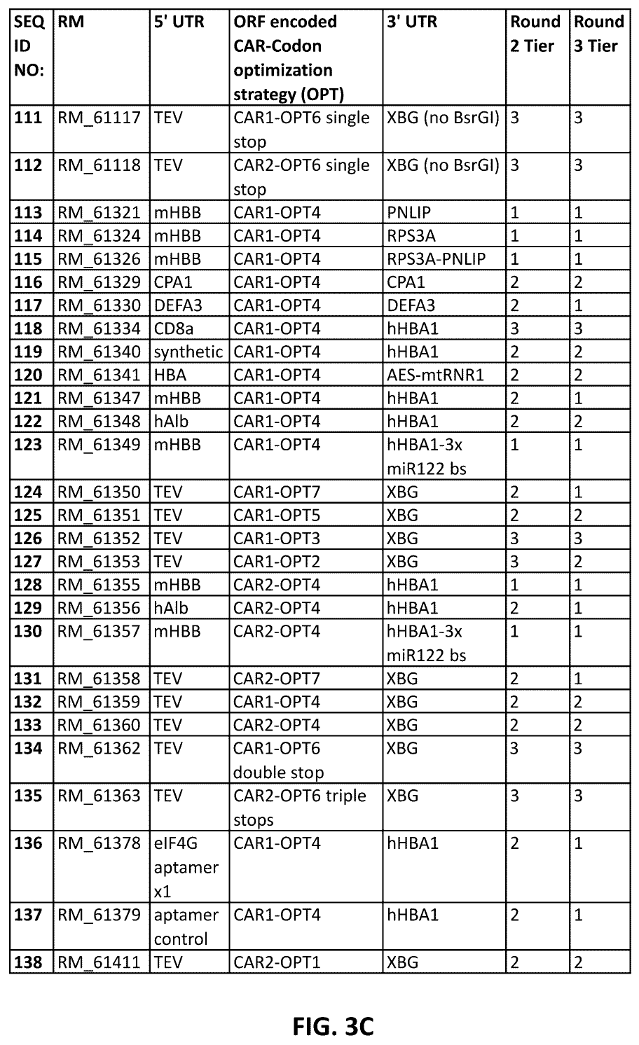

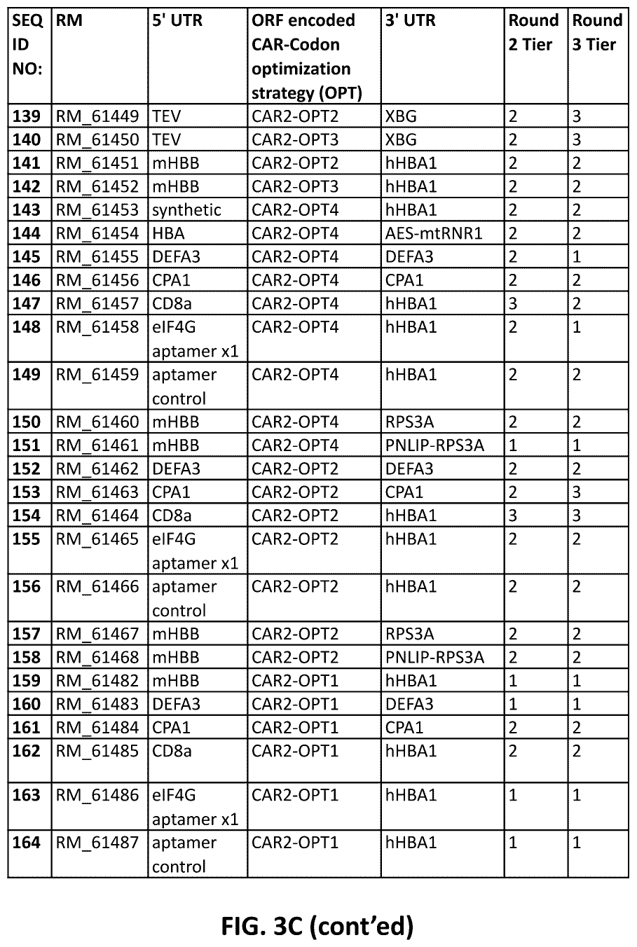

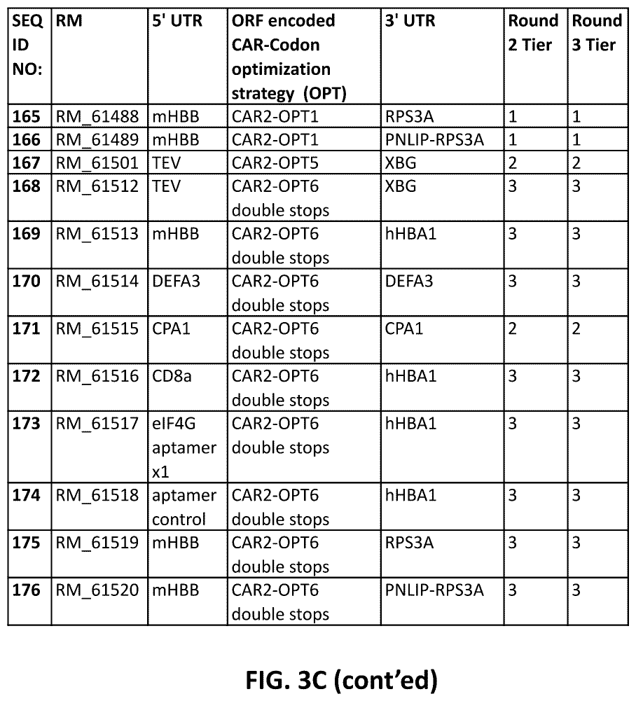

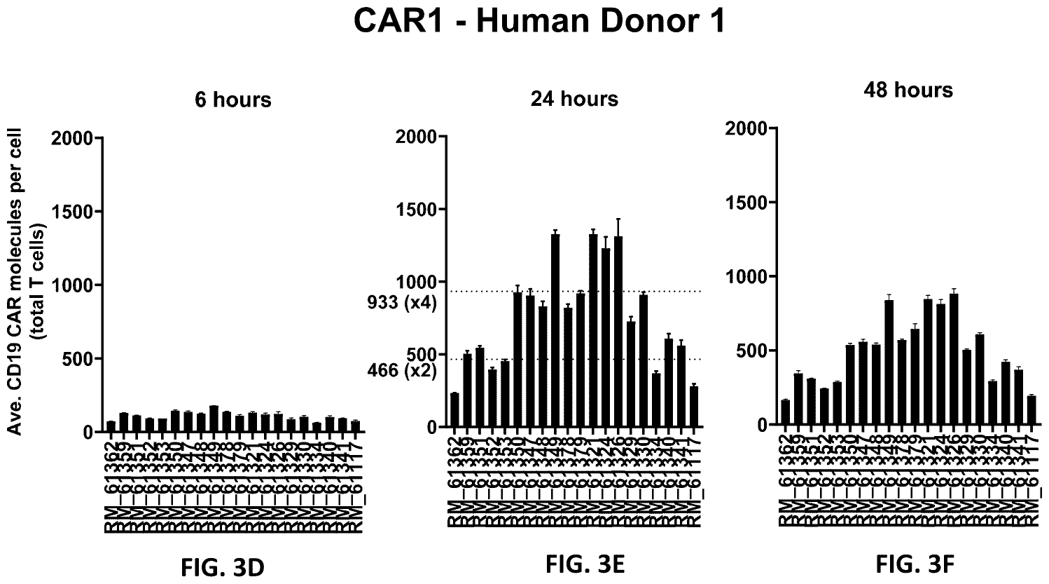

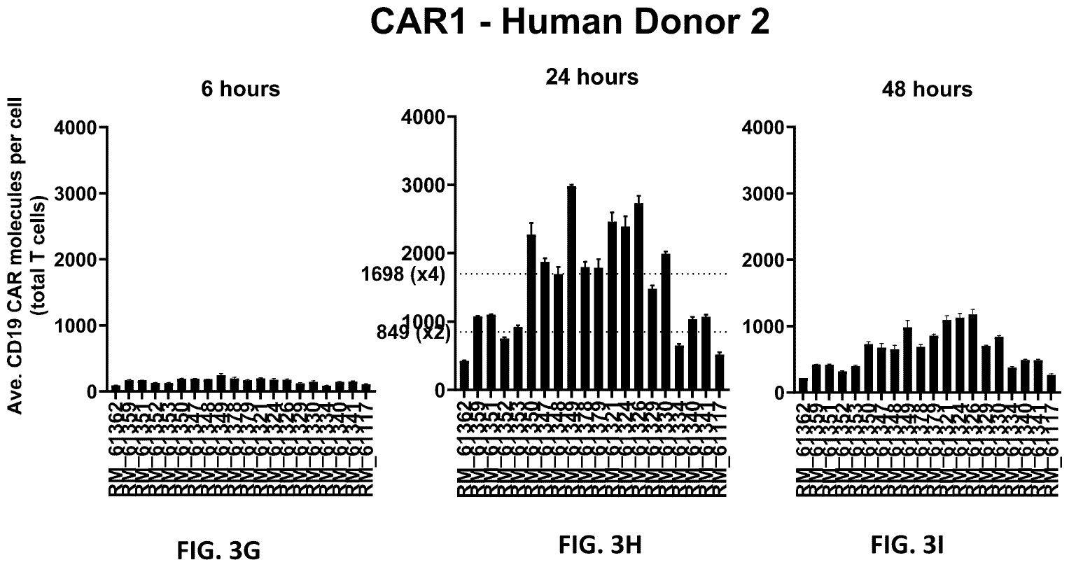

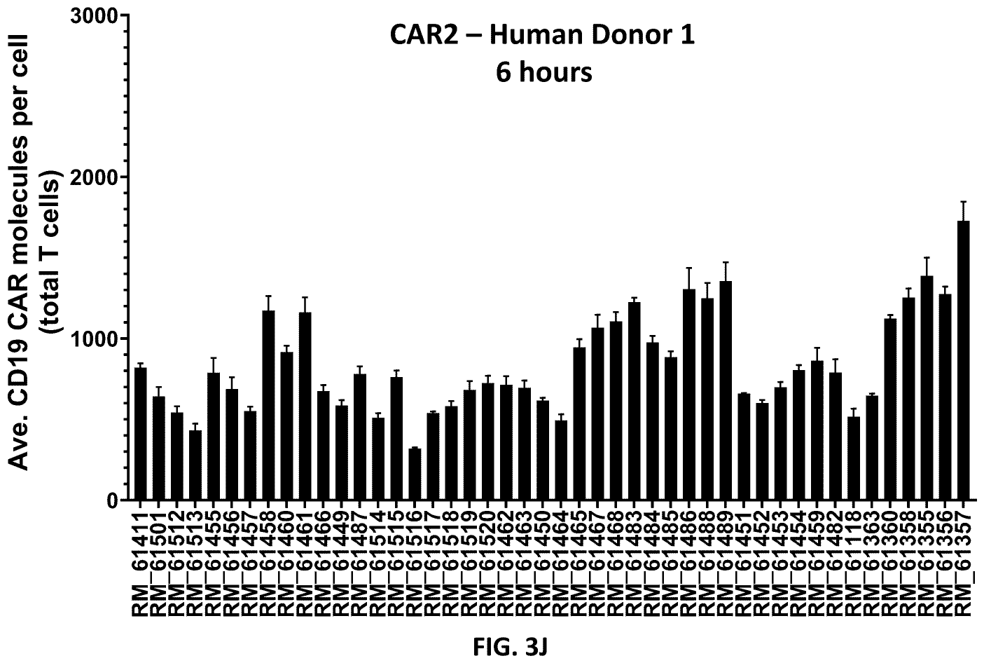

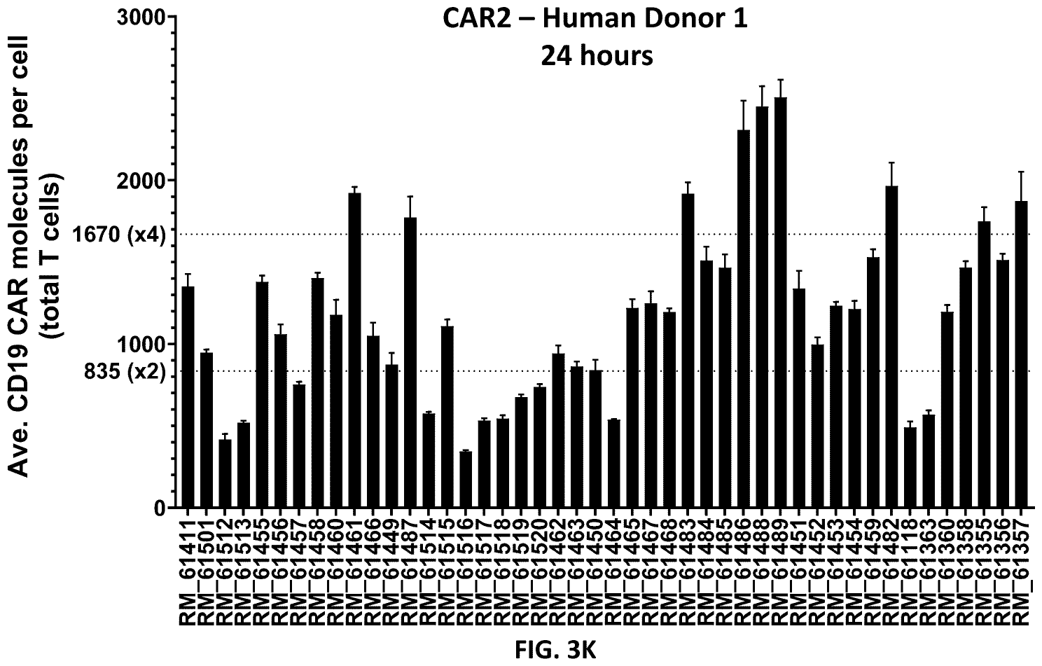

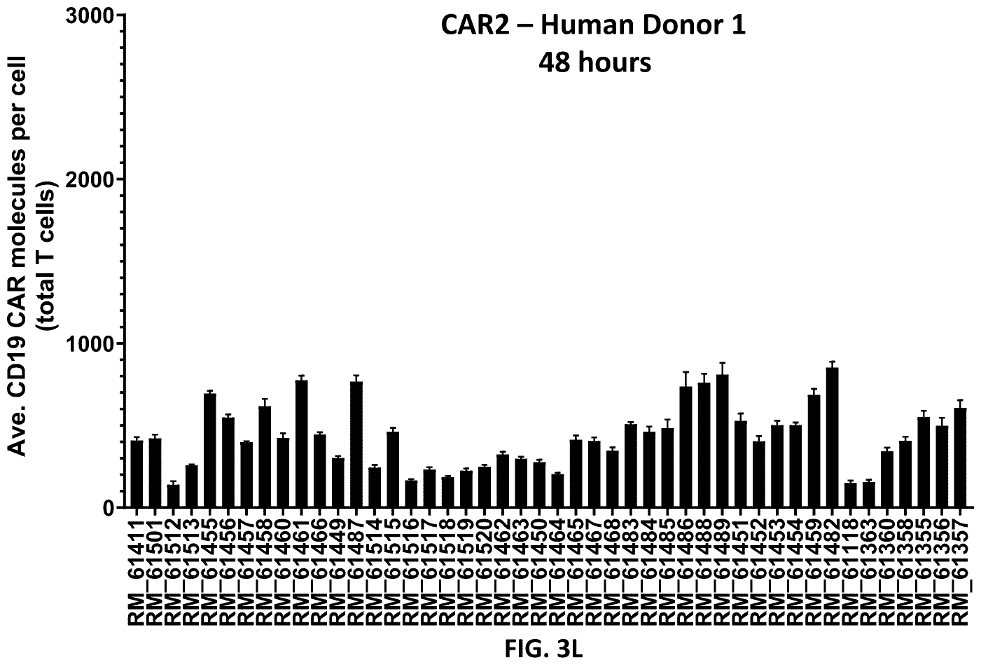

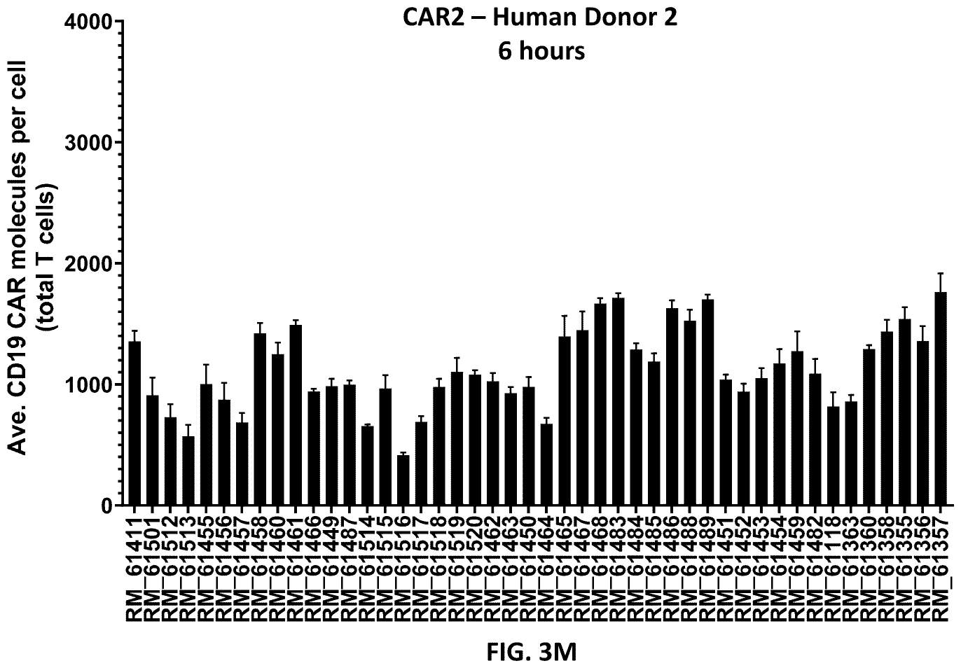

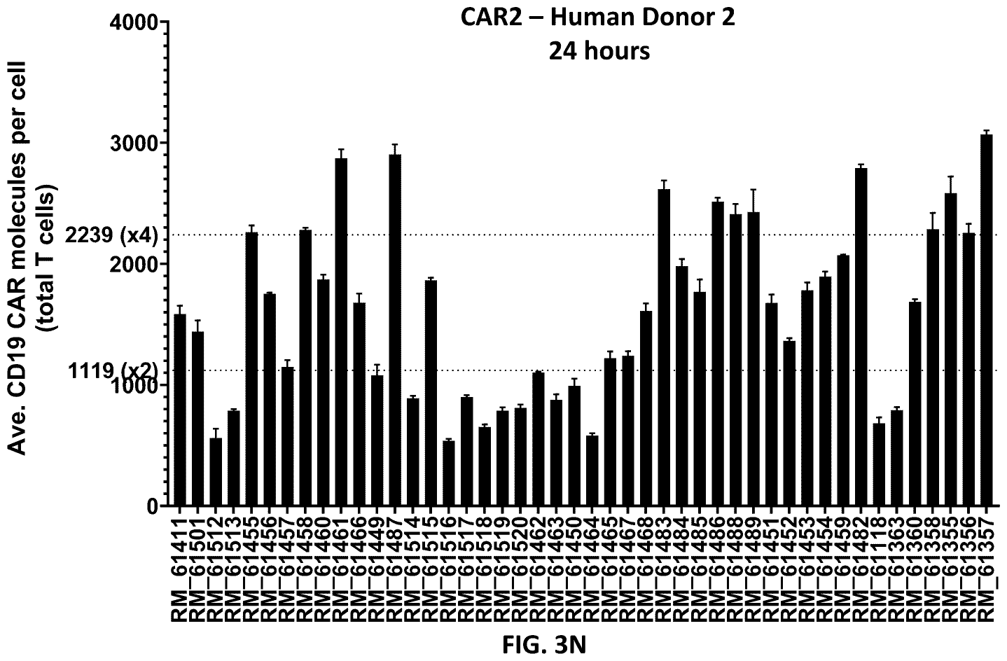

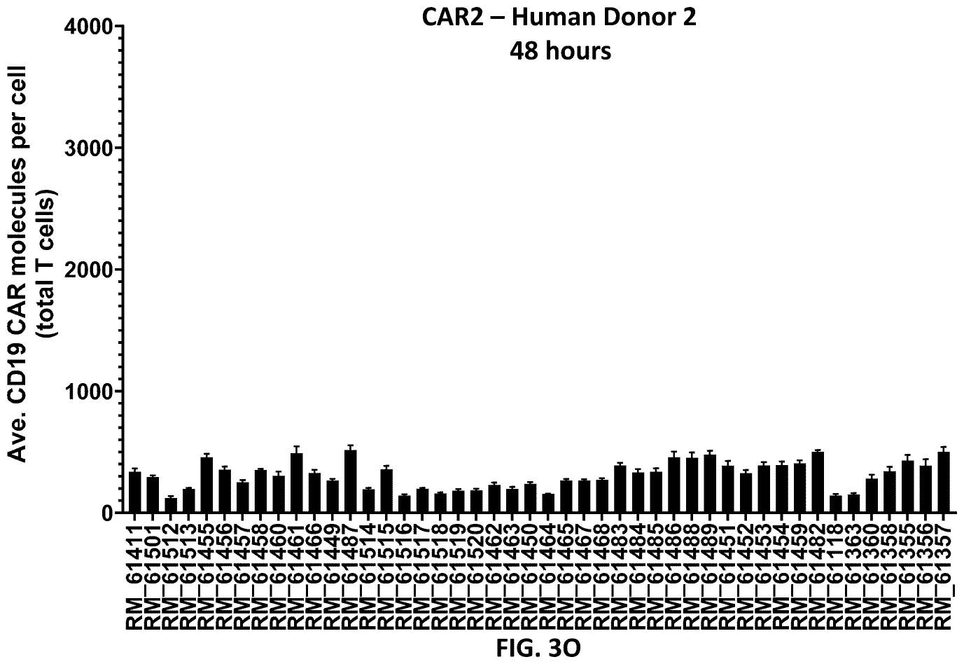

Provided herein are mRNAs comprising optimized 5′ UTR and 3′ UTR pairs, and/or ORF encoding CAR, which are effectively transfected into cells both in vitro and in vivo. FIG. 3C contains brief description of all mRNAs (labelled by RM_xxxxx) described herein. In certain embodiments, the mRNA is RM_61321, RM_61324, RM_61326, RM_61329, RM_61330, RM_61334, RM_61340, RM_61341, RM_61347, RM_61348, RM_61349, RM_61350, RM_61351, RM_61352, RM_61353, RM_61355, RM_61356, RM_61357, RM_61358, RM_61359, RM_61360, RM_61378, RM_61379, RM_61411, RM_61449, RM_61450, RM_61451, RM_61452, RM_61453, RM_61454, RM_61455, RM_61456, RM_61457, RM_61458, RM_61459, RM_61460, RM_61461, RM_61462, RM_61463, RM_61465, RM_61466, RM_61467, RM_61468, RM_61482, RM_61483, RM_61484, RM_61485, RM_61486, RM_61487, RM_61488, RM_61489, RM_61501, RM_61514, RM_61515, RM_61519, RM_61520, RM_61639, RM_61653, RM_61654, RM_61655, RM_61656, RM_61657, RM_61658, RM_61659, or RM_61660 (SEQ ID NO: 113-133, 136-153, 155-167, 170, 171, 175, 176, 178, 316-319, and 321-324 respectively).

In certain embodiments, the mRNA is RM_61321, RM_61324, RM_61326, RM_61329, RM_61330, RM_61340, RM_61341, RM_61347, RM_61348, RM_61349, RM_61355, RM_61356, RM_61357, RM_61378, RM_61379, RM_61451, RM_61452, RM_61453, RM_61454, RM_61455, RM_61456, RM_61458, RM_61459, RM_61460, RM_61461, RM_61462, RM_61463, RM_61465, RM_61466, RM_61467, RM_61468, RM_61482, RM_61483, RM_61484, RM_61486, RM_61487, RM_61488, RM_61489, RM_61514, RM_61515, RM_61519, RM_61520, RM_61639, RM_61653, RM_61654, RM_61655, RM_61656, RM_61657, RM_61658, RM_61659, or RM_61660 (SEQ ID NO: 113-117, 119-123, 128-130, 136, 137, 141-146, 148-153, 155-161, 163-166, 170, 171, 175, 176, 178, 316-319, and 321-324 respectively).

In certain embodiments, the mRNAs have an elevated level of expression of the CAR upon tLNP-mediated transfection into mammalian cells compared to a reference mRNA in which the 5′ UTR comprises tobacco etch virus (TEV) 5′ UTR, the ORF comprises the OPT6 sequence (CAR sequence optimized by IDT codon optimization tool), and the 3′ UTR comprises Xenopus β-globin (XBG) 3′ UTR.

In certain embodiments, expression of the mRNAs is at least 2-fold greater than the reference mRNA. Examples of the mRNA include, without limitation, RM_61321, RM_61324, RM_61326, RM_61349, RM_61350, RM_61330, RM_61379, RM_61347, RM_61378, RM_61348, RM_61329, RM_61340, RM_61341, RM_61351, and RM_61359 for CAR1 and RM_61353, RM_61355, RM_61357, RM_61461, RM_61482, RM_61483, RM_61486, RM_61487, RM_61488, RM_61489, RM_61458, RM_61455, RM_61356, RM_61358, RM_61459, RM_61484, RM_61485, RM_61454, RM_61451, RM_61453, RM_61515, RM_61460, RM_61456, RM_61411, RM_61468, RM_61360, RM_61466, RM_61467, RM_61465, RM_61452, RM_61501, RM_61457, RM_61449, RM_61462, RM_61463, and RM_61450 for CAR2.

In certain embodiments, expression of the mRNAs is at least 2-fold greater than the reference mRNA in human expanded T cells. Examples of the mRNA include, without limitation, RM_61321, RM_61324, RM_61326, RM_61349, RM_61350, RM_61330, RM_61379, RM_61347, RM_61378, RM_61348, RM_61329, RM_61340, RM_61341, RM_61351, RM_61353, and RM_61359 for CAR1 and RM_61355, RM_61357, RM_61461, RM_61482, RM_61483, RM_61486, RM_61487, RM_61488, RM_61489, RM_61458, RM_61455, RM_61356, RM_61358, RM_61459, RM_61484, RM_61485, RM_61454, RM_61451, RM_61453, RM_61515, RM_61460, RM_61456, RM_61411, RM_61468, RM_61360, RM_61466, RM_61467, RM_61465, RM_61452, RM_61501, RM_61457, RM_61449, RM_61462, RM_61463, and RM_61450 for CAR2.

In certain embodiments, expression of the mRNAs is at least 2-fold greater than the reference mRNA in human activated T cells. Examples of the mRNA include, without limitation, RM_61321, RM_61324, RM_61326, RM_61349, RM_61350, RM_61330, RM_61379, RM_61347, RM_61378, RM_61348, RM_61329, RM_61359 for CAR1 and RM_61355, RM_61357, RM_61461, RM_61482, RM_61483, RM_61486, RM_61487, RM_61488, RM_61489, RM_61458, RM_61455, RM_61356, RM_61358, RM_61459, RM_61484, RM_61485, RM_61451, RM_61515, RM_61460, RM_61456, RM_61411, RM_61360, and for CAR2.

In certain embodiments, expression of the mRNAs is at least 3-fold greater than the reference mRNA in human activated T cells. Examples of the mRNA include, without limitation, RM_61321, RM_61324, RM_61326, RM_61349, RM_61378, and RM_61379 for CAR1.

In certain embodiments, expression of the mRNAs is at least 2-fold greater than the reference mRNA in human expanded T cells. Examples of the mRNA include, without limitation, RM_61639 for CAR 25, RM_61655 and RM_61656 for CAR22, and RM_61657, RM_61658, RM_61659, and RM_61660 for CAR7.

In certain embodiments, expression of the mRNAs is at least 4-fold greater than the reference mRNA. Examples of the mRNA include, without limitation, RM_61321, RM_61324, RM_61326, RM_61330, RM_61347, RM_61349, RM_61350, RM_61378, and RM_61379 for CAR1 and RM_61355, RM_61357, RM_61461, RM_61482, RM_61483, RM_61486, RM_61487, RM_61488, RM_61489, RM_61458, RM_61455, RM_61356, and RM_61358 for CAR2.

In certain embodiments, expression of the mRNAs is at least 4-fold greater than the reference mRNA in human expanded T cells. Examples of the mRNA include, without limitation, RM_61321, RM_61324, RM_61326, RM_61330, RM_61347, RM_61349, RM_61350, RM_61378, and RM_61379 for CAR1 and RM_61355, RM_61357, RM_61461, RM_61482, RM_61483, RM_61486, RM_61487, RM_61488, RM_61489, RM_61458, RM_61455, RM_61356, and RM_61358 for CAR2.

In certain embodiments, expression of the mRNAs is at least 4-fold greater than the reference mRNA in human activated T cells. Examples of the mRNA include, without limitation, RM_61455, RM_61458, RM_61461, RM_61486, RM_61488, RM_61489, RM_61355, and RM_61357 for CAR2.

In certain embodiments, expression of the mRNAs is at least 5-fold greater than the reference mRNA. Examples of the mRNA include, without limitation, RM_61321, RM_61324, RM_61326, RM_61349, and RM_61350 for CAR1 and RM_61357, RM_61461, RM_61486, RM_61487, RM_61488, and RM_61489 for CAR2.

In certain embodiments, expression of the mRNAs is at least 5-fold greater than the reference mRNA in human expanded T cells. Examples of the mRNA include, without limitation, RM_61321, RM_61324, RM_61326, RM_61349, and RM_61350 for CAR1 and RM_61357, RM_61461, RM_61486, RM_61487, RM_61488, and RM_61489 for CAR2.

In certain embodiments, expression of the mRNAs is at least 5-fold greater than the reference mRNA in human activated T cells. Examples of the mRNA include, without limitation, RM_61357 and RM_61461 for CAR2.

In certain embodiments, the improved mRNA comprises the same UTRs used in the base construct and improved expression is attributable to an improved ORF. Examples of such mRNAs include, without limitation, RM_61358, RM_61359, RM_61360, RM_61501, RM_61411, RM_61449, RM_61450, RM_61350, RM_61351, RM_61352, and RM_61353.

Certain embodiments comprise individual mRNA molecules or groupings thereof wherein one or more mRNA molecules has at least 95, 96, 97, 98, 99, or 100% identity to any of the foregoing mRNA molecules.

In certain embodiments, the sequences of the above mRNAs are comprised in other nucleic acid molecules such as circular RNA molecules, saRNA molecules, or DNA molecules encoding any of such (m)RNA molecules. The encoding DNA molecules can comprise additional sequences to facilitate in vitro transcription, circularization, or self-amplification as disclosed herein or known in the art.

In certain instances, the nucleic acid disclosed herein is a “nucleoside-modified nucleic acid,” which refers to a nucleic acid comprising at least one modified nucleoside. A “modified nucleoside” refers to a nucleoside with a chemical modification relative to the standard nucleosides such as uridine, thymidine, ribonucleosides, and 2′-deoxyribonucleosides of adenine, guanine, and cytosine. For example, over one hundred different nucleoside modifications have been identified in RNA (Rozenski et al., 1999, The RNA Modification Database: 1999 update. Nucl Acids Res 27: 196-197).

In some embodiments, some or all of the uridines of the mRNA have been replaced with one or more types of a pseudouridine, or other modified nucleoside(s). In certain embodiments, “a pseudouridine” refers to N1-methyl pseudouridine (N1Mψ or m¹ψ). In certain embodiments, “a pseudouridine” refers to 5-methoxyuridine (5moU), In another embodiment, “a pseudouridine” refers to m¹acp³Y (1-methyl-3-(3-amino-3-carboxypropyl) pseudouridine. In another embodiment, the term refers to m¹Y (1-methylpseudouridine). In another embodiment, the term refers to Ym (2′-O-methylpseudouridine. In another embodiment, the term refers to m⁵D (5-methyldihydrouridine). In another embodiment, the term refers to m³Y (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. In some embodiments, cytidines are substituted with a modified nucleoside, such as 5-methyl cytidine, instead of or in addition to uridines. Each possibility represents a separate embodiment of the herein disclosed inventions. Further modified nucleosides are disclosed in WO2007024708 which is incorporated by reference in its entirety for all that it teaches about modified nucleosides and their use in RNA.

(III) mRNA Synthesis

In certain embodiments, the mRNA sequences can be synthesized by in vitro transcription (IVT). Such in vitro transcription can be accomplished using a phage RNA polymerase such as T7 RNA polymerase and beneficially provides substitution of uridine with a pseudouridine (e.g., without limitation, N1-methylpseudouridine (N1Mψ) or 5-methoxyuridine (5moU)) (e.g., as shown in Example 1). In other embodiments, cytidine is substituted with 5-methylcytidine instead of or in addition to U substitution. Templates used in such IVT method comprise linear DNA molecules comprising the 5′ UTR, ORF and 3′ UTR sequences inserted between a T7 RNA polymerase promoter and a poly(A) tail (e.g., about 90 to about 110 adenine residues) in which the poly(A) tail is at the 3′ end of the linear DNA. For each genus and species of RNA molecule(s) disclosed herein there is a genus or species of template DNA molecule(s) encoding the RNA molecule(s). Various restriction sites can be used for the insertion. Examples of restriction sites include, without limitation, EcoR1, BamH1, and BsrG1. Typically, template DNA is produced as a bacterial plasmid carrying a selectable marker such as kanamycin resistance. Circular plasmids are linearized at a unique type II restriction enzyme site, located downstream of the poly(A) tail. The linearized plasmid serves as template for IVT. The IVT process can utilize T7 RNA polymerase in reaction conditions known in the art and partial or complete substitution of uridine with a pseudouridine (e.g., without limitation, N1Mψ or 5moU). A Cap1 structure can be added co-transcriptionally using, for example, a cap-AG trinucleotide reagent (for example, m7G(5′)ppp(5′)(2′OMeA)pG). In alternative embodiments, template DNA comprises a T3 or SP6 RNA polymerase promoter instead of the T7 RNA polymerase promoter and the corresponding polymerase is used to synthesize the mRNA.

(IV) Cells Comprising the mRNAs and/or Polypeptide Encoded by the mRNA.

In certain other aspects, this disclosure provides a cell comprising any of the mRNAs disclosed herein, wherein the cell does not comprise DNA encoding the mRNA. Similarly, in further embodiments, this disclosure provides a cell comprising an mRNA encoding a polypeptide disclosed herein, wherein the cell does not comprise DNA encoding the mRNA. In various embodiments, the polypeptide is a CAR disclosed herein.

In certain other aspects, this disclosure provides a cell comprising a polypeptide encoded by any of the mRNAs disclosed herein, wherein the cell does not comprise DNA encoding the mRNA. In certain embodiments, the polypeptide is a CAR. In certain embodiments, the CAR is an anti-CD19 CAR, an anti-CD20 CAR, an anti-BCMA CAR, or an anti-FAP CAR. In certain embodiments, the polypeptide comprises an RNA-guided nuclease or derivative thereof. In certain instances, the RNA-guided nuclease is Cas9, Cas12, and analogous species thereof, see, e.g., Makarova and Koonin, 2015, Methods Molec. Biol. 1311: 45-47; Tang et al., 2019, Database 1-8. Similarly, in further embodiments, this disclosure provides a cell comprising a polypeptide disclosed herein, wherein the cell does not comprise DNA encoding the mRNA encoding the polypeptide. In various embodiments, the polypeptide is a CAR disclosed herein.

In certain embodiments, the cells are mammalian cells. In certain embodiments, the cells are immune cells. In certain embodiments the immune cells are T cells, NK cells, NKT cells, or monocytes. In certain embodiments, the cell is a hematopoietic stem cell. In certain embodiments, the cell is a mesenchymal stem cell. In certain instances, the cells are human cells.

In certain aspects, this disclosure provides a CAR-modified immune cell (CAR-MIC) comprising a CAR-encoding mRNA and/or the encoded CAR polypeptide of any one of the herein disclosed aspects and embodiments, wherein the cell does not comprise DNA encoding the mRNA. In some embodiments, the CAR-MIC is in the body of a mammal. In some instances, the mammal is a human. In some embodiments, the CAR-MIC has been transfected with the mRNA in vivo. In some embodiments, the CAR-MIC is a T cell. In some embodiments, the CAR-MIC is an NKcell. In some embodiments, the CAR-MIC is an NKT cell. In some embodiments, the CAR-MIC is a monocyte. In certain embodiments, the CAR is an anti-CD19 CAR, an anti-CD20 CAR, an anti-BCMA CAR, or an anti-FAP CAR.

In certain embodiments of these disclosed aspects, an anti-CD19 CAR comprises an anti-CD19 binding domain, CD28 hinge, transmembrane, and co-stimulatory domains, and a CD3ζ signaling domain. In certain embodiments of these aspects, an anti-CD19 CAR comprises an anti-CD19 binding domain, hinge and transmembrane domain from CD8, a CD28 costimulatory domain, and a CD3ζ-chain signaling domain. In certain instances, an anti-CD19 binding domain comprises a 47G4 scFv. Examples of anti-CD19 CAR includes, without limitation, CAR1 and CAR2.

In certain embodiments, an anti-CD20 CAR comprises Leu16 anti-CD20 scFv, IgG4 hinge, CD28 transmembrane domain, 4-1BB co-stimulatory domain, and a CD3ζ signaling domain. In certain embodiments, an anti-CD20 CAR comprises Leu16 anti-CD20 scFv, IgG4 hinge, CD28 transmembrane and co-stimulatory domains, 4-1 BB co-stimulatory domain, and a CD3ζ signaling domain. In certain embodiments, an anti-CD20 CAR comprises 2.1.2 anti-CD20 scFv, CD28 hinge, transmembrane, and co-stimulatory domains, and a CD3ζ signaling domain. Examples of anti-CD20 CAR include, without limitation, CAR7, CAR22, and CAR25.

In certain embodiments, the cells comprising the mRNA disclosed herein, or cells comprising the polypeptide encoded by the mRNA, are prepared by transfecting the cells with the mRNA disclosed herein. In certain embodiments, the transfecting step comprises administering to the cells a pharmacologically effective dose of a pharmaceutical composition comprising the mRNA formulated in an LNP or tLNP as disclosed herein. In some embodiments, the pharmacologically effective dose is a therapeutically effective dose. In some embodiments, the transfecting takes place in vivo. In some embodiments, the transfecting takes place extracorporeally.

(V) LNP and tLNP Formulated Pharmaceutical Compositions

In certain other aspects, this disclosure provides a pharmaceutical composition comprising an mRNA formulated in an LNP, wherein the mRNA comprises an improved mRNA disclosed herein. The LNPs and tLNPs encapsulating the improved mRNAs disclosed herein are multicomponent compositions comprising, in addition to one or more mRNAs and also encapsulate additional nucleic acids (for example, a guide RNA), various lipids and, in the case of a tLNP, a targeting moiety. Additionally, the improved mRNAs themselves are assembled from multiple component nucleotide sequences. Each of these components is disclosed as multiple alternative species constituting genera and sub-genera. Each species, subgenus, and genus of any one component can be independently combined with species, subgenera, or genera of other components to define the scope of particular embodiments consistent with the teachings herein disclosed. Each of the components constitute means for accomplishing the function herein described. The complete LNPs and tLNPs themselves constitute means for transfecting a cell or means for delivering an nucleic acid (an RNA or mRNA) to a cell.

(V)(a) mRNA Payload

In some embodiments, an mRNA payload comprises one or more species of mRNA molecule. In some embodiments, the one or more species of mRNA comprises only a single species of mRNA while in other instances the one or more species of mRNA comprises multiple mRNA species, for example, 2, 3, or 4 mRNA species. In some embodiments in which the payload comprises multiple mRNA species, more than one of the species up to and including all of the species encode a polypeptide that is reactive with a same target. For example, in embodiments in which the payload comprises a nucleic acid encoding a CAR or immune cell engager (ICE), the payload can comprise or consist of 1) a single nucleic acid species encoding a single species of CAR or ICE, 2) a single nucleic acid species encoding 2 or more species of CAR or ICE (or a mixture of CAR and ICE) such as a bicistronic or multicistronic mRNA in which each CAR and/or ICE has specificity for a same target antigen, 3) a single nucleic acid species encoding 2 or more species of CAR or ICE (or a mixture of CAR and ICE) such as a bicistronic or multicistronic mRNA in which at least one CAR and/or ICE has specificity for a different target antigen than the other(s), 4) two or more nucleic acid species encoding 2 or more species of CAR or ICE (or a mixture of CAR and ICE) in which each CAR and/or ICE has specificity for a same target antigen, 5) two or more nucleic acid species encoding 2 or more species of CAR or ICE (or a mixture of CAR and ICE) in which at least one CAR and/or ICE has specificity for a different target antigen than the other(s). In some instances, a single species of mRNA is any one of the herein disclosed improved mRNA molecules. In other instances, a single species of mRNA has one of the particular combinations of UTRs disclosed herein. When two or more CAR and/or ICE have specificity for a same target antigen, they can have specificity for different epitopes of the same target antigen. Further variations will be apparent to one of skill in the art (e.g., multiple bi- or multicistronic nucleic acids, nucleic acids encoding a TCR and the like).

In other embodiments, an mRNA encodes a gene-editing or base-editing or gene writing protein. In some embodiments, a nucleic acid is a guide RNA. In some embodiments, an LNP or tLNP comprises both a gene- or base-editing or gene writing protein-encoding mRNA and one or more guide RNAs. CRISPR nucleases can have altered activity, for example, modifying the nuclease so that it is a nickase instead of making double-strand cuts or so that it binds the sequence specified by the guide RNA but has no enzymatic activity. Base-editing proteins are often fusion proteins comprising a deaminase domain and a sequence-specific DNA binding domain (such as an inactive CRISPR nuclease).

In some embodiments, the reprogramming agent comprises an immune receptor (for example, a chimeric antigen receptor or a T cell receptor) or an immune cell engager (for example, a bispecific T cell engager (BiTE), a bispecific killer cell engager (BiKE), a trispecific kill cell engager (TriKE), a dual affinity retargeting antibody (DART), a TRIDENT (linking two DART units or a DART unit and a Fab domain), a macrophage engager (e.g., BiME), an innate cell engager, and the like).

In some embodiments, the reprogramming agent encodes or is a gene/genome editing component. In some embodiments, the gene/genome editing component is a guide RNA for an RNA-directed nuclease or other nucleic acid editing enzyme, clustered regularly interspaced short palindromic repeat RNA (crisprRNA), a trans-activating clustered regularly interspaced short palindromic repeat RNA (tracrRNA). In some embodiments, the gene/genome editing component is a nucleic acid-encoded enzyme, such as RNA-guided nuclease, a gene or base editing protein, a zinc finger nuclease (ZFN), a transcription activator-like effector nuclease (TALEN), a meganuclease, a transposase, or a CRISPR nuclease (e.g., Cas9 or Cas12, etc.). In some embodiments, the gene/genome editing component is DNA to be inserted or that serves as a template in gene or genome editing for example a template for repair of a double-strand break.

### (V)(b) Binding Moiety

In certain embodiments, the LNP is a tLNP comprising a binding moiety serving as a targeting moiety that specifically binds to a cell surface protein. In certain embodiments, a LNP or tLNP comprises a binding moiety derived from an anti-CD40*^{‡} antibody, an anti-LRRC15^{†‡} antibody, an anti-CTSK antibody, an anti-ADAM12^{‡} antibody, an anti-CLDN6 antibody, an anti-CLDN 18.2 antibody, an anti-DLL3 antibody, an anti-IL13Rα2 antibody, an anti-ITGA11 antibody, an anti-FAP*^{†‡} antibody, an anti-NOX4 antibody, an anti-SGCD antibody, an anti-SYNDIG1 antibody, an anti-CDH11^{‡} antibody, an anti-PLPP4 antibody, an anti-SLC24A2 antibody, an anti-PDGFRB*^{‡} antibody, an anti-THY1^{‡} antibody, an anti-ANTXR1^{‡} antibody, an anti-GAS1 antibody, an anti-CALHM5 antibody, an anti-SDC1*^{‡} antibody, an anti-HER2*^{†‡} antibody, an anti-TROP2*^{†‡} antibody, an anti-MSLN*^{‡} antibody, an anti-Nectin4^{†‡} antibody, or an anti-MUC16*^{†‡} antibody. In further embodiments, a LNP (or tLNP) comprises a binding moiety specific for an immune cell antigen selected from CD1, CD2*^{†‡}, CD3*^{†‡}, CD4*^{†‡}, CD5^{†‡}, CD7^{†‡}, CD8^{†}, CD11b^{‡}, CD14^{†‡}, CD16, CD25^{†‡}, CD26*^{‡}, CD27*^{†‡}, CD28*^{†‡}, CD30*^{†‡}, CD32*, CD38*^{†‡}, CD39^{‡}, CD40*^{†‡}, CD40L (CD154)*^{†‡}, CD44*^{‡}, CD45^{†‡}, CD56^{†‡}, CD64*^{‡}, CD62^{†‡}, CD68, CD69^{‡}, CD73^{†‡}, CD80*^{‡}, CD83^{‡}, CD86*^{‡}, CD95^{‡}, CD103^{‡}, CD119^{‡}, CD126^{‡}, CD137 (41BB)^{†‡}, CD150^{‡}, CD153^{‡}, CD161^{‡}, CD166^{‡}, CD183 (CXCR3)^{‡}, CD183 (CXCR5)^{‡}, CD223 (LAG-3)*^{†‡}, CD254^{‡}, CD275^{‡}, CD45RA, CTLA-4*^{†}*^{†}, DEC205, OX40^{†}, PD-1*^{†‡}, GITR^{†}, TIM-3*^{†‡}, FasL*^{‡}, IL18R1, ICOS (CD278)^{‡}, leu-12, TCR^{†}, TLR1, TLR2^{†‡}, TLR3*^{‡}, TLR4^{†‡}, TLR6, TREM2^{‡}, NKG2D^{‡}, CCR, CCR1 (CD191)^{‡}, CCR2 (CD192)*^{†‡}, CCR4(CD194)*^{†‡}, CCR6(CD196)^{‡}, CCR7^{‡}, low affinity IL-2 receptort^{†‡}, IL-7 receptor^{‡}, IL-12 receptor^{‡}, IL-15 receptor^{‡}, IL-18 receptor^{‡}, and IL-21 receptor^{‡}. In further embodiments, a tLNP comprises a binding moiety specific for an HSC surface molecule selected from CD117^{†}, CD34*^{‡}, CD44*^{‡}, CD45^{†‡}, CD90 (Thy1)^{‡}, CD105^{‡}, CD133^{‡}, BMPR2^{‡}, and Sca-1; or specific for an MSC surface molecules selected from CD70*^{‡}, CD105^{‡}, CD73^{‡}, Stro-1^{‡}, SSEA-3^{‡}, SSEA-4^{‡}, CD271^{‡}, CD146*, GD2*^{†‡}, SUSD2, Stro-4, MSCA-1, CD56^{‡}, CD200*^{†‡}, PODXL^{‡}, CD13^{‡}, CD29*^{‡}, CD44*^{‡}, CD45^{†‡}, and CD10^{‡}. In various embodiments, a binding moiety is an antibody or antigen-binding portion thereof. (* indicates that exemplary antibodies with the indicated specificity from which a binding moiety could be derived can be found in U.S. Pat. No. 11,326,182B2 Table 9 or 10. ^{†} indicates that exemplary antibodies with the indicated specificity from which a binding moiety could be derived can be found in Wilkinson & Hale, 2022. Both references cited and incorporated by reference above. ^{‡} indicates that exemplary antibodies with the indicated specificity from which a binding moiety could be derived can be found in the Therapeutic Antibody Database (TABS) at tabs.craic.com). Other suitable antibodies can be found in Appendix A. A more extensive discussion of antibodies recognizing many of the individual antigens listed above can be found in WIPO Publication WO2024040195A1 and U.S. patent application Ser. No. 18/731,223 which are each incorporated by reference for all that they teach about antibodies and related molecules that can be used to provide binding moieties for tLNPs.

In some embodiments, a binding moiety of a tLNP comprises an antigen binding domain of an anti-CD8 antibody. In some embodiments, an anti-CD8 antibody is a human antibody. In some embodiments, an anti-CD8 antibody is a chimeric or humanized antibody, such as a chimeric or humanized mouse anti-human CD8 antibody. In some instances, an anti-CD8 antibody is a humanized form of a mouse antibody, such as RPA-T8 (also referred to herein as CT8). As expressed on cells in a mammal, CD8 is a dimer, commonly of two α chains or one each of an α and β chain. CT8 recognizes an epitope on the α chain (sometime referred to as CD8a or CD8α). CT8 and its humanized derivatives can bind to both the α₂ and αβ dimers. In some embodiments, a humanized antigen binding domain derived from CT8 comprises a heavy chain variable region (VH) comprising an amino acid sequence that has at least 90% identity with the amino acid sequence of SEQ ID NO: 188 or 206 wherein the VH comprises a heavy chain CDR1 (VH-CDR1) comprising the amino acid sequence RYTFTDYX₁LH (SEQ ID NO: 220) wherein X₁ is N, S, Q, or A, a VH-CDR2 comprising the amino acid sequence FIYPYX₁GGTG (SEQ ID NO: 221) or FIYPYX₂GGTG (SEQ ID NO: 222) wherein X₂ is N, Q, D, S, or A, and a VH-CDR3 having the amino acid sequence DHRYX₁EGVSFDY (SEQ ID NO: 223); and a light chain variable region (VL) comprising an amino acid sequence that has at least 90% identity with the amino acid sequence of SEQ ID NO: 194 or 212, wherein the VL comprises a CDR1 (VL-CDR1) comprising the amino acid sequence RASESVX₃GFGX₁SFMN wherein X₃ is D, E, S, or A (SEQ ID NO: 224), VL-CDR2 comprising the amino acid sequence LASX₂LES (SEQ ID NO: 225), and a VL-CDR3 having the amino acid sequence QQX₂X₂EX₃PYT (SEQ ID NO: 226). In some embodiments, the antigen binding domain comprises a VL region having the amino acid sequence of one of SEQ ID NOs: 195-197 or 213-215. In some embodiments, the antigen binding domain comprises a VH region having the amino acid sequence of one of SEQ ID NOs: 189-193, 202-205, or 207-211. In some embodiments, the antigen binding domain comprises a VL region having the amino acid sequence of SEQ ID NO: 196 and a VH region having the amino acid sequence of one of SEQ ID NOs: 190 or 202-205. In some embodiments, the antigen binding domain comprises:
(a) a VH comprising the amino acid sequence of SEQ ID NO: 189 and a VL comprising the amino acid sequence of SEQ ID NO: 195;
(b) a VH comprising the amino acid sequence of one of SEQ ID NO: 190-193, and a VL comprising the amino acid sequence of SEQ ID NO: 196; or
(c) a VH comprising the amino acid sequence of one of SEQ ID NO: 190-193, and a VL comprising the amino acid sequence of SEQ ID NO: 197;
In some embodiments, VH-CDR1 has the amino acid sequence RYTFTDYNLH (SEQ ID NO: 181). In some embodiments, VH-CDR2 has the amino acid sequence FIYPYNGGTG (SEQ ID NO: 182). In some embodiments, VH-CDR3 has the amino acid sequence DHRYNEGVSFDY (SEQ ID NO: 183). In some embodiments, VL-CDR1 has the amino acid sequence RASESVDGFGNSFMN (SEQ ID NO: 185). In some embodiments, VL-CDR2 has the amino acid sequence LASNLES (SEQ ID NO: 186). In some embodiments, VL-CDR3 has the amino acid sequence QQNNEDPYT (SEQ ID NO: 187). In some embodiments, the acceptor sequence from which the heavy chain framework regions are derived from IGHV1-46*01/IGHJ6*01, as shown in SEQ ID NO: 188). In some embodiments, the acceptor sequence from which the light chain framework regions are derived from IGKV1-39*01/IGKJ2*01, as shown in SEQ ID NO: 194. In some embodiments, the acceptor sequence from which the heavy chain framework regions are derived from a modified IGHV1-18*01, as shown in SEQ ID NO: 206. In some embodiments, the acceptor sequence from which the light chain framework regions are derived from a modified version of IGKV3D-1 1*01, as shown in SEQ ID NO: 212. In some embodiments, the heavy chain framework regions are derived from IGHV1-46*01/IGHJ6*01 and the light chain framework regions are derived from IGKV1-39*01/IGKJ2*01. The resultant VH and VL sequences are highly similar regardless of which acceptor sequence they are derived from. In some embodiments, the heavy chain framework regions are derived from a modified IGHV1-18*01 and the light chain framework regions are derived from a modified version of IGKV3D-11*01. In some embodiments, the heavy chain framework regions are derived from IGHV1-46*01/IGHJ6*01 and the light chain framework regions are derived from a modified version of IGKV3D-11*01. In some embodiments, the heavy chain framework regions are derived from a modified IGHV1-18*01 and the light chain framework regions are derived from IGKV1-39*01/IGKJ2*01. Table 17 shows several humanized variants of anti-CD8 antibody binding moiety and related sequences. Further information about humanized CT8 antibodies and antigen binding fragments thereof is disclosed in U.S. Provisional Patent Application 63/610,917 (Attorney docket number 23-1742-US-PRO) entitled Humanized Anti-CD8 Antibodies and Uses Thereof, filed on Dec. 15, 2023, which is incorporated by reference in its entirety for its teachings about such antibodies and antigen binding domains and their uses.

In some embodiments, the targeting moiety is a whole antibody. In some embodiments, the antibody comprises a silenced Fc region. A silenced Fc region fails to bind to Fc gamma receptors and complement protein C1q, thus abolishing immune effector functions. In some instances, the antibody comprises a silenced Fc region having the amino acid sequence of SEQ ID NO: 218 or 219. In some embodiments, the whole humanized anti-CD8 antibody heavy chain with a silenced Fc region comprises the sequence of CBD1033HC (SEQ ID NO: 347). In some embodiments, the whole humanized anti-CD8 antibody light chain comprises the sequence of CBD1033LC (SEQ ID NO: 348). In some embodiments, the whole humanized anti-CD8 antibody comprising a heavy chain with a silenced Fc region comprises a heavy chain comprising the sequence of CBD1033HC (SEQ ID NO: 347) and a light chain comprising the sequence of CBD1033LC (SEQ ID NO: 348). Natural Fc sequence ends with a lysine residue, as shown in SEQ ID NO: 219. In product manufacturing C-terminal clipping of this lysine residue can cause product heterogeneity. To obviate this problem the coding sequence for the Fc can be modified to not encode this amino acid, for example ending at the previous glycine residue in the natural Fc sequence as done in SEQ ID NO: 218. For all embodiments comprising an Fc, whether shown with or without the natural terminal lysine residue, there is an alternative embodiment without or with that residue, respectively.

### (V)(c) LNP Compositions

The LNP composition contributes to the formation of stable LNPs and tLNPs, efficient encapsulation of a payload, protection of a payload from degradation until it is delivered into a cell, and promotion of endosomal escape of a payload into the cytoplasm. These functions are primarily independent of the specificity of the binding moiety (or moieties) serving to direct or bias the tLNP to a particular cell type(s).

In certain embodiments the LNP comprises a lipid composition comprising an ionizable cationic lipid, PEG-lipid comprising functionalized PEG-lipid and non-functionalized PEG-lipid, a phospholipid, and a sterol.

The LNPs and/or tLNPs can include the various components in amounts sufficient to provide a nanoparticle with a desired shape, fluidity, and bio-acceptability as described herein. With respect to LNPs or tLNPs of this disclosure, in some embodiments, the LNP (or tLNP) comprises at least one ionizable cationic lipid (e.g., as described herein) in an amount in the range of from about 35 to about 65 mol %, or any integer bound sub-range thereof, e.g., in an amount of from about 40 to about 65 mol %, about 40 to about 60 mol %, or about 40 molt % to about 62 mol %. In some embodiments, the LNP or tLNP comprises about 58 mol %, about 60 mol %, or 62 mol % ionizable cationic lipid. In some embodiments, the LNP (or tLNP) comprises a phospholipid in an amount in the range of from about 7 to about 30 mol %, or any integer bound sub-range thereof, e.g., in an amount of from about 13 to about 30 mol %. In some embodiments, the LNP or tLNP comprises about 10 mol % phospholipid. In some embodiments, the LNP (or tLNP) comprises a sterol in an amount in the range of from about 20 to about 50 mol % or any integer bound sub-range thereof, e.g., in an amount in the range of from about 20 to about 45 mol %, or about 30 to about 50 mol %, or about 30 to about 45 mol %. In some embodiments, the LNP or tLNP comprises about 30.5, 26.5, or 23.5 mol % sterol. In some embodiments, the LNP (or tLNP) comprises at least one co-lipid in an amount in the range of from about 1 to about 30 mol %. In some embodiments, an LNP or tLNP comprises total PEG-lipid in an amount in the range of from about 1 mol % to about 5 mol % or any integer×10⁻¹ bound sub-range thereof, e.g., in an amount in the range of from about 1 mol % to about 2 mol % total PEG-lipid. In some embodiments, the LNP (or tLNP) comprises at least one unfunctionalized PEG-lipid in an amount of from 0 to about 5 mol % or any integer×10⁻¹ bound sub-range thereof, e.g., in the range of amount 0 to about 3 mol %, or about 1 to about 5 mol %, about 0.5 to about 5 mol %, or about 0.5 to about 3 mol %. In some embodiments, the LNP or tLNP comprises about 1.4 mol % unfunctionalized PEG-lipid. In some embodiments, the LNP or tLNP comprises at least one functionalized PEG-lipid in an amount in the range of from about 0.1 to about 5 mol % or any integer×10⁻¹ bound sub-range thereof, e.g., in the range of from about 0.1 to 0.3 mol %. In certain embodiments, an LNP or tLNP comprises about 0.1 mol %, about 0.2 mol %, or about 0.3 mol % functionalized PEG-lipid. In some embodiments, the LNP or tLNP comprises about 0.1 mol % functionalized PEG-lipid. In some embodiments, the functionalized PEG-lipid is conjugated to a binding moiety. In certain instances, a tLNP is an LNP that further comprises an antibody (for example, a whole IgG) as the binding moiety which is present at an antibody:mRNA ratio (w/w) of about 0.3 to about 1.0.

In certain aspects, this disclosure provides an LNP or tLNP, wherein the LNP or tLNP comprises about 35 mol % to about 65 mol % of an ionizable cationic lipid, about 0.5 mol % to about 3 mol % of a PEG-lipid (including non-functionalized PEG-lipid and optionally a functionalized PEG-lipid), about 7 mol % to about 13 mol % of a phospholipid, and about 30 mol % to about 50 mol % of a sterol. In some embodiments, an LNP or tLNP comprises a payload with a net negative charge for example, a peptide, a polypeptide, a protein, a small molecule, or a nucleic acid molecule, and combinations thereof. A payload is generally encompassed by or in the interior of an LNP or tLNP. As disclosed herein dosages always refer to the amount of payload being provided. In some embodiments, a payload comprises one or more species of nucleic acid molecule. For tLNP encapsulating mRNA dosages are typically in the range of 0.05 to 5 mg/kg without regard for recipient species. In some embodiments, the dosage is in the range of 0.1 to 1 mg/kg.

With respect to LNPs or tLNPs of this disclosure, in some embodiments, the ratio of total lipid to nucleic acid is about 10:1 to about 50:1 on a weight basis. In some embodiments, the ratio of total lipid to nucleic acid is about 10:1, about 20:1, about 30:1, or about 40:1 to about 50:1, or 10:1 to 20:1, 30:1, 40:1 or 50:1, or any range bound by a pair of these ratios. The ratio of lipid to nucleic acid can also be reported as an N/P ratio, the ratio of positively chargeable lipid amine (N=nitrogen) groups to negatively-charged nucleic acid molecule phosphate (P) groups. In some embodiments, the N/P ratio is from about 3 to about 9, about 3 to about 7, about 3 to about 6, about 4 to about 6, about 5 to about 6, or about 6. In some embodiments, the N/P ratio is from 3 to 9, 3 to 7, 3 to 6, 4 to 6, 5 to 6, or 6. In certain embodiments as described herein, the LNP (or tLNP) comprises a binding moiety, wherein the binding moiety comprises an antigen binding domain of an antibody and wherein the antibody is a whole antibody and the ratio of a lipid to nucleic acid is in the range of from about 0.3 to about 1.0 w/w.

Due to physiologic and manufacturing constraints LNP or tLNP, particles with a hydrodynamic diameter of about 50 to about 150 nm are desirable for in vivo use. Accordingly, in some embodiments, the LNP or tLNP has a hydrodynamic diameter of 50 to 150 nm and in some embodiments the hydrodynamic diameter is ≤120, ≤110, ≤100, or ≤90 nm. Uniformity of particle size is also desirable with a polydispersity index (PDI) of ≤0.2 (on a scale of 0 to 1) being acceptable. Both hydrodynamic diameter and polydispersity index are determined by dynamic light scattering (DLS). Particle diameter as assessed from cryo-transmission electron microscopy (Cryo-TEM) can be smaller than the DLS-determined value.

Particular compositions for precursors to tLNPs and tLNPs are disclosed in U.S. patent application Ser. No. 18/731,223 filed May 31, 2024, as well as PCT Application No. PCT/US23/72426 filed Aug. 17, 2023, each of which is incorporated by reference in its entirety. LNP and tLNP compositions can include those of Table 15. In various embodiments, N/P can be from 3 to 9 or any integer-bound sub-range in that range or about any integer in that range

### (V)(c)(1) Phospholipids

As described above, in various embodiments, the LNPs and tLNPs include a phospholipid. As would be understood by the person or ordinary skill in the art, phospholipids are amphiphilic molecules. Due to the amphiphilic nature of phospholipids, these molecules are known to form bilayers and by including them in the LNPs and tLNPs, as described herein, they can provide membrane formation, stability, and rigidity. As used herein, phospholipids include a hydrophilic head group, including a functionalized phosphate group, and two hydrophobic tail groups derived from fatty acids. For example, in various embodiments as described herein, the phospholipids include a phosphate group functionalized with ethanolamine, choline, glycerol, serine, or inositol. As described above, the phospholipid includes two hydrophobic tail groups derived from fatty acids. These hydrophobic tail groups can be derived from unsaturated or saturated fatty acids. For example, the hydrophobic tail groups can be derived from a C₁₂-C₂₀ fatty acid.

With respect to LNPs or tLNPs of this disclosure, in various embodiments, the phospholipid comprises dimyristoylphosphatidyl glycerol (DMPG), dimyristoylphosphatidyl choline (DMPC), dipalmitoyl phosphatidylcholine (DPPC), distearoylphosphatidylcholine (DSPC), distearoyl-glycero-phosphate (18:0 PA, DSGP), dioleoylphosphatidyl ethanolamine (DOPE), dioleoyl-glycero-phosphate (18:1 PA, DOGP), or diarachidoylphosphotidylcholine (DAPC), or a combination thereof. In various embodiments, the phospholipid is dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC). In some embodiments, the phospholipid is distearoylphosphatidylcholine (DSPC). Phospholipids can contribute to formation of a membrane, whether monolayer, bilayer, or multi-layer, surrounding the core of the LNP or tLNP. Additionally, phospholipids such as DSPC, DMPC, DPPC, DAPC impart stability and rigidity to membrane structure. Phospholipids, such as DOPE, impart fusogenicity. Further phospholipids, such as DMPG, which attains negative charge at physiologic pH, facilitates charge modulation. Thus, phospholipids constitute means for facilitating membrane formation, means for imparting membrane stability and rigidity, means for imparting fusogenicity, and means for charge modulation. Some embodiments specifically include one or more of the above phospholipids while other embodiments specifically exclude one or more of the above phospholipids.

In some embodiments, an LNP or tLNP has about 7 mol % to about 13 mol % phospholipid, about 7 mol % to about 10 mol % phospholipid, or about 10 mol % to about 13 mol % phospholipid. In certain embodiments, an LNP has about 7 mol %, about 10 mol %, or about 13 mol % phospholipid. In certain instances, the phospholipid is DSPC. In certain instances, the phospholipid is DAPC.

### (V)(c)(2) Sterols

The disclosed LNP and tLNP comprise a sterol. Sterol refers to a subgroup of steroids that contain at least one hydroxyl (OH) group. More specifically, a gonane derivative with an OH group substituted for an H at position 3, or said differently, but equivalently, a steroid with an OH group substituted for an H at position 3. Examples of sterols include, without limitation, cholesterol, ergosterol, β-sitosterol, stigmasterol, stigmastanol, 20-hydroxycholesterol, 22-hydroxycholesterol, and the like. With respect to LNPs or tLNPs of this disclosure, in various embodiments, the sterol is cholesterol, 20-hydroxycholesterol, 20(S)-hydroxycholesterol, 22-hydroxycholesterol, or a phytosterol or combinations thereof. In further embodiments, the phytosterol comprises campesterol, sitosterol, or stigmasterol, or combinations thereof. In certain embodiments, the cholesterol is not animal-sourced but is obtained by synthesis using a plant sterol as a starting point. LNPs incorporating C-24 alkyl (such as methyl or ethyl) phytosterols have been reported to provide enhanced gene transfection. The length of the alkyl tail, the flexibility of the sterol ring, and polarity related to a retained C-3 —OH group are important to obtaining high transfection efficiency. While β-sitosterol and stigmasterol performed well, vitamin D2, D3 and calcipotriol, (analogs lacking intact body of cholesterol) and betulin, lupeol ursolic acid and olenolic acid (comprising a 5th ring) should be avoided. Sterols serve to fill space between other lipids in the LNP or tLNP and influence LNP or tLNP shape. Sterols also control fluidity of lipid compositions, reducing temperature dependence. Thus, sterols such as cholesterol, ergosterol, 20-hydroxycholesterol, 22-hydroxycholesterol, campesterol, fucosterol, β-sitosterol, and stigmasterol constitute means for controlling LNP shape and fluidity or sterol means for increasing transfection efficiency. Some embodiments specifically include one or more of the above sterols while other embodiments specifically exclude one or more of the above sterols. In designing a lipid composition for a LNP or tLNP, in some embodiments, sterol content can be chosen to compensate for different amounts of other types of lipids, for example, ionizable cationic lipid or phospholipid.

In some embodiments, an LNP or tLNP has about 27 mol % or about 30 mol % to about 50 mol % sterol, or about 30 mol % to about 38 mol % sterol. In certain embodiments, an LNP or tLNP has about 30.5 mol %, about 33.5 mol %, or about 37.5 mol % sterol. In certain embodiments, an LNP or tLNP has 27 mol % or 30 mol % to 50 mol % sterol or 30 mol % to 38 mol % sterol. In further embodiments, an LNP or tLNP has 30.5 mol %, 33.5 mol %, or 37.5 mol % sterol. In certain instances, the sterol is cholesterol. In certain embodiments, the sterol is a mixture of sterols, for example, cholesterol and β-sitosterol or cholesterol and 20-hydroxycholesterol. In some instances, the sterol component is about 25 mol % 20-hydroxycholesterol and about 75 mol % cholesterol. In some instances, the sterol component is about 25 mol % β-sitosterol and about 75 mol % cholesterol. In some instances, the sterol component is about 50 mol % β-sitosterol and about 50 mol % cholesterol. In some instances, a sterol component is 25 mol % 20-hydroxycholesterol and 75 mol % cholesterol. In further instances, a sterol component is 25 mol % β-sitosterol and 75 mol % cholesterol. In still further instances, a sterol component is 50 mol % β-sitosterol and 50 mol % cholesterol.

### (V)(c)(3) Co-Lipids

With respect to the LNP or the tLNP, in some embodiments, the co-lipid is absent or comprises an ionizable lipid. In some embodiments the ionizable lipid is cholesterol hemisuccinate (CHEMS). In some embodiments, the co-lipid is a charged lipid, such as a quaternary ammonium headgroup-containing lipid. In some instances, the quaternary ammonium headgroup-containing lipid comprises 1,2-dioleoyl-3-trimethylammonium propane (DOTAP), N-(1-(2,3-dioleyloxy)propyl)-N,N,N-trimethylammonium chloride (DOTMA), or 3β-(N—(N′,N′-Dimethylaminoethane)carbamoyl)cholesterol (DC-Chol), or combinations thereof. In addition to the chloride salts of the quaternary ammonium headgroup containing lipids, further instances include bromide, mesylate, and tosylate salts.

### (V)(c)(4) PEG-Lipids

With respect to a LNP or tLNP of this disclosure, a PEG-lipid is a lipid conjugated to a polyethylene glycol (PEG). In some embodiments as described herein, the PEG-lipid is a C₁₄-C₂₀ lipid conjugated with a PEG. For example, in various embodiments as described herein, the PEG-lipid is a C₁₄-C₂₀ lipid conjugated with a PEG, or a C₁₄-C₁₈ lipid conjugated with a PEG, or a C₁₄-C₁₆ lipid conjugated with a PEG. In certain embodiments as described herein, the PEG-lipid is a fatty acid conjugated with a PEG. The fatty acid of the PEG-lipid can have a variety of chain lengths. For each, in some embodiments, the PEG-lipid is a fatty acid conjugated with PEG, wherein the fatty acid chain length is in the range of C₁₄-C₂₀ (e.g., in the range of C₁₄-C₁₈, or C₁₄-C₁₆). PEG-lipids with fatty acid chain lengths less than C₁₄ are too rapidly lost from the LNP or tLNP while those with chain lengths greater than C20 are prone to difficulties with formulation.

PEG can be made in a large range of sizes. In certain embodiments, the PEG of the disclosed LNP and tLNP is PEG-1000 to PEG-5000. It is to be understood that polyethylene preparations of these sizes are polydisperse and that the nominal size indicates an approximate average molecular weight of the distribution. Taking the molecular weight of an individual repeating unit of (OCH₂CH₂)ₙ to be 44, a PEG molecule with n=22 would have a molecular weight of 986, with n=45 a molecular weight of 1998, and with n=113 a molecular weight of 4990. n≈22 to 113 is used to represent PEG-lipids incorporating PEG moieties in the range of PEG-1000 to PEG-5000 such as PEG-1000, PEG-1500, PEG-2000, PEG-2500, PEG-3000, PEG-3500, PEG-4000, PEG-4500, and PEG-5000, although some molecules from preparations at the average molecular weight boundaries will have an n outside that range. For individual preparations n≈22 is used to represent PEG-lipids incorporating PEG moieties from PEG-1000, n≈45 is used to represent PEG-lipids incorporating PEG moieties from PEG-2000 n≈67 is used to represent PEG-lipids incorporating PEG moieties from PEG-3000, n≈90 is used to represent PEG-lipids incorporating PEG moieties from PEG-4000, n≈113 is used to represent PEG-lipids incorporating PEG moieties from PEG-5000. Some embodiments incorporate PEG moieties in a range bounded by any pair of the foregoing values of n or average molecular weight. In some embodiments of the PEG-lipid, a PEG is of 500-5000 or 1000-5000 Da molecular weight (MW). For example, in some embodiments, the PEG of the PEG-lipid has a molecular weight in the range of 1500-5000 Da or 2000-5000 Da. In some embodiments as described herein, the PEG-lipid has a molecular weight in the range of 500-4000 Da, or 500-3000 Da, or 1000-4000 Da, or 1000-3000, or 1000-2500, or 1500-4000, or 1500-3000, or 1500-2500 Da. In some embodiments, the PEG moiety is PEG-500, PEG-1000, PEG-1500, PEG-2000, PEG-2500, PEG-3000, PEG-3500, PEG-4000, PEG-4500, and PEG-5000. In some embodiments, the PEG unit has a MW of 2000 Da (sometime abbreviated as PEG(2k)). Some embodiments incorporate PEG moieties of PEG-1000, PEG-2000, or PEG-5000. In some instances, the PEG moiety is PEG-2000. Certain embodiments comprise a DSG-PEG, for example, DSG-PEG-2000. Certain embodiments comprise a DSPE-PEG, for example, DSPE-PEG-2000. Certain embodiments comprise both DSG-PEG-2000 and/or DSPE-PEG2000.

Common PEG-lipids fall into two classes diacyl glycerols and diacyl phospholipids. Examples of diacyl glycerol PEG-lipids include DMG-PEG (1,2-dimyristoyl-glycero-3-methoxypolyethylene glycol), DPG-PEG (1,2-dipalmitoyl-glycero-3-methoxypolyethylene glycol), DSG-PEG (1,2-distearoyl-glycero-3-methoxypolyethylene glycol), and DOG-PEG (1,2-dioleoyl-glycero-3-methoxypolyethylene glycol). Examples of diacyl phospholipids include DMPE-PEG (1,2-dimyristoyl-glycero-3-phosphoethanolamine-3-methoxypolyethylene glycol), DPPE-PEG (1,2-dipalmitoyl-glycero-3-phosphoethanolamine-3-methoxypolyethylene glycol), DSPE-PEG (1,2-distearoyl-glycero-3-phosphoethanolamine-3-methoxypolyethylene glycol), and DOPE-PEG (1,2-dioleoyl-glycero-3-phosphoethanolamine-3-methoxypolyethylene glycol).

In some embodiments, the MW2000 PEG-lipid (e.g., a PEG-lipid comprising a PEG of a molecular weight of 2000 Da) comprises DMG-PEG2000 (1,2-dimyristoyl-glycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof. In some embodiments, the PEG unit has a MW of 2000 Da. In some embodiments, the MW2000 PEG-lipid comprises DMrG-PEG2000 (1,2-dimyristoyl-rac-glycero-3-methoxypolyethylene glycol-2000), DPrG-PEG2000 (1,2-dipalmitoyl-rac-glycero-3-methoxypolyethylene glycol-2000), DSrG-PEG2000 (1,2-distearoyl-rac-glycero-3-methoxypolyethylene glycol-2000), DorG-PEG2000 (1,2-dioleoyl-glycero-3-methoxypolyethylene-rac-glycol-2000), DMPEr-PEG200 (1,2-dimyristoyl-rac-glycero-3-phosphoethanolamine-3-methoxypolyethylene glycol-2000), DPPEr-PEG2000 (1,2-dipalmitoyl-rac-glycero-3-phosphoethanolamine-3-methoxypolyethylene glycol-2000), DSPEr-PEG2000 (1,2-distearoyl-rac-glycero-3-phosphoethanolamine-3-methoxypolyethylene glycol-2000), DOPEr-PEG2000 (1,2-dioleoyl-rac-glycero-3-phosphoethanolamine-3-methoxypolyethylene glycol-2000), or combinations thereof. The glycerol in these lipids is chiral. Thus, in some embodiments, the PEG-lipid is racemic. Alternatively, optically pure antipodes of the glycerol portion can be employed, that is, the glycerol portion is homochiral. As used herein with respect to glycerol moieties, optically pure means≥95% of a single enantiomer (D or L). In some embodiments, the enantiomeric excess is ≥98%. In some embodiments, the enantiomeric excess is ≥99%. Additional PEG-lipids, including achiral PEG-lipids built on a symmetric dihydroxyacetone scaffold, a symmetric 2-(hydroxymethyl)butane-1,4-diol, or a symmetric glycerol scaffold, are disclosed in U.S. Provisional Application No. 63/362,502, filed on Apr. 5, 2022, and PCT/US2023/017648 application filed on Apr. 5, 2023 (WO 2023/196445), both entitled PEG-Lipids and Lipid Nanoparticles, which are incorporated by reference in their entirety.

The above PEG-lipid examples are presented as methoxypolyethylene glycols, but the terminus need not necessarily be methoxyl. With respect to any of the PEG-lipids that have not been functionalized, in alternative embodiments, the PEG moiety of the PEG lipids can terminate with a methoxyl, a benzyloxyl, a 4-methoxybenzyloxyl, or a hydroxyl group (that is, an alcohol). The terminal hydroxyl facilitates functionalization. The methoxyl, benzyloxyl, and 4-methoxybenzyloxyl groups are advantageously provided for PEG-lipid that will be used as a component of the LNP without functionalization. However, all four of these alternatives are useful as the (non-functionalized) PEG-lipid component of LNPs. The 4-methoxybenzyloxyl group, often used as a protecting group during synthesis of the PEG-lipid, is readily removed to generate the corresponding hydroxyl group. Thus, the 4-methoxybenzyloxyl group offers a convenient path to the alcohol when it is not synthesized directly. The alcohol is useful for being functionalized, prior to incorporation of the PEG-lipid into a LNP, so that a binding moiety can be conjugated to it as a targeting moiety for the LNP (making it a tLNP). As used herein, the terminus of the PEG moiety, and similar constructions, refers to the end of the PEG moiety that is not attached to the lipid.

A PEG-moiety provides a hydrophilic surface on the LNP, inhibiting aggregation or merging of LNP, thus contributing to their stability and reducing polydispersity, i.e. reducing the heterogeneity of a dispersion of LNPs. Additionally, a PEG moiety can impede binding by the LNP, including binding to plasma proteins. These plasma proteins include apoE which is understood to mediate uptake of LNP by the liver so that inhibition of binding can lead to an increase in the proportion of LNP reaching other tissues. These plasma proteins also include opsonins so that inhibition of binding reduces recognition by the reticuloendothelial system. The PEG-moiety can also be functionalized to serve as an attachment point for a targeting moiety. Conjugating a cell- or tissue-specific binding moiety to the PEG-moiety enables a tLNP to avoid the liver and bind to its target tissue or cell type, greatly increasing the proportion of LNP that reaches the targeted tissue or cell type. PEG-lipid can thus serve as means for inhibiting LNP binding, and PEG-lipid conjugated to a binding moiety can serve as means for LNP-targeting.

As used herein, the term “functionalized PEG-lipid” and similar constructions refer generally to both the unreacted and reacted entities. The lipid composition of a LNP can be described referencing the reactive species even after conjugation has taken place (forming a tLNP). For example, a lipid composition can be described as comprising DSPE-PEG-maleimide and can be said to further comprise a binding moiety without explicitly noting that upon reaction to form the conjugate the maleimide will have been converted to a succinimide (or hydrolyzed succinimide). Similarly, if the reactive group is bromomaleimide, after conjugation it will be maleimide. These differences of chemical nomenclature for the unreacted and reacted species are to be implicitly understood even when not explicitly stated. Certain embodiments comprise a DSG-PEG, for example, DSG-PEG-2000. Certain embodiments comprise a functionalized DSPE-PEG, for example, functionalized DSPE-PEG-2000. Certain embodiments comprise both DSG-PEG-2000 and functionalized DSPE-PEG-2000. In some instances, the functionalized PEG-lipid is functionalized with a maleimide moiety, for example, DSPE-PEG-2000-MAL.

In certain aspects, the LNP comprises one or more PEG-lipids and/or functionalized PEG-lipids; when both a functionalized and unfunctionalized PEG-lipid, the PEG-lipid present they can be the same or different; and one or more ionizable cationic lipids; the LNP can further comprise a phospholipid, a sterol, a co-lipid, or any combination thereof. The term “functionalized PEG-lipid” refers to a PEG-lipid in which the PEG moiety has been derivatized with a chemically reactive group that can be used for conjugating a targeting moiety to the PEG-lipid. The functionalized PEG-lipid can be reacted with a binding moiety so that the binding moiety is conjugated to the PEG portion of the lipid. The conjugated binding moiety can thus serve as a targeting moiety for the LNP to constitute a tLNP. In some embodiments, the binding moiety is conjugated to the functionalized PEG-lipid after an LNP comprising the functionalized PEG-lipid is formed. In other embodiments, the binding moiety is conjugated to the PEG-lipid and then the conjugate is inserted into a previously formed LNP.

In certain embodiments, the LNP is a tLNP comprising one or more functionalized PEG-lipids that has been conjugated to a binding moiety. In certain embodiments, the tLNP also comprises PEG-lipids not functionalized or conjugated with a binding moiety. In some embodiments, the functionalization is a maleimide. In some embodiments the functionalization is a bromomaleimide or bromomaleimide amide, alkynylamide, or alkynylimide moiety at the terminal hydroxyl end of the PEG moiety. In some embodiments, the binding moiety comprises an antibody or antigen binding portion thereof. In some embodiments, the binding moiety is a polypeptide comprising a binding domain and an N- or C-terminal extension comprising an accessible thiol group. In some embodiments, the conjugation linkage comprises a reaction product of a thiol in the binding moiety with a functionalized PEG-lipid. In some embodiments, the functionalization is a maleimide, azide, alkyne, dibenzocyclooctyne (DBCO), bromomaleimide or bromomaleimide amide, alkynylamide, or alkynylimide. In some embodiments, the binding moiety comprises an antibody or antigen binding portion thereof. In some embodiments, the binding moiety is a polypeptide comprising a binding domain and an N- or C-terminal extension comprising an accessible thiol group.

In certain embodiments, the PEG-lipid and/or functionalized PEG-lipid comprises a scaffold selected from Formula S1, Formula S2, Formula S3, or Formula S4:

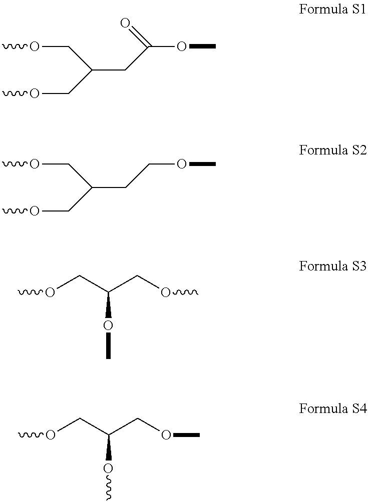

wherein [symbol] represents the points of ester connection with a fatty acid, and [symbol] represents the point of ester (S1) or ether (S2, S3, and S4) formation with the PEG moiety. In some embodiments, the fatty acid esters are C₁₄-C₂₀ straight-chain alkyl fatty acids. In some embodiments, the PEG moiety is functionalized and the fatty acid esters are C₁₆-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. By symmetric it is meant that each alkyl branch has the same number of carbons. In some embodiments, the branch is at the 3, 4, 5, 6, or 7 position of the fatty acid ester. The synthesis and use of PEG-lipids built on scaffolds S1-S4 is disclosed in WO2023/196445A1 which is incorporated by reference for all that it teaches about PEG-lipids and their use.

Some embodiments of the disclosed ionizable cationic lipids have head groups with small (<250 Da) PEG moieties. These lipids are not what is meant by the term PEG-lipid as used herein. These small PEG moieties are generally too small to impede binding to a similar extent as the larger PEG moieties of the PEG-lipids disclosed above, though they will impact the lipophilicity of ionizable cationic lipid. Moreover, the PEG-lipids are understood to be primarily located in an exterior facing lamella whereas much of the ionizable cationic lipid is in the interior of the LNP.

In certain embodiments, a functionalized PEG-lipid of a LNP or tLNP or this disclosure comprises one or more fatty acid tails, each that is no shorter than C₁₆ nor longer than C₂₀ for straight-chain fatty acids. For branched chain fatty acids, tails no shorter than C₁₄ fatty acids nor longer than C₂₀ are acceptable. In some embodiments, fatty acid tails are C₁₆. In some embodiments, the fatty acid tails are C₁₈. In some embodiments, the functionalized PEG-lipid comprises a dipalmitoyl lipid. In some embodiments, the functionalized PEG-lipid comprises a distearoyl lipid. The fatty acid tails serve as means to anchor the PEG-lipid in the tLNP to reduce or eliminate shedding of the PEG-lipid from the tLNP. This is a useful property for the PEG-lipid whether or not it is functionalized but has greater significance for the functionalized PEG-lipid as it will have a targeting moiety attached to it and the targeting function could be impaired if the PEG-lipid (with the conjugated binding moiety, such as an antibody) were shed from the tLNP.

In some embodiments, an LNP or tLNP comprises about 0.5 mol % to about 3 mol % or 0.5 mol % to 3 mol % PEG-lipid comprising functionalized and non-functionalized PEG-lipid. In certain embodiments, an LNP or tLNP comprises DSG-PEG. In other embodiments, an LNP or tLNP comprises DMG-PEG or DPG-PEG. In certain embodiments, an LNP or tLNP comprises DSPE-PEG. In some embodiments, the functionalized and non-functionalized PEG-lipids are not the same PEG-lipid, for example, the non-functionalized PEG-lipid can be a diacylglycerol and the functionalized PEG-lipid a diacyl phospholipid. tLNP with such mixtures have reduced expression in the liver, possibly due to reduced uptake. In certain embodiments the functionalized PEG-lipid is DSPE-PEG and the non-functionalized PEG-lipid is DSG-PEG. In some embodiments, an LNP or tLNP comprises about 0.4 mol % to about 2.9 mol % or about 0.9 mol % to about 1.4 mol % non-functionalized PEG lipid. In certain embodiments, an LNP or tLNP comprises about 1.4 mol % or 1.4 mol % non-functionalized PEG lipid. In some embodiments, an LNP or tLNP comprises about 0.1 mol % to about 0.3 mol % or 0.1 mol % to 0.3 mol % functionalized lipid. In some instances, the functionalized lipid is DSPE-PEG. In certain instances, an LNP or tLNP comprises about 0.1 mol %, about 0.2 mol %, or about 0.3 mol % DSPE-PEG. In certain instances, an LNP or tLNP comprises 0.1 mol %, 0.2 mol %, or 0.3 mol % DSPE-PEG. In certain instances, the functionalized PEG-lipid is conjugated to a binding moiety. As used herein, the phrase “is conjugated to” and similar constructions are meant to convey a state of being, that is, a structure, and not a process, unless context dictates otherwise.

### (V)(c)(5) Conjugation

Any suitable chemistry can be used to conjugate the binding moiety to the PEG of the PEG-lipid, including maleimide (see Parhiz et al., Journal of Controlled Release 291:106-115, 2018) and click (see Kolb et al., Angewandte Chemie International Edition 40(11):2004-2021, 2001; and Evans, Australian Journal of Chemistry 60(6):384-395, 2007) chemistries. Reagents for such reactions include lipid-PEG-maleimide, lipid-PEG-cysteine, lipid-PEG-alkyne, lipid, PEG-dibenzocyclooctyne (DBCO), and lipid-PEG-azide. Further conjugations reactions make use of lipid-PEG-bromo maleimide, lipid-PEG-alkylnoic amide, PEG-alkynoic imide, and lipid-PEG-alkyne reactions, as disclosed in PCT/US23/17648 entitled PEG-Lipids and Lipid Nanoparticles, which is incorporated by reference for all that it teaches about conjugation chemistry and alternative PEG-lipids. On the binding moiety side of the reaction one can use an existing cysteine sulfhydryl, or derivatize the protein by adding a sulfur containing carboxylic acid, for example, to the epsilon amino of a lysine to react with maleimide, bromomaleimide, (collectively, “a maleimide”), alkylnoic amide, or alkynoic imide. Alternatively, one can add an alkyne to a sulfhydryl or an epsilon amino of a lysine to participate in a click chemistry reaction.

To modify an epsilon amino of a binding moiety lysine to react with a maleimide functionalized PEG-lipid the binding moiety (e.g., an antibody) can be reacted with N-succinimidyl S-acetylthioacetate (SATA). SATA is then deprotected, for example, using 0.5 M hydroxylamine followed by removal of the unreacted components by G-25 Sephadex Quick Spin Protein columns (Roche Applied Science, Indianapolis, IN). The reactive sulfhydryl group on the binding moiety is then conjugated to maleimide moieties on LNPs of the disclosure using thioether conjugation chemistry. Purification can be performed using Sepharose CL-4B gel filtration columns (Sigma-Aldrich). tLNPs (LNPs conjugated with a targeting antibody) can be stored frozen at −80° C. until needed. Others have conjugated antibody to free functionalized PEG-lipid and then incorporated the conjugated lipid into pre-formed LNP. However, it was found that this procedure is more controllable and produces more consistent results.

There are also several approaches to site-specific conjugation. Particularly but not exclusively suitable for truncated forms of antibody, C-terminal extensions of native or artificial sequences containing a particularly accessible cysteine residue are commonly used. Partial reduction of cystine bonds in an antibody, for example, with tris(2-carboxy)phosphine (TCEP), can also generate thiol groups for conjugation which can be site-specific under defined conditions with an amenable antibody fragment. Alternatively, the C-terminal extension can contain a sortase A substrate sequence, LPXTG (SEQ ID NO: 349) which can then be functionalized in a reaction catalyzed by sortase A and conjugated to the PEG-lipid, including through click chemistry reactions (see, for example, Moliner-Morro et al., Biomolecules 10(12):1661, 2020 which is incorporated by reference herein for all that it teaches about antibody conjugations mediated by the sortase A reaction and/or click chemistry). The use of click chemistry for the conjugation of a targeting moiety, such as various forms of antibody, is disclosed, for example, in WO2024/102,770 which is incorporated by reference in its entirety for all that it teaches about the conjugation of targeting moieties to LNPs that is not inconsistent with this disclosure.

For whole antibody and other forms comprising an Fc region, site-specific conjugation to either (or both) of two specific lysine residues (Lys248 and Lys288) can be accomplished without any change to or extension of the native antibody sequence by use of one of the AJICAP® reagents (see, for example, Matsuda et al., Molecular Pharmaceutics 18:4058-4066, 2021 and Fujii et al., Bioconjugate Chemistry https://doi.org/10.1021/acs.bioconjchem.3c00040, 2023, which are incorporated by reference herein for all that they teach about conjugation of antibodies with AJICAP reagents). The AJICAP reagents are modified affinity peptides that bind to specific loci on the Fc and react with an adjacent lysine residue. The peptide is then cleaved with base to leave behind a thiol-functionalized lysine residue which can then undergo conjugation through maleimide or haloamide reactions, for example). Functionalization with azide or dibenzocyclooctyne (DBCO) for conjugation by click chemistry is also possible. This and similar technology are further described in US 2020/0190165 (corresponding to WO 2018/199337), US 2021/0139549 (corresponding to WO 2019/240287) and US 2023/0248842 (corresponding to WO 2020/184944) which are incorporated by reference in their entirety for all that they teach about such modified affinity peptides and their use.

Accordingly, in some embodiments the binding moiety is conjugated to the PEG moiety of the PEG-lipid through a thiol modified lysine residue. In some embodiments, the conjugation is through a cysteine residue in a native or added antibody sequence. In some embodiments, a particular cysteine residue is preferentially or exclusively reacted, for example, a cysteine residue in an antibody hinge region. In further instances, a binding moiety with a conjugatable cysteine residue in an antibody hinge region is an Fab′ or similar fragment. In other embodiments, the conjugation is through a sortase A substrate sequence. In still other embodiments, the conjugation is through a specific lysine residue (Lys248 or Lys288) in the Fc region.

### (V)(c)(6) Ionizable Cationic Lipids

Ionizable cationic lipids are useful components for complexing with negatively charged payloads and for promoting delivery of the payload into the cytoplasm of a cell following endocytosis. Accordingly, each of the hereinbelow disclosed genera and species of ionizable cationic lipid can be used in defining the scope of embodiments of the herein disclosed LNP and tLNP compositions and pharmaceutical compositions, and methods of using them. In certain embodiments, ionizable cationic lipid(s) of an LNP having a measured pKa of 6 to 7 can remain essentially neutral in the blood stream and interstitial spaces but ionize after uptake into cells as the endosomes acidify. Upon acidification in the endosomal space, the lipid becomes protonated, and associates more strongly with the phosphate backbone of the nucleic acid, which destabilizes the structure of the LNP and promotes nucleic acid release from the LNP into the cell cytoplasm (also referred to as endosomal escape). Thus, the herein disclosed ionizable cationic lipids constitute means for destabilizing LNP structure (when ionized) or means for promoting nucleic acid release or endosomal escape. In some embodiments the ionizable cationic lipid has a c-pKa from 8 to 11 and cLogD from 9 to 18 or 11-14. In some embodiments, the ionizable cationic lipids have branched structure to give the lipid a conical rather than cylindrical shape. Suitable ionizable cationic lipids are known to those of skill in the art. In some embodiments, the ionizable cationic lipid has a structure of Formula 1, Formula 2, Formula 3, Formula M5, Formula M1, CICL, CICL-IE, Formula M6 or Formula M2 including species or subgenera thereof, as disclosed in International Application Number PCT/US2023/017647 published as WO 2023/196444, and U.S. Patent Application Nos. 63/632,931, 63/632,937, 63/632,940, and 63/632,944, also further describing the use of ionizable cationic lipids and LNP incorporating them, which is incorporated by reference in its entirety.

For simplicity, chemical moieties are defined and referred to throughout primarily as univalent chemical moieties (e.g., alkyl, aryl, and the like). Nevertheless, such terms can also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for nitrogen, 2 for oxygen, and 2, 4, or 6 for sulfur, depending on the oxidation state of the sulfur atom).

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups.

In some embodiments, the hydrocarbon chain is unsubstituted. In other embodiments, one or more hydrogens of the alkyl or alkenyl group can be substituted with the same or different substituents.

Aryl refers to an aromatic or heteroaromatic ring lacking one hydrogen leaving a bond that connects to another portion of an organic molecule. Examples of aryl include, without limitation, phenyl, naphthalenyl, pyridine, pyrimidine, pyrazine, pyrrole, furan, thiophene, imidazole, thiazole, oxazole, and the like.

Aryl-alkyl refers to a moiety comprising one or more aryl rings and one or more alkyl moieties. The position of the one or more aryl rings can vary within the alkyl portion of the moiety. For example, the one or more aryl rings can be at an end of the one or more alkyl moieties, be fused into the carbon chain of the one or more alkyl moieties, or substitute one or more hydrogens of one or more alkyl moieties; and the one or more alkyl moieties can substitute one or more hydrogens of the one or more aryl rings. In some embodiments, there is a single ring; while in other embodiments, that are multiple rings.

Branched alkyl is a saturated alkyl moiety wherein the alkyl group is not a straight chain. Alkyl portions such as methyl, ethyl, propyl, butyl, and the like, can be appended to variable positions of the main alkyl chain. In some embodiments, there is a single branch; while in other embodiments, there are multiple branches.

Branched alkenyl refers to an alkenyl group comprising at least one branch off the main chain which can be formed by substituting one or more hydrogens of the main chain with the same or different alkyl groups, e.g., without limitation, methyl, ethyl, propyl, butyl, and the like. In some embodiments, a branched alkenyl is a single branch structure, while in other embodiments, a branched alkenyl can have multiple branches.

Straight chain alkyl is a non-branched, non-cyclic version of the alkyl moiety described above.

Straight chain alkenyl is a non-branched, non-cyclic version of the alkenyl moiety described above.

In some embodiments as described herein, the ionizable cationic lipids have a structure of Formula 1:

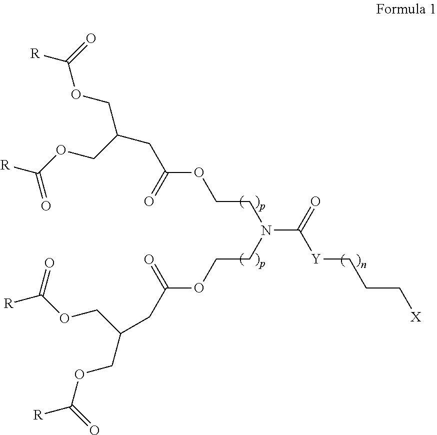

wherein:
Y is O, NH, N—OH₃, or OH₂,
n is an integer from 0 to 4,
X is

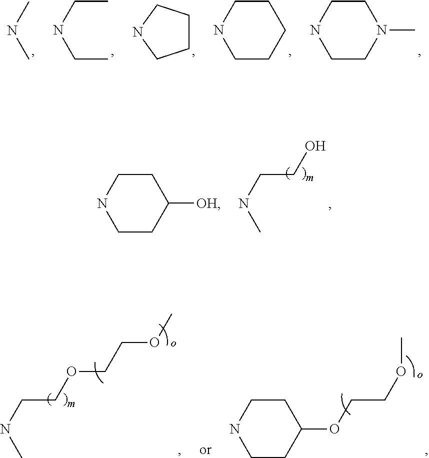

m is an integer from 1 to 3,
o is an integer from 1 to 4,
p is an integer from 1 to 4,
wherein when p=1:
   each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain;
wherein when p=2:
   each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain;
wherein when p=3:
   each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain; and
wherein when p=4:
   each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

In some embodiments as described herein, the ionizable cationic lipids have a structure of Formula 2:

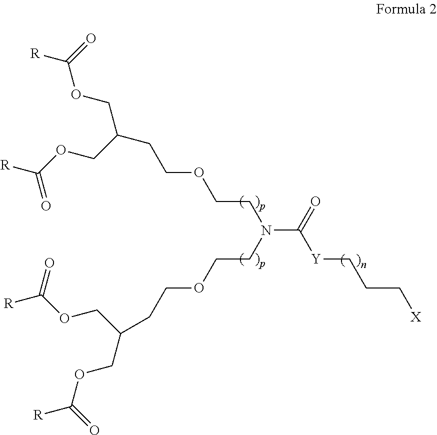

wherein:
Y is O, NH, N—OH₃, or OH₂,
n is an integer from 0 to 4,

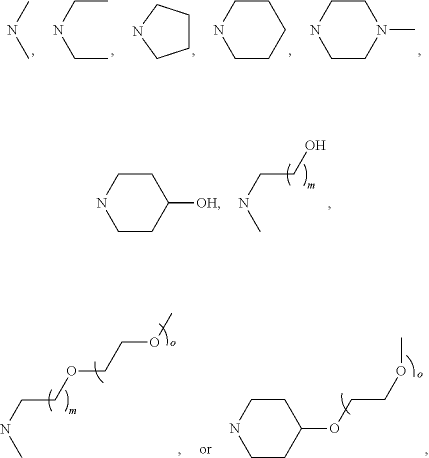

m is an integer from 1 to 3,
is an integer from 1 to 4,
p is an integer from 1 to 4,
wherein when p=1:
   each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2:
   each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain,
wherein when p=3:
   each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain; and
wherein when p=4:
   each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

In some embodiments as described herein, the ionizable cationic lipids have a structure of Formula 3:

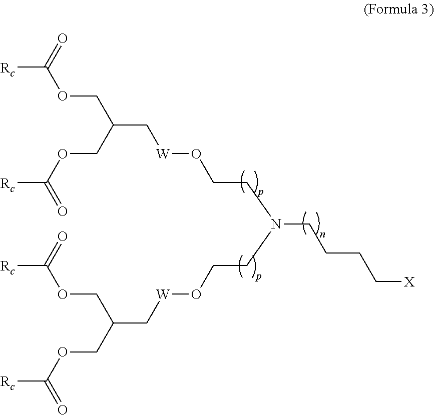

wherein:
W is C═O or CH₂,
n is an integer from 0 to 4,
X is

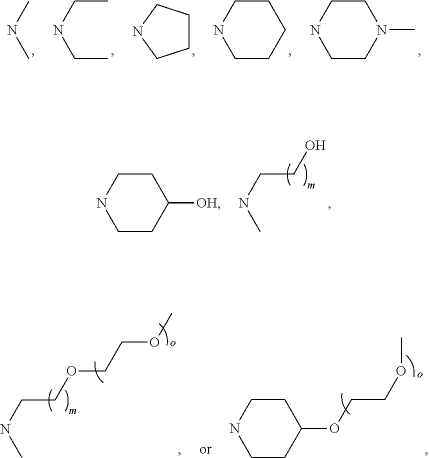

m is an integer from 1 to 3,
is an integer from 1 to 4,
p is an integer from 1 to 4,
wherein when p=1:
   each R^{c} is independently C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈ cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
wherein when p=2:
   each R^{c} is independently C₈ to C₁₆ straight-chain alkyl; C₈ to C₁₆ straight-chain alkenyl; C₈ to C₁₆ branched alkyl; C₈ to C₁₆ branched alkenyl; C₁₁ to C₁₆ cycloalkyl-alkyl in which the cycloalkyl is C₃ to C₈ cycloalkyl positioned at the either end or within the alkyl chain; or C₁₀ to C₁₈ aryl-alkyl in which the aryl is phenyl or naphthalenyl and is positioned at either end or within the alkyl chain,
wherein when p=3:
   each R^{c} is independently C₈ to C₁₄ straight-chain alkyl; C₈ to C₁₄ straight-chain alkenyl; C₈ to C₁₄ branched alkyl; C₈ to C₁₄ branched alkenyl; C₁₁ to C₁₄ cycloalkyl-alkyl in which the cycloalkyl is C₃ to C₈ cycloalkyl positioned at either end or within the alkyl chain; or C₁₀ to C₁₆ aryl-alkyl in which the aryl is phenyl or naphthalenyl and is positioned at the either end or within the alkyl chain, and
wherein when p=4:
   each R^{c} is independently C₈ to C₁₂ straight-chain alkyl; C₈ to C₁₂ straight-chain alkenyl; C₈ to C₁₂ branched alkyl; C₈ to C₁₂ branched alkenyl; C₁₁ to C₁₂ cycloalkyl-alkyl in which the cycloalkyl is C₃ to C₈ cycloalkyl positioned at either end or within the alkyl; or C₁₀ to C₁₄ aryl-alky in which the aryl is phenyl or naphthalenyl and is positioned at the either end or within the alkyl chain.
   R^{o} in the text and R_{c} in the chemical structure are equivalent.

In certain aspects, the ionizable cationic lipids of this disclosure have a structure of the formula M5:

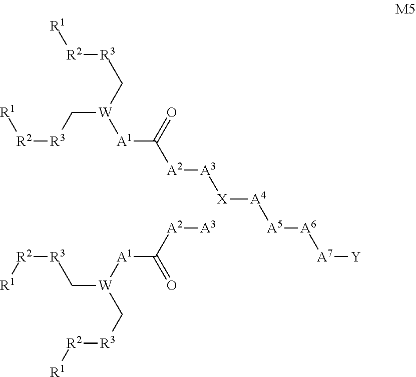

wherein:
each R¹ is independently selected from a C₇-C₁₁ alkyl or a C₇-C₁₁ alkenyl,
A¹ is (CH₂)₁₋₂,
A² is O,
A³ is (CH₂)₁₋₅, wherein A³ is not CH₂ if X is N,
X is N, CH, or C—CH₃,
A⁴ is CH₂, C═O, NH, NCH₃, or O,
A⁵ is absent, O, S, NH, or NCH₃ if A⁴ is C═O, or A⁵ is C═O if A⁴ is not C═O,
A⁶ is O, S, NH, NCH₃ or (CH₂)₀₋₂,
A⁷ is (CH₂)₀₋₆, wherein if A⁶ is O, S, NH, NCH₃, A⁷ is (CH₂)₂₋₄,
Y is

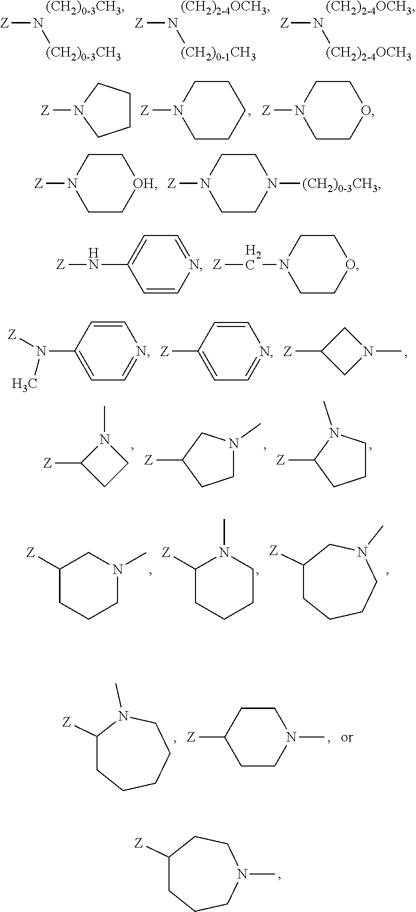

wherein Z is a bond; and
R² is O, R³ is C═O and W is CH or N, or R² is C═O, R³ is O and W is CH;
   wherein A⁶ and A⁷ are not both (CH₂)₀ unless A⁵ is C═O;
   wherein
a), A¹ is CH₂, A³ is (CH₂)₂₋₅, X is N, A⁴ is C═O, A⁵ is O, S, NH, NCH₃, A⁶ is (CH₂)₁₋₂, A⁷ is (CH₂)₁₋₄, or
b) A¹ is CH₂, A³ is (CH₂)₁₋₄, X is CH, A⁴ is CH₂, NH, NCH₃, O, A⁵ is C═O, A⁶ is O, NH, NCH₃, A⁷ is (CH)₂₋₆, or
c) A¹ is (CH₂)₂, A³ is (CH₂)₁₋₄, X is C—CH₃, A⁴ is C═O, A⁵ is O, NH, NCH₃, A⁶ is (CH₂)₁₋₂, A⁷ is (CH₂)₁₋₄, or
d) A¹ is CH₂, A³ is (CH₂)₂₋₅, X is N, A⁴ is C═O, A⁵ is absent, A⁶ is (CH₂)₀, A⁷ is (CH₂)₀, and Y is

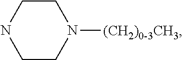

or
e) A¹ is CH₂, A³ is (CH₂)₁₋₅, X is CH, A⁴ is CH₂, NH, NCH₃ or O, A⁵ is C═O, A⁶ is (CH₂)₀, A⁷ is (CH₂)₀, and Y is

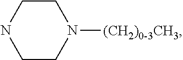

f) A¹ is (CH₂)₂, A³ is (CH₂)₁₋₅, X is CCH₃, A⁴ is C═O, A⁵ is absent, A⁶ is (CH₂)₀,
A⁷ is (CH₂)₀, and Y is

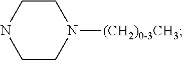

wherein
the number of contiguous atoms present in a span:

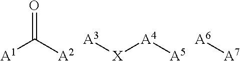

is in the range from 7-17.

As used herein, when a subscript has a value of “0”, the group is absent. For example, when A⁶ is (CH₂)₀, A⁶ is absent.

In certain embodiments of formula M5, R² is O, R³ is C═O and W is CH or N. For example, in certain embodiments of formula M5, R² is O, R³ is C═O and W is CH.

In certain embodiments of formula M5, R² is C═O, R³ is O and W is CH.

In certain embodiments of formula M5, A¹ is CH₂, A³ is (CH₂)₂₋₅, X is N, A⁴ is C═O, A⁵ is O, S, NH, NCH₃, A⁶ is (CH₂)₁₋₂, and A⁷ is (CH₂)₁₋₄. For example, in certain embodiments A¹ is CH₂, A³ is (CH₂)₂₋₅, X is N, A⁴ is C═O, A⁵ is O, A⁶ is (CH₂)₁₋₂, and A⁷ is (CH₂)₁₋₄.

In certain embodiments of formula M5, A¹ is CH₂, A³ is (CH₂)₁₋₄, X is CH, A⁴ is CH₂, NH, NCH₃, O, A⁵ is C═O, A⁶ is O, NH, NCH₃, or CH₂, and A⁷ is (CH)₂₋₆. In certain embodiments as described herein, A¹ is CH₂, A³ is (CH₂)₁₋₄, X is CH, A⁴ is NH, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is NH, A⁵ is C═O, A⁶ is O, and A⁷ is (CH)₂₋₆. For example, in certain embodiments of formula M5, A¹ is CH₂, A³ is (CH₂)₁₋₄, X is CH, A⁴ is NH, A⁵ is C═O, A⁶ is CH₂, and A⁷ is (CH)₂₋₆. In certain embodiments as described herein, A¹ is CH₂, A³ is (CH₂)₁₋₄, X is CH, A⁴ is CH₂, A⁵ is C═O, A⁶ is O, NH, NCH₃, or CH₂, or A⁷ is (CH)₂₋₆. For example, in certain embodiments of formula M5, A¹ is CH₂, A³ is (CH₂)₁₋₄, X is CH, A⁴ is CH₂, A⁵ is C═O, A⁶ is O, A⁷ is (CH)₂₋₆. In certain embodiments as described herein, A¹ is CH₂, A³ is (CH₂)₁₋₄, X is CH, A⁴ is O, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is O, A⁵ is C═O, A⁶ is CH₂, and A⁷ is (CH)₂₋₆. In certain embodiments as described herein, A¹ is CH₂, A³ is (CH₂)₁₋₄, X is CH, A⁴ is NCH₃, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is NCH₃, A⁵ is C═O, A⁶ is CH₂, and A⁷ is (CH)₂₋₆.

In certain embodiments of formula M5, A¹ is (CH₂)₂, A³ is (CH₂)₁₋₄, X is C—CH₃, A⁴ is C═O, A⁵ is O, NH, NCH₃, A⁶ is (CH₂)₁₋₂, or A⁷ is (CH₂)₁₋₄. For example, in certain embodiments, A¹ is (CH₂)₂, A³ is (CH₂)₁₋₄, X is C—CH₃, A⁴ is C═O, A⁵ is O, A⁶ is (CH₂)₁₋₂, or A⁷ is (CH₂)₁₋₄.

In certain embodiments of formula M5, the number of contiguous connective atoms present in a span:

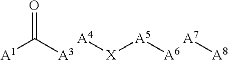

is in the range from 7-17. For example, in certain embodiments, the number of contiguous connective atoms present in a span:

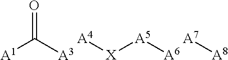

is in the range of 7-11 or 7-10. In certain embodiments, the number of contiguous connective atoms present in a span:

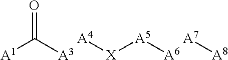

is in the range of 10-17 (e.g., in the range of 10-16, or 10-14, or 10-12). For example, in certain embodiments, the number of contiguous connective atoms present in a span:

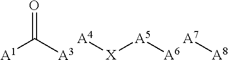

is 10. For example, in certain embodiments, the number of contiguous connective atoms present in a span:

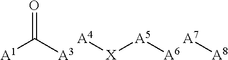

is 7. The present inventors have found that changing the number of contiguous connective atoms present in each span can allow for tuning of the pKa of the cationic lipid.

In some embodiments of formula M5, Y is

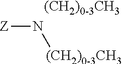

and Z is a bond. In some embodiments of formula M5, Y is

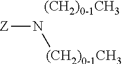

and Z is a bond. For example, in some embodiments of formula M5, Y is

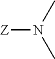

and Z is a bond.

In some embodiments of formula M5, Y is

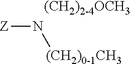

and Z is a bond. For example, on some embodiments of formula M5, Y is

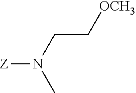

and Z is a bond.

In some embodiments of formula M5, Y is

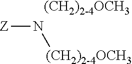

and Z is a bond. For example, in some embodiments of formula M5, Y is

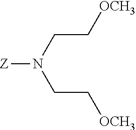

and Z is a bond.

In some embodiments of formula M5, Y is

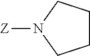

and Z is a bond.

In some embodiments of formula M5, Y is

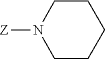

and Z is a bond.

In some embodiments of formula M5, Y is

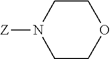

and Z is a bond.

In some embodiments of formula M5, Y is

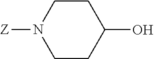

and Z is a bond.

In some embodiment of formula M5, Y is

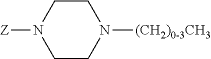

and Z is a bond.

In some embodiment of formula M5, Y is

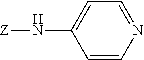

and Z is a bond.

In some embodiment of formula M5, Y is

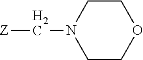

and Z is a bond.

In some embodiment of formula M5, Y is

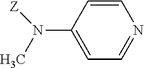

and Z is a bond.

In some embodiment of formula M5, Y is

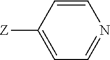

and Z is a bond.

In some embodiment of formula M5, Y is

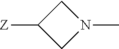

and Z is a bond.

In some embodiment of formula M5, Y is

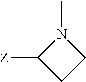

and Z is a bond.

In some embodiment of formula M5, Y is

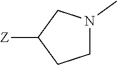

and Z is a bond.

In some embodiment of formula M5, Y is

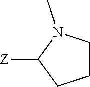

and Z is a bond.

In some embodiment of formula M5, Y is

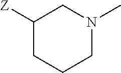

and Z is a bond.

In some embodiment of formula M5, Y is

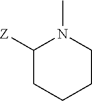

and Z is a bond.

In some embodiment of formula M5, Y is

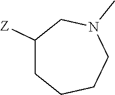

and Z is a bond.

In some embodiments of formula M5, Y is

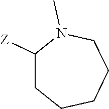

and Z is a bond.

In some embodiments of formula M5, Y is

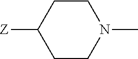

and Z is a bond.

In some embodiments of formula M5, Y is

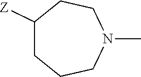

and Z is a bond.

In some embodiments, the ionizable cationic lipid has the structure CICL

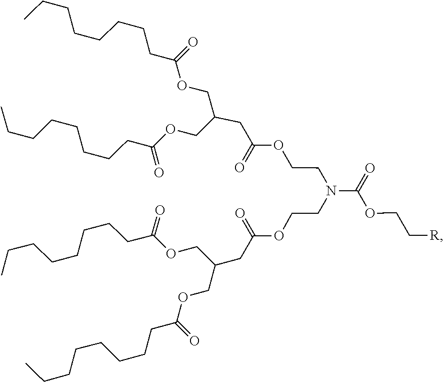

wherein R is

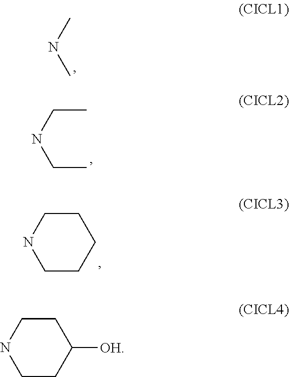

In certain embodiments, the ionizable cationic lipid of CICL is referred to as CICL1 when R is

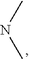

that is

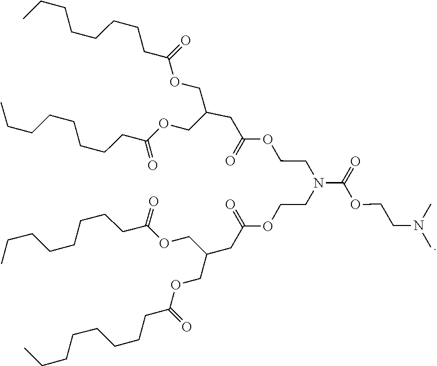 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN(C)C]

In certain embodiments, the ionizable cationic lipid of CICL is referred to as CICL2 when R is

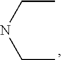

that is

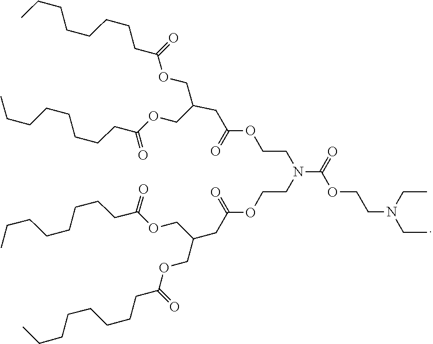 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN(CC)CC]

In certain embodiments, the ionizable cationic lipid of CICL is referred to as CICL3 when R is

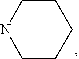

that is

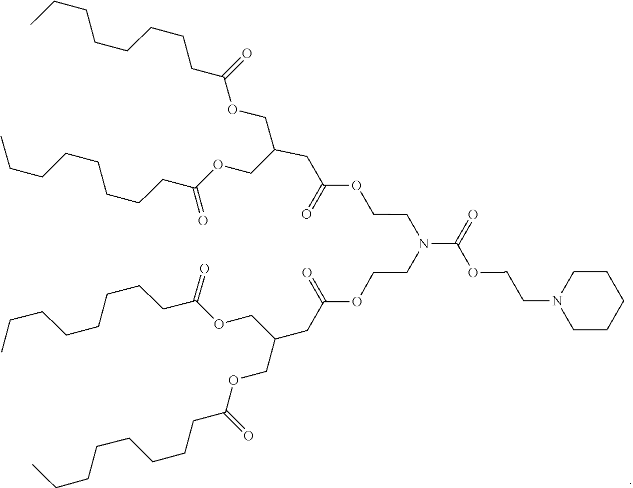 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN1CCCCC1]

In certain embodiments, the ionizable cationic lipid of CICL is referred to as CICL4 when R is

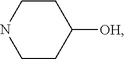

that is

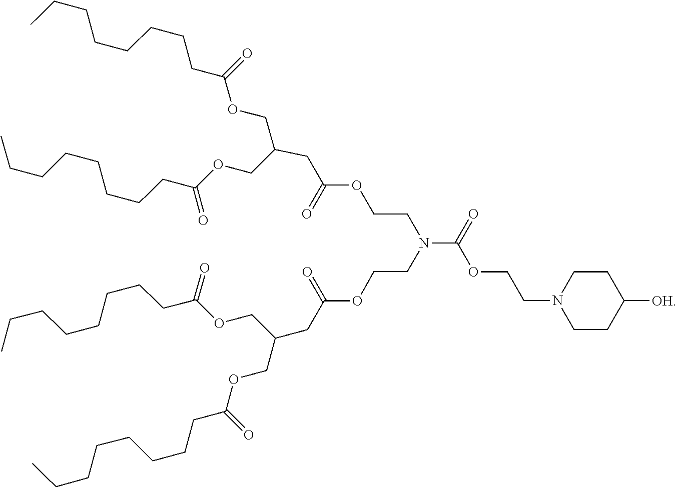 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN1CCC(O)CC1]

In some embodiments, the ionizable cationic lipid has the structure CICL-IE:

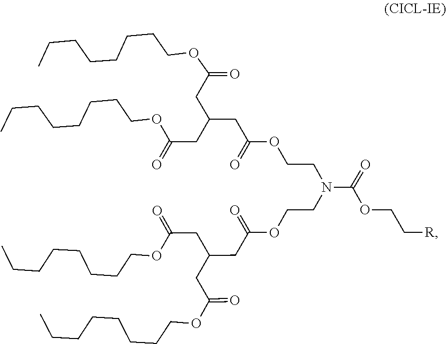

wherein R is

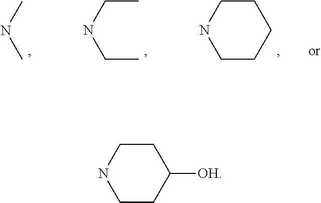

In certain embodiments, the ionizable cationic lipid of CICL-IE is referred to as CICL250 when R is

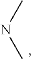

that is

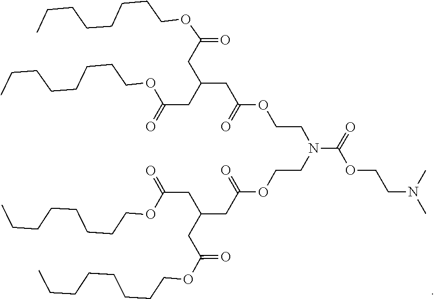 [SMILES: CCCCCCCCOC(=O)CC(CC(=O)OCCCCCCCC)CC(=O)OCCN(CCOC(=O)CC(CC(=O)OCCCCCCCC)CC(=O)OCCCCCCCC)C(=O)OCCN(C)C]

In certain embodiments, the ionizable cationic lipid of CICL-IE is referred to as CICL250.2 when R is

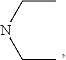

that is

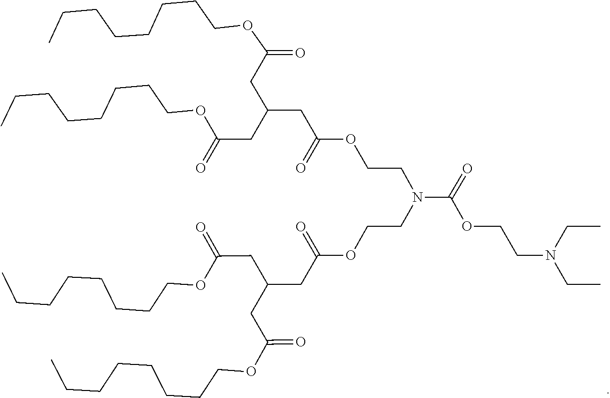 [SMILES: CCCCCCCCOC(=O)CC(CC(=O)OCCCCCCCC)CC(=O)OCCN(CCOC(=O)CC(CC(=O)OCCCCCCCC)CC(=O)OCCCCCCCC)C(=O)OCCN(CC)CC]

In certain embodiments, the ionizable cationic lipid of CICL-IE is referred to as CICL250.3 when R is

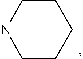

that is

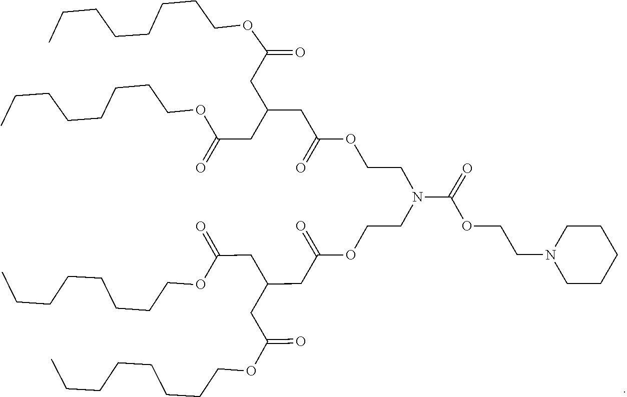 [SMILES: CCCCCCCCOC(=O)CC(CC(=O)OCCCCCCCC)CC(=O)OCCN(CCOC(=O)CC(CC(=O)OCCCCCCCC)CC(=O)OCCCCCCCC)C(=O)OCCN1CCCCC1]

In certain embodiments, the ionizable cationic lipid of CICL-IE is referred to as CICL250.4 when R is

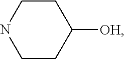

that is

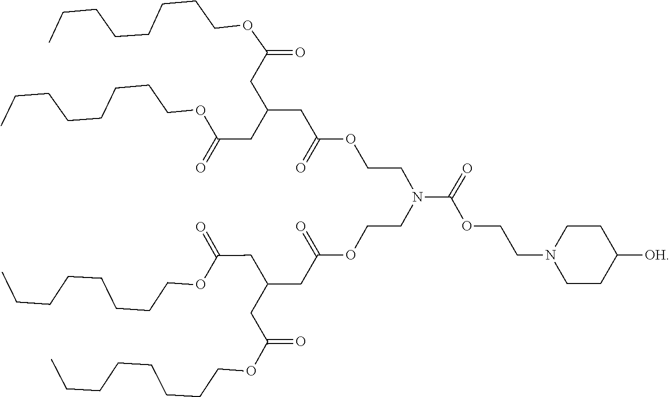 [SMILES: CCCCCCCCOC(=O)CC(CC(=O)OCCCCCCCC)CC(=O)OCCN(CCOC(=O)CC(CC(=O)OCCCCCCCC)CC(=O)OCCCCCCCC)C(=O)OCCN1CCC(O)CC1]

In certain embodiments, an ionizable cationic lipid has a structure of formula M1:

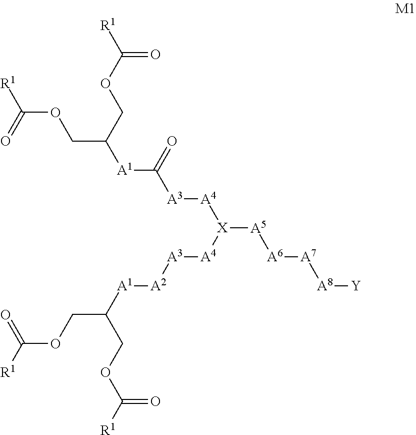

wherein
each R¹ is independently selected from a C₇-C₁₁ alkyl or a C₇-C₁₁ alkenyl,
A¹ is CH₂ or CH₂CH₂,
A³ is O,
A⁴ is CH₂ or CH₂CH₂, wherein A⁴ is not CH₂ if X is N,
X is N, CH or C—CH₃,
A⁵ is CH₂, C═O, NH, NCH₃, or O,
A⁶ is O, S, NH, NCH₃, or C═O, if A⁵ is C═O, or A⁶ is C═O if A⁵ is not C═O
A⁷ is (CH₂)₀₋₄, O, S, NH, NCH₃,
A⁸ is (CH₂)₀₋₄, wherein if A⁷ is O, S, NH, NCH₃, A⁸ is (CH₂)₂₋₄, and
Y is

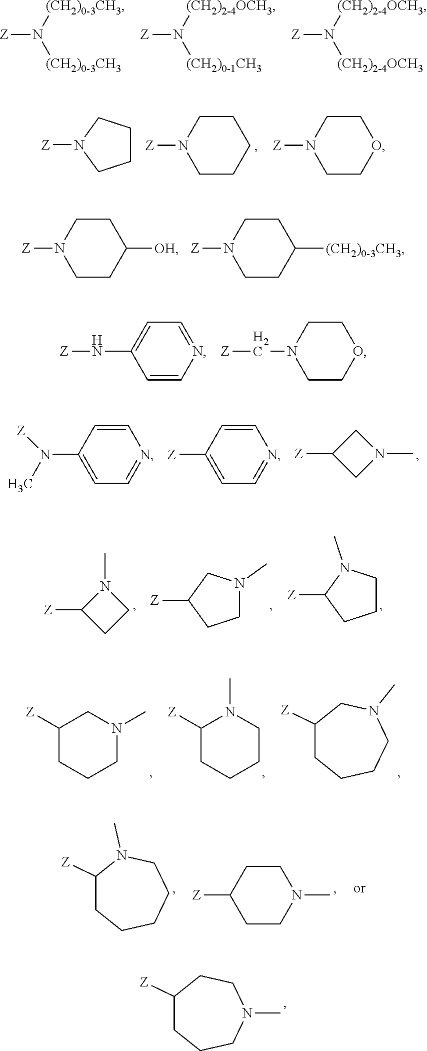

wherein Z is a bond; and
   wherein A⁷ and A⁸ are not both (CH₂)₀ unless A⁶ is C═O,
and
when A¹ and A⁴ are CH₂ then X is CH, A⁵ is CH₂, NH, NCH₃, or O, A⁶ is C═O, A⁷ is O, S, NH, NCH₃, or (CH₂)₀₋₄, and A⁸ is (CH₂)₀₋₄, or
when A¹ is CH₂ and A⁴ is CH₂ or CH₂CH₂, X is CH and A⁷ is S, then A⁵ is NH, or NCH₃, A⁶ is C═O, and A⁸ is (CH₂)₂₋₄, or
when A¹ is CH₂, A⁴ is CH₂CH₂, and X is CH, then A⁵ is NH, NCH₃, or O, A⁶ is C═O, A⁷ is (CH₂)₁₋₄, and A⁸ is (CH₂)₁₋₄, or
when A¹ is CH₂, A⁴ is CH₂CH₂, and X is N, then A⁵ is C═O, A⁶ is O or S, A⁷ is (CH₂)₀₋₄, and A⁸ is (CH₂)₀₋₄, wherein A⁶ is not bonded directly to a nitrogen, or
when A¹ is CH₂CH₂ then A⁴ is CH₂, X is C—CH₃, A⁵ is C═O, A⁶ is O, NH, or NCH₃,
A⁷ is (CH₂)₀₋₄, and A⁸ is (CH₂)₀₋₄, wherein A⁶ is not bonded directly to a nitrogen.

In certain embodiments of formula M1, A¹ and A⁴ are CH₂, X is CH, A⁵ is CH₂, NH, NCH₃, or O, A⁶ is C═O, A⁷ is O, S, NH, NCH₃, or (CH₂)₀₋₄, and A⁸ is CH₂CH₂.

In certain embodiments of formula M1, A¹ is CH₂ and A⁴ is CH₂ or CH₂CH₂, X is CH and A⁷ is S, then A⁵ is NH or NCH₃, A⁶ is C═O, and A⁸ is (CH₂)₂₋₄.

In certain embodiments of formula M1, A¹ is CH₂, A⁴ is CH₂CH₂, X is CH,
A⁵ is NH, NCH₃, or O, A⁶ is C═O, A⁷ is CH₂, and A⁸ is CH₂.

In certain embodiments of formula M1, A¹ is CH₂, A⁴ is CH₂CH₂, X is N, A⁵ is C═O, A⁶ is O or S, A⁷ is CH₂, and A⁸ is CH₂.

In certain embodiments of formula M1, A¹ is CH₂CH₂, A⁴ is CH₂, X is C—CH₃, A⁵ is C═O, A⁶ is O, NH, or NCH₃, A⁷ is CH₂, and A⁸ is CH₂.

In certain aspects, the constrained ionizable cationic lipids of this disclosure have a structure of the formula M6:

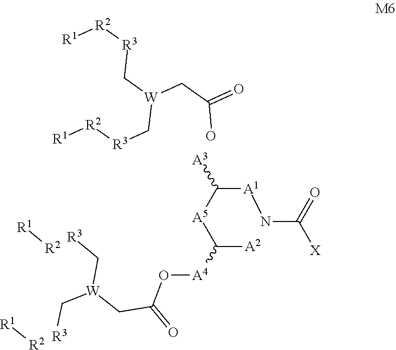

wherein X is

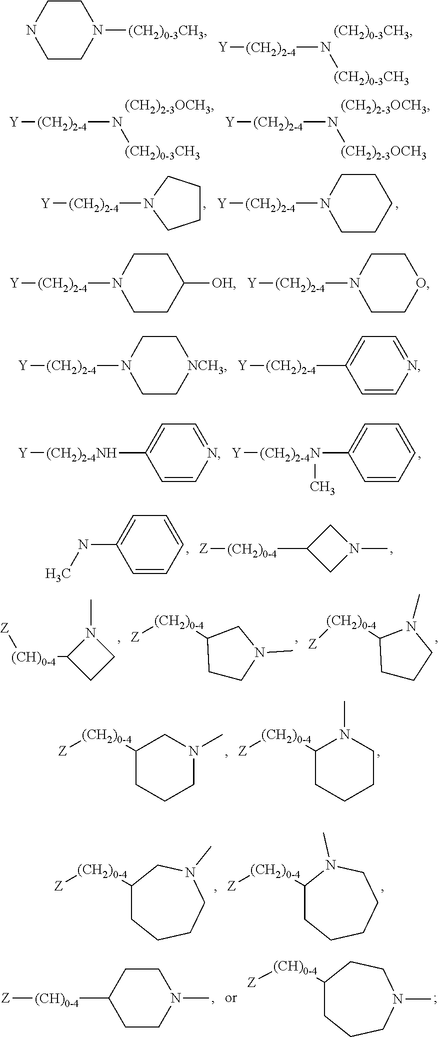

and
Y is O, S, NH, or NCH₃;
Z is O, NH, or NCH₃;
R² is O, R³ is C═O and W is CH or N, or R² is C═O, R³ is O and W is CH; and
each R¹ is independently selected from a C₇-C₁₁ alkyl or a C₇-C₁₁ alkenyl;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
a wavy bond indicates that any relative or absolute stereo-configuration of the corresponding ring atom, or a mixture of stereo-configurations, can be assumed.

As used herein, when a subscript has a value of “0”, the group is absent. For example, when A¹ is (CH₂)₀, A¹ is absent.

In certain embodiments of formula M6, R² is O, R³ is C═O and W is CH or N. For example, in certain embodiments of formula M6, R² is O, R³ is C═O and W is CH.

In certain embodiments of formula M6, R² is C═O, R³ is O and W is CH.

In various embodiments of M6, A¹ through A⁴ are chosen so that there are only two main chain atoms between the ring nitrogen and each nearest ester oxygen in the nearest tail group.

In certain embodiments of M6, A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

In certain embodiments of M6, A¹ is (CH₂)₀, A² is (CH₂)₁, A³ is (CH₂)₁, A⁴ is (CH₂)₀, and A⁵ is (CH₂)₁.

In certain embodiments of M6, A¹ is (CH₂)₁, A² is (CH₂)₁, A³ is (CH₂)₀, A⁴ is (CH₂)₀, and A⁵ is (CH₂)₀.

In certain embodiments of M6, A¹ is (CH₂)₁, A² is (CH₂)₁, A³ is (CH₂)₀, A⁴ is (CH₂)₀, and A⁵ is (CH₂)₁.

In certain embodiments of M6, A¹ is (CH₂)₁, A² is (CH₂)₁, A³ is (CH₂)₀, A⁴ is (CH₂)₀, and A⁵ is (CH₂)₂ or CH═CH.

In some embodiments of formula M6 as described herein, X is

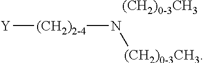

For example, in some embodiments of formula M6, X is

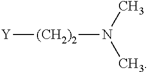

In some embodiments of formula M6 as described herein, X is

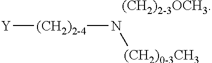

In some embodiments of formula M6 as described herein, X is

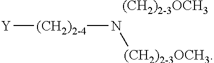

In some embodiments of formula M6 as described herein, X is

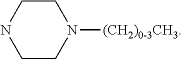

In some embodiments X is

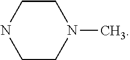

In some embodiments of formula M6 as described herein, X is

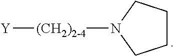

In some embodiments of formula M6 as described herein, X is

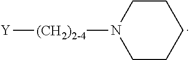

In some embodiments of formula M6 as described herein, X is

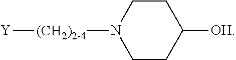

In some embodiments of formula M6 as described herein, X is

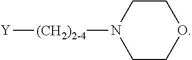

In some embodiments of formula M6 as described herein, X is

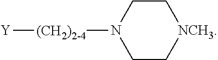

In some embodiments of formula M6 as described herein, X is

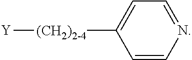

In some embodiments of formula M6 as described herein, X is

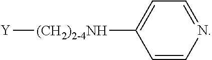

In some embodiments of formula M6 as described herein, X is

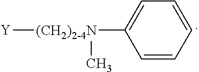

In some embodiments of formula M6 as described herein, X is

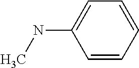

In some embodiments of formula M6, X is

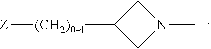

In some embodiments of formula M6, X is

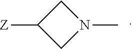

In some embodiments of formula M6, X is

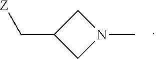

In some embodiments of formula M6, X is

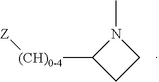

In some embodiments of formula M6, X is

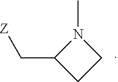

In some embodiments of formula M6, X is

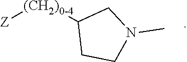

In some embodiments of formula M6, X is

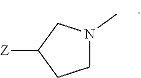

In some embodiments of formula M6, X is

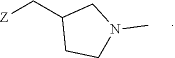

In some embodiments of formula M6, X is

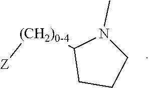

In some embodiments of formula M6, X is

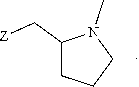

In some embodiments of formula M6, X is

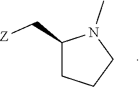

In some embodiments of formula M6, X is

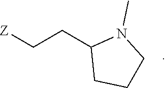

In some embodiments of formula M6, X is

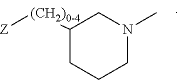

In some embodiments of formula M6, X is

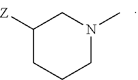

In some embodiments of formula M6, X is

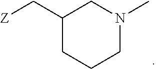

In some embodiments of formula M6, X is

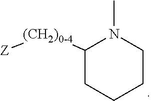

In some embodiments of formula M6, X is

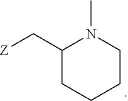

In some embodiments of formula M6, X is

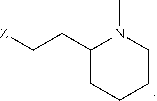

In some embodiments of formula M6, X is

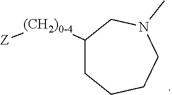

In some embodiments of formula M6, X is

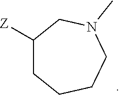

In some embodiments of formula M6, X is

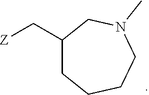

In some embodiments of formula M6, X is

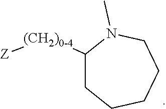

In some embodiments of formula M6, X is

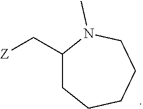

In some embodiments of formula M6, X is

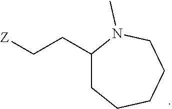

In some embodiments of formula M6, X is

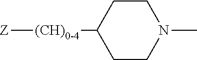

In some embodiments of formula M6, X is

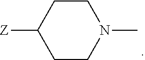

In some embodiments of formula M6, X is

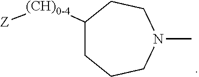

In some embodiments of formula M6, X is

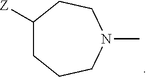

As described above, in some embodiments of formula M6, Y can be selected from O, S, NH, or NCH₃. In some embodiments of formula M6, Y is O. In some other embodiments of formula M6, Y is S.

In some embodiments of formula M6, X is

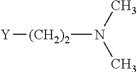

and Y is O. In some embodiments of formula M6, X is

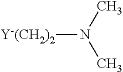

### and Y is S.

As described above, Z can be selected from O, NH, or NCH₃. In some embodiments, Z is O.

In some embodiments of formula M6, X is

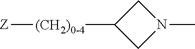

and Z is O. In some embodiments of formula M6, X is

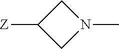

and Z is O. In some embodiments of formula M6, X is

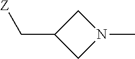

### and Z is O.

In some embodiments of formula M6, X is

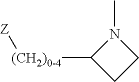

and Z is O. In some embodiments of formula M6, X is

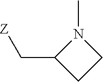

### and Z is O.

In some embodiments of formula M6, X is

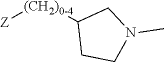

and Z is O. In some embodiments of formula M6, X is

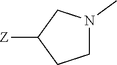

and Z is O. In some embodiments of formula M6, X is

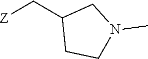

### and Z is O.

In some embodiments of formula M6, X is

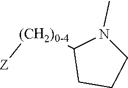

and Z is O. In some embodiments of formula M6, X is

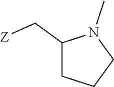

and Z is O. In some embodiments of formula M6, X is

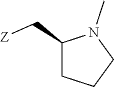

and Z is O. In some embodiments of formula M6, X is

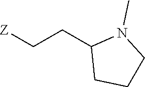

### and Z is O.

In some embodiments of formula M6, X is

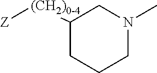

and Z is O. In some embodiments of formula M6, X is

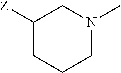

and Z is O. In some embodiments of formula M6, X is

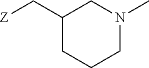

### and Z is O.

In some embodiments of formula M6, X is

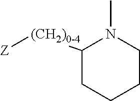

and Z is O. In some embodiments of formula M6, X is

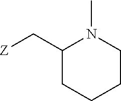

and Z is O. In some embodiments of formula M6, X is

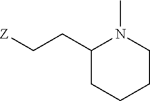

### and Z is O.

In some embodiments of formula M6, X is

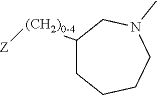

and Z is O. In some embodiments of formula M6, X is

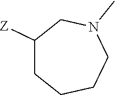

and Z is O. In some embodiments of formula M6, X is

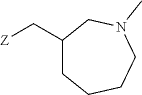

### and Z is O.

In some embodiments of formula M6, X is

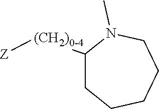

and Z is O. In some embodiments of formula M6, X is

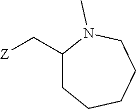

and Y is O. In some embodiments of formula M6, X is

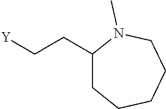

### and Z is O.

In some embodiments of formula M6, X is

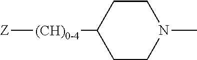

and Z is O. In some embodiments of formula M6, X is

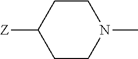

### and Z is O.

In some embodiments of formula M6, X is

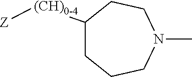

and Z is O. In some embodiments of formula M6, X is

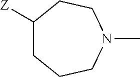

### and Z is O.

As described above, for both formula M5 and M6, each R¹ is independently selected from C₇-C₁₁ alkyl or C₇-C₁₁ alkenyl. In some embodiments of formula M5 and/or M6, each R¹ is independently selected from C₇-C₁₁ alkyl, e.g., C₇-C₁₀ alkyl, or C₇-C₉ alkyl. In certain embodiments of formula M5 and/or M6, each R¹ is independently selected from a linear C₇-C₁₁ alkyl, e.g., a linear C₇-C₁₀ alkyl, or a linear C₇-C₉ alkyl. In some embodiments of formula M5 and/or M6 as described herein, each R¹ is independently selected from (CH₂)₆₋₈CH₃. In some of these and other embodiments, R¹ is (CH₂)₇CH₃. In some embodiments of formula M5 and/or M6, each R¹ is independently selected from a linear C₇-C₁₁ alkenyl, e.g., a linear C₇-C₁₀ alkenyl, or a linear C₇-C₉ alkenyl. For example, in some embodiments of formula M5 and/or M6, each R¹ is a linear C₈ alkenyl. In certain other embodiments of formula M5 and/or M6, each R¹ is independently selected from a branched C₇-C₁₁ alkyl, e.g., C₇-C₁₀ alkyl, or C₇-C₉ alkyl. For example, in some embodiments of formula M5 and/or M6, each R¹ is a branched C₈ alkyl. In certain embodiments of formula M5 and/or M6, each R¹ is independently selected from a branched C₇-C₁₁ alkenyl, e.g., C₇-C₁₀ alkenyl, or C₇-C₉ alkenyl. For example, in some embodiments of formula M5 and/or M6, each R¹ is a branched C₈ alkenyl. In some embodiments of formula M5 and/or M6, wherein R¹ is a branched alkyl or alkenyl, the branch point is positioned so that ester carbonyls are not in an α position relative to the branch point, for example they are in a β position relative to the branch point.

In certain embodiments of formula M5 and/or M6 as described herein, each R¹ is the same. In certain embodiments of formula M5 and/or M6, each R¹ nearest a common branch point is the same, but those nearest a first common branch point differ from those nearest a second common branch point. In certain embodiments of formula M5 and/or M6, each R¹ nearest a common branch point is different but the pair of R¹s nearest a first common branch point is the same the pair nearest a second common branch point.

In certain embodiments of formula M6, the ionizable cationic lipid is substantially enantiomerically pure (e.g., at least about 80%, at least about 85%, at least about 90%, at least about 95%, at least about 96%, at least about 97%, at least about 98%, or at least about 99%). In certain embodiments of formula M6, the ionizable cationic lipid is a racemic mixture. In certain embodiments of formula M6, the ionizable cationic lipid is a mixture of two or more stereoisomers. In certain embodiments of formula M6, at least two of the two or more stereoisomers are diastereomers. In certain embodiments of formula M6, at least two of the two or more stereoisomers are enantiomers.

In certain embodiments an ionizable cationic lipid has a structure of formula M2:

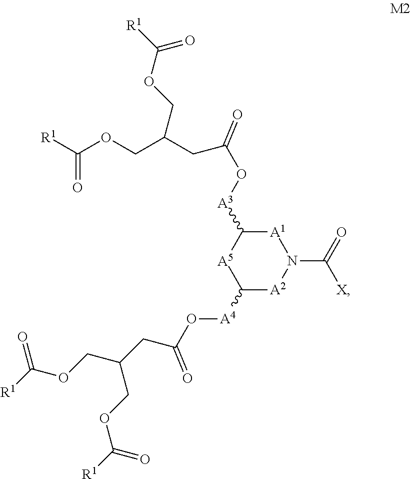

wherein X is

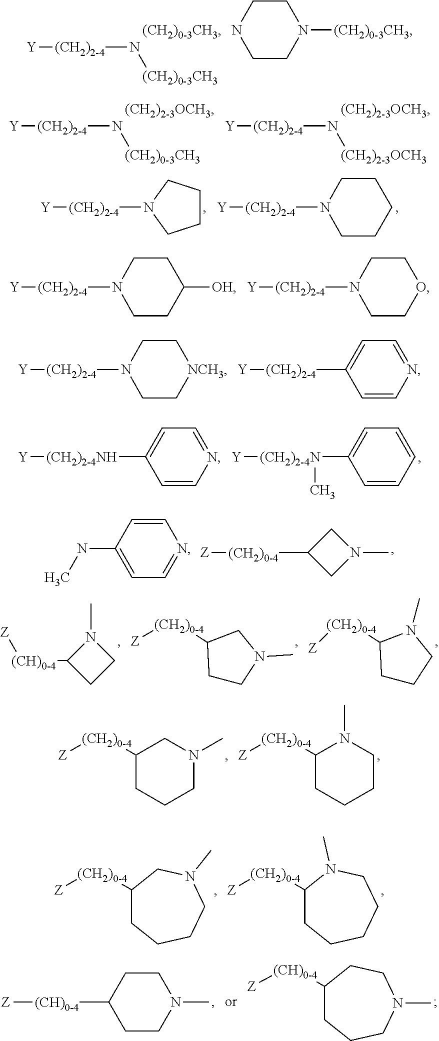

and
Y is O, S, NH, or NCH₃;
Z is O, NH, or NCH₃,
each R¹ is independently selected from C₇-C₁₁ alkyl or C₇-C₁₁ alkenyl;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
the wavy bonds indicate that any relative or absolute stereo-configuration of the corresponding ring atom, or a mixture stereo-configurations, can be assumed.

In various embodiments, A¹ through A⁴ are chosen so that there are only 2 main chain atoms between the ring nitrogen and each nearest ester oxygen in the nearest tail group.

In certain embodiments, A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

In certain embodiments, A¹ is (CH₂)₀, A² is (CH₂)₀, A³ is (CH₂)₁, A⁴ is (CH₂)₁, and A⁵ is (CH₂)₁;

In certain embodiments, A¹ is (CH₂)₀, A² is (CH₂)₁, A³ is (CH₂)₁, A⁴ is (CH₂)₀, and A⁵ is (CH₂)₁;

In certain embodiments, A¹ is (CH₂)₁, A² is (CH₂)₁, A³ is (CH₂)₀, A⁴ is (CH₂)₀, and A⁵ is (CH₂)₀;

In certain embodiments, A¹ is (CH₂)₁, A² is (CH₂)₁, A³ is (CH₂)₀, A⁴ is (CH₂)₀, and A⁵ is (CH₂)₁.

In certain embodiments, A¹ is (CH₂)₁, A² is (CH₂)₁, A³ is (CH₂)₀, A⁴ is (CH₂)₀, and A⁵ is (CH₂)₂ or CH═CH.

The synthesis of lipids having the structure of Formulas 1, 2, 3, M1, M2, M5, CICL, CICL-IE, or M6 is described in PCT/US2024/049649 (M2, some M6 embodiments), PCT/US2024/049627 (M1, some M5), U.S. Patent Application Nos. 63/632,940 and 63,654,704 (CICL-IE, some M5), and 63/632,944 and 63/654,720 (some M6), and US Patent Application Publication No. 2023/0320995 (1, 2, 3, CICL) each of which is incorporated by reference in its entirety for all that it teaches about the synthesis of such lipids, as well as particular subgenera and individual species of lipids.

In some embodiments, an LNP or tLNP comprises about 35 mol % to about 65 mol %, about 40 mol % to about 62 mol %, or about 54 mol % to about 60 mol % ionizable cationic lipid. In some embodiments, the lipid composition is at least 40 mol % and/or does not exceed 62 mol % ionizable cationic lipid. In certain embodiments, an LNP of tLNP comprises about 54 mol %, about 58 mol %, or about 62 mol % ionizable cationic lipid. In further embodiments an LNP comprises 35 mol % to 65 mol %, 40 mol % to 62 mol %, or 54 mol % to 60 mol % ionizable cationic lipid. In still further embodiments, an LNP has at least 40 mol % or does not exceed 62 mol % ionizable cationic lipid. In certain embodiments, an LNP comprises 54 mol %, 58 mol %, or 62 mol % ionizable cationic lipid.

With respect to the LNP or the tLNP, in some embodiments the ratio of total lipid to nucleic acid is 10:1 to 50:1 on a weight basis. In some embodiments, that ratio of total lipid to nucleic acid is 10:1, 20:1, 30:1, or 40:1 to 50:1, or 10:1 to 20:1, 30:1, 40:1 or 50:1, or any range bound by a pair of these ratios.

Particular compositions for LNP precursors to tLNPs and tLNPs are disclosed in U.S. Provisional Patent application No. 63/505,424 filed May 31, 2023, 63/510,061 filed Jun. 23, 2023, and 63/520,303 filed Aug. 17, 2023, as well as PCT Application No. PCT/US2023/72426 (WO2024040195A1) filed Aug. 17, 2023, each of which is incorporated by reference in its entirety. LNP and tLNP compositions can include those of Table 15. In various embodiments, N/P can be from 3 to 9 or any integer-bound sub-range in that range or about any integer in that range.

TABLE 15
LNP Compositions

| Composition | | |
|---|---|---|
| Code | Lipid Composition [Ratios] | N/P |
| BF1 | ALC-0315:DSPC:CHOL:DMG-PEG(2k):DSPE-PEG(2k)-MAL | 6 |
| | [50:10:38.5:1.4:0.1] | |
| F1 | CICL1:DSPC:CHOL:DMG-PEG(2k):DSPE-PEG(2k)-MAL | 6 |
| | [50:10:38.5:1.4:0.1] | |
| F2 | CICL1:DSPC:CHOL:DMG-PEG(2k):DSPE-PEG(2k)-MAL | 3 |
| | [50:10:38.5:1.3:0.2] | |
| F3 | CICL1:DSPC:CHOL:DMG-PEG(2k):DSPE-PEG(2k)-MAL | 9 |
| | [50:10:38.5:1.425:0.075] | |
| F4 | CICL1:DSPC:CHOL:DMG-PEG(2k):DSPE-PEG(2k)-MAL | 6 |
| | [42:10:46.5:1.4:0.1] | |
| F5 | CICL1:DSPC:CHOL:DMG-PEG(2k):DSPE-PEG(2k)-MAL | 6 |
| | [58:10:30.5:1.4:0.1] | |
| F6 | CICL1:DSPC:CHOL:DSG-PEG(2k):DSPE-PEG(2k)-MAL | 6 |
| | [35:10:53.5:1.4:0.1] | |
| F7 | CICL1:DSPC:CHOL:DSG-PEG(2k):DSPE-PEG(2k)-MAL | 6 |
| | [42:10:46.5:1.4:0.1] | |
| F8 | CICL1:DSPC:CHOL:DSG-PEG(2k):DSPE-PEG(2k)-MAL | 6 |
| | [50:10:38.5:1.4:0.1] | |
| F9 | CICL1:DSPC:CHOL:DSG-PEG(2k):DSPE-PEG(2k)-MAL | 6 |
| | [58:10:30.5:1.4:0.1] | |
| F10 | CICL1:DSPC:CHOL:DSG-PEG(2k):DSPE-PEG(2k)-MAL | 6 |
| | [62:10:26.5:1.4:0.1] | |
| F11 | CICL1:DSPC:CHOL:DMG-PEG(2k):DSPE-PEG(2k)-MAL | 6 |
| | [58:7:33.5:1.4:0.1] | |
| F12 | CICL1:DSPC:CHOL:DPG-PEG(2k):DSPE-PEG(2k)-MAL | 6 |
| | [58:7:33.5:1.4:0.1] | |
| F13 | CICL1:DSPC:CHOL:DSG-PEG(2k):DSPE-PEG(2k)-MAL | 6 |
| | [58:7:33.5:1.4:0.1] | |
| F14 | CICL1:DSPC:CHOL:DMG-PEG(2k):DSPE-PEG(2k)-MAL | 6 |
| | [58:7:34:0.9:0.1] | |
| F15 | CICL1:DSPC:CHOL:DSG-PEG(2k):DSPE-PEG(2k)-MAL | 6 |
| | [58:10:30:1.9:0.1] | |
| F16 | CICL1:DSPC:CHOL:DSG-PEG(2k):DSPE-PEG(2k)-MAL | 6 |
| | [50:10:39.5:0.4:0.1] | |
| F17 | CICL1:DSPC:CHOL:DSG-PEG(2k):DSPE-PEG(2k)-MAL | 6 |
| | [50:10:39:0.9:0.1] | |
| F18 | CICL1:DSPC:CHOL:DSG-PEG(2k):DSPE-PEG(2k)-MAL | 6 |
| | [50:10:38.5:1.4:0.1] | |
| F19 | CICL1:DSPC:CHOL:DSG-PEG(2k):DSPE-PEG(2k)-MAL | 6 |
| | [50:10:38:1.9:0.1] | |
| F20 | CICL1:DSPC:CHOL:DSG-PEG(2k):DSPE-PEG(2k)-MAL | 6 |
| | [50:10:37.5:2.4:0.1] | |
| F21 | CICL1:DSPC:CHOL:DSG-PEG(2k):DSPE-PEG(2k)-MAL | 6 |
| | [50:10:37:2.9:0.1] | |
| F22 | CICL1:DSPC:CHOL:DSG-PEG(2k):DSPE-PEG(2k)-MAL | 6 |
| | [58:10:31:0.9:0.1] | |
| F23 | CICL1:DSPC:CHOL:DSG-PEG(2k):DSPE-PEG(2k)-MAL | 6 |
| | [58:10:30:1.9:0.1] | |
| F24 | CICL1:DSPC:CHOL:DSG-PEG(2k):DSPE-PEG(2k)-MAL | 6 |
| | [58:10:29.5:2.4:0.1] | |
| F25 | CICL1:DSPC:CHOL:DSPE-PEG(0.75k):DSPE-PEG(2k)-MAL | 6 |
| | [58:10:30.5:1.4:0.1] | |
| F26 | CICL1:DSPC:CHOL:DSPE-PEG(1k):DSPE-PEG(2k)-MAL | 6 |
| | [58:10:30.5:1.4:0.1] | |
| F27 | CICL1:DSPC:CHOL:DMPE-PEG(1k):DSPE-PEG(2k)-MAL | 6 |
| | [58:10:30.5:1.4:0.1] | |
| F29 | CICL1:DSPC:CHOL:DSG-PEG(5k):DSPE-PEG(5k)-MAL | 6 |
| | [58:10:31.4:0.5:0.1] | |
| F30 | CICL1:DSPC:CHOL:DMG-PEG(2k):DSG-PEG(2k)-MAL | 6 |
| | [58:10:30.5:1.4:0.1] | |
| F31 | CICL1:DSPC:CHOL:DSG-PEG(2k):DSG-PEG(2k)-MAL | 6 |
| | [58:10:30.5:1.4:0.1] | |
| F32 | CICL1:DSPC:CHOL:DSPE-PEG(2k):DSPE-PEG(2k)-MAL | 6 |
| | [58:10:30.5:1.4:0.1] | |
| F33 | CICL1:DSPC:CHOL:DSG-PEG(2k):DSPE-PEG(5k)-MAL | 6 |
| | [58:10:30.5:1.4:0.1] | |
| F34 | CICL1:DSPC:CHOL:DSG-PEG(2k):DSPE-PEG(2k)-MAL | 6 |
| | [58:13:27.5:1.4:0.1] | |
| F35 | CICL1:DMPC:CHOL:DSG-PEG(2k):DSPE-PEG(2k)-MAL | 6 |
| | [58:10:30.5:1.4:0.1] | |
| F36 | CICL1:DPPC:CHOL:DSG-PEG(2k):DSPE-PEG(2k)-MAL | 6 |
| | [58:10:30.5:1.4:0.1] | |
| F37 | CICL1:DAPC:CHOL:DSG-PEG(2k):DSPE-PEG(2k)-MAL | 6 |
| | [58:10:30.5:1.4:0.1] | |
| F38 | CICL1:18:1 PA:CHOL:DSG-PEG(2k):DSPE-PEG(2k)-MAL | 6 |
| | [58:10:30.5:1.4:0.1] | |
| F40 | CICL1:DSPC:CHOL:20(S)-Hydroxycholesterol:DSG- | 6 |
| | PEG(2k):DSPE-PEG(2k)-MAL [58:10:22.9:7.6:1.4:0.1] | |
| F41 | CICL1:DSPC:CHOL:B-Sitosterol:DSG-PEG(2k):DSPE- | 6 |
| | PEG(2k)-MAL [58:10:22.9:7.6:1.4:0.1] | |
| F42 | CICL1:DSPC:CHOL:B-Sitosterol:DSG-PEG(2k):DSPE- | 6 |
| | PEG(2k)-MAL [58:10:15.25:15.25:1.4:0.1] | |

In certain embodiments, the LNP composition is composition F5 CICL1:DSPC:CHOL:DMG-PEG2000:DSPE-PEG2000-MAL [58:10:30.5:1.4:0.1] or composition F9 CICL1:DSPC:CHOL:DSG-PEG2000:DSPE-PEG2000-MAL [58:10:30.5:1.4:0.1]. The terminal group of the non-functionalized PEG can be methoxy. The N/P ratio (the ratio of positively-chargeable lipid amine (N=nitrogen) groups to negatively-charged nucleic acid phosphate (P) groups) can be 6. After initial LNP formation a SATA-modified anti-CD5 or anti-CD8 antibody can be reacted with the maleimide moiety to provide the final tLNP. In some embodiments, CD8-targeted tLNP in which the targeting moiety comprises a humanized anti-CD8 antibody antigen binding domain have the lipid composition F9 found in Table 1S.

In some embodiments the antibody binding moiety is conjugated to the PEG moiety of the PEG-lipid through a thiol-modified lysine residue. In some embodiments, conjugation is through a cysteine residue in a native or added antibody sequence. In other embodiments, the conjugation is through a sortase A substrate sequence. In still other embodiments, conjugation is through a specific lysine residue (Lys248 or Lys288) in the Fc region, aided by a modified affinity reagent, for example, an AJICAP reagent. In certain embodiments of such embodiments, the humanized anti-CD8 antibody is linked to LNP by N-succinimidyl S-acetylthioacetate (SATA)-maleimide conjugation chemistry to form targeted LNPs (tLNPs). The antibody is beneficially first modified with SATA to introduce sulfhydryl groups at accessible lysine residues allowing conjugation to maleimide. (Some lysine residues can be buried in the interior of the protein and thus inaccessible to the SATA reagent.) Diabodies and F(ab′)₂ can be conjugated by first partially reducing cystine bonds in the antibody with tris(2-carboxy)phosphine (TCEP) to generate thiol groups for conjugation through the maleimide moieties of the LNP.

In any of the aforementioned tLNP embodiments, certain embodiments include tLNPs encapsulating one or more herein disclosed mRNA(s) encoding an anti-CD19 such as SEQ ID NOS: 130, 151, 165, or 166, or an anti-CD20 CAR such as SEQ ID NOS: 178, 319, 321, 322, 323, 360 as its payload. In any of the aforementioned tLNP embodiments, certain embodiments include tLNPs in which the targeting moiety comprises one of the herein disclosed humanized CT8 antigen binding domains, such as one that comprises a VL region having the amino acid sequence of SEQ ID NO: 196 and a VH region having the amino acid sequence of one of SEQ ID NOs: 190 or 202-204. In some such embodiments, the targeting moiety is a whole humanized anti-CD8 antibody comprising heavy chain with a silenced Fc region such as one having the amino acid sequence of SEQ ID NO: 218 or 219. In certain instances, the whole humanized anti-CD8 antibody comprising a heavy chain with a silenced Fc region comprises the sequence of CBD1033HC (SEQ ID NO: 347) and/or a light chain comprising the sequence of CBD1033LC (SEQ ID NO: 348). In some embodiments, the targeting moiety is an anti-CD8 F(ab′) of a classic F(ab′). In some embodiments, the targeting moiety is an anti-CD8 F(ab′) of an engineered F(ab′). Examples of such anti-CD8 F(ab′) of a classic F(ab′) or an engineered F(ab′) are listed in Table 18. In some embodiments, the anti-CD8 F(ab′) comprises a light chain with a wild type Kappa constant region, wherein the Kappa constant region has the amino acid sequence SEQ ID NO: 216. In some embodiments, the anti-CD8 F(ab′) comprises a light chain with an engineered Kappa constant region, wherein the Kappa constant region has the amino acid sequence SEQ ID NO: 376, or SEQ ID NO: 387. In some embodiments, the anti-CD8 F(ab′) comprises a heavy chain with a wild type IgG1 F(ab′), wherein the IgG1 F(ab′) has the amino acid sequence SEQ ID NO: 363. In some embodiments, the anti-CD8 F(ab′) comprises a heavy chain with an engineered IgG1 F(ab′), wherein the IgG1 F(ab′) has the amino acid sequence SEQ ID NO: 368, SEQ ID NO: 372, SEQ ID NO: 377, SEQ ID NO: 382, SEQ ID NO: 384, or SEQ ID NO: 386. In some embodiments, the anti-CD8 F(ab′) comprises a heavy chain with a wild type IgG4 F(ab′), wherein the IgG4 F(ab′) has the amino acid sequence SEQ ID NO: 366. In some embodiments, the anti-CD8 F(ab′) comprises a heavy chain with an engineered IgG4 F(ab′), wherein the IgG4 F(ab′) has the amino acid sequence SEQ ID NO: 370, SEQ ID NO: 374, SEQ ID NO: 380, or SEQ ID NO: 390. In some embodiments, the anti-CD8 F(ab′) comprises a light chain having the amino acid sequence of SEQ ID NO: 364, SEQ ID NO: 376, SEQ ID NO: 378, SEQ ID NO: 387, SEQ ID NO: 388, SEQ ID NO: 394, or SEQ ID NO: 399. In some embodiments, the anti-CD8 F(ab′) comprises a heavy chain having the amino acid sequence of SEQ ID NO: 365, SEQ ID NO: 369, SEQ ID NO: 373, SEQ ID NO: 379, SEQ ID NO: 383, SEQ ID NO: 385, SEQ ID NO: 389, SEQ ID NO: 392, SEQ ID NO: 393, SEQ ID NO: 395, SEQ ID NO: 396, SEQ ID NO: 397, SEQ ID NO: 398, SEQ ID NO: 400, or SEQ ID NO: 401. In some embodiments, the anti-CD8 F(ab′) comprises a heavy chain having the amino acid sequence of SEQ ID NO: 367, SEQ ID NO: 371, SEQ ID NO: 375, SEQ ID NO: 381, or SEQ ID NO: 391. In any of the aforementioned tLNP embodiments encapsulating a herein disclosed mRNA, in certain embodiments the tLNP comprises as its targeting moiety an antibody or antigen binding portion thereof comprising one of the aforementioned herein disclosed humanized CT8 antigen binding domains.

In some embodiments, the targeting moiety of a tLNP is a classic or engineered F(ab′). In some embodiments, the targeting moiety of a tLNP comprises an engineered F(ab′). Examples of such F(ab′) comprising a wild type or engineered constant region are listed in Table 18. In some such embodiments, the F(ab′) comprises a light chain with a wild type Kappa constant region, wherein the Kappa constant region has the amino acid sequence SEQ ID NO: 216. In some such embodiments, the F(ab′) comprises a light chain with an engineered Kappa constant region, wherein the Kappa constant region has the amino acid sequence SEQ ID NO: 376, or SEQ ID NO: 387. In some such embodiments, the F(ab′) comprises a heavy chain with a wild type IgG1 F(ab′) constant region, wherein the IgG1 F(ab′) constant region has the amino acid sequence SEQ ID NO: 363. In some such embodiments, the F(ab′) comprises a heavy chain with an engineered IgG1 F(ab′) constant region, wherein the IgG1 F(ab′) constant region has the amino acid sequence SEQ ID NO: 368, SEQ ID NO: 372, SEQ ID NO: 377, SEQ ID NO: 382, SEQ ID NO: 384, or SEQ ID NO: 386. In some such embodiments, the F(ab′) comprises a heavy chain with a wild type IgG4 F(ab′) constant region, wherein the IgG4 F(ab′) constant region has the amino acid sequence SEQ ID NO: 366. In some such embodiments, the F(ab′) comprises a heavy chain with an engineered IgG4 F(ab′) constant region, wherein the IgG4 F(ab′) constant region has the amino acid sequence SEQ ID NO: 370, SEQ ID NO: 374, SEQ ID NO: 380, or SEQ ID NO: 390.

In some embodiments, a tLNP comprises a F(ab′) or anti-CD8 F(ab′) comprising a S162C kappa chain substitution and an IgG1 or IgG4 CH1 F174C substitution. In some embodiments, the F(ab′) or anti-CD8 F(ab′) heavy chain further comprises an IgG1 CH1 C233S or IgG4 CH1 C127S substitution. In some embodiments, the F(ab′) or anti-CD8 F(ab′) light chain further comprises a C214S kappa chain substitution.

In some embodiments of a tLNP comprising a F(ab′) targeting moiety comprising a herein disclosed constant region, the tLNP comprises one or more ionizable cationic lipids disclosed herein. In some embodiments of a tLNP comprising a F(ab′) targeting moiety comprising a herein disclosed constant region, the tLNP comprises a LNP composition as disclosed in Table 15, for example F9.

In any of the aforementioned tLNP embodiments, in certain embodiments the mRNA has one of the combinations of UTRs disclosed herein. In some such embodiments, the 5′ UTR is mHBB in combination with PNLIP-RPS3A, RPS3A-PNLIP, PNLIP, RPS3A, or hHBA1-3× miR122 bs as the 3′ UTR. In some embodiments, the mRNA has a MaxiCAI optimized sequence. In some instances, the mRNA is RM_61355, RM_61357, RM_61461, RM_61486, RM_61488, RM_61489, RM_61581, RM_61582, RM_61639, RM_61658, RM_61659, or RM_61660. In particular embodiments, a tLNP will have the lipid content of composition F9. Any of these embodiments can further comprise an anti-CD8 targeting moiety such as a whole anti-CD8 antibody or an antigen binding portion thereof, for example, an scFv or F(ab′). In some instances, the anti-CD8 targeting moiety comprises SEQ ID NO: 196 and one of SEQ ID NOs: 190 or 202-204. In some instances, the whole anti-CD8 antibody comprises a heavy chain and a light chain having the amino acid sequence of SEQ ID NO: 347 and SEQ ID NO: 348, SEQ ID NO: 356 and SEQ ID NO: 348, SEQ ID NO: 357 and SEQ ID NO: 348, SEQ ID NO: 362 and SEQ ID NO: 348, SEQ ID NO: 357 and SEQ ID NO: 358, SEQ ID NO: 359 and SEQ ID NO: 348, SEQ ID NO: 360 and SEQ ID NO: 348, or SEQ ID NO: 361 and SEQ ID NO: 348, respectively. In some instances, the tLNP is tLNP-98219 or tLNP-982520.

### (V)(c)(7) Tolerability

Conventional LNPs deliver primarily to the liver. Liver toxicity has been the major dose limiting parameter observed with LNP-containing pharmaceuticals. For example, ONPATTRO®, comprising the ionizable lipid MC3, has a NOAEL (no observed adverse effect level) of only 0.3 mg/kg for multiple dosing in rats. A benchmark LNP comprising the ionizable cationic lipid ALC-0315, used in the SARS-CoV-2 vaccine COMIRNATY®, caused elevated levels of liver enzymes and acute phase proteins at single doses of ≥1 mg/kg in the rat. Merely attaching an antibody to the benchmark LNP partially reverses that elevation and the reversal is greater if the antibody directs the LNP to some other tissue (that is, a tLNP). However, use of a highly biodegradable ionizable cationic lipid, CICL-1 reduced delivery to the liver and associated liver enzyme and acute phase protein levels to a greater extent for LNP, antibody-conjugated LNP, and tLNP. The catabolism of lipids having a structure of Formula M5 or M6 is expected to be similar.

(V)(c)(8) Methods of Making LNPs or tLNPs

In some aspects, the present disclosure provides a method of making a LNP or tLNP comprising mixing of an aqueous solution of a nucleic acid (or other negatively charged payload) and an alcoholic solution of the lipids in proportions disclosed herein. In particular embodiments, the mixing is rapid.

The aqueous solution is buffered at pH of about 3 to about 5, for example, without limitation, with citrate or acetate. In various embodiments, the alcohol can be ethanol, isopropanol, t-butanol, or a combination thereof. In some embodiments, the rapid mixing is accomplished by pumping the two solutions through a T-junction or with an impinging jet mixer. Microfluidic mixing through a staggered herringbone mixer (SHM) or a hydrodynamic mixer (microfluidic hydrodynamic focusing), microfluidic bifurcating mixers, and microfluidic baffle mixers can also be used. After the LNPs are formed they are diluted with buffer, for example phosphate, HEPES, or Tris, in a pH range of 6 to 8.5 to reduce the alcohol (ethanol) concentration, The diluted LNPs are purified either by dialysis or ultrafiltration or diafiltration using tangential flow filtration (TFF) against a buffer in a pH range of 6 to 8.5 (for example, phosphate, HEPES, or Tris) to remove the alcohol. Alternatively, one can use size exclusion chromatography. Once the alcohol is completely removed the buffer is exchanged with like buffer containing a cryoprotectant (for example, glycerol or a sugar such as sucrose, trehalose, or mannose). The LNPs are concentrated to a desired concentrated, followed by 0.2 μm filtration through, for example, a polyethersulfone (PES) or modified PES filter and filled into glass vials, stoppered, capped, and stored frozen. In alternative embodiments, a lyoprotectant is used and the LNP lyophilized for storage instead of as a frozen liquid. Further methodologies for making LNP can be found, for example, in U.S. Patent Application Publication Nos. US 2020/0297634, US 2013/0115274, and International Patent Application Publication No. WO 2017/048770, each of which is incorporated by reference for all that they teach about the production of LNP.

Some aspects are a method of making a tLNP comprising rapid mixing of an aqueous solution of a nucleic acid (or other negatively charged payload) and an alcoholic solution of the lipids as disclosed for LNP. In some embodiments, the lipid mixture includes functionalized PEG-lipid, for later conjugation to a targeting moiety. As used herein, functionalized PEG-lipid refers to a PEG-lipid in which the PEG moiety has been derivatized with a chemically reactive group (such as, maleimide, N-hydroxysuccinimide (NHS) ester, Cys, azide, alkyne, and the like) that can be used for conjugating a targeting moiety to the PEG-lipid, and thus, to the LNP comprising the PEG-lipid. In other embodiments, the functionalized PEG-lipid is inserted into and LNP subsequent to initial formation of an LNP from other components. In either type of embodiment, the targeting moiety is conjugated to functionalized PEG-lipid after the functionalized PEG-lipid containing LNP is formed. Protocols for conjugation can be found, for example, in Parhiz et al. 2018, J. Controlled Release 291:106-115, and Tombacz et al., 2021, Molecular Therapy 29(11):3293-3304, each of which is incorporated by reference for all that it teaches about conjugation of PEG-lipids to binding moieties. Alternatively, the targeting moiety can be conjugated to the PEG-lipid prior to insertion into pre-formed LNP.

In certain embodiments of the preparation methods of tLNP, the method comprises:
i) forming an initial LNP by mixing all components of the tLNP, in proportions disclosed herein, except for the one or more functionalized PEG-lipids and the one or more targeting moieties;
ii) forming a pre-conjugation tLNP by mixing the initial LNP with the one or more functionalized PEG-lipids; and
iii). forming the tLNP by conjugating the pre-conjugation tLNP with the one or more targeting moieties.

In certain embodiments of the preparation methods of tLNP, the method comprises:
i) forming a pre-conjugation tLNP by mixing all components of the tLNP, in proportions disclosed herein, including the one or more functionalized PEG-lipids, except for the one or more targeting moieties; and
ii) forming the tLNP by conjugating the pre-conjugation tLNP with the one or more targeting moieties.

In certain embodiments of the preparation methods of tLNP, the method comprises:
i) forming one or more conjugated functionalized PEG-lipids by conjugating the one or more functionalized PEG-lipids with the one or more targeting moieties; and
ii) forming the tLNP by mixing all components of the tLNP, in proportions disclosed herein, including the one or more conjugated functionalized PEG-lipids.

In certain embodiments of the preparation methods of tLNP, the method comprises:
i) forming one or more conjugated functionalized PEG-lipids by conjugating the one or more functionalized PEG-lipids with the one or more targeting moieties;
ii) forming an LNP by mixing all components of the tLNP, except the one or more conjugated functionalized PEG-lipids; and
iii) forming the tLNP by mixing the initial LNP with the one or more conjugated functionalized PEG-lipids.

After the conjugation the tLNPs are purified by dialysis, tangential flow filtration, or size exclusion chromatography, and stored, as disclosed above for LNPs.

The encapsulation efficiency of the nucleic acid by the LNP or tLNP is typically determined with a nucleic acid binding fluorescent dye added to intact and lysed aliquots of the final LNP or tLNP preparation to determine the amounts of unencapsulated and total nucleic acid, respectively. Encapsulation efficiency is typically expressed as a percentage and calculated as 100×(T−U)/T where T is the total amount of nucleic acid and U is the amount of unencapsulated nucleic acid. In various embodiments, the encapsulation efficiency is ≥80%, ≥85%, ≥90%, or ≥95%.

(VI) Methods of Delivering mRNA into a Cell

In other aspects, disclosed herein are methods of delivering an RNA into a cell comprising contacting the cell with LNP or tLNP disclosed herein. Accordingly, each of the herein disclosed genera, subgenera, and or species of LNP or tLNP disclosed herein including those based on the inclusion or exclusion of particular lipids, particular lipid compositions, particular payloads, and/or particular targeting moieties can be used in defining the scope of the methods of delivering a payload to a cell. In some embodiments the contacting takes place ex vivo. In some embodiments, the contacting takes place extracorporeally. In some embodiments, the contacting takes place in vivo. In some embodiments, an LNP or tLNP is contacted with target cells in vivo, by systemic or local administration. In some embodiments, the in vivo contacting comprises intravenous, intramuscular, subcutaneous, intralesional, intranodal or intralymphatic administration. In some embodiments, administration is by intravenous or subcutaneous infusion or injection. In some embodiments, administration is by intraperitoneal or intralesional infusion injection. In further instances, transfection of hepatocytes is reduced as compared to tLNPs comprising a conventional ionizable cationic lipid, such as ALC-0315. In some embodiments, an LNP or tLNP is administered 1-3 times a week for 1, 2, 3, or 4 weeks. In some embodiments, toxicity is confined (or largely confined) to grades of 0 or 1 or 2, as discussed above.

The herein disclosed LNP and tLNP compositions and formulations have reduced toxicity as compared to widely used prior art LNP compositions such as those containing ALC-0315. In various embodiments the toxicity can be described as an observable toxicity, a substantial toxicity, a severe toxicity, or an acceptable toxicity, or a dose-limiting toxicity (such as but not limited to a maximum tolerated dose (MTD)). By an observable toxicity it is meant that while a change is observed the effect is negligible or mild. By substantial toxicity it is meant that there is a negative impact on the patient's overall health or quality of life. In some instances, a substantial toxicity can be mitigated or resolved with other ongoing medical intervention. By a severe toxicity it is meant that the effect requires acute medical intervention and/or dose reduction or suspension of treatment. The acceptability of a toxicity will be influenced by the particular disease being treated and its severity and the availability of mitigating medical intervention. In some embodiments, toxicity is confined (or largely confined) to an observable toxicity. In some embodiments, toxicity is confined (or largely confined) to grades of 0 or 1 or 2.

In some embodiments, the method of delivering is a method of transfecting. In some embodiments, the mRNA encodes an immune receptor or immune cell engager and the method of delivering is also a method of reprogramming an immune cell. In some embodiments, the mRNA encodes, or is, a BRM and the method of delivering is also a method of providing a conditioning agent. In various embodiments, the BRM or conditioning agent is a gamma chain receptor cytokine such as IL-2, IL-7, IL-15, IL-15/15Ralpha, IL-21; an immune modulating cytokine such as IL-12, IL-18; a chemokine such as RANTES, IP10, MIG; or another BRM such as Flt3, GM-CSF, and G-CSF.

In some embodiments, the mRNA encodes a gene/genome editing enzyme and/or a guide RNA or other component of a gene/genome editing system and the method of delivering is also a method of reprogramming a cell. In some instances, the cell is an immune cell. In some instances, the cell is an HSC. In some instances, the cell is an MSC.

In certain embodiments comprising delivering the mRNA into an immune cell, the binding moiety binds to a lymphocyte surface molecule or a monocyte surface molecule. Lymphocyte surface molecules include CD2, CD3, CD4, CD5, CD7, CD8, CD28, 4-1BB (CD137), CD166, CTLA-4, OX40, PD-1, GITR, LAG-3, TIM-3, CD25, low affinity IL-2 receptor, IL-7 receptor, IL-12 receptor, IL-15 receptor, IL-18 receptor, and IL-21 receptor. Monocyte surface molecules include CD5, CD14, CD16a, CD32, CD40, CD11b (Mac-1), CD64, DEC205, CD68, and TREM2. Exemplary antibodies that can provide antigen binding domains to bind these surface molecules are disclosed herein. Such antibodies, individually and collectively, constitute means for binding to an immune cell (or leukocyte)—or to a lymphocyte or monocyte, as indicated.

In certain embodiments comprising delivering the mRNA into a stem cell, the binding moiety binds to a HSC surface molecule or a MSC surface molecule. HSC surface molecules include CD117, CD34, CD44, CD90 (Thy1), CD105, CD133, BMPR2, and Sca-1. MSC surface molecules include CD70, CD105, CD73, Stro-1, SSEA-4, CD271, CD146, GD2, SSEA-3, SUSD2, Stro-4, MSCA-1, CD56, CD200, PODXL, CD13, CD29, CD44, and CD10. Exemplary antibodies that can provide antigen binding domains to bind these surface molecules are disclosed herein above. Such antibodies, individually and collectively, constitute means for binding to a stem cell—or to an HSC or MSC, as indicated.

Other delivery vehicles than LNP and tLNP can be used to deliver a herein disclosed improved mRNA into a cell. In some alternatives, the improved mRNA is encapsulated in a polymeric nanoparticle or mixed lipid-polymer nanoparticle. In other alternatives, the improved mRNA or a nucleic acid encoding the improved mRNA is packaged in a viral particle, such as, but not limited to, a lentiviral, retroviral, adenoviral, adeno-associated viral, anelloviral, or vaccinia viral vector.

(VII) Methods of Treatment with mRNA

In certain other aspects, this disclosure provides a method of treating a disease or disorder comprising administering an improved mRNA disclosed herein or a pharmaceutical composition of an improved mRNA disclosed herein to a subject in need thereof. In alternative embodiments, a herein disclosed improved mRNA or pharmaceutical composition comprising the improved mRNA is used to transfect a cell ex vivo or extracorporeally, and the transiently transfected cell is administered to the subject in need thereof. In still further alternatives, the improved mRNA or a nucleic acid encoding the improved mRNA is packaged in a viral particle and the viral particle is used to transfect a cell ex vivo or extracorporeally, and the transduced cell is administered to the subject in need thereof. In various embodiments the disease or disorder is cancer or a genetic disease or an autoimmune or fibrotic disease. In some embodiments, the subject is administered a pharmacologically effective dose. In some embodiments, the subject is administered a therapeutically effective dose. In some embodiments, the subject is a mammal. In some embodiments, the mammalian subject is a human. In some embodiments, administration is by intravenous, subcutaneous, intraperitoneal, or intralesional infusion or injection.

In some embodiments, the improved mRNA encoding a chimeric antigen receptor (CAR) enclosed in a tLNP. The receptors are chimeric because they combine both antigen-binding and T cell activating functions into a single receptor. In some embodiments, one or more mRNA species encoding one or more CARs; for example, a single or multiple species of mRNA encoding a single CAR species, or multiple species of mRNA encoding multiple CAR species. In some instances, these multiple CAR species have a same specificity while in other instances they have multiple specificities. In some embodiments, a CAR of this disclosure is multispecific, for example, bispecific, comprising multiple antigen binding moieties each specific for separate antigens. For example, The CAR in LCAR-AIO targets three antigens—CD19, CD20 and CD22 (Zhou et al., Blood (2021) 138 (Supplement 1): 1700).

In certain embodiments, CARs are used to treat a disease or condition associated with a target cell that expresses the antigen targeted by the CAR. For example, in some embodiments, an anti-CD19 or anti-CD20 CAR can be used to target and treat B cell malignancies or B cell-mediated autoimmune conditions or diseases (e.g., having an immune cell targeting moiety, such as an anti-CD8 antibody). In other embodiments, an anti-FAP CAR can be used to target and treat solid tumors or fibrosis (e.g., cardiac fibrosis, cancer-associated fibroblasts), which can also have an immune cell targeting moiety, such as an anti-CD8 antibody. Examples of CARs that can be used in accordance with the embodiments described herein include to those disclosed in U.S. Pat. No. 7,446,190 (anti-CD19), U.S. Pat. No. 10,287,35 (anti-CD19), US2021/0363245 (anti-CD19 and anti-CD20), U.S. Pat. No. 10,543,263 (anti-CD22), U.S. Pat. No. 10,426,797 (anti-CD33), U.S. Pat. No. 10,844,128 (anti-CD123), U.S. Pat. No. 10,428,141 (anti-ROR1), WO2022247756, WO2020148677, WO2020092854, & US20230331872 (anti-GPRC5D), WO2016090337, WO2022263855, & WO2024047558 (anti-FCRL5), and US2021/0087295 (anti-FAP), each of which is incorporated by reference for all that it teaches about CAR structure and function generically and with respect to the CAR's antigenic specificity and target indications to the extent that it is not inconsistent with the present disclosure. Each CAR constitutes means for targeting an immune cell, for example, a T cell, to the indicated antigen.

Exemplary target antigens against which a CAR, TCR, or ICE are described in (II)(d)(2)(2b). In some embodiments, the tLNP comprises an mRNA encoding an anti-CD19 chimeric antigen receptor (CAR). Examples of anti-CD19 CARs, anti-CD20 CARs, anti-BCMA CARs, anti-GPRC5D CAR, and anti-FCRL5 CAR are described in part (III)(d)(2)(2f) above.

In some embodiments, the tLNP comprises one or more mRNA encoding one or more CARs that target multiple antigens. In some embodiments, the tLNP comprises distinct mRNAs that are encapsulated together in a single tLNP, with each mRNA encoding one monospecific CAR. For examples, the tLNP can comprise an mRNA encoding an anti-CD19 CAR and an mRNA encoding an anti-CD20 CAR, an mRNA encoding an anti-CD19 CAR and an mRNA encoding an anti-BCMA CAR, an mRNA encoding an anti-GPRC5D CAR and an mRNA encoding an anti-BCMA CAR, or an mRNA encoding an anti-FCRL5 CAR and an mRNA encoding an anti-BCMA CAR. In some embodiments, the tLNP comprises a single mRNA encoding a bicistronic mRNA encoding two monospecific CARs. For example, the bicistronic mRNA can encode an anti-CD19 CAR and an anti-CD20 CAR, an anti-CD19 CAR and an anti-BCMA CAR, an anti-GPRC5D CAR and an anti-BCMA CAR, or an anti-FCRL5 CAR and an anti-BCMA CAR. In further embodiments, the tLNP comprises a single mRNA encoding a multicistronic mRNA encoding more than two monospecific CARs. In some embodiments, the tLNP comprises a single mRNA encoding an mRNA encoding a multispecific CAR. In some embodiments, the tLNP comprises a single mRNA encoding an mRNA encoding a bispecific CAR. For example, the mRNA can encode an anti-CD19 and anti-CD20 bispecific CAR, an anti-CD19 and anti-BCMA bispecific CAR, an anti-GPRC5D and anti-BCMA bispecific CAR, or an anti-FCRL5 and anti-BCMA bispecific CAR. In some embodiments, multiple tLNPs can be co-formulated in a combination with each tLNP comprising one mRNA. In some instances, the one mRNA encodes one monospecific CAR. For example, two tLNPs can be co-formulated with one tLNP comprising an mRNA encoding an anti-CD19 CAR and the other tLNP comprising an mRNA encoding an anti-CD20 CAR, one tLNP comprising an mRNA encoding an anti-CD19 CAR and the other tLNP comprising an mRNA encoding an anti-BCMA CAR, one tLNP comprising an mRNA encoding an anti-GPRC5D CAR and the other tLNP comprising an mRNA encoding an anti-BCMA CAR, or one tLNP comprising an mRNA encoding an anti-FCRL5 CAR and the other tLNP comprising an mRNA encoding an anti-BCMA CAR. In some embodiments, multiple tLNPs can be co-administered in a combination, either simultaneously or sequentially, wherein each comprises one mRNA. In some instances, the one mRNA encodes one monospecific CAR. For example, two tLNPs can be co-administered in a combination, either simultaneously or sequentially, with one tLNP comprising an mRNA encoding an anti-CD19 CAR and the other tLNP comprising an mRNA encoding an anti-CD20 CAR, one tLNP comprising an mRNA encoding an anti-C19 CAR and the other tLNP comprising an mRNA encoding an anti-BCMA CAR, one tLNP comprising an mRNA encoding an anti-GPRC5D CAR and the other tLNP comprising an mRNA encoding an anti-BCMA CAR, or one tLNP comprising an mRNA encoding an anti-FCRL5 CAR and the other tLNP comprising an mRNA encoding an anti-BCMA CAR. The targeting can be mediated by any of the CARs described herein. In addition to combinations of two specificities, higher order combinations are also possible, especially with the use of bi- and tri-specific CARs. Following these patterns, further embodiments are constituted mutatis mutandis by other tLNP or combinations of tLNPs comprising one or more mRNAs encoding one or more CARs that target multiple antigens involving these and other CAR specificities disclosed herein.

Cellular therapy involving the administration of genetically engineered cells to a patient has generally required depleting or ablative conditioning to facilitate engraftment of the engineered cells (for example, T cells or HSC). In the context of in vivo engineering and reprogramming such conditioning would be counterproductive as the conditioning would eliminate the very cells that are to be engineered. Instead, one can utilize activating and/or adjuvant conditioning to increase the number of cells amenable to engineering, to mobilize them to the locus of pathology, to make the locus of pathology (for example, a tumor microenvironment) more susceptible to treatment, to augment the therapeutic effect, etc., as appropriate for the particular disease and primary treatment. Conditioning agents include biological response modifiers (BRMs) that can be delivered directly to a subject or encoded in an mRNA, and delivered to a subject using the LNP and tLNP compositions and formulations disclosed herein.

Accordingly, certain aspects are methods of conditioning a subject who receives an engineering agent comprising providing a tLNP comprising an mRNA encoding a conditioning agent to the subject prior to, concurrently with, or subsequent to administration of the engineering agent. In various embodiments, an encoded conditioning agent comprises a γ-chain receptor agonist, an inflammatory chemokine, a pan-activating cytokine, an antigen presenting cell activity enhancer, an immune checkpoint inhibitor, or an anti-CCR4 antibody. In some embodiments, the γ-chain receptor cytokine comprises IL-15, IL-2, IL-7, or IL-21. In some embodiments, the immune checkpoint inhibitor comprises an anti-CTLA-4, anti-PD-1, anti-PD-L1, anti-Tim-3, or anti-LAG-3 antibody. In some embodiments, the inflammatory chemokine comprises CCL2, CCL3, CCL4, CCL5, CCL11, CXCL1, CXCL2, CXCL-8, CXCL9, CXCL10, or CXCL11. In some embodiments, the antigen presenting cell activity enhancer comprises Flt-3 ligand, gm-CSF, or IL-18. In some embodiments, a pan-activating cytokine comprises IL-12 of IL 18. In certain embodiments, a conditioning agent comprises a transcription factor, for example, one selected from the group consisting of nuclear factor of activated T-cells (NFAT), NF-κB, T-bet, signal transducer and activator of transcription 4 (STAT4), Blimp-1, c-Jun, and Eomesodermin (Eomes) and the tLNP is targeted to a T cell. In some embodiments, a tLNP encapsulating the mRNA-encoded conditioning agent is administered systemically, for example, by intravenous or subcutaneous infusion or injection. In other embodiments, the tLNP is administered locally, for example, by intralesional or intraperitoneal injection or infusion. In some embodiments, mRNA encoding the conditioning agent and the engineering agent are encapsulated in the same tLNP while in other embodiments they are encapsulated in separate tLNPs. These two modes of delivery of conditioning agents are described in greater detail in PCT application PCT/US 2023/072426, which is incorporated by reference for all that it teaches about conditioning agents and their delivery of LNPs or tLNPs that is not inconsistent with the present disclosure.

The term “treating” or “treatment” broadly includes any kind of treatment activity, including mitigation, cure or prevention of disease, or an aspect thereof, in man or other animals, or any activity that otherwise affects the structure or any function of the body of man or other animals. Treatment activity includes administration of the medicaments, dosage forms, and pharmaceutical compositions described herein to a patient, especially according to the various methods of treatment disclosed herein, whether by a healthcare professional, the patient his/herself, or any other person.

Some embodiments of these methods of treatment comprise administration of an effective amount of an improved mRNA or a composition disclosed herein. Some instances relate to a therapeutically (or prophylactically) effective amount. A therapeutically effective amount is not necessarily a clinically effective amount, that is, while there can be therapeutic benefit as compared to no treatment, a method of treatment may not be equivalent or superior to a standard of care treatment existing at some point in time. Other instances relate to a pharmacologically effective amount, that is an amount or dose that produces an effect that correlates with or is reasonably predictive of therapeutic (or prophylactic) utility. As used herein, the term “therapeutically effective amount” is synonymous with “therapeutically effective dose” and means at least the minimum dose of an improved mRNA or composition disclosed herein necessary to achieve the desired therapeutic or prophylactic effect. Similarly, a pharmacologically effective dose means at least the minimum dose of an improved mRNA or composition disclosed herein necessary to achieve the desired pharmacologic effect. Some embodiments refer to an amount sufficient to prevent or disrupt a disease process, deplete or ablate a cell population, or to reduce the extent or duration of pathology. Some embodiments refer to a dose sufficient to reduce a symptom associated with the disease or condition being treated. An effective dosage or amount of an improved mRNA or a composition disclosed herein can readily be determined by the person of ordinary skill in the art considering all criteria (for example, the rate of excretion of the compound or composition used, the pharmacodynamics of the improved mRNA or composition used, the nature of the other compounds to be included in the composition, the particular route of administration, the particular characteristics, history and risk factors of the individual, such as, e.g., age, weight, general health and the like, the response of the individual to the treatment, or any combination thereof) and utilizing his/her best judgment on the individual's behalf. Exemplary dosages are also disclosed in the Examples herein below.

In further embodiments, a tLNP can be administered in combination with the standard of care for a particular indication, such as corticosteroids (e.g., prednisone) for management of myositis or lupus nephritis. In certain cases, myositis is also treated with methotrexate, which can be combined with immunosuppressive agents (e.g., azathioprine, mycophenolate mofetil, tacrolimus), which are usually required in addition to corticosteroids. For membranous nephropathy, cyclical steroids and cyclophosphamide might be used in combination with tLNPs of this disclosure. In other cases, an anti-IL-6, such as tocilizumab, can also be used as a pretreatment or in combination with tLNPs of this disclosure. These combinations can be administered concurrently or sequentially.

In some embodiments, the disease or disorder is an autoimmune disease. Examples of autoimmune disease include, without limitation, myocarditis, acute idiopathic thrombocytopenic purpura, chronic idiopathic thrombocytopenic purpura, dermatomyositis, Sydenham's chorea, myasthenia gravis, systemic lupus erythematosus, anti-neutrophilic cytoplasmic antibody (ANCA) vasculitis, fibrosing alveolitis, multiple sclerosis, rheumatic fever, polyglandular syndromes, agranulocytosis, autoimmune hemolytic anemias, bullous pemphigoid, Wegener's granulomatosis, membranous nephropathy, amyotrophic lateral sclerosis, tabes dorsalis, giant cell arteritis/polymyalgia, pernicious anemia, rapidly progressive glomerulonephritis, IgA nephropathy, polyarteritis nodosa, ankylosing spondylitis, allergic responses, insulin-resistant diabetes, psoriasis, diabetes mellitus, Addison's disease, Grave's disease, diabetes, endometriosis, celiac disease, Crohn's disease, Henoch-Schonlein purpura, ulcerative colitis, Goodpasture's syndrome, thromboangitis obliterans, Sjögren's syndrome, aplastic anemia, rheumatoid arthritis, sarcoidosis, scleritis, a T cell-mediated autoimmunity or a B cell-mediated autoimmunity, a B cell-mediated autoimmune disease, immune-mediated necrotizing myopathy (IMNM), chronic inflammatory demyelinating polyneuropathy (CIDP), myeloid oligodendrocyte glycoprotein autoantibody associated disease (MOGAD), amyloid light-chain amyloidosis, neuromyelitis optica (NMO) myositis, neuromyelitis optica spectrum disorder (NMOSD), pemphigus vulgaris, polymyositis, dermatomyositis, immune mediated necrotizing myopathy, systemic sclerosis, anti-synthetase syndrome (idiopathic inflammatory myopathy), stiff person syndrome, lupus nephritis, membranous nephropathy, severe combined immunodeficiency, Fanconi anemia, and vasculitis.

In some embodiments, the autoimmune disease is a T cell-mediated autoimmunity or a B cell-mediated autoimmunity. In some instances, the B cell-mediated autoimmune disease is myositis (such as anti-synthetase myositis), lupus nephritis, membranous nephropathy, systemic lupus erythematosus, anti-neutrophilic cytoplasmic antibody (ANCA) vasculitis, neuromyelitis optica spectrum disorder (NMOSD), myasthenia gravis, pemphigus vulgaris, polymyositis, dermatomyositis, immune mediated necrotizing myopathy, systemic sclerosis, diffuse cutaneous systemic sclerosis, limited cutaneous systemic sclerosis, anti-synthetase syndrome (idiopathic inflammatory myopathy), rheumatoid arthritis, stiff person syndrome, myeloid oligodendrocyte glycoprotein autoantibody associated disease (MOGAD), amyloid light-chain amyloidosis, immune-mediated necrotizing myopathy (IMNM), multiple sclerosis, primary progressive multiple sclerosis, relapsing-remitting multiple sclerosis, secondary progressive multiple sclerosis, non-active secondary progressive multiple sclerosis, Sjögren's syndrome, IgA nephropathy, severe combined immunodeficiency, or Fanconi anemia. In certain embodiments, the B cell-mediated autoimmune disease is myositis, lupus nephritis, membranous neuropathy, scleroderma, systemic lupus erythematosus, myasthenia gravis, ANCA vasculitis, multiple sclerosis, or pemphigus vulgaris. In certain embodiments, the B cell-mediated autoimmune disease is myositis, lupus nephritis, membranous neuropathy, or scleroderma. In certain embodiments, the B cell-mediated autoimmune disease is myositis. In some instances, the myositis is anti-synthetase myositis. In certain embodiments, the B cell-mediated autoimmune disease is systemic lupus erythematosus, myasthenia gravis, ANCA vasculitis, multiple sclerosis, or pemphigus vulgaris.

In some embodiments, the disease or disorder is rejection of an allogeneic organ or tissue graft. Pre-existing antibodies and/or B cells, in their role as antigen presenting cells, can facilitate rapid immune rejection through known mechanisms hence depleting a large number of B cells can help prevent allograft rejection.

In some embodiments, the disease or disorder is a cancer. Examples of cancers include, without limitation, carcinomas, sarcomas, and hematologic cancers. In some embodiments, the hematologic cancer is a lymphoma, leukemia, or myeloma. In some instances, the hematologic cancer is a B lineage or T lineage cancer. In some instances, the B lineage cancer is multiple myeloma, diffuse large B cell lymphoma, acute myeloid leukemia, Mantle Cell lymphoma, follicular lymphoma, B acute lymphoblastic leukemia, chronic lymphocytic leukemia, or myelodysplastic syndrome. In some embodiments, the cancer is a sarcoma. In some embodiments, the cancer is a carcinoma, such as breast cancer, colon cancer, ovarian cancer, lung cancer, testicular cancer, or pancreatic cancer. In some embodiments, the cancer is melanoma.

In some embodiments, the disease or disorder is a fibrotic disease or disorder. In some instances, the fibrotic disease is cardiac fibrosis, arthritis, idiopathic pulmonary fibrosis, and nonalcoholic steatohepatitis (also known as metabolic dysfunction-associated steatohepatitis). In other instances, the disorder involves tumor-associated fibroblasts.

In some embodiments, the disease or disorder is a genetic disease or disorder such as a monogenic genetic disease. In some instances, the genetic disease or disorder is a hemoglobinopathy, for example, sickle cell disease or β-thalassemia.

In certain other aspects, this disclosure provides a method of depleting CD19+ cells in vivo comprising administering a tLNP encapsulating an mRNA encoding a CAR comprising an anti-human CD19 scFv or VHH. In some embodiments, the CAR comprising an anti-human CD19 scFv comprises the variable domains of monoclonal antibody 47G4, a CD28 hinge/transmembrane, a CD28 costimulatory domain, and a CD3ζ signaling domain to a mammalian subject, wherein the tLNP is targeted to CD2, CD5 or CD8, whereby CD2+, CD5+, or CD8+ cells, respectively, are transfected in vivo to express the mRNA. In some embodiments, the CD19+ cell is a normal B cell. In some embodiments, the CD19+ cell is a normal short-lived plasma cell. In some embodiments, the CD19+ cell is a tumor cell.

In certain embodiments, a greater proportion of CD19+ cells are ablated (depleted) as compared to a subject administered a same dosage of tLNP encapsulating an otherwise identical mRNA encoding a CAR comprising an anti-human CD19 scFv or VHH comprising the variable domains of monoclonal antibody 47G4, a CD8α hinge/transmembrane domains, a CD28 costimulatory domain, and a CD3ζ signaling domain.

In certain embodiments, the CAR comprising an anti-human CD19 scFv comprising the variable domains of monoclonal antibody 47G4, a CD28 hinge/transmembrane, a CD28 costimulatory domain, and a CD3ζ signaling domain.

In certain other aspects, this disclosure provides a method of depleting CD20+ cells in vivo comprising administering a tLNP encapsulating an mRNA encoding a CAR comprising an anti-human CD20 scFv or VHH. In some embodiments, the CAR comprising an anti-human CD20 scFv comprises the variable domains of monoclonal antibody Leu16 or 2.1.2, a CD28 hinge/transmembrane, a CD28 costimulatory domain, and a CD3ζ signaling domain to a mammalian subject, wherein the tLNP is targeted to CD2, CD5 or CD8, whereby CD2+, CD5+, or CD8+ cells, respectively, are transfected in vivo to express the mRNA. In alternative embodiments, the anti-CD20 CAR comprises an IgG4 Fc hinge. In alternative embodiments, the anti-CD20 CAR comprises a 4-1 BB or 4-1 BB+CD28 co-stimulatory domain. In some embodiments, the CD20+ cell is a normal B cell. In some embodiments, the CD20+ cell is a tumor cell.

In certain embodiments, a greater proportion of CD20+ cells are depleted as compared to a subject administered a same dosage of tLNP encapsulating an otherwise identical mRNA encoding a CAR comprising an anti-human CD20 scFv or VHH comprising CD8α hinge/transmembrane domains.

In certain other aspects, this disclosure provides a method of depleting BCMA+ cells in vivo comprising administering a tLNP encapsulating an mRNA encoding a CAR comprising an anti-human BCMA scFv or VHH. In some embodiments, the CAR comprising an anti-human BCMA scFv comprises a CD28 hinge/transmembrane, a CD28 costimulatory domain, and a CD3ζ signaling domain to a mammalian subject, wherein the tLNP is targeted to CD2, CD5 or CD8, whereby CD2+, CD5+, or CD8+ cells, respectively, are transfected in vivo to express the mRNA. In alternative embodiments, the anti-BCMA CAR comprises an IgG4 Fc hinge. In alternative embodiments, the anti-BCMA CAR comprises a 4-1BB or 4-1BB+CD28 co-stimulatory domain. In some embodiments, the BCMA+ cell is a normal B cell. In some embodiments, the BCMA+ cell is a tumor cell.

In certain embodiments, a greater proportion of BCMA+ cells are depleted as compared to a subject administered a same dosage of tLNP encapsulating an otherwise identical mRNA encoding a CAR comprising an anti-human BCMA scFv or VHH comprising CD8α hinge/transmembrane domains.

Individually and collectively, methods of depleting CD19+, CD20+, and/or BCMA+ cells can be termed methods of depleting B cells.

In certain other aspects, this disclosure provides a method of transiently transfecting a cell in vivo comprising administering a pharmaceutical composition of the improved mRNA disclosed herein to a mammalian subject. In certain embodiments, the mammalian subject is a human subject.

In certain embodiments, assessment of effectiveness of tLNP encapsulating mRNA encoding CAR is measured by elimination of B cells in a human, for example, a healthy normal volunteer, a cancer patient, or a B cell-mediated autoimmune disease patient.

Unless otherwise defined, all technical terms used herein have the same meaning as commonly understood by a person of ordinary skill in the art to which this disclosure belongs.

For the purposes of promoting an understanding of the principles of the disclosure, reference will now be made to embodiments and specific language will be used to describe the same. It will nevertheless be understood that no limitation of the scope of the disclosure was thereby intended, such alteration and further modifications of the disclosure as illustrated herein, being contemplated as would normally occur to one skilled in the art to which the disclosure relates.

Various exemplary embodiments of compositions and methods according to this invention are now described in the following non-limiting Examples. The Examples are offered for illustrative purposes only and are not intended to limit the scope of this invention in any way. Indeed, various modifications of the invention in addition to those shown and described herein will become apparent to those skilled in the art from the foregoing description and the following examples and fall within the scope of the appended claims.

### EXAMPLES

The following examples are intended to illustrate various embodiments of the invention. Accordingly, the specific embodiments discussed are not to be constructed as limitations on the scope of the invention. It will be apparent to one skilled in the art that various equivalents, changes, and modifications can be made without departing from the scope of invention, and it is understood that such equivalent embodiments are to be included herein. Further, all references cited in the disclosure are hereby incorporated by reference in their entirety, as if fully set forth herein, to the extent that they do not contradict or are not inconsistent with the instant disclosure. Some examples make use of donor material and donors are numbered separately in each Example so that Donor 1 in one Example is not necessarily the same as Donor 1 or different than Donor 2 of another Example, unless explicitly stated otherwise. DNA sequences written with thymine bases should be understood to also describe RNA molecules and sequences written with uracil bases, and vice versa.

### (1) Methods:

1) mRNA preparation: Two CAR configurations were used for anti-CD19 CAR: CAR1 and CAR2 (Brudno et al., 2020, Nat Med 26(2):270-280). The CAR1 mRNAs encode an amino acid sequence (SEQ ID: 1, FIG. 2A) as shown in FIG. 1. The CAR1 amino acid sequence was originally disclosed in U.S. Pat. No. 10,287,350 (WO2015/187528) as SEQ ID NO: 1, from which the CAR1 amino acid sequence and its synthesis are incorporated herein by reference. The amino acid sequence of the mature CAR1 protein (i.e., without a signal peptide) is provided as SEQ ID NO: 4 (Table 1). The CAR2 mRNAs encode an amino acid sequence (SEQ ID NO: 2, FIG. 2A) as shown in FIG. 1. The amino acid sequence of the mature CAR2 protein (i.e., without a signal peptide) is provided as SEQ ID NO: 5 (FIG. 2B). Combining the 47G4 scFv as well as the CD28 hinge and transmembrane domains provides CAR2 an advantage for transient in vivo transfection as opposed to the traditional CAR-T cell comprising an integrated DNA sequence encoding CAR. CAR2 is expressed at a higher level than CAR1 from mRNAs using the same UTRs and codon optimization method and the T cells expressing CAR2 eliminate more CD19⁺ cells. CAR25 was provided herein as a CAR configuration used for anti-CD20 CAR. The CAR25 mRNA encodes an amino acid sequence (SEQ ID: 3, FIG. 2A) as shown in FIG. 1A. The amino acid sequence of the mature CAR25 protein (i.e., without a signal peptide) is provided as SEQ ID NO: 6 (FIG. 2B).

### 1a) UTRs and ORF

The mRNA sequences used for mRNA improvement for CAR1, CAR2, and CAR25 comprise in 5′ to 3′ order, a 5′ untranslated region (UTR), an open reading frame (ORF) comprising a RNA sequence that encodes the corresponding CAR, at least one stop codon, a 3′ UTR, and an encoded polyadenine (poly(A)) sequence.

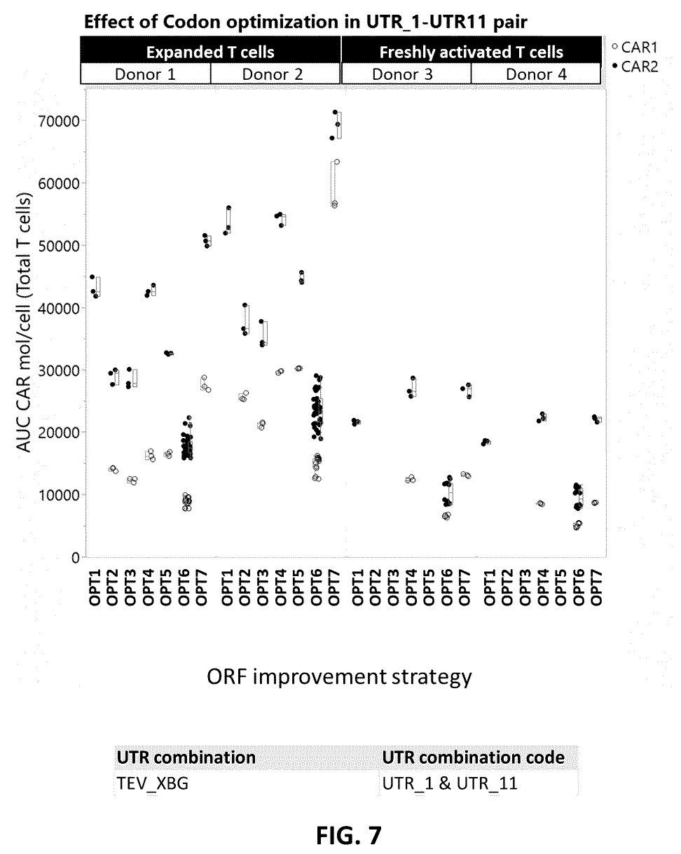

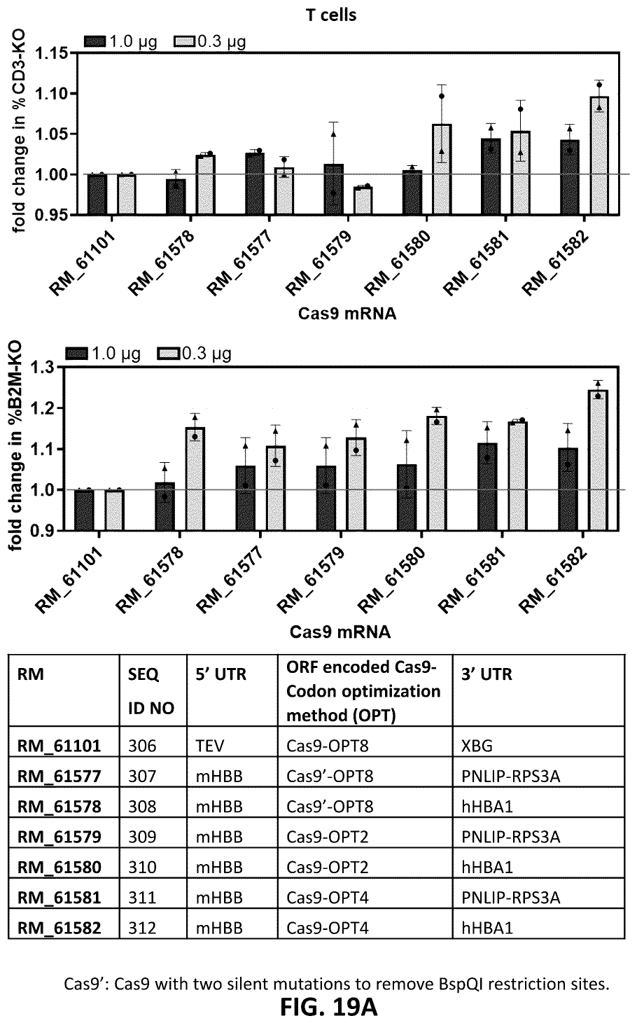

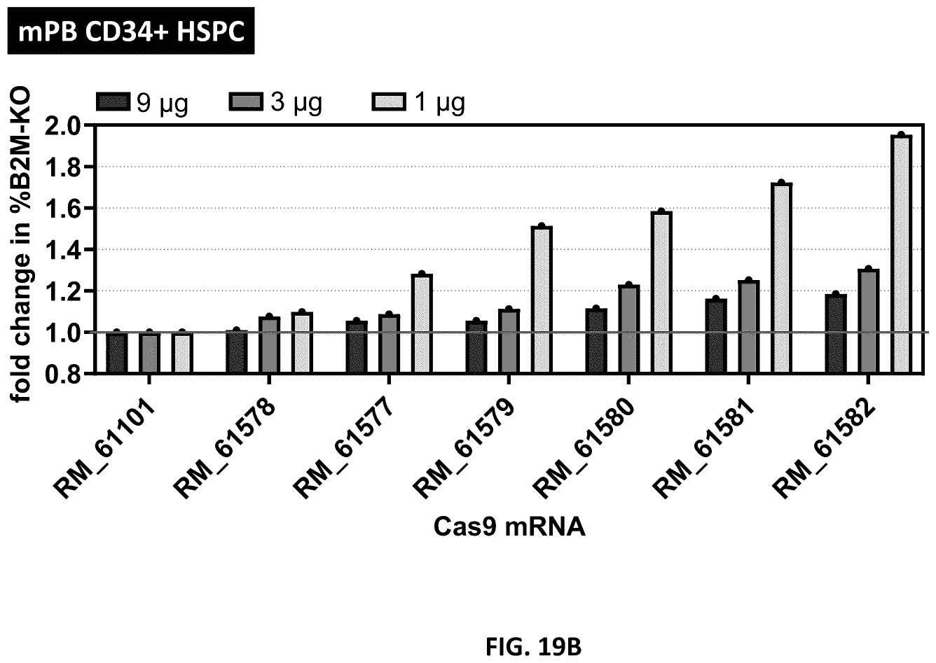

The mRNA screening for CAR1 and CAR2 described in Examples 1-5 used the 5′ UTR sequences are summarized in the table of FIG. 8A and FIG. 8B, the 3′ UTR sequences are summarized in the table of FIG. 8A and FIG. 8C, and the ORF sequences are summarized in FIG. 7B, FIG. 19A, and FIG. 19B. The mRNA sequences generated for mRNA screening in Example 3 are summarized in Table 15. The most commonly used 5′ UTR, ORF, and 3′ UTR have an additional code designation summarized in FIG. 3C. Several strategies to codon optimize the ORF are summarized in Table 1.

Each 5′ UTR present within the mRNA in this Example contained an AGCAUAAAAG sequence (SEQ ID NO: 7) on the 5′ end of the 5′ UTR, facilitating co-transcriptional capping with the cap-AG reagent. Use of other co-transcriptional capping reagents or post-transcriptional capping can be facilitated by or permissive of other 5′ terminal sequences. The unstructured portion of the previous sequence improved cap recognition. The 5′ UTR each also contained a GAAUUCGCUGCCACC sequence (SEQ ID NO: 8) at the 3′ end of the 5′ UTR to incorporate an EcoR1 restriction enzyme recognition site and a Kozak sequence.

Each 3′ UTR present within the mRNA in this Example contained an AGGAUCC sequence (SEQ ID NO: 9) on the 5′ end of the 3′ UTR, incorporating the first adenosine to improve translational stop efficiency and a BamH1 restriction enzyme recognition site. The sequence UGUACA (SEQ ID NO: 10) present in most constructs was located on the 3′ end of each 3′ UTR (before the poly(A)), to provide a BsrG1 restriction enzyme recognition site, with the exception of RM_61115 (SEQ ID NO: 179, RM_61117 (SEQ ID NO: 111), and RM_61118 (SEQ ID NO: 112).

1b) Improved mRNA and Base mRNA Constructs

A base mRNA, also referred to as a base construct (or starting construct) of a CAR (e.g., CAR1, CAR2, CAR25, CAR7, and CAR22) comprised the UTR pair UTR_1-UTR_11 (Tobacco etch virus 5′ UTR and Xenopus β globin, TEV-XBG), and a coding sequence of the CAR which was codon optimized using an algorithm from IDT (https://www.idtdna.com/pages/tools/codon-optimization-tool#get-started) (OPT6 for IDT method) or from GenScript (GenSmart) (OPT8). The tobacco etch virus 5′ UTR was previously shown to support enhanced translation activity in CHO cells in luciferase reporter mRNAs (https://pubmed.ncbi.nlm.nih.gov/8522182/). Xenopus β globin UTRs are also known to increase the translation efficiency of heterologous mRNA constructs in NIH 3T3 mouse cells (https://pubmed.ncbi.nlm.nih.gov/2762315/). The UTR pair UTR_1-UTR_11 (TEV-XBG) was used to demonstrate robust erythropoietin expression in mice (https://www.ncbi.nlm.nih.gov/pmc/articles/PMC3345990/). The widely used IDT codon optimization method was utilized here for the base constructs.

Two CAR1 base constructs were prepared: RM_61117 and RM_61362 (SEQ ID NO: 134), which had the same ORF (SEQ ID NO: 92) with the same codon-optimized sequence. Three CAR2 base constructs were prepared: RM_61118, RM_61512 (SEQ ID NO: 168), and RM_61363 (SEQ ID NO: 135) which comprised the same ORF (SEQ ID NO: 100) with the same codon-optimized sequence. The differences between RM_61117 and RM_61362 was that RM_61362 contained, double stop codons, a BsrGI recognition sequence between the 3′ UTR and the poly(A) tail, and a poly(A) of approximately 110 nt while RM_61117 contained a single stop codon and a poly(A) of approximately 90 nt. Similarly, RM_61118 contained a single stop codon and a poly(A) of approximately 90 nt while RM_61512 contained double stop codons, a BsrGI recognition sequence between the 3′ UTR and a poly(A) tail of approximately 110 nt. RM_61363 was similar in structure to RM_61512, except that RM_61363 contained triple stop codons instead of the double stop codons in RM_61512. A list of mRNAs encoding CAR1 and CAR2 that were screened and tested are disclosed in FIG. 3C.

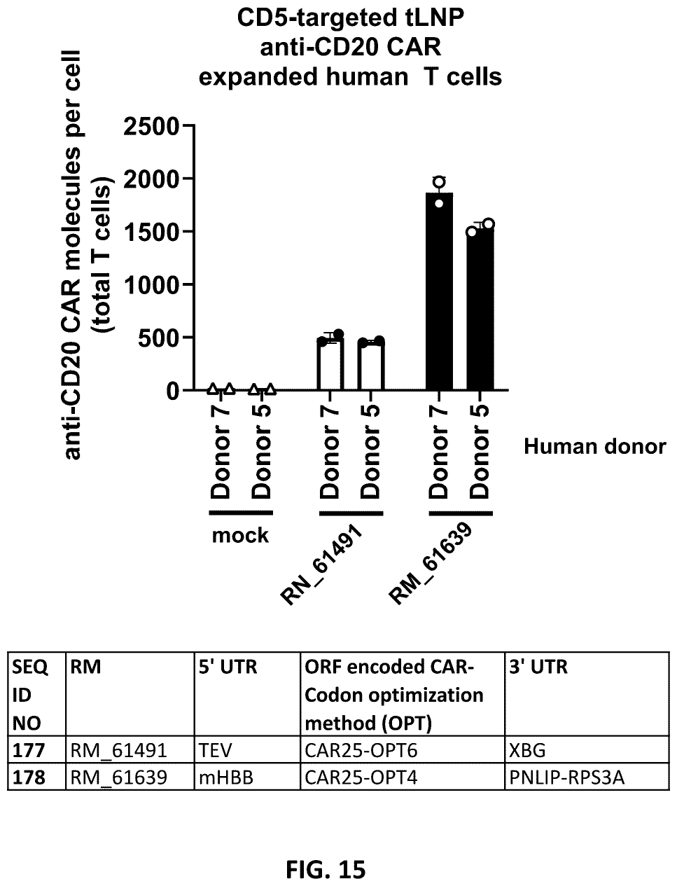

Base construct for CAR25 was RM_61491 (SEQ ID NO: 177) having ORF sequence encoding CAR25 which had been codon optimized by IDT (OPT6). A list of mRNAs encoding CAR25 that were tested are disclosed in table of FIG. 15.

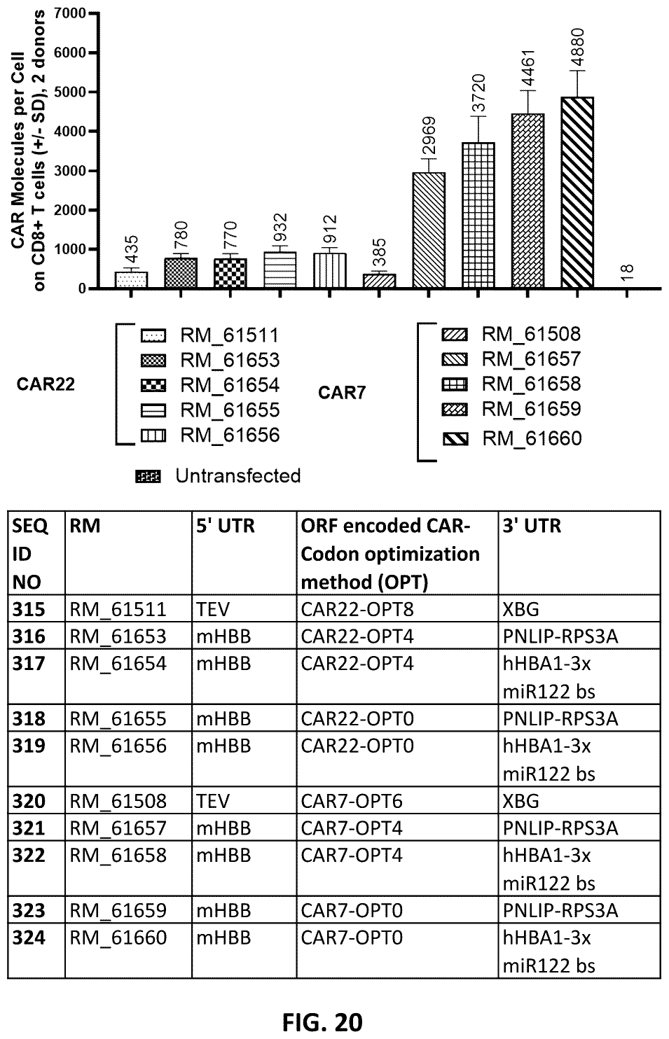

Base construct for CAR22 was RM_61511 (SEQ ID NO: 315) having ORF sequence encoding CAR22 which had been codon optimized by GenSmart method (CAR22-OPT8, SEQ ID NO: 83). Base construct for CAR7 was RM_61508 (SEQ ID NO: 320) having ORF sequence encoding CAR22 which had been codon optimized by IDT method (CAR7-OPT6, SEQ ID NO: 80). A list of mRNAs encoding CAR7 and CAR22 that were screened and tested are disclosed in table of FIG. 20.

1c) mRNA Synthesis

In particular, the mRNA sequences were synthesized by in vitro transcription (IVT) using T7 RNA polymerase with a full substitution of uridine with N1-methylpseudouridine (N1Mψ) along with Cap-AG reagent (^{m7}G(5′)ppp(5′)(2′OMeA)pG) to allow co-transcriptional capping with a Cap1 structure. The templates comprised DNA fragments corresponding to the 5′ UTR, ORF and 3′ UTR sequences inserted between the T7 RNA polymerase promoter and a poly(A) tail comprising approximately 90 to 110 adenine residues, within a plasmid vector carrying a kanamycin resistance gene. This insertion was achieved using the Golden Gate (Engler et al., 2008, PLOS ONE. 3 (11): e3647) or Gibson (Gibson et al., 2009, Nature Methods. 6 (5): 343-345) cloning method. EcoR1, BamH1, and BsrG1 restriction sites were used for the insertion, while other restriction sites can also be applicable. The accuracy of the plasmid sequences was verified through next-generation sequencing and/or Sanger sequencing techniques. Circular plasmids were linearized at a type II restriction enzyme site, located downstream of the poly(A) tail. The linearized plasmid served as template for IVT. Following transcription, DNase I was added to digest the DNA template. The mRNA was then purified using a two-step chromatography process using OligoDT affinity chemistry for bulk capture and ion-pair reverse phase chemistry for a residual impurities removal. The quality and purity of the mRNA were confirmed using a fragment analyzer, and levels of dsRNA contamination were monitored by immunoblotting, demonstrating detected levels below the established lower limit of quantification.

(2) tLNP Preparation

The tLNP used in the tLNP-based screening as described in Example 3 were either CD5-targeted LNP or CD8-targeted LNP, wherein the LNP composition was composition F5 or F9 as described below. Other tLNP having different binding moiety and/or LNP compositions (e.g., embodiments disclosed herein) can also be utilized for similar tLNP-based screenings. For the in vitro studies all CD5-targeted tLNP used the F5 LNP composition and all of the CD8-targeted tLNP used the F9 LNP composition. The in vivo studies used the F9 LNP composition for all tLNP.

Composition F5 was CICL1:DSPC:CHOL:DMG-PEG2000:DSPE-PEG2000-MAL [58:10:30.5:1.4:0.1] and composition F9 was CICL1:DSPC:CHOL:DSG-PEG2000:DSPE-PEG2000-MAL [58:10:30.5:1.4:0.1]. The terminal group of the non-functionalized PEG was methoxy throughout the Examples unless stated otherwise. The N/P ratio (the ratio of positively-chargeable lipid amine (N=nitrogen) groups to negatively-charged nucleic acid phosphate (P) groups) was 6. After initial LNP formation a SATA-modified anti-CD5 or anti-CD8 antibody was reacted with the maleimide moiety to provide the final tLNP.

For preparation of a tLNP loaded with a mRNA construct as described in Example 1, the mRNA construct was encapsulated in a LNP via a self-assembly process. In the self-assembly process, an aqueous solution (pH=3.5) of the mRNA was rapidly mixed with a solution of the lipids dissolved in ethanol, then followed by stepwise phosphate and Tris buffer dilution and tangential flow filtration (TFF) purification. LNPs were stored at 4° C. until conjugation. Next, the SATA-modified anti-CD5 mAb or anti-CD8 mAb was conjugated to the above LNP to generate tLNP. Purified antibody was coupled to LNP via N-succinimidyl S-acetylthioacetate (SATA)-maleimide conjugation chemistry. The antibody was modified with SATA (Sigma-Aldrich) to introduce sulfhydryl groups at accessible lysine residues allowing conjugation to maleimide. SATA was deprotected using 0.5 M hydroxylamine followed by removal of the unreacted components by G-25 Sephadex Quick Spin Protein columns (Roche Applied Science, Indianapolis, IN). The reactive sulfhydryl group on the antibody was then conjugated to maleimide moieties on the LNPs using thioether conjugation chemistry. Purification was performed using Sepharose CL-4B gel filtration columns (Sigma-Aldrich). tLNPs (LNPs conjugated with a targeting antibody) were frozen at −80° C.

Particular compositions for precursors to tLNPs and tLNPs are disclosed in U.S. Provisional Patent application No. 63/505,424 filed May 31, 2023, 63/510,061 filed Jun. 23, 2023, and 63/520,303 filed Aug. 17, 2023, as well as PCT Application No. PCT/US23/72426 filed Aug. 17, 2023, each of which is incorporated by reference in its entirety. LNP and tLNP compositions can include those of Table 15. In various embodiments, N/P can be from 3 to 9 or any integer-bound sub-range in that range or about any integer in that range.

### (3) T-Cell Preparation

Expanded T cells and freshly activated T cells were used in order to monitor mRNA translation activities in effector memory- or memory-like state and effector like state of T cells, respectively. To generate activated T cells, naïve T cells were exposed to CD3/CD28 stimulation and cytokines as shown in FIG. 3A. The freshly activated T cells were prepared by T cell isolation followed by activation with Dynabeads™ Human T-Activator CD3/CD28 for 3 days. The mRNA dose level of 0.6 μg was used to transfect 0.2 million of the cells in each well of a 96-well plate in triplicates. Activated T cells become expanded after incubation for approximately 10 to 14 days, after which time the overall cell number increases approximately 100-fold shown in FIG. 3A. The expanded T cells were prepared by first activating the isolated T cells with Dynabeads™ Human T-Expander CD3/CD28 for 5 days followed by approximately two weeks of expansion in OpTmizer™ CTS™ T cell Expansion medium supplemented with recombinant human IL2.

(4) In Vitro mRNA Screening

A tLNP-based mRNA screening, both in vitro and in vivo, employed a funnel model with selection criteria of mRNA constructs based on the CAR expression to determine optimal mRNA ORF, 5′ UTR, and 3′ UTR combinations. Screening rounds 1-3 (e.g., described in Example 1) utilized expanded human T cells from two different donors (Donors 1 and 2). In screening round 4 (e.g., described in Example 2, freshly activated T cells were used from two donors (Donors 3 and 4) separate from the donors in screening rounds 1-3. In screening round 5 (e.g., described in Example 3), utilizing freshly activated human T cells from Donor 3 both CAR expression CAR function was examined as killing of Nalm6 tumor cells in vitro. Screening round 6 (e.g., described in Example 5) entailed in vivo screening for Nalm6 tumor control in NSG-PBL mice engrafted with PBL from Donor 7. CD5-targeted LNPs were used for transfection in rounds 1-5, and CD8-targeted LNPs were used in round 6.

It was determined that a dosage of 0.6 μg was in the dynamic range of response through the first round of screening.

### (5) CAR Expression Measurement by Flow Cytometry

CAR expression was monitored by flow cytometry using phycoerythrin (PE)-conjugated antibody which detects the scFv portion of the linker region of the CAR1 and CAR2 CAR molecules. The result was expressed as CAR molecules per cell after interpolating the PE signal to a standard curve generated by using PE-beads.

### Example 1: CAR Expression on Expanded T Cells Via tLNP-Based mRNA Transfection In Vitro—Screening Rounds 1-3

The goal of the mRNA improvement effort is to achieve an elevated and extended expression of CAR molecules in transfected cells. Untranslated regions (UTRs) and codon usages have been shown to modulate mRNA stability and translation activity in the art. Various combinations of UTR and codon usage in mRNAs encoding CD19 CAR and CD20 CAR have been tested to achieve this goal.

Screening using tLNP-based in vitro transfection was used to evaluate 66 mRNA constructs incorporating seven codon variations and fifteen UTR pairs.

0.6 μg mRNA was used for screening in expanded T cells from human Donor 1 and Donor 2, respectively. The expanded T cells (0.2 million) were transfected with CD5-targeted LNPs and containing one of the 66 mRNA constructs shown in FIG. 3C (20 CAR1-encoding mRNA constructs and 46 CAR2-encoding mRNA constructs) at a 0.6 μg mRNA dose level. CAR expression was monitored at time points of 6, 24, 48 hours, which was determined for each test article by three methods: 1) area under the curve (AUC) of CAR expression, 2) average number of CAR molecules per T cell at 48 hours, and 3) peak CAR expression (molecules per cell).

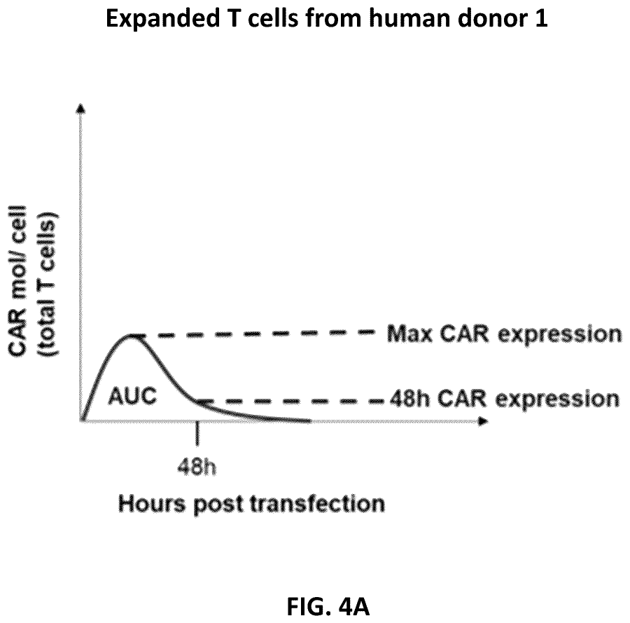

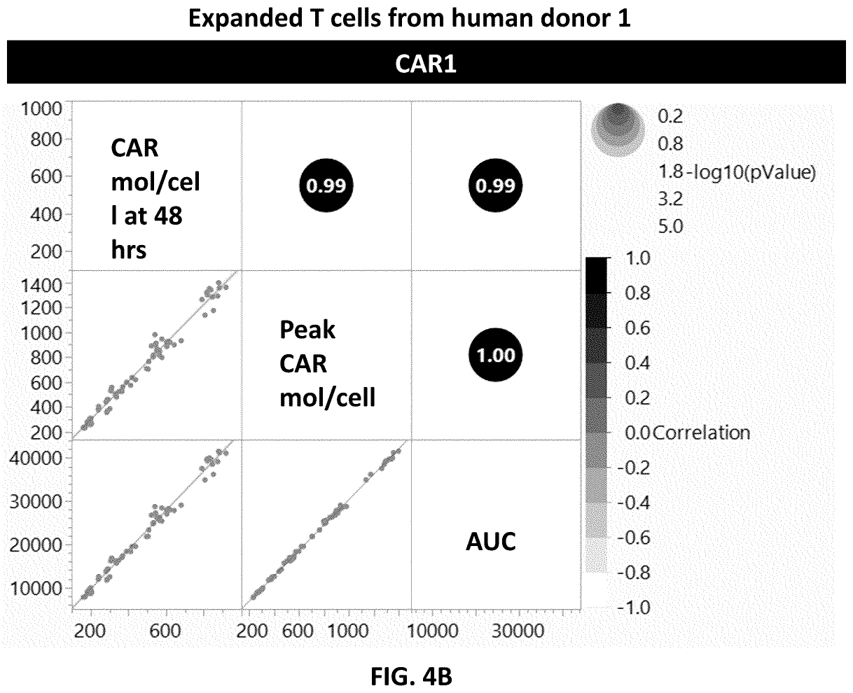

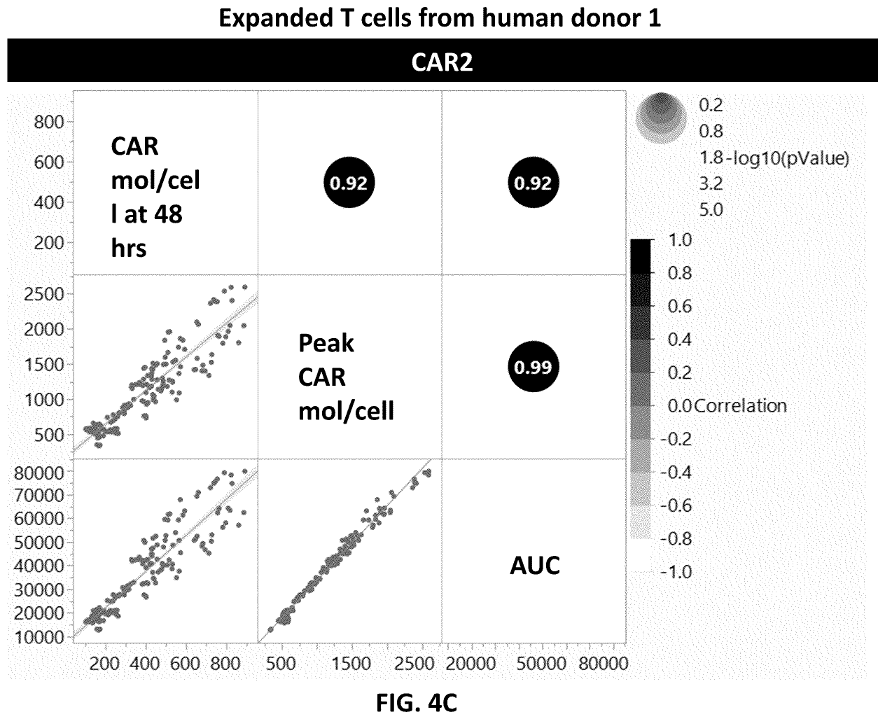

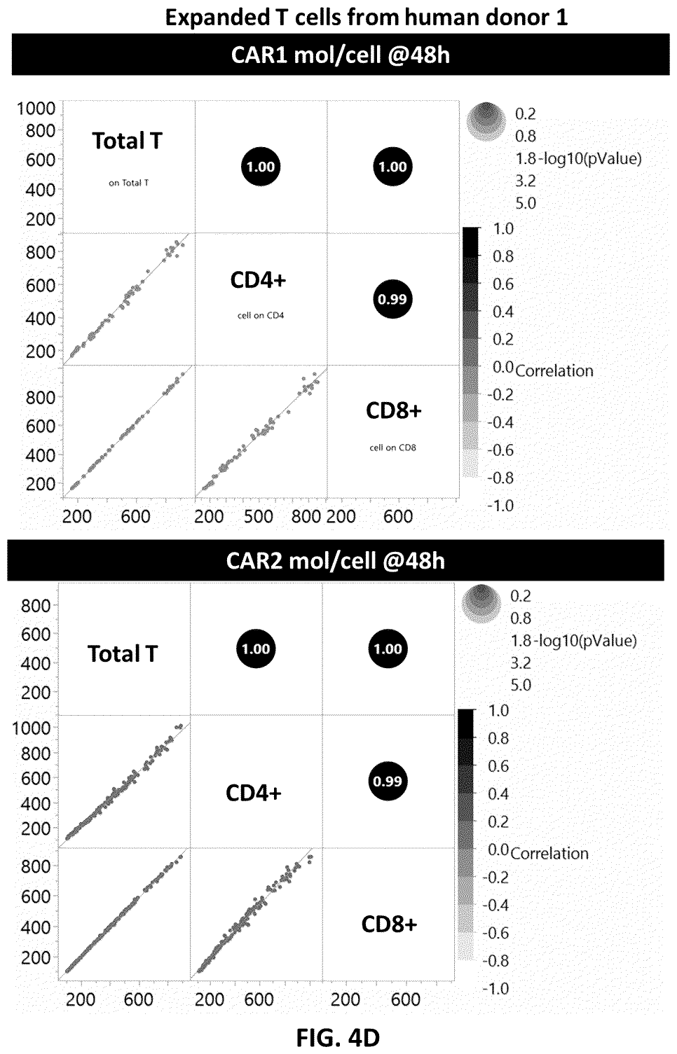

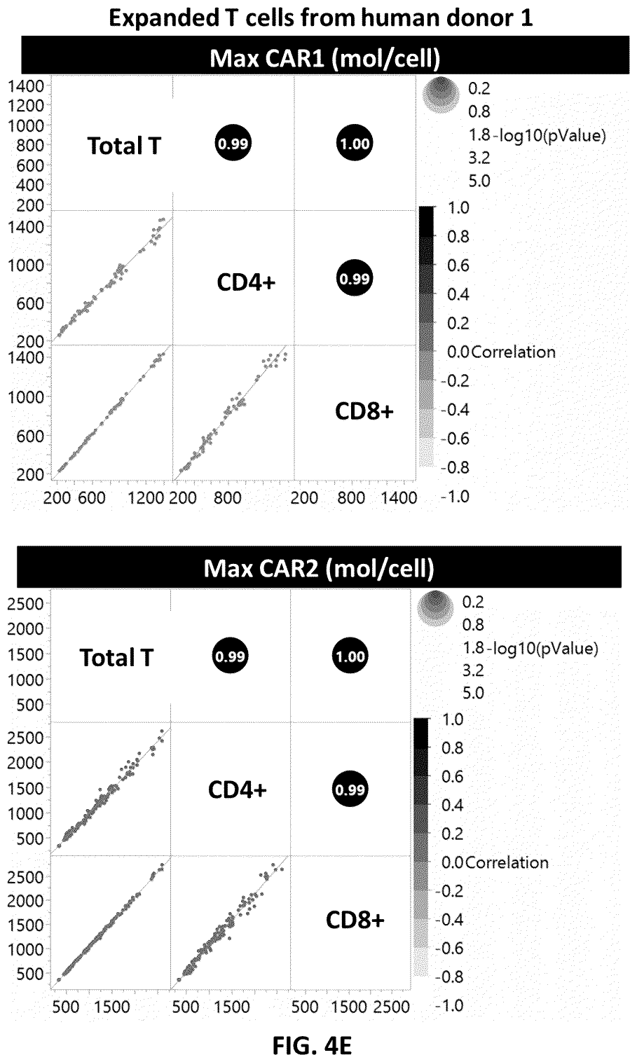

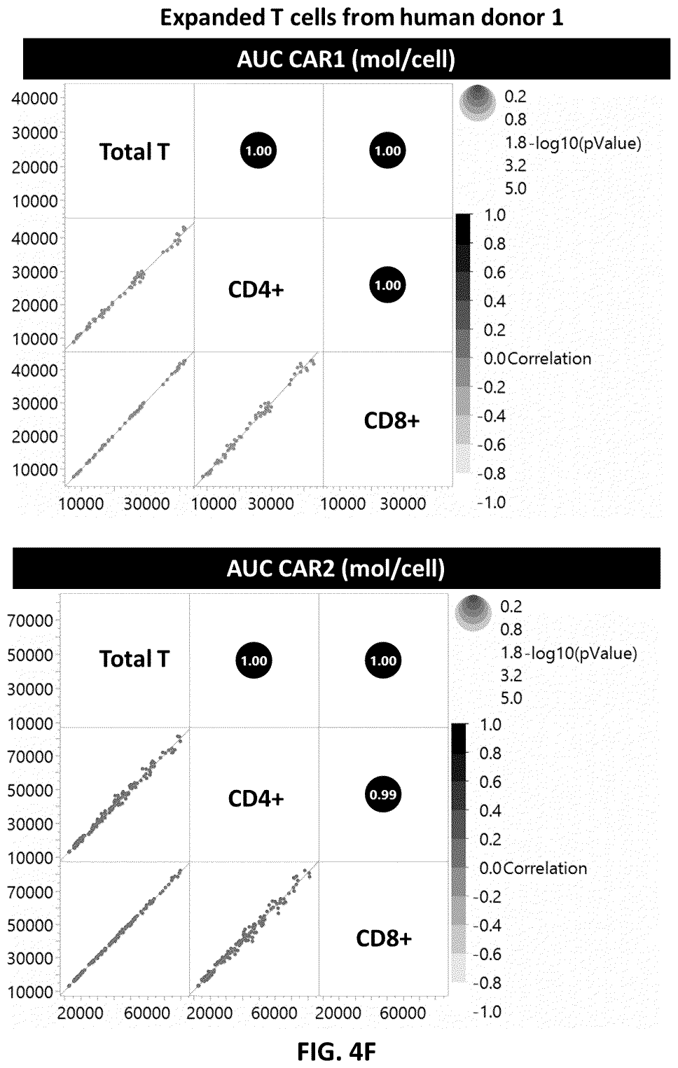

CAR expression was monitored at time points of 6-, 24-, and 48-hours post transfection, and the results of those experiments are shown in FIG. 3B through FIG. 4F. FIG. 3B shows time courses of CAR expression in expanded T cells for the 66 CAR encoding mRNA constructs summarized in FIG. 3C. The mRNAs that were selected for later screening in Example 5 were shown in solid black lines. The base constructs, RM_61117 and RM_61362 for CAR1, and RM_61118, RM_61363, and RM_61512 for CAR2, were shown in dotted black lines. Transfection of each test article was performed at least in triplicate.

The constructs were ranked in three tiers according to the mean CAR expression at 24 hours post transfection in triplicates: the first-tier level shows the constructs with 4-fold over the base constructs RM_61362 (for CAR1) or RM_61512 (for CAR2) on pan T cells at least from either donor, the second-tier constructs would show at least two-fold over the base constructs, and the third-tier constructs with CAR expression better than the base construct by less than two-fold better.

For CAR1 constructs, the first-tier constructs based on expression in T cells, as shown in FIG. 3B, and FIG. 3D through FIG. 3I were RM_61321, RM_61324, RM_61326, RM_61330, RM_61347, RM_61349, RM_61350, RM_61378, and RM_61379.

For CAR2 constructs, the first-tier constructs based on expression in T cells, as shown in FIG. 3B, and FIG. 3J through 3O were RM_61355, RM_61357, RM_61461, RM_61482, RM_61483, RM_61486, RM_61487, RM_61488, RM_61489, RM_61458, RM_61455, RM_61356, and RM_61358.

The CAR1 mRNA constructs, RM_61321, RM_61324, RM_61326, RM_61349 and RM_61350, and the CAR2 mRNA constructs RM_61357, RM_61461, RM_61486, RM_61487, RM_61488, and RM_61489 demonstrated 5-fold or more CAR expression at 24 hours post transfection than the base construct RM_61362 or RM_61512 in expanded T cells from at least one of the two donors. From these constructs, two codon usage strategies were utilized: OPT4 (maximized codon adaptation index for human, maxiCAI) and OPT1. As for 5′ UTR and 3′ UTR pairings used with these top performing CAR1 and CAR2 ORFs, the following UTR pairings were found in these mRNA constructs: UTR_5-12 (aptamer control-hHBA1), UTR_9-12 (eIF4G aptamer ×1-hHBA1), UTR_2-17 (mHBB-RPS3A), UTR_2-18 (mHBB-PNLIP_RPS3A), UTR_2-19 (mHBB-hHBA1 3× miR122 bs), UTR_2-20 (mHBB-PNLIP), UTR_2-21 (mHBB-RPS3A-PNLIP), UTR_6-15 (DEFA3-DEFA3), UTR_10-12 (hAlb_hHBA1), UTR_1-11 (TEV_XBG), and UTR_2-12 (mHBB-hHBA1) (the list of the UTRs and the combination codes can be found in the table of FIG. 8A).

The UTR pairs UTR_5-12, UTR_9-12, UTR_2-17, UTR_2-18, UTR_2-19, UTR_2-20, and UTR_2-21 were present in mRNA constructs showing 5-fold or more CAR expression than the base constructs RM_61362 or RM_61512 in expanded T cells from at least one of Donors 1 and 2.

The following CAR2 constructs demonstrated 4-fold greater expression over the base construct RM_61512 in expanded T cells from only one of the donors as shown in FIG. 3B, and FIG. 3J through FIG. 3O: RM_61458, RM_61455, RM_61356, and RM_61358.

All CAR1 constructs tested in expanded T cells led to CAR expression at 24 hours post transfection that was statistically higher than the base CAR1 constructs, RM_61117 and RM_61362. The CAR1 constructs in the second tier (constructs expressing CAR1 CAR at between two and four-fold more than the base construct, RM_61362, in expanded T cells) are RM_61348, RM_61329, RM_61340, RM_61341, RM_61351, RM_61353, and RM_61359, as shown in FIG. 3D through and FIG. 3I.

CAR1 constructs RM_61352 and RM_61334 showed less than two-fold increased CAR expression than the base construct RM_61362 in expanded T cells (i.e. in the third tier), yet still significantly higher CAR expression than the base constructs RM_61117 and RM_61362, as shown in FIG. 3D through FIG. 3I.

For CAR2 constructs in the second tier, the following constructs expressed CAR at between two and four-fold more than the base construct RM_61512 in expanded T cells from at least one of the two donors: RM_61459, RM_61484, RM_61485, RM_61454, RM_61451, RM_61453, RM_61515, RM_61460, RM_61456, RM_61411, RM_61468, RM_61360, RM_61466, RM_61467, RM_61465, RM_61452, and RM_61501, RM_61449, RM_61450, RM_61457, RM_61462, and RM_61463 as shown in FIG. 3J through FIG. 3O. The following CAR2 constructs led to CAR expression that was increased by <2-fold over the base construct, RM_61512, in expanded T cells of both donors (i.e. the third-tier): RM_61464, RM_61513, RM_61514, RM_61517, RM_61518, RM_61519, and RM_61520, as shown in FIG. 3B, and FIG. 3J through FIG. 3O. Of note, RM_61516 was the only mRNA construct that showed less CAR expression than the base construct, RM_61512, in expanded T cells. Six out of the above seven constructs contain a CAR2 ORF with OPT6 codon usage strategy, which was the same as the base constructs.

Expression was evaluated as CAR molecules per T cell at 48-hours post transfection, the peak or max CAR expression in CAR molecules per T cell, and the AUC of CAR expression These three criteria of CAR expression were highly correlated to each other as indicated by a Pearson correlation coefficient over 0.9 as shown in FIG. 4B and FIG. 4C. FIG. 4A through FIG. 4C show CAR expression from mRNA constructs from FIG. 3C via tLNP transfection of expanded T cells from human Donor 1 that were evaluated via three criteria. These criteria were visually exemplified in the FIG. 4A using a hypothetical CAR expression profile. The scatterplot comparison of the three evaluation methods on CAR expression shows CAR1 encoding mRNAs in FIG. 4B and CAR2 encoding mRNAs in FIG. 4C. A Pearson correlation is shown for each pair of criteria, and displayed proportionally with grey shading in a circle, the size of which corresponds to −log 10 of p-value. The lower the p-values, the higher the −log 10(p-values), therefore the bigger the circle. Gray shading in the circle is somewhat proportional to the absolute number of Pearson correlation. Just as in the scatter plot, the box in upper right corner shows a comparison (Pearson correlation with p-value info) between CAR mol/cell at 48 hrs and AUC. The one below is Pearson correlation between Peak CAR mol/cell, and AUC.

Peak CAR expression in both CAR1 and CAR2 mRNA constructs, the max number of CAR molecules per pan T cell, and AUC correlated better than either of those criteria with CAR molecules per cell at 48 hours. This is likely because peak CAR expression, which for most of the constructs occurs around the 24-hour time point as shown in FIG. 3B, has a greater impact on AUC than does 48-hour expression level which occurred well after the peak.

CAR expression in pan T cells was then analyzed against CAR expression in two different types of T cells: CD4⁺ and CD8⁺. FIG. 4D through FIG. 4E show scatterplot comparison of CAR expression in pan T, CD4⁺, and CD8⁺ T cells respectively, from CAR1 encoding mRNAs in the top panels and CAR2 encoding mRNAs in the bottom panels.

The expression of CAR in both CD4⁺ and CD8⁺ T cells were found to be highly correlated, with a Pearson correlation coefficient of over 0.99 as shown in FIG. 4D through FIG. 4E, demonstrating that the effect of UTRs and ORF improvements tested was similar in both subsets of T cells.

The CAR1 and CAR2 constructs shown in solid black lines in FIG. 3B, which were eventually selected for the later in vitro screening (Example 5) and a subset of the constructs were tested in NCG-Nalm6 mouse studies, consistently yielded high CAR expression levels at most of the time points. The CAR expression from the improved constructs at 24 hours post transfection was up to 5 to 6-fold higher than the base construct RM_61362 or RM_61512. This observation was consistent between the two doses and between CAR1 and CAR2.

One notable difference in the CAR expression profile between CAR1 and CAR2 constructs was that the CAR accumulation from CAR1 mRNAs was generally lower than CAR2 mRNAs at 6 hours, while the CAR1 CAR remained at higher levels at 48 hours than CAR2 CAR. This result suggests that the profile difference reflected the sequence difference in amino acids. On the other hand, the base CAR2 constructs, RM_61118 and RM_61512, shown in dotted black lines in FIG. 3B, exhibited peak expression at 6 hours, while expression from the top-performing constructs (solid black lines in FIG. 3B) peaked at 24 hours. This observation strongly suggested the extended functional half-life of the mRNAs can be attributed to nucleotide sequence improvement.

### Example 2: CAR Expression on Freshly Activated T Cells Via tLNP-Based mRNA Transfection In Vitro—Screening Round 4

For screening round 4, the 38 mRNA constructs (14 CAR1 and 24 CAR2 CAR mRNA constructs) were selected according to their CAR expression rank scores in CAR expression on expanded T cells as described in Example 1. CD5-targeted LNP containing the 38 mRNA constructs (the brief description of each construct is shown in FIG. 3C and the nucleotide sequences are shown in Table. 15) were transfected in freshly activated T cells (0.2 million) from two different donors, Donor 3 and Donor 4, at 0.6 μg mRNA dose level. CAR expression was monitored at time points of 24, 48, and 72 hours. The CAR molecules were detected using an anti-linker PE antibody.

The CAR1 and CAR2 mRNA constructs in the first tier of CAR expression on expanded T cells were selected to be tested in freshly activated T cells. In addition, constructs in the second tier from of CAR expression on expanded T cells were also selected according to the CAR expression ranking, which was derived from the mean score of the rank sum in each of three CAR expression criteria: 1) the AUC of CAR expression, 2) the average number of CAR molecules per T cell at 48 hours, and 3) the peak CAR expression measured in molecules per cell. RM_61359 and RM_61360 were selected, despite being mRNA constructs with a mediocre CAR expression, to determine if a correlation existed between the expanded T cells in screenings described in Example 1 and the freshly activated T cells in Example 2. The base constructs, RM_61117, RM_61362, RM_61118, and RM_61512 were utilized as reference constructs, which had low CAR expression in expanded T cells.

The set of mRNA constructs, 14 CAR1 encoding mRNA constructs and 24 CAR2 encoding mRNA constructs, were examined for CAR expression on freshly activated T cells obtained from two human donors, Donor 3 and Donor 4. As opposed to the expanded T cells, freshly activated T cells were expected to mimic effector T cells. For the activated T cell, the time point of 72 hours post tLNP transfection was chosen instead of 6 hours in the earlier screening rounds to identify mRNA constructs that exhibited extended CAR expression.

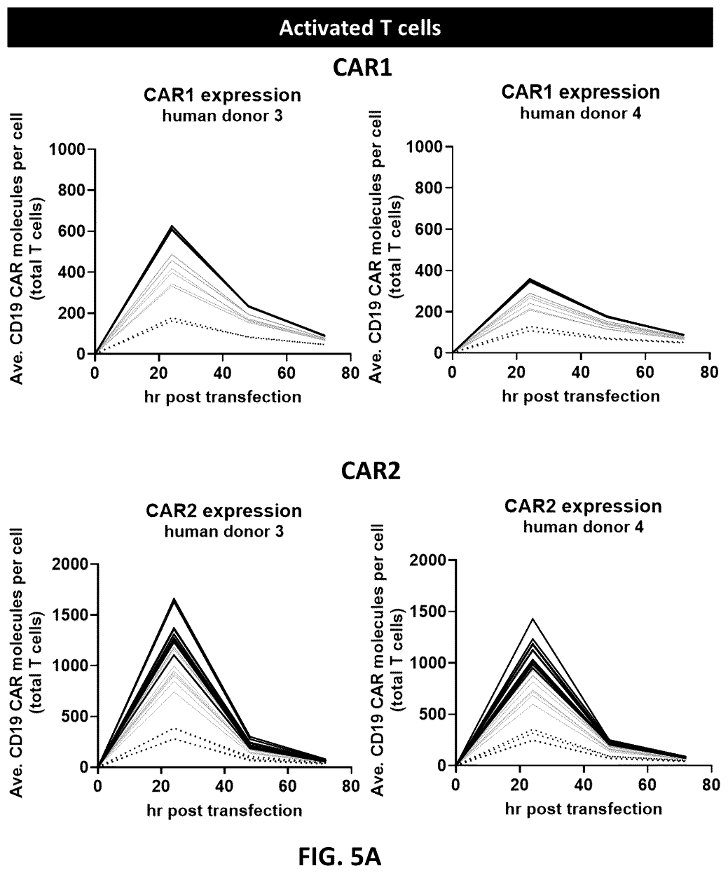

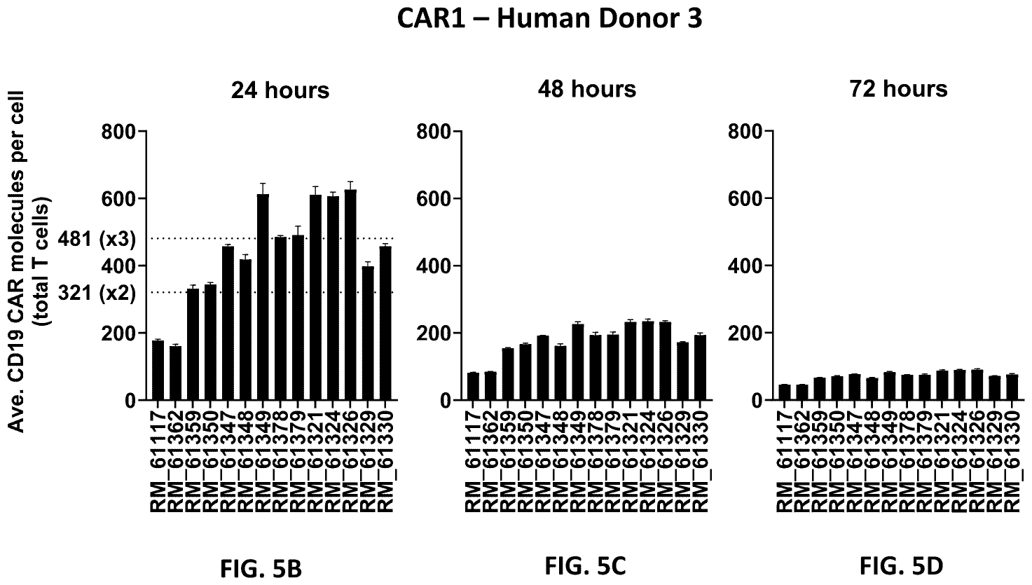

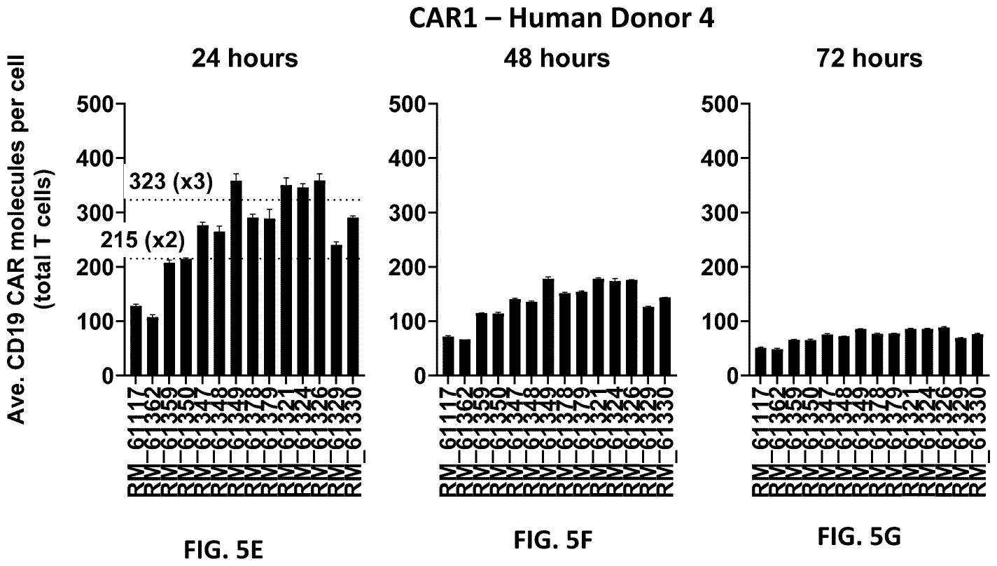

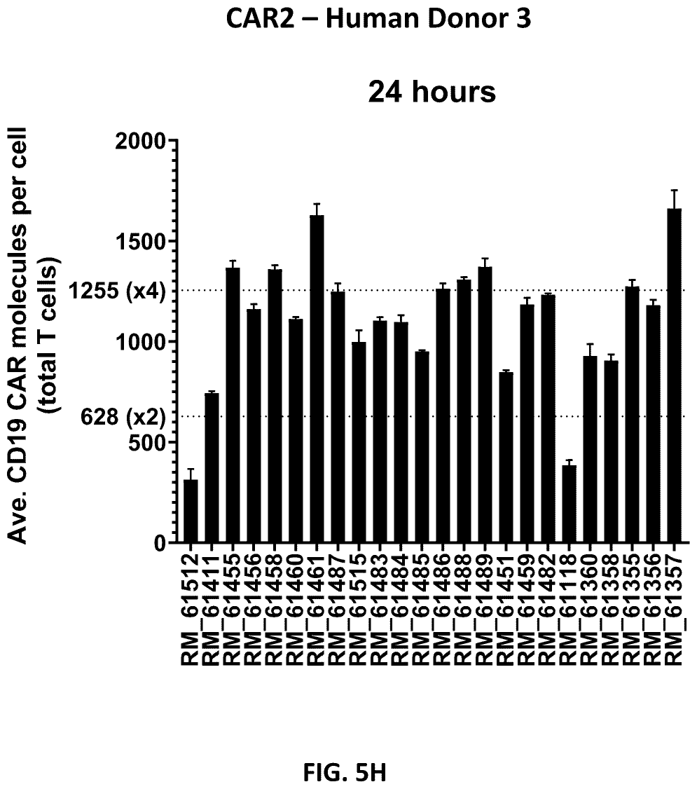

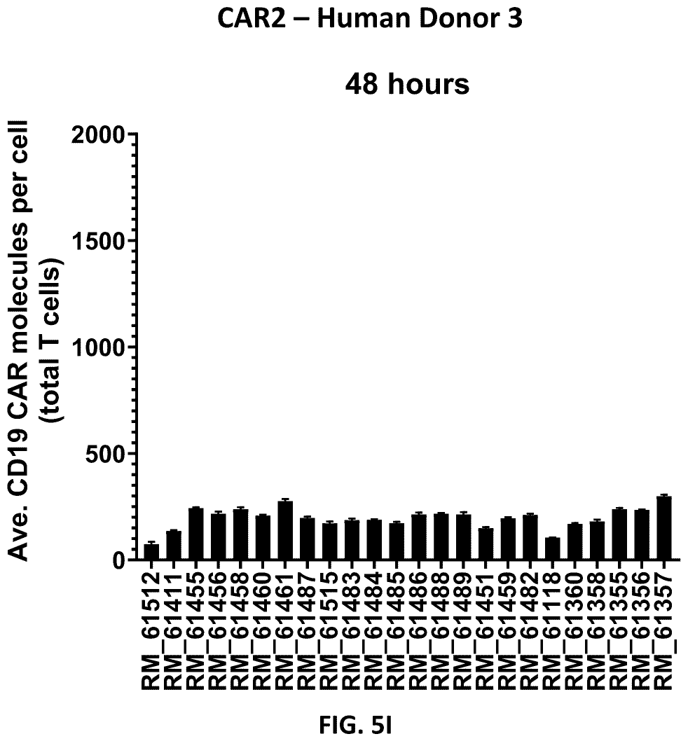

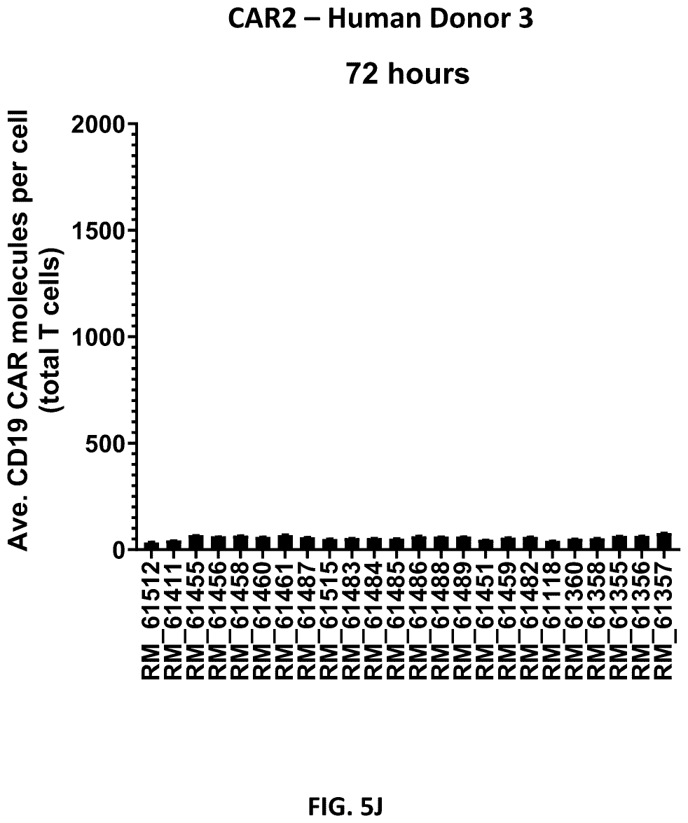

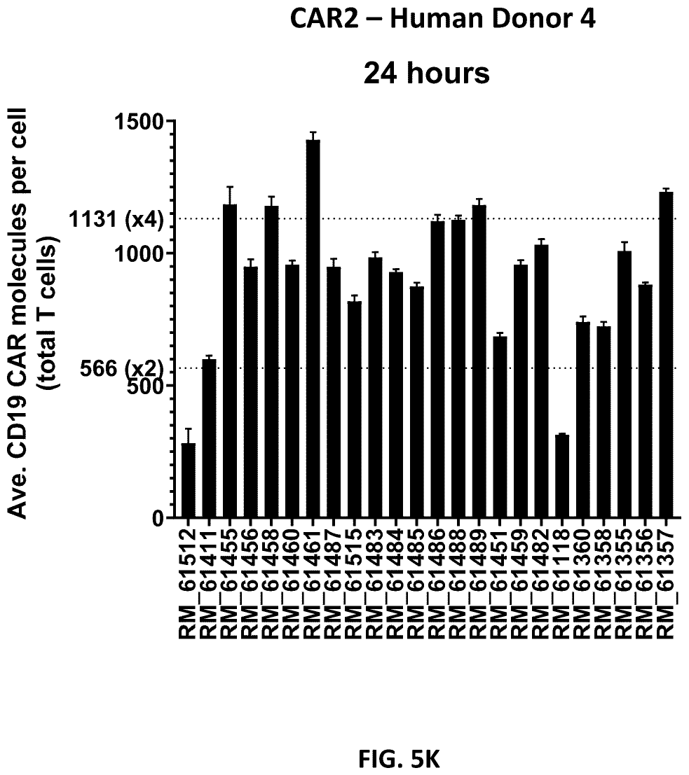

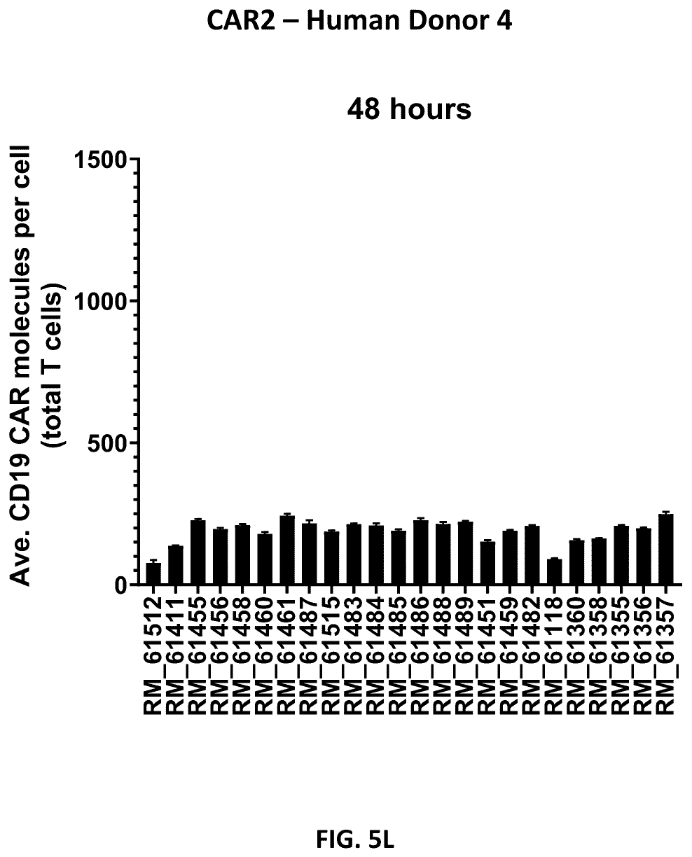

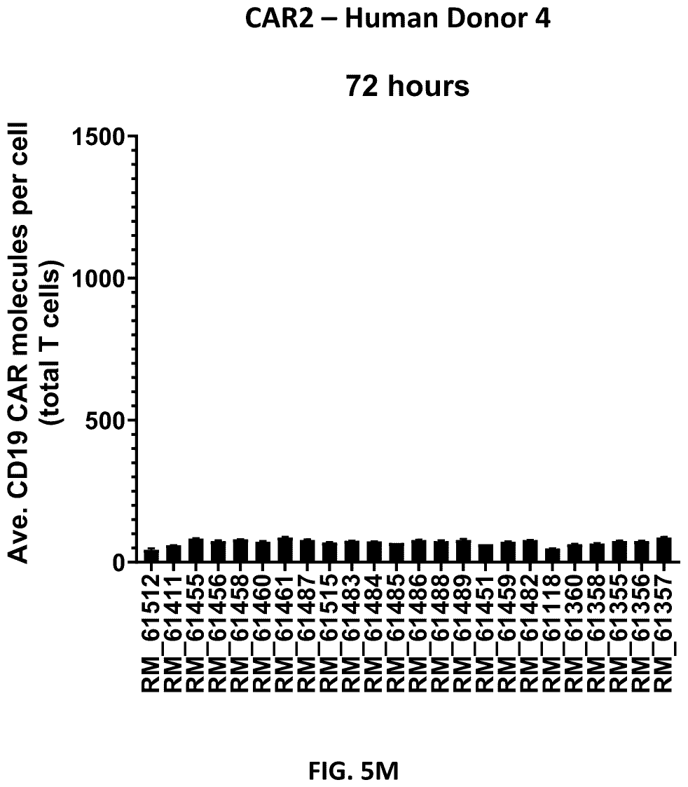

FIG. 5A through FIG. 5M show the expression of CAR 1 and CAR 2 over time in freshly activated T cells. FIG. 5A shows the time courses of CAR expression in freshly activated human T cells for mRNA constructs provided in FIG. 3C. The top-tier mRNA constructs that were used for later in vitro screening (Example 5) were shown in solid black lines. The base constructs, RM_61117, RM_61362, RM_61118, and RM_61512, were dotted black lines. Transfection of each test article was performed at least in triplicates. FIG. 5B through FIG. 5M show CAR expression for each CAR encoding mRNA construct at the 24-hour, 48-hour, and 72-hour time points. FIGS. 5B, 5E, 5H, and 5K contain horizontal lines that indicate either a two-fold, three-fold, or four-fold increase in CAR expression over the base constructs RM_61362, RM_61362, RM_61512, and RM_61512, respectively.

In freshly activated T cells, the CAR1 constructs were ranked in three tiers according to expression of CAR in average of triplicate transfection at 24 hours post transfection: the first-tier level showed the constructs with 3-fold increased CAR expression in at least one of the donor over the base construct RM_61362, the second-tier constructs showed at least two-fold increased CAR expression over the base construct RM_61362, and the third-tier constructs had increased CAR expression below the two-fold mark in both donors. The first-tier CAR1 constructs identified from activated T cells were, as shown in FIG. 5A through FIG. 5G: RM_61321, RM_61324, RM_61326, RM_61349, RM_61378, and RM_61379. The remainder of the CAR1 constructs tested in freshly activated T cells, RM_61330, RM_61347, RM_61348, RM_61329, RM_61350, and RM_61359 were second-tier constructs, showing at least a two-fold greater CAR expression than the base construct RM_61362 in T cells from at least one of the donors. None of these constructs fell in the third tier. RM_61321, RM_61324, RM_61326, RM_61349, RM_61378, and RM_61379 from the first tier in activated T cells overlapped with the first tier in expanded T cells.

For CAR2 constructs tested in freshly activated T cells, the first-tier constructs with about a 4-fold greater CAR expression over the base construct RM_61512 identified from T cells at least from one of the two donors are, as shown in FIG. 5A, and FIG. 5H through FIG. 5M: RM_61455, RM_61458, RM_61355 RM_61357, RM_61461, RM_61486, RM_61488, and RM_61489. The remainder of the CAR2 constructs tested in freshly activated T cells RM_61411, RM_61456, RM_61460, RM_61487, RM_61515, RM_61483, RM_61484, RM_61485, RM_61451, RM_61459, RM_61482, RM_61360, RM_61358, and RM_61356 were second-tier constructs, showing at least a two-fold greater CAR expression than the base construct RM_61512 in T cells from at least one of the donors as shown in FIG. 5H through FIG. 5M. None of these constructs fell in the third tier.

The CAR2 mRNA constructs from the first tier in activated T cells overlap with the first tier in expanded T cells.

On the other hand, RM_61356, RM_61358, RM_61487, RM_61482 and RM_61483 from the first-tier list of constructs in expanded T cells were in the second-tier list in freshly activated T cells.

The following top CAR1 mRNA constructs from both expanded and freshly activated T cells showed approximately a five-fold to six-fold increase in CAR expression over the base construct RM_61362 in expanded T cells from one of Donors 1 and 2: RM_61321, RM_61324, RM_61326, RM_61349, and RM_61350.

The top CAR2 mRNA constructs determined from both expanded and freshly activated T cells were: RM_61355, RM_61357, RM_61455, RM_61458, RM_61461, RM_61486, RM_61488, and RM_61489.

The CAR2 mRNA constructs that had approximately a 5-fold CAR expression increase over the base construct RM_61512 in expanded T cells at least from one of the donors and a greater than 4-fold increased CAR expression over the base construct RM_61512 in freshly activated T cells were: RM_61357, RM_61461, RM_61486, RM_61488, and RM_61489. On the other hand, RM_61487 showed −5-fold more CAR expression over the base construct on expanded T cells, yet failed to make the first tier in freshly activated T cells.

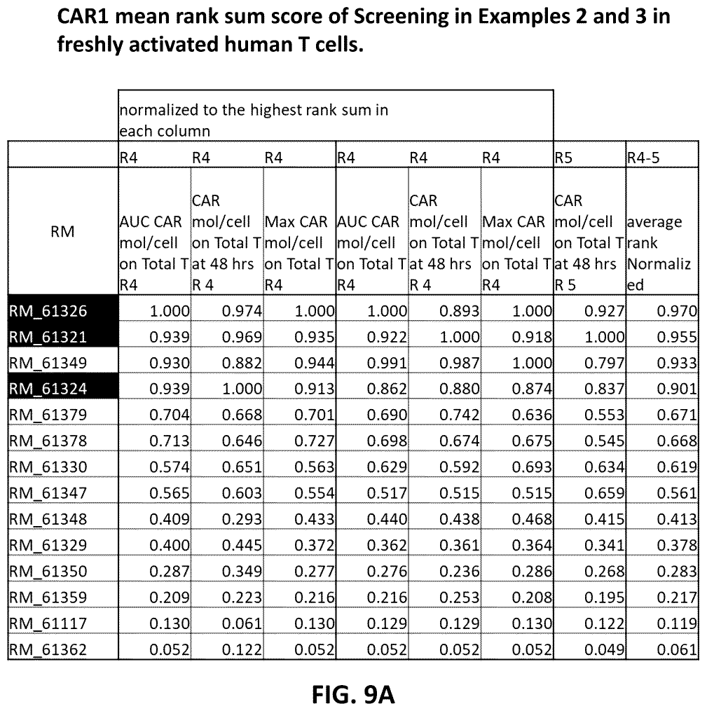

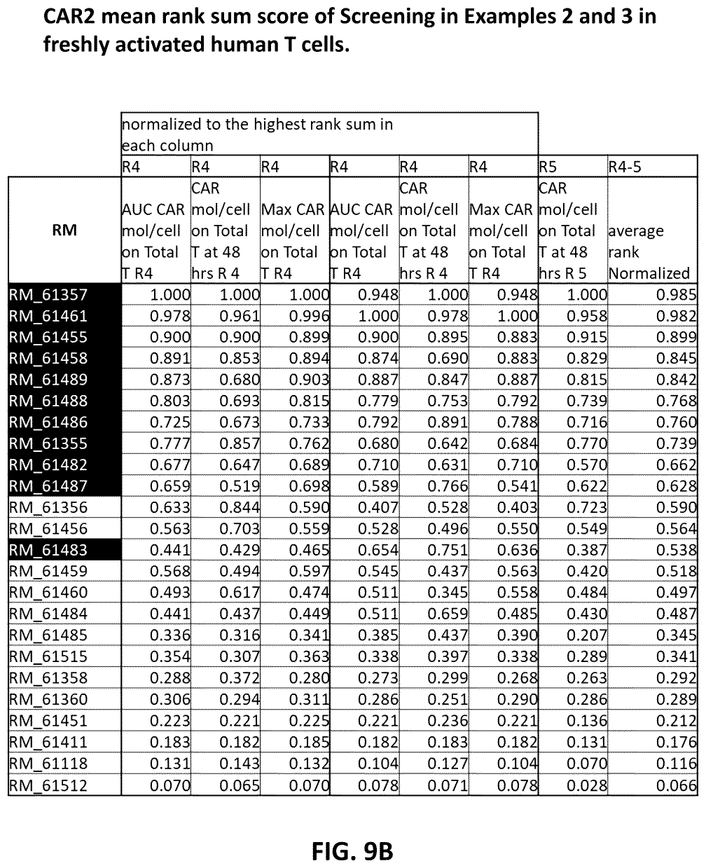

All of the top CAR1 and CAR2 mRNA constructs identified above except for RM_61349 were used for test article preparation for formulation of CD8-targeted tLNP to be further tested in vitro as in Example 5. Additionally, RM_61482 and RM_61483 were selected from the first-tier construct list from freshly activated T cells to be formulated with CD8-targeted LNP. Namely, the constructs formulated in CD8-targeted tLNP were RM_61326, RM_61321, RM_61324, RM_61461, RM_61489, RM_61357, RM_61486, RM_61488, RM_61355, RM_61455, RM_61458, RM_61482, RM_61487, and RM_61483. FIG. 9A and FIG. 9B summarize the rank order of the CAR1 and CAR2 mRNA constructs, respectively, based on the normalized mean of the rank sum scores calculated from the CAR expression with three criteria described in screening described in Example 1. RM_61461 and RM_61357 were top CAR2 mRNA constructs in freshly activated T cells from both donors, followed by RM_61455, RM_61458, and RM_61489 as seen in FIG. 5H through FIG. 5M. Four out of the above five constructs contain OPT4 ORFs. This reflects a codon-usage preference for OPT4 over OPT1 in activated T cells. Indeed, in pairs of CAR2 constructs using the same pair of 5′ and 3′ UTRs, RM_61461 vs. RM_61489, RM_61455 vs RM_61483, RM_61458 vs RM_61486, and RM_61355 vs RM_61482, RM_61456 vs RM_61484, and RM_61459 vs. RM_61487 the OPT4 constructs ranked higher than the OPT1 constructs for all but the last of these matched pairs. However, the advantages of the top CAR2 generating mRNA constructs diminished from 24 hours to 48 hours in the same way they did in the expanded T-cell in screening described in Example 1, shown in FIG. 3B, FIG. 3D through FIG. 3O. For example, the differences in CAR expression between RM_61461 and RM_61489 were smaller at 48 hours albeit still with a statistically significant advantage of RM_61461 in T cells of both Donors 3 and 4.

In both expanded and freshly activated T cells, the transfection of CAR1 mRNA constructs generated greater retention of CAR molecules in later time points than the CAR2 mRNA construct. However, at earlier time points, expression of CAR2 was higher. Elimination of target cells was observed as an early effect with in vivo generated CAR-T cells so that duration of expression was not an important parameter at least if there was robust expression early on.

It was also observed that the fold changes in CAR expression between the top-ranked constructs and the base constructs RM_61362 or RM_61512 were apparently lower in freshly activated T cells for both CAR1 and CAR2 mRNA constructs than in the expanded T cells. This trend held across different donors in expanded T cells and activated T cells.

### Example 3: In Vitro Determination of CAR Function Through Nalm6 Cell Death—Screening Round 5

The selected constructs based on the CAR expression on expanded T cells were subjected to validation for functional CAR expression in T cells co-cultured with Nalm6 cells, a human CD19⁺ tumor cell line, followed by monitoring the resultant cytotoxic effect on the cells.

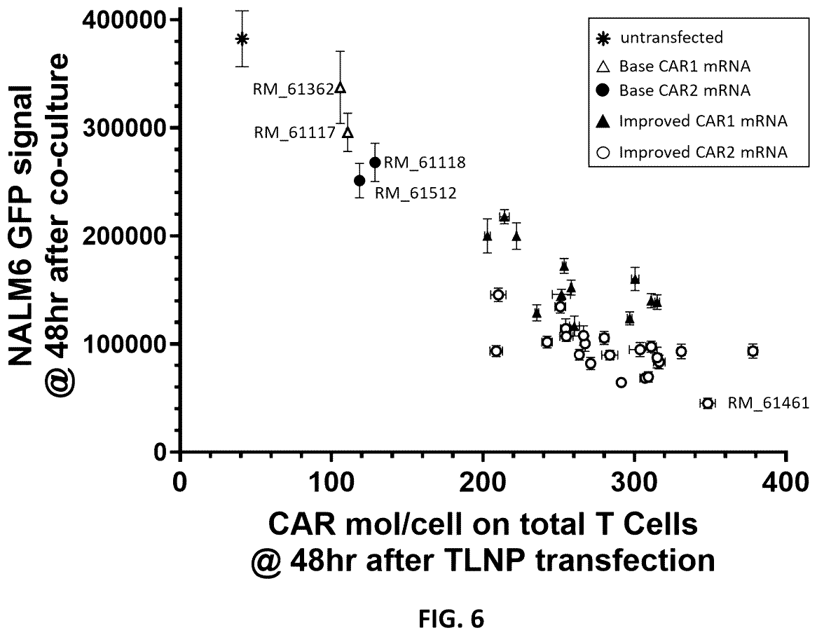

CD5-targeted tLNP containing one of the CAR mRNA constructs, as shown in FIG. 6 were used to transfect freshly activated T cells (0.2 million per well in 96-well plates) from Donor 3 at a dose of 0.6 μg in triplicates. At 48 hours post transfection, CAR expression was measured by flow cytometry. The CAR molecules were detected using an anti-Whitlow PE antibody. At 53 hours post-transfection, the T cells were pooled into one well, counted, and co-cultured with a fixed number of Nalm6 cells (10 thousand cells) at ratios of 6:1, 2:1, and 0.6:1. The Nalm6 cells were constitutively expressing green fluorescent protein (GFP). The GFP levels were monitored for the next 52 hours by Incucyte to determine the cytotoxic activity by the CAR-expressing T cells.

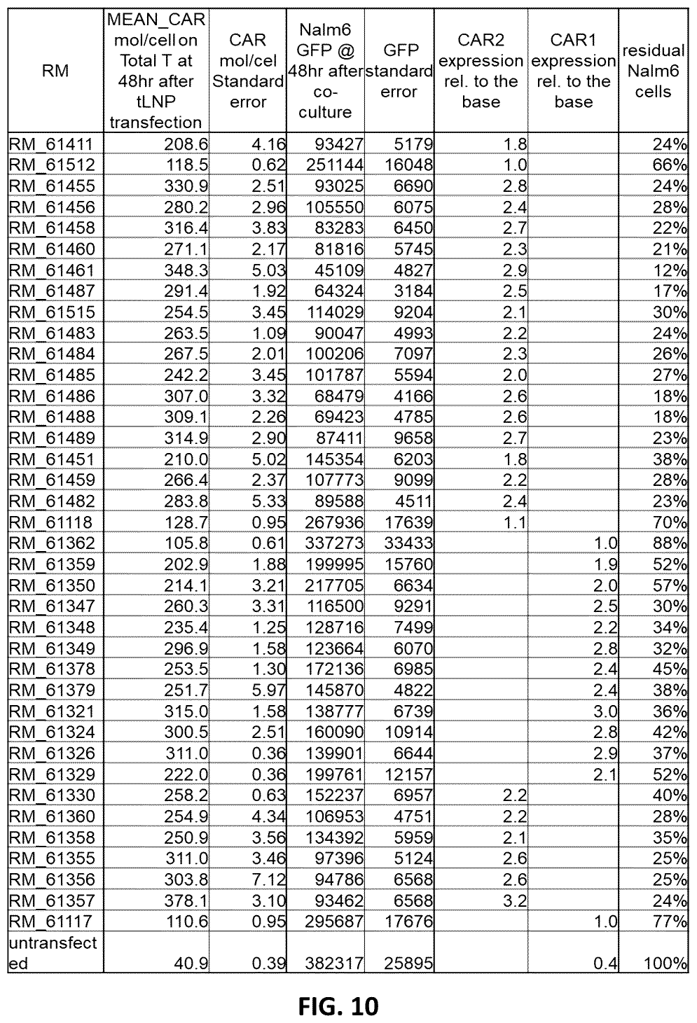

FIG. 6A shows a scatter plot of each construct for CAR expression in terms of the number of CAR molecules per cell and Nalm6 GFP signal at 6:1 ratio of T cells:Nalm6. The data points from base constructs are shown as open triangles for RM_61117 and RM_61362 or open circles for RM_61118 and RM_61512. Notably, the plot exhibits a correlation for CAR1 and CAR2 constructs and tumor cell killing, demonstrating that the increased CAR expression level from the mRNA engineering is accompanied with increased Nalm6 killing activities among the constructs evaluated in this experiment. Additionally, in this experiment, the CAR2 encoding mRNA constructs exhibited superiority over CAR1 encoding mRNA in cytotoxicity against the Nalm6 cells at similar levels of CAR expression on T cells. The most cytotoxic CAR encoding mRNA construct against the Nalm6 cells was RM_61461, which was amongst the first-tier list of constructs for CAR expression in the earlier screening rounds. FIG. 10 lists the data for each CAR encoding mRNA construct for CAR expression & Nalm6 GFP intensity used to generate FIG. 6. Besides RM_61461, RM_61487, RM_61486, and RM_61488 exhibited over 80% killing of Nalm6 cells as shown in FIG. 10.

### Example 4: Effect of UTR & Codon Usage Design Strategy on CAR Expression

The best performing CAR1 and CAR2 constructs in expanded and freshly activated T cells suggested common features of mRNA improvements including: OPT4 and OPT1 & OPT7 for codon optimization in common, as well as mHBB as a 5′ UTR in combination with tandem format (PNLIP-RPS3A/RPS3A-PNLIP) or individual 3′ UTR of PNLIP or RPS3A, and hHBA1-3× miR122 bs 3′ UTR.

A similar codon optimization preference was discovered when different codon optimization algorithms were tested with an identical 5′ and 3′ UTR pairs, UTR_1 and UTR_11 (TEV 5′ UTR and XBG 3′ UTR), shown in FIG. 7. Examining the AUC for CAR expression in molecules per pan T cell, the ORFs OPT4 and OPT1 sequences were the top codon usage strategies for CAR1 and CAR2 encoding mRNA constructs in both expanded and freshly activated T cells. The codon usage strategies OPT5, OPT2 and OPT3 were in the second tier in expanded T cell experiments. The least effective codon optimization was determined to be OPT6. T OPT6 was also found to have the lowest CAR expression in both expanded and freshly activated T cells. The OPT7 is slightly superior to OPT1, as seen in FIG. 7.

The overall data groupings shown in FIG. 8A were similar amongst OPT1, OPT2, OPT4, and OPT6. This finding suggests that the UTR sequence effect was constant and nearly independent of changes in ORF nucleotide sequences. The UTR_2-12 (mHBB-hHBA1), UTR_5-12 (aptamer control-hHBA1), UTR_9-12 (eIF4G aptamer ×1-hHBA1), UTR_10-12 (hAlb-hHBA1), UTR_6-15 (DEFA3-DEFA3), and UTR_2-18 (mHBB-PNLIP_RPS3A) 5′ and 3′ UTR pairs worked well to enhance overall CAR expression. Other UTR pairs showing improved performance included 3-12, 4-14 and 7-16. The 5′ and 3′ UTR pair UTR 2-19 (mHBB-hHBA1 3× miR122 bs) did well with both CAR1 and CAR2 OPT4I ORFs, but was not tested with other ORF sequences. Two additional UTR pairs, UTR_2-20 (mHBB-PNLIP) and UTR_2-21 (mHBB-RPS3A-PNLIP), were tested only in CAR1 OPT4. Both UTR combos paired with CAR1 OPT4 performed as well as UTR_2-17 (mHBB-RPS3A) in CAR1 OPT4. These three UTR pairings, along with UTR_2-19 (mHBB-hHBA1 3× miR122 bs), were the top UTR combos for the CAR1 OPT4 ORF. These UTR pairs were present in the CAR1 and CAR2 encoding mRNA constructs in the top tier.

Only the UTR pairing UTR_8-12 (CD8a-hHBA1) performed worse than the base UTR pairing UTR_1-11 (TEV-XBG), and this pattern was seen for with both CAR1 and CAR2 mRNA constructs irrespective of codon optimization. It was unexpected to find the CD8α 5′ UTR inefficient for mRNA translation in T cells, including CD8+ T cells.

Some exceptions to the UTR effect were noted. The least effective ORF in the overall ranking, OPT6, when paired with UTR_7-16 (CPA1_CPA1), was superior to other OPT6 mRNA constructs with other 5‘ and’3 UTR pairings. The 5′ and 3′ UTR pairing UTR_2-17 (mHBB-RPS3A) performed well, except when utilized with the OPT4 it was more beneficial for CAR1 than for CAR2.

The first-tier 5′ and 3′ UTR pairs for supporting mRNA translation in human T cells were, as shown in FIG. 3-AF, UTR_2-12 (mHBB-hHBA1), UTR_5-12 (aptamer control-hHBA1), UTR_9-12 (eIF4G aptamer ×1-hHBA1), UTR_6-15 (DEFA3-DEFA3), UTR_2-17 (mHBB-RPS3A), UTR_2-18 (mHBB-PNLIP_RPS3A), UTR_2-19 (mHBB-hHBA1 3× miR122 bs), UTR_10-12 (hAlb_hHBA1), UTR_2-20 (mHBB-PNLIP), and UTR_2-21 (mHBB-RPS3A-PNLIP).

A second group of 5′ and 3′ UTR pairs providing clear, if somewhat less improvement for supporting mRNA translation in human T cells included UTR_7-16 (CPA1_CPA1), UTR_3-12 (synthetic_hHBA1), and UTR_4-14 (HBA_AES-mtRNR1).

Performance of mRNA with the base UTR pairing UTR_1-11 (TEV-XBG) was generally improved when the other tested ORFs were substituted for the base IDT ORF.

### Example 5: CAR Expression Via Transfection of Human T Cells & PBMC with CD8-Targeted LNP In Vitro

The mRNA constructs (three CAR1 and 11 CAR2 constructs) selected through the screening in expanded and freshly activated T cells using CD5-targeted LNP were formulated instead with CD8-targeted LNP.

The mRNAs in CD8-targeted tLNP were transfected into expanded T cells, freshly activated T cells, and peripheral blood mononuclear cells (PBMC). The expanded T cells came from Donor 1, Donor 2, Donor 7, and Donor 5, the freshly activated T cells came from Donor 5 and Donor 6, and the PBMC came from Donor 8 and Donor 9. PMBC were obtained by apheresis, and then the T cells within were isolated and either activated or expanded as described in the earlier examples. The cells were loaded into 96-well plates, with each well containing 0.2 million cells each, except for the PBMC cell plates, which instead contained 0.4 million cells per well. CAR expression at 24 hours post transfection was detected by flow cytometry with a PE-human CD19 peptide, CAR molecules per CD8+ T cell were calculated from standard curve generated with PE-conjugated beads. CAR expression in the expanded T cells from Donor 1 and Donor 2 utilized a PE-conjugated anti-linker antibody instead.

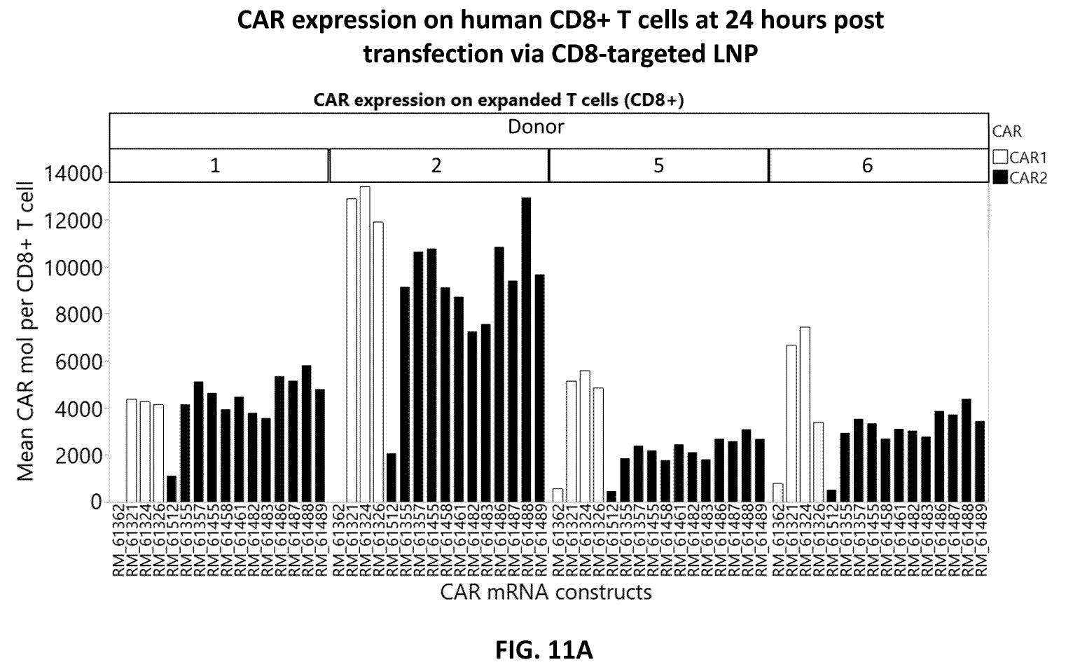

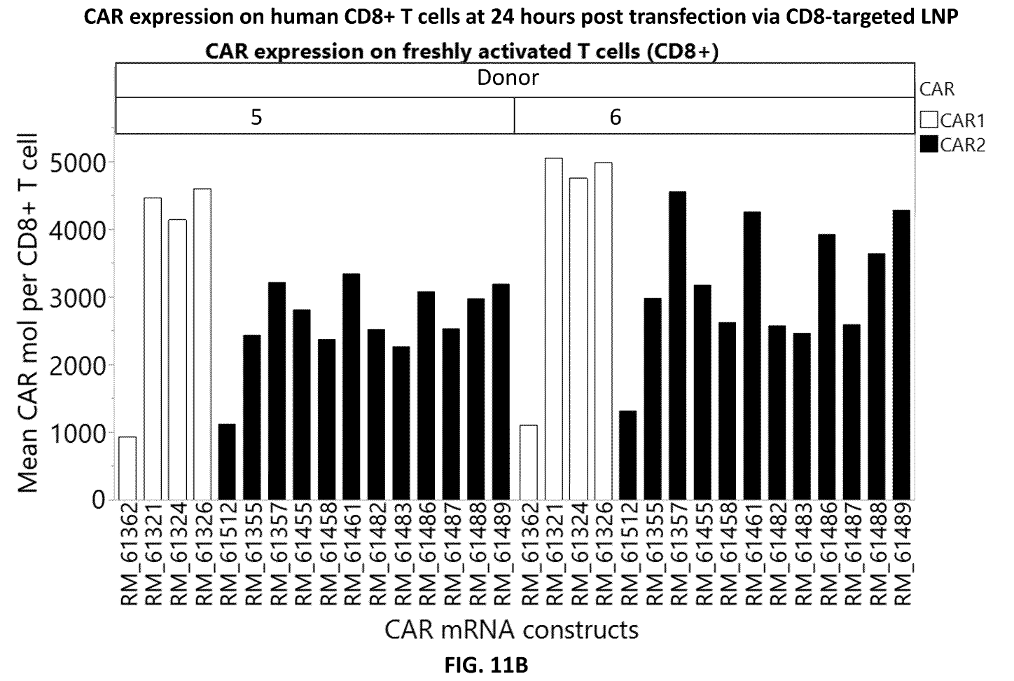

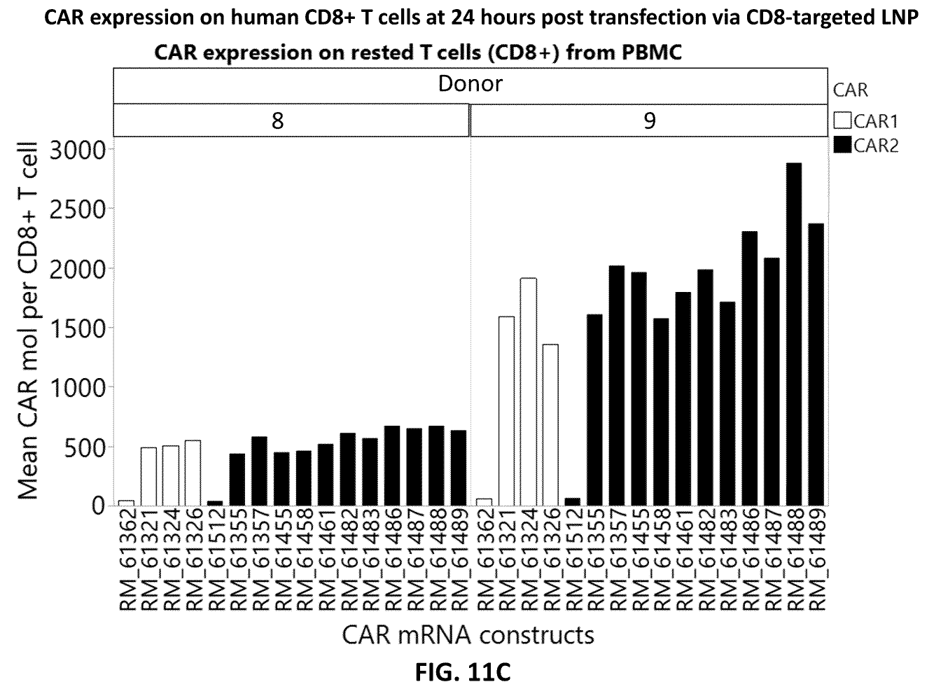

Consistent with results of CD5-targeted tLNP experiments in expanded and freshly activated T cells, all of the mRNA constructs tested with CD8-targeted tLNP demonstrated drastically higher CAR expression than the base constructs, RM_61362 (CAR1) and RM_61512 (CAR2) (FIG. 11). These results indicate the robustness of the mRNA engineering for CAR expression in the targeted T cell population whether the tLNP were targeted to pan T cells or CD8+ T cells. With respect to administration to a mammal, PBMC can be thought to best represent T cells in the circulating blood but freshly activated T cells can best represent T cells in secondary lymphoid organs which are efficiently transfected in vivo. Importantly, while the inherent efficiency of transfection and expression varies among PBMC, expanded T cells, and freshly activated T cells, these data indicate that the improved mRNAs each show similarly improved expression in each of these cell populations.

### Example 6: mRNA Improvement Screening for CAR1 and CAR2: In Vivo CAR Expression in NCG Mice—Screening 6

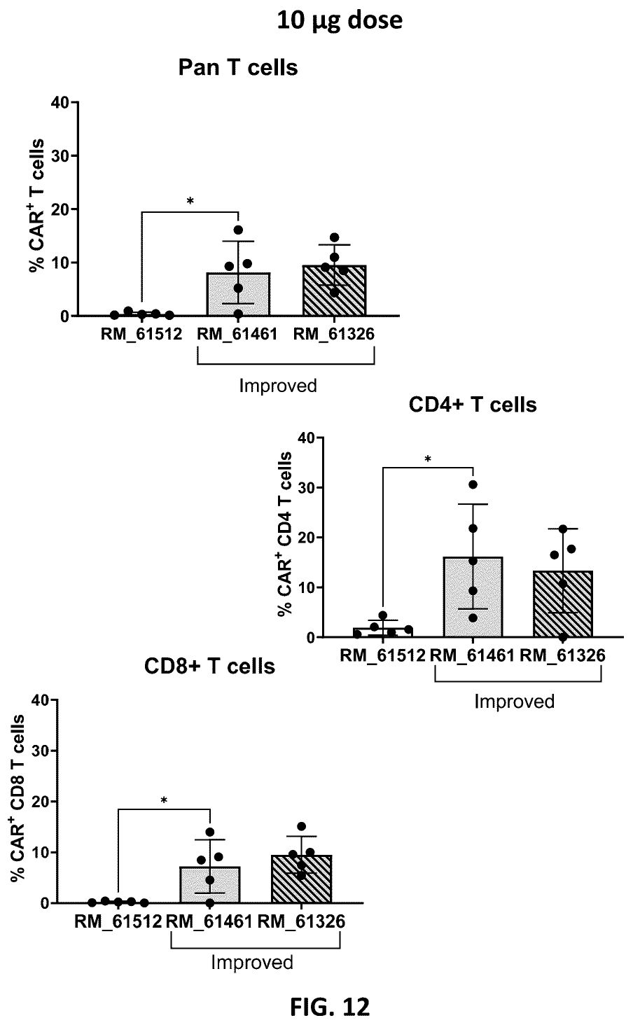

NCG mice (5-7 weeks) were purchased from Charles River, and 10 million human T cells from Donor 7 were injected intravenously. After 10 days of T cell engraftment in the NCG mice, Nalm6 tumor cells (0.5 million cells) constitutively expressing Firefly Luciferase (Luc) were injected intravenously into the mice. The mice were then evaluated for T cell engraftment, the frequency of human CD45+ cells in circulation, and tumor burden assessed by Luc signal and staged in groups with similar averages for both readouts. From the 18th day of the T cell engraftment, groups of 5 NCG mice were injected intravenously with human CD5-targeted tLNP containing either 10 μg of the base CAR2 mRNA RM_61512, the improved CAR2 mRNA RM_61461, or the improved CAR1 mRNA RM_61326, twice weekly for a total of 5 doses. Twenty-four hours after the third dose, each mice's blood was analyzed for CAR expression on T cells using PE-conjugated antibody against a scFv linker. The group of mice that were dosed with the improved CAR2 mRNA RM_61461 exhibited significantly higher populations of T cells that express CAR than those with the base CAR2 mRNA RM_61512. This trend held true in pan T cells, CD4+ T cells, and CD8+ T cells as shown in FIG. 12. This result indicates the in vitro versus in vivo correlation of the mRNA improvement applied on CAR2 encoding mRNA constructs.

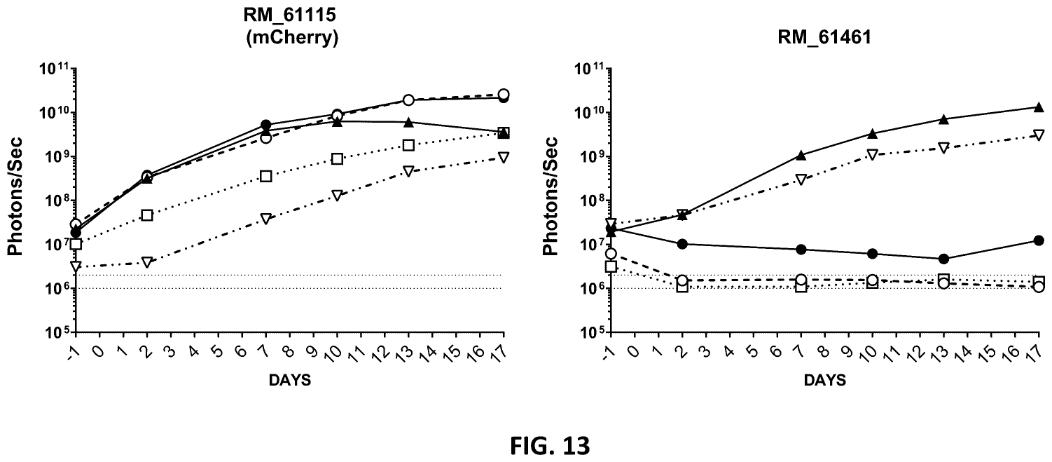

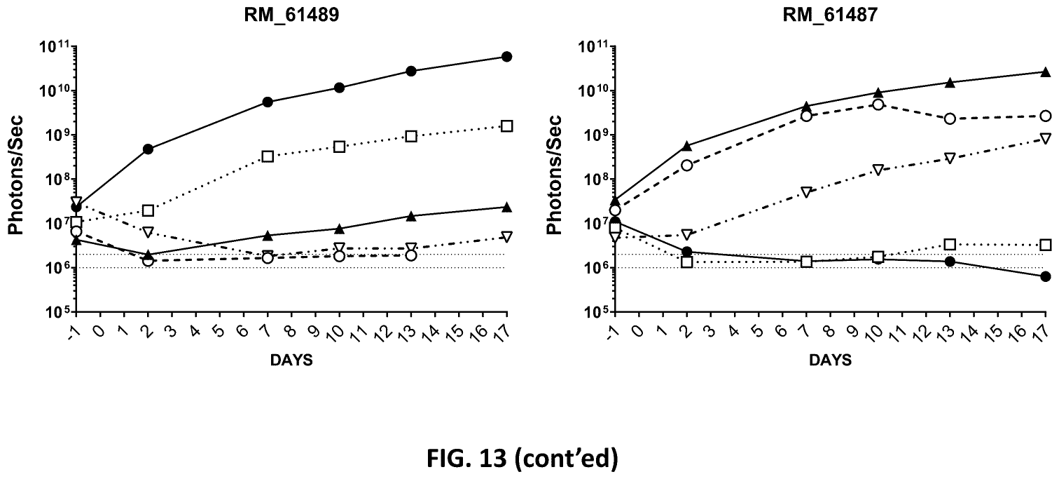

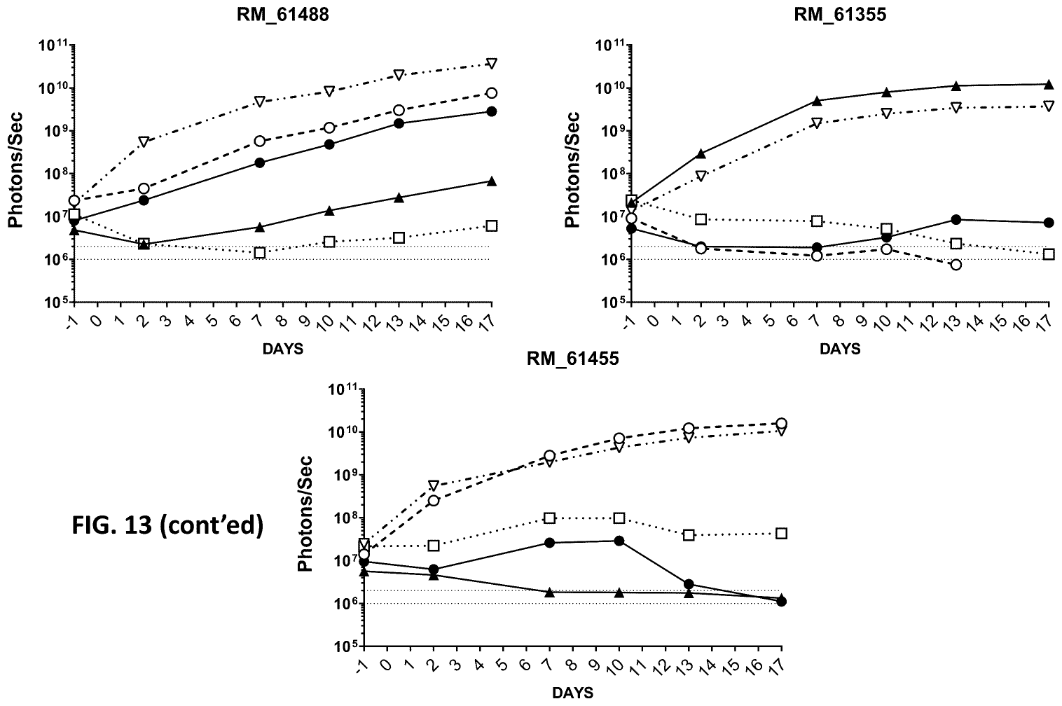

In another in vivo screening experiment, NCG mice (5-7 weeks) were purchased from Charles River, and 5 million human T cells from Donor 7 were injected intravenously into the mice. After 10 days of T cell engraftment, Nalm6 tumor cells (0.5 million cells) constitutively expressing Firefly Luciferase (Fluc) were injected intravenously into the mice. The mice were then evaluated for T cell engraftment, frequency of human CD45+ cells in circulation, and tumor burden assessed by Luc signal and staged in groups with similar averages of both readouts. From the 18th day of the T cell engraftment, groups of 5 NCG mice were injected intravenously twice weekly for a total of 5 doses of human CD8-targeted tLNP containing 10 μg of the following mRNAs: RM_61461, RM_61489, RM_61487, RM_61488, RM_61355, RM_61455, and mCherry (RM_61115) mRNA. Luciferin was injected on specified dates into mice intraperitoneally 6-10 min before in vivo imaging by IVIS, as seen in FIG. 13. In this experiment, the number of human T cells was reduced to half of the study shown in the other experiment described herein in Example 6 (10 million T cells from Donor 7) to reduce the frequency of human T cells at Day Zero. This was done to stress test the model, reduce risk of graft versus host disease (GvHD), and determine the differences of the effect between the test mRNA constructs.

All groups that were treated with tLNP containing CAR mRNAs demonstrated a reduction in Nalm6 tumor burden in two to three of the five mice per group as shown in FIG. 13. This demonstrated the efficacy of mRNA payloads in non-optimal conditions, where mice presented a lower T cell engraftment and higher initial Nalm6 tumor burden than in the previous study whose data are shown in FIG. 12. In contrast, each mouse in a control group injected with mCherry tLNP presented a robust growth of Nalm6 tumor continuously through the duration of the study. Most of the mice that responded to the CAR encoding mRNA treatment showed tumor reduction with the first tLNP treatment as shown in FIG. 13. A complicating factor in this study is that T cell engraftment varied from about 2 to 40% the day before the initial tLNP treatment dose, with most of the non-responders having less than 10% engraftment while most of the responder had greater levels of engraftment. There was also some variation in tumor burden at initiation of treatment. Accordingly, some combination of low T cell engraftment and higher tumor burden contributed to the observed treatment failures.

### Example 7: CAR Expression of Improved mRNA on T Cells in Blood in Non-Human Primates (NHP)

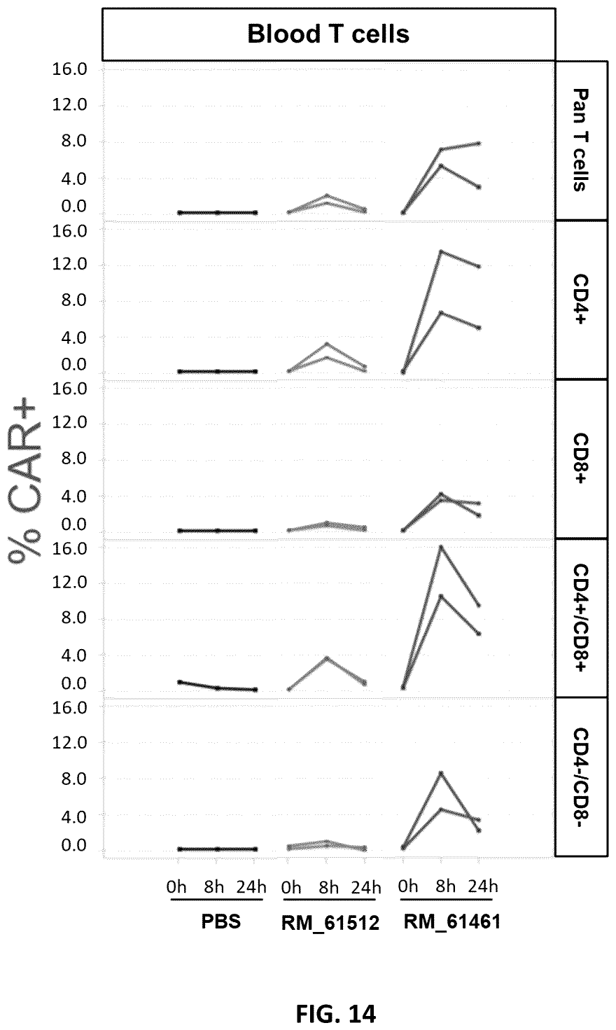

Non-human primates, Macaca fascicularis, were intravenously dosed with PBS (N=1) or CD5-targeted tLNP with composition F9 (Table 15) and a binder ratio of 0.6-0.7 at 2 mg/kg of base CAR2 mRNA (RM_61512) (N=2) or improved CAR2 mRNA (RM_61461) (N=2). Blood was collected from each NHP at 0 (pre-dose), 8 and 24 hours post dosing and analyzed for CAR expression using a PE-conjugated antibody against a scFv linker of the CAR molecule on pan T cells, CD4+, and CD8+ T cells as well as double positive and negative T cells. The group of NHPs that were dosed with RM_61461 exhibited 4 to 21-fold higher proportions of T cells expressing the CAR than those with base CAR2 mRNA (RM_61512) at 8 and 24 hours post dose (FIG. 14). Along with the similar experiments carried in NCG-PBL mice (FIG. 12 and FIG. 13, Example 6), this result indicates of the successful translation of the mRNA improvements applied to CAR2 mRNA from in vitro to in vivo results.

### Example 8: Improved mRNA Platforms Applied to Other CAR Constructs Such as Anti-CD20 CAR

To determine whether the UTR pairings and codon usage that led to enhanced expression of CAR1 and CAR2 can be applied to another CAR construct, an ORF encoding an anti-CD20 CAR, CAR25, was inserted into a base construct and an improved construct. CAR25 comprised the following domains in N- to C-terminal order: mouse Ig-kappa signal peptide, Leu16 anti-CD20 scFv, IgG4 hinge, CD28 transmembrane domain, 4-1BB co-stimulation, and CD3ζ signaling domain (stim) as shown in FIG. 1. A base CAR25 mRNA (RM_61491) and improved CAR25 mRNA (RM_61639) which contained one of the optimal ORFs, OPT4, and an optimal 5′ and 3′ UTR pairing, the 5′ UTR mHBB and the 3′ UTR PNLIP-RPS3A were prepared according to the procedures described in the Methods. This improved combination of UTRs and codon optimization was used in previous screening rounds in one of the best CAR2 mRNA, RM_61461.

Expanded human T cells (0.2 million) from two donors, Donor 7 and Donor 5, were transfected with 0.6 μg of the base CAR25 mRNA construct (RM_61491) and improved CAR25 mRNA (RM_61639). These mRNA constructs were formulated with CD5-targeted tLNP for transfection into expanded human T cells. The CAR expression at 24 hours post transfection from RM_61639 was 3-fold to 4-fold greater than the CAR expression from the base mRNA construct RM_61491 as shown in FIG. 15. This result confirms the applicability of the mRNA improvements to an mRNA encoding other CAR molecules.

### Example 9: In Vitro Assessment of CAR Expression, T Cells Activation, and B Cell Killing Using Improved mRNA

To assess the therapeutic potential of the transfected mRNA constructs encoding anti-CD19 CARs, T cells activation and B cell killing functions of the transfected cells were assessed.

CD8-targeted composition F9 tLNPs were formed encapsulating RM_61461 encoding anti-CD19 CAR2 (hereinafter tLNP-98219), RM_61639 encoding anti-CD20 CAR25 (hereinafter tLNP-982520), or mCherry mRNA. Human PBMC containing rested T and B cells from two healthy donors (A and B) were thawed and plated in U-bottom plates at 4×10⁵ cells per well. The tLNP were added to each well at dosages of 0.2, 0.6, and 2 μg per well, incubated for 1 hour, and then removed by a media wash. Using flow cytometry, CAR expression and T cell activation was assessed 24 hours later and B cell killing was assessed 72 hours later.

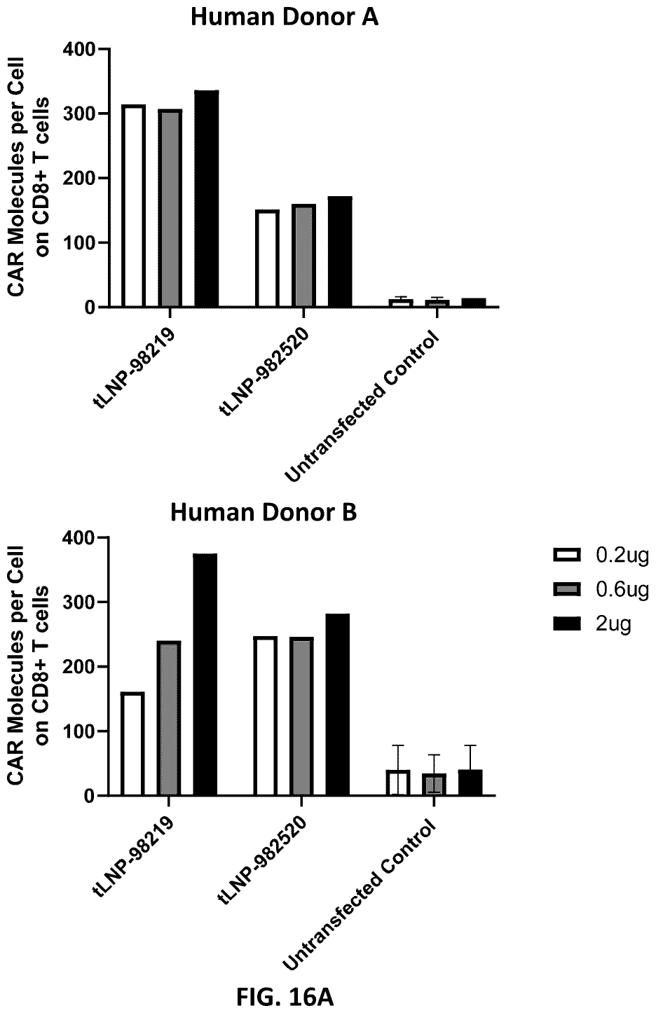

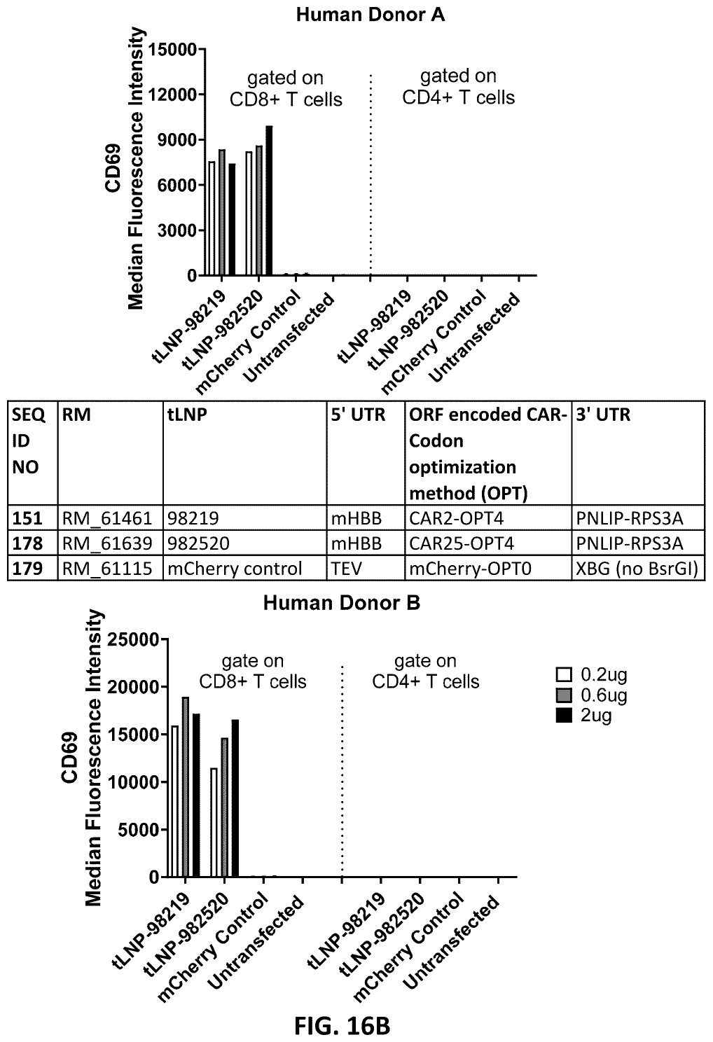

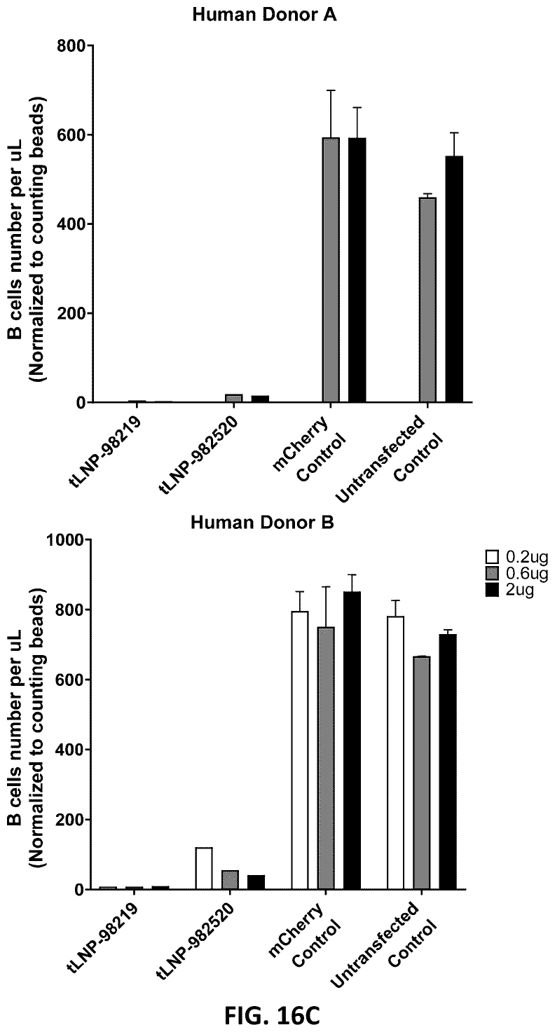

Expression of CAR2 was detected by staining with a CD19-PE conjugate and expression of CAR25 was detected by staining with an PE conjugated anti-G4S linker antibody. Both CARs were expressed at significant levels on CD8⁺ cells from both donors at between 100 and 400 molecules per cell (FIG. 16A). A consistent dose effect was not observed in the tested range. CAR expression was not observed on CD4⁺ T cells. The CAR expressing cells, that is the CD8⁺ but not the CD4⁺ T cells, also expressed the activation marker CD69, consistent with presence of B cells (FIG. 16B). Expression of either the anti-CD19 CAR or the anti-CD20 CAR led to profound and comparable killing of B cells (FIG. 16C).

### Example 10: In Vivo Assessment of Pharmacokinetic and Pharmacodynamic Effect of CARs Upon Transfection with tLNP-Encapsulated Encoding mRNA in the NSG-PBMC Model

Immunodeficient NSG mice (approximately 10 weeks old) were acclimated for at least 5 days after which 10 million human PBMCs were injected intravenously via the tail vein. After 17 days of engraftment mice were evaluated for frequency of human CD45+ cells in circulation and staged in groups with similar averages. Groups of mice were injected intravenously with a single dose (either 30 μg/animal or 10 μg/animal, roughly 1.5 or 0.5 mg/kg, respectively) of tLNP98219, or 30 μg/animal of a human CD8-targeted, F9 tLNPs encapsulating mCherry. Mice were sacrificed 24 hours after dosing and their spleens harvested to assess transfection and B cell depletion.

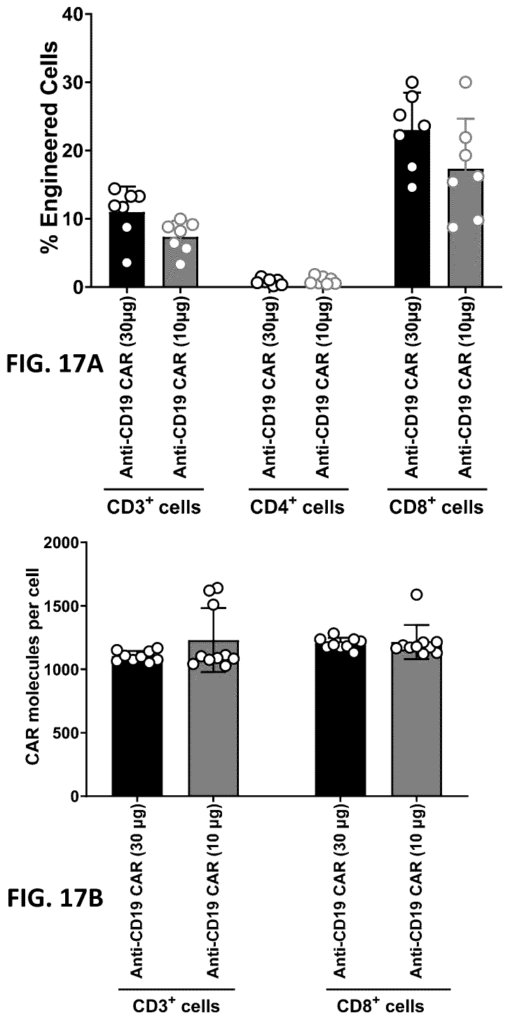

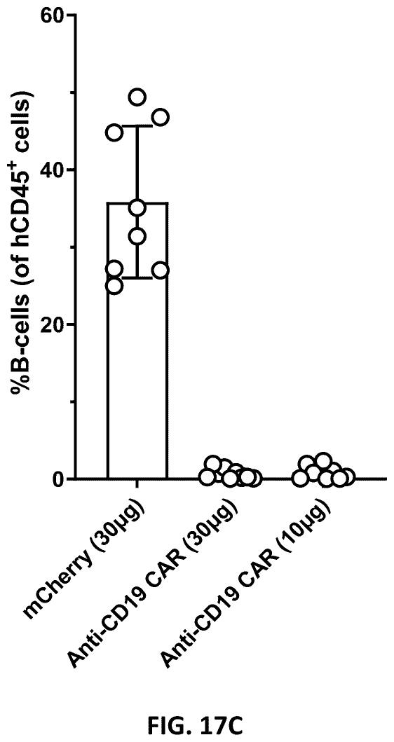

Efficiency of transfection was assessed in CD3⁺, CD4⁺, and CD8⁺ spleen cells and the level of CAR expression was assessed in CD3⁺ and CD8⁺ spleen cells. Means of about 7 and 11% of CD3⁺ cells and about 18.5 and 23% of CD8⁺ cells expressed the CAR for the 10 and 30 μg doses, respectively. CAR expression by CD4⁺ cells was minimal (FIG. 17A). The mean CAR expression levels in CD3⁺ and CD8⁺ cells were about 1100 to 1200 molecules per cell regardless of dosage (FIG. 17B). There was a deep depletion of splenic B cells to less than 2% of human CD45⁺ cells in the mice with CD8⁺ T cells transfected to express the anti-CD19 CAR with either dosage. In comparison, in the mice with CD8⁺ T cells transfected to express mCherry, B cells constituted about 25-50% of human CD45⁺ cells (FIG. 17C). Each of these effects was achieved within 24 hours of administering the tLNPs.

### Example 11: In Vivo Assessment of Pharmacokinetic and Pharmacodynamic Effect of CARs Upon Transfection with tLNP-Encapsulated Encoding mRNA in Non-Human Primate (NHP)

Non-human primates offered a more physiologically relevant model than immunodeficient mice transplanted with human PBMCs, enabling a fuller assessment of the effects of transfection with an anti-B cell agent. However, anti-human CD19 reagents such as CAR2 do not cross-react with NHPs such as cynomolgus monkeys. In contrast, anti-human CD20 reagents such as CAR25 do cross-react with cynomolgus monkeys. Accordingly, this study was designed to utilize both tLNPs carrying mRNA encoding CAR2 (RM_61461) and CAR25 (RM_61639). Transfection efficiency and expression level could be assessed for both CARs. The anti-CD20 CAR could be assessed for B cell depletion and, in addition to being a potential therapeutic agent in itself, served as a pharmacodynamic surrogate for the anti-CD19 CAR. Additionally, this study looked at the effect of targeting different T cell populations by utilizing tLNPs conjugated to anti-CD2, −CD5, and -CD8 antibodies. The tLNP delivering CAR2 and targeted to CD2⁺, CD5⁺, and CD8⁺ cells are referred to as tLNP-922219, -95219, and -98219, respectively. The tLNP delivering CAR25 and targeted to CD2⁺, CD5⁺, and CD8⁺ cells are referred to as tLNP-922520, -952520, and -982520, respectively.

The tLNPs were infused at a dosage of 2 mg/kg on days 1, 4, and 7 of the study and the animals were monitored until termination on day 15 (unless terminated earlier due to adverse events). Blood samples for complete blood count (CBC), peripheral blood flow cytometry (PBFC), and serum for biomarkers (cytokines and complement factors) were collected within 10 days before tLNP infusion for baseline. Blood samples were collected during the dosing phase of the study at 6, 24, 78, 96, 150, 168, 240 hours after infusion and at termination (generally 336 hours after infusion but in some cases earlier). Additional blood samples were collected at 208, 248, 264, and 279 hours for some animals. Serum samples were obtained at 4, 24, 76, 96, 148, 168, 240, 408 hours after infusion and at termination. Tissue and plasma samples were taken at necropsy upon termination.

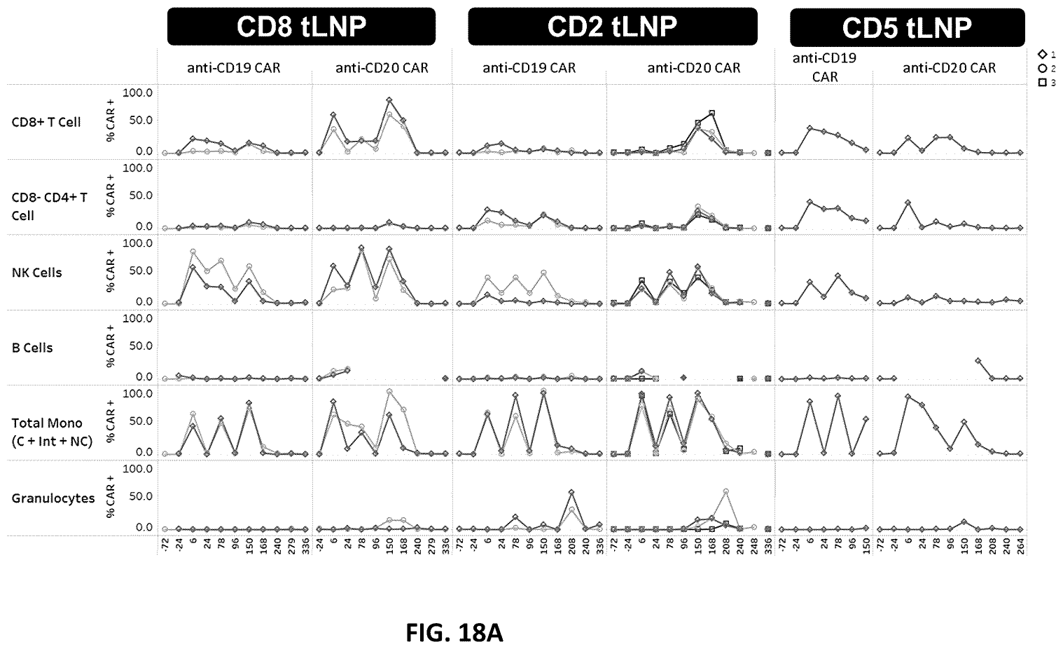

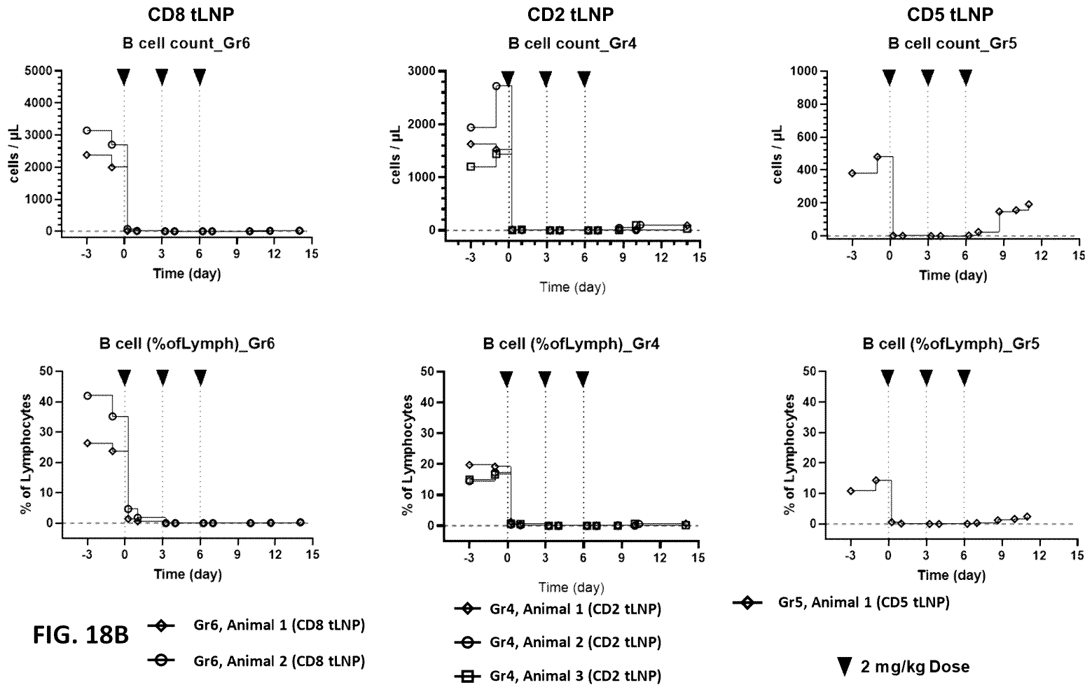

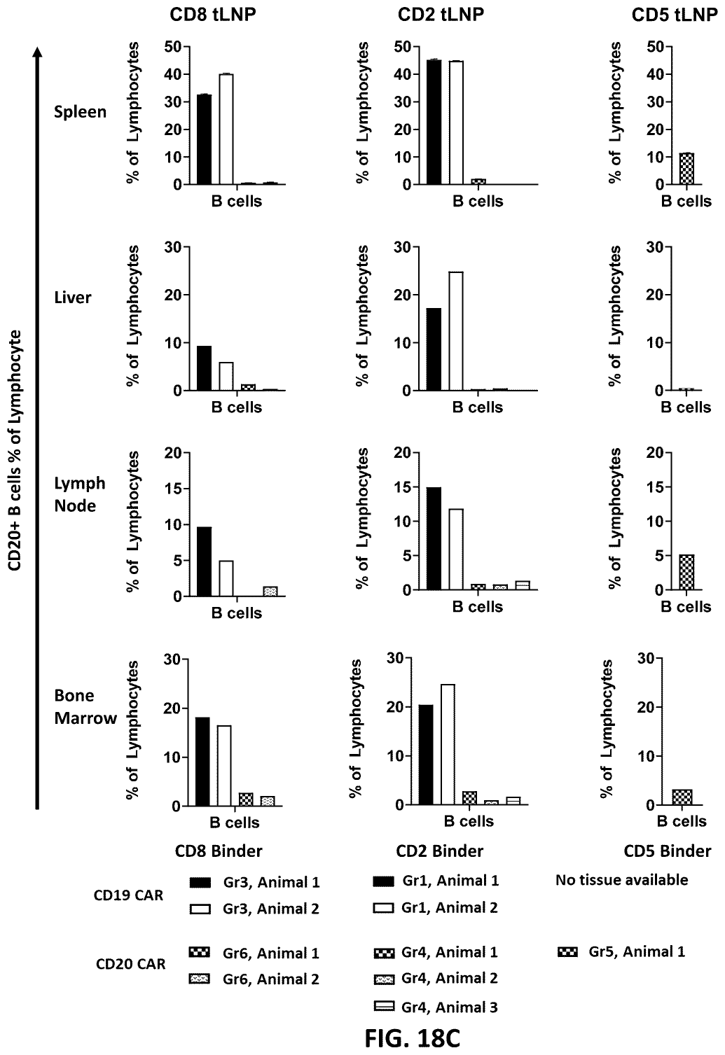

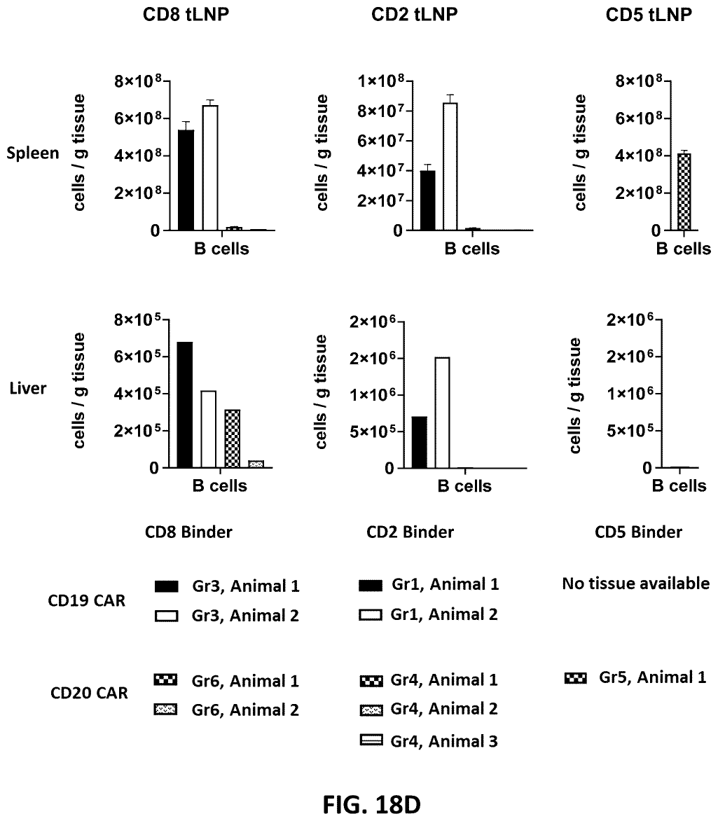

For both CARs, all three types of tLNP successfully transfected T cells. An overview of the data is presented in FIG. 18A. Flow cytometry of the blood samples showed that in general, a peak of expression was observed at the sampling 6 hours post dose administration and the CD8-targeted LNPs were highly selective for CD8⁺ cells with near baseline expression in CD4⁺ cells.

In blood, a deep depletion of B cells was observed as early as the first sample, at 6 hours after infusion of each of the tLNPs, and which lasted throughout the study (FIG. 18B). This contrasts with infusion of ex vivo generated CAR-T cells, which take several days to induce substantial depletion of B cells. A similar, profound and global B cell depletion was observed in the terminal tissue samples of spleen, liver, lymph node, and bone marrow for those animals that received the anti-CD20 CAR25 mRNA, but not those that received the anti-CD19 CAR2 mRNA, consistent with expectation given the lack of cross-reactivity of CAR2 to NHP (FIG. 18C and FIG. 18D).

This profound B cell depletion is particularly important since near complete or complete eradication of memory B cells would be needed to achieve a durable response in B cell-involved autoimmune disorders. This is not easily achieved with conventional B cell depleting agents (mAbs, relying on ADCC or complement activation), hence supporting a CAR T cell-based approach relying on the most potent immune effector cells (T cells). This ‘immune reset’ would involve ablation (depletion) of pathogenic cells, clearance of CAR molecules, and recovery of normal B cells without restoration of the autoreactive immune repertoire providing a durable response and normal immune competence. Due to the transient nature of CAR-T cells generated with mRNA instead of integrated DNA a long-term B cell deficiency such as that seen in some patients receiving traditional CAR-T cells would not be induced.

### Example 12: Expression of Functional Levels of Cas9

To demonstrate the generality of improving mRNAs as described herein, various ORFs encoding a different sequence outside of CARs, namely Streptococcus pyogenes Cas9 (SpCas9—SEQ ID NO: 84) were tested. The best performing codon optimization method OPT4 and best performing 5′ UTR and 3′ UTR pair: mHBB/PNLIP-RPS3A were selected. Additionally, OPT2 and the mHBB/hHBA1 UTR pair were also tested. As Cas9 is a bacterial protein strategies such as OPT1 and OPT7 that rely heavily on native sequence were inappropriate. After OPT4, OPT2 had performed best for contributing to improved expression of the anti-CD19 CARs reported in Examples above, as had the mHBB/hHBA1 UTR pairing. FIG. 19A, lower panel, summarizes the mRNA constructs encoding spCas9 that were tested. These mRNAs were transfected into both T cells and HSPC along with guide RNA and the level of gene knock-out assessed.

A coding sequence for SpCas9 that was codon optimized with the GeneSmart™ tool (Cas9-OPT8; SEQ ID NO: 71) and flanked by the TEV 5′ UTR and XBG 3′ UTR was used as the base construct, RM_61101 (SEQ ID NO: 306). The base construct included an N-terminal tag not included in the other constructs. Cas9′-OPT8 (SEQ ID NO: 86; having two silent point mutations to remove BspQI restriction sites) was also placed between the UTR pairs mHBB/PNLIP-RPS3A and mHBB/hHBA1, RM_61577 (SEQ ID NO: 307) and RM_61578 (SEQ ID NO: 308), respectively. Codon optimized SPCas9 ORFs were also generated with the GeneArt tool (Cas9-OPT2; SEQ ID NO: 72) and the maxiCAI method (Cas9-OPT4; SEQ ID NO: 73). Each of these ORFs was placed between the UTR pairs mHBB/PNLIP-RPS3A and mHBB/hHBA1 as RM_61579 (SEQ ID NO: 309) and RM_61580 (SEQ ID NO: 310), respectively, for Cas9-OPT2 and as RM_61581 (SEQ ID NO: 311) and RM_61582 (SEQ ID NO: 312), respectively, for Cas9-OPT4 (see table in FIG. 19A).

These mRNAs were assessed for their ability to knock out T Cell Receptor (TCR) α chain and β2-microglobulin in T cells when transfected along with appropriate single guide RNAs (sgRNA). For TCR α chain (TRAC) knock-out, a sgRNA was synthesized with target sequence of GUCUCUCAGCUGGUACA (SEQ ID NO: 313), and modifications of 2′-O-Methyl at 3 first and last bases, and 3′ phosphorothioate bonds between first 3 and last 2 bases (Synthego, CRISPRevolution sgRNA EZ kit-modified). A single guide RNA for β2-microglobulin (B2M) knock-out containing the target sequence of ACUCACGCUGGAUAGCCUCC (SEQ ID NO: 314) with the same modifications was also synthesized.

The SpCas9 mRNAs (approximately 4.7 kb) and the sgRNAs (100 nucleotides), were encapsulated in separate LNP formulations which were then further conjugated with anti-CD8 antibody. All tLNP were frozen until use.

T cells were isolated from fresh leukopaks with EasySep™ Human T Cell Isolation Kit (#100-0695, STEMCELL Technologies) following manufacturer's manual. Briefly, each leukopak was treated with ammonium chloride solution (#07800, STEMCELL Technologies) to lyse residual red blood cells and platelets and washed with EasySep™ Buffer (#20144, STEMCELL Technologies) with gentle centrifugations. The EasySep™ procedure involves magnetic beads and antibody complexes that recognize non-T cell antigens and T cells were isolated after removing non-T cells in a magnet (Easy 250 EasySep™ magnet, #100-0821, STEMCELL Technologies). Isolated T cells were frozen in CryoStor® CS10 (#210502, STEMCELL Technologies) and stored in LN2 for future use. T cell purity and phenotype were analyzed and confirmed by flow cytometry (Table 16).

TABLE 16
Phenotyping Antibody Panel

| | | | Antibody | | |
|---|---|---|---|---|---|
| Antigen | Clone | Fluorochrome | dilution | Company | Cat # |
| Live/Dead ™ Fixable | | AmCyan* | n/a | Invitrogen | L34957 |
| Aqua Dead Cell Stain | | | | | |
| Kit — | | | | | |
| CD4 | OKT4 | BV650 | 1:100 | Biolegend | 317436 |
| CD45 | HI30 | Qdot 800 (BV786) | 1:400 | Biolegend | 304048 |
| CD8 | HIT8a | FITC | 1:100 | Biolegend | 300906 |
| CD2 | RPA-2.10 | PE | 1:100 | Biolegend | 300208 |
| CD5 | 5D7 | APC | 1:100 | Thermo | MHCD0505 |
| CD3 | SP34-2 | APC-Cy7 | 1:100 | BD | BDB557757 |

*to discriminate live versus dead cells

Three days prior to transfection with the tLNP, isolated T cells from two donors were thawed and placed into culture in complete T cell media containing OpTmizer™ T-cell Expansion basal Medium (A10485, Gibco), OpTimizer™ T-cell Expansion Supplement (A10484, Gibco), human serum heat-inactivated (HP1022H1, Valley Biomed), GlutaMax™ Supplement (35050061, Gibco), Pen/Strep (1514032, Gibco), and human IL-2 (202-IL, R&D). Immediately after thawing, T cells were activated with DynaBeads™ Human T-activator CD3/CD28 for T cell Expansion and Activation (#11161D, ThermoFisher) at 1:1 (bead:cell) ratio for 3 days. On the day of tLNP transfection, activated T cells were first de-beaded using Easy 250 EasySep™ magnet (#100-0821, STEMCELL Technologies) and resuspended in complete T cell media at 1×10⁶ cells/mL. Cells were further seeded in to 96-well plates at 200 μL/well (2×10⁵ cells/well) prior to tLNP transfection. To maintain cell culture, T cells were passaged at 2×10⁵ to 1×10⁶/mL with complete T cell media every 2-3 days.

On the day of experiment, CD8-targeted tLNPs were thawed and equilibrated to room temperature on the benchtop. Once completely thawed, tLNPs were diluted in sterile water for injection. Amounts of tLNPs that provided 0 (that is, no tLNP), 0.1 ug (0.05 ug mRNA-tLNP plus 0.05 ug gRNA-tLNP), 0.3 ug (0.15 ug mRNA-tLNP plus 0.15 ug gRNA-tLNP), and 1.0 μg (0.5 ug mRNA-tLNP plus 0.5 ug gRNA-tLNP) were introduced to cells seeded in 96-well plate at 1×10⁶ cells/mL. Transfected T cells were passaged and maintained in complete T cell media at 2×10⁵ to 1×10⁶/mL for 7 days before collection for knock-out analysis.

Seven days after tLNP transfection, T cells were collected and stained with Aqua Live/Dead (#L34965, Invitrogen), anti-CD3-APC/Cy7 (BDB55775, BD), anti-CD4-BV650 (317436, BioLegend), anti-CD8-FITC (300906, BioLegend), and anti-B2M-PE (395704, BioLegend), and analyzed by multicolor flow cytometry. In the absence of TCR α chain, CD3 was not expressed at the cell surface. Thus knock-out of TRAC was assessed by staining for CD3. And the knock-out of B2M was evaluated by B2M staining.

The base construct, RM-61101, was able to mediate about 45% to 65% knock out of CD3 expression and about 25% to 45% for B2M for the RNA dosages of 0.1 to 1.0 μg. The results from the 0.3 and 1.0 μg doses of the other mRNAs normalized to the base mRNA are depicted in FIG. 19A. The amount of knock out obtained is dependent on several factors in addition to expression level of Cas9. Nonetheless, the tested constructs were generally able to increase the amount of knock out and the larger doses were generally more effective, consistent with an increase in Cas9 expression. The increased effect of the improved mRNAs was generally less apparent at the highest dose tested (that is, while the absolute effect is greater at the highest dose, the fold-change over the base construct is less than for the lower dose) (FIG. 19A). The results for the lowest dosage were more variable and can reflect a threshold where factors other than Cas9 expression level are swamping any signal from an improved Cas9 mRNA. The greater degree and consistency of effect observed for B2M knock out as compared to CD3 knock out can reflect a better sgRNA for the former and/or simply that there was more room for increased knock out as the base mRNA mediated substantially less B2M knock out than CD3 knock out.

These mRNAs were also assessed for their ability to knock out β2-microglobulin in hematopoietic stem and progenitor cells (HSPC) when transfected along with the sgRNA targeting B2M. The mRNA and sgRNA were formulated in separate tLNP in which the targeting moiety was an anti-CD117 antibody.

HSPCs were prepared as Plexifor-mobilized human peripheral blood (mPB) CD34+ cells obtained from STEMCELL Technologies (70075.1). A day before tLNP transfection, CD34+ cells were thawed in base media containing X-VIVO 10 (BP04-743Q, Lonza), human AB serum (HP1022, Valley Biomed), Flt3-L (130-096-479, Miltenyi), TPO (130-095-745, Miltenyi), IL-3 (130-095-069, Miltenyi), UM729 (72332, STEMCELL Technologies) and SR1 (72344, STEMCELL Technologies).

On the day of experiment, CD117-targeted tLNPs were thawed and equilibrated to room temperature on the benchtop. Once completely thawed, tLNPs were diluted in sterile water for injection. mRNA-tLNP and gRNA-tLNP were provided at 3-to-1 w/w ratio. Amounts of total tLNPs that provided 0 (that is, no tLNP), 1 μg, 3 μg, and 9 μg of total RNA, at a ratio of 0.75 μg mRNA-tLNP to 0.25 μg gRNA-tLNP per μg of total RNA, were introduced to cells seeded in 96-well plate at 1×10⁶ cells/mL. Transfected CD34+ cells were passaged and maintained in StemSpan SFEM II media (09655, STEMCELL Technologies) with StemSpan CD34+ Expansion Supplement (02691, STEMCELL Technologies) for 7 days before collection for knock-out analysis.

Seven days after tLNP transfection, CD34+ cells were collected and stained with Aqua Live/Dead (#L34965, Invitrogen), anti-CD34-AF488 (343518, BioLegend), and anti-B2M-PE (395704, BioLegend), and analyzed by multicolor flow cytometry.

The base construct, RM-61101, was able to mediate about 20% to 55% knock out B2M expression for the RNA dosages of 1 to 9 μg. The results from the other mRNAs normalized to the base mRNA are depicted in FIG. 19B. A very clear RNA dosage effect is seen consistent with increased Cas9 expression contributing to this result. Both codon optimization and UTR selection contributed to the improved performance of these mRNAs in both cell types.

In conclusion, FIG. 19A and FIG. 19B showed that the framework comprising the codon optimization methods and the 5′ UTR/3′ UTR pairs improved the performance of the mRNA construct independent of the protein encoded by the ORF.

### Example 13: CAR7 and CAR22 Expression on Expanded T Cells Via tLNP-Mediated mRNA Transfection In Vitro

To demonstrate the generality of improving mRNAs as described herein, various ORFs encoding different anti-CD20 CAR constructs, namely CAR7 and CAR22, were tested.

Base constructs of CAR7 and CAR22 utilized the TEV 5′ UTR and the XBG 3′ UTR and were codon optimized using the OPT6 strategy for CAR7 and OPT8 for CAR22 creating RM_61508 and RM_61511, respectively. ORF sequences for the improved mRNAs were as taken from the published literature for the CAR components (with the exception of the (G₄S)₃ linker in CAR7 which was encoded according to OPT2) or codon optimized with OPT4 and placed between the top performing 5′ UTR and 3′ UTR pairs, mHBB/PNLIP-RPS3A and mHBB/hHBA1-2× miR122 bs, from the evaluation of CAR1 and CAR2 constructs.

Expanded T cells (0.2 million) from two donors were transfected in vitro with 5 anti-CD20 CAR22 mRNA constructs or 5 anti-CD20 CAR7 mRNA formulated with CD8-targeted tLNP that provided 0.6 ug mRNA. CAR expression from each construct was assessed in each of the two donors in duplicate, Results are reported as CAR molecules per cell of total CD8+T cells at 24 hours post transfection (FIG. 20). CAR expression was measured by flow cytometry using anti-G4S antibody which detects the linker peptide within the scFv portion of the CAR.

Data indicated the improved CAR22 expression levels were two and three-fold than the base CAR22 construct, RM_61511. The data indicated the improved CAR7 expression levels were around ten-fold over that from base CAR7 construct, RM_61508.

CAR expression was detected using an anti-G4S antibody. CAR22 is based on Leu16 binder with linker peptide: GSTSGGGSGGGSGGGGSS (SEQ ID NO: 229) and CAR7 is based on 2.1.2 binder with linker peptide: GGGGSGGGGSGGGGS (SEQ ID NO: 252). Therefore, an anti-G4S antibody might have different binding strength for those two linker peptide sequences so that the observed CAR expression levels may not be directly comparable between CAR7 and CAR22.

The best performing mRNA improvement techniques that emerged from the evaluation of CAR1 and CAR2 mRNA constructs worked well with further CAR constructs and were not limited to anti-CD19 CAR constructs.

### Example 14: In Vitro Assessment of B Cell Killing, T Cells Activation, and Induced Cytokine Release of mRNA Constructs Encoding Anti-CD20 CAR7 and CAR22

To evaluate ability of the transfected improved mRNA constructs encoding anti-CD20 CAR7 and CAR22 to mediate activation of the transfected T cells and ability to kill B cells compared to the base constructs. In addition, cytokine release upon stimulation by transfected cells was also assessed.

Human PBMC (0.4 million) from two donors were transfected in vitro with five CAR22- and five CAR7-encoding mRNA constructs encapsulated in CD8-targeted tLNP that provided 0.2 μg, 0.6 μg, and 2 μg mRNA. Both CAR22 and CAR7 are anti-CD20 CARs. Transfected CD8+T cells expressed the anti-CD20 CAR payload and were cytotoxic against primary B cells present in the PBMC. B cell population was measured by flow cytometry with anti-CD19 antibody staining and B cell number was counted by adding counting beads during flow cytometry staining and acquisition. B cell number per μL was determined at 72 hours post transfection.

CAR-T cells were stimulated upon antigen engagement and expressed activation markers CD69 and CD25 as evaluated by flow cytometry with staining by anti-CD69 and anti-CD25 antibodies. CAR-T cells also secreted cytokines such as TNFα following stimulation by antigen engagement. TNFα was measured by ELISA assay using ELISA MAX™ Deluxe Set Human TNF-α kit (Biolegend) on cell supernatant collected at 48 hours after transfection.

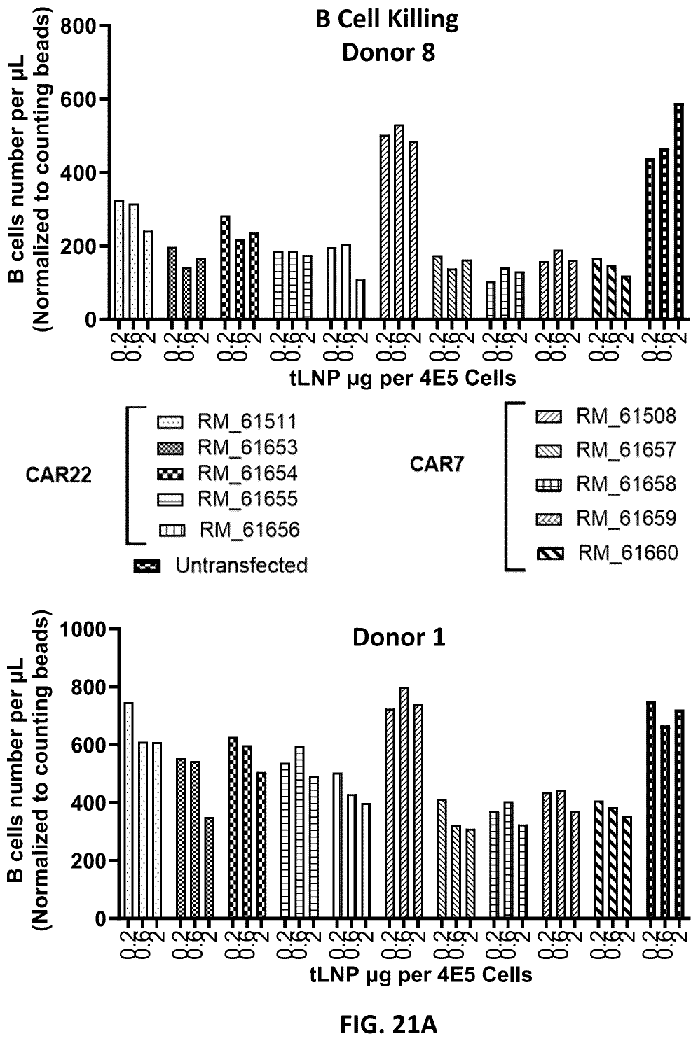

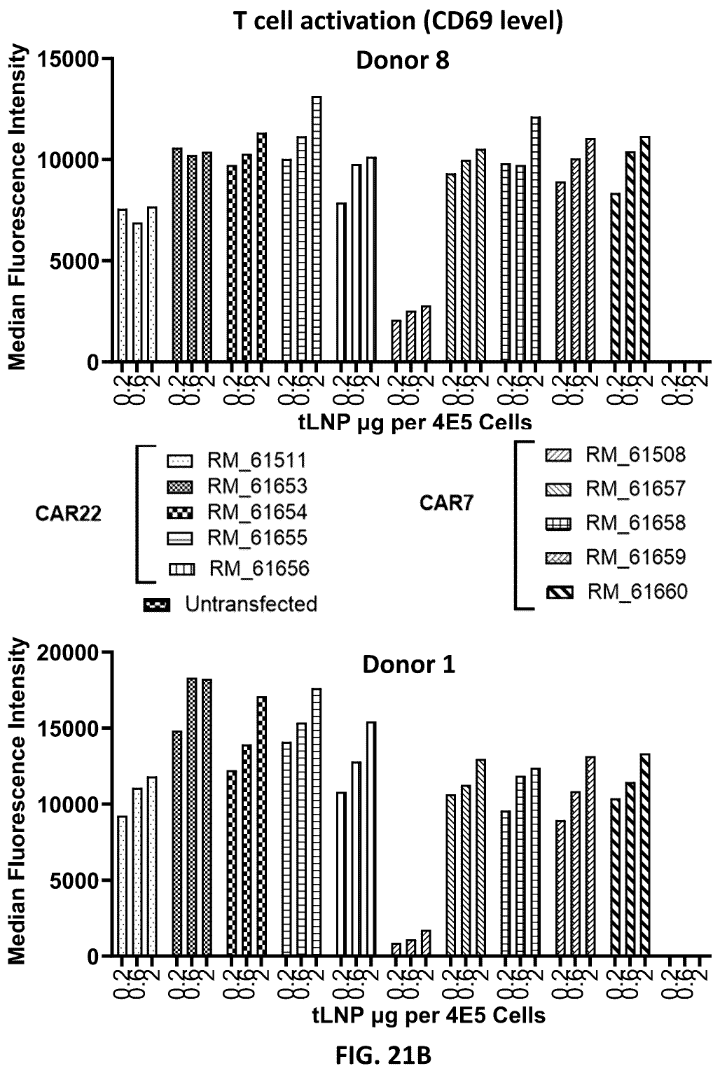

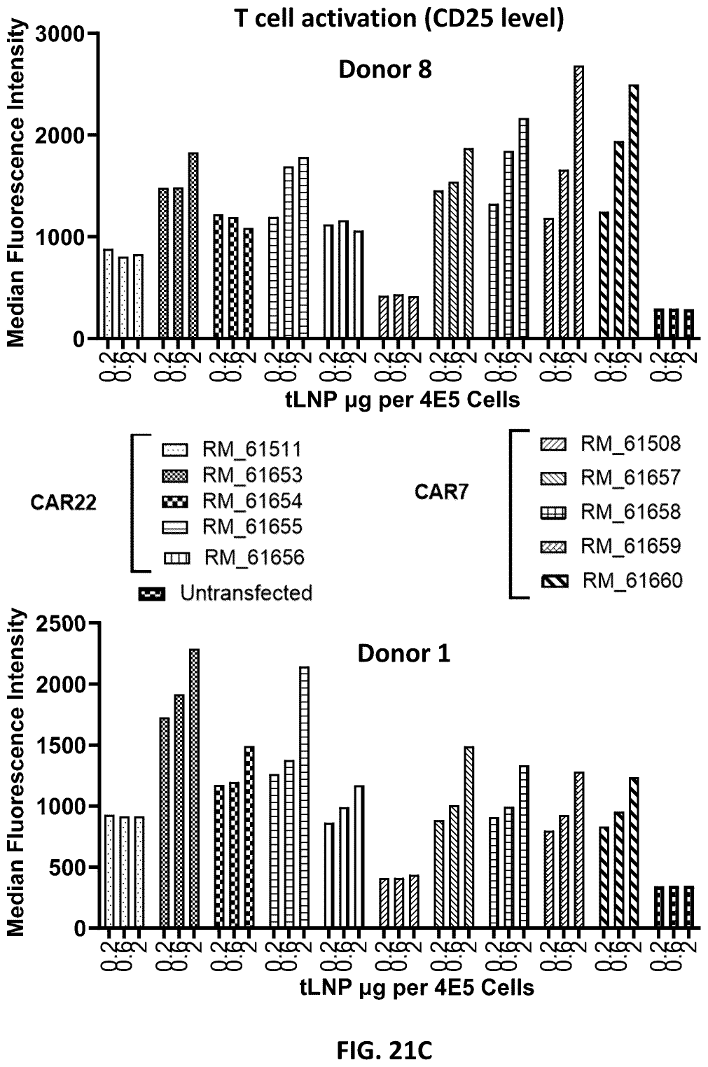

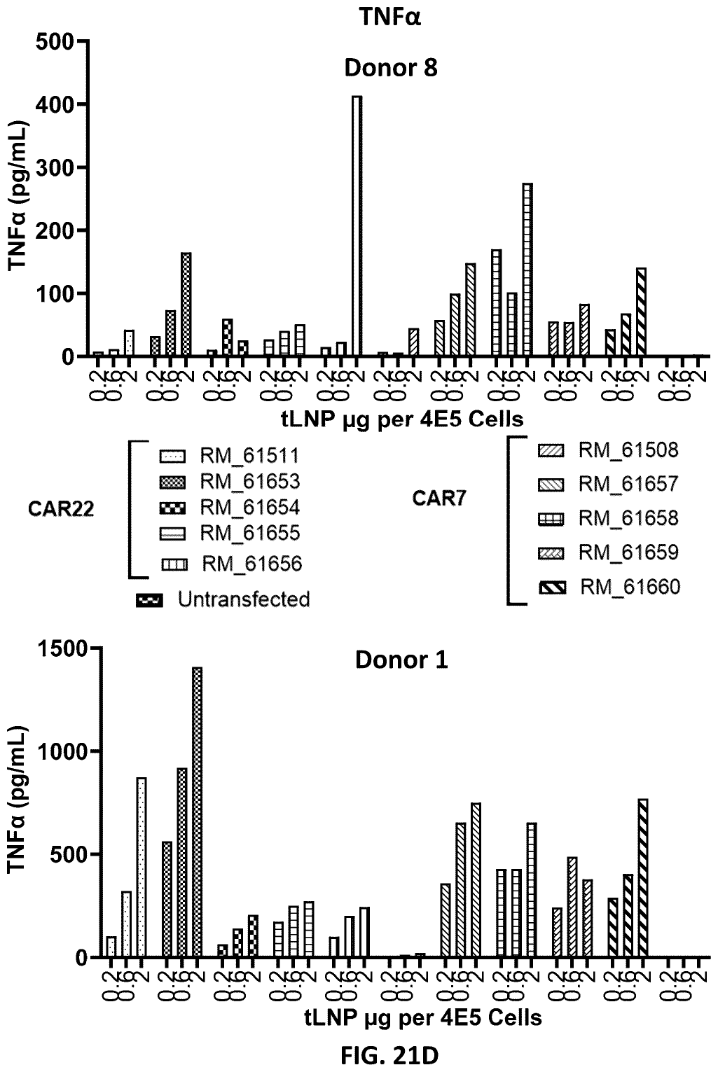

Data showed that all of the mRNAs except base CAR7 construct, RM_61508, reduced B cell number compared to untransfected control. The improved CAR7 constructs increased the CAR cytotoxicity as they reduced B cell number more than base CAR7 construct, RM_61508 (FIG. 21A). The improved CAR22 constructs exhibited less dramatic increases in B cell depletion similar to less dramatic increases in CAR expression achieved by these constructs. Data indicated that all tLNP transfected CD8+ T cells were CD69 positive compared to untransfected control which was not. The improved CAR7 and CAR22 mRNAs led to higher CD69 expression level than the base CARs, RM_61508 and RM-61511, respectively, although again the difference between base and improved CAR7 mRNAs was more dramatic (FIG. 21B). Data indicated that all tLNP transfected CD8+ T cells had increased CD25 expression compared to untransfected control. The improved CAR7 and CAR22 mRNAs led to higher CD25 expression level than their base constructs, RM_61508 and RM_61511, respectively (FIG. 21C). Data indicated that all tLNP transfected CD8+ T cells were making more TNFα compared to untransfected control. The optimized CAR7 was making more TNFα compared to the base CAR7 construct, RM_61508 (FIG. 21D). Of the CAR22-expressing CAR-T cells only RM_61653 consistently showed greater TNFα production, but this reflects in part the anomalously high production of TNFα by Donor 1 with the base construct, RM_61511 (FIG. 21D, lower panel).

The improved mRNA constructs encoding CAR7 and CAR22 were significantly effective in B cell killing, T cells activation, and T cell cytokine release.

### Example 15: In Vivo Assessment of B Cell Depletion and CAR Expression of mRNA Construct Encoding Anti-CD19 CAR2 in PBMC Engrafted NSG (NSG-PBMC) Mouse Model

To evaluate the transfection efficiency and the ability of T cells transfected with an mRNA construct encoding an anti-CD19 CAR2 to kill B cells in vivo, CD8-targeted F9 tLNP encapsulating mCherry mRNA or RM_61461 mRNA (herein after tLNP-98-mCherry and tLNP-98219, respectively) were injected intravenously into NSG immunodeficient mice engrafted with human PBMCs (tLNP-98mCherry treated or tLNP-98219 treated mice). This allowed characterization of the kinetics of T cell engineering and B cell depletion following treatment.

Immunodeficient NSG mice were injected with 10 million human PBMCs per animal. After 20 days of engraftment mice were evaluated for frequency of human CD45+ cells in circulation and staged in groups with similar averages. Day 21 post PBMC transfer, mice were dosed with a single intravenous (IV) injection via the tail vein of 30 μg/animal tLNP-98-mCherry mRNA or tLNP-98219. Mice were sacrificed at different time points after dosing and their spleens harvested to assess transfection and B cell depletion.

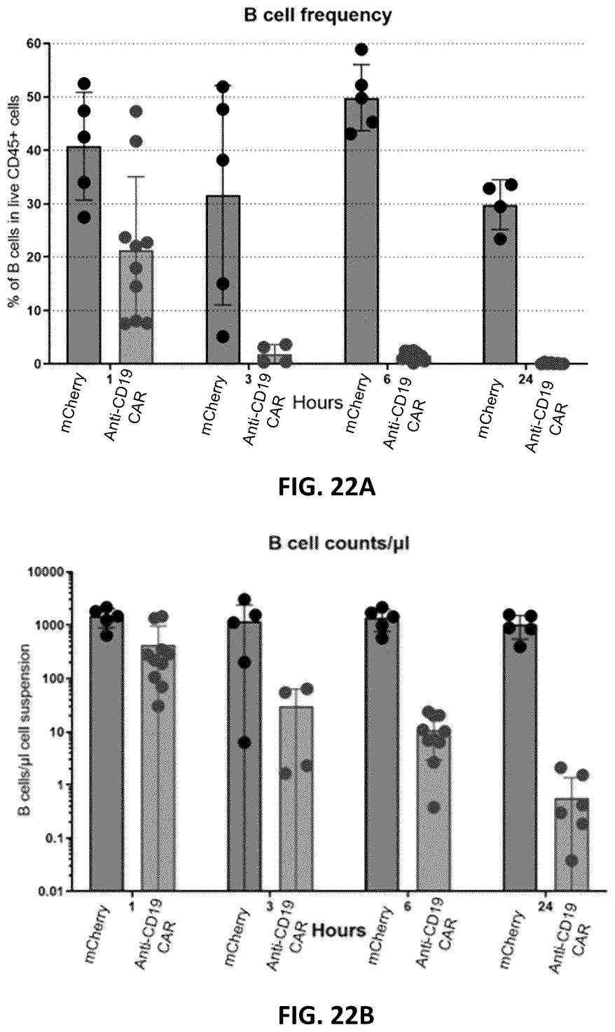

Average human B cell frequencies (as a percent of human immune cells [CD45+]) in the spleens of mCherry treated mice at 1-, 3—, 6-, and 24-hours post treatment were between 30-50% (FIG. 22A). The total number of human B cells per microliter of sample (FIG. 22B) remained at an average of 1000 human B cells across all time points for the mice receiving tLNP-98-mCherry. In contrast, tLNP-98219 treated mice demonstrated rapidly decreasing human B cell frequencies over time, with a mean value of 22% human B cells observed at 1-hour post treatment (FIG. 22A), followed by almost complete human B cell depletion at 3-, 6-, and 24-hours post treatment. Similar findings were observed in the analysis of the total number of B cells per microliter of sample (FIG. 22B) where approximately 500 human B cells/μL were observed at 1 hour post treatment, followed by approximately 10-30 B cells/μL at 3- and 6-hours post treatment. The lowest total cell counts were observed at 24 hours post treatment, with ≥2 human B cells/μL in all mice. The decrease in B cell numbers observed in tLNP-98219 treated mice indicated that expression of anti-CD19 CAR in CD8+ cells was responsible for B cell depletion.

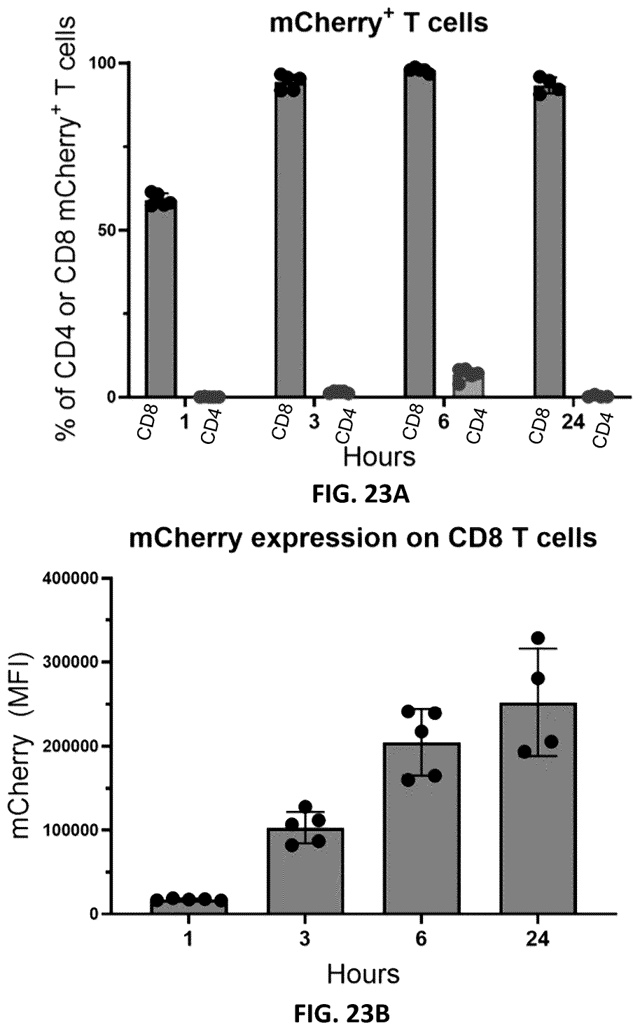

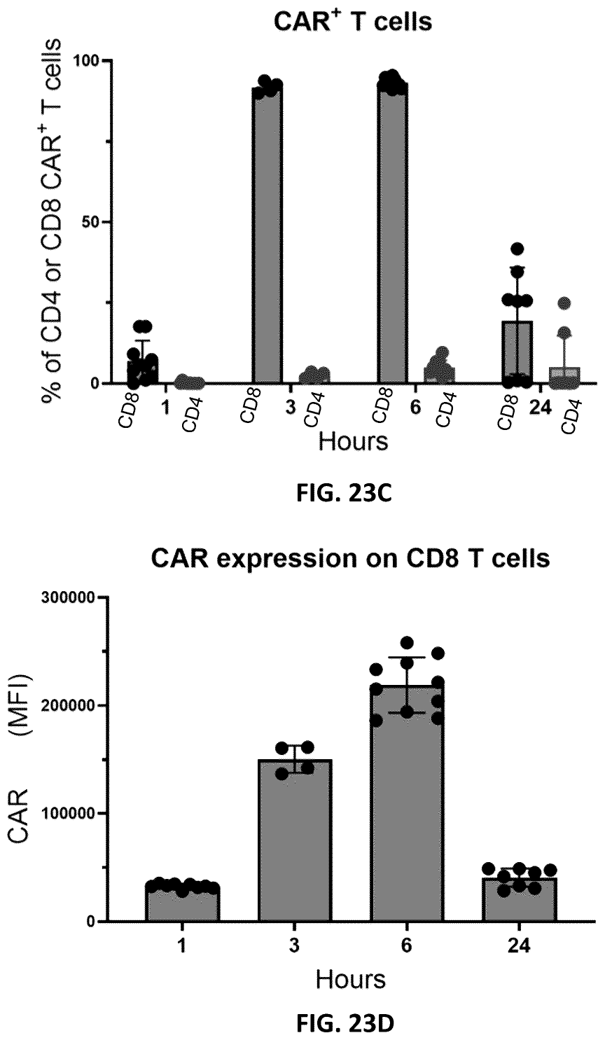

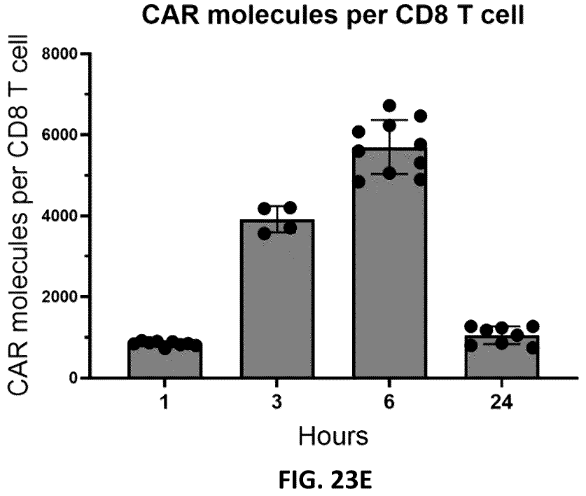

Expression of mCherry and CAR in splenic CD8+ T cells was evaluated. mCherry was expressed by 60% of the CD8+ T cells in mice treated with mCherry tLNP as early as 1 hour post treatment, and 97% of CD8+ T cells at 3 hours post treatment. The frequency of CD8+ T cells expressing mCherry remained at similarly high levels for 6- and 24-hours post treatment (FIG. 23A). Although this plateau was observed in the frequency of mCherry+ CD8+ T cells, the magnitude of mCherry expression, as measured by mean fluorescence intensity (MFI) (FIG. 23B), increased across time points.

In contrast, the frequency of human CD8+ T cells expressing the anti-CD19 CAR in mice treated with tLNP-98219 was approximately 6% at 1-hour post treatment, but then increased to 85% and 95% at 3- and 6-hours post treatment, respectively (FIG. 23C). Anti-CD19 CAR2 expression then dropped at 24-hours post treatment with a range from 0-40% of the CD8+ T cells expressing the CAR (FIG. 23C). Correlating with these observations, expression of the anti-CD19 CAR as assessed by MFI demonstrated an increase of expression from 1- to 6-hours post treatment (peaking at 6 hours), with a decrease of expression at 24-hours post treatment (FIG. 23D). Quantification of CAR molecules per CD8+ T cell indicated that at the peak of expression in this study (6-hours post treatment), the number of anti-CD19 CAR molecules per CD8+ T cell were within the range of about 5000 and 7000 molecules per cell (FIG. 23E).

Minimal expression of mCherry and CAR2 was observed in CD4+ cells, indicating specific CD8 cell targeting efficiency of the tLNP. mCherry expression in CD4 T cells averaged 0.02% at 1 hour post treatment, and increased to 1.4% average at 3 hours, peaking at 6.88% 6 hours post treatment. Finally, at 24 hours post treatment mCherry expression returned to low levels with an average of 0.09% of CD4 T (FIG. 23A).

CAR2 expression in spleen CD4 T cells was hardly detected 1 hour post treatment with only one mouse with 1% of CD4 T cells expressing the CAR. This was followed with a slight peaking at 6 hours post treatment where CAR was detected in a range between 1.8 and 9.6% of the CD4 CAR T cells. At 24 hours post treatment, 5 out of 10 mice presented lower than 0.2% of CAR+ CD4 T cells with two mice out of ten with 24.8 and 15.7% of CAR+ CD4 T cells. The averages CAR+ CD 4 T cells per time point were 0.196% at 1 hour post treatment, 2.52% at 3 hours, 4.96% at 6 hours and 5.89% 24 hours post treatment (FIG. 23C).

Expression of mCherry and CAR2 in CD4 T cells could not be evaluated in MFI terms nor molecules per cell due to the low number of CD4 T cells that were positive on each group (Less than 100 CAR2 or mCherry positive cells in the majority of treated animals).

The data obtained in this study indicated that tLNP-98219 can induce B cell depletion in the spleen of NSG-PBMC mice as early as 3 hours post treatment, with nearly all human B cells depleted from the spleen by 24 hours aftertreatment. Both tLNP-98219 and tLNP-mCherry showed preferential transfection of CD8⁺ T cells compared to CD4⁺ T cells. CAR expression in human CD8⁺ T cells peaked at 6 hours post treatment, with more than 90% of all CD8⁺ T cells in the spleen expressing the human anti-CD19 CAR with an average of 5000 to 7000 CAR molecules per cell. The data also showed that human anti-CD19 CAR expression is markedly reduced 24 hours post treatment, possibly due to interactions between the CAR and its cognate antigen.

### Example 16: Determination of Minimum Effective Dosage of tLNP Encapsulating Anti-CD19 CAR mRNA In Vivo

To evaluate the minimum effective dosage of tLNP-98219, similar experiments described in Example 15 were carried out on NCG immunodeficient mice engrafted with human PBMCs, except that various dosages of tLNP-98219 were tested and the effects were measured at 6 hour post-treatment. 1.5 μg, 3 μg, 7.5 μg, 15 μg, and 45 μg dosages corresponded to about 0.075 mg/kg, about 0.15 mg/kg, about 0.375 mg/kg, about 0.75 mg/kg, and about 2.25 mg/kg, respectively, for mice of approximately 20 g. The dosages referred to the amount of mRNA being provided. The tLNPs were formulated with F9 lipid content.

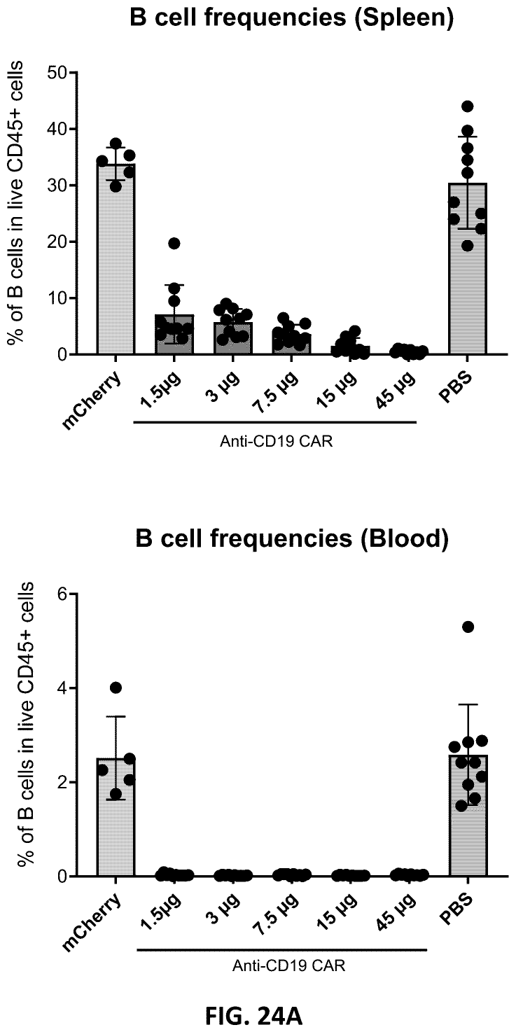

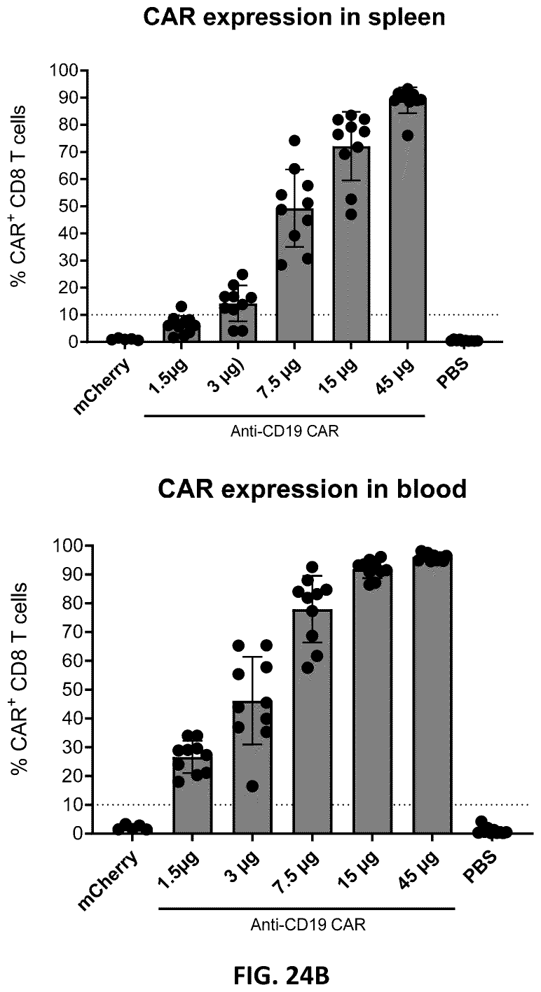

B cell frequencies at 6 hours post-treatment were equivalent in the PBS-treated and the mCherry-treated animals (FIG. 24A), indicating there was no impact of the tLNP delivery vehicle. Treatment with tLNP-98219 at any of the doses induced a complete depletion of all B cells in the blood. Analysis of the spleen, where there is higher B cell engraftment than that observed in the blood, demonstrated a dose response to tLNP-98219, with an almost complete B cell depletion in the tissue at doses of 15 μg and 45 μg per animal. These doses are the equivalent of 0.75 mg/kg and 2.25 mg/kg, respectively. In animals dosed with 7.5 μg and 3 μg, some B cells were still present in the spleen, but the B cell frequency remained below 10% of human CD45+ cells, compared to the mCherry and PBS controls with an average of 30-35% B cells. Finally, the lowest dose of 1.5 μg per animal (0.075 mg/kg) also showed a significant reduction, and only 2 out of 10 mice had B cells above 10% in the human CD45+ compartment.

In parallel, CAR expression was evaluated in CD8 T cells (FIG. 24B). In the tLNP-98219-treated groups, the frequency of CAR+ CD8 T cells was higher in the blood than in the spleen for all doses. However, in both blood and spleen, the frequency of CAR+ CD8 T cells demonstrated a dose dependency with higher engineering rates correlating with increased doses. The engineering rates in the blood ranged from 30% at the 1.5 μg dose, to nearly 100% at the 45 μg dose, of CD8 T cells expressing CAR in blood.

The data obtained in this study indicate that a CD8-targeted tLNP encapsulating mRNA construct encoding anti-CD19 CAR2, tLNP-98219 can induce B cell aplasia in humanized NCG-PBMC mice 6 hours post-treatment with doses as low as 1.5 μg per mouse (approximately 0.075 mg/kg). Frequency of CAR expression and depletion of splenic B cells was dose dependent in the tested dosage range.

### Example 17: Determination of Effective Dose Regimen of tLNP Encapsulating Anti-CD19 CAR mRNA In Vivo

To identify an effective dose regimen of tLNP-98219, similar experiments described in Example 15 were carried out, except with NSG immunodeficient mice engrafted with CD34+ human cells (purchased from Jackson Laboratory) and with a different dose and various dose regimens. Mice were treated intravenously (IV) with tLNP-98219, tLNP-982520 (anti-CD20 CAR), or tLNP-mCherry (control tLNP) either daily, every two days, or every three days, for a total of three treatments and 30 μg per animal per treatment. Starting at 24 hours post final treatment, and then weekly thereafter, peripheral bleeds were performed to evaluate the frequency of circulating human B cells.

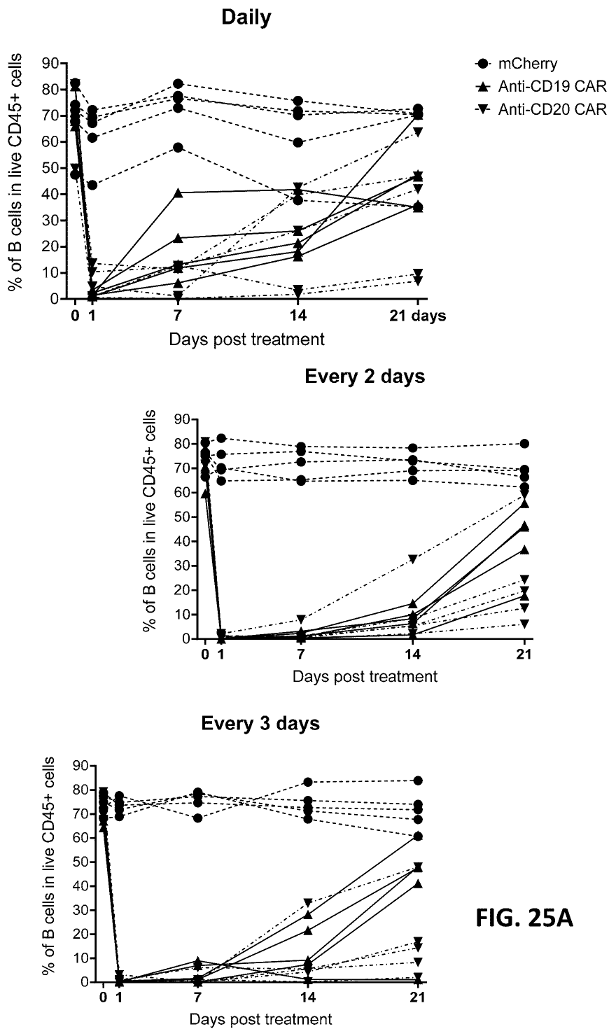

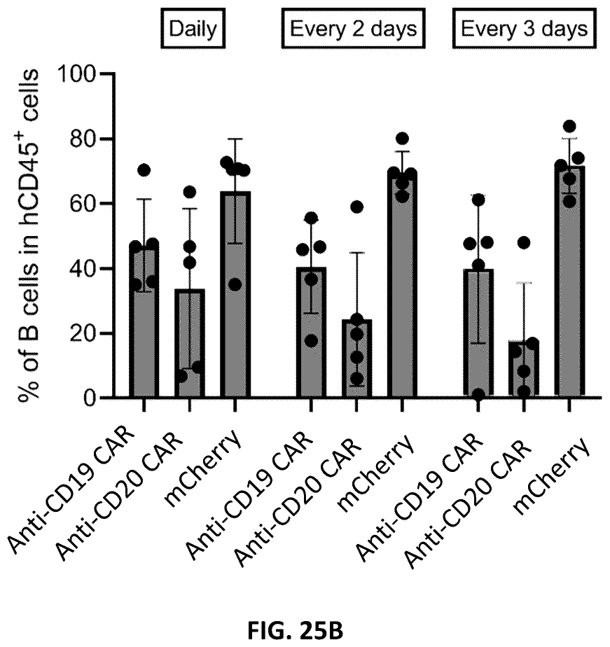

B cell frequencies were monitored longitudinally by flow cytometry in the blood of treated mice. All dose regimens with tLNP-98219 or tLNP-982520 induced a near complete B cell depletion 24 hours post third dose; there was no B cell depletion with any dose regimen with tLNP-mCherry (as expected). Treatment with tLNP-98219 or tLNP-982520 every two or three days resulted in a complete elimination of B cells from the peripheral circulation (FIG. 25A). Only the daily dosing of tLNP-98219 resulted in a complete elimination of B cells following daily dosing, whereas B cells were detectable in some of the tLNP-982520 treated mice.

Evaluation of the recovery of the circulating B cell frequencies demonstrated a marked increase at Day 7 post-treatment following daily dosing with either tLNP-98219 or tLNP-982520. In contrast, recovery of B cells following dosing every 2 or 3 days with the same test articles was limited at Day 7 post-treatment and only evident at Day 14 post-treatment (FIG. 25A). At Day 21 post-treatment, B cell frequencies remained lower than tLNP-mCherry-treated animals, particularly in those animals dosed every 2 or 3 days, with a trend towards lower B cell frequencies in tLNP-982520 (anti-CD20 CAR) treated mice (FIG. 25B).

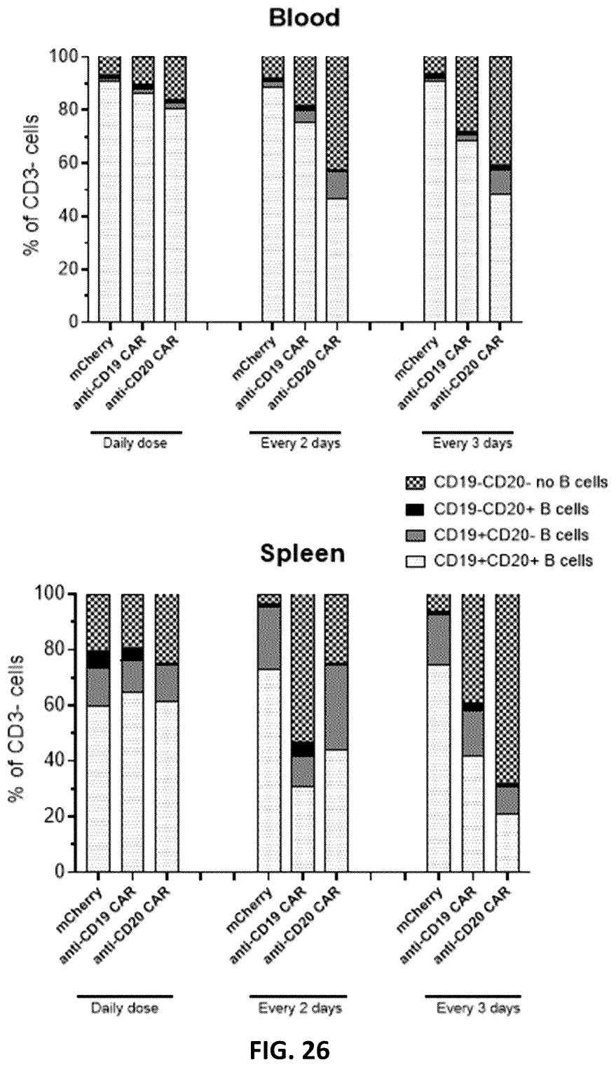

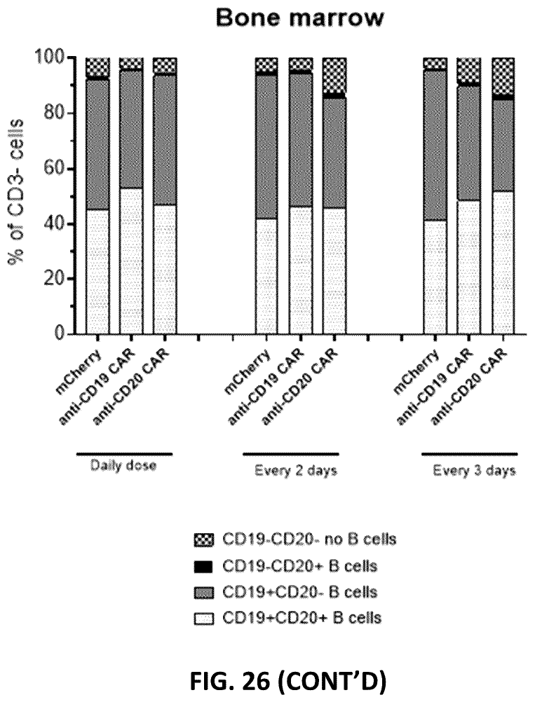

To exclude the possibility that targeting CD19 or CD20 led to differences in the re-populating B cell immunophenotype, the cell composition of the CD3− population in blood and tissues were evaluated at the terminal time point (Day 21 post treatment). Gating the human CD45+CD3− cells, CD19 and CD20 were used to define four subsets, namely, CD19+CD20− B cells, CD19+CD20+ B cells, CD19−CD20+ B cells, and non-B cells (CD19−CD20−) (FIG. 26). Analysis of the blood demonstrated that the repopulating B cells were predominantly a CD19+CD20+ immunophenotype. In the spleen, there was a larger population of CD19+CD20− B cells, but no consistent differences were observed between treatments. Almost equivalent proportions of CD19+CD20+ and CD19+CD20− B cells were observed in the bone marrow across all treatments. Therefore, differences in B cell repopulation were unlikely due to differences in the B cell immunophenotype of the repopulating cells.

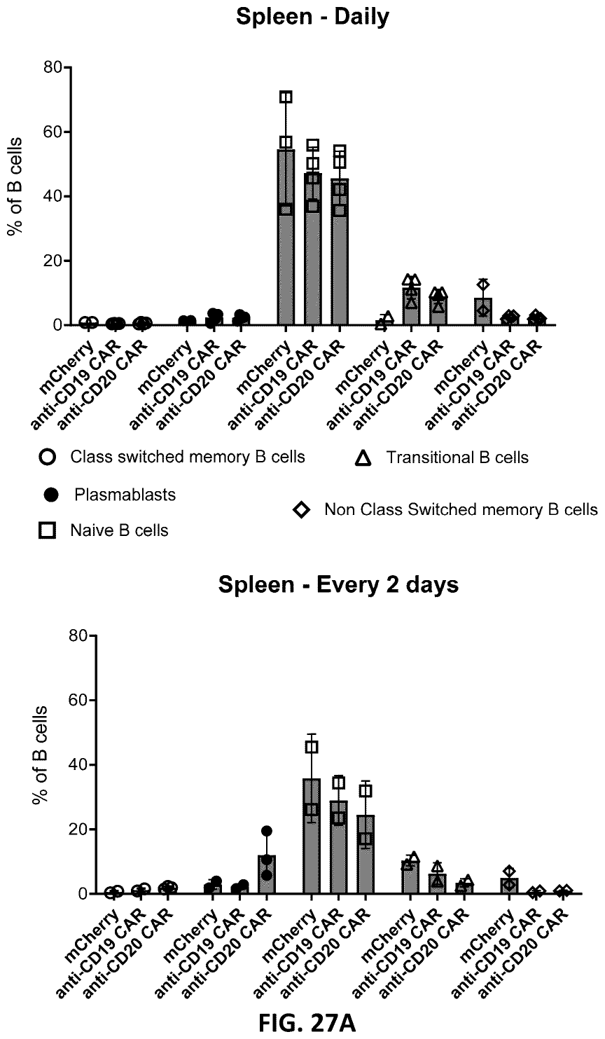

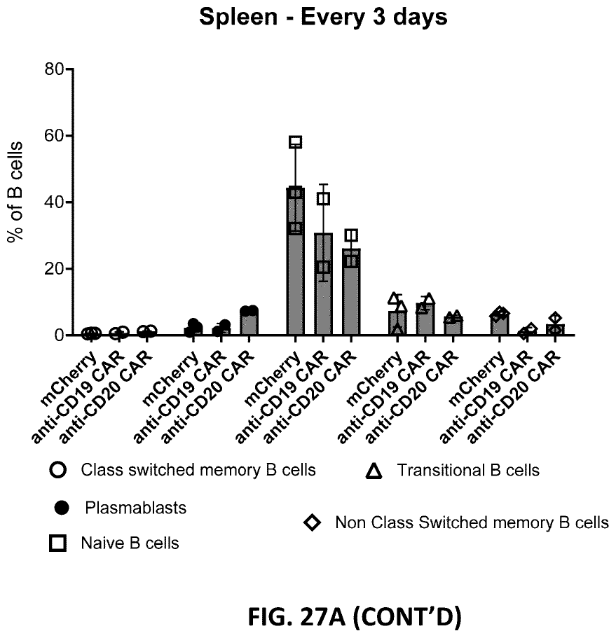

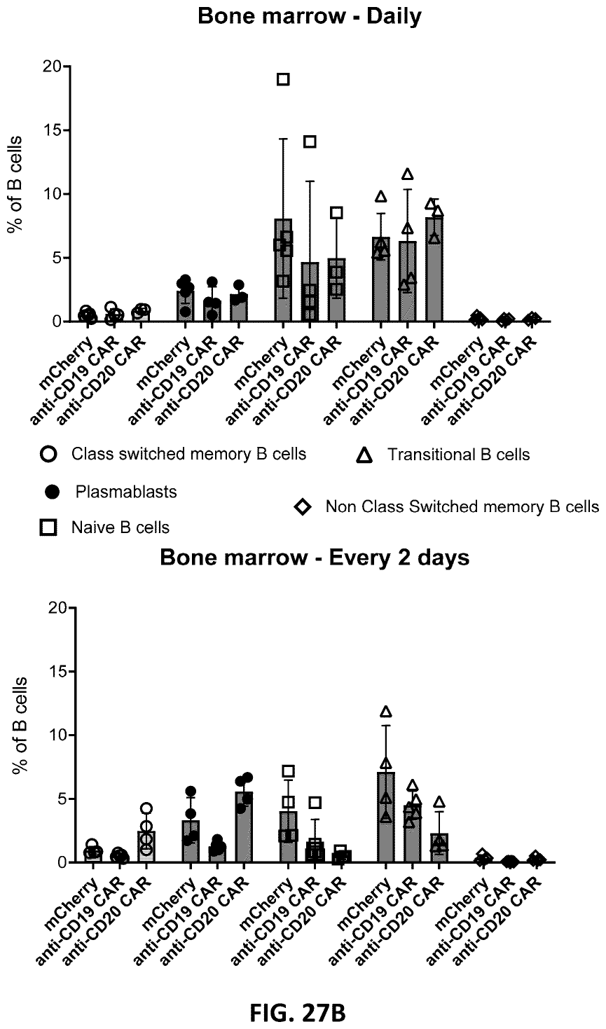

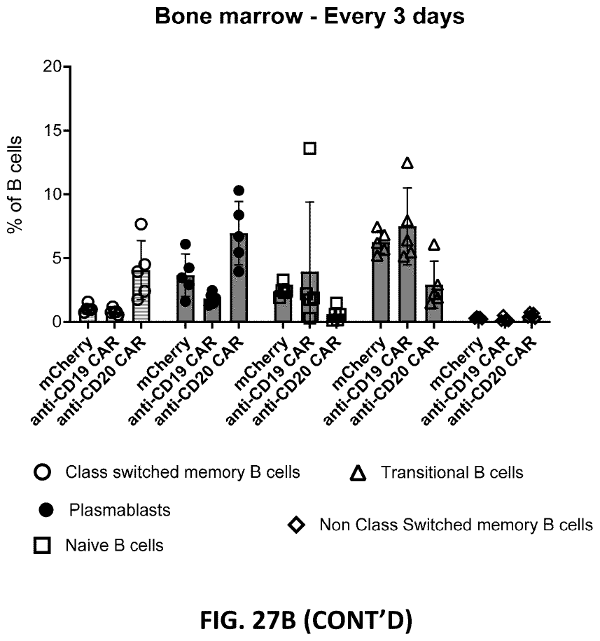

B cells subsets, including plasmablasts, naïve, transitional, and non-class-switched memory, were evaluated at Day 21 post-treatment in blood, spleen, and bone marrow using the flow cytometry. These subsets were defined as follows: B cells in the human CD45⁺ subpopulation were separated into three subsets after CD19⁺ cells were gated. The three subsets were determined by CD27 and IgD expression: CD27⁺IgD⁻, CD20⁺IgD⁺ (non-class switched memory B cells), and CD27⁻IgD⁺; from the CD27⁺IgD⁻ population, plasmablasts (CD20⁻CD38⁺) and class-switched memory B cells (CD20⁺CD38⁺); and from the CD27⁻IgD⁺ population, B cells were further divided into naïve B cells (CD24⁻CD38⁻) and transitional B cells (CD24⁺CD38⁺) based on the expression of CD24 and CD38. Analysis of blood B cell subsets was excluded due to the low number of B cells (CD19⁺) present in the blood at the time of the analysis, where almost all mice treated with tLNP-98219 and tLNP-982520 presented less than 500 B cells (CD19⁺) per sample, the cutoff for analysis. In the spleen, naïve B cells were the major B cell subset, and no major differences were observed between treatments (FIG. 27A). In the bone marrow, the biggest B cell population had a CD19⁺CD27⁻IgD⁻ immunophenotype, which is of unclear function in this model, and therefore not classified within any of the evaluated subsets (data not shown). Within the evaluated subsets in the bone marrow, a reduction in plasmablasts was observed in mice treated with tLNP-98219 every 2 or 3 days, but not with tLNP-982520. Conversely, a reduction in transitional B cells was observed in the bone marrow of mice treated with tLNP-982520 every 2 or 3 days, but not with tLNP-98219 (FIG. 27B).

The data obtained in this study indicated that B cell depletion is more durable in this mouse model following tLNP treatment cycles of every 2 or 3 days with tLNP-98219 that targets CD19 on B cells or tLNP-982520 that targets CD20 on B cells compared to daily treatment with the same tLNPs. This correlated with observed higher CAR expression in CD8⁺ T cells in mice dosed with tLNP-98219 or tLNP-982520 every 2 or 3 days (data now shown). While B cell subsets in this model were largely of a naïve immunophenotype, differences were observed in B cell subsets in the bone marrow following treatment with tLNP-98219 or tLNP-982520 every 2 or 3 days. The reduction of plasmablasts in tLNP-98219-treated mice, or transitional B cells in tLNP-982520-treated mice, likely reflected a difference in CD19 and CD20 expression in the B cell subsets.

### Example 18: In Vivo Assessment of Tumor Cell Killing by CD8-Targeted tLNP Encapsulating mRNA Encoding Anti-CD19 CAR

To assess the tumor cell killing of anti-CD19 CAR T cells upon treatment with tLNP-98219, the NALM6 tumor model was utilized. NSG mice were engrafted with NALM6-tumor cells and human PBMCs, including T cells that could be targeted and engineered in vivo. After PBMC engraftment, tumors were implanted and allowed to grow for 7 days before the mice were imaged and randomized into groups by tumor size. Mice were then injected with 10 μg or 30 μg tLNP-98219 or tLNP-mCherry twice a week for a total of 5 doses.

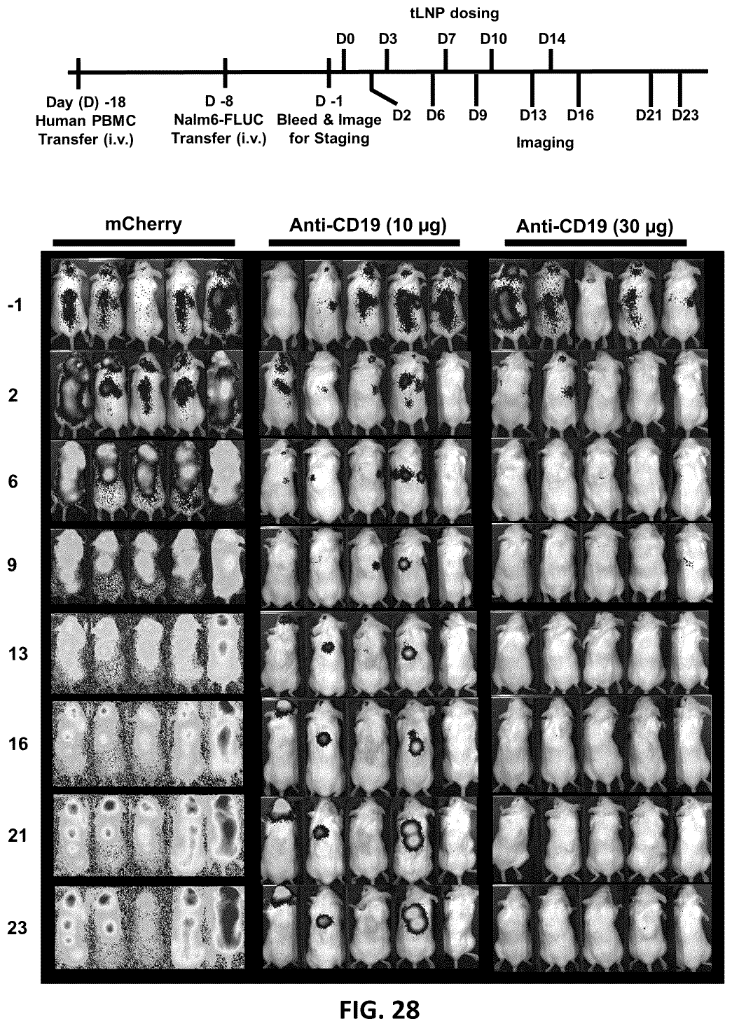

The 10 μg dose of tLNP-98219 resulted in significant tumor control compared to the control group (FIG. 28)). 30 μg dosing resulted in near complete clearance of the tumor in 4/5 animals by two days after the first dose, and in 5F5 animals by 3 days after the second dose (Day 6; FIG. 28).

TABLE 17
Humanized anti-CD8A whole antibody and related sequences.

| | | | SEQ |
|---|---|---|---|
| | | | ID |
| Name | Note | Sequence | NO |
| CBD1017vh | VH of mouse anti- | EVQLQQSGPELVKPGASVKISCKASRYTFTD | 180 |
| | CD8A antibody | YNLHWVKLSHEKSLEWIGFIYPYNGGTGYNQ | |
| | clone CT8 | KFKNKAKLTVDYSSSTAYMELRSLTSVDAAV | |
| | | YYCARDHRYNEGVSFDYWGQGTTLTVSS | |
| VH-CDR1 | VH-CDR1 of | RYTFTDYNLH | 181 |
| CBD1017 | mouse anti-CD8A | | |
| | antibody clone | | |
| | CT8 | | |
| VH-CDR2 | VH-CDR2 of | FIYPYNGGTG | 182 |
| CBD1017 | mouse anti-CD8A | | |
| | antibody clone | | |
| | CT8 | | |
| VH-CDR3 | VH-CDR3 of | DHRYNEGVSFDY | 183 |
| CBD1017 | mouse anti-CD8A | | |
| | antibody clone | | |
| | CT8 | | |
| CBD1017vl | VL of mouse anti- | NIVLTQSPASLAVSLGQRATISCRASESVDG | 184 |
| | CD8A antibody | FGNSFMNWYQQKPGQSPKLLIYLASNLESGV | |
| | clone CT8 | PARFSGSGSRTDFTLTIDPVEADDAATYYCQ | |
| | | QNNEDPYTFGGGTKLEIK | |
| VL-CDR1 | VL-CDR1 of | RASESVDGFGNSFMN | 185 |
| CBD1017 | mouse anti-CD8A | | |
| | antibody clone | | |
| | CT8 | | |
| VL-CDR2 | VL-CDR2 of | LASNLES | 186 |
| CBD1017 | mouse anti-CD8A | | |
| | antibody clone | | |
| | CT8 | | |
| VL-CDR3 | VL-CDR3 of | QQNNEDPYT | 187 |
| CBD1017 | mouse anti-CD8A | | |
| | antibody clone | | |
| | CT8 | | |
| 1-46*01 | IgHV1-46*01/ | QVQLVQSGAEVKKPGASVKVSCKASGYTFTS | 188 |
| | IGHJ6*01 | YYMHWVRQAPGQGLEWMGIINPSGGSTSYAQ | |
| | germline VH | KFQGRVTMTRDTSTSTVYMELSSLRSEDTAV | |
| | | YYCARWGQGTTVTVSS | |
| h1017-H1 | Humanized CT8 | QVQLVQSGAEVKKPGASVKVSCKASRYTFTD | 189 |
| | VH variant based | YNLHWVRQAPGQGLEWMGFIYPYNGGTGYAQ | |
| | on VH1-46 | KFQGRVTMTRDTSTSTVYMELSSLRSEDTAV | |
| | | YYCARDHRYNEGVSFDYWGQGTTVTVSS | |
| h1017-H2 | Humanized CT8 | QVQLVQSGAEVKKPGASVKVSCKASRYTFTD | 190 |
| | VH variant based | YNLHWVRQAPGQGLEWMGFIYPYNGGTGYAQ | |
| | on VH1-46 | KFQGRVTMTVDTSTSTAYMELSSLRSEDTAV | |
| | | YYCARDHRYNEGVSFDYWGQGTTVTVSS | |
| h1017-H3 | Humanized CT8 | QVQLVQSGAEVKKPGASVKISCKASRYTFTD | 191 |
| | VH variant based | YNLHWVRQAPGQGLEWIGFIYPYNGGTGYAQ | |
| | on VH1-46 | KFQGRATLTVDTSTSTAYMELSSLRSEDTAV | |
| | | YYCARDHRYNEGVSFDYWGQGTTLTVSS | |
| h1017-H4 | Humanized CT8 | QVQLVQSGAEVKKPGASVKVSCKASRYTFTD | 192 |
| | VH variant based | YNLHWVRQAPGQGLEWMGFIYPYNGGTGYAQ | |
| | on VH1-46 | KFQGRVTMTVDYSTSTAYMELSSLRSEDTAV | |
| | | YYCARDHRYNEGVSFDYWGQGTTVTVSS | |
| h1017-H5 | Humanized CT8 | QVQLVQSGAEVKKPGASVKISCKASRYTFTD | 193 |
| | VH variant based | YNLHWVRQAPGQGLEWIGFIYPYNGGTGYAQ | |
| | on VH1-46 | KFQGRATLTVDYSTSTAYMELSSLRSEDTAV | |
| | | YYCARDHRYNEGVSFDYWGQGTTLTVSS | |
| 1-39*01 | IgKV1-39*01/ | DIQMTQSPSSLSASVGDRVTITCRASQSISS | 194 |
| | IGKJ2*01 | YLNWYQQKPGKAPKLLIYAASSLQSGVPSRF | |
| | germline VL | SGSGSGTDFTLTISSLQPEDFATYYCQQSYS | |
| | | TPPFGQGTKLEIK | |
| h1017-L1 | Humanized CT8 | DIQMTQSPSSLSASVGDRVTITCRASESVDG | 195 |
| | VL variant based | FGNSFMNWYQQKPGKAPKLLIYLASNLESGV | |
| | on VK1-39 | PSRFSGSGSGTDFTLTISSLQPEDFATYYCQ | |
| | | QNNEDPYTFGQGTKLEIK | |
| h1017-L2 | Humanized CT8 | DIQLTQSPSSLSASVGDRATITCRASESVDG | 196 |
| | VL variant based | FGNSFMNWYQQKPGKAPKLLIYLASNLESGV | |
| | on VK1-39 | PSRFSGSGSGTDFTLTISSVQPEDFATYYCQ | |
| | | QNNEDPYTFGQGTKLEIK | |
| h1017-L3 | Humanized CT8 | DIQLTQSPSSLSASVGDRATITCRASESVDG | 197 |
| | VL variant based | FGNSFMNWYQQKPGKAPKLLIYLASNLESGV | |
| | on VK1-39 | PSRFSGSGSRTDFTLTISSVQPEDFATYYCQ | |
| | | QNNEDPYTFGQGTKLEIK | |
| VH hOKT8 | VH of humanized | EVQLVQSGAEVKKPGASVKVSCKASGFNIKD | 198 |
| | mouse anti-CD8A | TYIHWVRQAPGQGLEWIGRIDPANDNTLYAS | |
| | antibody clone | KFQGRATITADTSTSTAYLELSSLRSEDTAV | |
| | OKT8 | YYCGRGYGYYVFDHWGQGTLVTVSS | |
| VL hOKT8 | VL of humanized | DVQITQSPSSLSASVGDRVTITCRTSRSISQ | 199 |
| | mouse anti-CD8A | YLAWYQEKPGKTNKLLIYSGSTLQSGIPSRF | |
| | antibody clone | SGSGSGTDFTLTISSLQPEDFATYYCQQVNE | |
| | OKT8 | FPPTFGQGTKVEIK | |
| VH OKT8 | VH of mouse anti- | EVQLQQSGAELVKPGASVKLSCTASGFNIKD | 200 |
| | CD8A antibody | TYIHFVRQRPEQGLEWIGRIDPANDNTLYAS | |
| | clone OKT8 | KFQGKATITADTSSNTAYMHLCSLTSGDTAV | |
| | | YYCGRGYGYYVFDHWGQGTTLTVSS | |
| VL OKT8 | VL of mouse anti- | DVQINQSPSFLAASPGETITINCRTSRSISQ | 201 |
| | CD8A antibody | YLAWYQEKPGKTNKLLIYSGSTLQSGIPSRF | |
| | clone OKT8 | SGSGSGTDFTLTISGLEPEDFAMYYCQQHNE | |
| | | NPLTFGAGTKLELK | |
| VH | N55S of h1017-H2 | QVQLVQSGAEVKKPGASVKVSCKASRYTFTD | 202 |
| CBD1380 | | YNLHWVRQAPGQGLEWMGFIYPYSGGTGYAQ | |
| | | KFQGRVTMTVDTSTSTAYMELSSLRSEDTAV | |
| | | YYCARDHRYNEGVSFDYWGQGTTVTVSS | |
| VH | N55Q of h1017-H2 | QVQLVQSGAEVKKPGASVKVSCKASRYTFTD | 203 |
| CBD1381 | | YNLHWVRQAPGQGLEWMGFIYPYQGGTGYAQ | |
| | | KFQGRVTMTVDTSTSTAYMELSSLRSEDTAV | |
| | | YYCARDHRYNEGVSFDYWGQGTTVTVSS | |
| VH | N55A of h1017-H2 | QVQLVQSGAEVKKPGASVKVSCKASRYTFTD | 204 |
| CBD1382 | | YNLHWVRQAPGQGLEWMGFIYPYAGGTGYAQ | |
| | | KFQGRVTMTVDTSTSTAYMELSSLRSEDTAV | |
| | | YYCARDHRYNEGVSFDYWGQGTTVTVSS | |
| VH | N55D of h1017-H2 | QVQLVQSGAEVKKPGASVKVSCKASRYTFTD | 205 |
| CB1383 | | YNLHWVRQAPGQGLEWMGFIYPYDGGTGYAQ | |
| | | KFQGRVTMTVDTSTSTAYMELSSLRSEDTAV | |
| | | YYCARDHRYNEGVSFDYWGQGTTVTVSS | |
| Mod 1-18 | Modified IgHV1- | QVQLVQSGAEVKKPGASVKVSCKASGYTFTS | 206 |
| | 18*01 germline | YYMHWVRQAPGQGLEWMGIINPSGGSTSYAQ | |
| | VH | KFQGRVTMTRDTSTSTVYMELSSLRSEDTAV | |
| | | YYCARWGQGTLVTVS | |
| h1017-H6 | Humanized CT8 | QVQLVQSGAEVKKPGASVKVSCKASRYTFTD | 207 |
| | VH variant based | YNLHWVRQAPGQGLEWMGFIYPYNGGTNYAQ | |
| | on VH1-18 | KFQGRVTITADTSTSTAYMELSSLRSEDTAV | |
| | | YYCARDHRY | |
| h1017-H7 | Humanized CT8 | QVQLVQSGAEVKKPGASVKVSCKASRYTFTD | 208 |
| | VH variant based | YNLHWVRQAPGQGLEWIGFIYPYNGGTGYAQ | |
| | on VH1-18 | KFQGRVTITADTSTSTAYMELSSLRSEDTAV | |
| | | YYCARDHRYNEGVSFDYWGQGTLVTVSS | |
| h1017-H8 | Humanized CT8 | QVQLVQSGAEVKKPGASVKVSCKASRYTFTD | 209 |
| | VH variant based | YNLHWVRQAPGQGLEWIGFIYPYNGGTGYAQ | |
| | on VH1-18 | KFKGRVTITADTSTSTAYMELSSLRSEDTAV | |
| | | YYCARDHRYNEGVSFDYWGQGTLVTVSS | |
| h1017-H9 | Humanized CT8 | QVQLVQSGAEVKKPGASVKVSCKASRYTFTD | 210 |
| | VH variant based | YNLHWVRQAPGQGLEWMGFIYPYNGGTGYAQ | |
| | on VH1-18 | KFQGRVTLTVDYSTSTAYMELSSLRSEDTAV | |
| | | YYCARDHRYNEGVSFDYWGQGTLVTVSS | |
| h1017-H10 | Humanized CT8 | QVQLVQSGAEVKKPGASVKVSCKASRYTFTD | 211 |
| | VH variant based | YNLHWVRQAPGQGLEWIGFIYPYNGGTGYAQ | |
| | on VH1-18 | KFQGRVTLTVDYSTSTAYMELSSLRSEDTAV | |
| | | YYCARDHRYNEGVSFDYWGQGTLVTVSS | |
| Mod 3D-11 | Modified | EIVLTQSPGTLSLSPGERATLSCRASQSVSS | 212 |
| | IgKV3D-11*01 | SYLAWYQQKPGQAPRLLIYGASSRATGIPDR | |
| | germline VL | FSGSGSGTDFTLTISRLEPEDFAVYYCQQYG | |
| | | SSPLTFGQGTKVEIK | |
| h1017-L4 | Humanized CT8 | EIVLTQSPGTLSLSPGERATLSCRASESVDG | 213 |
| | VL variant based | FGNSFMNWYQQKPGQAPRLLIYLASNLESGI | |
| | on VK3D-11 | PARFSGSGSGTDFTLTISRLEPEDFAVYYCQ | |
| | | QNNEDPYTFGQGTKVEIK | |
| h1017-L5 | Humanized CT8 | EIVLTQSPGTLSLSPGERATLSCRASESVDG | 214 |
| | VL variant based | FGNSFMNWYQQKPGQAPRLLIYLASNLESGV | |
| | on VK3D-11 | PARFSGSGSGTDFTLTISRLEPEDFAVYYCQ | |
| | | QNNEDPYTFGQGTKVEIK | |
| h1017-L6 | Humanized CT8 | EIVLTQSPGTLSLSPGERATLSCRASESVDG | 215 |
| | VL variant based | FGNSFMNWYQQKPGQAPRLLIYLASNLESGV | |
| | on VK3D-11 | PARFSGSGSRTDFTLTISRLEPEDFAVYYCQ | |
| | | QNNEDPYTFGQGTKVEIK | |
| IGKC_human | Kappa chain | RTVAAPSVFIFPPSDEQLKSGTASVVCLLNN | 216 |
| | constant region | FYPREAKVQWKVDNALQSGNSQESVTEQDSK | |
| | used in the whole | DSTYSLSSTLTLSKADYEKHKVYACEVTHQG | |
| | antibody | LSSPVTKSFNRGEC | |
| | constructs | | |
| | (UniProt P01834) | | |
| IGG1_human | Wild type IgG1 | ASTKGPSVFPLAPSSKSTSGGTAALGCLVKD | 217 |
| | constant regions | YFPEPVTVSWNSGALTSGVHTFPAVLQSSGL | |
| | (UniProt P0DOX5) | YSLSSVVTVPSSSLGTQTYICNVNHKPSNTK | |
| | | VDKKVEPKSCDKTHTCPPCPAPELLGGPSVF | |
| | | LFPPKPKDTLMISRTPEVTCVVVDVSHEDPE | |
| | | VKFNWYVDGVEVHNAKTKPREEQYNSTYRVV | |
| | | SVLTVLHQDWLNGKEYKCKVSNKALPAPIEK | |
| | | TISKAKGQPREPQVYTLPPSRDELTKNQVSL | |
| | | TCLVKGFYPSDIAVEWESNGQPENNYKTTPP | |
| | | VLDSDGSFFLYSKLTVDKSRWQQGNVFSCSV | |
| | | MHEALHNHYTQKSLSLSPGK | |
| IgG1_human | Fc-silenced IgG1 | ASTKGPSVFPLAPSSKSTSGGTAALGCLVKD | 218 |
| LALAPA EM | constant regions | YFPEPVTVSWNSGALTSGVHTFPAVLQSSGL | |
| | used in the whole | YSLSSVVTVPSSSLGTQTYICNVNHKPSNTK | |
| | antibody | VDKKVEPKSCDKTHTCPPCPAPEAAGGPSVF | |
| | constructs (with | LFPPKPKDTLMISRTPEVTCVVVDVSHEDPE | |
| | E358 and M360 | VKFNWYVDGVEVHNAKTKPREEQYNSTYRVV | |
| | allotypic | SVLTVLHQDWLNGKEYKCKVSNKALAAPIEK | |
| | reversions) | TISKAKGQPREPQVYTLPPSREEMTKNQVSL | |
| | | TCLVKGFYPSDIAVEWESNGQPENNYKTTPP | |
| | | VLDSDGSFFLYSKLTVDKSRWQQGNVFSCSV | |
| | | MHEALHNHYTQKSLSLSPG | |
| IgG1_human | Fc-silenced IgG1 | ASTKGPSVFPLAPSSKSTSGGTAALGCLVKD | 219 |
| LALAPA DL | constant regions | YFPEPVTVSWNSGALTSGVHTFPAVLQSSGL | |
| | used in the whole | YSLSSVVTVPSSSLGTQTYICNVNHKPSNTK | |
| | antibody | VDKKVEPKSCDKTHTCPPCPAPELLGGPSVF | |
| | constructs (with | LFPPKPKDTLMISRTPEVTCVVVDVSHEDPE | |
| | D358 and L360 | VKFNWYVDGVEVHNAKTKPREEQYNSTYRVV | |
| | allotypic | SVLTVLHQDWLNGKEYKCKVSNKALPAPIEK | |
| | residues) | TISKAKGQPREPQVYTLPPSRDELTKNQVSL | |
| | | TCLVKGFYPSDIAVEWESNGQPENNYKTTPP | |
| | | VLDSDGSFFLYSKLTVDKSRWQQGNVFSCSV | |
| | | MHEALHNHYTQKSLSLSPGK | |
| VH-CDR1 | VH N33 | RYTFTDYX₁LH | 220 |
| N33var | degenerate | | |
| VH-CDR2 | VH N55 | FIYPYX₁GGTG | 221 |
| N55var1 | degenerate (set | | |
| | w/o D) | | |
| VH-CDR2 | VH N55 | FIYPYX₂GGTG | 222 |
| N55var1 | degenerate (set | | |
| | w/ D) | | |
| VH-CDR3 | VH N103 | DHRYX₁EGVSFDY | 223 |
| N103var | degenerate | | |
| VL-CDR1 | VL D30 and N34 | RASESVX₃GFGX₁SFMN | 224 |
| D30 and | degenerate | | |
| N34var | | | |
| VH-CDR2 | VL N57 | LASX₂LES | 225 |
| N57var | degenerate | | |
| VH-CDR3 | VL N95, N96, and | QQX₂X₂EX₃PYT | 226 |
| N95, N96, | D98 degenerate | | |
| and D98var | | | |
| h1017-1-46 | Humanized CT8 | QVQLVQSGAEVKKPGASVKVSCKASRYTFTD | 338 |
| (1) | VH based on | YX₁LHWVRQAPGQGLEWMGFIYPYX₁GGTGY | |
| | VH1-46 with CDR | AQKFQGRVTMTRDTSTSTVYMELSSLRSEDT | |
| | variants (1) | AVYYCARDHRYX₁EGVSFDYWGQGTTVTVSS | |
| h1017-1-46 | Humanized CT8 | QVQLVQSGAEVKKPGASVKVSCKASRYTFTD | 339 |
| (2) | VH based on | YX₁LHWVRQAPGQGLEWMGFIYPYX₂GGTGY | |
| | VH1-46 with CDR | AQKFQGRVTMTRDTSTSTVYMELSSLRSEDT | |
| | variants (2) | AVYYCARDHRYX₁EGVSFDYWGQGTTVTVSS | |
| h1017-1-39 | Humanized CT8 | DIQMTQSPSSLSASVGDRVTITCRASESVX₃ | 340 |
| | VL based on VK1- | GFGX₁SFMNWYQQKPGKAPKLLIYLASX₂LE | |
| | 39 with CDR | SGVPSRFSGSGSGTDFTLTISSLQPEDFATY | |
| | variants | YCQQX₂X₂EX₃PYTFGQGTKLEIK | |
| h1017-1-18 | Humanized CT8 | QVQLVQSGAEVKKPGASVKVSCKASRYTFTD | 341 |
| (1) | VH based on | YX₁LHWVRQAPGQGLEWMGFIYPYX₁GGTGY | |
| | VH1-18 with CDR | AQKFQGRVTMTRDTSTSTVYMELSSLRSEDT | |
| | variants | AVYYCARDHRYX₁EGVSFDYWGQGTLVTVS | |
| h1017-1-18 | Humanized CT8 | QVQLVQSGAEVKKPGASVKVSCKASRYTFTD | 342 |
| (2) | VH based on | YX₁LHWVRQAPGQGLEWMGFIYPYX₂GGTGY | |
| | VH1-18 with CDR | AQKFQGRVTMTRDTSTSTVYMELSSLRSEDT | |
| | variants | AVYYCARDHRYX₁EGVSFDYWGQGTLVTVS | |
| h1017-3D-11 | Humanized CT8 | EIVLTQSPGTLSLSPGERATLSCRASESVX₃ | 343 |
| | VL based on | GFGX₁SFMNWYQQKPGQAPRLLIYLASX₂LE | |
| | VK3D-11 with | SGIPDRFSGSGSGTDFTLTISRLEPEDFAVY | |
| | CDR variants | YCQQX₂X₂EX₃PYTFGQGTKVEIK | |
| VH-CDR2v2 | Modified VH- | FIYPYSGGTG | 344 |
| CBD1017 | CDR2 of mouse | | |
| N55S | anti-CD8A clone | | |
| | CT8 | | |
| VH-CDR2v3 | Modified VH- | FIYPYQGGTG | 345 |
| CBD1017 | CDR2 of mouse | | |
| N55Q | anti-CD8A clone | | |
| | CT8 | | |
| VH-CDR2v4 | Modified VH- | FIYPYAGGTG | 346 |
| CBD1017 | CDR2 of mouse | | |
| N55A | anti-CD8A | | |
| | antibody clone | | |
| | CT8 | | |
| CBD1033 HC | h1017-H2 Fc- | QVQLVQSGAEVKKPGASVKVSCKASRYTFTD | 347 |
| | silenced IgG1 | YNLHWVRQAPGQGLEWMGFIYPYNGGTGYAQ | |
| | | KFQGRVTMTVDTSTSTAYMELSSLRSEDTAV | |
| | | YYCARDHRYNEGVSFDYWGQGTTVTVSSAST | |
| | | KGPSVFPLAPSSKSTSGGTAALGCLVKDYFP | |
| | | EPVTVSWNSGALTSGVHTFPAVLQSSGLYSL | |
| | | SSVVTVPSSSLGTQTYICNVNHKPSNTKVDK | |
| | | KVEPKSCDKTHTCPPCPAPEAAGGPSVFLFP | |
| | | PKPKDTLMISRTPEVTCVVVDVSHEDPEVKF | |
| | | NWYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1033 LC | h1017-L2 Kappa | DIQLTQSPSSLSASVGDRATITCRASESVDG | 348 |
| CBD1035 LC | chain | FGNSFMNWYQQKPGKAPKLLIYLASNLESGV | |
| CBD1037 LC | | PSRFSGSGSGTDFTLTISSVQPEDFATYYCQ | |
| CBD1039 LC | | QNNEDPYTFGQGTKLEIKRTVAAPSVFIFPP | |
| CBD1380 LC | | SDEQLKSGTASVVCLLNNFYPREAKVQWKVD | |
| CBD1381 LC | | NALQSGNSQESVTEQDSKDSTYSLSSTLTLS | |
| CBD1382 LC | | KADYEKHKVYACEVTHQGLSSPVTKSFNRGE | |
| | | C | |
| VL CBD1443 | D30E of h1017-L2 | DIQLTQSPSSLSASVGDRATITCRASESVEG | 350 |
| VL CBD1446 | | FGNSFMNWYQQKPGKAPKLLIYLASNLESGV | |
| | | PSRFSGSGSGTDFTLTISSVQPEDFATYYCQ | |
| | | QNNEDPYTFGQGTKLEIK | |
| VL CBD1444 | D30S of h1017-L2 | DIQLTQSPSSLSASVGDRATITCRASESVSG | 351 |
| VL CBD1447 | | FGNSFMNWYQQKPGKAPKLLIYLASNLESGV | |
| VL CBD1575 | | PSRFSGSGSGTDFTLTISSVQPEDFATYYCQ | |
| VL CBD1576 | | QNNEDPYTFGQGTKLEIK | |
| VL CBD1622 | | | |
| VL CBD1623 | | | |
| VL CBD1445 | D30A of h1017-L2 | DIQLTQSPSSLSASVGDRATITCRASESVAG | 352 |
| VL CBD1448 | | FGNSFMNWYQQKPGKAPKLLIYLASNLESGV | |
| | | PSRFSGSGSGTDFTLTISSVQPEDFATYYCQ | |
| | | QNNEDPYTFGQGTKLEIK | |
| VH CBD1449 | N55S of h1017-H4 | QVQLVQSGAEVKKPGASVKVSCKASRYTFTD | 353 |
| | | YNLHWVRQAPGQGLEWMGFIYPYSGGTGYAQ | |
| | | KFQGRVTMTVDYSTSTAYMELSSLRSEDTAV | |
| | | YYCARDHRYNEGVSFDYWGQGTTVTVSS | |
| VH CBD1450 | N55Q of h1017-H4 | QVQLVQSGAEVKKPGASVKVSCKASRYTFTD | 354 |
| VH CBD1575 | | YNLHWVRQAPGQGLEWMGFIYPYQGGTGYAQ | |
| | | KFQGRVTMTVDYSTSTAYMELSSLRSEDTAV | |
| | | YYCARDHRYNEGVSFDYWGQGTTVTVSS | |
| VH CBD1451 | N55A of h1017-H4 | QVQLVQSGAEVKKPGASVKVSCKASRYTFTD | 355 |
| VH CBD1576 | | YNLHWVRQAPGQGLEWMGFIYPYAGGTGYAQ | |
| | | KFQGRVTMTVDYSTSTAYMELSSLRSEDTAV | |
| | | YYCARDHRYNEGVSFDYWGQGTTVTVSS | |
| CBD1035 HC | h1017-H3 Fc- | QVQLVQSGAEVKKPGASVKISCKASRYTFTD | 356 |
| | silenced IgG1 | YNLHWVRQAPGQGLEWIGFIYPYNGGTGYAQ | |
| | | KFQGRATLTVDTSTSTAYMELSSLRSEDTAV | |
| | | YYCARDHRYNEGVSFDYWGQGTTLTVSSAST | |
| | | KGPSVFPLAPSSKSTSGGTAALGCLVKDYFP | |
| | | EPVTVSWNSGALTSGVHTFPAVLQSSGLYSL | |
| | | SSVVTVPSSSLGTQTYICNVNHKPSNTKVDK | |
| | | KVEPKSCDKTHTCPPCPAPEAAGGPSVFLFP | |
| | | PKPKDTLMISRTPEVTCVVVDVSHEDPEVKF | |
| | | NWYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1039 HC | h1017-H5 Fc- | QVQLVQSGAEVKKPGASVKISCKASRYTFTD | 357 |
| CBD1040 HC | silenced IgG1 | YNLHWVRQAPGQGLEWIGFIYPYNGGTGYAQ | |
| | | KFQGRATLTVDYSTSTAYMELSSLRSEDTAV | |
| | | YYCARDHRYNEGVSFDYWGQGTTLTVSSAST | |
| | | KGPSVFPLAPSSKSTSGGTAALGCLVKDYFP | |
| | | EPVTVSWNSGALTSGVHTFPAVLQSSGLYSL | |
| | | SSVVTVPSSSLGTQTYICNVNHKPSNTKVDK | |
| | | KVEPKSCDKTHTCPPCPAPEAAGGPSVFLFP | |
| | | PKPKDTLMISRTPEVTCVVVDVSHEDPEVKF | |
| | | NWYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1040 LC | h1017-L3 Kappa | DIQLTQSPSSLSASVGDRATITCRASESVDG | 358 |
| | chain | FGNSFMNWYQQKPGKAPKLLIYLASNLESGV | |
| | | PSRFSGSGSRTDFTLTISSVQPEDFATYYCQ | |
| | | QNNEDPYTFGQGTKLEIKRTVAAPSVFIFPP | |
| | | SDEQLKSGTASVVCLLNNFYPREAKVQWKVD | |
| | | NALQSGNSQESVTEQDSKDSTYSLSSTLTLS | |
| | | KADYEKHKVYACEVTHQGLSSPVTKSFNRGE | |
| | | C | |
| CBD1380 HC | N55S of h1017-H2 | QVQLVQSGAEVKKPGASVKVSCKASRYTFTD | 359 |
| | Fc-silenced IgG1 | YNLHWVRQAPGQGLEWMGFIYPYSGGTGYAQ | |
| | | KFQGRVTMTVDTSTSTAYMELSSLRSEDTAV | |
| | | YYCARDHRYNEGVSFDYWGQGTTVTVSSAST | |
| | | KGPSVFPLAPSSKSTSGGTAALGCLVKDYFP | |
| | | EPVTVSWNSGALTSGVHTFPAVLQSSGLYSL | |
| | | SSVVTVPSSSLGTQTYICNVNHKPSNTKVDK | |
| | | KVEPKSCDKTHTCPPCPAPEAAGGPSVFLFP | |
| | | PKPKDTLMISRTPEVTCVVVDVSHEDPEVKF | |
| | | NWYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1381 HC | N55Q of h1017- | QVQLVQSGAEVKKPGASVKVSCKASRYTFTD | 360 |
| | H2 Fc-silenced | YNLHWVRQAPGQGLEWMGFIYPYQGGTGYAQ | |
| | IgG1 | KFQGRVTMTVDTSTSTAYMELSSLRSEDTAV | |
| | | YYCARDHRYNEGVSFDYWGQGTTVTVSSAST | |
| | | KGPSVFPLAPSSKSTSGGTAALGCLVKDYFP | |
| | | EPVTVSWNSGALTSGVHTFPAVLQSSGLYSL | |
| | | SSVVTVPSSSLGTQTYICNVNHKPSNTKVDK | |
| | | KVEPKSCDKTHTCPPCPAPEAAGGPSVFLFP | |
| | | PKPKDTLMISRTPEVTCVVVDVSHEDPEVKF | |
| | | NWYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1382 HC | N55A of h1017- | QVQLVQSGAEVKKPGASVKVSCKASRYTFTD | 361 |
| | H2 Fc-silenced | YNLHWVRQAPGQGLEWMGFIYPYAGGTGYAQ | |
| | IgG1 | KFQGRVTMTVDTSTSTAYMELSSLRSEDTAV | |
| | | YYCARDHRYNEGVSFDYWGQGTTVTVSSAST | |
| | | KGPSVFPLAPSSKSTSGGTAALGCLVKDYFP | |
| | | EPVTVSWNSGALTSGVHTFPAVLQSSGLYSL | |
| | | SSVVTVPSSSLGTQTYICNVNHKPSNTKVDK | |
| | | KVEPKSCDKTHTCPPCPAPEAAGGPSVFLFP | |
| | | PKPKDTLMISRTPEVTCVVVDVSHEDPEVKF | |
| | | NWYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1037 HC | h1017-H4 Fc- | QVQLVQSGAEVKKPGASVKVSCKASRYTFTD | 362 |
| | silenced IgG1 | YNLHWVRQAPGQGLEWMGFIYPYNGGTGYAQ | |
| | | KFQGRVTMTVDYSTSTAYMELSSLRSEDTAV | |
| | | YYCARDHRYNEGVSFDYWGQGTTVTVSSAST | |
| | | KGPSVFPLAPSSKSTSGGTAALGCLVKDYFP | |
| | | EPVTVSWNSGALTSGVHTFPAVLQSSGLYSL | |
| | | SSVVTVPSSSLGTQTYICNVNHKPSNTKVDK | |
| | | KVEPKSCDKTHTCPPCPAPEAAGGPSVFLFP | |
| | | PKPKDTLMISRTPEVTCVVVDVSHEDPEVKF | |
| | | NWYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |

HC: high chain.
LC: light chain.
X is any amino acid.
X1 is N, S, Q, or A.
X2 is N, Q, D, S, or A.
X3 is D, E, S, or A.

TABLE 18
Humanized anti-CD8A F(ab′)2 variants and classic and
engineered F(ab′) constant regions. Variable domains
are Bold. Constant regions are in regular fonts.
C-terminal hinge residue modification is Bold Italic
Underlined. The mutation sites of native disulfide
residues (to remove the cysteine used in the native
disulfide) are Italic Underlined. The engineered
disulfide residues (mutation to introduce a new
cysteine for interchain disulfide bond) are Bold
Underlined. The target conjugation sites are Underlined.
Kabat numbering is used to identify the position of
substitutions and other modifications of the amino
acid sequences.

| Name | LC | HC |
|---|---|---|
| IgG1 F(ab′) | | ASTKGPSVFPLAPSSKSTSGGT |
| | | AALGCLVKDYFPEPVTVSWNSG |
| | | ALTSGVHTFPAVLQSSGLYSLS |
| | | SVVTVPSSSLGTQTYICNVNHK |
| | | PSNTKVDKKVEPKSCDKTHTCP |
| | | PCPAPELLG (SEQ ID NO: |
| | | 363) |
| CBD1033.6 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQESVT | APSSKSTSGGTAALGCLVKDYF |
| | EQDSKDSTYSLSSTLTLSKADYEK | PEPVTVSWNSGALTSGVHTFPA |
| | HKVYACEVTHQGLSSPVTKSFNRG | VLQSSGLYSLSSVVTVPSSSLG |
| | EC (SEQ ID NO: 364) | TQTYICNVNHKPSNTKVDKKVE |
| | | PKSCDKTHTCPPCPAPELLG |
| | | (SEQ ID NO: 365) |
| IgG4 F(ab′) | | ASTKGPSVFPLAPCSRSTSEST |
| | | AALGCLVKDYFPEPVTVSWNSG |
| | | ALTSGVHTFPAVLQSSGLYSLS |
| | | SVVTVPSSSLGTKTYTCNVDHK |
| | | PSNTKVDKRVESKYGPPCPPCP |
| | | APEFLG (SEQ ID NO: |
| | | 366) |
| CBD1033.61 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQESVT | APCSRSTSESTAALGCLVKDYF |
| | EQDSKDSTYSLSSTLTLSKADYEK | PEPVTVSWNSGALTSGVHTFPA |
| | HKVYACEVTHQGLSSPVTKSFNRG | VLQSSGLYSLSSVVTVPSSSLG |
| | EC (SEQ ID NO: 364) | TKTYTCNVDHKPSNTKVDKRVE |
| | | SKYGPPCPPCPAPEFLG (SEQ |
| | | ID NO: 367) |
| Truncated | | ASTKGPSVFPLAPSSKSTSGGT |
| P245 IgG1 | | AALGCLVKDYFPEPVTVSWNSG |
| F(ab′) | | ALTSGVHTFPAVLQSSGLYSLS |
| | | SVVTVPSSSLGTQTYICNVNHK |
| | | PSNTKVDKKVEPKSCDKTHTCP |
| | | PCPAP (SEQ ID NO: 368) |
| CBD1033.9 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQESVT | APSSKSTSGGTAALGCLVKDYF |
| | EQDSKDSTYSLSSTLTLSKADYEK | PEPVTVSWNSGALTSGVHTFPA |
| | HKVYACEVTHQGLSSPVTKSFNRG | VLQSSGLYSLSSVVTVPSSSLG |
| | EC (SEQ ID NO: 364) | TQTYICNVNHKPSNTKVDKKVE |
| | | PKSCDKTHTCPPCPAP (SEQ |
| | | ID NO: 369) |
| Truncated | | ASTKGPSVFPLAPCSRSTSEST |
| P245 IgG4 | | AALGCLVKDYFPEPVTVSWNSG |
| F(ab′) | | ALTSGVHTFPAVLQSSGLYSLS |
| | | SVVTVPSSSLGTKTYTCNVDHK |
| | | PSNTKVDKRVESKYGPPCPPCP |
| | | AP (SEQ ID NO: 370) |
| CBD1033.10 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQESVT | APCSRSTSESTAALGCLVKDYF |
| | EQDSKDSTYSLSSTLTLSKADYEK | PEPVTVSWNSGALTSGVHTFPA |
| | HKVYACEVTHQGLSSPVTKSFNRG | VLQSSGLYSLSSVVTVPSSSLG |
| | EC (SEQ ID NO: 364) | TKTYTCNVDHKPSNTKVDKRVE |
| | | SKYGPPCPPCPAP (SEQ ID |
| | | NO: 371) |
| Truncated | | ASTKGPSVFPLAPSSKSTSGGT |
| P241, P240A | | AALGCLVKDYFPEPVTVSWNSG |
| and P241A | | ALTSGVHTFPAVLQSSGLYSLS |
| IgG1 F(ab′) | | SVVTVPSSSLGTQTYICNVNHK |
| | | PSNTKVDKKVEPKSCDKTHTCA |
| | | A (SEQ ID NO: 372) |
| CBD1033.40 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQESVT | APSSKSTSGGTAALGCLVKDYF |
| | EQDSKDSTYSLSSTLTLSKADYEK | PEPVTVSWNSGALTSGVHTFPA |
| | HKVYACEVTHQGLSSPVTKSFNRG | VLQSSGLYSLSSVVTVPSSSLG |
| | EC (SEQ ID NO: 364) | TQTYICNVNHKPSNTKVDKKVE |
| | | PKSCDKTHTCAA (SEQ ID |
| | | NO: 373) |
| Truncated | | ASTKGPSVFPLAPCSRSTSEST |
| P241, P240A | | AALGCLVKDYFPEPVTVSWNSG |
| and P241A | | ALTSGVHTFPAVLQSSGLYSLS |
| IgG4 F(ab′) | | SVVTVPSSSLGTKTYTCNVDHK |
| | | PSNTKVDKRVESKYGPPCAA |
| | | (SEQ ID NO: 374) |
| CBD1033.12 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQESVT | APCSRSTSESTAALGCLVKDYF |
| | EQDSKDSTYSLSSTLTLSKADYEK | PEPVTVSWNSGALTSGVHTFPA |
| | HKVYACEVTHQGLSSPVTKSFNRG | VLQSSGLYSLSSVVTVPSSSLG |
| | EC (SEQ ID NO: 364) | TKTYTCNVDHKPSNTKVDKRVE |
| | | SKYGPPCAA (SEQ ID NO: |
| | | 375) |
| S162C and | RTVAAPSVFIFPPSDEQLKSGTAS | |
| C214S | VVCLLNNFYPREAKVQWKVDNALQ | |
| Kappa chain | SGNSQECVTEQDSKDSTYSLSSTL | |
| | TLSKADYEKHKVYACEVTHQGLSS | |
| | PVTKSFNRGES (SEQ ID NO: | |
| | 376) | |
| Truncated | | ASTKGPSVFPLAPSSKSTSGGT |
| P241, P240A | | AALGCLVKDYFPEPVTVSWNSG |
| and P241A, | | ALTSGVHTCPAVLQSSGLYSLS |
| F174C, | | SVVTVPSSSLGTQTYICNVNHK |
| C233S IgG1 | | PSNTKVDKKVEPKSSDKTHTCA |
| F(ab′) | | A (SEQ ID NO: 377) |
| CBD1033.37 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 378) | TQTYICNVNHKPSNTKVDKKVE |
| | | PKSSDKTHTCAA (SEQ ID |
| | | NO: 379) |
| Truncated | | ASTKGPSVFPLAPSSRSTSEST |
| P241, P240A | | AALGCLVKDYFPEPVTVSWNSG |
| and P241A, | | ALTSGVHTCPAVLQSSGLYSLS |
| F174C, | | SVVTVPSSSLGTKTYTCNVDHK |
| C127S IgG4 | | PSNTKVDKRVESKYGPPCAA |
| F(ab′) | | |
| CBD1033.24 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSRSTSESTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 378) | TKTYTCNVDHKPSNTKVDKRVE |
| | | SKYGPPCAA (SEQ ID NO: |
| | | 381) |
| Truncated | | ASTKGPSVFPLAPSSKSTSGGT |
| P240, | | AALGCLVKDYFPEPVTVSWNSG |
| F174C, | | ALTSGVHTCPAVLQSSGLYSLS |
| C233S IgG1 | | SVVTVPSSSLGTQTYICNVNHK |
| F(ab′) | | PSNTKVDKKVEPKSSDKTHTCP |
| | | (SEQ ID NO: 382) |
| CBD1033.42 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 378) | TQTYICNVNHKPSNTKVDKKVE |
| | | PKSSDKTHTCP (SEQ ID |
| | | NO: 383) |
| Truncated | | ASTKGPSVFPLAPSSKSTSGGT |
| T238, F174C | | AALGCLVKDYFPEPVTVSWNSG |
| IgG1 F(ab′) | | ALTSGVHTCPAVLQSSGLYSLS |
| | | SVVTVPSSSLGTQTYICNVNHK |
| | | PSNTKVDKKVEPKSCDKTHT |
| | | (SEQ ID NO: 384) |
| CBD1033.44 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 378) | TQTYICNVNHKPSNTKVDKKVE |
| | | PKSCDKTHT (SEQ ID NO: |
| | | 385) |
| Truncated | | ASTKGPSVFPLAPSSKSTSGGT |
| P238, | | AALGCLVKDYFPEPVTVSWNSG |
| F174C, | | ALTSGVHTCPAVLQSSGLYSLS |
| C233S IgG1 | | SVVTVPSSSLGTQTYICNVNHK |
| F(ab′) | | PSNTKVDKKVEPKSSDKTHT |
| | | (SEQ ID NO: 386) |
| S162C | RTVAAPSVFIFPPSDEQLKSGTAS | |
| Kappa chain | VVCLLNNFYPREAKVQWKVDNALQ | |
| | SGNSQECVTEQDSKDSTYSLSSTL | |
| | TLSKADYEKHKVYACEVTHQGLSS | |
| | PVTKSFNRGEC (SEQ ID NO: | |
| | 387) | |
| CBD1033.45 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | EC (SEQ ID NO: 388) | TQTYICNVNHKPSNTKVDKKVE |
| | | PKSSDKTHT (SEQ ID NO: |
| | | 389) |
| Truncated | | ASTKGPSVFPLAPSSRSTSEST |
| P239, | | AALGCLVKDYFPEPVTVSWNSG |
| F174C, | | ALTSGVHTCPAVLQSSGLYSLS |
| C127S IgG4 | | SVVTVPSSSLGTKTYTCNVDHK |
| F(ab′) | | PSNTKVDKRVESKYGPPC |
| | | (SEQ ID NO: 390) |
| CBD1033.48 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSRSTSESTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 378) | TKTYTCNVDHKPSNTKVDKRVE |
| | | SKYGPPC (SEQ ID NO: |
| | | 391) |
| CBD1381.37 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYQGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 378) | TQTYICNVNHKPSNTKVDKKVE |
| | | PKSSDKTHTCAA (SEQ ID |
| | | NO: 392) |
| CBD1382.37 | DIQLTQSPSSLSASVGDRATITCR | QVQLVQSGAEVKKPGASVKVSC |
| | ASESVDGFGNSFMNWYQQKPGKAP | KASRYTFTDYNLHWVRQAPGQG |
| | KLLIYLASNLESGVPSRFSGSGSG | LEWMGFIYPYAGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 378) | TQTYICNVNHKPSNTKVDKKVE |
| | | PKSSDKTHTCAA (SEQ ID |
| | | NO: 393) |
| CBD1444.37 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYNGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 394) | TQTYICNVNHKPSNTKVDKKVE |
| | | PKSSDKTHTCAA (SEQ ID |
| | | NO: 379) |
| CBD1622.37 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYQGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 394) | TQTYICNVNHKPSNTKVDKKVE |
| | | PKSSDKTHTCAA (SEQ ID |
| | | NO: 392) |
| CBD1623.37 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYAGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 394) | TQTYICNVNHKPSNTKVDKKVE |
| | | PKSSDKTHTCAA (SEQ ID |
| | | NO: 393) |
| CBD1622.42 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYQGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 394) | TQTYICNVNHKPSNTKVDKKVE |
| | | PKSSDKTHTCP (SEQ ID |
| | | NO: 395) |
| CBD1623.42 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYAGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 394) | TQTYICNVNHKPSNTKVDKKVE |
| | | PKSSDKTHTCP (SEQ ID |
| | | NO: 396) |
| CBD1622.44 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYQGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 394) | TQTYICNVNHKPSNTKVDKKVE |
| | | PKSCDKTHT (SEQ ID NO: |
| | | 397) |
| CBD1623.44 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYAGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | ES (SEQ ID NO: 394) | TQTYICNVNHKPSNTKVDKKVE |
| | | PKSCDKTHT (SEQ ID NO: |
| | | 398) |
| CBD1622.45 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYQGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | EC (SEQ ID NO: 399) | TQTYICNVNHKPSNTKVDKKVE |
| | | PKSSDKTHT (SEQ ID NO: |
| | | 400) |
| CBD1623.45 | DIQLTQSPSSLSASVGDRATITCR | QVQLVQSGAEVKKPGASVKVSC |
| | ASESVSGFGNSFMNWYQQKPGKAP | KASRYTFTDYNLHWVRQAPGQG |
| | KLLIYLASNLESGVPSRFSGSGSG | LEWMGFIYPYAGGTGYAQKFQG |
| | TDFTLTISSVQPEDFATYYCQQNN | RVTMTVDTSTSTAYMELSSLRS |
| | EDPYTFGQGTKLEIKRTVAAPSVF | EDTAVYYCARDHRYNEGVSFDY |
| | IFPPSDEQLKSGTASVVCLLNNFY | WGQGTTVTVSSASTKGPSVFPL |
| | PREAKVQWKVDNALQSGNSQECVT | APSSKSTSGGTAALGCLVKDYF |
| | EQDSKDSTYSLSSTLTLSKADYEK | PEPVTVSWNSGALTSGVHTCPA |
| | HKVYACEVTHQGLSSPVTKSFNRG | VLQSSGLYSLSSVVTVPSSSLG |
| | EC (SEQ ID NO: 399) | TQTYICNVNHKPSNTKVDKKVE |
| | | PKSSDKTHT (SEQ ID NO: |
| | | 401) |

TABLE 19
Summary of full-length and F(ab′) humanized anti-CD8A variants constructs.

| | | VH constant | | VL constant |
|---|---|---|---|---|
| Name | VH | region | VL | region |
| CBD1032 | h1017-H1 | Fc-silenced IgG1 | h1017-L1 | Kappa chain |
| | (SEQ ID NO: 189) | (SEQ ID NO: 218) | (SEQ ID NO: 195) | (SEQ ID NO: 216) |
| CBD1033 | h1017-H2 | Fc-silenced IgG1 | h1017-L2 | Kappa chain |
| | (SEQ ID NO: 190) | (SEQ ID NO: 218) | (SEQ ID NO: 196) | (SEQ ID NO: 216) |
| CBD1034 | h1017-H2 | Fc-silenced IgG1 | h1017-L3 | Kappa chain |
| | (SEQ ID NO: 190) | (SEQ ID NO: 218) | (SEQ ID NO: 197) | (SEQ ID NO: 216) |
| CBD1035 | h1017-H3 | Fc-silenced IgG1 | h1017-L2 | Kappa chain |
| | (SEQ ID NO: 191) | (SEQ ID NO: 218) | (SEQ ID NO: 196) | (SEQ ID NO: 216) |
| CBD1036 | h1017-H3 | Fc-silenced IgG1 | h1017-L3 | Kappa chain |
| | (SEQ ID NO: 191) | (SEQ ID NO: 218) | (SEQ ID NO: 197) | (SEQ ID NO: 216) |
| CBD1037 | h1017-H4 | Fc-silenced IgG1 | h1017-L2 | Kappa chain |
| | (SEQ ID NO: 192) | (SEQ ID NO: 218) | (SEQ ID NO: 196) | (SEQ ID NO: 216) |
| CBD1038 | h1017-H4 | Fc-silenced IgG1 | h1017-L3 | Kappa chain |
| | (SEQ ID NO: 192) | (SEQ ID NO: 218) | (SEQ ID NO: 197) | (SEQ ID NO: 216) |
| CBD1039 | h1017-H5 | Fc-silenced IgG1 | h1017-L2 | Kappa chain |
| | (SEQ ID NO: 193) | (SEQ ID NO: 218) | (SEQ ID NO: 196) | (SEQ ID NO: 216) |
| CBD1040 | h1017-H5 | Fc-silenced IgG1 | h1017-L3 | Kappa chain |
| | (SEQ ID NO: 193) | (SEQ ID NO: 218) | (SEQ ID NO: 197) | (SEQ ID NO: 216) |
| CBD1041 | h1017-H6 | Fc-silenced IgG1 | h1017-L4 | Kappa chain |
| | (SEQ ID NO: 207) | (SEQ ID NO: 218) | (SEQ ID NO: 213) | (SEQ ID NO: 216) |
| CBD1042 | h1017-H7 | Fc-silenced IgG1 | h1017-L4 | Kappa chain |
| | (SEQ ID NO: 208) | (SEQ ID NO: 218) | (SEQ ID NO: 213) | (SEQ ID NO: 216) |
| CBD1043 | h1017-H7 | Fc-silenced IgG1 | h1017-L5 | Kappa chain |
| | (SEQ ID NO: 208) | (SEQ ID NO: 218) | (SEQ ID NO: 214) | (SEQ ID NO: 216) |
| CBD1044 | h1017-H7 | Fc-silenced IgG1 | h1017-L6 | Kappa chain |
| | (SEQ ID NO: 208) | (SEQ ID NO: 218) | (SEQ ID NO: 215) | (SEQ ID NO: 216) |
| CBD1045 | h1017-H8 | Fc-silenced IgG1 | h1017-L5 | Kappa chain |
| | (SEQ ID NO: 209) | (SEQ ID NO: 218) | (SEQ ID NO: 214) | (SEQ ID NO: 216) |
| CBD1046 | h1017-H8 | Fc-silenced IgG1 | h1017-L6 | Kappa chain |
| | (SEQ ID NO: 209) | (SEQ ID NO: 218) | (SEQ ID NO: 215) | (SEQ ID NO: 216) |
| CBD1047 | h1017-H9 | Fc-silenced IgG1 | h1017-L5 | Kappa chain |
| | (SEQ ID NO: 210) | (SEQ ID NO: 218) | (SEQ ID NO: 214) | (SEQ ID NO: 216) |
| CBD1048 | h1017-H9 | Fc-silenced IgG1 | h1017-L6 | Kappa chain |
| | (SEQ ID NO: 210) | (SEQ ID NO: 218) | (SEQ ID NO: 215) | (SEQ ID NO: 216) |
| CBD1049 | h1017-H10 | Fc-silenced IgG1 | h1017-L5 | Kappa chain |
| | (SEQ ID NO: 211) | (SEQ ID NO: 218) | (SEQ ID NO: 214) | (SEQ ID NO: 216) |
| CBD1050 | h1017-H10 | Fc-silenced IgG1 | h1017-L6 | Kappa chain |
| | (SEQ ID NO: 211) | (SEQ ID NO: 218) | (SEQ ID NO: 215) | (SEQ ID NO: 216) |
| CBD1128 | h1017-H1 | Fc-silenced IgG1 | h1017-L2 | Kappa chain |
| | (SEQ ID NO: 189) | (SEQ ID NO: 218) | (SEQ ID NO: 196) | (SEQ ID NO: 216) |
| CBD1129 | h1017-H2 | Fc-silenced IgG1 | h1017-L1 | Kappa chain |
| | (SEQ ID NO: 190) | (SEQ ID NO: 218) | (SEQ ID NO: 195) | (SEQ ID NO: 216) |
| CBD1380 | N55S of h1017-H2 | Fc-silenced IgG1 | h1017-L2 | Kappa chain |
| | (SEQ ID NO: 202) | (SEQ ID NO: 218) | (SEQ ID NO: 196) | (SEQ ID NO: 216) |
| CBD1381 | N55Q of h1017-H2 | Fc-silenced IgG1 | h1017-L2 | Kappa chain |
| | (SEQ ID NO: 203) | (SEQ ID NO: 218) | (SEQ ID NO: 196) | (SEQ ID NO: 216) |
| CBD1382 | N55Q of h1017-H2 | Fc-silenced IgG1 | h1017-L2 | Kappa chain |
| | (SEQ ID NO: 204) | (SEQ ID NO: 218) | (SEQ ID NO: 196) | (SEQ ID NO: 216) |
| CBD1383 | N55D of h1017-H2 | Fc-silenced IgG1 | h1017-L2 | Kappa chain |
| | (SEQ ID NO: 205) | (SEQ ID NO: 218) | (SEQ ID NO: 196) | (SEQ ID NO: 216) |
| CBD1443 | h1017-H2 | Fc-silenced IgG1 | D30E of h1017-L2 | Kappa chain |
| | (SEQ ID NO: 190) | (SEQ ID NO: 218) | (SEQ ID NO: 350) | (SEQ ID NO: 216) |
| CBD1444 | h1017-H2 | Fc-silenced IgG1 | D30S of h1017-L2 | Kappa chain |
| | (SEQ ID NO: 190) | (SEQ ID NO: 218) | (SEQ ID NO: 351) | (SEQ ID NO: 216) |
| CBD1445 | h1017-H2 | Fc-silenced IgG1 | D30A of h1017-L2 | Kappa chain |
| | (SEQ ID NO: 190) | (SEQ ID NO: 218) | (SEQ ID NO: 352) | (SEQ ID NO: 216) |
| CBD1446 | h1017-H4 | Fc-silenced IgG1 | D30E of h1017-L2 | Kappa chain |
| | (SEQ ID NO: 192) | (SEQ ID NO: 218) | (SEQ ID NO: 350) | (SEQ ID NO: 216) |
| CBD1447 | h1017-H4 | Fc-silenced IgG1 | D30S of h1017-L2 | Kappa chain |
| | (SEQ ID NO: 192) | (SEQ ID NO: 218) | (SEQ ID NO: 351) | (SEQ ID NO: 216) |
| CBD1448 | h1017-H4 | Fc-silenced IgG1 | D30A of h1017-L2 | Kappa chain |
| | (SEQ ID NO: 192) | (SEQ ID NO: 218) | (SEQ ID NO: 352) | (SEQ ID NO: 216) |
| CBD1449 | N55S of h1017-H4 | Fc-silenced IgG1 | h1017-L2 | Kappa chain |
| | (SEQ ID NO: 353) | (SEQ ID NO: 218) | (SEQ ID NO: 196) | (SEQ ID NO: 216) |
| CBD1450 | N55Q of h1017-H4 | Fc-silenced IgG1 | h1017-L2 | Kappa chain |
| | (SEQ ID NO: 354) | (SEQ ID NO: 218) | (SEQ ID NO: 196) | (SEQ ID NO: 216) |
| CBD1451 | N55A of h1017-H4 | Fc-silenced IgG1 | h1017-L2 | Kappa chain |
| | (SEQ ID NO: 355) | (SEQ ID NO: 218) | (SEQ ID NO: 196) | (SEQ ID NO: 216) |
| CBD1575 | N55Q of h1017-H4 | Fc-silenced IgG1 | D30S of h1017-L2 | Kappa chain |
| | (SEQ ID NO: 354) | (SEQ ID NO: 218) | (SEQ ID NO: 351) | (SEQ ID NO: 216) |
| CBD1576 | N55A of h1017-H4 | Fc-silenced IgG1 | D30S of h1017-L2 | Kappa chain |
| | (SEQ ID NO: 355) | (SEQ ID NO: 218) | (SEQ ID NO: 351) | (SEQ ID NO: 216) |
| CBD1622 | N55Q of h1017-H2 | Fc-silenced IgG1 | D30S of h1017-L2 | Kappa chain |
| | (SEQ ID NO: 203) | (SEQ ID NO: 218) | (SEQ ID NO: 351) | (SEQ ID NO: 216) |
| CBD1623 | N55A of h1017-H2 | Fc-silenced IgG1 | D30S of h1017-L2 | Kappa chain |
| | (SEQ ID NO: 204) | (SEQ ID NO: 218) | (SEQ ID NO: 196) | (SEQ ID NO: 216) |
| CBD1033.6 | h1017-H2 | IgG1 F(ab′) | h1017-L2 | Kappa chain |
| | (SEQ ID NO: 190) | (SEQ ID NO: 363) | (SEQ ID NO: 196) | (SEQ ID NO: 363) |
| CBD1033.61 | h1017-H2 | IgG4 F(ab′) | h1017-L2 | Kappa chain |
| | (SEQ ID NO: 190) | (SEQ ID NO: 366) | (SEQ ID NO: 196) | (SEQ ID NO: 363) |
| CBD1033.9 | h1017-H2 | Truncated P245 IgG1 F(ab′) | h1017-L2 | Kappa chain |
| | (SEQ ID NO: 190) | (SEQ ID NO: 368) | (SEQ ID NO: 196) | (SEQ ID NO: 363) |
| CBD1033.10 | h1017-H2 | Truncated P245 IgG4 F(ab′) | h1017-L2 | Kappa chain |
| | (SEQ ID NO: 190) | (SEQ ID NO: 370) | (SEQ ID NO: 196) | (SEQ ID NO: 363) |
| CBD1033.40 | h1017-H2 | Truncated P241, P240A | h1017-L2 | Kappa chain |
| | (SEQ ID NO: 190) | and P241A IgG1 F(ab′) | (SEQ ID NO: 196) | (SEQ ID NO: 363) |
| | | (SEQ ID NO: 372) | | |
| CBD1033.12 | h1017-H2 | Truncated P241, P240A | h1017-L2 | Kappa chain |
| | (SEQ ID NO: 190) | and P241A IgG4 F(ab′) | (SEQ ID NO: 196) | (SEQ ID NO: 363) |
| | | (SEQ ID NO: 374) | | |
| CBD1033.37 | h1017-H2 | Truncated P241, P240A | h1017-L2 | S162C and C214S Kappa |
| | (SEQ ID NO: 190) | and P241A, F174C, | (SEQ ID NO: 196) | chain (SEQ ID NO: 376) |
| | | C233S IgG1 F(ab′) | | |
| | | (SEQ ID NO: 377) | | |
| CBD1033.24 | h1017-H2 | Truncated P241, P240A | h1017-L2 | S162C and C214S Kappa |
| | (SEQ ID NO: 190) | and P241A, F174C, | (SEQ ID NO: 196) | chain (SEQ ID NO: 376) |
| | | C127S IgG4 F(ab′) | | |
| | | (SEQ ID NO: 380) | | |
| CBD1033.42 | h1017-H2 | Truncated P240, F174C, | h1017-L2 | S162C and C214S Kappa |
| | (SEQ ID NO: 190) | C233S IgG1 F(ab′) | (SEQ ID NO: 196) | chain (SEQ ID NO: 376) |
| | | (SEQ ID NO: 382) | | |
| CBD1033.44 | h1017-H2 | Truncated T238, F174C | h1017-L2 | S162C and C214S Kappa |
| | (SEQ ID NO: 190) | IgG1 F(ab′) | (SEQ ID NO: 196) | chain (SEQ ID NO: 376) |
| | | (SEQ ID NO: 384) | | |
| CBD1033.45 | h1017-H2 | Truncated P238, F174C, | h1017-L2 | S162C Kappa chain |
| | (SEQ ID NO: 190) | C233S IgG1 F(ab′) | (SEQ ID NO: 196) | (SEQ ID NO: 387) |
| | | (SEQ ID NO: 386) | | |
| CBD1033.48 | h1017-H2 | Truncated P239, F174C, | h1017-L2 | S162C and C214S Kappa |
| | (SEQ ID NO: 190) | C127S IgG4 F(ab′) | (SEQ ID NO: 196) | chain (SEQ ID NO: 376) |
| | | (SEQ ID NO: 390) | | |
| CBD1381.37 | N55Q of h1017-H2 | Truncated P241, P240A | h1017-L2 | S162C and C214S Kappa |
| | (SEQ ID NO: 203) | and P241A, F174C, | (SEQ ID NO: 196) | chain (SEQ ID NO: 376) |
| | | C233S IgG1 F(ab′) | | |
| | | (SEQ ID NO: 377) | | |
| CBD1382.37 | N55A of h1017-H2 | Truncated P241, P240A | h1017-L2 | S162C and C214S Kappa |
| | (SEQ ID NO: 204) | and P241A, F174C, | (SEQ ID NO: 196) | chain (SEQ ID NO: 376) |
| | | C233S IgG1 F(ab′) | | |
| | | (SEQ ID NO: 377) | | |
| CBD1444.37 | h1017-H2 | Truncated P241, P240A | D30S of h1017-L2 | S162C and C214S Kappa |
| | (SEQ ID NO: 190) | and P241A, F174C, | (SEQ ID NO: 351) | chain (SEQ ID NO: 376) |
| | | C233S IgG1 F(ab′) | | |
| | | (SEQ ID NO: 377) | | |
| CBD1622.37 | N55Q of h1017-H2 | Truncated P241, P240A | D30S of h1017-L2 | S162C and C214S Kappa |
| | (SEQ ID NO: 203) | and P241A, F174C, | (SEQ ID NO: 351) | chain (SEQ ID NO: 376) |
| | | C233S IgG1 F(ab′) | | |
| | | (SEQ ID NO: 377) | | |
| CBD1623.37 | N55A of h1017-H2 | Truncated P241, P240A | D30S of h1017-L2 | S162C and C214S Kappa |
| | (SEQ ID NO: 204) | and P241A, F174C, | (SEQ ID NO: 351) | chain (SEQ ID NO: 376) |
| | | C233S IgG1 F(ab′) | | |
| | | (SEQ ID NO: 377) | | |
| CBD1622.42 | N55Q of h1017-H2 | Truncated P240, F174C, | D30S of h1017-L2 | S162C and C214S Kappa |
| | (SEQ ID NO: 203) | C233S IgG1 F(ab′) | (SEQ ID NO: 351) | chain (SEQ ID NO: 376) |
| | | (SEQ ID NO: 382) | | |
| CBD1623.42 | N55A of h1017-H2 | Truncated P240, F174C, | D30S of h1017-L2 | S162C and C214S Kappa |
| | (SEQ ID NO: 204) | C233S IgG1 F(ab′) | (SEQ ID NO: 351) | chain (SEQ ID NO: 376) |
| | | (SEQ ID NO: 382) | | |
| CBD1622.44 | N55Q of h1017-H2 | Truncated T238, F174C | D30S of h1017-L2 | S162C and C214S Kappa |
| | (SEQ ID NO: 203) | IgG1 F(ab′) | (SEQ ID NO: 351) | chain (SEQ ID NO: 376) |
| | | (SEQ ID NO: 384) | | |
| CBD1623.44 | N55A of h1017-H2 | Truncated T238, F174C | D30S of h1017-L2 | S162C and C214S Kappa |
| | (SEQ ID NO: 204) | IgG1 F(ab′) | (SEQ ID NO: 351) | chain (SEQ ID NO: 376) |
| | | (SEQ ID NO: 384) | | |
| CBD1622.45 | N55Q of h1017-H2 | Truncated P238, F174C, | D30S of h1017-L2 | S162C Kappa chain |
| | (SEQ ID NO: 203) | C233S IgG1 F(ab′) | (SEQ ID NO: 351) | (SEQ ID NO: 387) |
| | | (SEQ ID NO: 386) | | |
| CBD1623.45 | N55A of h1017-H2 | Truncated P238, F174C, | D30S of h1017-L2 | S162C Kappa chain |
| | (SEQ ID NO: 204) | C233S IgG1 F(ab′) | (SEQ ID NO: 351) | (SEQ ID NO: 387) |
| | | (SEQ ID NO: 386) | | |

### Embodiments

Embodiment 1: A nucleic acid molecule comprising in 5′ to 3′ order, a 5′ untranslated region (UTR), an open reading frame (ORF), and a 3′ UTR wherein the 3′ UTR comprises RPS3A-PNLIP tandem 3′ UTR (SEQ ID NO: 43) or PNLIP-RPS3A tandem 3′ UTR (SEQ ID NO: 44) or a variant thereof having ≥95% sequence identity to SEQ ID NO: 43 or SEQ ID NO: 44.

Embodiment 2: A nucleic acid molecule comprising in 5′ to 3′ order, a 5′ untranslated region (UTR), an open reading frame (ORF), and a 3′ UTR wherein the 5′ UTR and 3′ UTR comprise UTR sequences, or a variant thereof having ≥95% sequence identity to the UTR sequences, selected from one of the following pairs:
a) mouse β-globin (mHBB) 5′ UTR (SEQ ID NO: 21) and pancreatic triacylglycerol lipase (PNLIP) 3′ UTR (SEQ ID NO: 41), ribosomal protein S3A (RPS3A) 3′ UTR (SEQ ID NO: 42), RPS3A-PNLIP tandem 3′ UTR (SEQ ID NO: 43), PNLIP-RPS3A tandem 3′ UTR (SEQ ID NO: 44), human hemoglobin subunit α1 (hHBA1) 3′ UTR (SEQ ID NO: 45) or human hemoglobin subunit α1 with 3 miRNA122 binding sites (hHBA1-3× miR122 bs) 3′ UTR (SEQ ID NO: 46);
b) carboxypeptidase A1 (CPA1) 5′ UTR (SEQ ID NO: 22) and CPA1 3′ UTR (SEQ ID NO: 47) wherein the ORF does not encode CPA1 or a protein having ≥60% sequence identity with CPA1;
c) defensin alpha 3 (DEFA3) 5′ UTR (SEQ ID NO: 23) and DEFA3 3′ UTR (SEQ ID NO: 48) wherein the ORF does not encode DEFA3 or a protein having ≥60% sequence identity with DEFA3;
d) human albumin (hAlb) 5′ UTR (SEQ ID NO: 24) and hHBA1 3′ UTR (SEQ ID NO: 45);
e) eIF4G aptamer ×1 (SEQ ID NO: 26) and hHBA1 3′ UTR (SEQ ID NO: 45); or
f) aptamer control (SEQ ID NO: 27) and hHBA1 3′ UTR (SEQ ID NO: 45).

Embodiment 3: A nucleic acid molecule comprising in 5′ to 3′ order, a 5′ untranslated region (UTR), an open reading frame (ORF), and a 3′ UTR wherein the 5′ UTR and 3′ UTR comprise UTR sequences, or a variant thereof having ≥95% sequence identity to the UTR sequences, selected from one of the following pairs:
a) mouse β-globin (mHBB) 5′ UTR (SEQ ID NO: 11) and pancreatic triacylglycerol lipase (PNLIP) 3′ UTR (SEQ ID NO: 31), ribosomal protein S3A (RPS3A) 3′ UTR (SEQ ID NO: 32), RPS3A-PNLIP tandem 3′ UTR (SEQ ID NO: 33), PNLIP-RPS3A tandem 3′ UTR (SEQ ID NO: 34), human hemoglobin subunit α1 (hHBA1) 3′ UTR (SEQ ID NO: 35) or human hemoglobin subunit α1 with 3 miRNA122 binding sites (hHBA1-3× miR122 bs) 3′ UTR (SEQ ID NO: 36);
b) carboxypeptidase A1 (CPA1) 5′ UTR (SEQ ID NO: 12) and CPA1 3′ UTR (SEQ ID NO: 37);
c) defensin alpha 3 (DEFA3) 5′ UTR (SEQ ID NO: 13) and DEFA3 3′ UTR (SEQ ID NO: 38);
d) human albumin (hAlb) 5′ UTR (SEQ ID NO: 14) and hHBA1 3′ UTR (SEQ ID NO: 35);
e) hemoglobin subunit alpha 1 (HBA) 5′ UTR (SEQ ID NO: 15) and amino-terminal enhancer of split and mitochondrially encoded 12S rRNA(AES-mtRNR1) 3′ UTR (SEQ ID NO: 39);
f) eIF4G aptamer ×1 (SEQ ID NO: 16) and hHBA1 3′ UTR (SEQ ID NO: 35);
g) aptamer control (SEQ ID NO: 27) and hHBA1 3′ UTR (SEQ ID NO: 45); or
h) a synthetic 5′ UTR (SEQ ID NO: 18) and hHBA1 3′ UTR (SEQ ID NO: 35).

Embodiment 4: The nucleic acid molecule of any one of embodiments 1 to 3, wherein the ORF encodes a chimeric antigen receptor (CAR).

Embodiment 5: A nucleic acid molecule comprising in 5′ to 3′ order, a 5′ untranslated region (UTR), an open reading frame (ORF) encoding a chimeric antigen receptor (CAR), and a 3′ UTR wherein the 5′ UTR and 3′ UTR comprise UTR sequences, or a variant thereof having ≥95% sequence identity to the UTR sequences, selected from one of the following pairs:
a) hemoglobin subunit alpha 1 (HBA) 5′ UTR (SEQ ID NO: 25) and amino-terminal enhancer of split and mitochondrially encoded 12S rRNA(AES-mtRNR1) 3′ UTR (SEQ ID NO: 49);
b) a synthetic 5′ UTR (SEQ ID NO: 28) and hHBA1 3′ UTR (SEQ ID NO: 45); or
c) mouse β-globin (mHBB) 5′ UTR (SEQ ID NO: 21) and human hemoglobin subunit α1 (hHBA1) 3′ UTR (SEQ ID NO: 45).

Embodiment 6: The nucleic acid molecule of any one of embodiments 1 to 5, comprising an mRNA molecule.

Embodiment 7: The nucleic acid molecule of any one of embodiments 1 to 5, comprising a DNA molecule encoding an mRNA molecule.

Embodiment 8: The mRNA of embodiment 6, wherein one or a plurality of uridine nucleosides is substituted with a modified nucleoside.

Embodiment 9: The mRNA of embodiment 8, wherein every uridine is substituted with a modified nucleoside.

Embodiment 10: The mRNA of embodiment 8 or 9, wherein the modified nucleoside is N1-methyl pseudouridine or 5-methoxyuridine or a combination thereof.

Embodiment 11: The nucleic acid molecule of any one of embodiments 1 to 10 encoding a CAR, wherein the CAR is a first, second, or third generation CAR.

Embodiment 12: The nucleic acid molecule of embodiment 11, wherein the intracellular signaling domain of the CAR is a CD3ζ signaling domain.

Embodiment 13: The nucleic acid molecule of embodiment 11 or 12 encoding a second or third generation CAR, wherein the costimulatory domain or domains are from CD28, 4-1 BB, ICOS, OX40, or CD27.

Embodiment 14: The nucleic acid molecule of any one of embodiments 11-13, wherein the transmembrane domain of the CAR is from CD8α or CD28.

Embodiment 15: The nucleic acid molecule of any one of embodiments 11-14, wherein the CAR comprises a hinge from CD28, IgG4, or CD8a.

Embodiment 16: The nucleic acid molecule of any one of embodiments 1 to 15 encoding a CAR, wherein the CAR is an anti-CD19 CAR, an anti-CD20 CAR, an anti-BCMA CAR, or an anti-fibroblast activation protein (FAP) CAR.

Embodiment 17: The nucleic acid molecule of embodiment 16, wherein the CAR comprises: a hinge, transmembrane, and co-stimulatory domain of CD28.

Embodiment 18: The nucleic acid molecule of embodiment 17, wherein the CAR comprises an anti-CD19 binding domain, CD28 hinge, transmembrane, and co-stimulatory domains, and a CD3ζ signaling domain.

Embodiment 19: The nucleic acid molecule of embodiment 18, wherein the anti-CD19 binding domain has the amino acid sequence of SEQ ID NO: 259.

Embodiment 20: The nucleic acid molecule of embodiment 16, wherein the anti-CD19 CAR has the amino acid sequence of SEQ ID NO: 4, or SEQ ID NO: 5.

Embodiment 22: The nucleic acid molecule of embodiment 20, wherein the ORF encoding the anti-CD19 CAR has a nucleotide sequence of SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, or a sequence having at least 95, 96, 97, 98, or 99% sequence identity thereto.

Embodiment 22: The nucleic acid molecule of embodiment 16, wherein the anti-CD19 CAR has an amino acid sequence of SEQ ID NO: 254, SEQ ID NO: 256, or SEQ ID NO: 258.

Embodiment 23: The nucleic acid molecule of embodiment 16, wherein the CAR comprises an anti-CD20 binding domain, IgG4 hinge, CD28 transmembrane domain, 4-1 BB costimulation domain, and a CD3ζ signaling domain.

Embodiment 24: The nucleic acid molecule of embodiment 23, wherein the anti-CD20 binding domain has the amino acid sequence of SEQ ID NO: 262.

Embodiment 25: The nucleic acid molecule of embodiment 24, wherein the anti-CD20 CAR has the amino acid sequence of SEQ ID NO: 6.

Embodiment 26: The nucleic acid molecule of embodiment 16, wherein the ORF encoding the anti-CD20 CAR has the nucleic acid sequence of SEQ ID NO: 106, or a sequence having at least 95, 96, 97, 98, or 99% sequence identity thereto.

Embodiment 27: The nucleic acid molecule of embodiment 16, wherein the CAR comprises comprising an anti-CD20 binding domain, a CD28 hinge, transmembrane domain, and costimulation domain, and a CD3ζ signaling domain.

Embodiment 28: The nucleic acid molecule of embodiment 27, wherein the anti-CD20 binding domain has the amino acid sequence of SEQ ID NO: 88.

Embodiment 29: The nucleic acid molecule of embodiment 27, wherein the anti-CD20 CAR has the amino acid sequence of SEQ ID NO: 76.

Embodiment 30: The nucleic acid molecule of embodiment 16, wherein the ORF encoding the anti-CD20 CAR has the nucleic acid sequence of SEQ ID NO: 78 or SEQ ID NO: 79, or a sequence having at least 95, 96, 97, 98, or 99% sequence identity thereto.

Embodiment 31: The nucleic acid molecule of embodiment 16, wherein the CAR comprises comprising an anti-CD20 binding domain, an IgG4 hinge, a CD28 transmembrane domain and costimulation domain, a 4-1 BB costimulation domain, and a CD3ζ signaling domain.

Embodiment 32: The nucleic acid molecule of embodiment 31, wherein the anti-CD20 binding domain has the amino acid sequence of SEQ ID NO: 87.

Embodiment 33: The nucleic acid molecule of embodiment 31, wherein the anti-CD20 CAR has the amino acid sequence of SEQ ID NO: 77.

Embodiment 34: The nucleic acid molecule of embodiment 16, wherein the ORF encoding the anti-CD20 CAR has the nucleic acid sequence of SEQ ID NO: 81 or SEQ ID NO: 82, or a sequence having at least 95, 96, 97, 98, or 99% sequence identity thereto.

Embodiment 35: The nucleic acid molecule of embodiment 16, wherein the CAR comprises an anti-FAP binding domain hinge and transmembrane from CD8, a 4-1 BB co-stimulatory domain, and a CD3ζ signaling domain.

Embodiment 36: The nucleic acid molecule of embodiment 35, wherein the anti-FAP binding domain has the nucleic acid sequence of SEQ ID NO: 303 or 305.

Embodiment 37: A nucleic acid molecule, wherein the nucleic acid molecule is an mRNA of RM_61321 (SEQ ID NO: 113), RM_61324 (SEQ ID NO: 114), RM_61326 (SEQ ID NO: 115), RM_61329 (SEQ ID NO: 116), RM_61330 (SEQ ID NO: 117), RM_61334 (SEQ ID NO: 118), RM_61340 (SEQ ID NO: 119), RM_61341 (SEQ ID NO: 120), RM_61347 (SEQ ID NO: 121), RM_61348 (SEQ ID NO: 122), RM_61349 (SEQ ID NO: 123), RM_61350 (SEQ ID NO: 124), RM_61351 (SEQ ID NO: 125), RM_61352 (SEQ ID NO: 126), RM_61353 (SEQ ID NO: 127), RM_61355 (SEQ ID NO: 128), RM_61356 (SEQ ID NO: 129), RM_61357 (SEQ ID NO: 130), RM_61358 (SEQ ID NO: 131), RM_61359 (SEQ ID NO: 132), RM_61360 (SEQ ID NO: 133), RM_61378 (SEQ ID NO: 136), RM_61379 (SEQ ID NO: 137), RM_61411 (SEQ ID NO: 138), RM_61449 (SEQ ID NO: 139), RM_61450 (SEQ ID NO: 140), RM_61451 (SEQ ID NO: 141), RM_61452 (SEQ ID NO: 142), RM_61453 (SEQ ID NO: 143), RM_61454 (SEQ ID NO: 144), RM_61455 (SEQ ID NO: 145), RM_61456 (SEQ ID NO: 146), RM_61457 (SEQ ID NO: 147), RM_61458 (SEQ ID NO: 148), RM_61459 (SEQ ID NO: 149), RM_61460 (SEQ ID NO: 150), RM_61461 (SEQ ID NO: 151), RM_61462 (SEQ ID NO: 152), RM_61463 (SEQ ID NO: 153), RM_61465 (SEQ ID NO: 155), RM_61466 (SEQ ID NO: 156), RM_61467 (SEQ ID NO: 157), RM_61468 (SEQ ID NO: 158), RM_61482 (SEQ ID NO: 159), RM_61483 (SEQ ID NO: 160), RM_61484 (SEQ ID NO: 161), RM_61485 (SEQ ID NO: 162), RM_61486 (SEQ ID NO: 163), RM_61487 (SEQ ID NO: 164), RM_61488 (SEQ ID NO: 165), RM_61489 (SEQ ID NO: 166), RM_61501 (SEQ ID NO: 167), RM_61514 (SEQ ID NO: 170), RM_61515 (SEQ ID NO: 171), RM_61519 (SEQ ID NO: 175), RM_61520 (SEQ ID NO: 176), RM_61639 (SEQ ID NO: 178), RM_61653 (SEQ ID NO: 316), RM_61654 (SEQ ID NO: 317), RM_61655 (SEQ ID NO: 318), RM_61656 (SEQ ID NO: 319), RM_61657 (SEQ ID NO: 321), RM_61658 (SEQ ID NO: 322), RM_61659 (SEQ ID NO: 323), RM_61660 (SEQ ID NO: 324), or a DNA molecule encoding said mRNA molecule.

Embodiment 38: The nucleic acid molecule of embodiment 37, wherein the mRNA is RM_61321 (SEQ ID NO: 113), RM_61324 (SEQ ID NO: 114), RM_61326 (SEQ ID NO: 115), RM_61329 (SEQ ID NO: 116), RM_61330 (SEQ ID NO: 117), RM_61340 (SEQ ID NO: 119), RM_61341 (SEQ ID NO: 120), RM_61347 (SEQ ID NO: 121), RM_61348 (SEQ ID NO: 122), RM_61349 (SEQ ID NO: 123), RM_61355 (SEQ ID NO: 128), RM_61356 (SEQ ID NO: 129), RM_61357 (SEQ ID NO: 130), RM_61378 (SEQ ID NO: 136), RM_61379 (SEQ ID NO: 137), RM_61451 (SEQ ID NO: 141), RM_61452 (SEQ ID NO: 142), RM_61453 (SEQ ID NO: 143), RM_61454 (SEQ ID NO: 144), RM_61455 (SEQ ID NO: 145), RM_61456 (SEQ ID NO: 146), RM_61458 (SEQ ID NO: 148), RM_61459 (SEQ ID NO: 149), RM_61460 (SEQ ID NO: 150), RM_61461 (SEQ ID NO: 151), RM_61462 (SEQ ID NO: 152), RM_61463 (SEQ ID NO: 153), RM_61465 (SEQ ID NO: 155), RM_61466 (SEQ ID NO: 156), RM_61467 (SEQ ID NO: 157), RM_61468 (SEQ ID NO: 158), RM_61482 (SEQ ID NO: 159), RM_61483 (SEQ ID NO: 160), RM_61484 (SEQ ID NO: 161), RM_61486 (SEQ ID NO: 163), RM_61487 (SEQ ID NO: 164), RM_61488 (SEQ ID NO: 165), RM_61489 (SEQ ID NO: 166), RM_61514 (SEQ ID NO: 170), RM_61515 (SEQ ID NO: 171), RM_61519 (SEQ ID NO: 175), RM_61520 (SEQ ID NO: 176), RM_61639 (SEQ ID NO: 178), RM_61653 (SEQ ID NO: 316), RM_61654 (SEQ ID NO: 317), RM_61655 (SEQ ID NO: 318), RM_61656 (SEQ ID NO: 319), RM_61657 (SEQ ID NO: 321), RM_61658 (SEQ ID NO: 322), RM_61659 (SEQ ID NO: 323), RM_61660 (SEQ ID NO: 324), or a DNA molecule encoding said mRNA molecule.

Embodiment 39: The nucleic acid molecule of embodiment 37, wherein in the mRNA is RM_61321 (SEQ ID NO: 113), RM_61324 (SEQ ID NO: 114), RM_61326 (SEQ ID NO: 115), RM_61330 (SEQ ID NO: 117), RM_61347 (SEQ ID NO: 121), RM_61349 (SEQ ID NO: 123), RM_61350 (SEQ ID NO: 124), RM_61378 (SEQ ID NO: 136), RM_61379 (SEQ ID NO: 137), or a DNA molecule encoding said mRNA molecule.

Embodiment 40: The nucleic acid molecule of embodiment 37, wherein the mRNA is RM_61321 (SEQ ID NO: 113), RM_61324 (SEQ ID NO: 114), RM_61326 (SEQ ID NO: 115), RM_61330 (SEQ ID NO: 117), RM_61347 (SEQ ID NO: 121), RM_61349 (SEQ ID NO: 123), RM_61350 (SEQ ID NO: 124), RM_61378 (SEQ ID NO: 136), or RM_61379 (SEQ ID NO: 137), RM_61355 (SEQ ID NO: 128), RM_61357 (SEQ ID NO: 130), RM_61461 (SEQ ID NO: 151), RM_61482 (SEQ ID NO: 159), RM_61483 (SEQ ID NO: 160), RM_61486 (SEQ ID NO: 163), RM_61487 (SEQ ID NO: 164), RM_61488 (SEQ ID NO: 165), RM_61489 (SEQ ID NO: 166), RM_61458 (SEQ ID NO: 148), RM_61455 (SEQ ID NO: 145), RM_61356 (SEQ ID NO: 129), RM_61358 (SEQ ID NO: 131), or a DNA molecule encoding said mRNA molecule.

Embodiment 41: The nucleic acid molecule of embodiment 37, wherein the mRNA is RM_61321 (SEQ ID NO: 113), RM_61324 (SEQ ID NO: 114), RM_61326 (SEQ ID NO: 115), RM_61349 (SEQ ID NO: 123), RM_61350 (SEQ ID NO: 124), RM_61357 (SEQ ID NO: 130), RM_61461 (SEQ ID NO: 151), RM_61486 (SEQ ID NO: 163), RM_61487 (SEQ ID NO: 164), RM_61488 (SEQ ID NO: 165), RM_61489 (SEQ ID NO: 166), or a DNA molecule encoding said mRNA molecule.

Embodiment 42: The nucleic acid molecule of embodiment 37, wherein the mRNA is RM_61321 (SEQ ID NO: 113), RM_61324 (SEQ ID NO: 114), RM_61326 (SEQ ID NO: 115), RM_61349 (SEQ ID NO: 123), RM_61350 (SEQ ID NO: 124), RM_61330 (SEQ ID NO: 117), RM_61379 (SEQ ID NO: 137), RM_61347 (SEQ ID NO: 121), RM_61378 (SEQ ID NO: 136), RM_61348 (SEQ ID NO: 122), RM_61329 (SEQ ID NO: 116), RM_61340 (SEQ ID NO: 119), RM_61341 (SEQ ID NO: 120), RM_61351 (SEQ ID NO: 125), RM_61353 (SEQ ID NO: 127), RM_61359 (SEQ ID NO: 132), RM_61355 (SEQ ID NO: 128), RM_61357 (SEQ ID NO: 130), RM_61461 (SEQ ID NO: 151), RM_61482 (SEQ ID NO: 159), RM_61483 (SEQ ID NO: 160), RM_61486 (SEQ ID NO: 163), RM_61487 (SEQ ID NO: 164), RM_61488 (SEQ ID NO: 165), RM_61489 (SEQ ID NO: 166), RM_61458 (SEQ ID NO: 148), RM_61455 (SEQ ID NO: 145), RM_61356 (SEQ ID NO: 146), RM_61358 (SEQ ID NO: 148), RM_61459 (SEQ ID NO: 149), RM_61484 (SEQ ID NO: 161), RM_61485 (SEQ ID NO: 162), RM_61454 (SEQ ID NO: 144), RM_61451 (SEQ ID NO: 141), RM_61453 (SEQ ID NO: 143), RM_61515 (SEQ ID NO: 171), RM_61460 (SEQ ID NO: 150), RM_61456 (SEQ ID NO: 146), RM_61411 (SEQ ID NO: 138), RM_61468 (SEQ ID NO: 158), RM_61360 (SEQ ID NO: 133), RM_61466 (SEQ ID NO: 156), RM_61467 (SEQ ID NO: 157), RM_61465 (SEQ ID NO: 155), RM_61452 (SEQ ID NO: 142), RM_61501 (SEQ ID NO: 167), RM_61457 (SEQ ID NO: 147), RM_61449 (SEQ ID NO: 139), RM_61462 (SEQ ID NO: 152), RM_61463 (SEQ ID NO: 153), RM_61450 (SEQ ID NO: 140), RM_61639 (SEQ ID NO: 178), RM_61655 (SEQ ID NO: 318), RM_61656 (SEQ ID NO: 319), RM_61657 (SEQ ID NO: 321), RM_61658 (SEQ ID NO: 322), RM_61659 (SEQ ID NO: 323), RM_61660 (SEQ ID NO: 324), or a DNA molecule encoding said mRNA molecule.

Embodiment 43: The nucleic acid molecule of embodiment 41, wherein the mRNA has an extended half-life of expression compared to base constructs RM_61118 or RM_61512 when transfected into cells.

Embodiment 44: A cell comprising the nucleic acid molecule of any one of embodiments 6 or 8-42, wherein the nucleic acid molecule is an mRNA, wherein the cell does not comprise DNA encoding the mRNA.

Embodiment 45: A cell comprising a polypeptide encoded by the mRNA of embodiment 44, wherein the cell does not comprise DNA encoding the mRNA.

Embodiment 46: A pharmaceutical composition, comprising the nucleic acid molecule of any one of embodiments 6 or 8-42, wherein the nucleic acid molecule is one or more mRNA species and the one or more mRNA species is encapsulated in one or more lipid nanoparticle (LNP) species.

Embodiment 47: The pharmaceutical composition of embodiment 46, wherein each LNP species has a lipid composition that comprises about 35 mol % to about 65 mol % an ionizable cationic lipid, about 7 mol % to about 13 mol % a phospholipid, about 0.5 mol % to about 3 mol % a PEG-lipid wherein the PEG-lipid comprises non-functionalized PEG-lipid or functionalized PEG-lipid and non-functionalized PEG-lipid, and about 27 mol % to about 50 mol % a sterol.

Embodiment 48: The pharmaceutical composition of embodiment 47, wherein the PEG-lipid comprises about 0.1 mol % to about 0.3 mol % functionalized PEG-lipid.

Embodiment 49: The pharmaceutical composition of embodiment 47 or 48, wherein the non-functionalized PEG-lipid and the functionalized PEG-lipid do not comprise the same PEG-lipid.

Embodiment 50: The pharmaceutical composition of any one of embodiments 47 to 49, wherein the functionalized PEG-lipid comprises a diacyl phosphatidyl ethanolamine and the non-functionalized PEG-lipid comprises a diacyl glycerol.

Embodiment 51: The pharmaceutical composition of embodiment 50, wherein the functionalized PEG-lipid comprises distearoyl phosphatidyl ethanolamine (DSPE).

Embodiment 52: The pharmaceutical composition of embodiment 50, wherein the non-functionalized PEG-lipid comprises distearoyl glycerol (DSG).

Embodiment 53: The pharmaceutical composition of any one of embodiments 46 to 52, wherein the PEG moiety of the PEG-lipids has a molecular weight of about 1000 to about 5000.

Embodiment 54: The pharmaceutical composition of any one of embodiments 46 to 53, wherein the PEG moiety of the functionalized PEG-lipid comprises a terminal maleimide moiety.

Embodiment 55: The pharmaceutical composition of any one of embodiments 46 to 54, further comprising a binding moiety conjugated to the functionalized PEG-lipid.

Embodiment 56: The pharmaceutical composition of any one of embodiments 46 to 55, wherein the functionalized PEG-lipid is conjugated to the binding moiety through a succinimide moiety, a hydrolyzed succinimide moiety, or a thiomaleimide moiety.

Embodiment 57: The pharmaceutical composition of embodiment 55 or 56, wherein the binding moiety comprises an antibody or antigen binding domain thereof.

Embodiment 58: The pharmaceutical composition of embodiment 57, wherein the binding moiety is a whole antibody, a minibody, an F(ab′)₂ and F(ab′), an F(ab), a diabody, a single chain Fv (scFv), or a nanobody.

Embodiment 59: The pharmaceutical composition of any one of embodiments 55 to 58, wherein the binding moiety specifically binds to an immune cell surface protein selected from CD2, CD3, CD4, CD5, CD7, CD8, CD34, CD90, or CD117.

Embodiment 60: The pharmaceutical composition of embodiment 59, wherein one or more of the LNP species contains a binding moiety which specifically binds to CD8.

Embodiment 61: The pharmaceutical composition of embodiment 60, wherein the binding moiety comprises an antibody antigen binding domain that that has at least 90% identity with the amino acid sequence of the framework regions of SEQ ID NO: 188 or 206 wherein the VH comprises a heavy chain CDR1 (VH-CDR1) comprising the amino acid sequence RYTFTDYX₁LH (SEQ ID NO: 220) wherein X₁ is N, S, Q, or A, a VH-CDR2 comprising the amino acid sequence FIYPYX₁GGTG (SEQ ID NO: 221) or FIYPYX₂GGTG (SEQ ID NO: 222) wherein X₂ is N, Q, D, S, or A, and a VH-CDR3 having the amino acid sequence DHRYX₁EGVSFDY (SEQ ID NO: 223); and a light chain variable region (VL) comprising an amino acid sequence that has at least 90% identity with the amino acid sequence of the framework regions of SEQ ID NO: 194 or 212, wherein the VL comprises a CDR1 (VL-CDR1) comprising the amino acid sequence RASESVX₃GFGX₁SFMN wherein X₃ is D, E, S, or A (SEQ ID NO: 224), VL-CDR2 comprising the amino acid sequence LASX₂LES (SEQ ID NO: 225), and a VL-CDR3 having the amino acid sequence QQX₂X₂EX₃PYT (SEQ ID NO: 226).

Embodiment 62: The pharmaceutical composition of embodiment 61, wherein the antibody antigen binding domain comprises a VL region having the amino acid sequence of SEQ ID NO: 196 and a VH region having the amino acid sequence of one of SEQ ID NOs: 190 or 202-204.

Embodiment 63: The pharmaceutical composition of embodiment 61 or 62, wherein the binding moiety is a whole antibody.

Embodiment 64: The pharmaceutical composition of embodiment 63, wherein the whole antibody comprises a silenced Fc region having the amino acid sequence of SEQ ID NO: 218 or 219.

Embodiment 65: The pharmaceutical composition of embodiment 61 or 62, wherein the binding moiety is an F(ab′).

Embodiment 66: The pharmaceutical composition of embodiment 65, wherein the F(ab′) comprises a kappa light chain constant domain having the amino acid sequence of SEQ ID NO: 216.

Embodiment 67: The pharmaceutical composition of embodiment 65 or 66, further comprising either an F(ab′) IgG1 constant region having the amino acid sequence SEQ ID NO: 363, a truncated F(ab′) IgG1 constant region having the amino acid sequence SEQ ID NO: 368, an F(ab′) IgG4 constant region having the amino acid sequence SEQ ID NO: 366, or a truncated F(ab′) IgG4 constant region having the amino acid sequence SEQ ID NO: 370.

Embodiment 68: The pharmaceutical composition of embodiment 65, wherein the F(ab′) is an engineered F(ab′).

Embodiment 69: The pharmaceutical composition of embodiment 68, wherein the engineered F(ab′) comprises a kappa light chain constant domain with a S162C substitution and either an F(ab′) IgG1 or IgG4 constant region with a F174C substitution.

Embodiment 70: The pharmaceutical composition of embodiment 68, wherein the engineered F(ab′) further comprises a kappa light chain constant domain with a C214S substitution and either an F(ab′) IgG1 constant region with a C233S substitution or an F(ab′) IgG4 constant region with a C127S substitution.

Embodiment 71: The pharmaceutical composition of embodiment 68, wherein the engineered F(ab′) comprises a kappa light chain constant domain having the amino acid sequence of SEQ ID NO: 376 or SEQ ID NO: 387.

Embodiment 72: The pharmaceutical composition of embodiment 68 or 71, wherein the engineered F(ab′) comprises an F(ab′) IgG1 constant region having the amino acid sequence of SEQ ID NO: 372, SEQ ID NO: 377, SEQ ID NO: 382, SEQ ID NO: 384, or SEQ ID NO: 386.

Embodiment 73: The pharmaceutical composition of embodiment 68 or 71, wherein the engineered F(ab′) comprises an F(ab′) IgG4 constant region having the amino acid sequence of SEQ ID NO: 374, SEQ ID NO: 380, or SEQ ID NO: 390.

Embodiment 74: The pharmaceutical composition of any of embodiment 65, 66, or 71, wherein the binding moiety comprises a heavy chain having the amino acid sequence of SEQ ID NO: 365, SEQ ID NO: 369, SEQ ID NO: 373, SEQ ID NO: 379, SEQ ID NO: 383, SEQ ID NO: 385, SEQ ID NO: 389, SEQ ID NO: 392, SEQ ID NO: 393, SEQ ID NO: 395, SEQ ID NO: 396, SEQ ID NO: 397, SEQ ID NO: 398, SEQ ID NO: 400, or SEQ ID NO: 401.

Embodiment 75: The pharmaceutical composition of any of embodiment 65, 66, or 71, wherein the binding moiety comprises a heavy chain having the amino acid sequence of SEQ ID NO: 367, SEQ ID NO: 371, SEQ ID NO: 375, SEQ ID NO: 381, or SEQ ID NO: 391.

Embodiment 76: The pharmaceutical composition of any one of embodiments 46 to 75, wherein the one or more LNP species has a lipid composition that comprises about 40 to about 60 mol % ionizable cationic lipid.

Embodiment 77: The pharmaceutical composition of embodiment 76, wherein the lipid composition comprises about 58 mol % ionizable cationic lipid.

Embodiment 78: The pharmaceutical composition of any one of embodiments 46 to 77, wherein the LNP has a lipid composition that comprises about 10 mol % phospholipid.

Embodiment 79: The pharmaceutical composition of embodiment 78, wherein the phospholipid is distearoyl phosphatidylcholine.

Embodiment 80: The pharmaceutical composition of embodiment 78, wherein the phospholipid is diarachidoyl phosphatidylcholine.

Embodiment 81: The pharmaceutical composition of any one of embodiments 47 to 80, wherein a lipid composition of the LNP comprises about 33 to about 38 mol % sterol.

Embodiment 82: The pharmaceutical composition of embodiment 81, wherein the sterol comprises cholesterol.

Embodiment 83: The pharmaceutical composition of any one of embodiments 46 to 82, wherein the PEG moiety is PEG2000 for both the functionalized and non-functionalized PEG-lipid.

Embodiment 84: The pharmaceutical composition of any one of embodiments 46 to 83, wherein a lipid composition of the one or more LNP species comprises:
a. about 40 mol % to about 62 mol % ionizable cationic lipid, about 7 mol % to about 13 mol % phospholipid, about 30 mol % to about 50 mol % sterol, about 0.5 mol % to about 3 mol % total functionalized and unfunctionalized PEG-lipid and about 0.1 mol % to 0.3 mol % functionalized PEG-lipid;
b. about 50 mol % CLCL, about 10 mol % phospholipid, about 38.5 mol % sterol, about 1.4 mol % non-functionalized PEG-lipid, and about 0.1 mol % functionalized PEG-lipid;
c. about 58 mol % CLCL, about 10 mol % phospholipid, about 30.5 mol % sterol, about 1.4 mol % non-functionalized PEG-lipid, and about 0.1 mol % functionalized PEG-lipid; or
d. about 62 mol % CLCL, about 10 mol % phospholipid, about 26.5 mol % sterol, about 1.4 mol % non-functionalized PEG-lipid, and about 0.1 mol % functionalized PEG-lipid.

Embodiment 85: The pharmaceutical composition of embodiment 84, wherein the lipid composition of the LNP is CICL1:distearoylphosphatidylcholine (DSPC):cholesterol (CHOL):unfunctionalized PEG-lipid:functionalized PEG-lipid [58:10:30.5:1.4:0.1], wherein the unfunctionalized PEG-lipid is 1,2-distearoyl-glycero-3-methoxypolyethylene glycol and the functionalized PEG-lipid is 1,2-distearoyl-glycero-3-phosphoethanolamine-N-[maleimide(polyethylene glycol)].

Embodiment 86: The pharmaceutical composition of embodiment 84 or 85 having one or more LNP species comprising a binding moiety wherein at least one binding moiety is a whole antibody that comprises a VL region having the amino acid sequence of SEQ ID NO: 196 and a VH region having the amino acid sequence of one of SEQ ID NOs: 190 or 202-204.

Embodiment 87: The pharmaceutical composition of any one of embodiments 84-86, wherein at least one of the LNP species encapsulates an mRNA comprising RM_61357 (SEQ ID NO: 130), RM_61461 (SEQ ID NO: 151), RM_61488 (SEQ ID NO: 165), and RM_61489 (SEQ ID NO: 166).

Embodiment 88: The pharmaceutical composition of embodiment 87, wherein the at least one LNP species encapsulates an mRNA comprising SEQ ID NO: 130.

Embodiment 89: The pharmaceutical composition of embodiment 87, wherein the at least one LNP species encapsulates an mRNA comprising SEQ ID NO: 151.

Embodiment 90: The pharmaceutical composition of embodiment 87, wherein the at least one LNP species encapsulates an mRNA comprising SEQ ID NO: 165.

Embodiment 91: The pharmaceutical composition of embodiment 87, wherein the at least one LNP species encapsulates an mRNA comprising SEQ ID NO: 166.

Embodiment 92: A method of treating a subject with cancer or a genetic disease or an autoimmune or fibrotic disease, comprising administering to the subject a therapeutically effective dose of the pharmaceutical composition of any one of embodiments 46 to 91, the nucleic acid molecule of any one of embodiments 6 or 8-42 wherein the nucleic acid molecule is an mRNA, or the cell of embodiment 44 or 45.

Embodiment 93: The method of embodiment 92, wherein the cancer is leukemia, multiple myeloma, diffuse large B cell lymphoma, acute myeloid leukemia, Mantle Cell lymphoma, follicular lymphoma, B acute lymphoblastic leukemia, chronic lymphocytic leukemia, myelodysplastic syndrome, sarcoma, carcinoma, breast cancer, colon cancer, ovarian cancer, lung cancer, melanoma, lymphoma, testicular cancer, hematologic cancers, myeloma, or pancreatic cancer.

Embodiment 94: The method of embodiment 93, wherein the autoimmune disease is antisynthetase syndrome (idiopathic inflammatory myopathy), myocarditis, acute idiopathic thrombocytopenic purpura, chronic idiopathic thrombocytopenic purpura, dermatomyositis, Sydenham's chorea, myasthenia gravis, systemic lupus erythematosus, fibrosing alveolitis, multiple sclerosis, rheumatic fever, polyglandular syndromes, agranulocytosis, autoimmune hemolytic anemias, bullous pemphigoid, Wegener's granulomatosis, membranous nephropathy, amyotrophic lateral sclerosis, tabes dorsalis, giant cell arteritis/polymyalgia, pernicious anemia, rapidly progressive glomerulonephritis, IgA nephropathy, polyarteritis nodosa, ankylosing spondylitis, allergic responses, insulin-resistant diabetes, psoriasis, diabetes mellitus, Addison's disease, Grave's disease, diabetes, endometriosis, celiac disease, Chron's disease, Henoch-Schonlein purpura, ulcerative colitis, Goodpasture's syndrome, thromboangitis obliterans, Sjogren's syndrome, aplastic anemia, rheumatoid arthritis, sarcoidosis, scleritis, a T cell-mediated autoimmunity or a B cell-mediated autoimmunity, a B cell-mediated autoimmune disease, immune-mediated necrotizing myopathy (IMNM), chronic inflammatory demyelinating polyneuropathy (CIDP), myeloid oligodendrocyte glycoprotein autoantibody associated disease (MOGAD), amyloid light-chain amyloidosis, neuromyelitis optica (NMO) myositis, neuromyelitis optica spectrum disorders, pemphigus vulgaris, systemic sclerosis, stiff person syndrome, lupus nephritis, membranous nephropathy, severe combined immunodeficiency, Fanconi anemia, or vasculitis.

Embodiment 95: The method of embodiment 92, wherein the autoimmune disease is antisynthetase syndrome (idiopathic inflammatory myopathy), myositis (such as anti-synthetase myositis), lupus nephritis, membranous nephropathy, systemic lupus erythematosus, neuromyelitis optica spectrum disorders, myasthenia gravis, pemphigus vulgaris, systemic sclerosis, rheumatoid arthritis, stiff person syndrome, myeloid oligodendrocyte glycoprotein autoantibody associated disease (MOGAD), amyloid light-chain amyloidosis, immune-mediated necrotizing myopathy (IMNM), multiple sclerosis, Sjögren's syndrome, IgA nephropathy, severe combined immunodeficiency, or Fanconi anemia.

Embodiment 96: The method of embodiment 92, wherein the fibrotic disease or disorder is cardiac fibrosis, arthritis, idiopathic pulmonary fibrosis, nonalcoholic steatohepatitis, or tumor-associated fibroblasts.

Embodiment 97: The method of embodiment 92, wherein the genetic disease is hemoglobinopathy, sickle cell disease or β-thalassemia.

Embodiment 98: A method of depleting CD19⁺ cells in vivo comprising administering a tLNP encapsulating an mRNA encoding a chimeric antigen receptor (CAR) comprising an anti-human CD19 scFv comprising the variable domains of monoclonal antibody 47G4, a CD28 hinge/transmembrane, a CD28 costimulatory domain, and a CD3ζ signaling domain to a mammalian subject, wherein the tLNP is targeted to CD2, CD5 or CD8, whereby CD2⁺, CD5⁺, or CD8⁺ cells, respectively, are transfected in vivo to express the CAR encoded by the mRNA.

Embodiment 99: The method of embodiment 98, whereby a greater proportion of CD19⁺ cells are depleted as compared to a subject administered a same dosage of tLNP encapsulating an otherwise identical mRNA encoding a CAR comprising an anti-human CD19 scFv comprising the variable domains of monoclonal antibody 47G4, a CD8α hinge/transmembrane domains, a CD28 costimulatory domain, and a CD3ζ signaling domain.

Embodiment 100: The method of embodiments 98 or 99, wherein the CAR comprising an anti-human CD19 scFv comprising the variable domains of monoclonal antibody 47G4, a CD28 hinge/transmembrane, a CD28 costimulatory domain, and a CD3ζ signaling domain has the amino acid sequence of SEQ ID NO: 239.

Embodiment 101: A method of transiently transfecting a cell in vivo comprising administering a pharmaceutical composition of any one of embodiments 46 to 91 or the nucleic acid molecule of any one of embodiments 6 or 8-42, wherein the nucleic acid molecule is an mRNA, to a mammalian subject.

Embodiment 102: A method of depleting B cells in vivo comprising administering to the subject a therapeutically effective dose of the pharmaceutical composition of any one of embodiments 46 to 91, wherein the one or more mRNA molecules encodes an anti-CD19 CAR, an anti-CD20 CAR, an anti-BCMA CAR, or a combination thereof.

Groupings of alternative elements or embodiments of the disclosure are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All publications, patents, and patent applications mentioned in this specification are herein incorporated by reference to the same extent as if each individual publication, patent, and patent application was specifically and individually indicated to be incorporated by reference and to the extent that they are not inconsistent with this disclosure.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

APPENDIX A

| | | Commercial Source(s) (if available) | | |
|---|---|---|---|---|
| | Antigen/ | | Supplier and | Publication |
| Antibody | Target | Product Name | Catalog Number | Information |
| 1.5.3 | CD20 | | | Gazit-Bornstein et |
| | | | | al., 2010, Invest. |
| | | | | New Drugs 28: 561- |
| | | | | 574. |
| 1C5 | CD117 | c-Kit/CD117 Antibody | ThermoFisher, | |
| | | (1C5) | #MA5-15894 | |
| 1D1C2 | IL-21Rα | IL21R Monoclonal | ThermoFisher, | |
| | | Antibody (1D1C2) | #66319-1-IG | |
| 1F5 | CD20 | Anti-CD20 [1F5] | Absolute | |
| | | | Antibody, | |
| | | | #Ab01133 | |
| 1mono2A6 | CD2 | Ellis Reinherz, Dana-Farber Cancer | | |
| (T113) | | Institute, Boston, MA | | |
| 1OLD2-4C1 | CD2 | Ellis Reinherz, Dana-Farber Cancer | | US 2022/0073639 |
| (T112) | | Institute, Boston, MA | | (SEQ ID NO. 51 & |
| | | | | 52) |
| 11B1H1B4 | CD150 | CD150 Monoclonal | Fisher Scientific, | |
| | | Antibody (11B1H1B4), | #16384654 | |
| | | Invitrogen ™ | | |
| 158-4D3 | CD45 | CD45 Monoclonal | ThermoFisher, | |
| | | Antibody (158-4D3) | #MA1-7636 | |
| 190-2F2.5 | CD45RO | CD45RO (T-Cell | ThermoFisher, | |
| | | Marker) Monoclonal | #5788-MSM12- | |
| | | Antibody (190-2F2.5) | P0 | |
| 2.1.2 | CD20 | Anti-Human MS4A1 | Creative Biolabs, | US 2007/0014720 |
| | | Recombinant Antibody | #TAB-1645CL | |
| | | (2.1.2) | | |
| 22HCLC | CD117 | Phospho-c-Kit (Tyr703) | Fisher Scientific, | |
| | | Recombinant | #PI710762 | |
| | | Polyclonal Antibody | | |
| | | (22HCLC), Invitrogen ™ | | |
| 2A9 | SSEA-3 | Anti-SSEA-3/GalGb4 | AntibodySystem, | |
| | | Antibody (2A9) | #RGK27501 | |
| 2A10D6 | FCRL5 | CD307e (FcRL5) | ThermoFisher, | |
| | | Monoclonal Antibody | #MA5-48789 | |
| 2A10H7 | FCRL5 | CD307e (FcRL5) | ThermoFisher, | |
| | | Monoclonal Antibody | #MA5-38483 | |
| 2A11D6 | CD43 | CD43 Monoclonal | ThermoFisher, | |
| | | Antibody (2A11D6) | #66224-1-IG | |
| 2B8 | CD117 | CD117 (c-Kit) | ThermoFisher, | |
| | | Monoclonal Antibody | #14-1171-82 | |
| | | (2B8), eBioscience ™ | | |
| 2B8/BM | CD117 | CD117 (c-Kit) | ThermoFisher, | |
| | | Monoclonal Antibody | #MA5-28270 | |
| | | (2B8/BM) | | |
| 2D4-C9-F1 | Sca-1 | Mouse anti-Casp3- | Biocat, #130- | |
| | | 1(138-157) (2D4-C9-F1) | 10623-20-RB | |
| 2E10 | CD34 | available from multiple sources, including: | | |
| | | Anti-CD34 [2E10] | Absolute | |
| | | | Antibody, | |
| | | | #Ab01129 | |
| | | Human Anti-CD34 | Creative Biolabs, | |
| | | Recombinant Antibody | #FAMAB- | |
| | | (clone 2E10) | 1049CQ | |
| 2ST8.5H7 | CD8β | available from multiple sources, including: | | |
| | | CD8 Beta antibody \| | Bio-Rad, | |
| | | 2ST8.5H7 | #MCA1723 | |
| | | CD8 Antibody | Novus | |
| | | (2ST8.5H7) | Biologicals, | |
| | | | #NB100-65928 | |
| 307307 | FCRL5 | CD307e (FcRL5) | ThermoFisher, | |
| | | Monoclonal Antibody | #MA5-24020 | |
| 3A1E | CD7 | CD7 Chimeric | ThermoFisher, | |
| | | Recombinant Rabbit | #MHCD0800 | |
| | | Monoclonal Antibody | | |
| | | (3A1E) | | |
| 3A1F | CD7 | Anti-CD7 [3A1F] | Absolute | |
| | | | Antibody, | |
| | | | #Ab03052 | |
| 3B5 | CD8 | available from multiple sources, including: | | |
| | | CD8 Monoclonal | ThermoFisher, | |
| | | Antibody (3B5) | #MHCD0800 | |
| | | | Aviva Systems | |
| | | | Biology, | |
| | | | #OAAI00153 | |
| 3B8-2C2 | CD48 | CD48 Monoclonal | ThermoFisher, | |
| | | Antibody (3B8-2C2) | #H00000962-M01 | |
| 3F7 | CD73 | | | Qiao et al., 2019, |
| | | | | Int. J. Mol. Sci. 20 |
| | | | | (5): 1057. |
| 3F7C6 | CD68 | CD68 monoclonal | Abnova, | |
| | | antibody, clone 3F7C6 | #MAB12263 | |
| 3F7D3 | CD68 | available from multiple sources, including: | | |
| | | CD68 monoclonal | Abnova, | |
| | | antibody, clone 3F7D3 | #MAB12260 | |
| | | Anti-CD68 antibody | Abcam, | |
| | | [3F7D3] | #ab201973 | |
| 3F8BiAb | GD2 | | | Yankelevich et al., |
| | CD3 | | | 2012, Pediatr. Blood |
| | | | | Cancer 59(7): 1198- |
| | | | | 1205. |
| 3G9-2D2 | CD205 | | | Cheong et al., 2010, |
| | | | | Blood 116 (19): |
| | | | | 3828-3838. |
| 3H420 | SSEA-3 | SSEA-3 Antibody | Santa Cruz | |
| | | (3H420) | Biotechnology, | |
| | | | #sc-73066 | |
| 3T4-8B5 | CD2 | Ellis Reinherz, Dana-Farber Cancer | | |
| (T111) | | Institute, Boston, MA | | |
| 4C5 | Stro-4 | | | Thomaidou and |
| | | | | Patsavoudi, 1993, |
| | | | | Neuroscience 53: |
| | | | | 813-827. |
| 4G5 | FAP | | | WO 2021/061708; |
| | | | | WO 2021/061778 |
| 4G7 | CD19 | | | Meeker et al., 1984, |
| | | | | Hybridoma 3: 305- |
| | | | | 320. |
| 4KB5 | CD45RA | Anti-CD45RA antibody | Abcam, #ab755 | |
| | | [4KB5] | | |
| 47G4 | CD19 | | | US 2010/0104509 |
| 51.1 | CD8 | Anti-CD8 [51.1] | Absolute | |
| | | | Antibody, | |
| | | | #Ab04357 | |
| 53H8L29 | CD117 | Phospho-c-Kit (Tyr703) | ThermoFisher, | |
| | | Recombinant Rabbit | #700135 | |
| | | Monoclonal Antibody | | |
| | | (53H8L29) | | |
| 5B4G1 | CD150 | SLAM/CD150 | ThermoFisher, | |
| | | Monoclonal Antibody | #60043-1-IG | |
| | | (5B4G1) | | |
| 5B12 | CD34 | available from multiple sources, including: | | |
| | | Anti-CD34 [5B120 | Absolute | |
| | | | Antibody, | |
| | | | #Ab00422 | |
| | | Human Anti-CD34 | Creative Biolabs, | |
| | | Recombinant Antibody | #FAMAB- | |
| | | (clone 5B12) | 1047CQ | |
| 5D7 | CD5 | CD5 Monoclonal | ThermoFisher, | |
| | | Antibody (CD5-5D7), | #MHCD0505 | |
| | | APC | | |
| 5E3E1 | IL-15Rα | IL-15RA Monoclonal | ThermoFisher, | |
| | | Antibody (5E3E1) | #MHCD0505 | |
| 5E10 | CD90 | available from multiple sources, including: | | |
| | | CD90 (Thy-1) | ThermoFisher, | |
| | | Monoclonal Antibody | #14-0909-82 | |
| | | (eBio5E10 (5E10)), | | |
| | | eBioscience ™ | | |
| | | Anti-Human CD90 | Stemcell | |
| | | Antibody, Clone 5E10 | Technologies, | |
| | | | #60045 | |
| 509F6 | FCRL5 | CD307e (FcRL5) | ThermoFisher, | |
| | | Monoclonal Antibody | #50-3078-42 | |
| | | (509F6), eFluor ™ 660, | | |
| | | eBioscience ™ | | |
| 6D276 | CD29 | Anti-CD49b, CD29 | VWR, | |
| | CD49b | Complex Mouse | #USBIC2404- | |
| | | Monoclonal Antibody | 07W | |
| | | [clone: 6D276] | | |
| 6D9 | GPRC5D | Anti-GPRC5D (6D9) | Creative Biolabs, | |
| | | h(41BB-CD3ζ) CAR, | #CAR-LY1362 | |
| | | pCDCAR1 | | |
| 6F2 | CD117 | KIT Monoclonal | ThermoFisher, | |
| | | Antibody (6F2) | #H00003815-M02 | |
| 7D11 | FCRL5 | Anti-FcRH5 (clone | Creative Biolabs, | |
| | | 7D11)-SMCC-DM1 | #ADC-161CL | |
| | | ADC | | |
| 7E1 | CD48 | CD48 Recombinant | ThermoFisher, | |
| | | Rabbit Monoclonal | #MA5-38301 | |
| | | Antibody (7E1) | | |
| 8A7 | SSEA-3 | Anti-SSEA-3/GalGb4 | AntibodySystem, | |
| | | Antibody (8A7) | #RGK27504 | |
| 8D7 | CD117 | c-Kit Monoclonal | ThermoFisher, | |
| | | Antibody (8D7) | #MA5-15327 | |
| 9.1 | CD2 | Peter Linsley, Benaroya Research | | Connelly et al., |
| | | Institute, Seattle, WA | | 1998, Int. Immunol., |
| | | | | 10 (12): 1863-1872. |
| 9.6 | CD2 | Peter Linsley, Benaroya Research | | Connelly et al., |
| | | Institute, Seattle, WA | | 1998, Int. Immunol., |
| | | | | 10 (12): 1863-1872. |
| 9C5 | CD34 | | | U.S. Pat. No. 8,399,249 |
| 9D1 | CD150 | CD150 Monoclonal | ThermoFisher, | |
| | | Antibody (9D1), PE, | #12-1501-82 | |
| | | eBioscience ™ | | |
| 10A8 | FCRL5 | Anti-FcRL5 (10A8)- | Creative Biolabs, | |
| | | SPDB-DM4 ADC | #ADC-117LCT | |
| 12G10 | CD29 | available from multiple sources, including: | | |
| | | CD29 (Stem Cell | ThermoFisher, | |
| | | Marker) Monoclonal | #3688-MSM2-P0 | |
| | | Antibody (12G10) | | |
| | | CD29 antibody \| 12G10 | Bio-Rad, | |
| | | | #MCA2028 | |
| 13G9 | FCRL5 | Mouse Anti-FCRL5 | Creative Biolabs, | |
| | | Recombinant Antibody | #MOB-057CQ | |
| 17D8 | CD8 | CD8 Monoclonal | ThermoFisher, | |
| | | Antibody (17D8) | #MA1-12027 | |
| 18A5 | IL-21Rα | Human Anti-IL21R | Creative Biolabs, | |
| | | Recombinant Antibody | #HPAB-0668-CN | |
| 84-3C1 | CD43 | CD43 Monoclonal | ThermoFisher, | |
| | | Antibody (PE, | #12-0439-42 | |
| | | eBioscience ™) | | |
| 104D2 | CD117 | available from multiple sources, including: | | |
| | | Purified anti-human | BioLegend, | |
| | | CD117 (c-kit) Antibody | #313201 | |
| | | Anti-Human CD117 (c- | Stemcell Tech- | |
| | | Kit) Antibody, Clone | nologies, #60087 | |
| | | 104D2 | | |
| | | MOUSE ANTI HUMAN | Bio-Rad, | |
| | | CD117 | #MCA1841 | |
| | | c-Kit Monoclonal | ThermoFisher, | |
| | | Antibody (104D2) | #MA1-10072 | |
| 131-I- | CD45 | | | Seropian et al., |
| apamistamab | | | | 2023, Blood 142 |
| | | | | (Supplement 1): |
| | | | | 2159. |
| 131-I- | B7H3 | | | Kramer et al., 2022, |
| omburtamab | | | | J. Hematol. Oncol. |
| (131-I-8H9) | | | | 15: 165. |
| 177-Lu-DTPA- | B7H3 | | | Khatua et al., 2021, |
| omburtamab | | | | Ann. Oncol. 32 |
| | | | | (Suppl. 5): S528- |
| | | | | S529. |
| 156-4H9 | CD48 | CD48 Monoclonal | ThermoFisher, | |
| | | Antibody (eBio 156-4H9 | #14-0489-82 | |
| | | (156-4H9)), | | |
| | | eBioscience ™ | | |
| 293C3 | CD133 | available from multiple sources, including: | | |
| | | Anti-CD133 antibody | Abcam, | |
| | | [293C3] | #ab252553 | |
| | | CD133/2 Antibody, | Miltenyi Biotec, | |
| | | anti-human, pure | #130-090-851 | |
| 341 | CD48 | CD48 Recombinant | ThermoFisher, | |
| | | Rabbit Monoclonal | #MA5-29675 | |
| | | Antibody (341) | | |
| 514H12 | CD68 | CD68 Monoclonal | ThermoFisher, | |
| | | Antibody (514H12) | #MA1-80133 | |
| 1279B | SUSD2 | SUSD2 Antibody | Novus | |
| | | (1279B) | Biologicals, | |
| | | | #MAB90564 | |
| 2639B | IL-15Rα | Human IL-15R alpha | R&D Systems, | |
| | | Antibody | #MAB10900 | |
| A7R34 | CD127 | CD127 Monoclonal | ThermoFisher, | |
| | | Antibody (A7R34), | #14-1271-82 | |
| | | eBioscience ™ | | |
| A12 | CD150 | CD150 Monoclonal | ThermoFisher, | |
| (7D4) | | Antibody (A12 (7D4)), | #12-1509-42 | |
| | | PE, eBioscience ™ | | |
| AB75 | CD2 | available from multiple sources, including: | | |
| | | CD2 Monoclonal | ThermoFisher, | |
| | | Antibody (AB75) | #MA5-11373 | |
| | | Anti-CD2 Mouse | Avantor (VWR), | |
| | | Monoclonal Antibody | #GTX75367 | |
| | | [clone: AB75] | (89365-772) | |
| Ab85 | CD117 | | | WO 2019/084053 |
| ABL501 | LAG-3 | | | Park et al., 2021, |
| | PD-L1 | | | Cancer Res. 81 |
| | | | | (13_Supplement): |
| | | | | 1633. |
| ABM53F5 | CD68 | Monoclonal antibody to | Abeomics, #10- | |
| | | CD68 (Clone: | 4171 | |
| | | ABM53F5) | | |
| ABX-MA1 | CD146 | Anti-CD146 [ABX-MA1 | Absolute | |
| | | (3.19.1)] | Antibody, | |
| | | | #Ab02820 | |
| AC133 | CD133 | CD133/1 Antibody, | Miltenyi Biotec, | |
| | | anti-human, pure | #130-090-422 | |
| acazicolcept | CD28 | Acazicolcept | MedChemExpress, | |
| | | | #HY-P99420 | |
| ACK2 | CD117 | CD117 (c-Kit) | ThermoFisher, | |
| | | Monoclonal Antibody | #17-1172-82 | |
| | | (ACK2), APC, | | |
| | | eBioscience ™ | | |
| ACK4 | CD117 | c-Kit Monoclonal | ThermoFisher, | |
| | | Antibody (ACK4) | #MA1-70079 | |
| Adcitmer ® | CD56 | | | Esnault et al., 2022, |
| | | | | Br. J. Dermatol. 186 |
| | | | | (2): 295-306. |
| ADG106 | CD137 | | | Ma et al., 2024, Cell |
| | | | | Rep. Med. 5 (2): |
| | | | | 101414. |
| ADG116 | CTLA-4 | | | Park et al., 2022, J. |
| | | | | Immunother. Cancer |
| | | | | 10 (Suppl 2): A1- |
| | | | | A1603. |
| ADG126 | CTLA-4 | | | Richardson et al., |
| | | | | 2022, Ann. Oncol. |
| | | | | 33 (suppl 7): S882- |
| | | | | S883. |
| ADU-1604 | CTLA-4 | | | De Velasco Oria de |
| | | | | Rueda et al., 2023, |
| | | | | J. Immunother. |
| | | | | Cancer 11 (Suppl |
| | | | | 1): A1-A1731. |
| AFM13 | CD16a | Anti-FcgR3a/CD16a | Selleckchem, | |
| | | (AFM13) | #A2750 | |
| AGEN1181 | CTLA-4 | | | El-Khoueiry, et al., |
| | | | | 2021, J. |
| | | | | Immunother. Cancer |
| | | | | 9 (Suppl 2): A1- |
| | | | | A1054. |
| AK112 | PD-1 | Research Grade | AntibodySystem, | |
| | VEGF | Ivonescimab | #DHD12605 | |
| AK119 | CD73 | Research Grade | Cell Sciences, | |
| | | Dresbuxelimab | #DHD46603A | |
| AK129 | LAG-3 | | | Huang et al., 2022, |
| | | | | Cancer Res. 82 |
| | | | | (12_Supplement): |
| | | | | 5520. |
| alefacept | CD2 | Alefacept | MedChemExpress, | |
| | | | #HY-P99429 | |
| AlexaFluor647 | IgG | available from multiple sources, including: | | |
| anti-hIgG Fc | | | | |
| detection | | Alexa Fluor ® 647 | Jackson | |
| antibody | | AffiniPure ™ Goat Anti- | ImmunoResearch | |
| | | Human IgG, Fcγ | Laboratories, | |
| | | fragment specific | #109-605-098 | |
| | | Human IgG Alexa | R&D Systems, | |
| | | Fluor ® 647-conjugated | #FAB110R | |
| | | Antibody | | |
| alirocumab | PCSK9 | available from multiple sources, including: | | |
| | | Alirocumab | MedChemExpress, | |
| | | | #HY-P9928 | |
| | | Alirocumab Biosimilar - | Proteogenix, | |
| | | Anti-PCSK9 mAb | #PX-TA1287 | |
| | | Alirocumab (anti- | SelleckChem, | |
| | | PCSK9) | #A2047 | |
| alnuctumab | BCMA | | | U.S. Pat. No. 10,683,369 |
| AMG-172 | CD70 | | | Massard et al., |
| | | | | 2019, Cancer |
| | | | | Chemother. |
| | | | | Pharmacol. |
| | | | | 83(6): 1057-63. |
| AMG 224 | BCMA | | | U.S. Pat. No. 9,243,058 |
| AMG 228 | GITR | | | Tran et al., 2017, J. |
| | | | | Clin. Oncol. 35 |
| | | | | (15_suppl): 2521. |
| AMG 404 | PD-1 | Zeluvalimab | MedChemExpress, | |
| | | | #HY-P99957 | |
| AMM22070N | Sca-1 | Caspase-3 Mouse mAb | Leading Biology, | |
| | | | #AMM22070N | |
| anti-BCMA CARs | BCMA | | | US 2020/0246381; |
| | | | | US 2020/0339699 |
| anti-CD3 FITC | CD3 | BD Pharmingen ™ | BD Biosciences, | |
| antibody | | FITC mouse anti- | #556611 | |
| conjugate | | human CD3ε | | |
| anti-CD3 SP34-2 | CD3 | BD Pharmingen ™ | BD Biosciences, | |
| Alexa Fluor 700 | | Alexa Fluor ® 700 | #557917 | |
| conjugate | | mouse anti-human | | |
| antibody | | CD3 | | |
| anti-CD3 V450 | CD3 | available from multiple sources, including: | | |
| | | BD Horizon ™ V450 | BD Biosciences, | |
| | | Mouse Anti-Human | #560366 | |
| | | CD3 | | |
| | | violetFluor ™ 450 Anti- | Cytek, #75-0038- | |
| | | Human CD3 (UCHT1) | T025 | |
| anti-CD4 | CD4 | available from multiple sources, including: | | |
| (PerCP-Cy5.5) | | BD Pharmingen ™ | BD Biosciences, | |
| | | PerCP-Cy ™5.5 Mouse | #560650 | |
| | | Anti-Human CD4 | | |
| | | CD4 Monoclonal | ThermoFisher, | |
| | | Antibody (RM4-5), | #45-0042-82 | |
| | | PerCP-Cyanine5.5, | | |
| | | eBioscience ™ | | |
| anti-CD4 BV 421 | CD4 | Brilliant Violet 421 ™ | BioLegend, | |
| antibody | | anti-human CD4 | #317434 | |
| conjugate | | Antibody | | |
| anti-CD4 OKT4 | CD4 | Brilliant Violet 650 ™ | BioLegend, | |
| BV650 | | anti-human CD4 | #317436 | |
| conjugate | | Antibody | | |
| antibody | | | | |
| anti-CD8a | CD8α | BD Pharmingen ™ | BD Biosciences, | |
| (APC-H7) | | APC-H7 Mouse anti- | #560179 | |
| | | Human CD8 | | |
| anti-claudin 18.2 | CLDN18.2 | | | WO 2013/167259, |
| antibodies | | | | WO 2021/032157, |
| | | | | WO 2021/254481, |
| | | | | WO 2022/007808, |
| | | | | WO 2021/008463, |
| | | | | WO 2022/111616, |
| | | | | WO 2018/006882, |
| | | | | WO 2020/147321, |
| | | | | WO 2019/219089, |
| | | | | US 2020/0040101, |
| | | | | WO 2020/025792, |
| | | | | WO 2020/139956, |
| | | | | WO 2020/135201, |
| | | | | US 2024/0228610, |
| | | | | WO 2021/218874, |
| | | | | WO 2021/027850, |
| | | | | WO 2021/129765, |
| | | | | WO 2022/068854, |
| | | | | WO 2021/111003 |
| anti-FAP CARs | FAP | | | WO 2021/061778 |
| anti-FCRL5 CARS | FCRL5 | | | WO 2016/090337, |
| and ADCs | | | | WO 2017/096120, |
| | | | | WO 2022/263855, |
| | | | | WO 2024/047558, |
| | | | | Elkins et al., Mol. |
| | | | | Cancer Ther. |
| | | | | 11(10): 2222-2232 |
| | | | | (2012) |
| anti-G4S PE | G4S | G4S Linker (E7O2V) | Cell Signaling | |
| antibody | | Rabbit mAb (PE | Technology, | |
| | | Conjugate) | #38907 | |
| anti-human Fc | Fc | Brilliant Violet 421 ™ | BioLegend, | |
| secondary | | anti-human IgG Fc | #410704 | |
| antibody | | Antibody | | |
| (conjugated to | | | | |
| BV421 | | | | |
| fluorophore) | | | | |
| anti-scFv linker | scFv | MonoRab ™ Rabbit | GenScript, | |
| PE antibody | linker | Anti-scFv Cocktail [PE] | #A02285 | |
| anti-siglec-15 | Siglec-15 | | | U.S. Pat. No. 8,575,531 |
| antibody | | | | |
| anti-Whitlow | | Whitlow/218 Linker | Cell Signaling | |
| linker PE | | (E3U7Q) Rabbit mAb | Technology, | |
| antibody | | (PE Conjugate) | #62405 | |
| ANV419 | CD122 | | | Joerger et al., 2023, |
| | | | | J. Immunother. |
| | | | | Cancer 11 (11): |
| | | | | e007784. |
| ARC0672 | CD127 | CD127 Recombinant | ThermoFisher, | |
| | | Rabbit Monoclonal | #MA5-35152 | |
| | | Antibody (ARC0672) | | |
| ARX305 | CD70 | | | Skidmore et al., |
| | | | | 2023, Ann. Oncol. |
| | | | | 34 (Suppl. 2): S208. |
| ASD141 | CD11b | | | Chang et al., 2021, |
| | | | | J. Immunother. |
| | | | | Cancer 9 (Suppl. 2): |
| | | | | A1-A1054. |
| ASKB589 | CLDN18.2 | | | Zhang et al., 2023, |
| | | | | J. Clin. Oncol. 41 (4 |
| | | | | suppl.), 397. |
| ASP1951 | GITR | | | Seidel-Dugan et al., |
| | | | | 2018, J. |
| | | | | Immunother. Cancer |
| | | | | 6 (115). |
| AT002 | CD166 | available from multiple sources, including: | | |
| | | Recombinant Anti- | Antibodies.com, | |
| | | CD166 antibody | #A323924 | |
| | | Research Grade Anti- | AntibodySystem, | |
| | | Human CD166/ALCAM | #DHG64002 | |
| atibuclimab | CD14 | available from multiple sources, including: | | |
| | | Atibuclimab | MedChemExpress, | |
| | | | #HY-P99008 | |
| | | Atibuclimab (Anti- | SelleckChem, | |
| | | CD14) | #A2566 | |
| ATL301 | GD2 | Research Grade Anti- | AntibodySystem, | |
| | | Ganglioside GD2 | #DHK07801 | |
| | | (ATL301) | | |
| ATOR-1015 | OX40 | | | Veitonmaki et al., |
| | | | | 2018, Cancer Res. 78 |
| | | | | (13_Supplement): |
| | | | | 3623. |
| ATOR-1144 | GITR | | | Fritzell et al., 2019, |
| | | | | Cancer Res. 79 |
| | | | | (13_Supplement): |
| | | | | 4077. |
| axicabtagene | CD19 | | | Neelapu et al., |
| ciloleucel | | | | 2017, N. Engl. J. |
| | | | | Med. 377: 2531-44. |
| AZD0901 | CLDN18.2 | | | Raufi et al., 2024, J. |
| | | | | Clin. Oncol. 42 (16 |
| | | | | Suppl.), TPS3163. |
| AZD7789 | TIM-3 | | | Clancy-Thompson |
| | | | | et al., J. |
| | | | | Immunother. Cancer |
| | | | | 10 (Suppl. 2): A1- |
| | | | | A1603. |
| AZN-L50 | CD166 | available from multiple sources, including: | | |
| | | CD166, Human, mAB | Hycult Biotech, | |
| | | AZN-L50 | #HM2002 | |
| | | CD166 antibody [AZN- | GeneTex, | |
| | | L50] | #GTX18159 | |
| B4 | CD19 | | | Freedman et al., |
| | | | | 1987, Blood 70: |
| | | | | 418-427. |
| B4 HB12b | CD19 | | | Kansas & Tedder, |
| | | | | 1991, J. Immunol. |
| | | | | 147: 4094-4102; |
| | | | | Yazawa et al., 2005, |
| | | | | PNAS 102: 15178- |
| | | | | 15183; |
| | | | | Herbst et al., 2010, |
| | | | | J. Pharmacol. Exp. |
| | | | | Ther. 335: 213-222. |
| B7V3V2 | CD200 | Anti-Human CD200 | AntibodySystem, | |
| | | Antibody (B7V3V2) | #FHE30310 | |
| B43 | CD19 | | | Bejcek et al., 1995, |
| | | | | Cancer Res. 55 |
| | | | | (11): 2346-2351. |
| balstilimab | PD-1 | | | Moore et al., 2018, |
| | | | | J. Clin. Oncol. 36 |
| | | | | (15 suppl.): 3086. |
| barzolvolimab | CD117 | CDX 0159 | MedChemExpress, | |
| | | | #HY-P99462 | |
| basiliximab | CD25 | Basiliximab | ThermoFisher, | Onrust et al., 1999, |
| (STI-003) | | Recombinant Human | #MA5-41768 | Drugs 57, 207-213. |
| | | Monoclonal Antibody | | |
| BAT6026 | OX40 | | | Liang et al., 2023, |
| | | | | Front. Oncol., 13: |
| | | | | 1211759. |
| bavunalimab | LAG-3 | available from multiple sources, including: | | |
| | | Bavunalimab | MedChemExpress, | |
| | | | #HY-P99354 | |
| | | Bavunalimab | ChemScene, | |
| | | | #CS-0621488 | |
| BB2121 | BCMA | | | WO 2012/163805 |
| BC8-B10 | CD45 | | | Li et al., 2018, PLoS |
| | | | | One 13(10): |
| | | | | e0205135. |
| BCD-145 | CTLA-4 | available from multiple sources, including: | | |
| | | Nurulimab | MedChemExpress, | |
| | | | #HY-P99760 | |
| | | Nurulimab | AbMole, | |
| | | | #M24967 | |
| BCD-245 | GD2 | | | Clinical Trial ID: |
| | | | | NCT05782959 |
| belantamab | BCMA | | | U.S. pat. No. 9,273,141 |
| BGB-A445 | OX40 | | | Jiang et al., 2023, |
| | | | | Front. Med. 17(6): |
| | | | | 1170-1185. |
| bleselumab | CD40 | available from multiple sources, including: | | |
| | | Bleselumab | MedChemExpress, | |
| | | | #HY-P99257 | |
| | | Bleselumab | SelleckChem, | |
| | | | #A2634 | |
| BL-178-12C7 | CD45 | Anti-CD45 antibody | Abcam, | |
| | | | #ab243869 | |
| BLY3 | CD19 | | | Bejcek et al., 1995, |
| | | | | Cancer Res. 55 |
| | | | | (11): 2346-2351. |
| BMS-986156 | GITR | | | Siu et al., 2017, J. |
| | | | | Clin. Oncol. 35 |
| | | | | (15_suppl): 104. |
| BMS-986178 | OX40 | | | Gutierrez et al., |
| | | | | 2021, Clin. Cancer |
| | | | | Res. 27 (2): 460- |
| | | | | 472. |
| BMS-986179 | CD73 | | | Siu et al., 2018, |
| | | | | Cancer Res. |
| | | | | 78(13_Suppl): |
| | | | | CT180. |
| BMS-986218 | CTLA-4 | | | Engelhardt et al., |
| | | | | 2020, Cancer Res. 80 |
| | | | | (16_Supplement): |
| | | | | 4552. |
| BMS-986249 | CTLA-4 | | | Gutierrez et al., |
| | | | | 2020, J. Clin. Oncol. |
| | | | | 38 (15_suppl): 3058. |
| BMS-986258 | TIM-3 | | | Clinical Trial ID: |
| | | | | NCT03446040 |
| BMS-986393 | GPRC5D | | | Bal et al. (2023). |
| | | | | Blood 142 (Suppl. |
| | | | | 1): 219. |
| bococizumab | PCSK9 | available from multiple sources, including: | | |
| | | Bococizumab | MedChemExpress, | |
| | | | #HY-P99187 | |
| | | Bococizumab (Anti- | SelleckChem, | |
| | | PCSK9) | #A2788 | |
| | | Bococizumab | ThermoFisher, | |
| | | Recombinant Human | #MA5-42019 | |
| | | Monoclonal Antibody | | |
| bococizumab NEI | PCSK9 | | | Dyson et al. (2020). |
| | | | | MAbs 12: 1829335. |
| botensilimab | CTLA-4 | | | Wilky et al., 2022, J. |
| | | | | Immunother. |
| | | | | Cancer, 10 (Suppl. |
| | | | | 2): A1-A1603. |
| BP-02489 | CD10 | Accession No. NITE BP-02489 (JP) | | WO 2018/235247 |
| brexucabtagene | CD19 | | | Anderson et al., |
| autoleucel | | | | 2022, Ann. |
| | | | | Pharmacother. |
| | | | | 56(5): 609-619. |
| briquilimab | CD117 | available from multiple sources, including: | | |
| (JSP-191) | | Briquilimab | MedChemExpress, | |
| | | | #HY-P99488 | |
| | | Research Grade | Cell Sciences, | |
| | | Briquilimab | #DHC83102A | |
| BTI-322 | CD2 | Anti-Human CD2 | Creative Biolabs, | |
| | | Recombinant Antibody | #TAB-110CL | |
| | | (BTI-322) | | |
| BU12 | CD19 | | | Callard et al., 1992, |
| | | | | J. Immunology, |
| | | | | 148(10): 2983-2987 |
| Bu88 | CD8 | Anti-CD8 alpha | Abcam, | |
| | | Antibody | #ab316355 | |
| budigalimab | PD-1 | Budigalimab | MedChemExpress, | |
| | | | #HY-P99489 | |
| C2e10 | CD34 | available from multiple sources, including: | | |
| | | Mouse Anti-CD34 | Creative Biolabs, | |
| | | Recombinant Antibody | #HPAB-1044-FY- | |
| | | (clone C2e10); scFv | F(E) | |
| | | Fragment | | |
| | | Anti-CD34 [2E10], | Absolute | |
| | | Human IgG1, Kappa | Antibody, | |
| | | | #Ab01129 | |
| C5B12 | CD34 | available from multiple sources, including: | | |
| | | Human Anti-CD34 | Creative Biolabs, | |
| | | Recombinant Antibody | #HPAB-1043-FY- | |
| | | (clone C5B12); Fab | S(P) | |
| | | Fragment | | |
| | | Anti-CD34 [5B12], | Absolute | |
| | | Human IgG1, Kappa | Antibody, | |
| | | | #Ab00422 | |
| C8/144B | CD8α | available from multiple sources, including: | | |
| | | CD8 alpha Monoclonal | ThermoFisher, | |
| | | Antibody (C8/144B) | #MA5-13473 | |
| | | Anti-CD8 alpha | Abcam, | |
| | | antibody [C8/144B] | #ab17147 | |
| C11D5.3 | BCMA | | | WO 2010/104949 |
| C12A3.2 | | | | |
| C363.16A | CD45RB | CD45RB Monoclonal | ThermoFisher, | |
| | | Antibody (PE, | #12-0455-82 | |
| | | eBioscience ™) | | |
| cadonilimab | PD-1 | | | Pang et al., 2023, |
| | | | | MAbs 15(1): |
| | | | | 2180794. |
| camidanlumab | CD25 | | | Puzanov et al., |
| tesirine | | | | 2020, Ann. Oncol. |
| | | | | 31(suppl. 4), S710- |
| | | | | S711. |
| camrelizumab | PD-1 | Camrelizumab (anti- | SelleckChem, | |
| | | PD-1) | #A2016 | |
| carotuximab | CD105 | available from multiple sources, including: | | |
| | | Carotuximab | MedChemExpress, | |
| | | | #HY-P99494 | |
| | | Carotuximab (Anti- | SelleckChem, | |
| | | Endoglin/CD105) | #A2514 | |
| CBXS-1620 | SUSD2 | Rabbit Anti-SUSD2 | Creative Biolabs, | |
| | | Recombinant Antibody | #CBMAB-S4469- | |
| | | (CBXS-1620) | CQ | |
| CBXS-1671 | SUSD2 | Rabbit Anti-SUSD2 | Creative Biolabs, | |
| | | Recombinant Antibody | #CBMAB-S0094- | |
| | | (CBXS-1671) | CQ | |
| CBXS-1989 | SUSD2 | Mouse Anti-SUSD2 | Creative Biolabs, | |
| | | Recombinant Antibody | #CBMAB-S4758- | |
| | | (CBXS-1989) | CQ | |
| CBXS-1990 | SUSD2 | Mouse Anti-SUSD2 | Creative Biolabs, | |
| | | Recombinant Antibody | #CBMAB-S0156- | |
| | | (CBXS-1990) | CQ | |
| CBXS-3571 | SUSD2 | Human Anti-SUSD2 | Creative Biolabs, | |
| | | Recombinant Antibody | #CBMAB-S0386- | |
| | | (CBXS-3571) | CQ | |
| CBXS-3676 | SUSD2 | Rabbit Anti-SUSD2 | Creative Biolabs, | |
| | | Recombinant Antibody | #CBMAB-S0214- | |
| | | (CBXS-3676) | CQ | |
| CBYY-I0413 | IL-12R | Mouse Anti-IL12RB2 | Creative Biolabs, | |
| | | Recombinant Antibody | #CBMAB-I1506- | |
| | | (CBYY-I0683) | YY | |
| C-CAR066 Leu16 | CD20 | | | Liang et al., 2021, J. |
| and 2.1.2 | | | | Clin. Oncol. 39(15) |
| | | | | suppl: 2508) |
| CD58 (LFA-3) | CD2 | Recombinant Human | R&D Systems, | |
| | | CD58/LFA-3 His-tag | #1689-CD | |
| | | Protein | | |
| CDLA68-1 | CD68 | CD68 Antibody [clone | NSJ Bioreagents, | |
| | | CDLA68-1] | #V7045 | |
| CDX-0158 | CD117 | Anti-SCFR/c-Kit/ | BiOrbyt, | |
| | | CD117 Reference | #orb1817461 | |
| | | Antibody (CDX-0158) | | |
| cedelizumab | CD4 | available from multiple sources, including: | | |
| | | Cedelizumab | MedChemExpress, | |
| | | | #HY-P99495 | |
| | | Cedelizumab (CD4) | ProSci, #10-093 | |
| cemiplimab | PD-1 | | | Burova et al., 2017, |
| | | | | Mol. Cancer Ther. |
| | | | | 16 (5), 861-870. |
| cetrelimab | PD-1 | Cetrelimab | MedChemExpress, | |
| | | | #HY-P99499 | |
| cetuximab | EGFR | available from multiple sources, including: | | |
| | | EGF R antibody \| C225 | BioRad, | |
| | | | #MCA6102 | |
| | | Cetuximab | MedChemExpress, | |
| | | | #HY-P9905 | |
| | | Cetuximab (anti-EGFR) | SelleckChem, | |
| | | | #A2000 | |
| cevostamab | CD3 | Cevostamab | MedChemExpress, | |
| | | | #HY-P99601 | |
| ch28/11 | SSEA-4 | Chimeric | Creative Biolabs, | |
| | | (Mouse/Human) Anti- | #PABJ-0121 | |
| | | SSEA-4 Recombinant | | |
| | | Antibody (clone | | |
| | | ch28/11) | | |
| cifurtilimab | CD40 | Cifurtilimab; SEA-CD40 | MedChemExpress, | |
| | | | #HY-P99603 | |
| ciltacabtagene | BCMA | | | Berdeja et al., 2021, |
| autoleucel | | | | Lancet 398 (10297): |
| | | | | 314-324. |
| CK6 | CD117 | | | Lebron et al., 2014, |
| | | | | Cancer Biol. Ther. |
| | | | | 15 (9): 1208-1218. |
| CL1659 | CD117 | CD117/c-kit Antibody | Novus | |
| | | (CL1657) | Biologicals, | |
| | | | #NBP2-52975 | |
| CLB-CD19 | CD19 | | | De Rie, 1989, Cell. |
| | | | | Immunol. 118: 368- |
| | | | | 381 |
| clenoliximab | CD4 | Clenoliximab | Novus | |
| | | | Biologicals, | |
| | | | #NBP2-52681H | |
| Cmab-43 | CD133 | Recombinant Anti- | Abcam, | |
| | | CD133 antibody | #AB264538 | |
| CO.44B8 | CD43 | CD43 Monoclonal | ThermoFisher, | |
| | | Antibody (CO.44B8) | #MA5-28371 | |
| cobolimab | TIM-3 | Cobolimab | MedChemExpress, | |
| | | | #HY-P99827 | |
| CPP32 4-1-18 | Sca-1 | Caspase 3 Monoclonal | ThermoFisher, | |
| | | Antibody (CPP32 4-1-18) | MA1-16843 | |
| crefmirlimab | CD8α | available from multiple sources, including: | | Farwell et al., J Nucl |
| | | Crefmirlimab | MedChemExpress, | Med 63(5): 720-726 |
| | | | #HY-P99834 | (2021); |
| | | Crefmirlimab Biosimilar - | ProteGenix, #PX- | U.S. Pat. No. 10,414,820 |
| | | Anti-CD8A mAb - | TA1828-100 | |
| | | Research Grade | | |
| CS1002 | CTLA-4 | | | Bishnoi et al., 2021, |
| | | | | Ann. Oncol. 32, |
| | | | | (Suppl. 5): S840. |
| CT041 | CLDN18.2 | | | Qi et al., 2022, Nat. |
| | | | | Med. 28, 1189- |
| | | | | 1198. |
| CT103A | BCMA | | | U.S. Pat. No. 11,026,975 |
| (CAR0085) | | | | |
| cudarolimab | OX40 | Cudarolimab | MedChemExpress, | Kuang, Z et al. |
| | | | #HY-P99836 | Cancer Immunol |
| | | | | Immunother. 2020 |
| | | | | June; 69(6): 939-950. |
| cusatuzumab | CD70 | Cusatuzumab | MedChemExpress, | Riether, C et al. Nat |
| | | | #HY-P99014 | Med. 2020 |
| | | | | September; 26(9): 1459- |
| | | | | 1467. |
| CYT-91000 | CD25 | Research Grade Anti- | AntibodySystem, | |
| | | Human CD25/IL2RA | #DHB95805 | |
| | | (CYT-91000) | | |
| dacetuzumab | CD40 | Dacetuzumab | MedChemExpress, | Hussein, M et al. |
| | | | #HY-P99015 | Haematologica. |
| | | | | 2010 |
| | | | | May; 95(5): 845-8. |
| daclizumab | CD25 | Daclizumab | MedChemExpress, | Cohan, S. L. et al. |
| | | | #HY-108738 | Biomedicines. 2019 |
| | | | | Mar. 11; 7(1):18. |
| daratumumab | CD38 | Daratumumab (anti- | SelleckChem, | |
| | | CD38) | #A2027 | |
| davoceticept | CD28 | Davoceticept | MedChemExpress, | Patnaik, A. et al. |
| | | | #HY-P99844 | JCO 40, TPS2683- |
| | | | | TPS2683(2022). |
| DF-T1 | CD43 | CD43 Antibody (DF-T1) | Biotechnology, | |
| | | | Santa Cruz | |
| | | | #sc-6256 | |
| dinutuximab | GD2 | Dinutuximab | ThermoFisher, | Mohd, A. B. et al. J |
| | | | #MA5-42000 | Res Med Sci. 2023 |
| | | | | Sep. 29; 28:71. |
| dostarlimab | PD-1 | Dostarlimab | SelleckChem, | Mirza, M.R. et al. N |
| | | | #A2835 | Engl J Med. 2023 |
| | | | | Jun. 8; 388(23): 2145- |
| | | | | 2158. |
| DS-7300A | B7H3 | | | Yamato et al., 2022, |
| | | | | Mol. Cancer Ther. |
| | | | | 21(4): 635-646. |
| EasySep ™ | | EasySep ™ Human T | Stemcell | |
| | | Cell Isolation Kit | Technologies, | |
| | | | #100-0695 | |
| efungumab | Stro-4 | Efungumab | MedChemExpress, | Karwa, R. et al. Ann |
| | | | #HY-P9962 | Pharmacother. 2009 |
| | | | | November; 43(11): 1818-23. |
| ektomab | GD2 | | | Ruf, P. et al. J |
| | | | | Transl Med. 2012 |
| | | | | Nov. 7; 10: 219. |
| ELISA MAX ™ | TNF-α | ELISA MAX ™ Deluxe | BioLegend, | |
| Deluxe Set | | Set Human TNF-α kit | #430204 | |
| Human TNF-α kit | | | | |
| elranatamab | CD3 | Research Grade | AntibodySystem, | Lesokhin, A. M. et |
| | | Elranatamab | #DHF92406 | al. Nat Med. 2023 |
| | | | | September; 29(9): 2259- |
| | | | | 2267. |
| EMB-02 | LAG-3 | | | Day et al., 2023, |
| | PD-1 | | | Ann. Oncol. 34 |
| | | | | (Suppl. 2): S625. |
| EMB-06 | BCMA | | | US 2023/0002489 |
| EMB-09 | OX40 | | | Li et al., 2023, Antib. |
| | PD-L1 | | | Ther. 6 (Suppl 1): |
| | | | | tbad014.008. |
| EMD 273063 | GD2 | Anti-GD2 (EMD | SelleckChem, | |
| | | 273063) | #A2916 | |
| encelimab | LAG-3 | Encelimab | MedChemExpress, | Perez-Santos, M. et |
| | | | #HY-P99922 | al. Expert Opin Drug |
| | | | | Discov. 2022 |
| | | | | December; 17(12): 1341- |
| | | | | 1355. |
| enoblituzumab | B7H3 | Anti-Human CD276 | Creative Biolabs, | |
| | | Recombinant Antibody | #TAB-424CQ | |
| EOS-448 | TIGIT | | | Cuende et al., 2022, |
| | | | | Cancer Res. 82 |
| | | | | (12_Supplement): |
| | | | | LB189. |
| EP322Y | CD45 | Anti-CD45 antibody | Abcam, | |
| | | [EP322Y] | #ab40763 | |
| EPR8913(2) | SUSD2 | Anti-SUSD2 antibody | Abcam, | |
| | | [EPR8913(2)] | #ab182147 | |
| EPR20545 | CD68 | Anti-CD68 antibody | Abcam, | |
| | | [EPR20545] | #ab213363 | |
| EPR26538-16 | CD8ß | Recombinant Anti-CD8 | Abcam, | |
| | | beta antibody | #ab300067 | |
| | | [EPR26538-16] | | |
| EPR26948-19 | FCRL5 | Anti-FCRL5 antibody | Abcam, | |
| | | [EPR26948-19] - BSA | #ab307569 | |
| | | and Azide free | | |
| | | (Detector) | | |
| EPR26948-67 | FCRL5 | Anti-FCRL5 antibody | Abcam, | |
| | | [EPR26948-67] - BSA | #ab307568 | |
| | | and Azide free | | |
| | | (Capture) | | |
| EPR27365-87 | | Anti-FCRL5 antibody | Abcam, | |
| | | [EPR27365-87] | #ab307730 | |
| epratuzumab | CD22 | Anti-Human CD22 | Creative Biolabs, | |
| | | Recombinant Antibody | #TAB-176 | |
| ET200-31 | FCRL5 | | | U.S. Pat. No. 10,913,796 |
| ET200-39 | | | | |
| ET200-69 | | | | |
| ET200-104 | | | | |
| ET200-105 | | | | |
| ET200-109 | | | | |
| ET200-117 | | | | |
| etigilimab | TIGIT | | | Mettu et al., 2022, |
| | | | | Clin. Cancer. Res. |
| | | | | 28(5): 882-892. |
| ezabenlimab | PD-1 | Ezabenlimab | MedChemExpress, | Zettl, M. et al. |
| | | | #HY-P99610 | Oncoimmunology. |
| | | | | 2022 Jun. |
| | | | | 16; 11(1): 2080328. |
| F10-89-4 | CD45 | Anti-CD45 antibody | Abcam, | |
| | | [F10-89-4] - | #ab30470 | |
| | | Hematopoietic Stem | | |
| | | Cell Marker | | |
| F119 | FCRL5 | | | Ise et al. (2005) |
| | | | | Clin. Cancer Res. |
| | | | | 11(1): 87-96. |
| F25 | FCRL5 | Anti-FcRH5 Antibody, | Millipore Sigma, | |
| | | clone F25 | #MABF2103 | |
| F26 | FCRL5 | | | Franco et al. (2013) |
| | | | | J. Immunol. 190(11): |
| | | | | 5739-5746. |
| favezelimab | LAG-3 | Favezelimab | MedChemExpress, | Gareth, P. et al. |
| | | | #HY-P99613 | Cancer Res1 July |
| | | | | 2019; 79 |
| | | | | (13_Supplement): |
| | | | | CT106. |
| felzartamab | CD38 | Felzartamab | MedChemExpress, | |
| | | | #HY-P99616 | |
| FHVH33 | BCMA | | | WO 2019/006072 |
| fianlimab | LAG-3 | Fianlimab | MedChemExpress, | Omid, H. et al. JCO |
| | | | #HY-P99617 | 39, 9515-9515 |
| | | | | (2021). |
| FMC63 | CD19 | | | Nicholson et al., |
| | | | | 1997, Mol. Immun. |
| | | | | 34(16-17): 1157- |
| | | | | 1165; WO |
| | | | | 2018/213337, WO |
| | | | | 2015/187528 |
| forimtamig | GPRC5D | | | Harrison et al. |
| | | | | (2023). Clin. |
| | | | | Lymphoma |
| | | | | Myeloma Leuk. 23 |
| | | | | (Suppl. 2): S3-4. |
| FR4D11 | CD10 | Anti-Human CD10 | Stemcell | |
| | | Antibody, Clone | Technologies, | |
| | | FR4D11 | #60149 | |
| FR104CD | CD28 | Recombinant Anti- | Antibodies.com, | |
| | | CD28 Antibody | #A323936 | |
| | | [FR104] | | |
| FS118 | LAG-3 | | | Kraman et al., 2020, |
| | | | | Clin. Cancer Res. |
| | | | | 26 (13): 3333-3344. |
| FS120 | OX40 | | | Gaspar et al., 2020, |
| | CD137 | | | Cancer Immunol. |
| | | | | Res. 8 (6): 781-793. |
| G.813.2 | CD117 | Phospho-c-Kit (Tyr703) | ThermoFisher, | |
| | | Monoclonal Antibody | #MA5-14830 | |
| | | (G.813.2) | | |
| ganglidiximab | GD2 | | | Eger et al., 2016, |
| | | | | PLoS One 11 (3): |
| | | | | e0150479. |
| GEN-3014 | CD38 | | | Hiemstra et al., |
| | | | | 2023, EBioMedicine |
| | | | | 93: 104663. |
| geptanolimab | PD-1 | Geptanolimab | AntibodySystem, | Shi, Y. et al. Clin |
| | | | #DHH02224 | Cancer Res. 2020 |
| | | | | Dec. 15; 26(24): 6445- |
| | | | | 6452. doi: |
| | | | | 10.1158/1078- |
| | | | | 0432.CCR-20-2819. |
| | | | | Epub 2020 Oct. 12. |
| | | | | PMID: 33046518. |
| GIGA-564 | CTLA-4 | | | Stone, E. et al. |
| | | | | Journal for |
| | | | | ImmunoTherapy of |
| | | | | Cancer, 2022; 10. |
| givastomig | CLDN18.2 | | | Gao et al., 2023, J. |
| | | | | Immunother. Cancer |
| | | | | 11(6): e006704. |
| GN1412 | CD28 | Theralizumab | MedChemExpress, | Hussain, K. et al. |
| | | | #HY-P9975 | Blood. 2015 Jan. |
| | | | | 1; 125(1): 102-10. |
| gresonitamab | CLDN18.2 | Gresonitamab | MedChemExpress, | |
| | | | #HY-P99350 | |
| grisnilimab | CD7 | Grisnilimab | MedChemExpress, | Scharnhorst, V, et |
| | | | #HY-P99650 | al. Gene. 2001 Aug. |
| | | | | 8; 273(2): 141-61. |
| | | | | doi: 10.1016/s0378- |
| | | | | 1119(01)00593-5. |
| | | | | PMID: 11595161. |
| GSK2831781 | LAG-3 | | | Clinical Trial ID: |
| | | | | NCT03893565 |
| GSK3174998 | OX40 | | | Infante, J. R. et al. |
| | | | | JCO 34, TPS3107- |
| | | | | TPS3107 (2016). |
| GWN323 | GITR | | | Piha-Paul, S. A. et |
| | | | | al. Journal for |
| | | | | Immuno Therapy of |
| | | | | Cancer |
| | | | | 2021; 9: e002863. |
| h4C8 | CD34 | Anti-Human CD34 | Creative Biolabs, | |
| | | Recombinant Antibody | #TAB-149LC- | |
| | | Fab Fragment (h4C8) | F(E) | |
| H22 | CD64 | | | Hristodorov, D. et al. |
| | | | | MAbs. |
| | | | | 2014; 6(5): 1283-9. |
| | | | | doi: |
| | | | | 10.4161/mabs.32182. |
| | | | | PMID: 25517313; |
| | | | | PMCID: PMC4622438. |
| H44 | IL-18Rα | APC anti-human | Biolegend, | |
| | | CD218a (IL-18Rα) | #313814 | |
| | | Antibody | | |
| H65 | CD5 | CD5 Antibody | Novus | |
| | | | Biologicals, | |
| | | | #NB100-2667 | |
| hB-F5 | CD4 | NeuroMab ™ Anti-CD4 | Creative Biolabs, | |
| | | BBB Shuttle Antibody, | #NRZP-1022- | |
| | | Clone hB-F5 | ZP3717 | |
| HBM1007 | CD73 | | | Gan et al., 2020, |
| | | | | 2020 AACR Poster |
| | | | | #6056. |
| HBM4003 | CTLA-4 | | | Shanzhi, G. et al. |
| | | | | JCO 40, 2641-2641 |
| | | | | (2022). |
| HC34LC14 | CD117 | c-Kit Recombinant | ThermoFisher, | |
| | | Rabbit Monoclonal | #701494 | |
| | | Antibody (HC34LC14) | | |
| HCD27.15 | CD27 | | | U.S. Pat. No. 9,527,916 |
| HD37 | CD19 | | | Pezutto et al., 1987, |
| | | | | J. Immunol. 138(9): |
| | | | | 2793-2799 |
| HE3 | CD5 | | | U.S. Pat. No. 5,770,196 |
| HFB301001 | OX40 | | | Fulton, R. et al. |
| | | | | Cancer Res 1 July |
| | | | | 2021; 81 |
| | | | | (13_Supplement): |
| | | | | 1882. |
| HI30 | CD45 | Purified anti-human | BioLegend, | |
| | | CD45 Antibody | #304001 | |
| HIT8a | CD8α | Purified anti-human | BioLegend, | |
| | | CD8a Antibody | #300901 | |
| HLX26 | LAG-3 | | | Rujiao, L. et al. JCO |
| | | | | 41, e14671-e14671 |
| | | | | (2023). |
| HLX51 | OX40 | | | Clinical Trial ID: |
| | | | | NCT05788107 |
| HM48-1 | CD48 | CD48 Monoclonal | ThermoFisher, | |
| | | Antibody (HM48-1), | #17-0481-82 | |
| | | APC, eBioscience ™ | | |
| HPAB-0260-YJ | CD200 | Human Anti-CD200 | Creative Biolabs, | |
| | | Recombinant Antibody | #HPAB-0260-YJ- | |
| | | | F(E) | |
| HPAB-3334LY | PODXL | Human Anti-PODXL | Creative Biolabs, | |
| | | Recombinant Antibody | #HPAB-3334LY | |
| HPAB-MO612-YC | PODXL | Human Anti-LAM | Creative Biolabs, | |
| | | Recombinant Antibody | #HPAB-M0561- | |
| | | | YC | |
| HPN217 | BCMA | | | U.S. Pat. No. 11,136,403 |
| Hu001-MMAE | CD73 | | | Jin et al., 2020, Mol. |
| | | | | Cancer Ther. 19 |
| | | | | (11): 2340-2352. |
| hu3G8-5.1-N297Q | CD16a | | | U.S. Pat No. 7,351,803 |
| hu14.18k322A | GD2 | Anti-Human GD2 | Creative Biolabs, | |
| | | Recombinant Antibody | #TAB-304CL | |
| | | (hu14.18K322A) | | |
| Hu222 | OX40 | | | U.S. Pat No. 9,006,399 |
| huAA98 | CD146 | Anti-Human MCAM | Creative Biolabs, | |
| | | Recombinant Antibody | #TAB-444MZ | |
| | | (huAA98) | | |
| Huly-m2 | CD7 | CD7 (T-Cell Leukemia | ThermoFisher, | |
| | | Marker) Monoclonal | #924-MSM2- | |
| | | Antibody (HuLy-m2) | P1ABX | |
| HuMab 611 | CD64 | | | WO 2006/002438 |
| Human IgG1 poly- | IgG1 | Human IgG1 Poly- | Medna Scientific, | |
| specificity control | | Specificity Control | #H1308 | |
| antibody | | Antibody | | |
| Human IgG4 | IgG4 | Human IgG4 Isotype | Medna Scientific, | |
| isotype control | | Control Antibody | #H1314 | |
| antibody | | | | |
| humanized AT-10 | CD32A | CD32 Antibody | Biorad, | |
| | | | #MCA1075 | |
| humanized IV.3 | CD32A | Anti-Human CD32 | StemCell | |
| | | Antibody, Clone IV.3 | Technologies, | |
| | | | #60012 | |
| HuMax-TAC | CD25 | Camidanlumab | MedChemExpress, | |
| | | | #HY-P99233 | |
| huRH105 | CD105 | Anti-CD105 [huRH105- | Absolute | |
| | | 1] | Antibody, | |
| | | | #Ab04017 | |
| IAB22M | CD8 | | | U.S. Pat No. 11,254,744 |
| IAb_CysDb3b_CD8 | CD8 | | | U.S. Pat No. 10,414,820 |
| IAb_M1b_CD8 | CD8 | | | |
| IAb_M2b_CD8 | CD8 | | | |
| ibalizumab | CD4 | Ibalizumab | MedChemExpress, | |
| | | | #HY-P99028 | |
| IBI110 | LAG-3 | | | Xu et al., 2022, J. |
| | | | | Clin. Oncol. 40 |
| | | | | (16_suppl): 2650. |
| IBI310 | CTLA-4 | | | Zhou et al., 2022, J. |
| | | | | Clin. Oncol. 40 |
| | | | | (4_suppl): 421. |
| IBI318 | PD-1 | Reozalimab | MedChemExpress, | |
| | | (LY3434172) | #HY-P9996 | |
| IBI323 | LAG-3 | | | Ni et al., 2020, |
| | | | | Cancer Res. 80 |
| | | | | (16_Supplement): |
| | | | | 3270. |
| IBI325 | CD73 | | | Zhou et al., 2023, |
| | | | | Int. J. Biol. |
| | | | | Macromol. 229: 158- |
| | | | | 167. |
| IBI343 | CLDN18.2 | | | Yu et al., 2024, J. |
| | | | | Clin. Oncol. 42 (16 |
| | | | | Suppl.), 3037. |
| ibritumomab | CD20 | Ibritumomab Biosimilar - | ProteoGenix, | |
| | | Anti- | #PX-TA1620 | |
| | | MS4A1(CD20, MS4A-1) | | |
| | | mAb - Research Grade | | |
| idecabtagene | BCMA | | | Munshi et al., 2021, |
| vicleucel | | | | N. Engl. J. Med. |
| | | | | 384: 705-716. |
| ieramilimab | LAG-3 | Ieramilimab | MedChemExpress, | Schöffski, P et al. J |
| | | | #HY-P99027 | Immunother Cancer. |
| | | | | 2022 |
| | | | | February; 10(2): e003776. |
| IL-A116 | CD45RO | CD45RO Monoclonal | ThermoFisher, | |
| | | Antibody (IL-A116) | #MA5-28403 | |
| imaprelimab | CD146 | Imaprelimab | MedChemExpress, | Pelletier, J. P. R. Et |
| | | | #HY-P99658 | al. Immunologic |
| | | | | Concepts in |
| | | | | Transfusion |
| | | | | Medicine, Elsevier, |
| | | | | 2020, 251-348. |
| INBRX-106 | OX40 | | | Clinical Trial ID: |
| | | | | NCT04198766 |
| INCA00186 | CD73 | | | Stewart, S. et al. |
| | | | | Cancer Res 1 July |
| | | | | 2021; 81 |
| | | | | (13_Supplement): |
| | | | | LB174. |
| INCA32459 | LAG-3 | | | Clinical Trial ID: |
| | | | | NCT05577182 |
| INCAGN1949 | OX40 | | | Davis, E. J. et al. J |
| | | | | Immunother Cancer. |
| | | | | 2022 |
| | | | | October; 10(10): e004235. |
| | | | | doi: 10.1136/jitc- |
| | | | | 2021-004235. |
| | | | | PMID: 36316061; |
| | | | | PMCID: PMC9628691. |
| INCAGN2385 | LAG-3 | | | Clinical Trial ID: |
| | | | | NCT03538028 |
| INCAGN02390 | TIM-3 | | | Clinical Trial ID: |
| | | | | NCT03652077 |
| inezetamab | CD4 | Inezetamab | MedChemExpress, | |
| | | | #HY-P99663 | |
| inolimomab | CD25 | Research Grade | Antibody System, | |
| | | Inolimomab | #DHB95804 | |
| inotuzumab | CD22 | Humanized Anti-CD22 | Creative Biolabs, | |
| | | Recombinant Antibody | #TAB-198 | |
| | | (clone Inotuzumab) | | |
| IPH5301 | CD73 | | | Gonçalves et al., |
| | | | | 2022, Immuno- |
| | | | | Oncology and |
| | | | | Technology 16 (S1): |
| | | | | 199TiP. |
| ipilimumab | CTLA-4 | Ipilimumab | MedChemExpress, | |
| | | | #HY-P9901 | |
| IPO-3 | CD150 | Anti-SLAM/CD150 | Abcam, #ab2604 | |
| | | antibody [IPO-3] | | |
| isatuximab | CD38 | Isatuximab (anti-CD38) | SelleckChem, | |
| | | | #A2039 | |
| iscalimab | CD40 | Iscalimab | MedChemExpress, | |
| | | | #HY-P99670 | |
| ispectamab | BCMA | | | US 2021/0130483 |
| IT1208 | CD4 | | | Shitara, K et al. J |
| | | | | Immunother Cancer. |
| | | | | 2019 Jul. |
| | | | | 24; 7(1): 195. |
| ivuxolimab | OX40 | Ivuxolimab | MedChemExpress | Diab, A et al. Clin |
| | | | (MCE), #HY- | Cancer Res. 2022 |
| | | | P99159 | Jan. 1; 28(1): 71-83. |
| JAB-BX102 | CD73 | | | Clinical Trial ID: |
| | | | | NCT05174585 |
| JK08 | CTLA-4 | | | Kotecki, et al., 2023, |
| | | | | Ann. Oncol. 34(S2), |
| | | | | pg. S635. |
| JM1-24-3 | CD146 | | | Feng et al., 2020, J. |
| | | | | Exp. Clin. Cancer |
| | | | | Res. 39: 273. |
| JM7A4 | IL-15Rα | available from multiple sources, including: | | |
| | | Anti-IL-15RA antibody | Abcam, | |
| | | [JM7A4] | #ab91270 | |
| | | IL-15R alpha Antibody | Novus | |
| | | (JM7A4) - BSA Free | Biologicals, | |
| | | | #NBP1-43238 | |
| JMW-3B3 | CTLA-4 | Afuco ™ Anti-Human | Creative Biolabs, | |
| | | CTLA4 ADCC | #AFC-185CL | |
| | | Recombinant Antibody | | |
| | | (JMW-3B3), ADCC | | |
| | | Enhanced | | |
| JNJ-64164711 | GITR | Research Grade Anti- | \|#DHJ89808 | Holland et al., 2018, |
| | | Human CD357/TNFRSF18/GITR | AntibodySystem, | Cancer Res. 78 |
| | | | | (13_Supplement): |
| | | | | 3813. |
| JS007 | CTLA-4 | | | Guan et al., 2023, |
| | | | | MAbs 15(1): |
| | | | | 2153409. |
| JTX-2011 | ICOS | Vopratelimab | MedChemExpress, | |
| | | | #HY-P99382 | |
| K45 | CD117 | c-Kit Monoclonal | ThermoFisher, | |
| | | Antibody (K45) | #MA5-12944 | |
| K117 | CD90 | Anti-CD90 Antibody, | RayBiotech, | |
| | | Purified | #136-00048 | |
| KD6001 | CTLA-4 | | | Chen et al., 2024, |
| | | | | Cancer Res. 84 |
| | | | | (6_Suppl.): 1350. |
| keliximab | CD4 | available from multiple sources, including: | | |
| | | Keliximab | MedChemExpress, | |
| | | | #HY-P99680 | |
| | | Keliximab (CD4) - | ProSci, #10-178 | |
| | | Research Grade | | |
| | | Biosimilar | | |
| Ki-M7 | CD68 | available from multiple sources, including: | | |
| | | CD68 antibody \| Ki-M7 | Bio-Rad, | |
| | | | #MCA2375F | |
| | | CD68 Monoclonal | ThermoFisher, | |
| | | Antibody (Ki-M7), FITC | #MA1-82715 | |
| kit2c75 | CD117 | CD117 (c-Kit) | ThermoFisher, | |
| | | Monoclonal Antibody | #MA5-28805 | |
| | | (kit2c75), PE | | |
| KM666 | GD2 | | | Nakamura et al., |
| | | | | 2001, Cancer |
| | | | | Immunol. |
| | | | | Immunother. 50, |
| | | | | 275-284. |
| KN044 | CTLA-4 | | | Clinical Trial ID: |
| | | | | NCT04126590 |
| L17F12 | CD5 | available from multiple sources, including: | | |
| | | Purified anti-human | BioLegend, | |
| | | CD5 Antibody | #364001 | |
| | | CD5 Antibody | Santa Cruz | |
| | | (L17F12) | Biotechnology, | |
| | | | #sc-18898 | |
| LAMP4-824 | CD68 | available from multiple sources, including: | | |
| | | CD68 Monoclonal | Biotium, #0824 | |
| | | Mouse Antibody | | |
| | | (LAMP4/824) | | |
| | | CD68 Monoclonal | ThermoFisher, | |
| | | Antibody (LAMP4, 824) | #968-MSM4-P1 | |
| LB1410 | TIM-3 | | | Liu, 2023, J. Clin. |
| | | | | Oncol. 41 |
| | | | | (16_suppl): |
| | | | | TPS2663. |
| LCAR-B38M | BCMA | | | Zhao et al., 2018, J. |
| | | | | Hematol. Oncol. |
| | | | | 11(1): 141; |
| | | | | WO 2018/028647 |
| LCAR-C18S | CLDN18.2 | | | Zhen et al., 2023, J. |
| (LB1908) | | | | Clin. Oncol. 41 (4 |
| | | | | suppl.), TPS480. |
| Leu16 | CD20 | | | Wu et al., 2001, |
| | | | | Protein Engineering. |
| | | | | 14(12): 1025-1033 |
| linvoseltamab | BCMA | | | U.S. Pat No. 11,919,965 |
| lisocabtagene | CD19 | | | Abramson et al., |
| maraleucel | | | | 2020, Lancet 396: |
| | | | | 839-852. |
| LM-302 | CLDN18.2 | | | Huang et al., 2022, |
| | | | | Eur. J. Cancer 174 |
| | | | | (Suppl. 1): S41-S42. |
| LOP628 | CD117 | Anti-cKit-maitansine | Creative Biolabs, | |
| | | ADC (LOP628) | #ADC-L039 | |
| lorigerlimab | PD-1 | | | Luke et al., J. Clin. |
| | | | | Oncol. 41 (6_suppl): |
| | | | | 155. |
| lorvotuzumab | CD56 | available from multiple sources, including: | | |
| | | Lorvotuzumab | MedChemExpress, | |
| | | | #HY-P99372 | |
| | | Lorvotuzumab | ThermoFisher, | |
| | | Recombinant Human | #MA5-41708 | |
| | | Monoclonal Antibody | | |
| LT-2 | CD2 | CD2 Monoclonal | ThermoFisher, | |
| | | Antibody (LT2) | #MA1-10131 | |
| LT-8 | CD8 | available from multiple sources, including: | | |
| | | Mouse monoclonal | Antibodies.com, | |
| | | [LT8] antibody to CD8 | #A242891 | |
| | | (PE) | | |
| | | Monoclonal Anti-CD8 | Millipore Sigma, | |
| | | antibody | #SAB4701064 | |
| lucatumumab | CD40 | available from multiple sources, including: | | |
| | | Lucatumumab | MedChemExpress, | |
| | | | #HY-P99167 | |
| | | Anti-CD40 | Creative Biolabs, | |
| | | Recombinant Antibody | #TAB-759 | |
| | | (Lucatumumab) | | |
| lulizumab | CD28 | available from multiple sources, including: | | |
| | | Lulizumab | ThermoFisher, | |
| | | Recombinant Human | #MA5-42032 | |
| | | Monoclonal Antibody | | |
| | | Humanized Anti-CD28 | Creative Biolabs, | |
| | | Recombinant Antibody | #TAB-H46 | |
| | | (clone Lulizumab) | | |
| LVGN6051 | CD137 | | | Qi et al., 2019, Nat. |
| | | | | Commun. 10(1): |
| | | | | 2141. |
| LY75_A1 | CD205 | LY75/DEC-205 Protein | Antibodies | |
| | | (AA 28-1667) (His tag) | Online, | |
| | | | #ABIN7275222 | |
| LY3321367 | TIM-3 | | | Harding et al., 2021, |
| | | | | Clin. Cancer Res. |
| | | | | 27(8): 2168-2178. |
| LY3415244 | TIM-3 | | | Hellmann et al., |
| | | | | 2021, Clin. Cancer |
| | | | | Res. 27(10): 2773- |
| | | | | 2781. |
| M108 | CLDN18.2 | | | Jin et al., 2024, J. |
| (FG-M108) | | | | Clin. Oncol. 42 (16 |
| | | | | suppl.), 4142. |
| M971 | CD22 | Anti-Siglec-2/CD22 | SelleckChem, | |
| | | (NCI m971) | #A3057 | |
| mAb19 | CD73 | | | Wurm et al., 2021, |
| | | | | Mol. Cancer Ther. |
| | | | | 20(11): 2250-2261 |
| MAB107 | CD11b | available from multiple sources, including: | | |
| | | Anti-CD11b/CD18 | Millipore Sigma, | |
| | | Antibody, clone | #MABF2085 | |
| | | mAb107 | | |
| | | Anti-human IgE mAb | Mabtech, #3810- | |
| | | (107), unconjugated | 3 | |
| MAB1472-100 | IL-15Rα | available from multiple sources, including: | | |
| | | Human IL-15R alpha | R&D Systems, | |
| | | Antibody | #MAB1472 | |
| | | IL-15R alpha Antibody | Novus | |
| | | (1020456) | Biologicals, | |
| | | [Unconjugated] | #MAB1472 | |
| MAB5511 | IL-15Rα | Mouse IL-15R alpha | R&D Systems, | |
| | | Antibody | #MAB5511 | |
| MAI1738 | PODXL | Research Grade Anti- | AntibodySystem, | |
| | | Human PODXL | #DHA 16801 | |
| | | (MAI1738) | | |
| MAT304 | CD5 | Research Grade Anti- | AntibodySystem, | |
| | | Human CD5 (MAT 304) | #DHC18804 | |
| MAX.16H5 | CD4 | | | Guse et al., 1994, J. |
| | | | | Chromatogr. A 661: |
| | | | | 13-23. |
| MB-106 | CD20 | Fred Hutchinson Cancer Research Center | | Shadman et al., |
| | | | | 2019, Blood |
| | | | | 134(Suppl.1): 3235 |
| MC631 | SSEA-3 | available from multiple sources, including: | | |
| | | Anti-SSEA3 antibody | Abcam, | |
| | | [MC631] | #ab16286 | |
| | | Anti-Mouse SSEA-3 | StemCell | |
| | | Antibody, Clone MC- | Technologies, | |
| | | 631 | #60061.1 | |
| MC-813-70 | SSEA-4 | available from multiple sources, including: | | |
| | | SSEA4 Monoclonal | ThermoFisher, | |
| | | Antibody (MC-813-70) | #MA1-021 | |
| | | Anti-Human SSEA-4 | StemCell | |
| | | Antibody, Clone MC- | Technologies, | |
| | | 813-70 | #60062 | |
| MCARH109 | GPRC5D | | | Mailankody et al., N |
| | | | | Engl J Med. |
| | | | | 387(13): 1196-1206 |
| | | | | (2022) |
| MDE-8 | CD32A | Anti-CD32 [MDE-8] | Absolute | |
| | | | Antibodies, | |
| | | | #Ab02072 | |
| MDX-1203 | CD70 | | | Owonikoko et al., |
| | | | | 2014, J. Clin. Oncol. |
| | | | | 32: 5s, (suppl; abstr |
| | | | | 2558). |
| MDX-1411 | CD70 | Anti-CD70 monoclonal | Creative Biolabs, | |
| | | antibody (MDX-1411) | #Gly-008CL | |
| MEDI0680 | PD-1 | | | Naing et al., 2019, J. |
| | | | | Immunother. Cancer |
| | | | | 7: 225. |
| MEDI1873 | GITR | | | Tigue et al., 2017, |
| | | | | Oncoimmunology |
| | | | | 6(3): e1280645. |
| MEDI2228 | BCMA | | | U.S. Pat No. 10,988,546 |
| MEDI5752 | PD-1 | | | Dovedi et al., 2021, |
| | | | | Cancer Discov. |
| | | | | 11(5): 1100-1117. |
| MEDI6383 | OX40 | | | Hammond et al., |
| | | | | 2015, J. Clin. Oncol. |
| | | | | 33 (15_suppl): 3056. |
| MEDI6469 | OX40 | | | Leidner et al., 2015, |
| | | | | J. Clin. Oncol. 33 |
| | | | | (15_suppl): TPS6083. |
| MEM-28 | CD45 | Anti-CD45 antibody | Abcam, #ab8216 | |
| | | [MEM-28] | | |
| MEM-31 | CD8 | available from multiple sources, including: | | |
| | | CD8 Monoclonal | ThermoFisher, | |
| | | Antibody (MEM-31) | #MA1-19082 | |
| | | Monoclonal Anti-CD8 | SigmaAldrich, | |
| | | antibody produced in | #SAB4700084 | |
| | | mouse | | |
| MEM-55 | CD45RB | Anti-CD45RB antibody | Abcam, #ab8218 | |
| | | [MEM-55] | | |
| MEM-59 | CD43 | CD43 Monoclonal | ThermoFisher, | |
| | | Antibody (MEM-59) | #MA1-19009 | |
| MEM-65 | CD2 | available from multiple sources, including: | | |
| | | CD2 Monoclonal | ThermoFisher, | |
| | | Antibody (MEM-65) | #MA1-19070 | |
| | | Anti-CD2 Antibody | Antibodies.com, | |
| | | [MEM-65] | #A86387 | |
| MEM-87 | CD8 | available from multiple sources, including: | | |
| | | Anti-CD8 Antibody | Antibodies.com, | |
| | | (MEM-87) | #A85681 | |
| | | Monoclonal Anti-CD8 | SigmaAldrich, | |
| | | antibody produced in | #SAB4700089 | |
| | | mouse | | |
| MEM-102 | CD48 | CD48 Monoclonal | ThermoFisher, | |
| | | Antibody (MEM-102) | #MA1-19119 | |
| mezagitamab | CD38 | Mezagitamab | MedChemExpress, | |
| | | | #HY-P99730 | |
| MGC018 | B7H3 | | | Scribner et al., |
| | | | | 2020, Mol. Cancer |
| | | | | Ther. 19(11): 2235- |
| | | | | 2244. |
| MGTA-117 | CD117 | | | Westervelt et al., |
| | | | | 2022, Blood 140 |
| | | | | (Supplement 1): |
| | | | | 2117-2119. |
| MiK-Beta-1 | CD122 | CD122 Monoclonal | ThermoFisher, | |
| | | Antibody (MIK-Beta 1) | #MA1-35896 | |
| miptenalimab | LAG-3 | Miptenalimab | MedChemExpress, | |
| | | | #HY-P99736 | |
| mitazalimab | CD40 | | | Calvo et al., 2019, J. |
| | | | | Clin. Oncol. 37 |
| | | | | (15_suppl): 2527. |
| MK-1248 | GITR | | | Geva et al., 2020, |
| | | | | Cancer 126(22): |
| | | | | 4926-4935. |
| MK-4166 | GITR | | | Sukumar et al., |
| | | | | 2017, Cancer Res. |
| | | | | 77 (16): 4378-4388. |
| MORAb-028 | GD2 | MORAb-028 | ThermoFisher, | |
| | | Recombinant Human | #MA5-41810 | |
| | | Monoclonal Antibody | | |
| moxetumomab | CD22 | Anti-CD22 | Creative Biolabs, | |
| | | (Moxetumomab | #ADC-W-800 | |
| | | pasudotox)-SMCC- | | |
| | | DM1 ADC | | |
| mShad150 | CD150 | CD150 Monoclonal | ThermoFisher, | |
| | | Antibody (mShad150), | #12-1502-82 | |
| | | PE, eBioscience ™ | | |
| M-T413 | CD4 | | | Rieber et al., 1992, |
| | | | | PNAS 89(22): |
| | | | | 10792-6. |
| MT95-4 | CD13 | | | WO 2021/072312 |
| MRTX1011A | CD4 | | | Scheerens et al., |
| | | | | 2011, Arthritis Res. |
| | | | | Ther. 13 (5): R177. |
| MT807-R1 | CD8α | Anti-CD8 alpha | NIH Nonhuman | Nakamura-Hoshi et |
| | | (MT807R1) | Primate Reagent | al., 2024, Mol. Ther. |
| | | | Resources, | 32 (7): 1-12. |
| | | | #AB_2716320 | |
| mupadolimab | CD73 | Mupadolimab | MedChemExpress, | |
| | | | #HY-P99181 | |
| My10 | CD34 | Anti-CD34 antibody | Abcam, | |
| | | [My10] | #ab245689 | |
| naxitamab | GD2 | Naxitamab | MedChemExpress, | |
| | | | #HY-P99206 | |
| Nbl57 | CD13 | | | WO 2021/072312 |
| ND-742 | SSEA-3 | Anti-SSEA-3 | Creative | |
| | | monoclonal antibody | Diagnostics, | |
| | | | #DMAB12907 | |
| nivatrotamab | GD2 | Nivatrotamab | MedChemExpress, | |
| | | | #HY-P99757 | |
| nivolumab | PD-1 | available from multiple sources, including: | | |
| | | Nivolumab (anti-PD-1) | SelleckChem, | |
| | | | #A2002 | |
| | | Nivolumab | Evidentic, | |
| | | | #8006657 | |
| NN2101 | CD117 | | | Kim et al., 2020, Int. |
| | | | | J. Biol. Macromol. |
| | | | | 159: 66-78. |
| nofazinlimab | PD-1 | Nofazinlimab | MedChemExpress, | |
| | | | #HY-P99758 | |
| nurulimab | CTLA-4 | available from multiple sources, including: | | |
| | | Nurulimab | MedChemExpress, | |
| | | | #HY-P99760 | |
| | | Nurulimab (Anti-CTLA- | SelleckChem, | |
| | | 4/CD152 | #A2859 | |
| NZV930 | CD73 | | | Fu et al., 2022, |
| | | | | Cancer Res. |
| | | | | 82(12_Suppl), |
| | | | | CT503. |
| obinutuzumab | CD20 | available from multiple sources, including: | | |
| | | Obinutuzumab | MedChemExpress, | |
| | | | #HY-P9910 | |
| | | Obinutuzumab (anti- | SelleckChem, | |
| | | CD20) | #A2023 | |
| | | Obinutuzumab | ThermoFisher, | |
| | | Monoclonal Antibody | #A01946-40 | |
| | | (16B7) | | |
| ocrelizumab | CD20 | available from multiple sources, including: | | |
| | | Ocrelizumab | MedChemExpress, | |
| | | | #HY-P9960 | |
| | | Ocrelizumab (Anti- | SelleckChem, | |
| | | CD20) | #A2561 | |
| | | Ocrelizumab | ThermoFisher, | |
| | | Recombinant Human | #MA5-41829 | |
| | | Monoclonal Antibody | | |
| odronextamab | CD20 | available from multiple sources, including: | | |
| | CD3 | Odronextamab | MedChemExpress, | |
| | | | #HY-P99038 | |
| | | Odronextamab | ThermoFisher, | |
| | | Recombinant Human | #MA5-42283 | |
| | | Monoclonal Antibody | | |
| ofatumumab | CD20 | available from multiple sources, including: | | |
| | | Ofatumumab | MedChemExpress, | |
| | | | #HY-P9961 | |
| | | Ofatumumab Biosimilar - | ProteoGenix, | |
| | | Anti-MS4A1, CD20 | #PX-TA1146 | |
| | | mAb - Research | | |
| | | Grade | | |
| | | Ofatumumab | ThermoFisher, | |
| | | Recombinant Human | #MA5-41813 | |
| | | Monoclonal Antibody | | |
| OKT1 | CD5 | CD5 Unconjugated | Biohippo, | |
| | | OKT1 IgG1k (RUO) | #BHA19900487 | |
| OKT3 | CD3 | CD3 Monoclonal | ThermoFisher, | |
| (muromonab- | | Antibody (OKT3), | #14-0037-82 | |
| CD3) | | eBioscience ™ | | |
| OKT4 | CD4 | Brilliant Violet 650 ™ | BioLegend, | |
| | | anti-human CD4 | #317436 | |
| | | Antibody | | |
| OKT8 | CD8α | CD8α Monoclonal | ThermoFisher, | |
| | | Antibody (OKT8) | #14-0086-80 | |
| OKT11 | CD2 | available from multiple sources, including: | | U.S. Pat No. 4,364,937 |
| | | OKT11 | ATCC, #CRL- | |
| | | | 8027 | |
| | | | MilliporeSigma, | |
| | | | #86050802 | |
| oleclumab | CD73 | Oleclumab | MedChemExpress, | |
| | | | #HY-P99039 | |
| ONC-392 | CTLA-4 | | | Rolfo et al., 2020, J. |
| | | | | Clin. Oncol. 38 |
| | | | | (15_suppl): |
| | | | | TPS3159. |
| OriCAR-017 | GPRC5D | | | Rodriguez-Otero et |
| | | | | al., Blood Cancer J. |
| | | | | 14(1): 24 (2024) |
| OS2966 | CD29 | | | Nigim et al., 2019, |
| | | | | Target. Oncol. 14: |
| | | | | 479-489. |
| osemitamab | CLDN18.2 | | | Qian et al., 2023, |
| | | | | Ann. Oncol. 34 |
| | | | | (Suppl. 2), S873, |
| | | | | 1560P. |
| otelixizumab | CD3 | Otelixizumab | MedChemExpress, | |
| | | | #HY-P99211 | |
| OTI14B1 | CD117 | KIT Mouse anti- | Fisher Scientific, | |
| | | Human, Clone: | #50-166-9129 | |
| | | OTI14B1, lyophilized, | | |
| | | TrueMAB ™ | | |
| OTI1B6 | | KIT Monoclonal | ThermoFisher, | |
| | | Antibody (OTI1B6), | #CF808721 | |
| | | TrueMAB ™ | | |
| OTI1E2 | | CD117 Mouse | OriGene, | |
| | | Monoclonal Antibody | #TA801037 | |
| | | [Clone ID: OTI1E2] | | |
| OTI1F6 | | KIT Monoclonal | ThermoFisher, | |
| | | Antibody (OTI1F6), | #CF801131 | |
| | | TrueMAB ™ | | |
| OTI2B12 | | CD117 Mouse | OriGene, | |
| | | Monoclonal Antibody | #CF808739 | |
| | | [Clone ID: OTI2B12] | | |
| OTI2B5 | | KIT Monoclonal | ThermoFisher, | |
| | | Antibody (OTI2B5), | #TA801047 | |
| | | TrueMAB ™ | | |
| OTI2C1D5 | | KIT Monoclonal | ThermoFisher, | |
| | | Antibody (OTI2C1D5), | #CF801043 | |
| | | TrueMAB ™ | | |
| OTI2C1H4 | | KIT Monoclonal | ThermoFisher, | |
| | | Antibody (OTI2C1H4), | #CF805183 | |
| | | TrueMAB ™ | | |
| OTI2E3 | | Monoclonal Mouse | LSBio, | |
| | | anti□Human c□Kit/ | #LS-C789071- | |
| | | CD117 Antibody | 100 | |
| | | (Carrier□free, | | |
| | | aa546□976, IHC, WB) | | |
| OTI3F9 | CD117 | KIT Monoclonal | ThermoFisher, | |
| | | Antibody (OTI3F9), | #TA801045 | |
| | | TrueMAB ™ | | |
| OTI4E4 | CD2 | available from multiple sources, including: | | |
| | | CD2 Monoclonal | ThermoFisher, | |
| | | Antibody (OTI4E4) | #TA500404 | |
| | | CD2 Mouse | Cambridge | |
| | | Monoclonal Antibody | Bioscience, | |
| | | [Clone ID: OTI4E4] | #TA500404 | |
| OTI4G8 | CD43 | ASPDH Monoclonal | ThermoFisher, | |
| | | Antibody (OTI4G8) | #MA5-27041 | |
| OTI6A4 | CD43 | PRRX1 Monoclonal | ThermoFisher, | |
| | | Antibody (OTI6A4) | #MA5-26579 | |
| OTI6F8 | CD117 | KIT Monoclonal | ThermoFisher, | |
| | | Antibody (OTI6F8), | #CF808746 | |
| | | TrueMAB ™ | | |
| OTI9A11 | CD117 | CD117 Mouse | OriGene, | |
| | | Monoclonal Antibody | #CF801205 | |
| | | [Clone ID: OTI9A11] | | |
| OX7 | CD90 | available from multiple sources, including: | | |
| | | CD90 Monoclonal | ThermoFisher, | |
| | | Antibody (OX-7) | #MA1-81491 | |
| | | Thy-1/CD90 Antibody | Santa Cruz | |
| | | (OX7) | Biotechnology, | |
| | | | #sc-53116 | |
| OX-45 | CD48 | CD48 Monoclonal | ThermoFisher, | |
| | | Antibody (OX-45), PE | #MA5-17528 | |
| OX-104 | CD200 | available from multiple sources, including: | | |
| | | CD200 Monoclonal | ThermoFisher, | |
| | | Antibody (OX104), | #14-9200-82 | |
| | | eBioscience ™ | | |
| | | CD200 antibody \| OX- | Bio-Rad, | |
| | | 104 | #MCA1960 | |
| pavurutamab | CD3 | Pavurutamab | MedChemExpress, | |
| | | | #HY-P99814 | |
| PD7/26 | CD45RB | CD45RB Monoclonal | ThermoFisher, | |
| | | Antibody (PD7/26), | #14-9458-82 | |
| | | eBioscience ™ | | |
| pembrolizumab | PD-1 | available from multiple sources, including: | | |
| | | Pembrolizumab | MedChemExpress, | |
| | | | #HY-P9902 | |
| | | | Evidentic, | |
| | | | #6302603D04 | |
| penpulimab | PD-1 | available from multiple sources, including: | | |
| | | Penpulimab | AbMole, | |
| | | | #M25087 | |
| | | Penpulimab (Anti- | #A3034 | |
| | | PDCD1/PD-1/CD279 | Selleckchem, | |
| peresolimab | PD-1 | Peresolimab | MedChemExpress, | |
| | | | #HY-P9993 | |
| PF-03475952 | CD44 | | | Runnels et al., 2010, |
| | | | | Adv. Ther. 27(3): |
| | | | | 168-180. |
| PG-M1 | CD68 | available from multiple sources, including: | | |
| | | Anti-CD68 antibody | Abcam, #ab783 | |
| | | [PG-M1] | | |
| | | CD68 Monoclonal | ThermoFisher, | |
| | | Antibody (PG-M1) | #MA5-12407 | |
| PI37012 | TREM2 | | | WO 2019/118513 |
| pidilizumab | PD-1 | Anti-Human PD- | Creative Biolabs, | |
| | | 1/PDCD1/CD279 | #TAB-900 | |
| | | Recombinant Antibody | | |
| | | (Pidilizumab) | | |
| pimivalimab | PD-1 | Pimivalimab | MedChemExpress, | |
| | | | #HY-P99887 | |
| pinatuzumab | CD22 | Pinatuzumab | MedChemExpress, | |
| | | | #HY-P99230 | |
| praluzatamab | CD166 | | | Boni et al., 2022, |
| | | | | Clin. Cancer Res. |
| | | | | 28(10): 2020-2029. |
| prezalumab | CD28 | Prezalumab | MedChemExpress, | |
| | | | #HY-P99414 | |
| priliximab | CD4 | available from multiple sources, including: | | |
| | | Priliximab | MedChemExpress, | |
| | | | #HY-P99790 | |
| | | Anti-Human CD4 | Creative Biolabs, | |
| | | Recombinant Antibody | #TAB-164 | |
| | | (Priliximab) | | |
| PRO1160 | CD70 | | | Jonasch et al., |
| | | | | 2023, Oncologist |
| | | | | 28(Suppl 1): S14- |
| | | | | S15. |
| prolgolimab | PD-1 | Prolgolimab | MedChemExpress, | |
| | | | #HY-P99807 | |
| promiximab | CD56 | Human Anti-NCAM1 | Creative Biolabs, | |
| | | Recombinant Antibody | #HPAB-N0187- | |
| | | | YC | |
| PRS-343 | CD137 | | | Hinner et al., 2016, |
| | | | | Cancer Immunol. |
| | | | | Res. 4 |
| | | | | (11_Supplement): |
| | | | | B016. |
| pucotenlimab | PD-1 | Pucotenlimab | MedChemExpress, | |
| | | | #HY-P99938 | |
| Q-1802 | CLDN18.2 | | | Yk et al., 2023, J. |
| | | | | Clin. Oncol. 41 (4 |
| | | | | suppl.), 382. |
| QBEND/10 | CD34 | CD34 Monoclonal | ThermoFisher, | |
| | | Antibody (QBEND/10), | #MA1-10205 | |
| | | PE | | |
| QL1706 | PD-1 | | | Zhang et al., 2021, |
| | CTLA-4 | | | Cancer Res. |
| | | | | 81(13_Suppl): |
| | | | | CT119. |
| quavonlimab | CTLA-4 | Quavonlimab | MedChemExpress, | |
| | | | #HY-P99809 | |
| r18D11 | CD14 | Anti-Human CD14 | Creative Biolabs, | |
| | | Recombinant Antibody | #TAB-1583CL | |
| | | (18D11) | | |
| ragifilimab | GITR | Ragifilimab | MedChemExpress, | |
| | | | #HY-P99812 | |
| RAVB3 | CD8 | available from multiple sources, including: | | |
| | | CD8 alpha Monoclonal | ThermoFisher, | |
| | | Antibody (RAVB3) | #MA5-17048 | |
| | | CD8 (RAVB3) | Santa Cruz | |
| | | | Biotechnology, | |
| | | | #sc-32812 | |
| RC118 | CLDN18.2 | | | Liu et al., 2024, Ann. |
| | | | | Oncol. 35 (Suppl. 2), |
| | | | | S903, 1456P. |
| REA101 | SSEA-4 | SSEA-4 Antibody, anti- | Miltenyi Biotec, | |
| | | human, PE, | #130-122-914 | |
| | | REAfinity ™ | | |
| REA157 | PODXL | TRA-1-60 Antibody, | Miltenyi Biotec, | |
| | | anti-human, PE, | #130-122-921 | |
| | | REAfinity ™ | | |
| REA246 | PODXL | TRA-1-81 Antibody, | Miltenyi Biotec, | |
| | | anti-human, PE, | #130-123-293 | |
| | | REAfinity ™ | | |
| REA233 | IL-21Rα | CD360 (IL-21R) | Miltenyi Biotec, | |
| | | Antibody, anti-human, | #130-126-094 | |
| | | PE, REAfinity ™ | | |
| REA333 | IL-12R | IL-12R β2 Antibody, | Miltenyi Biotec, | |
| | | anti-human, PE, | #130-120-068 | |
| | | REAfinity ™ | | |
| REA442 | CD166 | CD166 Antibody, anti- | Miltenyi Biotec, | |
| | | human, PE, | #130-118-349 | |
| | | REAfinity ™ | | |
| REA795 | SUSD2 | SUSD2 Antibody, anti- | Miltenyi Biotec, | |
| | | human, PE, | #130-111-641 | |
| | | REAfinity ™ | | |
| REA844 | CD271 | CD271 (LNGFR) | Miltenyi Biotec, | |
| | | Antibody, anti-human, | #130-112-601 | |
| | | PE, REAfinity ™ | | |
| REA877 | CD10 | CD10 Antibody, anti- | Miltenyi Biotec, | |
| | | human, pure, | #130-124-312 | |
| | | REAfinity ™ | | |
| REA897 | CD90 | CD90 Antibody, anti- | Miltenyi Biotec, | |
| | | human, PE, | #130-114-860 | |
| | | REAfinity ™ | | |
| REA1060 | CD29 | CD29 Antibody, anti- | Miltenyi Biotec, | |
| | | human, PE, | #130-118-121 | |
| | | REAfinity ™ | | |
| REA1067 | CD200 | CD200 Antibody, anti- | Miltenyi Biotec, | |
| | | human, PE, | #130-118-129 | |
| | | REAfinity ™ | | |
| REA1164 | CD34 | CD34 Antibody, anti- | Miltenyi Biotec, | |
| | | human, PE, | #130-120-515 | |
| | | REAfinity ™ | | |
| REAL219 | MSCA-1 | MSCA-1 Antibody, anti- | Miltenyi Biotec, | |
| | | human, PE, | #130-119-917 | |
| | | REAlease ® | | |
| REAL709 | CD271 | CD271 (LNGFR) | Miltenyi Biotec, | |
| | | Antibody, anti-human, | #130-125-053 | |
| | | PE, REAlease ® | | |
| REGN5459 | BCMA | | | U.S. Pat No. 11,384, 153 |
| REGN4659 | CTLA-4 | | | Burova et al., 2018, |
| | | | | Cancer Res. 78 |
| | | | | (13_Supplement): |
| | | | | 3824. |
| REGN6569 | GITR | | | Lakhani et al., 2022, |
| | | | | IOTECH 16 (S1): |
| | | | | 196TiP. |
| REGN7257 | CD132 | | | Le Floch-Ramondou |
| | | | | et al., 2022, Blood |
| | | | | 140 (Supplement 1): |
| | | | | 473-474. |
| relatlimab | LAG-3 | Relatlimab | MedChemExpress, | |
| | | | #HY-P99156 | |
| retifanlimab | PD-1 | Retifanlimab | MedChemExpress, | |
| | | | #HY-P99941 | |
| RG7356 | CD44 | Anti-Human CD44 | Creative Blolabs, | |
| | | Recombinant Antibody | #TAB-128CL | |
| | | (RG7356) | | |
| rituximab | CD20 | available from multiple sources, including: | | |
| | | BD Pharmingen ™ | BD Biosciences, | |
| | | Purified NA/LE Anti- | #569496 | |
| | | Human CD20 | | |
| | | Rituximab Biosimilar | | |
| | | HUMAN ANTI CD20 | BioRad, | |
| | | (RITUXIMAB | #MCA6091 | |
| | | BIOSIMILAR) | | |
| | | Rituximab | MedChemExpress, | |
| | | | #HY-P9913 | |
| RIV-11 | CD8 | available from multiple sources, including: | | |
| | | Anit-CD8 Antibody | Antibodies.com, | |
| | | (RIV-11) | #A115691 | |
| | | CD8 Recombinant | Aviva Systems | |
| | | Antibody( RIV-11) | Biology, | |
| | | | #OAAV00971 | |
| RM2051 | CD45 | Anti-CD45 antibody | Abcam, | |
| | | [RM2051] | #ab318154 | |
| RO5429083 | CD44 | | | Perez et al., 2012, |
| | | | | Cancer Res. 72 |
| | | | | (8_Suppl.): 2521. |
| RO7121661 | PD-1 | | | Clinical Trial ID: |
| | TIM-3 | | | NCT04785820 |
| RO7247669 | PD-1 | | | Clinical Trial ID: |
| | LAG-3 | | | NCT04785820 |
| RO7296682 | CD25 | Research Grade Anti- | AntibodySystem, | |
| | | Human CD25/IL2RA | #DHB95808 | |
| | | (RO7296682) | | |
| rocatinlimab | OX40 | Rocatinlimab | MedChemExpress, | |
| | | | #HY-P99955 | |
| RPA-2.10 | CD2 | available from multiple sources, including: | | |
| | | Purified anti-human | BioLegend, | |
| | | CD2 antibody | #300202 | |
| | | CD2 Monoclonal | ThermoFisher, | |
| | | Antibody (RPA-2.10) | #14-0029-82 | |
| | | Anti-Human CD2 | Stemcell | |
| | | Antibody, Clone RPA- | Technologies, | |
| | | 2.10 | #60007 | |
| RPA-T8 | CD8α | available from multiple sources, including: | | |
| (CT8) | | MOUSE ANTI HUMAN | Bio-Rad, | |
| | | CD8 | #MCA4609T | |
| | | CD8a Monoclonal | ThermoFisher, | |
| | | Antibody (RPA-T8), | #17-0088-42 | |
| | | APC | | |
| RTX-003 | CD25 | | | “Nature-inspired, |
| | | | | antibody discovery |
| | | | | driven by AI.” |
| | | | | Nature, December 2020, |
| | | | | B44. |
| rulonilimab | PD-1 | Rulonilimab | MedChemExpress, | |
| | | | #HY-P99895 | |
| RW03 | CD133 | | | Vora et al., 2020, |
| | | | | Cell Stem Cell |
| | | | | 26(6): 832-844.e6 |
| sabatolimab | TIM-3 | Sabatolimab | MedChemExpress, | |
| | | | #HY-P99044 | |
| samalizumab | CD200 | Samalizumab | MedChemExpress, | |
| | | | #HY-P99400 | |
| SAR445514 | BCMA | | | US 2024/0034816 |
| sasanlimab | PD-1 | Sasanlimab (Anti- | Selleckchem, | |
| | | PDCD1/PD-1/ | #A3038 | |
| | | CD279) | | |
| sdA1 | CD16a | | | WO 2018/039626 |
| sdA2 | CD16a | | | WO 2018/039626 |
| SEA-BCMA | BCMA | | | U.S. Pat No. 11,078,291 |
| selicrelumab | CD40 | Selicrelumab | MedChemExpress, | |
| | | | #HY-P99046 | |
| semzuvolimab | CD4 | Semzuvolimab | MedChemExpress, | |
| | | | #HY-P9998 | |
| serplulimab | PD-1 | Serplulimab (Anti- | Selleckchem, | |
| | | PDCD1/PD-1/ | #A2381 | |
| | | CD279) | | |
| SG001 | PD-1 | | | Clinical Trial ID: |
| | | | | NCT04886700 |
| SGN-CD70A | CD70 | | | Sandall et al., 2014, |
| | | | | Cancer Res. 74 |
| | | | | (19_Supplement): |
| | | | | 2647. |
| SHR-1702 | TIM-3 | | | Clinical Trial ID: |
| | | | | NCT03871855 |
| SHR-1802 | LAG-3 | | | Deng et al., 2023, |
| | | | | Ther. Adv. Med. |
| | | | | Oncol. 15: |
| | | | | 17588359231186025. |
| SIDI8BEE | CD8β | available from multiple sources, including: | | |
| | | CD8b Monoclonal | ThermoFisher, # | |
| | | Antibody (SIDI8BEE) | 14-5273-82 | |
| | | Mouse Anti-CD8B | CreativeBiolabs, | |
| | | Recombinant Antibody | #CBMAB- | |
| | | (SIDI8BEE) | C10732-LY | |
| sindelizumab | PD-1 | | | Li et al., 2022, Front. |
| | | | | Pharmacol. 13: |
| | | | | 918709. |
| sintilimab | PD-1 | Sintilimab | MedChemExpress, | |
| | | | #HY-P99048 | |
| siplizumab | CD2 | available from multiple sources, including: | | |
| | | Siplizumab | Medchem | |
| | | | Express, #HY- | |
| | | | P99904 | |
| | | Anti-Human CD2 | Creative Biolabs, | |
| | | Recombinant Antibody | #TAB-104 | |
| SJ25C1 | CD19 | | | Bejcek et al., 1995, |
| | | | | Cancer Res. |
| | | | | 55: 2346-2351 |
| SK1 | CD8α | available from multiple sources, including: | | |
| | | Purified Anti-human | AAT Bioquest, | |
| | | CD8 Antibody *SK1* | #10081000 | |
| | | Anti-CD8 Mouse | Avantor (VWR), | |
| | | Monoclonal Antibody | #344702-BL | |
| | | [clone: SK1] | | |
| SKB315 | CLDN18.2 | | | Clinical Trial ID: |
| | | | | NCT05367635 |
| SLAM.4 | CD150 | CD150 Monoclonal | ThermoFisher, | |
| | | Antibody (SLAM.4) | #MA5-46198 | |
| SM03 | CD22 | | | Wong et al., 2022, J. |
| | | | | Immunol. 208(12): |
| | | | | 2726-2737. |
| SOT102 | CLDN18.2 | | | Spisek, 2023, |
| | | | | ESMO Open 8(1), |
| | | | | Suppl. 2, 101196, |
| | | | | 2P. |
| sotigalimab | CD40 | Sotigalimab | MedChemExpress, | |
| | | | #HY-P99049 | |
| SP-16 | CD8 | available from multiple sources, including: | | |
| | | Anti-CD8 monoclonal | ThermoFisher, | |
| | | antibody (SP-16) | #MA5-14548 | |
| | | | Novus | |
| | | | Biologicals, | |
| | | | #NBP2-26484 | |
| SP34-2 | CD3 | BD Pharmingen ™ | BD Biosciences, | |
| | | APC-Cy ™7 Mouse | #557757 | |
| | | Anti-Human CD3 | | |
| SP55 | CD43 | CD43 Monoclonal | ThermoFisher, | |
| | | Antibody (SP55) | #MA5-16339 | |
| spartalizumab | PD-1 | Spartalizumab | MedChemExpress, | |
| | | | #HY-P9972 | |
| SPM504 | CD45RA | Anti-CD45RA antibody | Abcam, | |
| | | [SPM504] | #ab231426 | |
| SPX-101 | CLDN18.2 | | | Zhu et al., 2020, |
| | | | | Cancer Res. 80 |
| | | | | (16_Supplement): |
| | | | | 3361. |
| SR1 | CD117 | Anti-Human KIT | Creative Biolabs, | |
| | | Recombinant Antibody | #TAB-467LC | |
| | | (SR1) | | |
| ST04-99 | CD117 | CD117/c-kit Rabbit | Fisher Scientific, | |
| | | anti-Human, Clone: | #NBP267531 | |
| | | ST04-99, Novus | | |
| | | Biologicals ™ | | |
| STRO-1 | Stro-1 | STRO-1 Monoclonal | ThermoFisher, | |
| | | Antibody (STRO-1) | #39-8401 | |
| STRO-4 | Hsp90 | | | Gronthos et al., |
| | | | | 2009, Stem Cells |
| | | | | Dev. 18(9): 1253-62. |
| surzebiclimab | TIM-3 | Surzebiclimab | MedChemExpress, | |
| | | | #HY-P99962 | |
| Sym022 | LAG-3 | Research Grade Anti- | AntibodySystem, | |
| | | Human CD223/LAG3 | #DHD30423 | |
| | | (Sym022) | | |
| Sym023 | TIM-3 | | | Lindsted et al., |
| | | | | 2018, Cancer Res. 78 |
| | | | | (13_Supplement): |
| | | | | 5629. |
| Sym024 | CD73 | | | Jakobsen et al., |
| | | | | 2021, Cancer Res. 81 |
| | | | | (13_Supplement): |
| | | | | 1797. |
| SYSA180 | CLDN18.2 | | | Wang et al., 2023, J. |
| | | | | Clin. Oncol. 41 (16 |
| | | | | suppl.), 3016. |
| T200/797 | CD45RO | Anti-CD45RO antibody | Abcam, | |
| | | [T200/797] | #ab216024 | |
| T3-3A1 | CD7 | CD7 (T-Cell Leukemia | ThermoFisher, | |
| | | Marker) Monoclonal | #924-MSM5-PO | |
| | | Antibody (T3-3A1) | | |
| T6.3 | CD2 | available from multiple sources, including: | | |
| | | Mouse Anti-CD2 | Creative Biolabs, | |
| | | Recombinant Antibody | #ZG-0903F | |
| | | (clone T6.3) | | |
| | | Monoclonal Mouse | LS Bio, | |
| | | anti□Human CD2 | #LS□C140259 | |
| | | Antibody (clone T6.3) | | |
| T595 | CD117 | CD117 Mouse | OriGene, | |
| | | Monoclonal Antibody | #TA355261 | |
| | | [Clone ID: T595] | | |
| TAB-071CL | BMPR2 | Anti-Human BMPR | Creative Biolabs, | |
| | | Recombinant Antibody | #TAB-071CL | |
| talquetamab | GPRC5D | | | Pillarisetti et al., |
| | | | | Blood 135: 1232-43 |
| | | | | (2020) |
| tavolimab | OX40 | Tavolimab | ThermoFisher, | |
| | | Recombinant Human | #MA5-42300 | |
| | | Monoclonal Antibody | | |
| TB19 | CD73 | InVivoMAb Anti-Human | AntibodySystem, | |
| | | CD73/NT5E Antibody | #VHD46601 | |
| | | (TB19) | | |
| TB38 | CD73 | InVivoMAb Anti-Human | AntibodySystem, | |
| | | CD73/NT5E Antibody | #VHD46602 | |
| | | (TB38) | | |
| tebotelimab | LAG-3 | Tebotelimab | MedChemExpress, | |
| | PD-1 | | #HY-P99573 | |
| teclistamab | CD3 | Teclistamab | MedChemExpress, | |
| | | | #HY-P99392 | |
| telazorlimab | OX40 | Telazorlimab | MedChemExpress, | |
| | | | #HY-P99570 | |
| teplizumab | CD3 | Teplizumab | MedChemExpress, | |
| | | | #HY-P99222 | |
| TG6050 | CTLA-4 | | | Marchand et al., |
| | | | | 2023, Cancer Res. |
| | | | | 83 (7_Supplement): |
| | | | | 694. |
| TH-69 | CD7 | Anti-CD7 [TH69] | Absolute | |
| | | | Antibody, | |
| | | | #Ab03054 | |
| theralizumab | CD28 | Theralizumab | MedChemExpress, | |
| | | | #HY-P9975 | |
| tifcemalimab | BTLA | | | Song et al., 2023, |
| | | | | Blood 142 |
| | | | | (Supplement 1): |
| | | | | 4458. |
| tiragolumab | TIGIT | | | Kim et al., 2023, |
| | | | | JAMA Oncol. |
| | | | | 9(11): 1574-1582. |
| tisagenlecleucel | CD19 | | | Mueller et al., 2018, |
| | | | | Clin. Cancer Res. |
| | | | | 24 (24): 6175-6184. |
| tislelizumab | PD-1 | Tislelizumab | MedChemExpress, | |
| | | | #HY-P99052 | |
| TNB-383B | BCMA | | | U.S. Pat No. 11,505,606 |
| tocilizumab | IL-6R | available from multiple sources, including: | | |
| | | IL6R antibody \| rhPM-1 | BioRad, | |
| | | | #MCA6106 | |
| | | Tocilizumab | MedChemExpress, | |
| | | | #HY-P9917 | |
| | | Tocilizumab | ThermoFisher, | |
| | | Recombinant Human | #MA5-48061 | |
| | | Monoclonal Antibody | | |
| | | (rhPM-1 (Tocilizumab)) | | |
| toripalimab | PD-1 | Toripalimab | MedChemExpress, | |
| | | | #HY-P9978 | |
| TORL-2-307-ADC | CLDN18.2 | | | Clinical Trial ID: |
| | | | | NCT05156866 |
| tositumomab | CD20 | available from multiple sources, including: | | |
| | | Human CD20 | R&D Systems, | |
| | | (Research Grade | #FAB10440R | |
| | | Tositumomab | | |
| | | Biosimilar) Alexa | | |
| | | Fluor ® 647-conjugated | | |
| | | Antibody | | |
| | | Tositumomab | ThermoFisher, | |
| | | Recombinant Mouse | #MA5-41763 | |
| | | Monoclonal Antibody | | |
| TQB2618 | TIM-3 | Research Grade Anti- | AntibodySystem, | |
| | | Human CD366/HAVCR2/TIM-3 | #DHJ28406 | |
| | | (TQB2618) | | |
| TQB2934 | BCMA | | | US 2023/0193292 |
| TRC205 | CD105 | | | U.S. Pat No. 9,926,375 |
| tregalizumab | CD4 | Tregalizumab | MedChemExpress, | |
| | | | #HY-P99327 | |
| tremelimumab | CTLA-4 | Tremelimumab | MedChemExpress, | |
| | | | #HY-P9918 | |
| TRX1 | CD4 | Anti-Human CD4 | Creative Biolabs, | |
| | | Recombinant Antibody | #TAB-168LC | |
| | | (TRX1) | | |
| TRX2 | CD8 | Oxford Therapeutic Antibody Centre, | | |
| | | Oxford University, Oxford, United | | |
| | | Kingdom | | |
| TRX518 | GITR | Research Grade Anti- | AntibodySystem, | |
| | | Human | #DHJ89807 | |
| | | CD357/TNFRSF18/GITR | | |
| | | (TRX518) | | |
| TS1/8 | CD2 | available from multiple sources, including: | | |
| | | CD2 Monoclonal | ThermoFisher, | |
| | | Antibody (TS1/8) | #MA5-44080 | |
| | | Purified anti-human | BioLegend, | |
| | | CD2 Antibody | #309202 | |
| TS2/18 | CD2 | available from multiple sources, including: | | |
| | | CD2 Monoclonal | ThermoFisher, | |
| | | Antibody (TS2/18) | #MA0200 | |
| | | CD2 Antibody | Santa Cruz Bio, | |
| | | (TS2/18.1.1) | #sc-19640 | |
| TTI-CD200 | CD200 | | | Rastogi et al., 2021, |
| | | | | Br. J. Haematol. |
| | | | | 193, 155-159. |
| TUSP-2 | Stro-1 | Anti-STRO-1 | Creative | |
| | | monoclonal antibody | Diagnostics, | |
| | | [Alexa Fluor ® 647] | #DMAB18656 | |
| UB-421 | CD4 | Research Grade | AntibodySystem, | |
| | | Semzuvolimab | #DHB95908 | |
| ublituximab | CD20 | available from multiple sources, including: | | |
| | | Ublituximab | MedChemExpress, | |
| | | | #HY-P99538 | |
| | | Ublituximab (Anti- | SelleckChem, | |
| | | CD20) | #A2807 | |
| | | Ublituximab | ThermoFisher, | |
| | | Recombinant Human | #MA5-41938 | |
| | | Monoclonal Antibody | | |
| UCART20 | CD20 | | | Kang et al., 2024, J. |
| | | | | Hem. Oncol. 17: 29. |
| UCHL1 | CD45RO | Anti-CD45RO antibody | Abcam, #ab23 | |
| | | [UCH-L1] | | |
| UCHT2 | CD5 | CD5 Monoclonal | ThermoFisher, | |
| | | Antibody (UCHT2), | #14-0059-82 | |
| | | eBioscience ™ | | |
| UCHT4 | CD8 | available from multiple sources, including: | | |
| | | CD8 Monoclonal | ThermoFisher, # | |
| | | Antibody (UCHT4) | 65204-1-IG | |
| | | Anti-CD8 [UCHT4] | Absolute | |
| | | | Antibody, | |
| | | | #Ab00694-2.0 | |
| uliledlimab | CD73 | Uliledlimab | MedChemExpress, | |
| | | | #HY-P99169 | |
| UMAB216 | CD117 | KIT Monoclonal | ThermoFisher, | |
| | | Antibody (UMAB216), | #UM800108CF | |
| | | UltraMAB ™ | | |
| UMCD2 | CD2 | available from multiple sources, including: | | |
| | | anti-human CD2 | Biohippo, | |
| | | | #BHA14700014 | |
| | | CD2 Monoclonal | Biotium, #1235 | |
| | | Mouse Antibody | | |
| | | (UMCD2) | | |
| urelumab (BMS- | CD137 | Urelumab | MedChemExpress, | |
| 663513) | | | #HY-P99055 | |
| ustekinumab | IL-12 | available from multiple sources, including: | | |
| | IL-23 | Ustekinumab Biosimilar - | Proteogenix, | |
| | | Anti-Human IL-12 IL- | #PX-TA1011 | |
| | | 23 mAb - Research | | |
| | | Grade | | |
| | | Ustekinumab (anti-IL- | SelleckChem, | |
| | | 12/IL-23) | #A2024 | |
| utomilumab | CD137 | Utomilumab | MedChemExpress, | |
| (PF-05082566) | | | #HY-P99056 | |
| varlilumab | CD27 | Varlilumab | ThermoFisher, | |
| | | Recombinant Human | #MA5-42030 | |
| | | Monoclonal Antibody | | |
| VB6-008 | CD44 | | | U.S. Pat No. 8,383,117 |
| veltuzumab | CD20 | available from multiple sources, including: | | |
| | | Veltuzumab | MedChemExpress, | |
| | | | #HY-P99224 | |
| | | Veltuzumab Biosimilar - | Proteogenix, | |
| | | Anti- CD20 receptors | #PX-TA1616 | |
| | | mAb - Research | | |
| | | Grade | | |
| | | Veltuzumab | ThermoFisher, | |
| | | Recombinant Human | #MA5-42301 | |
| | | Monoclonal Antibody | | |
| VIB9600 | CD32A | | | Chen et al., 2019, |
| | | | | Ann. Rheum. Dis. |
| | | | | 78(2): 228-237. |
| vibecotamab | CD3 | Vibecotamab | Fisher Scientific, | |
| | | Recombinant Human | #PIMA542248 | |
| | | Monoclonal Antibody, | | |
| | | Invitrogen ™ | | |
| vibostolimab | TIGIT | | | Niu et al., 2022, |
| (MK-7684) | | | | Ann. Oncol. 33(2): |
| | | | | 169-180. |
| visilizumab | CD3 | Anti-CD3E | Creative Biolabs, | |
| | | Recombinant Antibody | #TAB-714 | |
| | | (Visilizumab) | | |
| vonlerizumab | OX40 | Vonlerizumab (Anti- | SelleckChem, | |
| | | TNFRSF4/OX40/ | #A2325 | |
| | | CD134) | | |
| vorsetuzumab | CD27 | Vorsetuzumab | MedChemExpress, | |
| | CD70 | | #HY-P99273 | |
| vudalimab | PD-1 | Research Grade | AntibodySystem, | |
| | | Vudalimab | #DHH02222 | |
| vunakizumab | CD27 | Anti-Human IL17A | Creative Biolabs, | |
| | | Recombinant Antibody | #TAB-458CQ | |
| | | (Vunakizumab) | | |
| W3/13HLK | CD43 | CD43 Monoclonal | ThermoFisher, | |
| | | Antibody (W3/13HLK), | #MA5-17386 | |
| | | PE | | |
| W5C5 | SUSD2 | Purified anti-human | BioLegend, | |
| | | SUSD2 Antibody | #327401 | |
| W8B2 | MSCA-1 | MSCA-1 Antibody, anti- | Miltenyi Biotec, | |
| | | human | #130-093-595 | |
| WT1 | CD7 | Mouse Anti-CD7 | Creative Biolabs, | |
| | | Recombinant Antibody | #CBMAB- | |
| | | (WT1) | C10581-LY | |
| WV078 | BCMA | | | U.S. Pat No. 11,492,409 |
| X9C3 | MSCA-1 | Mouse anti-Human | Creative | |
| | | MSCA-1 monoclonal | Diagnostics, | |
| | | antibody, PE | #CABT-B12374 | |
| XTX101 | CTLA-4 | | | Jenkins et al., 2023, |
| | | | | J. Immunother. |
| | | | | Cancer 11(12): |
| | | | | e007785. |
| Y1/82A | CD68 | CD68 antibody \| | Bio-Rad, | |
| | | Y1/82A | #MCA6014GA | |
| YB5.B8 | CD117 | CD117 (c-Kit) | ThermoFisher, | |
| | | Monoclonal Antibody | #14-1179-82 | |
| | | (YB5.B8), | | |
| | | eBioscience ™ | | |
| YH001 | CTLA-4 | | | Ganju et al., 2021, |
| | | | | J. Clin. Oncol. 39 |
| | | | | (15_suppl): 2577. |
| YTC182.20 | CD8 | Anti-CD8 alpha | Abcam, | |
| | | antibody [YTC182.20] | #ab60076 | |
| YTH3.2.6 | CD7 | Anti-CD7 [YTH3.2.6] | Absolute | |
| | | | Antibody, | |
| | | | #Ab01019 | |
| zalifrelimab | CTLA-4 | Zalifrelimab | MedChemExpress, | |
| | | | #HY-P99514 | |
| zanolimumab | CD4 | Zanolimumab | ThermoFisher, | |
| | | Recombinant Human | #MA5-41819 | |
| | | Monoclonal Antibody | | |
| zimberelimab | PD-1 | Zimberelimab | MedChemExpress, | |
| | | | #HY-P99109 | |
| zolbetuximab | CLDN18.2 | | | Sahin et al., 2018, |
| (IMAB362) | | | | Eur. J. Cancer 100, |
| | | | | 17-26. |

We claim:

1. A nucleic acid molecule comprising in 5′ to 3′ order,
i. a 5′ untranslated region (UTR), an open reading frame (ORF), and a 3′ UTR wherein the 3′ UTR comprises RPS3A-PNLIP tandem 3′ UTR (SEQ ID NO: 43) or PNLIP-RPS3A tandem 3′ UTR (SEQ ID NO: 44) or a variant thereof having ≥95% sequence identity to SEQ ID NO: 43 or SEQ ID NO: 44; or
ii. a 5′ untranslated region (UTR), an open reading frame (ORF), and a 3′ UTR wherein the 5′ UTR and 3′ UTR comprise UTR sequences, or a variant thereof having ≥95% sequence identity to the UTR sequences, selected from one of the following pairs:
a) mouse β-globin (mHBB) 5′ UTR (SEQ ID NO: 21) and pancreatic triacylglycerol lipase (PNLIP) 3′ UTR (SEQ ID NO: 41), ribosomal protein S3A (RPS3A) 3′ UTR (SEQ ID NO: 42), RPS3A-PNLIP tandem 3′ UTR (SEQ ID NO: 43), PNLIP-RPS3A tandem 3′ UTR (SEQ ID NO: 44), human hemoglobin subunit α1 (hHBA1) 3′ UTR (SEQ ID NO: 45) or human hemoglobin subunit α1 with 3 miRNA122 binding sites (hHBA1-3× miR122 bs) 3′ UTR (SEQ ID NO: 46);
b) carboxypeptidase A1 (CPA1) 5′ UTR (SEQ ID NO: 22) and CPA1 3′ UTR (SEQ ID NO: 47) wherein the ORF does not encode CPA1 or a protein having ≥60% sequence identity with CPA1;
c) defensin alpha 3 (DEFA3) 5′ UTR (SEQ ID NO: 23) and DEFA3 3′ UTR (SEQ ID NO: 48) wherein the ORF does not encode DEFA3 or a protein having ≥60% sequence identity with DEFA3;
d) human albumin (hAlb) 5′ UTR (SEQ ID NO: 24) and hHBA1 3′ UTR (SEQ ID NO: 45);
e) eIF4G aptamer ×1 (SEQ ID NO: 26) and hHBA1 3′ UTR (SEQ ID NO: 45); or
f) aptamer control (SEQ ID NO: 27) and hHBA1 3′ UTR (SEQ ID NO: 45); or
iii. a 5′ untranslated region (UTR), an open reading frame (ORF), and a 3′ UTR wherein the 5′ UTR and 3′ UTR comprise UTR sequences, or a variant thereof having ≥95% sequence identity to the UTR sequences, selected from one of the following pairs:
a) mouse β-globin (mHBB) 5′ UTR (SEQ ID NO: 11) and pancreatic triacylglycerol lipase (PNLIP) 3′ UTR (SEQ ID NO: 31), ribosomal protein S3A (RPS3A) 3′ UTR (SEQ ID NO: 32), RPS3A-PNLIP tandem 3′ UTR (SEQ ID NO: 33), PNLIP-RPS3A tandem 3′ UTR (SEQ ID NO: 34), human hemoglobin subunit α1 (hHBA1) 3′ UTR (SEQ ID NO: 35) or human hemoglobin subunit α1 with 3 miRNA122 binding sites (hHBA1-3× miR122 bs) 3′ UTR (SEQ ID NO: 36);
b) carboxypeptidase A1 (CPA1) 5′ UTR (SEQ ID NO: 12) and CPA1 3′ UTR (SEQ ID NO: 37);
c) defensin alpha 3 (DEFA3) 5′ UTR (SEQ ID NO: 13) and DEFA3 3′ UTR (SEQ ID NO: 38);
d) human albumin (hAlb) 5′ UTR (SEQ ID NO: 14) and hHBA1 3′ UTR (SEQ ID NO: 35);
e) hemoglobin subunit alpha 1 (HBA) 5′ UTR (SEQ ID NO: 15) and amino-terminal enhancer of split and mitochondrially encoded 12S rRNA(AES-mtRNR1) 3′ UTR (SEQ ID NO: 39);
f) eIF4G aptamer ×1 (SEQ ID NO: 16) and hHBA1 3′ UTR (SEQ ID NO: 35);
g) aptamer control (SEQ ID NO: 27) and hHBA1 3′ UTR (SEQ ID NO: 45); or
h) a synthetic 5′ UTR (SEQ ID NO: 18) and hHBA1 3′ UTR (SEQ ID NO: 35).

2. A nucleic acid molecule comprising in 5′ to 3′ order, a 5′ untranslated region (UTR), an open reading frame (ORF) encoding a chimeric antigen receptor (CAR), and a 3′ UTR wherein the 5′ UTR and 3′ UTR comprise UTR sequences, or a variant thereof having ≥95% sequence identity to the UTR sequences, selected from one of the following pairs:
a) hemoglobin subunit alpha 1 (HBA) 5′ UTR (SEQ ID NO: 25) and amino-terminal enhancer of split and mitochondrially encoded 12S rRNA(AES-mtRNR1) 3′ UTR (SEQ ID NO: 49);
b) a synthetic 5′ UTR (SEQ ID NO: 28) and hHBA1 3′ UTR (SEQ ID NO: 45); or
c) mouse β-globin (mHBB) 5′ UTR (SEQ ID NO: 21) and human hemoglobin subunit α1 (hHBA1) 3′ UTR (SEQ ID NO: 45).

3. The nucleic acid molecule of claim 1, comprising an mRNA molecule.

4. The nucleic acid molecule of claim 1, comprising a DNA molecule encoding an mRNA molecule.

5. The mRNA of claim 3, wherein one or a plurality of uridine nucleosides is substituted with a modified nucleoside.

6. The nucleic acid molecule of claim 1, wherein the ORF encodes a chimeric antigen receptor (CAR).

7. The nucleic acid molecule of claim 6 encoding a CAR, wherein the CAR is an anti-CD19 CAR, an anti-CD20 CAR, an anti-BCMA CAR, or an anti-fibroblast activation protein (FAP) CAR.

8. The nucleic acid molecule of claim 7, wherein the anti-CD19 binding domain has the amino acid sequence of SEQ ID NO: 259.

9. The nucleic acid molecule of claim 8, wherein the anti-CD19 CAR has the amino acid sequence of SEQ ID NO: 4 or SEQ ID NO: 5.

10. The nucleic acid molecule of claim 9, wherein the ORF encoding the anti-CD19 CAR has a nucleotide sequence of SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, or a sequence having at least 95, 96, 97, 98, or 99% sequence identity thereto.

11. A nucleic acid molecule, wherein the nucleic acid molecule is an mRNA of RM_61321 (SEQ ID NO: 113), RM_61324 (SEQ ID NO: 114), RM_61326 (SEQ ID NO: 115), RM_61329 (SEQ ID NO: 116), RM_61330 (SEQ ID NO: 117), RM_61334 (SEQ ID NO: 118), RM_61340 (SEQ ID NO: 119), RM_61341 (SEQ ID NO: 120), RM_61347 (SEQ ID NO: 121), RM_61348 (SEQ ID NO: 122), RM_61349 (SEQ ID NO: 123), RM_61350 (SEQ ID NO: 124), RM_61351 (SEQ ID NO: 125), RM_61352 (SEQ ID NO: 126), RM_61353 (SEQ ID NO: 127), RM_61355 (SEQ ID NO: 128), RM_61356 (SEQ ID NO: 129), RM_61357 (SEQ ID NO: 130), RM_61358 (SEQ ID NO: 131), RM_61359 (SEQ ID NO: 132), RM_61360 (SEQ ID NO: 133), RM_61378 (SEQ ID NO: 136), RM_61379 (SEQ ID NO: 137), RM_61411 (SEQ ID NO: 138), RM_61449 (SEQ ID NO: 139), RM_61450 (SEQ ID NO: 140), RM_61451 (SEQ ID NO: 141), RM_61452 (SEQ ID NO: 142), RM_61453 (SEQ ID NO: 143), RM_61454 (SEQ ID NO: 144), RM_61455 (SEQ ID NO: 145), RM_61456 (SEQ ID NO: 146), RM_61457 (SEQ ID NO: 147), RM_61458 (SEQ ID NO: 148), RM_61459 (SEQ ID NO: 149), RM_61460 (SEQ ID NO: 150), RM_61461 (SEQ ID NO: 151), RM_61462 (SEQ ID NO: 152), RM_61463 (SEQ ID NO: 153), RM_61465 (SEQ ID NO: 155), RM_61466 (SEQ ID NO: 156), RM_61467 (SEQ ID NO: 157), RM_61468 (SEQ ID NO: 158), RM_61482 (SEQ ID NO: 159), RM_61483 (SEQ ID NO: 160), RM_61484 (SEQ ID NO: 161), RM_61485 (SEQ ID NO: 162), RM_61486 (SEQ ID NO: 163), RM_61487 (SEQ ID NO: 164), RM_61488 (SEQ ID NO: 165), RM_61489 (SEQ ID NO: 166), RM_61501 (SEQ ID NO: 167), RM_61514 (SEQ ID NO: 170), RM_61515 (SEQ ID NO: 171), RM_61519 (SEQ ID NO: 175), RM_61520 (SEQ ID NO: 176), RM_61639 (SEQ ID NO: 178), RM_61653 (SEQ ID NO: 316), RM_61654 (SEQ ID NO: 317), RM_61655 (SEQ ID NO: 318), RM_61656 (SEQ ID NO: 319), RM_61657 (SEQ ID NO: 321), RM_61658 (SEQ ID NO: 322), RM_61659 (SEQ ID NO: 323), RM_61660 (SEQ ID NO: 324), or a DNA molecule encoding said mRNA molecule.

12. A cell comprising the nucleic acid molecule of claim 1, wherein the nucleic acid molecule is an mRNA molecule, wherein the cell does not comprise DNA encoding the mRNA molecule.

13. A cell comprising a polypeptide encoded by the mRNA molecule of claim 4, wherein the cell does not comprise DNA encoding the mRNA molecule.

14. A pharmaceutical composition, comprising the nucleic acid molecule of claim 1, wherein the nucleic acid molecule is one or more mRNA molecules and the one or more mRNA molecules are encapsulated in one or more lipid nanoparticles (LNPs).

15. The pharmaceutical composition of claim 14, wherein the one or more LNPs has a lipid composition that comprises about 35 mol % to about 65 mol % an ionizable cationic lipid, about 7 mol % to about 13 mol % a phospholipid, about 0.5 mol % to about 3 mol % a PEG-lipid wherein the PEG-lipid comprises non-functionalized PEG-lipid or functionalized PEG-lipid and non-functionalized PEG-lipid, and about 27 mol % to about 50 mol % a sterol.

16. The pharmaceutical composition of claim 15, further comprising a binding moiety conjugated to the functionalized PEG-lipid of each of the one or more LNPs.

17. The pharmaceutical composition of claim 16, wherein each binding moiety comprises an antibody or antigen binding domain thereof.

18. The pharmaceutical composition of claim 17, wherein each binding moiety comprises an anti-CD8 antibody antigen binding domain that that has at least 90% identity with the amino acid sequence of the framework regions of SEQ ID NO: 188 or 206 wherein the VH comprises a heavy chain CDR1 (VH-CDR1) comprising the amino acid sequence RYTFTDYX₁LH (SEQ ID NO: 220) wherein X₁ is N, S, Q, or A, a VH-CDR2 comprising the amino acid sequence FIYPYX₁GGTG (SEQ ID NO: 221) or FIYPYX₂GGTG (SEQ ID NO: 222) wherein X₂ is N, Q, D, S, or A, and a VH-CDR3 having the amino acid sequence DHRYX₁EGVSFDY (SEQ ID NO: 223); and a light chain variable region (VL) comprising an amino acid sequence that has at least 90% identity with the amino acid sequence of the framework regions of SEQ ID NO: 194 or 212, wherein the VL comprises a CDR1 (VL-CDR1) comprising the amino acid sequence RASESVX₃GFGX₁SFMN wherein X₃ is D, E, S, or A (SEQ ID NO: 224), VL-CDR2 comprising the amino acid sequence LASX₂LES (SEQ ID NO: 225), and a VL-CDR3 having the amino acid sequence QQX₂X₂EX₃PYT (SEQ ID NO: 226).

19. The pharmaceutical composition of claim 18, wherein the antigen binding domain comprises a VL region having the amino acid sequence of SEQ ID NO: 196 and a VH region having the amino acid sequence of one of SEQ ID NOs: 190 or 202-204.

20. The pharmaceutical composition of claim 19, wherein the binding moiety is a whole antibody, wherein the whole antibody comprises a silenced Fc region having the amino acid sequence of SEQ ID NO: 218 or 219.

21. The pharmaceutical composition of claim 15, wherein a lipid composition of the LNP comprises:
a) about 40 mol % to about 62 mol % ionizable cationic lipid, about 7 mol % to about 13 mol % phospholipid, about 30 mol % to about 50 mol % sterol, about 0.5 mol % to about 3 mol % total functionalized and unfunctionalized PEG-lipid and about 0.1 mol % to 0.3 mol % functionalized PEG-lipid;
b) about 50 mol % CLCL, about 10 mol % phospholipid, about 38.5 mol % sterol, about 1.4 mol % non-functionalized PEG-lipid, and about 0.1 mol % functionalized PEG-lipid;
c) about 58 mol % CLCL, about 10 mol % phospholipid, about 30.5 mol % sterol, about 1.4 mol % non-functionalized PEG-lipid, and about 0.1 mol % functionalized PEG-lipid; or
d) about 62 mol % CLCL, about 10 mol % phospholipid, about 26.5 mol % sterol, about 1.4 mol % non-functionalized PEG-lipid, and about 0.1 mol % functionalized PEG-lipid.

22. The pharmaceutical composition of claim 21, wherein the lipid composition of the LNP is CICL1:distearoylphosphatidylcholine (DSPC):cholesterol (CHOL):unfunctionalized PEG-lipid:functionalized PEG-lipid [58:10:30.5:1.4:0.1], wherein the unfunctionalized PEG-lipid is 1,2-distearoyl-glycero-3-methoxypolyethylene glycol and the functionalized PEG-lipid is 1,2-distearoyl-glycero-3-phosphoethanolamine-N-[maleimide(polyethylene glycol)].

23. The pharmaceutical composition of claim 22, having the LNP comprising a binding moiety, wherein the binding moiety:
a) comprises an anti-CD8 antibody antigen binding domain that that has at least 90% identity with the amino acid sequence of the framework regions of SEQ ID NO: 188 or 206 wherein the VH comprises a heavy chain CDR1 (VH-CDR1) comprising the amino acid sequence RYTFTDYX₁LH (SEQ ID NO: 220) wherein X₁ is N, S, Q, or A, a VH-CDR2 comprising the amino acid sequence FIYPYX₁GGTG (SEQ ID NO: 221) or FIYPYX₂GGTG (SEQ ID NO: 222) wherein X₂ is N, Q, D, S, or A, and a VH-CDR3 having the amino acid sequence DHRYX₁EGVSFDY (SEQ ID NO: 223); and a light chain variable region (VL) comprising an amino acid sequence that has at least 90% identity with the amino acid sequence of the framework regions of SEQ ID NO: 194 or 212, wherein the VL comprises a CDR1 (VL-CDR1) comprising the amino acid sequence RASESVX₃GFGX₁SFMN wherein X₃ is D, E, S, or A (SEQ ID NO: 224), VL-CDR2 comprising the amino acid sequence LASX₂LES (SEQ ID NO: 225), and a VL-CDR3 having the amino acid sequence QQX₂X₂EX₃PYT (SEQ ID NO: 226);
b) is a whole antibody or antigen binding portion thereof that comprises a VL region having the amino acid sequence of SEQ ID NO: 196 and a VH region having the amino acid sequence of one of SEQ ID NOs: 190 or 202-204;
c) is a F(ab′) that comprises a VL region having the amino acid sequence of SEQ ID NO: 196 and a VH region having the amino acid sequence of one of SEQ ID NOs: 190 or 202-204;
d) is a F(ab′) that comprises a VL region having the amino acid sequence of SEQ ID NO: 196 and a VH region having the amino acid sequence of one of SEQ ID NOs: 190 or 202-204, and further comprises a S162C kappa chain substitution, a C214S kappa chain substitution, and either an IgG1 F174C substitution and an IgG1 CH1 C233S, or an IgG4 CH1 F174C substitution and an IgG4 CH1 C127S substitution, or
e) is a whole antibody wherein the heavy chain has the sequence of SEQ ID NO: 347 and the light chain has the sequence of SEQ ID NO: 348.

24. The pharmaceutical composition of claim 23, wherein the LNP encapsulates an mRNA comprising RM_61357 (SEQ ID NO: 130), RM_61461 (SEQ ID NO: 151), RM_61488 (SEQ ID NO: 165), and RM_61489 (SEQ ID NO: 166).

25. A method of treating a subject with cancer or a genetic disease or an autoimmune or fibrotic disease, comprising administering to the subject a therapeutically effective dose of the pharmaceutical composition of claim 14.

26. A method of depleting B cells in vivo comprising administering to the subject a therapeutically effective dose of the pharmaceutical composition of claim 14, wherein the one or more mRNA molecules encodes an anti-CD19 CAR, an anti-CD20 CAR, an anti-BCMA CAR, or a combination thereof.

27. A method of transiently transfecting a cell in vivo comprising administering a pharmaceutical composition of claim 14, to a mammalian subject.

28. The method of claim 27, wherein at least one of the one or more mRNAs encodes an anti-FAP CAR, an anti-CD19 CAR, an anti-CD20 CAR, an anti-BCMA CAR, or a combination thereof.

29. The nucleic acid molecule of claim 2, comprising an mRNA molecule.

30. The nucleic acid molecule of claim 2, comprising a DNA molecule encoding an mRNA molecule.
